(19) United States
(12) Patent Application Publication
(10) Pub. No.: US 2009/0233910 A1
(43) Pub. Date: Sep. 17, 2009

(54) Npy antagonists, preparation and uses

(75) Inventors: Iuliana Botez, Houilles (FR); Christelle David-Basei, Nanterre (FR); Nelly Gourlaouen, Chaville (FR); Eric Nicolai, Rueil Malmaison (FR); Fabrice Balavoine, Paris (FR); Gerard Valette, Lacroix-Falgarde (FR); Claudine Serradeil-Le Gal, Escalquens (FR)

Correspondence Address: BUCHANAN, INGERSOLL & ROONEY PC, POST OFFICE BOX 1404, ALEXANDRIA, VA, 22313-1404, US

(73) Assignee: CEREP, Paris (FR)

(21) Appl. No.: 11/918,470

(22) PCT Filed: Apr. 14, 2006
(86) PCT No.: PCT/FR2006/000829

§ 371 Date: Feb. 27, 2009

(30) Foreign Application Priority Data
Apr. 15, 2005 (FR) 0503795

(51) Int. Cl. A61K 31/551; C07D 243/08; A61P 25/02

(52) U.S. Cl. 514/218; 540/575

(57) ABSTRACT

The present invention concerns novel compounds, their preparation and their uses, therapeutic uses in particular. More specifically it concerns derivative compounds having at least two aromatic cycles, their preparation and their uses, in particular in the area of human or animal health. These compounds have an affinity for the biological receptors of neuropeptide Y, NPY, present in the central and peripheral nervous systems. The compounds of the invention are preferably NPY antagonists, and more particularly antagonists of sub-type NPY Y1, and can therefore be used for the therapeutic or prophylactic treatment of any disorder involving NPY. The present invention also concerns pharmaceutical compositions containing said compounds, their preparation and their uses, as well as treatment methods using said compounds.

The present invention relates to novel compounds, their preparation and their uses, in particular their therapeutic uses. It relates more particularly to compounds having at least two aromatic cycles, their preparation and their uses, especially in the area of human or animal health. These compounds have an affinity for receptors of neuropeptide Y, NPY, present in the central and peripheral nervous systems. The compounds of the invention are preferably NPY antagonists, and more especially antagonists of sub-type NPY Y1, and can therefore be used for the therapeutic or prophylactic treatment of disorders involving NPY overexpression. The present invention also concerns pharmaceutical compositions containing said compounds, their preparation and their uses, as well as treatment methods using said compounds.

Neuropeptide Y (NPY) consists of 36 amino acids and was first isolated in 1982 from porcine brain. This neuropeptide belongs to a family of peptides also including peptide YY (PYY) and the pancreatic peptide (PP). It acts on several types of G-protein coupled receptors called Y₁, Y₂ . . . Y₆ [Tatemoto et al Nature, 296, 1982, p. 659; Thorsell et al Neuropeptides, 36, 2002, p. 182; Redrobe et al Life Sci., 71, 2002, p. 2921; Silva et al. Clin. Chim. Acta, 326, 2002, p. 3; Michel et al, Pharmacol. Rev, 50, 1998, p. 143]. It is present in the central nervous system and autonomic nervous system (sympathetic fibres in which its distribution follows that of noradrenalin) [Grundemar et al Gen. Pharmacol., 24, 1993, p. 785; Lundberg et al Acta Physil. Scand., 116, 1982, p. 477; McDermott et al Cardiovasc. Res., 27, 1993, p. 893; Chronwall et al Neuroscience, 15, 1985, p. 1159]. Obese mice produce this neuropeptide in excess, and it appears to have an opposite action to leptin. For example the injection of leptin reduces NPY production. Its release in the brain is inhibited by leptin and insulin, and increased by glucocorticoids. The most notable effect of NPY is its governing of eating behaviour, in particular by stimulating the appetite via hypothalamic effect. It also reduces thermogenesis of adipocytes, inhibits lipolysis and promotes obesity. NPY has an anxiolytic and sedative effect, an antinociceptive effect (analgesic). It also appears to play a role in the central regulation of blood pressure since, when injected into certain areas of an animal brain, it causes hypotension and bradycardia. NPY has also been described as inhibiting the release of some mediators such as glutamate for example. Its chief described peripheral effect is vasoconstriction. It is also described as having digestive antisecretory effects [Mungani et al Drugs, 52, 1996, p. 371; Schwartz et al Nature, 404, 2000, p. 661; Kanatani et al Drugs of The Future, 27, 2002, p. 589; Franco-Cereceda et al Eur. J. Pharmacol., 349, 1998, p. 1].

Therefore the search for antagonists of NPY receptors has been developed in recent years, in particular for their application in the treatment of obesity [Parker et al Eur. J. Pharmacol, 440, 2002, p. 173].

The applicant has discovered a family of compounds having an affinity for NPY receptors, the NPY Y1 receptor in particular. More specifically, the compounds described below show antagonist activity against NYP receptors, and against Y1 in particular. In this respect, they may be of major interest in the treatment of various diseases and disorders, in particular for the treatment and/or prevention of obesity or metabolic disorders.

One first subject-matter of the invention concerns compounds having the following general formula (I):

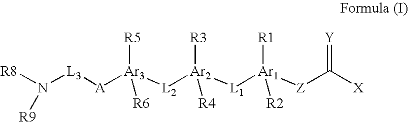

in which:
X represents a N—(C1-C6)alkylamino group, optionally substituted by a (C1-C3)alkoxy (C1-C3)alkyl group; a N,N—(C1-C6)dialkylamino(C1-C3)alkyl group,
or X is a group of hydrazino type, as represented below:

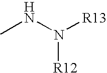

in which R12 and R13, the same or different, represent a hydrogen atom or a (C1-C6)alkyl radical, or else R12 and R13 may, with the nitrogen atom to which they are attached, form a nitrogen-containing heterocycle such as aziridine, azetidine, pyrrolidine, piperidine, homopiperidine,
   Z represents the oxygen atom or a —NH— radical,
   Ar1 represents a phenyl,
   Y represents the oxygen or sulfur atom,
   Or else Y represents the nitrogen atom and in this case, together with Z and the phenyl to which Z is attached, it forms a heterocycle such benzimidazole or benzoxazole,
   R1 and R2, the same or different, represent a hydrogen atom; a halogen atom; a hydroxyl group; a (C1-C6)alkyl, (C1-C6)alkoxy, hydroxy(C1-C3)alkyl, (C1-C6)alkoxy(C1-C3)alkyl, (C1-C3)alkoxy(C2-C3)alkoxy, hydroxy(C2-C3)alkoxy, amino(C2-C3)alkoxy, N—(C1,C3)alkylamino(C2-C3)alkoxy, N,N—(C1-C3)dialkylamino(C2-C3)alkoxy, trifluoromethyl, trifluoromethoxy, aminocarbonyl, N—(C1-C6)alkylaminocarbonyl, N,N—(C1-C6)dialkylaminocarbonyl, aminocarbonyl(C1-C3)alkyl, N—(C1-C6)alkylaminocarbonyl(C1-C3)alkyl, N,N—(C1-C6)dialkylaminocarbonyl(C1-C3)alkyl, (C1-C6)alkoxycarbonyl, or (C1-C6)alkoxycarbonyl(C1-C3)alkyl radical,
   L1 represents the oxygen atom, sulfur atom or a (C1-C3)alkylene group,
   Ar2 represents an aryl, heteroaryl or heterocyclic group such as phenyl, thiazole, indole, benzofurane, benzoxazole, benzimidazole, 2,3-dihydrobenzofurane, or 3H-quinazolin-4-one,
   R3 and R4, the same or different, represent a hydrogen atom; a halogen atom; a hydroxyl group; a (C1-C6)alkyl, (C1-C6)alkoxy, hydroxy(C1-C3)alkyl, (C1-C6)alkoxy(C1-C3)alkyl, (C1-C3)alkoxy(C2-C3)alkoxy, hydroxy(C2-C3)alkoxy, amino(C2-C3)alkoxy, N—(C1-C3)alkylamino(C2-C3)alkoxy, N,N—(C1-C3)dialkylamino(C2-C3)alkoxy, trifluoromethyl or trifluoromethoxy radical,
   R1 and R3 may also together, and with Ar1, Ar2 and L1, form a tricycle and in this case R1 and R3 together represent a (C1-C3)alkylene group, with L1 particularly representing an oxygen or sulfur atom and Ar2 a phenyl,
   When Ar2 is a phenyl or thiazole, L2 represents one of the groups below:

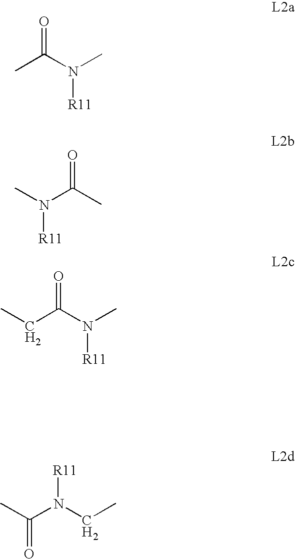

in which:
R11 represents the hydrogen atom; a (C1-C6)alkyl radical, optionally mono- or polyfluorinated, optionally substituted by a heterocycle such as tetrahydrofurane or tetrahydropyrane; a (C3-C10)cycloalkyl radical; a hydroxy(C2-C6)alkyl group; (C1-C6)alkoxy(C2-C6)alkyl group; amino(C2-C6)alkyl group; N—(C1-C6)alkylamino(C2-C6)alkyl group; N,N—(C1-C6)dialkylamino(C2-C6)alkyl group; or a heterocycle such as tetrahydrofurane or tetrahydropyrane;
For L2a, L2c and L2d, R11 may also together with Ar2, which in this case represents a phenyl group, and with the nitrogen to which it is attached, form a heterocycle such as indoline; isoindolin; tetrahydroisoquinoleine; tetrahydroquinoleine; 3,4-dihydro-2H-benzo[1,4]oxazin; 6,7,8,9-tetrahydro-5-oxa-9-aza-benzocycloheptene or 1,2,3,5-tetrahydro-benzo[e][1,4]oxazepine;
or else for L2b, R11 may also together with Ar3, which in this case represents a phenyl group, and with the nitrogen to which it is attached, form a heterocycle such as indoline; tetrahydroquinoleine; 3,4-dihydro-2H-benzo[1,4]oxazine; 6,7,8,9-tetrahydro-5-oxa-9-aza-benzocycloheptene or 1,2,3,5-tetrahydro-benzo[e][1,4]oxazepine;
Also, for L2a, L2c and L2d, R11 may together with Ar3, which in this case represents a phenyl group, and with the nitrogen to which it is attached, form a heterocycle such as 1,3-dihydro-indol-2-one; 2,3-dihydro-isoindol-1-one; 1,4-dihydro-2H-isoquinolin-3-one or 3,4-dihydro-2H-quinolin-1-one;
or else for L2b, R11 may together with Ar2, which in this case represents a phenyl group, and with the nitrogen to which it is attached form a heterocycle such as 2,3-dihydro-isoindol-1-one or 3,4-dihydro-2H-isoquinolin-1-one;
or else L2 represents a methyleneoxy or oxymethylene radical,
or else L2 represents a simple bond with Ar2 representing a phenyl, indole, benzofurane, benzoxazole, benzimidazole, or 3H-quinazolinone group,
or else L2 represents a simple bond with Ar2 representing a phenyl group and Ar3 representing an indole, benzofurane, benzoxazole, benzimidazole, 2,3-dihydro-benzofurane or 3H-quinazolinone group,
Ar3 represents a heteroaryl, aryl ou heterocyclic group such as phenyl, indole, benzofurane, benzoxazole, benzimidazole, 2,3-dihydro-benzofurane, or piperidine, Ar3 and Ar2 not being able to be heteroaryl or heterocyclic groups simultaneously when L2 is a simple bond.
R5 and R6, the same or different, represent a hydrogen atom, a halogen atom, a hydroxyl or trifluoromethyl group; a (C1-C6)alkyl, (C1-C6)alkoxy, hydroxy(C1-C3)alkyl, (C1-C6)alkoxy(C1-C3)alkyl, (C1-C3)alkylcarbonyl, (C1-C3)alkoxy(C2-C3)alkoxy, hydroxy(C2-C3)alkoxy, amino(C2-C3)alkoxy, N—(C1-C3)alkylamino(C2-C3)alkoxy or N,N—(C1-C3)dialkylamino(C2-C3)alkoxy radical,
A represents a simple bond, an oxygen atom; a (C1-C3)alkylene, (C2-C3)alkylidene, (C1-C3)alkylenoxy or oxy(C1-C3)alkylene group,
Or else A represents one of the groups described below:

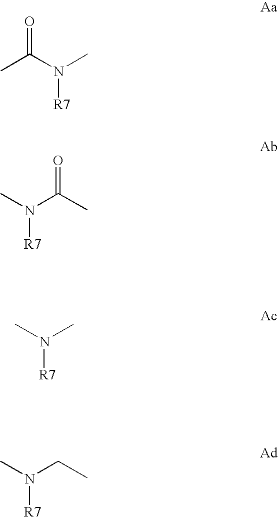

in which:
   R7 represents a hydrogen atom; a (C1-C6)alkyl or (C1-C6)alkylcarbonyl group;
   Also R7, together with L3 and the nitrogen atom to which R7 is attached, may form a nitrogen-containing heterocycle such as piperidine, pyrrolidine, homopiperidine, pyrrolidin-2-one, piperidin-2-one, azepan-2-one;
   R7 may optionally, together with Ar3 which in this case represents a phenyl group, and with the nitrogen to which it is attached, form a heterocycle such as indoline, tetrahydroquinoleine, 2,3-dihydro-isoindol-1-one or 3,4-dihydro-2H-isoquinolin-1-one,
L3 represents a (C1-C6)alkylene, (C3-C8)cycloalkylene, N—(C2-C6)alkyleneamino, (C2-C6)alkylidene, (C3-C8)cycloalkylidene, bicyclo or polycyclo(C6-C12)alkylene, bicyclo or polycyclo(C6-C12)alkylidene radical, L3 not being able to be a methylene radical if it is directly attached both to an oxygen atom and to a nitrogen atom or to two nitrogen atoms, the above-cited radicals optionally being substituted by one or more fluorine atoms, by one or more (C1-C3)alkyl, (C1-C3)alkoxy, hydroxy, hydroxy(C1-C3)alkyl or (C1-C3)alkoxy groups,
L3 may possibly, together with A and Ar3, form an oxygen-containing heterocycle such as 2,3-dihydrobenzofurane, benzofurane or chromane,
R8 and R9, the same or different, represent a hydrogen atom; a (C1-C6)alkyl group, optionally substituted by a phenyl radical, by a saturated nitrogen- or oxygen-containing heterocycle such as tetrahydropyran-3 or 4-yl, piperidin-3 or -4-yl, pyrrolidin-3-yl or morpholin-1-yl; a (C1-C6)alkoxy(C2-C6)alkyl group; (C3-C8)cycloalkyl group; (C3-C8)cycloalkyl(C1-C4)alkyl group; a saturated nitrogen- or oxygen-containing saturated heterocycle such as tetrahydropyran-3 or -4-yl, piperidin-3 ou -4-yl, pyrrolidin-3-yl; an amino, N—(C1-C6)alkylamino, N,N—(C1,C6)dialkylamino, amino(C2-C6)alkyl, N—(C1-C4)alkylamino(C2-C6)alkyl, N,N—(C1-C4)dialkylamino(C2-C6)alkyl, N,N—(C1-C4)dialkylamino(C1-C6)alkylcarbonyl, tetrahydropyran-4-yl-amino(C2-C6)alkyl, hydroxy(C2-C6)alkyl, (C1-C4)alkoxy(C2-C6)alkyl, hydroxycarbonyl(C1-C3)alkyl, (C1-C6)alkoxycarbonyl(C1-C3)alkyl or (C1-C3)alkylcarbonyloxy(C2-C6)alkyl radical, the above-cited groups possibly being substituted by one or more fluorine atoms,
R8 and R9, together and with the nitrogen atom to which they are attached, may form a mono- or polycyclic nitrogen-containing heterocycle such as aziridine, azetidine, pyrrolidine, piperidine, piperazine, homopiperazine, [1,5]diazocane, homopiperidine, morpholine, 2,7-diaza-spiro[4.4]nonane, octahydro-pyrrolo[3,4-c]pyrrole, octahydro-pyrrolo[3,2-b]pyrrole, optionally substituted by one or more fluorine atoms, by one or more hydroxyl, hydroxy(C1-C6)alkyl, (C1-C6)alkyl, (C1-C6)alkoxy, amino(C1-C6)alkyl, N—(C1-C4)alkylamino(C1-C6)alkyl, N,N—(C1-C4)dialkylamino(C1-C6)alkyl, (C1-C4)alkoxy(C1-C6)alkyl, hydroxycarbonyl(C1-C3)alkyl, (C1-C6)alkoxycarbonyl(C1-C3)alkyl, (C1-C3)alkylcarbonyloxy(C1-C6)alkyl or mono or polyfluoro(C1-C6)alkyl radicals,
R8 and/or R9, together with L3 and with the nitrogen atom to which they are attached, may form a mono- or polycyclic, saturated or unsaturated nitrogen-containing heterocycle such as pyrrolidine, piperidine, homopiperidine, 8-azabicyclo[3.2.1]octane, 2-aza-bicyclo[2.2.2]octane, 2-aza-bicyclo[2.2.1]heptane, 7-aza-bicyclo[2.2.1]heptane, 1,2,3,6-tetrahydro pyridine, optionally substituted by one or more fluorine atoms, by one or more hydroxyl, hydroxy(C1-C6)alkyl, (C1-C6)alkyl, (C1-C6)alkoxy, amino(C1-C6)alkyl, N—(C1-C4)alkylamino(C1-C6)alkyl, N,N—(C1-C4)dialkylamino(C1-C6)alkyl, (C1-C4)alkoxy(C1-C6)alkyl, hydroxycarbonyl(C1-C3)alkyl, (C1-C6)alkoxycarbonyl(C1-C3)alkyl, (C1-C3)alkylcarbonyloxy(C1-C6)alkyl or mono- or polyfluoro(C1-C6)alkyl radicals; for the particular case in which L3, together with A and Ar3, forms an oxygen-containing heterocycle and at the same time R8 and/or R9, together with L3 and with the nitrogen atom to which they are attached, form a nitrogen-containing heterocycle, the whole forms a polycycle such as 1,2,3,4-tetrahydro-benzo[4,5]furo[3,2-c]pyridine or 1,2,3,4,4a,9b-hexahydro-benzo[4,5]furo[3,2-c]pyridine, or else a polycycle such as described below:

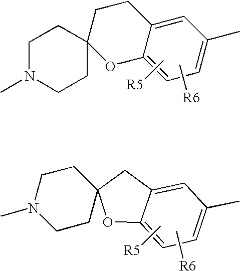

when A represents one of the Aa, Ab, Ac or Ad groups, R8 and/or R9 together with R7, L3 and the nitrogen atom to which R8 and R9 are attached, may possibly form a mono- or polycyclic nitrogen-containing heterocycle such as piperazine, homopiperazine, [1,5]diazocane, 2,7-diaza-spiro[4.4]nonane, octahydro-pyrrolo[3,4-c]pyrrole, octahydro-pyrrolo[3,2-b]pyrrole, piperazin-2-one, [1,4]diazepan-5-ou-2-one, [1,5]diazocan-2-one,
the nitrogen atom attached to R8 and R9 possibly being in quaternary ammonium form, in which case it can be in the following form:

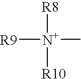

R8 and R9 being as defined above, in particular they represent a (C1-C6)alkyl group, and R10 represents a (C1-C6)alkyl group,
and their pharmaceutically acceptable salts, their solvates and hydrates, optical and geometric isomers or their mixtures.

According to the present invention, the term <<alkyl>> designates a saturated hydrocarbon monovalent radical, whether straight or branched.

By (C1-C3)alkyl; (C1-C4)alkyl; (C2-C6)alkyl and (C1-C6)alkyl, is meant an alkyl radical containing 1 to 3; 1 to 4; 2 to 6 and respectively 1 to 6 carbon atoms. Particular mention may be made of methyl, ethyl, propyl, isopropyl, butyl, isobutyl, sec-butyl, tert-butyl, 1-ethyl-propyl, pentyl, neopentyl or n-hexyl radicals.

By hydroxyalkyl, is meant a hydroxyl group joined to the remainder of the molecule by an alkyl radical such as defined above.

The mono or polyfluoro(C1-C6)alkyl groups are alkyl radicals carrying one or more fluorine atoms. Particular mention may be made of the perfluoroalkyl radicals, such as perfluoromethyl, or the 4-fluoro-butyl, 4,4,4-trifluoro-butyl, 3,3,3-trifluoro-propyl or 2,2,2-trifluoro-ethyl radicals.

By aminoalkyl, is meant a NH₂— group joined to the remainder of the molecule by an alkyl radical such as defined above.

The term <<tetrahydropyran-4-yl-aminoalkyl>> refers to a tetrahydropyran-4-yl group joined to the remainder of the molecule by an aminoalkyl radical such as defined above.

In the meaning of the invention, the term <<cycloalkyl>> designates an alkyl group of 3 to 10 carbon atoms forming a saturated monocyclic system. As examples, particular mention may be made of cyclopropyl, cyclobutyl, cyclopentyl, cyclohexyl, cycloheptyl, or norbornyl.

By (C3-C8)cycloalkyl, is meant a cycloalkyl radical containing 3 to 8 carbon atoms. By <<cycloalkyl alkyl>>, is meant a cycloalkyl group joined to the remainder of the molecule by an alkyl radical such as defined above.

The <<alkylene>> groups in the meaning of the invention are divalent groups corresponding to the alkyl groups such as defined above, by removing one hydrogen atom.

For example, the (C1-C3)alkylene and (C2-C6)alkylene groups correspond to an alkylene radical containing 1 to 3 and 2 to 6 carbon atoms respectively.

In the meaning of the invention, the term <<cycloalkylene>> designates a divalent cycloalkyl group such as defined above, by removing a hydrogen atom. By (C3-C8)cycloalkylene, is meant a cycloalkylene radical containing 3 to 8 carbon atoms.

By polycyclo (C6-C12)alkylene, is meant an alkylene radical containing 6 to 12 carbon atoms forming a saturated polycyclic system.

The <<alkylidene>> groups in the meaning of the invention are divalent groups corresponding to the alkylene groups such as defined above and containing at least one ethylene unsaturation.

By (C2-C3)alkylidene and (C2-C6)alkylidene, is meant an alkylidene radical containing 2 to 3 and 2 to 6 carbon atoms respectively.

By polycyclo(C6-C1-2)alkylidene, is meant a polycyclic alkylidene radical containing 6 to 12 carbon atoms.

The <<aryl>> groups are mono- or bicyclic aromatic hydrocarbon systems, generally a 5- or 6-membered ring, having 6 to 14 carbon atoms. Particular mention may be made of the phenyl or naphtyl radical. The <<heteroaryl>> groups are aromatic hydrocarbon systems such as defined above having in the cycle(s) at least one heteroatom, such as nitrogen, sulfur or oxygen. As heteroaryl, particular mention may be made of the pyrrole, pyrazole, imidazole, furane, oxazole, thiazole, thiadiazole, oxadiazole, indole, benzimidazole, benzoxazole, benzofurane, benzothiazole, and pyridine groups.

The term <<heterocycle>> designates mono-, bi- or polycyclic hydrocarbon systems, whether saturated or unsaturated, having in the cycle(s) at least one heteroatom such as nitrogen, sulfur or oxygen. They may or may not be aromatic. They are preferably non-aromatic. As heterocycle, particular mention may be made of the following groups: piperidine, pyrane, dioxane, piperazine, pyrrolidine, morpholine, homopiperazine, homopiperidine, thiomorpholine, [1,5]diazocane, pyrrolidin-2-one, piperidin-2-one, azepan-2-one, piperazin-2-one, [1,4]diazepan-5-one, [1,4]diazepan-2-one, [1,5]diazocane-2-one, 2,7-diaza-spiro[4.4]nonane, octahydro-pyrrolo[3,4-c]pyrrole, octahydro-pyrrolo[3,2-b]pyrrole, 8-azabicyclo[3.2.1]octane, 2-aza-bicyclo [2.2.2]octane, 2-aza-bicyclo[2.2.1]heptane, 7-aza-bicyclo[2.2.1]heptane, 2,3-dihydro-benzofurane, 1,2,3,6-tetrahydropyridine, indoline, isoindoline, tetrahydroisoquinoleine, tetrahydroquinoleine, 3,4-dihydro-2H-benzo[1,4]oxazine, 6,7,8,9-tetrahydro-5-oxa-9-aza-benzocycloheptene, 1,2,3,5-tetrahydro-benzo[e][1,4]oxazepine, 1,3-dihydro-indol-2-one, 2,3-dihydro-isoindol-1-one, 1,4-dihydro-2H-isoquinolin-3-one, 3,4-dihydro-2H-quinolin-1-one or 3H-quinazolin-4-one.

By polycycle, is meant a radical containing at least two hydrocarbon rings, aromatic or non-aromatic, saturated or unsaturated, optionally having one or more heteroatoms such as O, N or S. As polycycle, particular mention may be made of the groups 1,2,3,4-tetrahydro-benzo[4,5]furo[3,2-c]pyridine or 1,2,3,4,4a,9b-hexahydro-benzo[4,5]furo[3,2-c]pyridine, or else the groups described below:

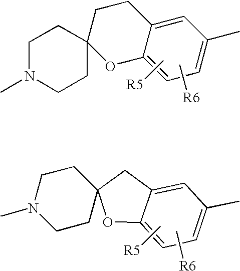

The <<alkoxy>> groups correspond to the alkyl groups defined above and joined to the remainder of the molecule via an oxygen atom. As examples, particular mention may be made of the following radicals: methoxy, ethoxy, propoxy, isopropoxy, n-butoxy, sec-butoxy, tert-butoxy, pentoxy, 2-pentoxy, isopentoxy, neopentoxy, hexoxy, 2-hexoxy, 3-hexoxy, 3-methylpentoxy.

For example, the (C1-C4)alkoxy; (C1-C6)alkoxy and (C1-C3)alkoxy groups correspond to an alkyl radical containing 1 to 4; 1 to 6 and 1 to 3 carbon atoms respectively, joined to the remainder of the molecule via an oxygen atom.

An alkoxyalkyl group corresponds to an alkyl radical interrupted by an oxygen atom.

The <<alkyleneoxy>> or <<oxyalkylene>> groups correspond to the alkylene groups as defined above and joined to the remainder of the molecule in particular via an oxygen atom.

By (C1-C3)alkyleneoxy or oxy(C1-C3)alkylene, is meant an alkylene radical containing 1 to 3 carbon atoms, joined to the remainder of the molecule in particular via an oxygen atom.

The term <<alkylcarbonyl>> refers to alkyl groups such as defined above and joined to the remainder of the molecules via a —C═O— (carbonyl) group.

By (C1-C3)alkylcarbonyl and (C1-C6)alkylcarbonyl, is meant alkyl radicals such as defined above and containing 1 to 3 and 1 to 6 carbon atoms respectively, joined to the remainder of the molecule via a —C═O— (carbonyl) group.

By hydroxycarbonylalkyl, is meant a hydroxycarbonyl (carboxyl) —COOH group, joined to the remainder of the molecule via an alkyl such as defined above.

The terme <<alkoxycarbonyl>> refers to alkoxy groups such as defined above and joined to the remainder of the molecule via a —C═O— (carbonyl) group.

By (C1-C6)alkoxycarbonyl is meant alkoxy groups such as defined above, containing 1 to 6 carbon atoms, joined to the remainder of the molecule via a —C═O— (carbonyl) group.

By <<alkoxycarbonyl alkyl>>, is meant an alkoxycarbonyl group, joined to the remainder of the molecule by an alkyl radical such as defined above.

The term <<alkylcarbonyloxy alkyl>> refers to an alkyl radical such as defined above, interrupted by a

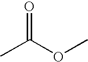

(carbonyloxy) group.

The <<N-alkylamino>> or <<N,N-dialkylamino>> groups correspond to an alkyl group or respectively to two alkyl groups such as defined above, joined to the remainder of the molecule by a nitrogen atom or amino group. An <<alkylaminoalkyl>> group corresponds to an alkyl radical interrupted by an amino group.

The <<N-alkyleneamino>> groups in the meaning of the invention are divalent groups corresponding to the N-alkylamino groups, such as defined above, by removing a hydrogen atom. For example, the N—(C2-C6)alkyleneamino groups correspond to an alkylene radical containing 2 to 6 carbon atoms, joined to the remainder of the molecule by a nitrogen atom or an amino group.

By (C1-C6)dialkylhydrazino is meant a hydrazino group of the type

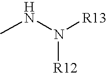

such as defined in formula (I) above, for which R12 and R13 are alkyl radicals containing 1 to 6 carbon atoms.

The <<N-alkylaminocarbonyl>> or <<N,N-dialkylaminocarbonyl>> groups correspond to the alkylamino or dialkylamino groups such as defined above, joined to the remainder of the molecule via a —C═O— (carbonyl) group.

The term <<alkylaminocarbonyl alkyl>> refers to an alkylaminocarbonyl group such as defined above, joined to the remainder of the molecule via an alkyl.

An aminocarbonyl group corresponds to a NH₂— amine group, joined to the remainder of the molecule by a —C═O— (carbonyl) group.

The terme <<aminocarbonyl alkyl>> refers to an aminocarbonyl group such as defined above, joined to the remainder of the molecule via an alkyl.

An alkylaminoalkylcarbonyl group corresponds to an alkylradical interrupted by an amino group and joined to the remainder of the molecule by a —C═O— (carbonyl) group.

An alkoxyalkoxy group is an alkoxy group joined to the remainder of the molecule via another alkoxy group.

An aminoalkoxy group is an amino group joined to the remainder of the molecule via an alkoxy group.

The N-alkylaminoalkoxy or N,N-dialkylaminoalkoxy groups correspond to the alkylamino or dialkylamino groups such as defined above, joined to the remainder of the molecule via an alkoxy radical.

By <<halogen>>, is meant a fluorine, chlorine, bromine or iodine atom.

By <(heteroatom>>, is meant an atom chosen from among O, N and S.

According to the invention, the 8-azabicyclo[3.2.1]octane group preferably has the following formula:

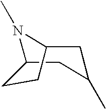

According to the invention, the 2-aza-bicyclo[2.2.2]octane group preferably has the following formula:

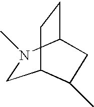

According to the invention, the 2-aza-bicyclo[2.2.1]heptane group preferably has the following formula:

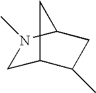

According to the invention, the 7-aza-bicyclo[2.2.1]heptane group preferably has the following formula:

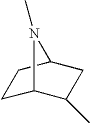

According to the invention, the 1,2,3,6-tetrahydropyridine group preferably has the following formula:

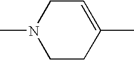

According to the invention, the [1,5]diazocane group preferably has the following formula:

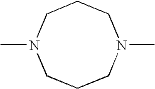

According to the invention, the 2,7-diaza-spiro[4.4]nonane group preferably has the following formula:

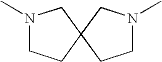

According to the invention, the octahydro-pyrrolo[3,4-c]pyrrole group preferably has the following formula:

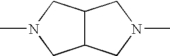

According to the invention, the octahydro-pyrrolo[3,2-b]pyrrole group preferably has the following formula:

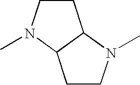

According to the invention, the azepan-2-one group preferably has the following formula:

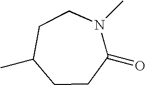

According to the invention, the [1,4]diazepan-5-one group preferably has the following formula:

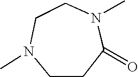

According to the invention, the [1,4]diazepan-2-one group preferably has the following formula:

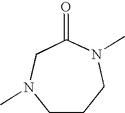

According to the invention, the [1,5]diazocan-2-one group preferably has the following formula:

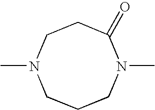

According to the invention, the tetrahydrofurane group preferably has one of the following formulas:

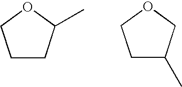

According to the invention, the tetrahydropyrane group preferably has one of the following formulas:

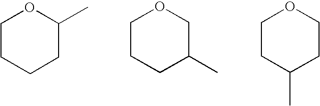

According to the invention, the thiazole group preferably has the following formula:

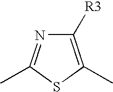

According to the invention, the indoline group preferably has one of the following formulas:

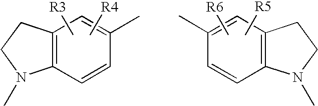

According to the invention, the isoindoline group preferably has one of the following formulas:

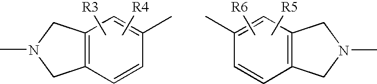

According to the invention, the tetrahydroquinoleine group preferably has one of the following formulas:

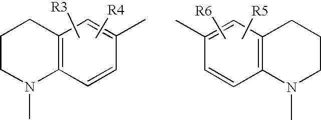

According to the invention, the tetrahydroisoquinoleine group preferably has one of the following formulas:

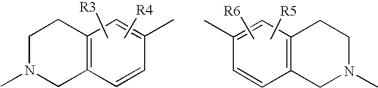

According to the invention, the 3,4-dihydro-2H-benzo[1,4]oxazine group preferably has one of the following formulas:

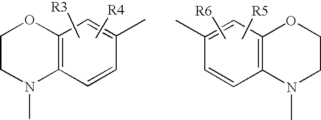

According to the invention, the 6,7,8,9-tetrahydro-5-oxa-9-aza-benzocycloheptene group preferably has one of the following formulas:

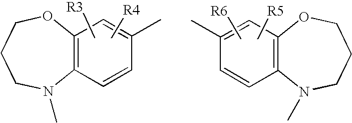

According to the invention, the 1,2,3,5-tetrahydro-benzo[e][1,4]oxazepine group preferably has one of the following formulas:

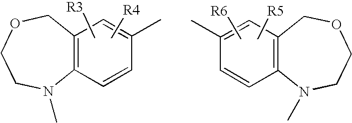

According to the invention, the 1,3-dihydro-indol-2-one group preferably has the following formula:

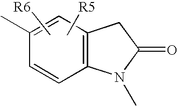

According to the invention, the 2,3-dihydro-isoindol-1-one group preferably has one of the following formulas:

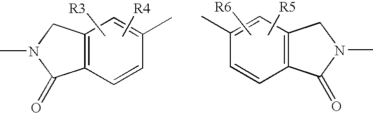

According to the invention, the 3,4-dihydro-2H-isoquinolin-1-one group preferably has one of the following formulas:

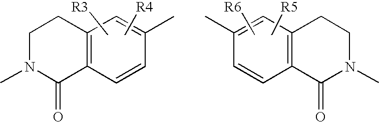

According to the invention, the 1,4-dihydro-2H-isoquinolin-3-one group preferably has the following formula:

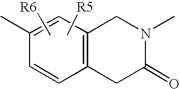

According to the invention, the 3H-quinazolinone-4-one group preferably has one of he following formulas:

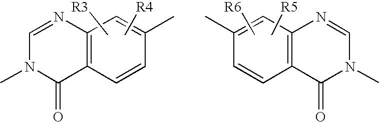

According to the invention, the indole group preferably has one of the following formulas:

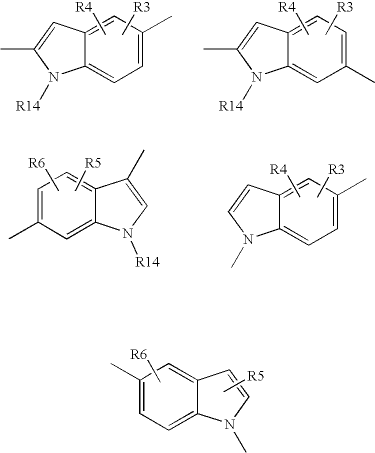

in which R14 is a hydrogen atom, a (C1-C6)alkyl or (C1-C3)alkoxy(C2-C6)alkyl radical and R3 to R6 being defined as previously. According to the invention, the benzimidazole group preferably has one of the following formulas:

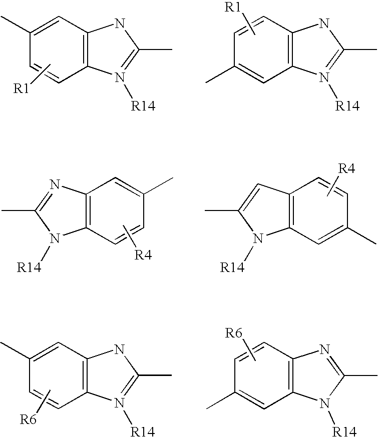

in which R14 is a hydrogen atom, a (C1-C6)alkyl or (C1-C3)alkoxy(C2-C6)alkyl radical, and R1, R4, and R6 being as previously defined.

According to the invention, the benzoxazole group preferably has one of the following formulas:

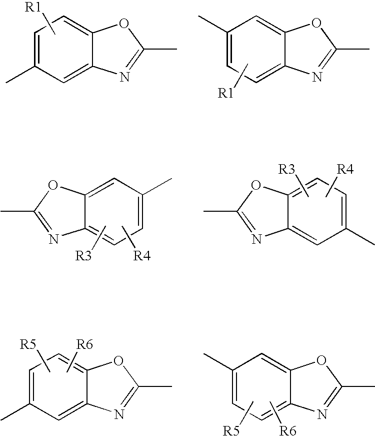

According to the invention, the benzofurane group preferably has one of the following formulas:

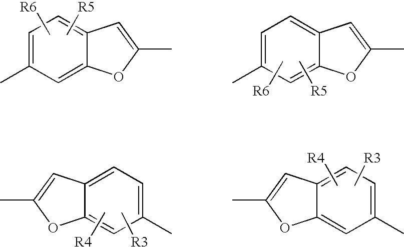

According to the invention, the 2,3-dihydro-benzofurane group preferably has one of the following formulas:

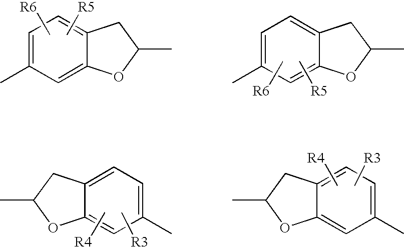

According to the invention, the chromane group preferably has the following formula:

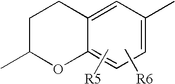

According to the invention, the 1,2,3,4-tetrahydro-benzo[4,5]furo[3,2-c]pyridine group preferably has the following formula:

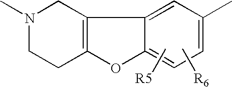

According to the invention, the 1,2,3,4,4a,9b-hexahydro-benzo[4,5]furo[3,2-c]pyridine group preferably has the following formula:

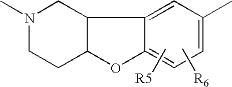

The groups R1 to R6 and R14 being as defined above.

The above-identified formulas defining certain particular groups of the invention can be read from left to right and from right to left.

According to one particular aspect of the invention, the preferred compounds of the invention are compounds of formula (I) such as afore-defined, wherein at least one of the groups R8 and R9 is different from the hydrogen atom.

According to another particular aspect of the invention, a family of preferred compounds corresponds to compounds of formula (I) above, wherein R1 is such as defined above and R2 is a hydrogen atom. In particular, R1 represents a hydrogen atom; a halogen atom; a (C1-C6)alkyl, (C1-C6)alkoxy, hydroxy(C1-C3)alkyl, (C1-C6)alkoxy(C1-C3)alkyl, trifluoromethyl, N—(C1-C6)alkylaminocarbonyl, N—(C1-C6)alkylaminocarbonyl(C1-C3)alkyl, or (C1-C6)alkoxycarbonyl radical.

According to another particular aspect of the invention, a family of preferred compounds corresponds to compounds of formula (I) above, wherein R1 and R2, such as defined above, are simultaneously different from the hydrogen atom. In particular, they may be a halogen atom, preferably fluorine; a (C1-C6)alkyl, (C1-C6)alkoxy, hydroxy(C1-C3)alkyl, (C1-C6)alkoxy(C1-C3)alkyl or N,N—(C1-C3)dialkylamino(C2-C3)alkoxy radical.

According to another particular aspect of the invention, a family of preferred compounds corresponds to compounds of formula (I) wherein Y represents an oxygen atom, Z represents a —NH— radical and advantageously X represents a N—(C1-C6)alkylamino group, optionally substituted by a (C1-C3)alkoxy(C1-C3)alkyl group.

According to one particular aspect of the invention, a family of preferred compounds corresponds to compounds of formula (I) above, wherein L2 is an amide bond of L2a type. This family can be represented by the following formula (I′):

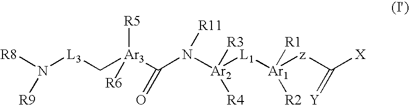

According to one particularly preferred variant, the compounds of the invention are compounds of formula (I′) above, wherein A is an oxygen atom, Ar1 and Ar3 are phenyl radicals, Ar2 is a thiazole or a phenyl and X, Y, Z, L1, L3, R1 to R9 and R11 are such as defined in general formula (I) above; This family of compounds is represented by the following formula (II):

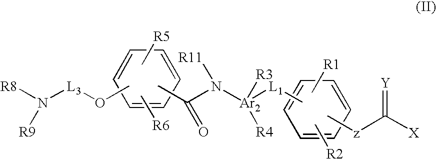

According to one preferred variant (IIa), L1 is an oxygen, Ar1 and Ar3 are advantageously 3- or 4-phenyl radicals and Ar2 is a thiazole, according to the following formula (IIa):

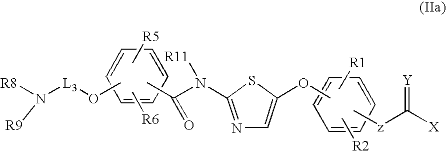

Depending upon the different variants, the compounds of structure (IIa) advantageously have the following characteristics:
Ar1 and Ar3 are 4-phenyl radicals,
   Or else
Ar1 is the 4-phenyl radical and Ar3 is the 3-phenyl radical.

Further advantageously, in formula (IIa):
X represents a N—(C1-C6)alkylamino group, optionally substituted by a (C1-C3)alkoxy(C1-C3)alkyl group, preferably isopropylamino and 1-ethyl-propylamino, or a (C1-C6)dialkylhydrazino group such as defined in formula (I) above, preferably 2,2-dimethylhydrazino, and/or
Y is an oxygen, and/or
Z is a —NH— radical, and/or
R11 represents the hydrogen atom or a (C1-C6)alkyl radical, and/or
L3 is a (C2-C6)alkylene group, and/or
R8 and R9, such as defined in formula (I) above, together and with the nitrogen atom to which they are attached, form a nitrogen-containing heterocycle, preferably piperidine,
Or else R9, together with L3 and with the nitrogen atom to which it is attached, forms a nitrogen-containing heterocycle, preferably piperidine or pyrrolidine,
R1, R2, R5, R6 and R8 are such as defined in formula (I) above.

According to one preferred variant, (IIb), Ar1, Ar2 and Ar3 advantageously represent 3- or 4-phenyl radicals. Formula (IIb) can be represented as follows:

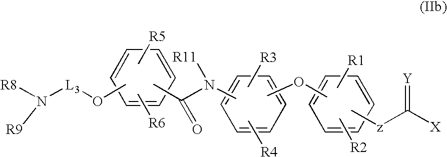

Depending upon the different variants, the compounds of the sub-family (IIb) advantageously have the following characteristics:
Ar1, Ar2 and Ar3 are 4-phenyl radicals,
   Or else:
Ar1 is the 4-phenyl radical and Ar2 and Ar3 are 3-phenyl radicals,
   Or else:
Ar1 and Ar2 are 4-phenyl radicals and Ar3 is the 3-phenyl radical,
   Or else:
Ar1 and Ar3 are 3-phenyl radicals and Ar2 is the 4-phenyl radical,
   Or else:
Ar1 is the 3-phenyl radical and Ar2 and Ar3 are 4-phenyl radicals,
   Or else:
Ar1 and Ar3 are 4-phenyl radicals and Ar2 is the 3-phenyl radical.

Further advantageously, in formula (IIb):
X represents a N—(C1-C6)alkylamino group, optionally substituted by a (C1-C3)alkoxy(C1-C3)alkyl group, preferably isopropylamino, 1-ethyl-propylamino and 1-methoxymethyl-propylamino, or a (C1-C6)dialkylhydrazino group such as defined in formula (I) above, preferably 2,2-dimethylhydrazino, and
Y, Z, L3, R1 to R11 are such as defined in formula (I) above.

According to one preferred variant, the compounds of sub-family (IIb) have the following characteristics:
X represents a N—(C1-C6)alkylamino group, optionally substituted by a (C1-C3)alkoxy(C1-C3)alkyl group, preferably isopropylamino, 1-ethyl-propylamino and 1-methoxymethyl-propylamino, or a (C1-C6)dialkylhydrazino group such as defined in formula (I) above, preferably 2,2-dimethylhydrazino, and/or
Y is the oxygen atom, and/or
Z is a —NH— radical, and/or
R11 represents the hydrogen atom; a (C1-C6)alkyl radical, optionally mono or polyfluorinated, optionally substituted by a heterocycle such as tetrahydrofurane or tetrahydropyrane; a (C3-C10)cycloalkyl radical; a hydroxy(C2-C6)alkyl group; (C1-C6)alkoxy(C2-C6)alkyl group; amino(C2-C6)alkyl group; N—(C1-C6)alkylamino(C2-C6)alkyl group; N,N—(C1-C6)dialkylamino(C2-C6)alkyl group; a heterocycle such as tetrahydrofurane or tetrahydropyrane,
Or else R11, together with Ar2 and with the nitrogen to which it is attached, forms a heterocycle, preferably indoline, 3,4-dihydro-2H-benzo[1,4]oxazine, 6,7,8,9-tetrahydro-5-oxa-9-aza-benzocycloheptene or 1,2,3,5-tetrahydro-benzo[e][1,4]oxazepine, and in this case the group

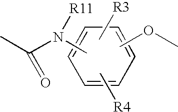

in formula (IIb) above, preferably represents:

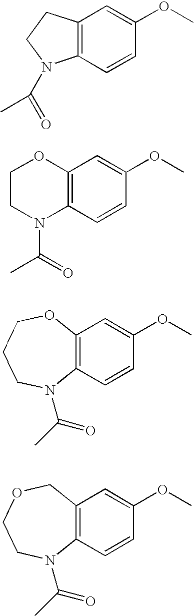

and/or
R9, together with L3 and with the nitrogen atom to which it is attached, forms a nitrogen-containing heterocycle, and in this case the group:

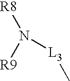

preferably represents a pyrrolidin-3-yl, piperidin-3 or 4-yl, homopiperidin-4-yl radical, optionally substituted by one or more fluorine atoms, by one or more hydroxyl, hydroxy(C1-C6)alkyl, (C1-C6)alkyl, (C1-C6)alkoxy, amino(C1-C6)alkyl, N—(C1-C4)alkylamino(C1-C6)alkyl, N,N—(C1-C4)dialkylamino(C1-C6)alkyl, (C1-C4)alkoxy(C1-C6)alkyl, hydroxycarbonyl(C1-C3)alkyl, (C1-C6)alkoxycarbonyl(C1-C3)alkyl, (C1-C3)alkylcarbonyloxy(C1-C6)alkyl, or mono- or polyfluoro(C1-C6)alkyl radicals,
or else the group:

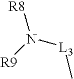

preferably represents:

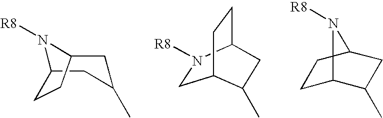

R1 to R6 and R8 are such as defined in formula (I) above,

According to another preferred variant, the compounds of sub-family (IIb) have the following characteristics:
X represents a (C1-C6)alkylamino group, optionally substituted by a (C1-C3)alkoxy(C1-C3)alkyl group, preferably isopropylamino and 1-ethyl-propylamino, or a (C1-C6)dialkylhydrazino group such as defined in formula (I) above, preferably 2,2-dimethylhydrazino, and/or
Y is an oxygen, and/or
Z is a —NH— radical, and/or
R11 represents the hydrogen atom or a (C1-C6)alkyl radical, and/or
L3 is a (C2-C6)alkylene group, and/or
R8 and R9 together and with the nitrogen atom to which they are attached, form a nitrogen-containing heterocycle, preferably piperidine, optionally substituted by a hydroxyl radical, and/or
R1 to R6 are such as defined in formula (I) above.

According to another variant, the compounds of type (II) correspond to following formula (IIc):

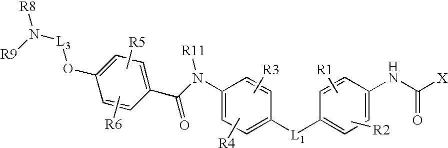

in which:
X represents a (C1-C6)alkylamino group, optionally substituted by a (C1-C3)alkoxy(C1-C3)alkyl group, preferably isopropylamino and 1-ethyl-propylamino, and/or
L1 is a sulfur atom or a —CH₂— methylene radical, and/or
R11 represents the hydrogen atom or a (C1-C6)alkyl radical, and/or
L3 is a (C2-C6)alkylene group, and/or
R8 and R9, such as defined in formula (I) above, together and with the nitrogen atom to which they are attached, form a nitrogen-containing heterocycle, preferably piperidine,
Or else R9, together with L3 and with the nitrogen atom to which it is attached, forms a nitrogen-containing heterocycle, preferably piperidine,
R1 to R6 are such as defined in formula (I) above.

According to another particular aspect of the invention, a sub-family of preferred compounds (III) corresponds to compounds of formula (I′) above, in which A represents a simple bond, an oxygen atom or a (C1-C3)alkylene, (C2-C3)alkylidene, (C1-C3)alkylenoxy or oxy(C1-C3)alkylene group, and X, Y, Z, L1, L3, Ar2, Ar3, R1 to R11 are such as defined in formula (I) above.

Formula (III) can be represented as follows:

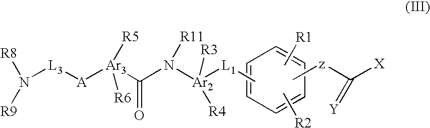

According to the preferred variant (IIa), L1 is an oxygen atom, Ar1 and Ar3 are phenyl radicals, preferably 4-phenyl and Ar2 is a thiazole. Formula (IIa) can be represented as follows:

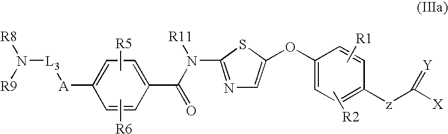

in which the group:
preferably represents:

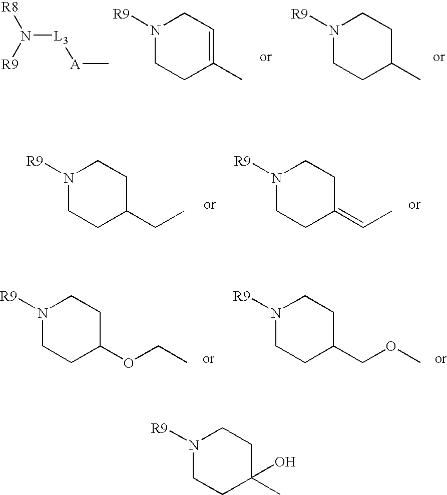

Even further advantageously, in formula (IIIa):
X represents a (C1-C6)alkylamino group, optionally substituted by a (C1-C3)alkoxy(C1-C3)alkyl group, preferably isopropylamino and 1-ethyl-propylamino, or a (C1-C6)dialkylhydrazino group such as defined in formula (I) above, preferably 2,2-dimethylhydrazino, and/or
Y is an oxygen, and/or
Z is the —NH— radical, and/or
the group:

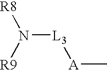

is such as defined in formula (IIIa) above,
R1, R2, R5, R6 and R11 are such as defined in formula (I) above.

According to preferred embodiment (IIIb), L1 is an oxygen, Ar2 and Ar3 are phenyl radicals, preferably 4-phenyl, and the group:

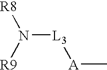

is such as defined in formula (IIIa) above.

Formula (IIIb) can be represented as follows:

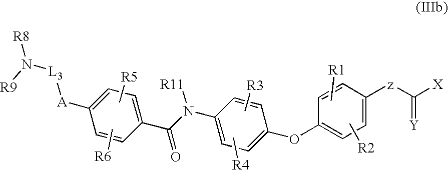

Further advantageously, in formula (IIIb):
X represents a (C1-C6)alkylamino group, optionally substituted by a (C1-C3)alkoxy(C1-C3)alkyl group, preferably isopropylamino and 1-ethyl-propylamino, or a (C1-C6)dialkylhydrazino group such as defined in formula (I) above, preferably 2,2-dimethylhydrazino, and/or
Y is an oxygen, and/or
Z is the —NH— radical, and/or
the group:

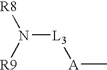

is such as defined in formula (IIIa) above,
R1 to R6 and R11 are such as defined in formula (I) above.

According to preferred variant (IIIc), the compounds of type (III) have the following characteristics:
A represents a simple bond,
Ar2 is a phenyl radical, preferably 4-phenyl, or thiazole, and/or
Ar3 is an indole, benzimidazole or benzofurane group,
the group:

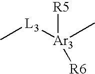

in formula (IIIc) below preferably represents:

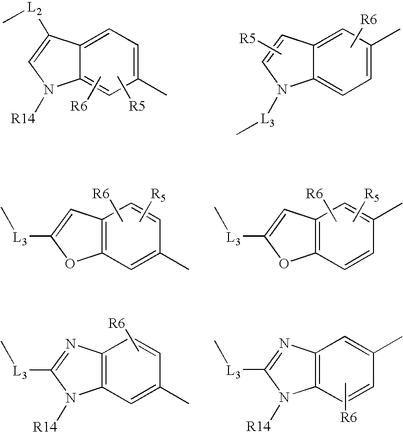

X, Y, Z, L1, L3, R1 to R11 and R14 are such as defined in formula (I) above.

Formula (IIIc) can be represented as follows:

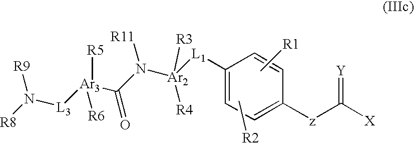

According to one preferred variant, the compounds of sub-family (IIIc) have the following characteristics:
X represents a (C1-C6)alkylamino group, optionally substituted by a (C1-C3)alkoxy(C1-C3)alkyl group, preferably isopropylamino and 1-ethyl-propylamino or a (C1-C6)dialkylhydrazino group such as defined in formula (I) above, preferably 2,2-dimethylhydrazino, and/or
Y is the oxygen atom, and/or
Z is a —NH— radical, and/or
L1 is the oxygen atom, and/or
L3 is a (C1-C6)alkylene group or a (C3-C8)cycloalkylene group, and/or
R8 and R9 such as defined in formula (I) above, together and with the nitrogen atom to which they are attached, form a nitrogen-containing heterocycle, preferably piperidine,
Or else R9, together with L3 and the nitrogen atom to which it is attached, forms a nitrogen-containing heterocycle, preferably piperidine,
R1 to R6 and R11 are such as defined in formula (I) above and R14 is a hydrogen atom, a (C1-C6) alkyl or (C1-C3)alkoxy(C2-C6)alkyl radical.

According to preferred variant (IIId), the compounds of type (III) have the following characteristics:
A represents an oxygen,
Ar2 is a phenyl radical, preferably 4-phenyl, or thiazole,
Ar3 is a piperidine,
the group:

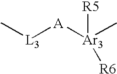

in formula (IIId) below preferably represents:

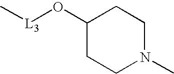

X, Y, Z, L1, L3, R1 to R4, R8, R9 and R11 are such as defined in formula (I) above.

Formula (IIId) can be represented as follows:

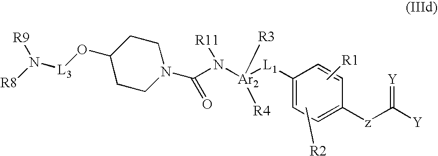

According to one preferred variant, the compounds of sub-family (IIId) have the following characteristics:
X represents a (C1-C6)alkylamino group, optionally substituted by a (C1-C3)alkoxy(C1-C3)alkyl group, preferably isopropylamino and 1-ethyl-propylamino or a (C1-C6)dialkylhydrazino group such as defined in formula (I) above, preferably 2,2-dimethylhydrazino, and/or
Y is the oxygen atom, and/or
Z is a —NH— radical, and/or
L1 is the oxygen atom, and/or
L3 is a (C1-C6)alkylene group or a (C3-C8)cycloalkylene group, and/or
R8 and R9, such as defined in formula (I) above, together and with the nitrogen atom to which they are attached, form a nitrogen-containing heterocycle, preferably piperidine,
Or else R9, together with L3 and with the nitrogen atom to which it is attached, forms a nitrogen-containing heterocycle, preferably piperidine,
R1 to R4 and R11 are such as defined in formula (I) above.

According to another particular aspect of the invention, the sub-family of compounds (IV) corresponds to compounds of formula (I′) above, in which Ar1 and Ar3 are phenyl radicals and A represents one of the groups below:

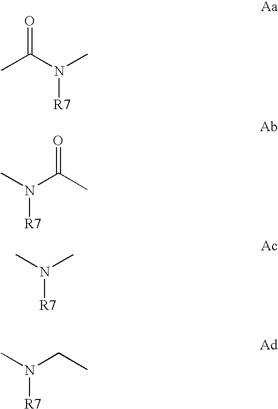

Formula (IV) can be represented as follows:

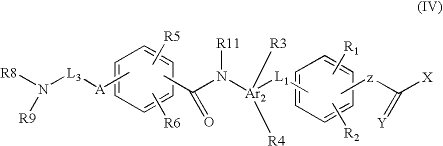

According to one preferred variant (IVa), Y is an oxygen, and/or Z is a NH group, and/or L1 is an oxygen, Ar1 and Ar3 are 4-phenyl radicals, and/or Ar2 is a thiazole and A is such as defined in formula (IV) above.

Formula (IVa) may preferably be represented as follows:

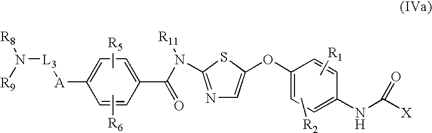

Further advantageously, in formula (IVa):
X represents a (C1-C6)alkylamino group, optionally substituted by a (C1-C3)alkoxy(C1-C3)alkyl group, preferably isopropylamino and 1-ethyl-propylamino, or a (C1-C6)dialkylhydrazino group such as defined in formula (I) above, preferably 2,2-dimethylhydrazino, and/or
R11 represents the hydrogen atom and/or a (C1-C6)alkyl radical, and/or
L3 and R1 to R9 are such as defined in formula (I) above.

According to preferred variant (IVb), Y is an oxygen, and/or Z is a NH group, and/or L1 is an oxygen, Ar1, Ar2 and Ar3 are phenyl radicals, preferably 4-phenyl and A is such as defined in formula (IV) above.

Formula (IVb) may preferably be represented as follows:

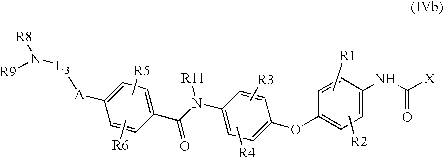

Further advantageously in formula (IVb):
X represents a (C1-C6)alkylamino group, optionally substituted by a (C1-C3)alkoxy(C1-C3)alkyl group, preferably isopropylamino and 1-ethyl-propylamino, or a (C1-C6)dialkylhydrazino group such as defined in formula (I) above, preferably 2,2-dimethylhydrazino, and/or
R11 represents a hydrogen atom, a (C1-C6)alkyl or (C1-C3)alkoxy(C2-C6)alkyl radical, and/or
L3 and R1 to R9 are such as defined in formula (I) above.

Further preferably in formula (IVb):
X represents a (C1-C6)alkylamino group, optionally substituted by a (C1-C3)alkoxy(C1-C3)alkyl group, preferably isopropylamino and 1-ethyl-propylamino, or a (C1-C6)dialkylhydrazino group such as defined in formula (I) above, preferably 2,2-dimethylhydrazino, and/or
R11 represents a hydrogen atom; a (C1-C6)alkyl radical optionally substituted by a heterocycle preferably tetrahydrofurane; a (C1-C3)alkoxy(C2-C6)alkyl radical; a heterocycle such as tetrahydrofurane or tetrahydropyrane, and/or
A is a group of type Ac or Ad, and/or
R7 is a (C1-C6)alkyl radical, and/or
R7, together with R8 and/or R9 and the nitrogen atoms to which they are attached, form a heterocycle such as piperazine or homopiperazine, or
R7, together with Ar3, forms a heterocycle, preferably indoline, and/or
L3 and R1 to R9 are such as defined in formula (I) above.

According to another particular aspect of the invention, a family of preferred compounds corresponds to compounds of formula (I) above, in which L2 represents an amide bond of type L2b such as defined for formula (I) above, and/or A is an oxygen.

One particular sub-family of compounds according to the invention consists of compounds of formula (V) represented below,

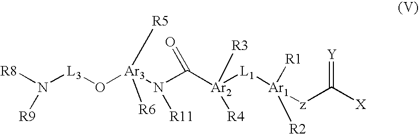

in which Ar1, Ar2 and Ar3 are phenyl radicals, X, Y, Z, L1, L3, R1 to R11 are such as defined in general formula (I) above.

According to preferred variant (Va), L1 is an oxygen and/or Ar1, Ar2 and Ar3 are 4 phenyl radicals. Variant (Va) can preferably be represented as follows:

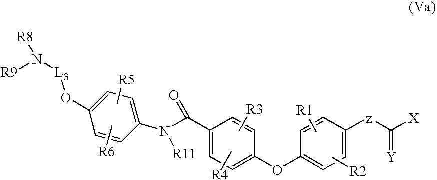

Advantageously, in formula (Va):
X represents a (C1-C6)alkylamino group, optionally substituted by a (C1-C3)alkoxy(C1-C3)alkyl group, preferably isopropylamino and 1-ethyl-propylamino, and/or
Y is an oxygen, and/or
Z is a —NH— radical, and/or
R11 represents a hydrogen atom; a (C1-C6)alkyl radical optionally substituted by a heterocycle preferably tetrahydrofurane; a (C1-C3)alkoxy(C2-C6)alkyl radical; a heterocycle such as tetrahydrofurane or tetrahydropyrane, and/or
L3 is a (C2-C6)alkylene group, and/or
R9, together with L3 and with the nitrogen atom to which it is attached, forms a nitrogen-containing heterocycle, preferably piperidine,
R1 to R6 and R8 are such as defined in formula (I) above.

According to another particular aspect of the invention, a family of preferred compounds corresponds to compounds of formula (I) above, in which L2 represents a simple bond and/or A is an oxygen.

A particular sub-family of compounds according to the invention consists of compounds of formula (VI) represented below,

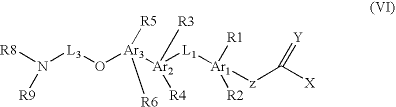

in which Ar1 is a phenyl radical, Ar2 and Ar3 are heteroaryl, aryl or heterocyclic groups such as phenyl, indole, benzofurane, benzoxazole, benzimidazole, 2,3-dihydro-benzofurane, Ar2 and Ar3 not being heteroaryl or heterocyclic groups simultaneously, X, Y, Z, L1, L3 and R1 to R9 are such as defined in general formula (I) above.

For the compounds of family (VI) according to preferred variant (VIa), L1 is an oxygen and Ar1 and Ar3 are 4-phenyl radicals. Variant (VIa) can preferably be represented as follows:

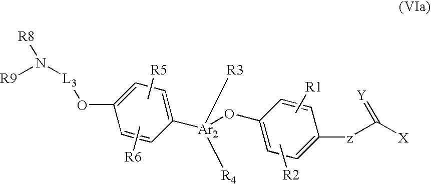

Advantageously in formula (VIa):
X represents a (C1-C6)alkylamino group, optionally substituted by a (C1-C3)alkoxy(C1-C3)alkyl group, preferably isopropylamino and 1-ethyl-propylamino or a (C1-C6)dialkylhydrazino group such as defined in formula (I) above, preferably 2,2-dimethylhydrazino, and/or
Y is an oxygen, and/or
Z is a —NH— radical, and/or
Ar2 is a heteroaryl or heterocyclic group of indole, benzimidazole, benzoxazole, benzofurane type or 2,3-dihydro-benzofurane in which case the group:

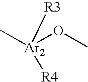

in formula (VIa) above preferably represents:

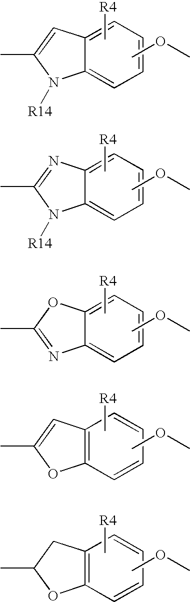

and/or
   L3 is a (C2-C6)alkylene group or a (C3-C8)cycloalkylene group, and/or
   R8 and R9 such as defined in formula (I) above, together and with the nitrogen atom to which they are attached, form a nitrogen-containing heterocycle,
   Or else R9, together with L3 and with the nitrogen atom to which it is attached, forms a nitrogen-containing heterocycle, preferably pyrrolidine, piperidine or homopiperidine,
   R1 to R6 and R14 are such as defined in formula (I) above.

For the compounds of family (VI) according to preferred variant (VIb), L1 is an oxygen and Ar1 and Ar2 are 4-phenyl radicals. Variant (VIb) can preferably be represented as follows:

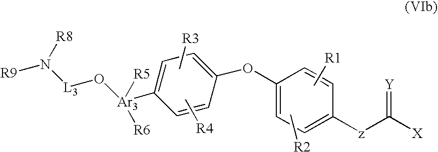

Advantageously, in formula (VIb):
X represents a (C1-C6)alkylamino group, optionally substituted by a (C1-C3)alkoxy(C1-C3)alkyl group, preferably isopropylamino and 1-ethyl-propylamino, or a (C1-C6)dialkylhydrazino group such as defined in formula (I) above, preferably 2,2-dimethylhydrazino, and/or
Y is an oxygen, and/or
Z is a —NH— radical, and/or
Ar3 is a heteroaryl group of benzoxazole, indole, benzimidazole, benzofurane or 2,3-dihydro-benzofurane type, in which case the group:

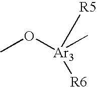

of formula (VIb) above preferably represents:

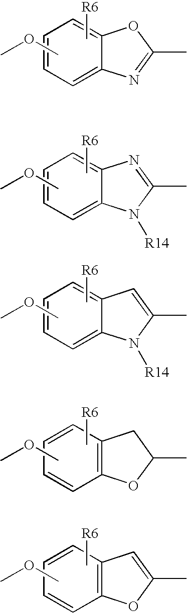

and/or
L3 is a (C2-C6)alkylene group or a (C3-C8)cycloalkylene group, and/or
R8 and R9 such as defined in formula (I) above, together and with the nitrogen atom to which they are attached, form a nitrogen-containing heterocycle,
Or else R9, together with L3 and with the nitrogen atom to which it is attached, forms a nitrogen-containing heterocycle, preferably pyrrolidine, piperidine or homopiperidine,
R1 to R9 and R14 are such as defined in formula (I) above.

As indicated above, the compounds of the invention may be in salt form, in particular acid or base addition salts, preferably compatible with pharmaceutical use.

Among the pharmaceutically acceptable acids, as non-limiting examples mention may be made of hydrochloric, sulfuric, phosphoric, acetic, lactic, tartaric, citric, maleic, methanesulfonic or ethanesulfonic acid. Among pharmaceutically acceptable bases, as non-limiting examples mention may be made of sodium hydroxide, potassium hydroxide, triethylamine and tert-butylamine.

The compounds of the invention may be in the form of different optical isomers, separated or in a mixture, in particular in the form of racemic mixtures. The racemic mixtures can be separated into individual isomers using well-known techniques such as separation of the diastereoisomer salts formed with the optically active acids, followed by reconversion to optically active bases.

The prodrugs of the compounds of formula (I) are also included in the scope of the invention. The prodrugs represent any structure having covalent bonds able to release in vivo a compound meeting general formula (I). Different types of prodrugs are well known in the prior art and described in the literature. Particular mention may be made of the following references: Design of Prodrugs, published by H. Bundgaard, (Elsevier, 1985); Methods in Enzimology, vol 42, p 309-396, published by K. Widder et al (Academic Press, 1985); A Textbook of Drug Design and Development, published by Krosgaard-Larsen and H. Bundgaard, Chapter 5, <<Design and Application of Prodrugs>>, p 113-191 (1991) and H. Bundgaard, Advanced Drug Delivery Reviews, 8, 1-38 (1992).

Specific examples of preferred compounds of the invention are particularly those compounds such as described in examples no 1 to 335 and their pharmaceutically acceptable salts, solvates, hydrates, optical and geometric isomers or their mixtures, more specifically those of examples no 1-3, 5-15, 17-30, 32, 33, 40-58, 62-68, 70, 71, 73, 74, 77-81, 83, 84, 86-120, 123-139, 144-154, 158, 159, 161-167, 170-172, 175-191, 194-236, 238-246 and 250-335 and their pharmaceutically acceptable salts, their solvates, hydrates, optical and geometric isomers or their mixtures, and in particular the compounds described in examples 1, 2, 5, 6, 8, 10, 11, 14, 15, 17-19, 22-27, 40-49, 51-56, 62-66, 68, 70, 71, 86-93, 96-119, 123-137, 144, 150-153, 158, 166, 175-191, 194-205, 209-235, 238-241, 244, 246, 250-273, 275-320 and 322-335 and their pharmaceutically acceptable salts, their solvates, hydrates, optical and geometric isomers or their mixtures.

The particularly preferred compounds of the invention are:
N-5-{4-[3-(1-Ethyl-propyl)-ureido]-2-methoxymethyl-phenoxy}-thiazol-2-yl)-4-(1-isopropyl-piperidin-4-yloxy)-benzamide,
N-(5-{4-[3-(1-Ethyl-propyl)-ureido]-2-methoxy-phenoxy}-thiazol-2-yl)-4-(1-isopropyl-piperidin-4-yloxy)-benzamide,
N-(5-{4-[3-(1-Ethyl-propyl)-ureido]-2-methylcarbamoylmethyl-phenoxy}-thiazol-2-yl)-4-(1-isopropyl-piperidin-4-yloxy)-benzamide,
N-{5-[4-(3-dimethylamino-ureido)-2-methylcarbamoylmethyl-phenoxy]-thiazol-2-yl}-4-(1-isopropyl-piperidin-4-yloxy)-benzamide,
N-(4-{4-[3-(1-Ethyl-propyl)-ureido]-2-methoxymethyl-phenoxy}-phenyl)-4-(1-isopropyl-piperidin-4-yloxy)-benzamide,
N-(4-{4-[3-(1-Ethyl-propyl)-ureido]-2-methoxy-phenoxy}-3-methyl-phenyl)-4-(1-isopropyl-piperidin-4-yloxy)-benzamide,
N-(5-{4-[3-(1-Ethyl-propyl)-urEido]-2-methoxymethyl-phenoxy}-thiazol-2-yl)-4-(1-isopropyl-piperidin-4-yloxy)-N-methyl-benzamide,
N-(4-{4-[3-(1-Ethyl-propyl)-ureido]-phenoxy}-3-methyl-phenyl)-4-(1-isopropyl-piperidin-4-yloxy)-benzamide,
N-(5-{4-[3-(1-Ethyl-propyl)-ureido]-2-methoxymethyl-phenoxy}-thiazol-2-yl)-4-(1-isopropyl-piperidin-3-ylmethoxy)-benzamide,
4-(1-Butyl-piperidin-4-yloxy)-N-(5-{4-[3-(1-ethyl-propyl)-ureido]-2-methoxy-phenoxy}-thiazol-2-yl)-benzamide,
4-(1-Butyl-piperidin-4-yloxy)-N-(5-{4-[3-(1-ethyl-propyl)-ureido]-2-methoxymethyl-phenoxy}-thiazol-2-yl)-benzamide,
4-(1-Butyl-piperidin-4-yloxy)-N-(5-{4-[3-(1-ethyl-propyl)-ureido]-2-fluoro-phenoxy}-thiazol-2-yl)-benzamide,
4-(1-Butyl-piperidin-4-yloxy)-N-(5-{2-ethoxymethyl-4-[3-(1-ethyl-propyl)-ureido]-phenoxy}-thiazol-2-yl)-benzamide,
4-(1-Butyl-piperidin-4-yloxy)-N-(4-{2-ethoxy-4-[3-(1-ethyl-propyl)-ureido]-phenoxy}-phenyl)-benzamide,
4-(1-Butyl-piperidin-4-yloxy)-N-(4-{2-chloro-4-[3-(1-ethyl-propyl)-ureido]-phenoxy}-phenyl)-benzamide,
4-(1-Butyl-piperidin-4-yloxy)-N-{3-[4-(3-dimethylamino-ureido)-2-methoxy-phenoxy]-phenyl}-benzamide,
4-(1-Butyl-piperidin-4-yloxy)-N-(4-{4-[3-(1-ethyl-propyl)-ureido]-2-methoxy-phenoxy}-phenyl)-N-2-hydroxy-ethyl)-benzamide,
4-(1-Butyl-piperidin-4-yloxy)-N-(4-{4-[3-(1-ethyl-propyl)-ureido]-2-methoxy-phenoxy}-phenyl)-N-(3,3,3-trifluoro-propyl)-benzamide,
4-(1-Butyl-piperidin-4-yloxy-N-(4-{4-[3-(1-ethyl-propyl)-ureido]-2-methoxy-phenoxy}-phenyl)-N-(3-methoxy-propyl)-benzamide,
4-(1-Butyl-piperidin-4-yloxy)-N-(4-{4-[3-(1-ethyl-propyl)-ureido]-2-methoxy-phenoxy}-phenyl)-N-(4,4,4-trifluoro-butyl)-benzamide,
4-(1-Butyl-piperidin-4-yloxy)-N-(4-{4-[3-(1-ethyl-propyl)-ureido]-2-isopropyl-phenoxy}-phenyl)-benzamide,
4-(1-Butyl-piperidin-4-yloxy-N-(4-{2-ethoxy-4-[3-(1-ethyl-propyl)-ureido]-phenoxy}-2-fluoro-phenyl)-3-methyl-benzamide,
4-(1-Butyl-piperidin-4-yloxy)-N-(4-{4-[3-(1-ethyl-propyl)-ureido]-2-methoxy-phenoxy}-phenyl)-3-methyl-benzamide,
4-(1-Butyl-piperidin-4-yloxy)-N-(4-{4-[3-(1-ethyl-propyl)-ureido]-2-methoxy-phenoxy}-phenyl)-3-methoxy-benzamide,
4-(1-Butyl-piperidin-4-yloxy-3-chloro-N-(4-{4-[3-(1-ethyl-propyl)-ureido]-2-methoxy-phenoxy}-phenyl)-benzamide,
N-(5-{4-[3-(1-Ethyl-propyl)-ureido]-2-methoxy-phenoxy}-thiazol-2-yl)-4-(1-methyl-piperidin-4-yloxy)-benzamide,
N-(5-{4-[3-(1-Ethyl-propyl)-ureido]-2-methoxymethyl-phenoxy}-thiazol-2-yl)-4-(1-isopropyl-pyrrolidin-3-yloxy)-benzamide,
N-(5-{4-[3-(1-Ethyl-propyl)-ureido]-2-methoxymethyl-phenoxy}-thiazol-2-yl)-3-(1-isopropyl-piperidin-4-yloxy)-benzamide,
N-(4-{3-[3-(1-Ethyl-propyl)-ureido]-phenoxy}-3-methyl-phenyl)-3-(1-isopropyl-piperidin-4-yloxy)-benzamide,
N-(4-{3-[3-(1-Ethyl-propyl)-ureido]-phenoxy}-3-methoxymethyl-phenyl)-3-(1-isopropyl-piperidin-4-yl yloxy)-benzamide,
N-(4-{5-[3-(1-Ethyl-propyl)-ureido]-2-methoxy-phenoxy}-phenyl)-3-(1-isopropyl-piperidin-4-yloxy)-benzamide,
4-(1-Benzyl-piperidin-4-yloxy)-N-(5-{4-[3-(1-ethyl-propyl)-ureido]-2-methoxy-phenoxy}-thiazol-2-yl)-benzamide,
N-(5-{4-[3-(1-Ethyl-propyl)-ureido]-2-methoxymethyl-phenoxy}-thiazol-2-yl)-4-(1-isopropyl-piperidin-3-yloxy)-benzamide,
N-(4-{3-[3-(1-Ethyl-propyl)-ureido]-phenoxy}-3-methyl-phenyl)-4-(1-isopropyl-piperidin-3-yloxy)-benzamide,
N-(4-{5-[3-(1-Ethyl-propyl)-ureido]-2-methoxy-phenoxy}-phenyl)-4-(1-isopropyl-piperidin-3-yloxy)-benzamide,
N-{3-[4-(3-dimethylamino-ureido)-2-methoxy-phenoxy]-phenyl}-4-(1-isopropyl-piperidin-3-yloxy)-benzamide,
N-(5-{4-[3-(1-Ethyl-propyl)-ureido]-2-methoxy-phenoxy}-thiazol-2-yl)-4-(1-isopropyl-piperidin-4-yloxymethyl)-benzamide,
N-(5-{4-[3-(1-Ethyl-propyl)-ureido]-2-methoxymethyl-phenoxy}-thiazol-2-yl)-3-(1-isopropyl-piperidin-3-yloxy)-benzamide,
N-(5-{4-[3-(1-Ethyl-propyl)-ureido]-2-methoxy-phenoxy}-thiazol-2-yl)-1-isopropyl-piperidin-4-ylmethoxy)-benzamide,
N-(4-{2-Ethoxy-4-[3-(1-ethyl-propyl)-ureido]-phenoxy}-phenyl)-4-(8-methyl-8-aza-bicyclo[3.2.1]oct-(3-endo)-yloxy)-benzamide,
N-(4-{4-[3-(1-Ethyl-propyl)-ureido]-2-methoxy-phenoxy}-phenyl)-4-(8-methyl-8-aza-bicyclo[3.2.1]oct-(3-endo)-yloxy)-benzamide,
N-(4-{2-Ethoxy-4-[3-(1-ethyl-propyl)-ureido]-phenoxy}-phenyl)-N-ethyl-4-(8-methyl-8-aza-bicyclo[3.2.1]oct-(3-endo)-yloxy)-benzamide,
N-Ethyl-N-(4-{4-[3-(1-ethyl-propyl)-ureido]-2-methoxy-phenoxy}-phenyl)-4-(8-methyl-8-aza-bicyclo[3.2.1]oct-(3-endo)-yloxy)-benzamide,
N-(4-{4-[3-(1-Ethyl-propyl)-ureido]-2-methoxy-phenoxy}-phenyl)-3-methoxy-4-(8-methyl-8-aza-bicyclo[3.2.1]oct-(3-endo)-yloxy)-benzamide,
3-Chloro-N-(4-{4-[3-(1-ethyl-propyl)-ureido]-2-methoxy-phenoxy}-phenyl)-4-(8-methyl-8-aza-bicyclo[3.2.1]oct-(3-endo)-yloxy)-benzamide,
N-(4-{4-[3-(1-Ethyl-propyl)-ureido]-2-methoxy-phenoxy}-phenyl)-4-(8-methyl-8-aza-bicyclo[3.2.1]octyloxy)-benzamide,
N-(4-{4-[3-(1-Ethyl-propyl)-ureido]-2-methyl-phenoxy}-phenyl)-4-(8-methyl-8-aza-bicyclo[3.2.1]oct-(3-endo)-yloxy)-benzamide,
N-(4-{4-[3-(1-Ethyl-propyl)-ureido]-phenoxy}-2-fluoro-phenyl)-4-(8-methyl-8-aza-bicyclo[3.2.1]oct-(3-endo)-yloxy)-benzamide,
N-(4-{4-[3-(1-Ethyl-propyl)-ureido]-2-methoxy-phenoxy}-phenyl)-2-fluoro-4-(8-methyl-8-aza-bicyclo[3.2.1]oct-(3-endo)-yloxy)-benzamide,
N-(4-{2-Chloro-4-[3-(1-Ethyl-propyl)-ureido]-phenoxy}-phenyl)-N-ethyl-4-(8-methyl-8-aza-bicyclo[3.2.1]oct-(3-endo)-yloxy)-benzamide,
N-(4-{4-[3-(1-Ethyl-propyl)-ureido]-2-methoxy-phenoxy}-phenyl)-4-(2,2,6,6-Tetramethyl-piperidin-4-yloxy)-benzamide,
N-(4-{4-[3-(1-Ethyl-propyl)-ureido]-2-methoxy-phenoxy}-phenyl)-4-(1,2,2,6,6-pentamethyl-piperidin-4-yloxy)-benzamide,
N-(4-{4-[3-(1-Ethyl-propyl)-ureido]-2-methoxy-phenoxy}-phenyl)-4-(2-methyl-2-aza-bicyclo[2.2.2]oct-(5-cis)-yloxy)-benzamide,
N-(5-{4-[3-(1-Ethyl-propyl)-ureido]-2-methoxymethyl-phenoxy}-thiazol-2-yl)-4-(1-isobutyl-1,2,3,6-Tetrahydro-pyridin-4-yl)-benzamide,
N-(5-{4-[3-(1-Ethyl-propyl)-ureido]-2-methoxymethyl-phenoxy}-thiazol-2-yl)-4-(1-propyl-1,2,3,6-Tetrahydro-pyridin-1-yl)-benzamide,
4-(1-Butyl-1,2,3,6-Tetrahydro-pyridin-4-yl)-N-(5-{4-[3-(1-ethyl-propyl)-ureido]-2-methoxymethyl-phenoxy}-thiazol-2-yl)-benzamide,
N-(5-{4-[3-(1-Ethyl-propyl)-ureido]-2-methoxymethyl-phenoxy}-thiazol-2-yl)-4-[1-(3-methyl-butyl-1,2,3,6-Tetrahydro-pyridin-yl]-benzamide,
N-(5-{4-[3-(1-Ethyl-propyl)-ureido]-2-methoxymethyl-phenoxy}-thiazol-2-yl)-4-(1-isopropyl-piperidin-yl)-benzamide,
3-(4-Hydroxy-piperidin-1-ylmethyl)-1-isopropyl-1H-indolecarboxylic acid (5-{4-[3-(1-ethyl-propyl)-ureido]-2-methoxymethyl-phenoxy}-thiazol-2-yl)-amide,
2-[2-(4-Hydroxy-piperidin-1-yl)-ethyl]-benzofuran-6-carboxylic acid (5-{4-[3-(1-ethyl-propyl)-ureido]-2-methoxymethyl-phenoxy}-thiazol-2-yl)amide,
N-(5-{4-[3-(1-Ethyl-propyl)-ureido]-2-methoxy-phenoxy}-thiazol-2-yl)-4-(3-piperidin-1-yl-propoxy)-benzamide,
N-(4-{4-[3-(1-Ethyl-propyl)-ureido]-2-methoxy-phenoxy}-phenyl)-4-(1-isopropyl-piperidin-4-yloxy)-benzamide,
4-(1-Butyl-piperidin-4-yloxy)-N-(4-{4-[3-(1-ethyl-propyl)-ureido]-2-methyl-phenoxy}-phenyl)-benzamide,
N-(4-{4-[3-(1-Ethyl-propyl)-ureido]-phenoxy}-3-methoxymethyl-phenyl)-4-(1-isopropyl-piperidin-4-yloxy)-benzamide,
N-(4-{4-[3-(1-Ethyl-propyl)-ureido]-phenoxy}-3-methoxy-phenyl)-4-(1-isopropyl-piperidin-4-yloxy)-benzamide,
N-(5-{4-[3-(1-Ethyl-propyl)-ureido]-2-methylcarbamoylmethyl-phenoxy}-thiazol-2-yl)-4-(1-isobutyl-1,2,3,6-Tetrahydro-pyridin-4-yl)-benzamide,
N-(4-{5-[3-(1-Ethyl-propyl)-ureido]-2-methoxy-phenoxy}-phenyl)-4-(1-isopropyl-piperidin-4-yloxy)-benzamide,
{(4-cis)-[4-(4-{4-[3-(1-Ethyl-propyl)-ureido]-2-methoxy-phenoxy}-phenylcarbamoyl)-phenoxy]-cyclohexyl}-trimethyl-ammonium,
N-(4-{4-[3-(1-Ethyl-propyl)-ureido]-phenoxy}-3-methyl-phenyl)-4-(3-piperidin-1-yl-propoxy)-benzamide,
N-(5-{4-[3-(1-Ethyl-propyl)-ureido]-2-methoxymethyl-phenoxy}-thiazol-2-yl)-4-(1-isopropyl-piperidin-4-ylmethyl)-benzamide,
N-(5-{4-[3-(1-Ethyl-propyl)-ureido]-2-methoxymethyl-phenoxy}-thiazol-2-yl)-4-(1-isopropyl-piperidin-4-ylidenemethyl)-benzamide,
4-[1-(2-Dimethylamino-Acetyl)-1,2,3,6-Tetrahydro-pyridin-4-yl]-N-(5-{4-[3-(1-ethyl-propyl)-ureido]-2-methoxymethyl-phenoxy}-thiazol-2-yl)-benzamide,
1-Isopropyl-2-(2-piperidin-1-yl-ethyl)-1H-benzoimidazole-5-carboxylic acid (5-{4-[3-(1-ethyl-propyl)-ureido]-2-methoxy-phenoxy}-thiazol-2-yl)-amide,
1-Isopropyl-2-(2-piperidin-1-yl-ethyl)-1H-benzoimidazole-5-carboxylic acid (4-{4-[3-(1-ethyl-propyl)-ureido]-phenoxy}-3-methyl-phenyl)-amide,
1-[3-(4-Hydroxy-piperidin-1-yl)propyl]-1H-indole-5-carboxylic acid (5-{4-[3-(1-ethyl-propyl)-ureido]-2-methoxy-phenoxy}-thiazol-2-yl)-amide,
1-(2-Piperidin-1-yl-ethyl)-1H-indole-5-carboxylic acid (5-{4-[3-(1-ethyl-propyl)-ureido]-2-methoxy-phenoxy}-thiazol-2-yl)-amide,
4-[1,4′]Bipiperidin-1′-yl-N-(5-{4-[3-(1-ethyl-propyl)-ureido]-2-methoxy-phenoxy}-thiazol-2-yl)-benzamide,
4-[Ethyl-(3-piperidin-1-yl-propionyl)-amino]-N-(5-{4-[3-(1-ethyl-propyl)-ureido]-2-methoxy-phenoxy}-thiazol-2-yl)-benzamide,
4-[Acetyl-(2-piperidin-1-yl-ethyl)amino]-N-(5-{4-[3-(1-ethyl-propyl)-ureido]-2-methoxy-phenoxy}-thiazol-2-yl)-benzamide,
4-[Ethyl-(3-piperidin-1-yl-propyl)-amino]-N-(5-{4-[3-(1-ethyl-propyl)-ureido]-2-methoxy-phenoxy}-thiazol-2-yl)-benzamide,
N-5-{4-[3-(1-Ethyl-propyl)-ureido]-2-methoxy-phenoxy}-thiazol-2-yl)-4-(3-piperidin-1-yl-propionylamino)-benzamide,
4-[Ethyl-(3-piperidin-1-yl-propionyl)-amino]-N-(5-{4-[3-(1-ethyl-propyl)-ureido]-phenoxy}-thiazol-2-yl)-benzamide,
4-[Acetyl-(2-piperidin-1-yl-ethyl)amino]-N-(4-{4-[3-(1-ethyl-propyl)-ureido]-phenoxy}-3-methyl-phenyl)-benzamide,
4-(4-Ethyl-piperazine-1-carbonyl)-N-(4-{4-[3-(1-ethyl-propyl)-ureido]-phenoxy}-3-methyl-phenyl)-benzamide,
5-(3-Isopropyl-ureido)-2-(4-{[1-(2-piperidin-1-yl-ethyl-1H-indole-5-carbonyl]-amino}-phenoxy)-methyl benzoate,
4-(1-Butyl-piperidin-4-yloxy-N-(4-{2-ethoxy-4-[3-(1-ethyl-propyl)-ureido]-phenoxy}-phenyl)-3-methoxy-benzamide,
4-(1-Butyl-piperidin-4-yloxy)-N-(4-{4-[3-(1-ethyl-propyl)-ureido]-phenoxy}-2-fluoro-phenyl)-3-methyl-benzamide,
N-(4-{4-[3-(1-ethyl-propyl)-ureido]-2-methoxymethyl-phenoxy}-3-methyl-phenyl)-4-(1-isopropyl-piperidin-4-yloxy)-benzamide,
4-(1-Butyl-piperidin-4-yloxy)-N-ethyl-N-(4-{4-[3-(1-ethyl-propyl)-ureido]-2-methoxy-phenoxy}-phenyl)-benzamide,
N-(4-{4-[3-(1-Ethyl-propyl)-ureido]-2-methoxy-phenoxy}-phenyl)-4-(1-methyl-piperidin-4-yloxy)-benzamide,
4-(1-Butyl-piperidin-4-yloxy)-N-ethyl-N-(4-{4-[3-(1-ethyl-propyl)-ureido]-2-methoxy-phenoxy}-phenyl)-3-methoxy-benzamide,
4-(1-Butyl-piperidin-4-yloxy)-N-ethyl-N-(4-{4-[3-(1-ethyl-propyl)-ureido]-2-methoxy-phenoxy}-phenyl)-3-methyl-benzamide,
N-(5-{4-[3-(1-Ethyl-propyl)-ureido]-2-methoxymethyl-phenoxy}-thiazol-2-yl)-4-(1-isopropyl-1,2,3,6-Tetrahydro-pyridin-4-yl)-benzamide,
1-(3-Piperidin-1-yl-propyl)-1H-indole-5-carboxylic acid (4-{4-[3-(1-ethyl-propyl)-ureido]-2-methoxy-phenoxy}-3-methyl-phenyl)-amide,
1-(2-Piperidin-1-yl-ethyl)-1H-indole-5-carboxylic acid (4-{4-[3-(1-ethyl-propyl)-ureido]-phenoxy}-3-methyl-phenyl)-amide,
4-(1-Butyl-piperidin-4-yloxy)-N-(4-{4-[3-(1-ethyl-propyl)-ureido]-2-methoxy-phenoxy}-phenyl)-N-(2,2,2-trifluoro-ethyl)-benzamide,
N-(4-{4-[3-(1-Ethyl-propyl)-ureido]-2-methoxy-phenoxy}-phenyl)-4-(8-methyl-8-aza-bicyclo[3.2.1]oct-(3-endo-yloxy)-N-(2,2,2-trifluoroethyl)-benzamide,
4-(1-Butyl-piperidin-4-yloxy)-N-(4-{4-[3-(1-ethyl-propyl)-ureido]-2-methoxy-phenoxy}-phenyl)-3,5-dimethyl-benzamide,
N-(4-{4-[3-(1-Ethyl-propyl)-ureido]-2-methoxy-phenoxy}-phenyl)-[1,(cis,cis-2,6)-trimethyl-piperidin-cis-4)-yloxy]-benzamide,
2-Chloro-N-(4-{4-[3-(1-ethyl-propyl)-ureido]-2-methoxy-phenoxy}-phenyl)-4-(8-methyl-8-aza-bicyclo[3.2.1]oct-(3-endo)-yloxy)-benzamide,
N-(4-{4-[3-(1-Ethyl-propyl)-ureido]-2-methoxy-phenoxy}-phenyl)[1,(cis,cis-2,6)-trimethyl-piperidin-trans-4)-yloxy]-benzamide,
4-(1-Butyl-piperidin-4-yloxy)-3-chloro-N-ethyl-N-(4-{4-[3-(1-ethyl-propyl)-ureido]-2-methoxy-phenoxy}-phenyl)-benzamide,
4-(1-Butyl-piperidin-4-yloxy-N-(4-{4-[3-(1-ethyl-propyl)-ureido]-2-methoxy-phenoxy}-phenyl)-benzamide,
4-(1-Butyl-piperidin-4-yloxy)-N-(4-{4-[3-(1-ethyl-propyl)-ureido]-2-propoxy-phenoxy}-phenyl)-3-methyl-benzamide,
4-(1-Butyl-piperidin-4-yloxy)-N-(4-{4-[3-(1-ethyl-propyl)-ureido]-2-methoxy-phenoxy}-phenyl)-2-fluoro-benzamide,
4-(1-Butyl-piperidin-4-yloxy)-N-(4-{4-[3-(1-ethyl-propyl)-ureido]-phenoxy}-phenyl)-benzamide,
1-(4-{1-[4-(1-Butyl-piperidin-4-yloxy)-benzoyl]-2,3-dihydro-1H-indol-5-yloxy}-3-methoxy-phenyl)-3-(1-ethyl-propyl)-urea,
4-(1-Butyl-piperidin-4-yloxy)-N-(4-{4-[3-(1-ethyl-propyl)-ureido]-2-methoxymethyl-phenoxy}-3-methyl-phenyl)-benzamide,
N-(4-{4-[3-(1-Ethyl-propyl)-ureido]-phenoxy}-phenyl)-4-(1-isopropyl-piperidin-4-yloxy)-benzamide,
4-(1-Butyl-piperidin-4-yloxy)-N-(4-{4-[3-(1-ethyl-propyl)-ureido]-2-fluoro-phenoxy}-phenyl)-benzamide,
1-(1-Ethyl-propyl)-3-(3-methoxyl-4-{1-[4-(8-methyl-8-aza-bicyclo[3.2.1]oct-(3-endo)-yloxy)-benzoyl]-2,3-dihydro-1H-indol-5-yloxy}-phenyl)-urea,
N-(4-{4-[3-(1-Ethyl-propyl)-ureido]-2-methoxy-phenoxy}-phenyl)-4-(8-methyl-8-aza-bicyclo[3.2.1]oct-(3-exo)-yloxy)-benzamide,
N-(4-{2-Ethyl-4-[3-(1-ethyl-propyl)-ureido]-phenoxy}-phenyl)-4-(8-methyl-8-aza-bicyclo[3.2.1]oct-(3-endo)-yloxy)-benzamide,
4-(1-Butyl-piperidin-4-yloxy)-N-{4-[4-(3-isopropyl-ureido)-phenoxy]-2-methoxy-phenyl}-benzamide,
1-(4-{1-[4-(1-Butyl-piperidin-4-yloxy)-3-methyl-benzoyl]-2,3-dihydro-1H-indol-5-yloxy}-3-methoxy-phenyl)-3-(1-ethyl-propyl)-urea,
4-(1-Butyl-piperidin-4-yloxy)-N-(4-{2-ethyl-4-[3-(1-ethyl-propyl)-ureido]-phenoxy}-phenyl)-benzamide,
N-(4-{4-[3-(1-Ethyl-propyl)-ureido]-2-methoxy-phenoxy}-phenyl)-4-(1-isopropyl-piperidin-4-ylmethoxy)-benzamide,
N-(4-{4-[3-(1-Ethyl-propyl)-ureido]-2-trifluoromethyl-phenoxy}-phenyl)-4-(8-methyl-8-aza-bicyclo[3.2.1]oct-(3-endo)-yloxy)-benzamide,
4-(1-Butyl-piperidin-4-yloxy)-N-{4-[4-(3-dimethylamino-ureido)-phenoxy]-3-methoxymethyl-phenyl}-3-methyl-benzamide,
4-(1-Butyl-piperidin-4-yloxy)-N-(4-{4-[3N,N-dimethyl-amino)ureido]-phenoxy}-3-methoxymethyl-phenyl)-3-methoxy-benzamide,
4-(1-Butyl-piperidin-4-yloxy)-N-(4-{4-[3-(1-ethyl-propyl)-ureido]-phenoxy}-3-trifluoromethyl-phenyl)-benzamide,
4-(1-Butyl-piperidin-4-yloxy)-N-{-[4-(3-isopropyl-ureido-benzyl]-phenyl}-benzamide,
N-(4-{4-[3-(1-Ethyl-propyl)-ureido]-phenoxy}-phenyl)-4-(8-methyl-8-aza-bicyclo [3.2.1]oct-(3-endo)-yloxy)-benzamide,
N-(4-{2-Chloro-4-[3-(1-ethyl-propyl)-ureido]-phenoxy}-phenyl)-4-(8-methyl-8-aza-bicyclo[3.2.1]oct-3-endo)-yloxy)-benzamide,
N-(4-{4-[3-(1-Ethyl-propyl)-ureido]-2-methoxymethyl-phenoxy}-phenyl)-4-(8-methyl-8-aza-bicyclo[3.2.1]oct-(3-endo)-yloxy)-benzamide,
N-(4-{4-[3-(1-Ethyl-propyl)-ureido]-2-fluoro-phenoxy}-phenyl)-4-(8-methyl-8-aza-bicyclo[3.2.1]oct-(3-endo)-yloxy)-benzamide,
4-(1-Butyl-piperidin-4-yloxy)-N-(4-{2-chloro-4-[3-(1-ethyl-propyl)-ureido]-phenoxy}-2-fluoro-phenyl)-benzamide,
4-(1-Butyl-piperidin-4-yloxy)-N-(4-{2-ethoxy-4-[3-(1-ethyl-propyl)-ureido]-phenoxy}-phenyl)-3-methyl-benzamide,
N-(4-{4-[3-(1-Ethyl-propyl)-ureido]-3-fluoro-phenoxy}-phenyl)-4-(8-methyl-8-aza-bicyclo[3.2.1]oct-(3-endo)-yloxy)-benzamide,
4-(1-Butyl-piperidin-4-yloxy)-N-(4-{4-[3-(1-ethyl-propyl)-ureido]-2-methoxy-phenoxy}-phenyl)-3-fluoro-benzamide,
4-(1-Butyl-piperidin-4-yloxy)-N-(4-{4-[3-(1-ethyl-propyl)-ureido]-3-fluoro-phenoxy}-phenyl)-benzamide,
4-(1-Butyl-piperidin-4-yloxy)-N-{4-[4-(3-isopropyl-ureido-2-methoxy-phenoxy]-phenyl}-benzamide,
4-(1-Butyl-piperidin-4-yloxy)-N-(4-{4-[3-(1-ethyl-propyl)-ureido]-phenoxy}-2-fluoro-phenyl)-benzamide,
4-(1-Butyl-piperidin-4-yloxy)-N-(4-{4-[3-(1-ethyl-propyl)-ureido]-2-methoxy-phenoxy}-phenyl)-3-trifluoromethyl-benzamide,
4-(1-Butyl-piperidin-4-yloxy)-N-(4-{4-[3-1 ethyl-propyl)-ureido]-phenoxy}-2-methoxy-phenyl)-3-methyl-benzamide,
4-(1-Butyl-piperidin-4-yloxy)-N-(4-{4-[3-(1-ethyl-propyl)-ureido]-phenoxy}-phenyl)-3-methyl-benzamide,
N-(4-{4-[3-(1-Ethyl-propyl)-ureido]-phenoxy}-3-methyl-phenyl)-4-(8-methyl-8-aza-bicyclo[3.2.1]oct-(3-endo)-yloxy)-benzamide,
4-(1-Butyl-piperidin-4-yloxy)-N-{4-[4-(3-isopropyl-ureido)-phenoxy]-3,5-dimethyl-phenyl}-benzamide,
N-(4-{3-[3-(1-Ethyl-propyl)-ureido]-phenoxy}-3-methyl-phenyl)-4-(1-isopropyl-piperidin-4-yloxy)-benzamide,
4-(1-Butyl-piperidin-4-yloxy)-N-{4-[4-(3-isopropyl-ureido)-phenoxy]-2,5-dimethyl-phenyl}-benzamide,
4-(1-Butyl-piperidin-4-yloxy)-N-{4-[4-(3-isopropyl-ureido)-3-methoxy-phenoxy]-phenyl}-benzamide,
4-(1-Isopropyl-1,2,3,6-Tetrahydro-pyridin-4-yl)-N-{4-[4-(3-isopropyl-ureido)-2-methoxy-phenoxy]-phenyl}-benzamide,
(1-Ethyl-propyl)-carbamate of 4-{4-[4-(1-isopropyl-piperidin-4-yloxy)-benzoylamino]-2-methyl-phenoxy}-phenyl,
N-(4-{4-[3-(1-Ethyl-propyl)-ureido]-2-methoxy-phenoxy}-phenyl)-4-(1-methyl-1,2,3,6-Tetrahydro-pyridin-4-yl)-benzamide,
N-(4-{4-[3-(1-Ethyl-propyl)-ureido]-2-methoxy-phenoxy}-3-methyl-phenyl)-4-(1-ethyl-1,2,3,6-Tetrahydro-pyridin-4-yl)-benzamide,
4-(1-Butyl-1,2,3,6-Tetrahydro-pyridin-4-yl)-N-(4-{4-[3-(1-ethyl-propyl)-ureido]-2-methoxy-phenoxy}-3-methyl-phenyl)-benzamide,
4-(1-Isobutyl-1,2,3,6-Tetrahydro-pyridin-4-yl)-N-{4-[4-(3-isopropyl-ureido)-2-methoxy-phenoxy]-phenyl}-benzamide,
1-(2-Piperidin-1-yl-ethyl)-1H-indole-5-carboxylic acid (4-{4-[3-(1-ethyl-propyl)-ureido]-2-methylcarbamoyl-phenoxy}-phenyl)-amide,
4-[Acetyl(3-piperidin-1-yl-propyl-amino]-N-(4-{4-[3-(1-ethyl-propyl)-ureido]-phenoxy}-3-methyl-phenyl)-benzamide,
N-Ethyl-N-(4-{4-[3-(1-ethyl-propyl)-ureido]-2-methoxy-phenoxy}-phenyl)-3-fluoro-4-(8-methyl-8-aza-bicyclo[3.2.1]oct-(3-endo)-yloxy)-benzamide,
N-(4-{4-[3-(1-Ethyl-propyl)-ureido]-2-methoxy-phenoxy}-phenyl)-3-fluoro-4-(8-methyl-8-aza-bicyclo[3.2.1]oct-(3-endo)-yloxy)-benzamide,
N-(4-{4-[3-(1-Ethyl-propyl)-ureido]-2-methoxy-phenoxy}-3-methyl-phenyl)-4-(1-methyl-1,2,3,6-Tetrahydro-pyridin-4-yl)-benzamide,
4-(1-Butyl-piperidin-4-yloxy)-N-{4-[3-isopropyl-ureido-phenoxy]-phenyl}-benzamide,
4-[1-(2-Dimethylamino-Acetyl)-piperidin-4-yloxy]-N-(4-{4-[3-(1-ethyl-propyl)-ureido]-2-methoxy-phenoxy}-phenyl)-benzamide,
4-(1-Butyl-piperidin-4-yloxy)-N-{4-[4-(2-dimethylamino-Acetylamino)-2-methoxy-phenoxy]-phenyl}-benzamide,
4-(1-Butyl-piperidin-4-yloxy)-N-{4-[3-hydroxymethyl-4-(3-isopropyl-ureido)-phenoxy]-phenyl}-benzamide,
5-[3-(1-Ethyl-propyl)-ureido]-N-methyl-2-{4-[4-(3-piperidin-1-yl-propoxy)-benzoylamino]-phenoxy}-benzamide,
4-[(4-cis)-Dimethylamino-cyclohexyloxy]-N-(4-{4-[3-(1-ethyl-propyl)-ureido]-2-methoxy-phenoxy}-phenyl)-benzamide,
5-[3-(1-Ethyl-propyl)-ureido]-2-(4-{4-[3-(4-hydroxy-piperidin-1-yl)-propoxy]-benzoylamino}-phenoxy)-N-methyl-benzamide,
4-(1-Butyl-piperidin-4-yloxy)-N-{4-[4-(3-isopropyl-ureido)-phenylsulfanyl]-phenyl}-benzamide,
N-(4-{4-[3-(1-Ethyl-propyl)-ureido]-3-fluoro-phenoxy}-phenyl)-4-(1-methyl-1,2,3,6-Tetrahydro-pyridin-4-yl)-benzamide,
N-(4-{4-[3-(1-Ethyl-propyl)-ureido]-2-methoxymethyl-phenoxy}-phenyl)-4-(1-isopropyl-1,2,3,6-Tetrahydro-pyridin-4-yl)-benzamide,
N-(4-{4-[3-(1-Ethyl-propyl)-ureido]-2-methoxy-phenoxy}-3-methyl-phenyl)-4-(1-isopropyl-1,2,3,6-Tetrahydro-pyridin-4-yl)-benzamide,
N-(4-{4-[3-(1-Ethyl-propyl)-ureido]-2-methoxy-phenoxy}-phenyl)-4-(1-isopropyl-1,2,3,6-Tetrahydro-pyridin-4-yl)-benzamide,
N-(4-{4-[3-(1-Ethyl-propyl)-ureido]-2-methoxy-phenoxy}-3-methyl-phenyl)-4-(1-propyl-1,2,3,6-Tetrahydro-pyridin-4-yl)-benzamide,
1-[3-(4-Hydroxy-piperidin-1-yl)propyl]-1H-indole-5-carboxylic acid (4-{4-[3-(1-ethyl-propyl)-ureido]-2-methylcarbamoyl-phenoxy}-phenyl)-amide,
1-(3-Piperidin-1-yl-propyl)-1H-indole-5-carboxylic acid (4-{4-[3-(1-ethyl-propyl)-ureido]-phenoxy}-3-methyl-phenyl)-amide,
3-Methyl-1-(2-piperidin-1-yl-ethyl)-1H-indole-5-carboxylic acid (4-{4-[3-(1-ethyl-propyl)-ureido]-phenoxy}-3-methyl-phenyl)-amide,
3-Acetyl-1-(2-piperidin-1-yl-ethyl)-1H-indole-5-carboxylic acid (4-{4-[3-(1-ethyl-propyl)-ureido]-phenoxy}-3-methyl-phenyl)-amide,
4-[Acetyl-(3-piperidin-1-yl-propyl)-amino]-N-(5-{4-[3-(1-ethyl-propyl)-ureido]-2-methoxy-phenoxy}-thiazol-2-yl)-benzamide,
2-(4-{4-[Acetyl-(3-diethylamino-propyl)-amino]-benzoylamino}-phenoxy)-5-[3-(1-ethyl-propyl)-ureido]-N-methyl-benzamide,
4-[Ethyl-(3-piperidin-1-yl-propionyl)-amino]-N-(4-{4-[3-(1-ethyl-propyl)-ureido]-phenoxy}-3-methyl-phenyl)-benzamide,
4-[Ethyl-(3-piperidin-1-yl-propyl)-amino]-N-(4-{4-[3-(1-ethyl-propyl)-ureido]-phenoxy}-3-methyl-phenyl)-benzamide,
N-(4-{4-[3-(1-Ethyl-propyl)-ureido]-phenoxy}-3-methyl-phenyl)-4-(3-piperidin-1-yl-propionylamino)-benzamide,
1-(3-Piperidin-1-yl-propyl)-1H-indole-5-carboxylic acid (4-{4-[3-(1-ethyl-propyl)-ureido]-2-methylcarbamoyl-phenoxy}-phenyl)-amide,
4-(1-Benzyl-piperidin-4-yloxy)-N-(4-{4-[3-(1-ethyl-propyl)-ureido]-2-methoxy-phenoxy}-phenyl)-benzamide,
N-(4-{4-[3-(1-Ethyl-propyl)-ureido]-2-methoxy-phenoxy}-phenyl)-4-(piperidin-4-yloxy)-benzamide,
N-(4-{4-[3-(1-Ethyl-propyl)-ureido]-phenoxy}-3-methyl-phenyl-4-(piperidin-4-yloxy)-benzamide,
N-(4-{4-[3-(1-Ethyl-propyl)-ureido]-2-methoxy-phenoxy}-phenyl)-4-(1-propyl-piperidin-4-yloxy)-benzamide,
4-{4-[4-(4-{4-[3-(1-ethyl-propyl)-ureido]-2-methoxy-phenoxy}-phenylcarbamoyl)-phenoxy]-piperidin-1-yl}-butyl acetate,
4-[1-(3-Dimethylamino-propyl)-piperidin-4-yloxy]-N-(4-{4-[3-(1-ethyl-propyl)-ureido]-2-methoxy-phenoxy}-phenyl)-N-(2-methoxy-ethyl)-benzamide,
4-[1-(3-Dimethylamino-propyl)-piperidin-4-yloxy]-N-(4-{4-[3-(1-ethyl-propyl)-ureido]-2-methoxy-phenoxy}-phenyl)-N-isobutyl-benzamide,
4-(1-Butyl-piperidin-4-yloxy)-N-(4-{4-[3-(1-ethyl-propyl)-ureido]-2-methoxy-phenoxy}-3-methyl-phenyl)-benzamide,
4-(8-Butyl-8-aza-bicyclo[3.2.1]oct-(3-endo)-yloxy)-N-(4-{4-[3-(1-ethyl-propyl)-ureido]-2-methoxy-phenoxy}-phenyl)-benzamide,
4-(8-Ethyl-8-aza-bicyclo[3.2.1]oct-(3-endo)-yloxy-N-(4-{4-[3-(1-ethyl-propyl)-ureido]-2-methoxy-phenoxy}-phenyl)-benzamide,
N-(4-{4-[3-(1-Ethyl-propyl)-ureido]-2-methoxy-phenoxy}-phenyl)-4-[8-(3-methoxy-propyl)-8-aza-bicyclo[3.2.1]oct-(3-endo)-yloxy]-benzamide,
N-(4-{4-[3-(1-Ethyl-propyl)-ureido]-2-methoxy-phenoxy}-phenyl)-[8-(2-methoxy-ethyl)-8-aza-bicyclo[3.2.1]oct-(3-endo)-yloxy]-benzamide,
N-(4-{4-[3-(1-Ethyl-propyl)-ureido]-2-methoxy-phenoxy}-phenyl)-4-[1-(4,4,4-trifluoro-butyl)-piperidin-4-yloxy]-benzamide,
4-[1-(2-Diethylamino-ethyl)-piperidin-4-yloxy]-N-(4-{4-[3-(1-ethyl-propyl)-ureido]-2-methoxy-phenoxy}-3-methyl-phenyl)-benzamide,
N-(4-{4-[3-(1-Ethyl-propyl)-ureido]-2-methoxy-phenoxy}-phenyl)-4-[1-(4-fluoro-butyl)-piperidin-4-yloxy]-benzamide,
4-[1-(1-Ethyl-propyl)-piperidin-4-yloxy]-N-(4-{4-[3-(1-ethyl-propyl)-ureido]-phenoxy}-3-methyl-phenyl)-benzamide,
{4-[4-(4-{4-[3-(1-Ethyl-propyl)-ureido]-phenoxy}-3-methyl-phenylcarbamoyl)-phenoxy]-piperidin-1-yl}-ethyl acetate,
{4-[4-(4-{4-[3-(1-Ethyl-propyl)-ureido]-phenoxy}-3-methyl-phenylcarbamoyl)-phenoxy]-piperidin-1-yl}-acetic acid,
N-(4-{4-[3-(1-Ethyl-propyl)-ureido]-2-methoxy-phenoxy}-phenyl)-4-[1-(4-hydroxy-butyl)-piperidin-4-yloxy]-benzamide,
4-{(3-endo)-[4-(4-{4-[3-(1-ethyl-propyl)-ureido]-2-methoxy-phenoxy}-phenylcarbamoyl)-phenoxy]-8-aza-bicyclo[3.2.1]oct-8-yl}-butyl acetate,
4-[8-(3-Dimethylamino-propyl)-8-aza-bicyclo[3.2.1]oct-(3-endo)-yloxy]-N-(4-{4-[3-(1-ethyl-propyl)-ureido]-2-methoxy-phenoxy}-phenyl)-benzamide,
4-[1-(3-Dimethyl-propyl)-piperidin-4-yloxy]-N-(4-{4-[3-(1-ethyl-propyl)-ureido]-2-methoxy-phenoxy}-phenyl)-benzamide,
N-(4-{4-[3-(1-Ethyl-propyl)-ureido]-2-methoxy-phenoxy}-phenyl)-4-(8-propyl-8-aza-bicyclo[3.2.1]oct-3-yloxy)-benzamide,
4-(1-sec-Butyl-piperidin-4-yloxy)-N-(4-{4-[3-(1-ethyl-propyl)-ureido]-2-methoxy-phenoxy}-phenyl)-benzamide,
4-(1-Butyl-piperidin-4-yloxy)-N-(4-{4-[3-(1-ethyl-propyl)-ureido]-2-methoxy-phenoxy}-phenyl)-N-(2-methoxy-ethyl)-benzamide,
N-(4-{4-[3-(1-Ethyl-propyl)-ureido]-2-methoxy-phenoxy}-phenyl)-4-[8-(2-hydroxy-ethyl)-8-aza-bicyclo[3.2.1]oct-(3-endo)-yloxy]-benzamide,
N-(4-{4-[3-(1-Ethyl-propyl)-ureido]-2-methoxy-phenoxy}-phenyl)-4-[8-(4,4,4-trifluoro-butyl)-8-aza-bicyclo[3.2.1]oct-(3-endo)-yloxy]-benzamide,
N-(4-{4-[3-(1-Ethyl-propyl)-ureido]-2-methoxy-phenoxy)}-phenyl)-4-[8-(3-hydroxy-propyl)-8-aza-bicyclo[3.2.1]oct-(3-endo)-yloxy]-benzamide,
N-(4-{4-[3-(1-Ethyl-propyl)-ureido]-2-methoxy-phenoxy}-phenyl)-4-(8-isopropyl-8-aza-bicyclo[3.2.1]oct-(3-endo)-yloxy)-benzamide,
4-(1-Cyclohexylmethyl-piperidin-4-yloxy)-N-(4-{4-[3-(1-ethyl-propyl)-ureido]-phenoxy}-3-methyl-phenyl)-benzamide,
4-[1-(2-Ethyl-butyl)-piperidin-4-yloxy]-N-(4-{4-[3-(1-(ethyl-propyl)-ureido]-phenoxy}-3-methyl-phenyl)-benzamide,
N-(4-{4-[3-(1-Ethyl-propyl)-ureido]-phenoxy}-3-methyl-phenyl)-[1-(2-methoxy-ethyl)-piperidin-4-yloxy]-benzamide,
N-(4-{4-[3-(1-Ethyl-propyl)-ureido]-phenoxy}-3-methyl-phenyl)[1-(2-hydroxy-ethyl)-piperidin-4-yloxy]-benzamide,
N-(4-{4-[3-(1-Ethyl-propyl)-ureido]-2-methoxy-phenoxy}-phenyl)-4-[8-(4-hydroxy-butyl)-8-aza-bicyclo[3.2.1]oct-(3-endo)-yloxy]-benzamide,
4-(8-Aza-bicyclo[3.2.1]oct-(3-endo)-yloxy)-N-(4-{4-[3-(1-ethyl-propyl)-ureido]-2-methoxy-phenoxy}-phenyl)-benzamide,
4-(8-Aza-bicyclo[3.2.1]oct-(3-endo)-yloxy)-N-(4-{4-[3-(1-ethyl-propyl)-ureido]-2-methoxy-phenoxy}-phenyl)-N-(2-methoxy-ethyl)-benzamide,
N-(4-{4-[3-(1-Ethyl-propyl)-ureido]-2-methoxy-phenoxy}-phenyl)-4-[1-(3-methoxy-propyl)-piperidin-4-yloxy]-benzamide,
N-(4-{4-[3-(1-Ethyl-propyl)-ureido]-2-methoxy-phenoxy}-phenyl)[1¶-hydroxy-ethyl)-piperidin-4-yloxy]-benzamide,
N-(4-{4-[3-(1-Ethyl-propyl)-ureido]-2-methoxy-phenoxy}-3-methyl-phenyl)-4-[1-(3-hydroxy-propyl)-piperidin-4-yloxy]-benzamide,
4-[1-(2-Ethoxy-ethyl)piperidin-4-yloxy]-N-(4-{4-[3-(1-ethyl-propyl)-ureido]-2-methoxy-phenoxy}-phenyl)-benzamide,
N-(4-{4-[3-(1-Ethyl-propyl)-ureido]-2-methoxy-phenoxy}-phenyl)-4-[1-(2-methoxy-ethyl)-piperidin-4-yloxy]-benzamide,
N-(4-{4-[3-(1-Ethyl-propyl)-ureido]-2-methoxy-phenoxy}-3-methyl-phenyl)-4-[1-(3-methyl-butyl)-piperidin-4-yloxy]-benzamide,
4-(1-Butyl-piperidin-4-yloxy)-N-(4-{4-[3-(1-ethyl-propyl)-ureido]-2-methoxy-phenoxy}-phenyl)-N-isobutyl-benzamide,
4-(1-sec-Butyl-piperidin-4-yloxy)-N-(4-{4-[3-(1-ethyl-propyl)-ureido]-phenoxy}-3-methyl-phenyl)-benzamide,
N-(4-{4-[3-(1-Ethyl-propyl)-ureido]-2-methoxy-phenoxy}-phenyl)-4-[1-(3,3,3-trifluoro-propyl)-piperidin-4-yloxy]-benzamide,
N-(4-{4-[3-(1-Ethyl-propyl)-ureido]-2-methoxy-phenoxy}-phenyl)-4-[1-(3-hydroxy-propyl)-piperidin-4-yloxy]-benzamide,
N-(4-{4-[3-(1-Ethyl-propyl)-ureido]-2-methoxy-phenoxy}-phenyl)-4-[1-(3-methyl-butyl)-piperidin-4-yloxy]-benzamide,
4-[1-(2-Dimethylamino-ethyl)-piperidin-4-yloxy]-N-(4-{4-[3-(1-ethyl-propyl)-ureido]-2-methoxy-phenoxy}-phenyl)-benzamide,
4-(1-Butyl-piperidin-4-yloxy)-N-(4-{4-[3-(1-ethyl-propyl)-ureido]-2-methoxymethyl-phenoxy}-phenyl)-benzamide,
N-(4-{4-[3-(1-Ethyl-propyl)-ureido]-2-methoxy-phenoxy}-phenyl)-4-[1-(1-methyl-butyl)-piperidin-4-yloxy]-benzamide,
N-(4-{4-[3-(1-Ethyl-propyl)-ureido]-2-methoxy-phenoxy}-phenyl)-4-[1-(Tetrahydro-pyran-4-yl)-piperidin-4-yloxy]-benzamide,
N-(4-{4-[3-(1-Ethyl-propyl)-ureido]-phenoxy}-3-methyl-phenyl)-4-[1-hydroxymethyl-propyl)-piperidin-4-yloxy]-benzamide,
4-(1-Cyclobutyl-piperidin-4-yloxy)-N-{4-[3-(1-ethyl-propyl)-ureido]-phenoxy}-3-methyl-phenyl)-benzamide,
N-(4-{4-[3-(1-Ethyl-propyl)-ureido]-phenoxy}-3-methyl-phenyl)-4-[1-(1-methyl-butyl)-piperidin-4-yloxy]-benzamide,
4-(1-Cyclopentyl-piperidin-4-yloxy)-N-(4-{4-[3-(1-ethyl-propyl)-ureido]-phenoxy}-3-methyl-phenyl)-benzamide,
N-(4-{4-[3-(1-Ethyl-propyl)-ureido]-phenoxy}-3-methyl-phenyl)-4-(1-propyl-piperidin-4-yloxy)-benzamide,
4-(1-Butyl-piperidin-4-yloxy)-N-(4-{4-[3-(1-ethyl-propyl)-ureido]-phenoxy}-3-methyl-phenyl)-benzamide,
N-(4-{4-[3-(1-Ethyl-propyl)-ureido]-phenoxy}-3-methyl-phenyl)-4-(1-methyl-piperidin-4-yloxy)-benzamide,
N-(4-{4-[3-(1-Ethyl-propyl)-ureido]-phenoxy}-3-methyl-phenyl)-4-[1-(3-methyl-butyl)-piperidin-4-yloxy]-benzamide,
4-(1-Ethyl-piperidin-4-yloxy)-N-(4-{4-[3-(1-ethyl-propyl)-ureido]-phenoxy}-3-methyl-phenyl)-benzamide,
N-(4-{4-[3-(1-Ethyl-propyl)-ureido]-phenoxy}-3-methyl-phenyl)-4-(1-isobutyl-piperidin-4-yloxy)-benzamide,
4-(1-Cyclopropyl-piperidin-4-yloxy)-N-(4-{4-[3-(1-ethyl-propyl)-ureido]-phenoxy}-3-methyl-phenyl)-benzamide,
4-(1-Butyl-piperidin-4-yloxy)-N-(4-{4-[3-(1-ethyl-propyl)-ureido]-2-methoxy-phenoxy}-2-fluoro-phenyl)-benzamide,
N-(4-{4-[3-(1-Ethyl-propyl)-ureido]-2-methoxy-phenoxy}-phenyl)-4-(8-methyl-8-aza-bicyclo[3.2.1]oct-(3-endo)-yloxy)-N-propyl-benzamide,
N-(4-{4-[3-(1-Ethyl-propyl)-ureido]-2-methoxy-phenoxy}-2-fluoro-phenyl)-4-(8-methyl-8-aza-bicyclo[3.2.1]oct-(3-endo)-yloxy)-benzamide,
4-(1-Butyl-piperidin-4-yloxy)-N-(4-{4-[3-(1-ethyl-propyl)-ureido]-2-methoxy-phenoxy}-2-fluoro-phenyl)-3-methyl-benzamide,
4-(1-Butyl-piperidin-4-yloxy)-N-{8-[3-(1-ethyl-propyl)-ureido]-10,11-dihydro-dibenzo[b,f]oxepin-2-yl}-benzamide,
N-(4-{4-[3-(1-Ethyl-propyl)-ureido]-2-methoxy-phenoxy}-phenyl)-N-methyl-4-(8-methyl-8-aza-bicyclo[3.2.1]oct-(3-endo)-yloxy)-benzamide,
N-[4-(1-Butyl-piperidin-4-yloxy)-phenyl]-4-{4-[3-(1-ethyl-propyl)-ureido]-2-methoxy-phenoxy}-benzamide,
1-(4-{2-[4-(1-Butyl-piperidin-4-yloxy)-phenyl]-1-methyl-1H-benzoimidazol-5-yloxy}-3-methoxy-phenyl)₃₋₁-ethyl-propyl)-urea,
1-(4-{2-[4-(1-Butyl-piperidin-4-yloxy)-phenyl]-1-propyl-1H-benzoimidazol-5-yloxy}-3-methoxy-phenyl)-3-(1-ethyl-propyl)-urea,
1-(4-{2-[4-(1-Butyl-piperidin-4-yloxy)-phenyl]-1-ethyl-1H-indol-5-yloxy}-3-methoxy-phenyl)-3-(1-ethyl-propyl)-urea,
1-(4-{4-[6-(1-Butyl-piperidin-4-yloxy)-benzooxazol-2-yl]-phenoxy}-3-methoxy-phenyl)-3-(1-ethyl-propyl)-urea,
1-(4-{4-[6-(1-Butyl-piperidin-4-yloxy)-benzooxazol-2-yl]-phenoxy}-phenyl)-3-(1-ethyl-propyl)-urea,
1-(1-Ethyl-propyl)-3-(3-methoxy-4-{4-[5-(1-methyl-piperidin-4-yloxy)-benzofuran-2-yl]-phenoxy}-phenyl)-urea,
4-(1-Butyl-piperidin-4-yloxy)-N-(4-{2-chloro-4-[3-(1-ethyl-propyl)-ureido]-phenoxy}-2-fluoro-phenyl)-3-methyl-benzamide,
4-(1-Butyl-piperidin-4-yloxy)-N-(4-{4-[3-(1-ethyl-propyl)-ureido]-phenoxy}-phenyl)-N-(2-methoxy-ethyl)-3-methyl-benzamide,
4-(1-Butyl-piperidin-4-yloxy)-N-(4-{4-[3-(1-ethyl-propyl)-ureido]-2-methoxy-phenoxy}-phenyl)-N-(2-methoxy-ethyl)-3-methyl-benzamide,
4-(1-Butyl-piperidin-4-yloxy)-N-(4-{4-[3-(1-ethyl-propyl)-ureido]-phenoxy}-2-fluoro-phenyl)-3-methoxy-benzamide,
4-(1-Butyl-piperidin-4-yloxy)-N-(4-{4-[3-(1-ethyl-propyl)-ureido]-3-fluoro-phenoxy}-phenyl)-3-methoxy-benzamide,
N-(4-{2-Ethoxy-4-[3-(1-ethyl-propyl)-ureido]-phenoxy}-phenyl)-3-methyl-4-(1-methyl-piperidin-4-yloxy)-benzamide,
N-(4-{4-[3-(1-Ethyl-propyl)-ureido]-3-fluoro-phenoxy}-phenyl)-3-methyl-4-(1-methyl-piperidin-4-yloxy)-benzamide,
N-(4-{4-[3-(1-Ethyl-propyl)-ureido]-phenoxy}-2-fluoro-phenyl)-3-methyl-4-(1-methyl-piperidin-4-yloxy)-benzamide,
N-(4-{2-Ethoxy-4-[3-(1-ethyl-propyl)-ureido]-phenoxy}-2-fluoro-phenyl)-3-methyl-4-(1-methyl-piperidin-4-yloxy)-benzamide,
4-(1-Butyl-piperidin-4-yloxy)-N-(4-{2-Ethoxy-4-[3-(1-ethyl-propyl)-ureido]-phenoxy}-2-fluoro-phenyl)-benzamide,
4-(1-Butyl-piperidin-4-yloxy)-N-(4-{2-Ethoxy-4-[3-(1-ethyl-propyl)-ureido]-phenoxy}-phenyl)-N-(2-methoxy-ethyl)-benzamide,
4-(1-Butyl-piperidin-4-yloxy)-N-(2-Ethoxy-ethyl)-N-(4-{4-[3-(1-ethyl-propyl)-ureido]-2-methoxy-phenoxy}-phenyl)-benzamide,
4-(1-Butyl-piperidin-4-yloxy)-N-(4-{4-[3-(1-ethyl-propyl)-ureido]-5-fluoro-2-methoxy-phenoxy}-phenyl)-benzamide,
4-(1-Butyl-piperidin-4-yloxy)-N-(4-{4-[3-(1-ethyl-propyl)-ureido]-2,5-difluoro-phenoxy}-phenyl)-benzamide,
1-(1-Methyl-piperidin-4-yl)-1H-indole-5-carboxylic acid (4-{4-[3-(1-ethyl-propyl)-ureido]-2-methoxy-phenoxy}-phenyl)-amide,
1-(1-Methyl-piperidin-4-yl)-1H-indole-5-carboxylic acid (4-{4-[3-(1-ethyl-propyl)-ureido]-phenoxy}-3-methoxymethyl-phenyl)-amide,
1-(1-Butyl-piperidin-4-yl)-1-H-indole-5-carboxylic acid (4-{4-[3-(1-ethyl-propyl)-ureido]-5-fluoro-2-methoxy-phenoxy}-phenyl)-amide,
1-(1-Butyl-piperidin-4-yl-1H-indole-5-carboxylic acid (4-{2-ethoxy-4-[3-(1-ethyl-propyl)-ureido]-phenoxy}-2-fluoro-phenyl)-amide,
1-(1-Butyl-piperidin-4-yl)-1H-indole-5-carboxylic acid (4-{2-ethoxy-4-[3-(1-ethyl-propyl)-ureido]-phenoxy}-phenyl)-amide,
4-(1-Butyl-piperidin-4-yloxy)-N-(4-{4-[3-(1-ethyl-propyl)-ureido]-5-fluoro-2-methoxy-phenoxy}-phenyl)-2,5-difluoro-benzamide,
4-(1-Butyl-piperidin-4-yloxy)-N-(4-{4-[3-(1-ethyl-propyl)-ureido]-2,5-difluoro-phenoxy}-phenyl)-2,5-difluoro-benzamide,
4-(1-Butyl-piperidin-4-yloxy)-N-(4-{2-Ethoxy-4-[3-(1-ethyl-propyl)-ureido]-phenoxy}-phenyl)-2,5-difluoro-benzamide,
4-(1-Butyl-3-fluoro-piperidin-4-yloxy)-N-(4-{2-Ethoxy-4-[3-(1-ethyl-propyl)-ureido]-phenoxy}-phenyl)-benzamide,
4-(1-Dimethylamino-piperidin-4-yloxy)-N-(4-{2-Ethoxy-4-[3-(1-ethyl-propyl)-ureido]-phenoxy}-phenyl)-benzamide,
4-(1-Butyl-pyrrolidin-3-yloxy)-N-(4-{2-Ethoxy-4-[3-(1-ethyl-propyl)-ureido]-phenoxy}-phenyl)-benzamide,
4-[(1-Butyl-piperidin-4-yl)methyl-amino]-N-(4-{4-[3-(1 ethyl-propyl)-ureido]-2-methoxy-phenoxy}-phenyl)-benzamide,
4-[(1-Butyl-piperidin-4-yl)methyl-amino]-N-(4-{4-[3-(1-ethyl-propyl)-ureido]-5-fluoro-2-methoxy-phenoxy}-phenyl)-benzamide,
4-[(1-Butyl-piperidin-4-yl)-methyl-amino]-N-(4-{4-[3-(1-ethyl-propyl)-ureido]-3-fluoro-phenoxy}-phenyl)-benzamide,
4-(1-Butyl-piperidin-4-yloxy)-2-chloro-N-(4-{4-[3-(1-ethyl-propyl)-ureido]-2-methoxy-phenoxy}-phenyl)-benzamide,
1-(1-Butyl-piperidin-4-yl)-2,3-dihydro-1H-indole-5-carboxylic acid (4-{2-Ethoxy-4-[3-(1-ethyl-propyl)-ureido]-phenoxy}-phenyl)-amide,
4-(1-Butyl-piperidin-4-yloxy)-N-(4-{4-[3-(1-ethyl-propyl)-ureido]-2-methoxy-phenoxy}-phenyl)-2-methyl-benzamide,
N-(4-{2-Ethoxy-4-[3-(1-ethyl-propyl)-ureido]-phenoxy}-phenyl)-4-(4-methyl-[1,4]diazépan-1-yl)-benzamide,
N-(4-{2-Ethoxy-4-[3-(1-ethyl-propyl)-ureido]-phenoxy}-phenyl)-4-(4-ethyl-piperazin-1-yl)-benzamide,
1-(1-Butyl-piperidin-4-yl)-1H-indole-5-carboxylic acid (4-{4-[3-(1-ethyl-propyl)-ureido]-phenoxy}-phenyl)-amide,
4-(4-Butyl-piperazin-1-yl)-N-(4-{2-Ethoxy-4-[3-(1-ethyl-propyl)-ureido]-phenoxy}-phenyl)-benzamide,
4-(4-Butyl-[1,4]diazepan-1-yl)-N-(4-{4-[3-(1-ethyl-propyl)-ureido]-5-fluoro-2-methoxy-phenoxy}-phenyl)-benzamide,
4-(4-Butyl-[1,4]diazepan-1-yl)-N-(4-{4-[3-(1-ethyl-propyl)-ureido]-2-methoxy-phenoxy}-phenyl)-benzamide,
4-(1-Butyl-piperidin-4-yloxy)-N-(4{-[3-(1-ethyl-propyl)-ureido]-3-fluoro-phenoxy}-phenyl)-3-methyl-benzamide,
4-(1-Butyl-piperidin-4-yloxy)-N-(4-{2-(2-dimethylamino-ethoxy)-4-[3-(1-ethyl-propyl)-ureido]-phenoxy}-2-fluoro-phenyl)-benzamide,
1-(1-Butyl-piperidin-4-yl)-1H-indole-5-carboxylic acid (4-{4-[3-(1-ethyl-propyl)-ureido]-phenoxy}-3-methyl-phenyl)-amide,
4-(4-Butyl-[1,4]diazepan-1-yl)-N-(4-{2-Ethoxy-4-[3-(1-ethyl-propyl)-ureido]-phenoxy}-phenyl)-benzamide,
4-(1-Butyl-piperidin-4-yloxy)-N-(4-{4-[3-(1-ethyl-propyl)-ureido]-5-fluoro-2-methoxy-phenoxy}-phenyl)-2-fluoro-5-methyl-benzamide,
4-(1-Butyl-piperidin-4-yloxy)-N-(4-{2-Ethoxy-4-[3-(1-ethyl-propyl)-ureido]-phenoxy}-phenyl)-2-fluoro-5-methyl-benzamide,
N-(4-{2-Ethoxy-4-[3-(1-ethyl-propyl)-ureido]-phenoxy}-phenyl)-4-(4-ethyl-piperazin-1-ylmethyl)-benzamide,
1-(1-Butyl-piperidin-4-yl)-1H-indole-5-carboxylic acid (4-{4-[3-(1-ethyl-propyl)-ureido]-2,5-difluoro-phenoxy}-phenyl)-amide,
4-(1-Butyl-piperidin-4-yloxy)-N-(4-{4-[3-(1-ethyl-propyl)-ureido]-2,5-difluoro-phenoxy}-phenyl)-3-methyl-benzamide,
2-Methyl-1,2,3,4-tetrahydro-benzo[4,5]furo[3,2-c]pyridine-8-carboxylic acid (4-{4-[3-(1-ethyl-propyl)-ureido]-2-methoxy-phenoxy}-phenyl)-amide,
4-(1-Butyl-piperidin-4-yloxy)-N-(4-{4-[3-(1-ethyl-propyl)-ureido]-3-fluoro-phenoxy}-3-methyl-phenyl)-benzamide,
4-(4-Butyl-piperazin-1-yl)-N-(4-{2-ethoxy-4-[3-(1-ethyl-propyl)-ureido]-phenoxy}-phenyl)-2-fluoro-5-methyl-benzamide,
4-(1-Butyl-piperidin-4-yloxy)-N-(4-{4-[3-(1-ethyl-propyl)-ureido]-2-methoxy-phenoxy}-phenyl)-N-tetrahydro-pyran-4-yl)-benzamide,
4-(1-Butyl-piperidin-4-yloxy)-N-(4-{4-[3-(1-ethyl-propyl)-ureido]-2-methoxy-phenoxy}-phenyl)-N-(2-methoxy-1-methyl-ethyl)-benzamide,
4-(1-Butyl-piperidin-4-yloxy)-N-(4-{4-[3-(1-ethyl-propyl)-ureido]-2-methoxy-phenoxy}-phenyl)-N-(2-methoxy-propyl)-benzamide,
4-(1-Butyl-piperidin-4-yloxy)-N-(4-{4-[3-(1-ethyl-propyl)-ureido]-2-methoxy-phenoxy}-phenyl)-N-tetrahydro-furan-3-yl)-benzamide,
4-(1-Butyl-piperidin-4-yloxy)-N-(4-{4-[3-(1-ethyl-propyl)-ureido]-2-methoxy-phenoxy}-phenyl)-N-tetrahydro-furan-2-ylmethyl)-benzamide,
4-(1-Butyl-piperidin-4-yloxy)-N-ethyl-N-(4-{4-[3-(1-ethyl-propyl)-ureido]-5-fluoro-2-methoxy-phenoxy}-phenyl)-3-methyl-benzamide,
4-(1-Butyl-piperidin-4-yloxy)-N-(4-{4-[3-(1-ethyl-propyl)-ureido]-5-fluoro-2-methoxy-phenoxy}-phenyl)-N-(2-methoxy-ethyl)-3-methyl-benzamide,
4-(1-Butyl-piperidin-4-yloxy)-N-(4-{2-ethoxy-4-[3-(1-ethyl-propyl)-ureido]-5-fluoro-phenoxy}-phenyl)-3-methyl-benzamide,
4-(1-Butyl-piperidin-4-yloxy)-N-{4-[5-fluoro-4-(3-isopropyl-ureido)-2-methoxy-phenoxy]-phenyl}-3-methyl-benzamide,
4-(1-Butyl-piperidin-4-yloxy)-N-(4-{2-ethoxy-4-[3-(1-ethyl-propyl)-ureido]-5-fluoro-phenoxy}-phenyl)-2-fluoro-5-methyl-benzamide,
(1-Ethyl-propyl)-carbamate of 4-{4-[4-(1-butyl-piperidin-4-yloxy)-3-methyl-benzoylamino]-phenoxy}-3-methoxy-phenyl,
4-(1-Butyl-piperidin-4-yloxy)-N-(4-{5-fluoro-2-methoxy-4-[3-(1-methoxymethyl-propyl)-ureido]-phenoxy}-phenyl)-3-methyl-benzamide,
4-(1-Butyl-piperidin-4-yloxy)-N-(4-{4-[3-(1-ethyl-propyl)-ureido]-5-fluoro-2-methoxy-phenoxy}-phenyl)-3-methyl-benzamide,
N-{4-[5-Fluoro-4-(3-isopropyl-ureido)-2-methoxy-phenoxy]-phenyl}-4-[1-(3-methoxy-propyl)-piperidin-4-yloxy]-3-methyl-benzamide,
4-(4-Butyl-[1,4]diazepan-1-yl)-N-(4-{2-ethoxy-4-[3-(1-ethyl-propyl)-ureido]-phenoxy}-phenyl)-2,5-difluoro-benzamide,
4-(1-Butyl-piperidin-4-yloxy)-N-(4-{4-[3-(1-ethyl-propyl)-ureido]-2-methoxy-phenoxy}-phenyl)-2,5-difluoro-benzamide,
4-[1-(2-Ethoxy-ethyl)-piperidin-4-yloxy]-N-(4-{4-[3-(1-ethyl-propyl)-ureido]-5-fluoro-2-methoxy-phenoxy}-phenyl)-3-methyl-benzamide,
N-(4-{4-[3-(1-Ethyl-propyl)-ureido]-5-fluoro-2-methoxy-phenoxy}-phenyl)-4-[1-(2-methoxy-ethyl)-piperidin-4-yloxy]-3-methyl-benzamide,
N-(4-{4-[3-(1-Ethyl-propyl)-ureido]-5-fluoro-2-methoxy-phenoxy}-phenyl)-4-[1-(3-methoxy-propyl)-piperidin-4-yloxy]-3-methyl-benzamide,
4-(1-Butyl-piperidin-4-ylamino)-N-(4-{2-ethoxy-4-[3-(1-ethyl-propyl)-ureido]-phenoxy}-phenyl)-benzamide,
N-(4-{2-Ethoxy-4-[3-(1-ethyl-propyl)-ureido]-phenoxy}-phenyl)-4-(1 ethyl-piperidin-4-ylamino)-benzamide,
N-(2-Dimethylamino-ethyl)-N-(4-{4-[3-(1-ethyl-propyl)-ureido]-2-methoxy-phenoxy}-phenyl)-4-(1-methyl-piperidin-4-yloxy)-benzamide,
N-(4-{4-[3-(1-Ethyl-propyl)-ureido]-2-methoxy-phenoxy}-phenyl)-4-{1-[3-(tetrahydro-pyran-4-ylamino)-propyl]-piperidin-4-yloxy}-benzamide,
4-[(1-Butyl-piperidin-4-yl)-methyl-amino]-N-(4-{2-ethoxy-4-[3-(1-ethyl-propyl)-ureido]-phenoxy}-phenyl)-benzamide,
4-(1-Butyl-piperidin-4-yloxy)-N-(4-{4-[3-(1-ethyl-propyl)-ureido]-3-fluoro-phenoxy}-3-methyl-phenyl)-3-(2-hydroxy-ethyl)-benzamide,
N-(4-{4-[3-(1-Ethyl-propyl)-ureido]-2-methoxy-phenoxy}-phenyl)-N-(2-methoxy-ethyl)-4-(piperidin-4-yloxy)-benzamide,
N-(4-{4-[3-(1-Ethyl-propyl)-ureido]-2,5-difluoro-phenoxy}-phenyl)-4-(piperidin-4-yloxy)-benzamide,
N-(4-{2-Ethoxy-4-[3-(1-ethyl-propyl)-ureido]-phenoxy}-phenyl)-4-(methyl-piperidin-4-yl-amino)-benzamide,
N-(4-{2-Ethoxy-4-[3-(1-ethyl-propyl)-ureido]-phenoxy}-phenyl)-4-[(1-ethyl-piperidin-4-yl)-methyl-amino]-benzamide,
1-(4-{4-[4-(1-Butyl-piperidin-4-yloxy)-benzoyl]-3,4-dihydro-2H-benzo[1,4]oxazin-7-yloxy}-phenyl)-3-(1-ethyl-propyl)-urea,
N-(4-{4-[3-(1-Ethyl-propyl)-ureido]-3-fluoro-phenoxy}-2-fluoro-phenyl)-4-(1-methyl-piperidin-4-yloxy)-benzamide,
4-(1-Butyl-piperidin-4-yloxy)-N-(4-{4-[3-(1-ethyl-propyl)-ureido]-3-fluoro-phenoxy}-2-fluoro-phenyl)-3-methyl-benzamide,
1-(1-Butyl-piperidin-4-yl)-1H-indole-5-carboxylic acid (4-{4-[3-(1-ethyl-propyl)-ureido]-3-fluoro-phenoxy}-2-fluoro-phenyl)-amide,
1-(4-{9-[4-(1-Butyl-piperidin-4-yloxy)-benzoyl]-6,7,8,9-tetrahydro-5-oxa-9-aza-benzocyclohepten-3-yloxy}-3-methoxy-phenyl)-3-(1-ethyl-propyl)-urea,
4-(1-Butyl-piperidin-4-yloxy)-piperidine-1-carboxylic acid (4-{2-ethoxy-4-[3-(1-ethyl-propyl)-ureido]-phenoxy}-phenyl)-amide,
1-(4-{4-[4-(1-Butyl-piperidin-4-yloxy)-phenoxymethyl]-phenoxy}-3-methoxy-phenyl)-3-(1-ethyl-propyl)-urea,
   and their pharmaceutically acceptable salts, their solvates and hydrates, optical and geometric isomers or their mixtures.

The present invention also relates to compounds of formula (I) chosen from among:
4-(1-Benzyl-piperidin-4-yloxy)-N-(4-{4-[3-(1-ethyl-propyl)-ureido]-5-fluoro-2-methoxy-phenoxy}-phenyl)-3-methyl-benzamide,
N-(4-{4-[3-(1-Ethyl-propyl)-ureido]-5-fluoro-2-methoxy-phenoxy}-phenyl)-3-methyl-piperidin-4-yloxy)-benzamide,
4-(1-Benzyl-piperidin-4-ylamino)-N-(4-{2-ethoxy-4-[3-(1-ethyl-propyl)-ureido]-phenoxy}-phenyl)-benzamide,
N-(4-{2-Ethoxy-4-[3-(1-ethyl-propyl)-ureido]-phenoxy}-phenyl)-4-(piperidin-4-ylamino)-benzamide,
N-(2-Dimethylamino-ethyl)-N-(4-{4-[3-(1-ethyl-propyl)-ureido]-2-methoxy-phenoxy}-phenyl)-4-(piperidin-4-yloxy)-benzamide,
4-[(1-Benzyl-piperidin-4-yl)-methyl-amino]-N-(4-{2-ethoxy-4-[3-(1-ethyl-propyl)-ureido]-phenoxy}-phenyl)-benzamide,
N-(4-{4-[3-(1-Ethyl-propyl)-ureido]-3-fluoro-phenoxy}-3-methyl-phenyl)-3-(2-hydroxy-ethyl)-4-piperidin-4-yloxy)-benzamide,
4-(1-Benzyl-Piperidin-4-yloxy)-N-(4-{4-[3-(1-ethyl-propyl)-ureido]-phenoxy}-3-methyl-phenyl)-benzamide,
N-(4-{4-[3-(1-Ethyl-propyl)-ureido]-2-methoxy-phenoxy}-3-methyl-phenyl)-4-(Piperidin-4-yloxy)-benzamide,
4-(1-Benzyl-Piperidin-4-yloxy)-N-(4-{4-[3-(1-ethyl-propyl)-ureido]-2-methoxy-phenoxy}-3-methyl-phenyl)-benzamide,
N-(4-{4-[3-(1-Ethyl-propyl)-ureido]-2-methoxy-phenoxy}-phenyl)-N-isobutyl-4-(Piperidin-4-yloxy)-benzamide,
N-(4-{4-[3-(1-Ethyl-propyl)-ureido]-2-methoxymethyl-phenoxy}-phenyl), (Piperidin-4-yloxy)-benzamide,
4-(1-Benzyl-Piperidin-4-yloxy)-N-(4-{4-[3-(1-ethyl-propyl)-ureido]-2-methoxymethyl-phenoxy}-phenyl)-benzamide,
   and a pharmaceutically acceptable salt, a solvate and hydrate, optical and geometric isomer or a mixture of these compounds.

The present invention describes different routes of synthesis which are illustrated in schemes 1 to 29 and in the examples, which can be implemented by persons skilled in the art, as indicated in the examples. The starting compounds can be obtained commercially or can be synthesized following the methods described in the literature.

The present application is evidently not limited to any particular method of synthesis and extends to other methods which can be used to produce the indicated compounds.

In the description and examples, the following abbreviations are used:

(BOC)₂O: di-tert-butyl dicarbonate
ACN: acetonitrile
AIBN: azoisobutyronitrile
APCI⁺: atmospheric pressure positive chemical ionisation
AT: ambient temperature
BOC: tertbutyloxycarbonyl
Bzl: benzyl
DCE: 1,2-dichloroethane
DCM: dichloromethane
DIAD: diisopropylazadicarboxylate
DIEA: diisopropylethylamine

### DMA: N,N-dimethylacetamide

### DMAP: N,N-dimethylaminopyridine

### DMF: N,N-dimethylformamide

DMSO: dimethylsulfoxide
EDCI: 1-ethyl-3-(3-dimethylaminopropyl)carbodiimide
ESI⁺: electron spray positive ionisation
HOBT: 1-hydroxybenzotriazole
HPLC: high pressure liquid chromatography
LAH: lithium and aluminium hydride
MeOH: methanol
MS: mass spectrometry
NaH: 60% sodium hydride in mineral oil

### NMP: N-methylpyrollidinone

NH₄OH: ammonium hydroxide (aqueous solution of ammonia)
AP: atmospheric pressure
PPA: polyphosphoric acid
PyClu: Chloro-N,N,N′,N′-bis(tetramethylene)formamidinium hexafluorophosphate
SCX: strong cationic exchange
SNAr: nucleophilic aromatic substitution
SPE: solid phase extraction
TBAF: tetra-n-butylammonium fluoride
TBME: tertbutyl methyl ether
TEA: triethylamine
TFA: trifluoroacetic acid
THF: tetrahydrofurane
TBTU: O-1H-Benzotriazol-1-yl-N,N,N′,N′-tetramethyluronium tetrafluoroborate
TOTU: O-[(ethoxycarbonyl)cyanomethylenamino]-N,N,N′,N′-tetramethyluronium tetrafluoroborate

The compounds of formula (I′) are advantageously prepared according to following SCHEME 1:

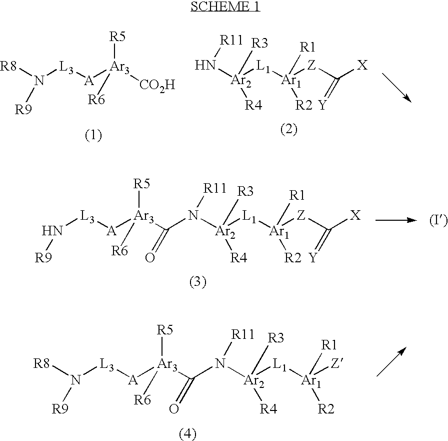

The compounds of formula (V) are advantageously prepared according to following SCHEME 2:

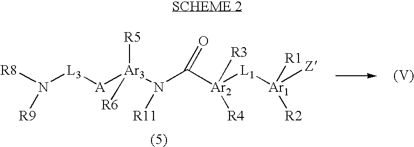

The compounds of formula (VI) are advantageously prepared in accordance with following SCHEMA 3:

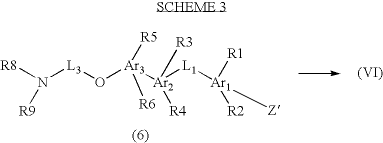

In SCHEMES 1, 2 and 3, X, Y, Z, Ar1, Ar2, Ar3, L1, L2, L3, A and R1 to R11 are such as defined in formula (I).

A further subject of the present invention is a method for preparing the compounds of formula (I′) characterized in that:

a/ amide coupling is conducted between a carboxylic acid (1) an amine (2) of formulas given in SCHEME 1 above, either by in situ activation of the acid (1) using methods known to those skilled in the art, or via an isolated activated species of this acid such as the acid chloride or an activated ester such as the HOBt ester;
b/ or by using conventional N-alkylation reactions in which, in the presence of a base, an amine of formula (3) described in SCHEME 1 is caused to react with a halide of R8-Hal type, Hal advantageously being a chlorine, bromine or iodine atom, and R8 in this case being a (C1-C6)alkyl; (C3-C8)cycloalkyl; (C3-C8)cycloalkyl(C1-C4)alkyl; N,N—(C1-C4)dialkylamino(C2-C6)alkyl; hydroxy(C2-C6)alkyl; (C1-C4)alkoxy(C2-C6)alkyl; (C1-C6)alkoxycarbonyl(C1-C3)alkyl; (C1-C3)alkylcarbonyloxy(C2-C6)alkyl; mono or polyfluoro(C1-C6)alkyl group;
c/ or else, in the presence of a reducer such as sodium borohydride or sodium triacetoxyborohydride, an amine of formula (3) described in SCHEME 1 is caused to react with a suitable aldehyde or ketone following procedures known to persons skilled in the art;
d/ or else, a compound of formula (4), in which Z′ is a-NH₂ group, is converted by causing it to react with an isocyanate, an isothiocyanate or with an amine in the presence of an activator agent such as 1,1′-carbonyldiimidazole or 1,1′-thiocarbonyldiimidazole.

A further subject-matter of the present invention is a method for preparing compounds of formula (V), characterized in that:

e/ a compound of formula (5), in which Z′ is a-NH₂ group, is converted by causing it to react with an isocyanate, an isothiocyanate or with an amine in the presence of an activating agent such as 1,1′-carbonyldiimidazole.

A further subject-matter of the present invention is a method for preparing compounds of formula (VI) characterized in that:

f/ a compound of formula (6), in which Z′ is a —NH₂ group, is converted by causing it to react with an isocyanate or with an amine in the presence of an activating agent such as 1,1′-carbonyldiimidazole.

According to one particular subject-matter, the invention concerns a method for preparing carboxylic acids of formula (1a) or (1a′),

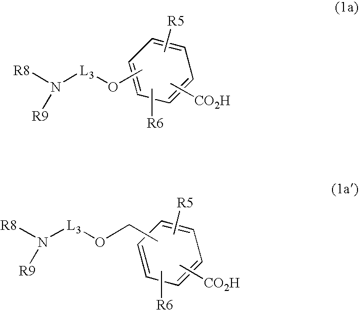

derived from formula (I) in which:
Ar3 is a phenyl radical,
A is an oxygen or a methyleneoxy radical,
L3 represents a (C2-C6)alkylene group; a (C3-C8)cycloalkylene group; bicyclo or polycyclo(C6-C12)alkylene,
R5, R6, R8 and R9 are such as defined in formula (I′).

The carboxylic acids of formula (1a) are advantageously prepared by hydrolysis, in acid or basic medium, of the precursor of carboxylic acid (11a), which is preferably a nitrile or a lower alkyl ester, and which is obtained following the routes indicated in SCHEMES 4a and 4b.

According to SCHEME 4a, pathway 4a.I., the aromatic halogenated derivative (8a) in which Hal advantageously represents a fluorine or a chlorine, undergoes SNAr by an amino alcohol (7a) in a solvent such as DMF, DMA, DMSO or acetone in the presence of a base such as NaH, Na₂CO₃, K₂CO₃, Cs₂CO₃ at temperatures lying between −65° C. and 150° C., for 2 h to 72 h. According to pathway 4a.II., the amino alcohol (7a) is caused to react with the phenol (9) in the presence of DIAD and triphenylphosphine in an anhydrous solvent such as THF at temperatures lying between −78° C. and 60° C., for 2 h to 72 h, following the Mitsunobu Reaction [Hughes, Org. React., 42, 1992, p. 335<<The Mitsunobu Reaction>>]. According to pathway 4a.III., the phenol (9) is caused to react with a chlorohalogenated alkyl derivative (32) in which Hal advantageously represents a chlorine, bromine or iodine atom, in a solvent such as DMF, DMA, DMSO or acetone in the presence of a base such as NaH, Na₂CO₃, K₂CO₃, Cs₂CO₃ at temperatures lying between −20° C. and 150° C., for 2 h to 72 h. The chlorinated derivate (9′) thus obtained is treated with a suitable amine in a solvent such as DMF, DMA, NMP, THF, ACN or acetone in the presence of a base such as TEA, DIEA or K₂CO₃ at temperatures lying between 0° C. and 100° C., for 1 h to 96 h to afford (11a).

According to SCHEME 4b, (11a) is obtained from the intermediate (11b) by deprotection of the protecting group PG following procedures known to those skilled in the art, PG preferably being a BOC, a benzyl or a phthalimide, followed by reductive amination or N-alkylation. (11b) is obtained along the following pathways:
pathway 4b.I.: the aromatic halogenated derivative (8a) undergoes SNAr by an amino alcohol (7b), for which PG represents a protecting group preferably BOC, benzyl or phthalimide.
pathway 4b.II.: the amino alcohol (7b) reacts according to a Mitsunobu Reaction with the phenol (9).
pathway 4b.III.: the amino alcohol (7b) is converted into a methanesulfonate derivative following procedures known to those skilled in the art, followed by reaction with the phenol (9) in a solvent such as DMF, DMA, DMSO or acetone in the presence of a base such as NaH, Na₂CO₃, K₂CO₃, Cs₂CO₃ at temperatures ranging from −20° C. to 150° C., for 2 h to 72 h.

(11a) can also be obtained following the pathway indicated in SCHEME 4c: the alcohol (7b) is caused to reacted with the phenol (9′) according to a Mitsunobu Reaction. The intermediate (11c) thus obtained is treated with succinimide iodide in an acid medium, leading to the iodized amine (1d) which, after reductive amination or N-alkylation, gives the derivative (11e). Treatment of (11e) with copper cyanide in a solvent such as DMF under reflux leads to the intermediate (11a).

The carboxylic acids of formula (1a′) are advantageously prepared following the pathway indicated in SCHEME 4d by hydrolysis, in acid or basic medium, of the precursor of carboxylic acid (11a′), which is preferably a nitrile or a lower alkyl ester: the amino alcohol (7b) is first caused to react with the halogenated benzyl derivative (8c) in which Hal preferably represents a chlorine or bromine, in a solvent such as DMF, DMA, DMSO or acetone in the presence of a base such as NaH, Na₂CO₃, K₂CO₃, Cs₂CO₃ at temperatures lying between −20° C. and 150° C., from 2 h to 72 h. The protecting group PG of the intermediate (11f) thus obtained is deprotected using the procedures applied for (11b), followed by reductive amination or N-alkylation of the released amine, to afford (11a′).

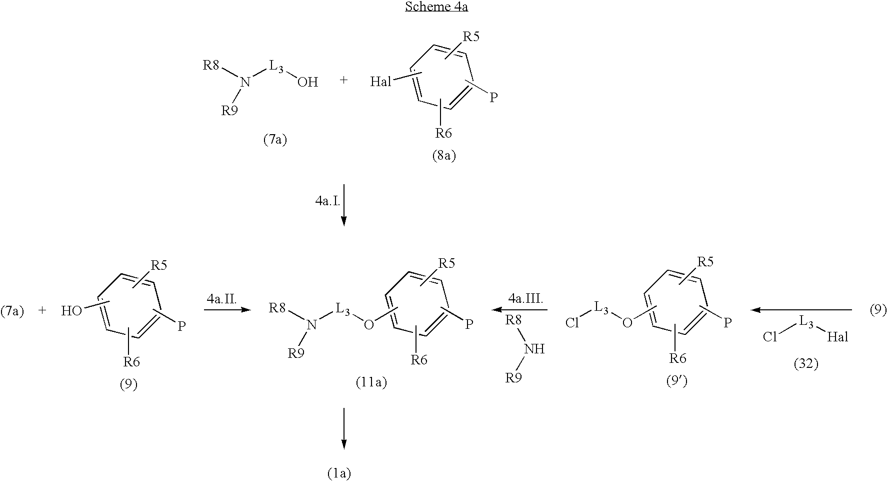

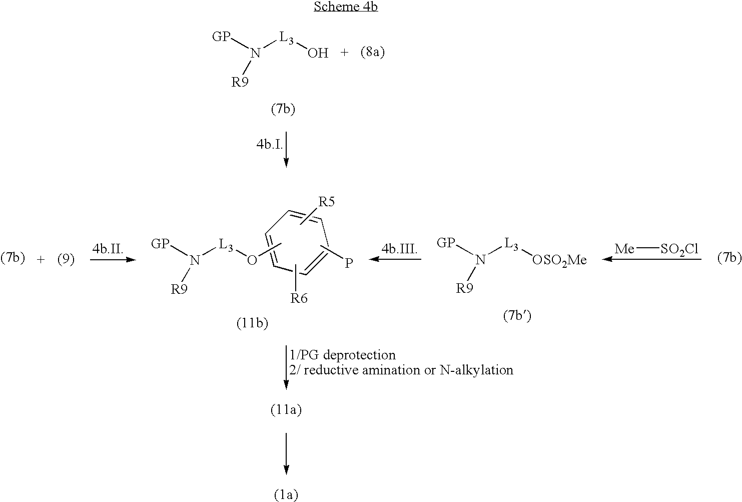

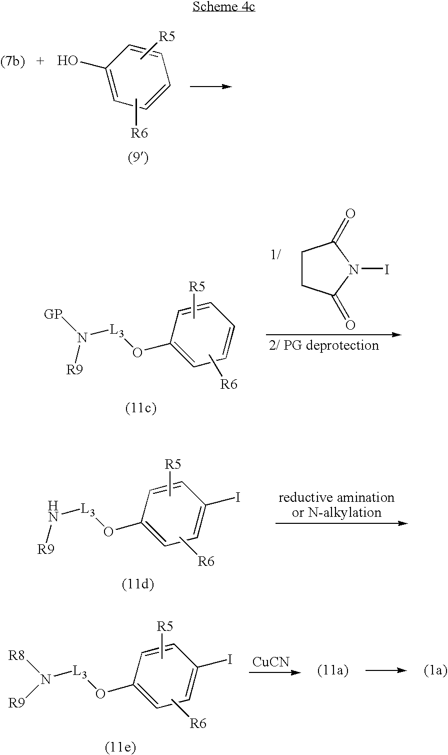

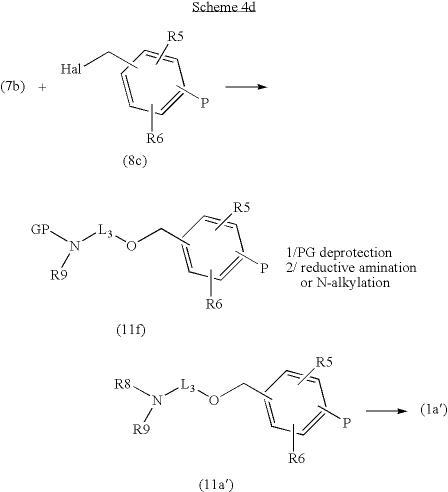

### Schemes 4

According to another particular subject-matter, the invention concerns a method for preparing carboxylic acids of formula (1b) or (1b′),

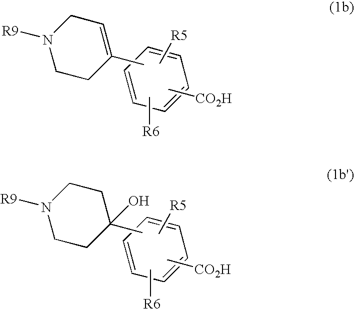

derived from formula (I) in which:
Ar3 is a phenyl radical,
A is a simple bond,
the group:

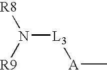

represents:

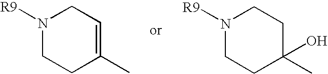

R5, R6 and R9 are such as defined in formula (I)

The carboxylic acids of formula (1b) are advantageously prepared following the different pathways indicated in SCHEMES 5 and 6, starting with the key oxazoline intermediate (16). The oxazoline intermediate (14) is obtained by peptide coupling of 2-amino-2-methyl-1-propanol with a benzoic acid of type (12) following procedures known to those skilled in the art to produce the amide (13) which is cyclized in the presence of excess thionyl chloride at between 0° C. and 100° C., for 1 h to 72 h, in the presence or absence of an inert organic solvent. The oxazoline (16) is then obtained by treatment of (14) with a magnesium derivative prepared, using methods known to those skilled in the art, from a commercially available ketone of type (15), in an inert solvent of THF type, at between entre 0° C. and 100° C., from 1 h to 24 h.

According to SCHEME 5, the amine function of the intermediate (16), protected by a benzyl group (Bzl), is initially released by catalytic hydrogenation, preferably in the presence of a catalyst of Pd on charcoal type, in an inert solvent such as ethyl acetate, in the presence or absence of acetic acid, at ambient pressure or under high pressure, between 0° C. and 100° C., from 1 h to 24 h; in a second phase, the released amine function reacts with:
a suitable aldehyde or ketone in the presence of a reducer such as sodium borohydride or sodium triacetoxyborohydride in an inert solvent of DCM, chloroform, dichloroethane or acetonitrile type, in the presence or absence of an acid such as acetic acid, at temperatures lying between 0° C. and 100° C., from 1 h to 96 h, following a reductive amination reaction;
an alkyl halide, preferably an alkyl iodide, bromide or chloride, in a solvent such as DMF, DMA, NMP, THF, ACN or acetone in the presence of a base such as TEA, DIEA or K₂CO₃ at temperatures lying between 0° C. and 100° C., from 1 h to 96 h;
a commercially available, activated carboxylic acid, or prepared extemporaneously or in situ following procedures known to those skilled in the art.
   to afford the intermediate (17).

The unsaturated carboxylic acids (1b) are obtained from (17) following the 3 alternative pathways described in SCHEME 5:
following pathway 5.I., directly by dehydration and at the same time full hydrolysis of the oxazoline function of (17);
in sequence following pathway 5.II., by partial hydrolysis of the oxazoline function to obtain the amide (18), which is then subjected to hydrolysis at the same time as dehydration;
in sequence following pathway 5.III., the intermediate (17) first undergoing dehydration of the alcohol function, followed by complete hydrolysis of the oxazoline function.

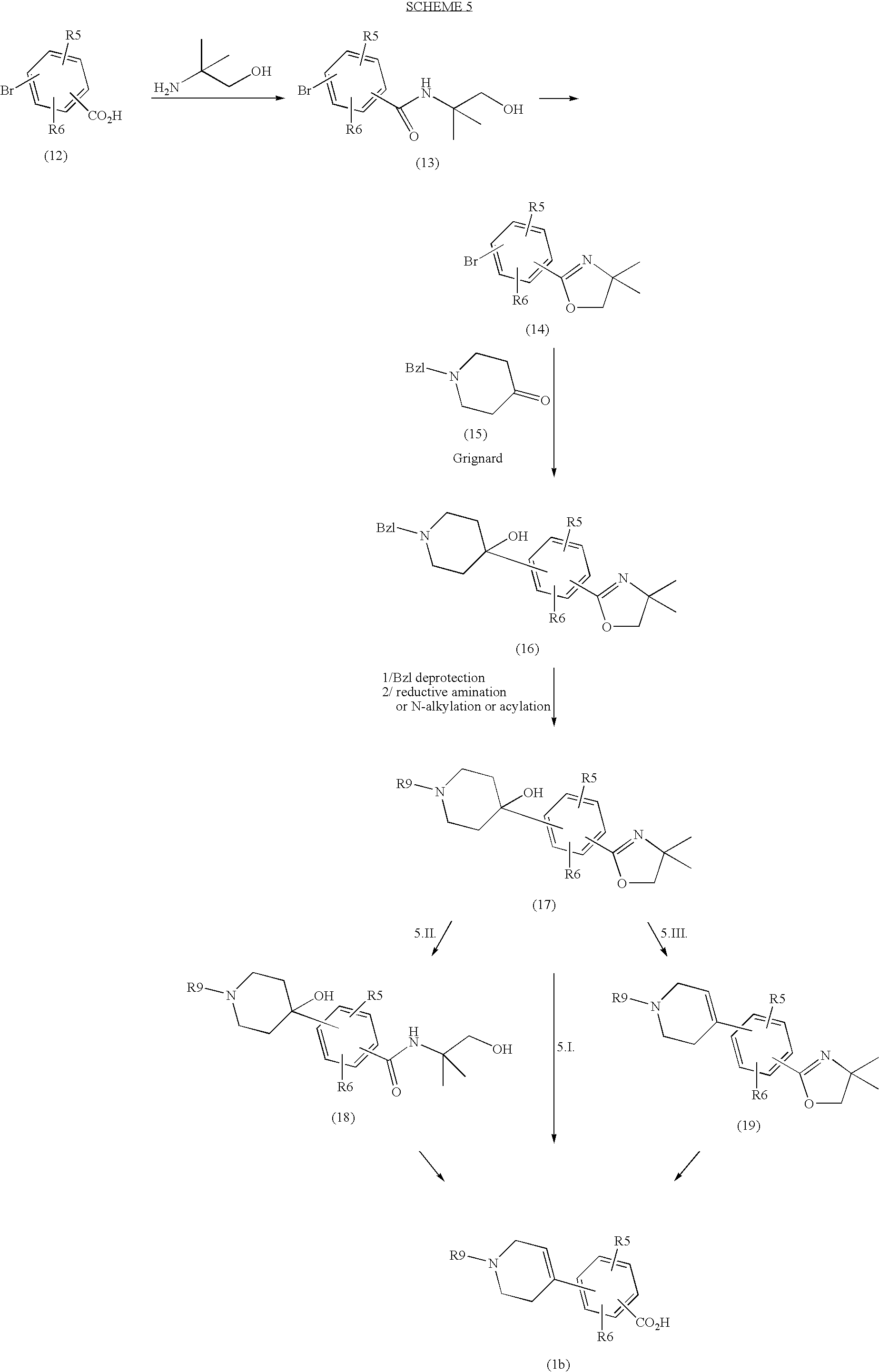

According to SCHEME 6, the carboxylic acids (1b) and (1b′) are prepared from an ester key intermediate (20) which is obtained by hydrolysis of the oxazoline (16) in an acid medium, preferably via excess H₂SO₄ in a solvent of alcohol type, preferably ethanol, at temperatures lying between 0° C. and 100° C., followed by deprotection of the benzyl function under the same conditions as those described for (16). The intermediate (20) leads to acids (1b) or (1b′) following the 3 pathways described in SCHEME 6:
following pathway 6.I., (1b) is obtained by adding function R9 under the same conditions as those described to obtain (17), followed by dehydration and at the same time hydrolysis of the ester (21);
following pathway 6.II., the intermediate (20) first undergoes dehydration followed by addition of the R9 function, under the same conditions as those described for (17), and by hydrolysis of the unsaturated ester (22);
following pathway 6.III., (1b′) is obtained by hydrolysis of the ester (21).

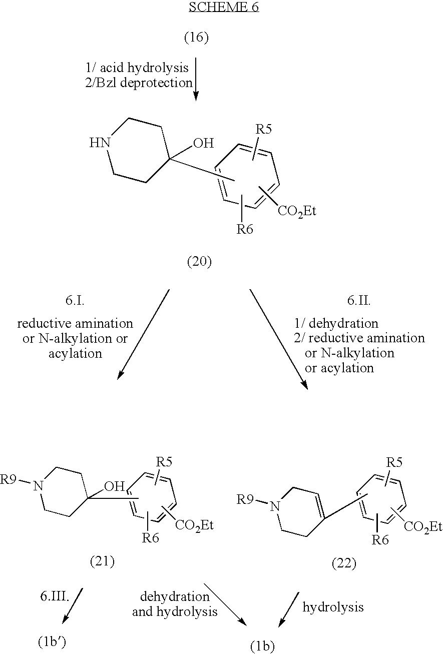

A further particular subject-matter of the invention concerns a method for preparing carboxylic acids of formula (1c),

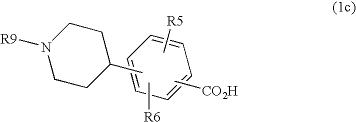

derived from formula (1) in which:
Ar3 is a phenyl radical,
A is a simple bond,
the group:

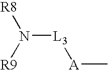

represents:

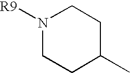

R5, R6 and R9 are such as defined in formula (I).

The carboxylic acids of formula (1c) are advantageously prepared following the pathway indicated in SCHEME 7, starting with the key oxazoline intermediate (16). The dehydration and hydrolysis of (16) and the deprotection of the benzyl of intermediate (23) by hydrogenation under the conditions described for (16), lead to the saturated ester (24). The addition of the R9 function is made by reductive amination, N-alkylation or acylation of the amine (24) under the conditions described to obtain (17). In this manner the ester (25) is obtained, which is hydrolysed leading to acid (1c).

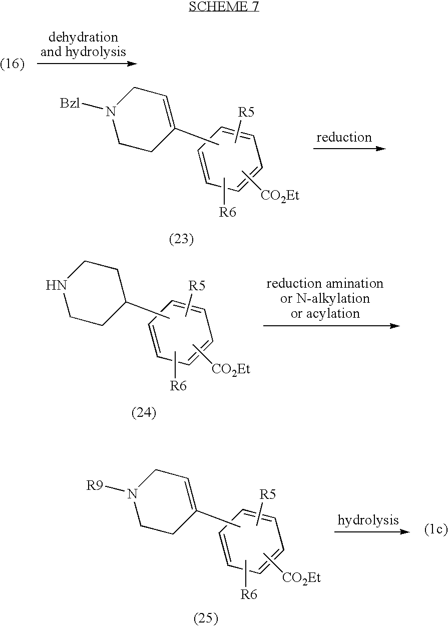

A further particular subject-matter of the invention concerns a method for preparing carboxylic acids of formula (1d) or (1d′),

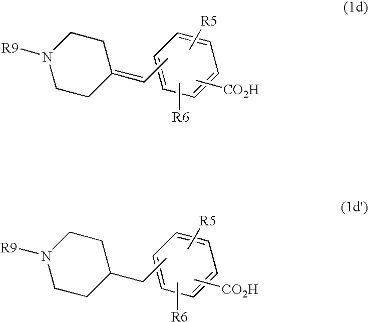

derived from formula (1) in which:
Ar3 is a phenyl radical,
A is a (C1-C3)alkylene or (C2-C3)alkylidene group,
the group:

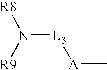

represents:

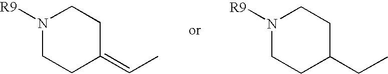

R5, R6 and R9 are such as defined in formula (I).

The carboxylic acids of formula (1d) and (1d′) are advantageously prepared following the pathway indicated in SCHEME 8, starting with a key phosphite intermediate (27), obtained by treatment with triethylphosphite at 160° C., without any solvent, of a benzyl bromide (26). (27) is then caused to react with a N-alkylpiperidone (28) in a basic medium at temperatures close to 0° C. and in an inert atmosphere in the presence of an anhydrous solvent such as THF, to produce the unsaturated ester (29). The acids (1d) are obtained by hydrolysis in an acid or basic medium of the ester (29), whereas the acids (1d′) are obtained from acids (1d) by hydrogenation at atmospheric pressure in solvents such as methanol, ethyl acetate or THF, in the presence of a suitable catalyst, preferably palladium on charcoal, at AT for 1 h to 24 h.

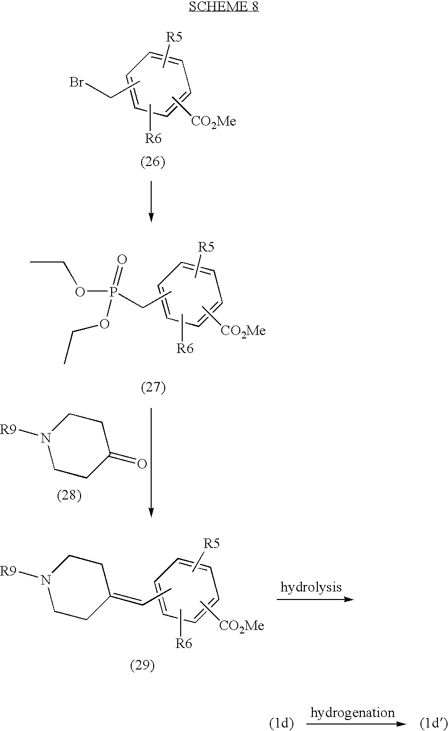

A further particular subject-matter of the invention concerns a method for preparing carboxylic acids of formula (1e), (1e′) or (1e″),

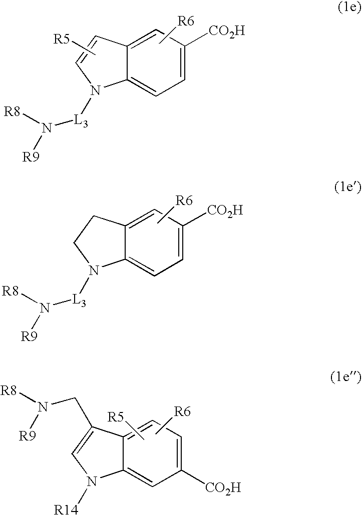

derived from formula (I) in which:
Ar3 is an indolyl ou indolynyl group,
A is a simple bond,
the group:

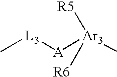

represents:

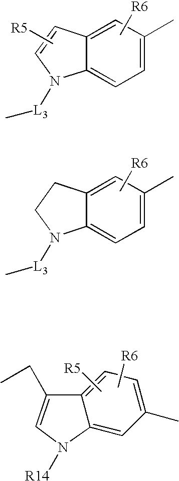

L3 is a (C2-C6)alkylene radical,
R5, R6, R8, R9 and R14 are such as defined in formula (I).

The carboxylic acids of formula (1e) are advantageously prepared following the pathways indicated in SCHEME 9a, from the key methyl ester intermediate of an 1H-Indole-5-carboxylic acid (30a). Following pathway 9.I., the precursor ester (31a) is obtained by deprotonating the NH function of the indole (30a) by action of a base such as NaH at ambient temperature for 30 min to 2 h, in a solvent such as THF, DMF, DMA or DMSO, followed by alkylation with an aliphatic halogenated derivative of formula (10) at temperatures lying between 50° C. and 150° C., for 1 h to 24 h. Following pathway 9.II., the NH function of the indole (30a) is first alkylated, under the above-described conditions, by a chlorohalogenated alkyl derivative (32) in which Hal preferably represents a chlorine, bromine or iodine atom. The 1-chloroalkylindole (33) thus obtained is caused to react with an amine in the presence of a base such as pyridine, TEA or DIEA, in a solvent such as THF, DMF, DMA or DMSO, at between 50° C. and 150° C., for 3 h to 72 h to afford the ester (31) which, after acid or basic hydrolysis, leads to carboxylic acid (1e).

The carboxylic acids (1e) and (1e′) for which the

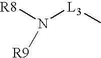

group represents a piperidine of type

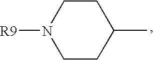

are advantageously prepared following SCHEME 9b: the aniline (30b) is caused to react with a piperidone (28) under a reductive amination reaction to yield the intermediate (30c), which is cyclized into indole (31b) in a highly acid medium. The carboxylic acid (1e) is then obtained by hydrolysis of the ester function of (31b). Also, the indole (31b) is first reduced to indoline (31c) in the presence of a reducer such as sodium cyanoborohydride in acetic acid; the ester function of (31c) is then hydrolysed to afford the carboxylic acid (1e′).

The carboxylic acids of formula (1e″) are advantageously prepared following the pathway indicated in SCHEME 10, from the key methyl ester intermediate of a 1H-Indole-6-carboxylic acid (34). Initially, the indolic NH— is alkylated, following the procedure described for (31a), by an alkyl halide (35) in which Hal advantageously represents the chlorine, bromine or iodine atom. The alkylated indole (36) thus obtained is caused to react with a suitable amine in the presence of formaldehyde and acetic acid at temperatures lying between 0° C. and 50° C., for 1 h to 24 h, to generate the precursor ester (37) which, after acid or basic hydrolysis, leads to the carboxylic acid (1e″).

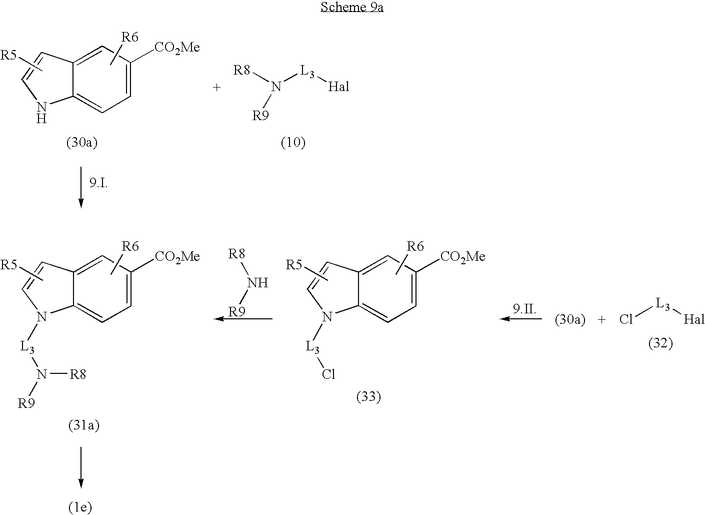

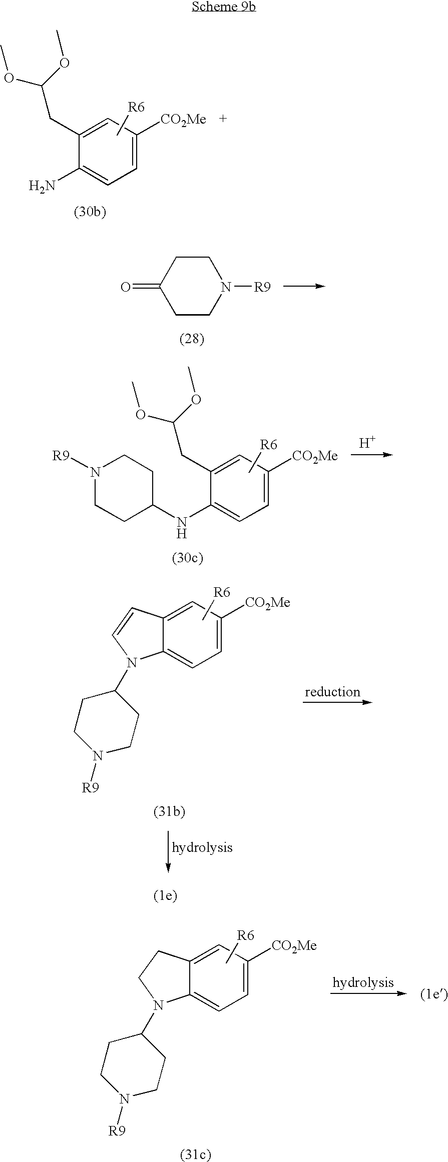

### Schemes 9

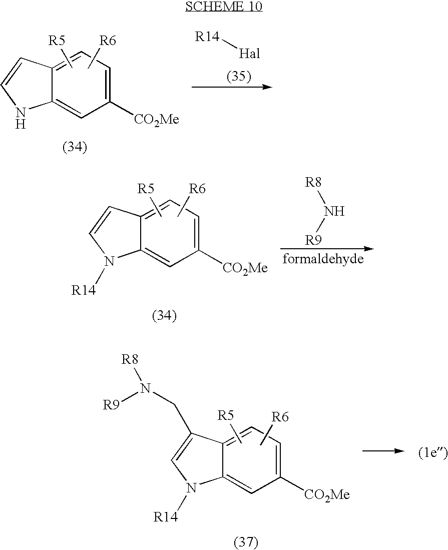

A further particular subject-matter of the invention concerns a method for preparing carboxylic acids of formula (1f),

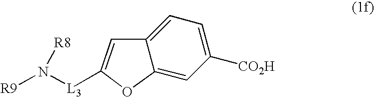

derived from formula (1) in which:
Ar3 is a benzofuranyl group,
R5 and R6 are hydrogen atoms,
A is a simple bond,
the group:

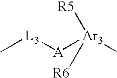

represents:

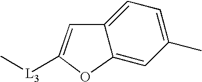

L3 is a (C1-C6)alkylene radical,
R8 and R9 are such as defined in formula (I).

The carboxylic acids of formula (1f) are advantageously prepared following the pathway indicated in SCHEME 11. The key alkynamine intermediate (39), obtained by substitution with a suitable mesylate (38), is caused to react with an iodized phenol (40) in DMF in the presence of tetramethyl-1,1,3,3-guanidine, triphenylphosphine palladium chloride (II) and copper iodide, at temperatures lying between 0° C. and 100° C., for 1 h to 72 h, to produce the ester (41) which, by acid or basic hydrolysis, leads to the acid (1f).

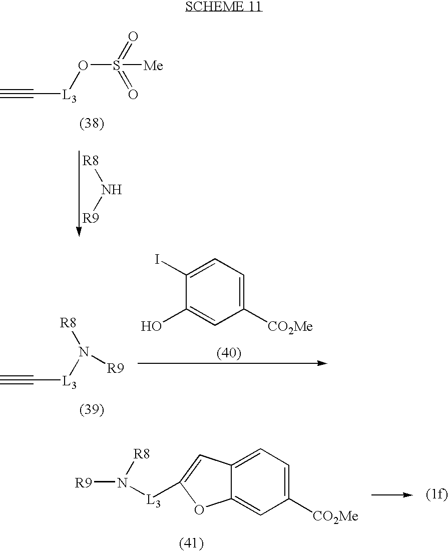

A further particular subject-matter of the invention concerns a method for preparing carboxylic acids of formula (1 g),

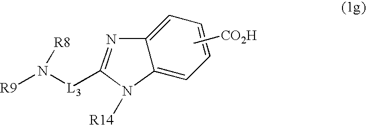

derived from formula (1) in which:
Ar3 is a benzimidazolyl group,
R5 and R6 are hydrogen atoms,
A is a simple bond,
the group:

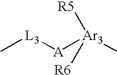

represents:

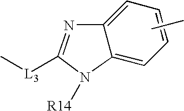

L3 is a (C1-C6)alkylene radical,
R8, R9 and R14 are such as defined in formula (I).

The carboxylic acids of formula (1 g) are advantageously prepared following the pathway indicated in SCHEME 12: the nitrohalogenated benzoic acid (42) in which Hal preferably represents a chlorine or fluorine atom, is subjected to SNAr by a suitable amine in a solvent such as DMF, DMA, DMSO or acetone in the presence of a base such as TEA or DIEA at temperatures lying between 0° C. and 150° C., for 2 h to 72 h, followed by hydrogenation of the nitro function in the presence of a catalyst of Raney Ni type or Pd on charcoal, in a solvent such as THF, MeOH, ethanol, methoxyethanol, DCM or DMF, at ambient temperature for 2 h to 24 h, to produce orto phenylenediamine (43). The primary amine function (43) is acylated by an aminoacid of type (44) following procedures known to those skilled in the art. The monoacylated orto phenylenediamine (43′) thus obtained is cyclized in benzimidazole in an aqueous hydrochloric acid medium in the presence of an alcohol, preferably ethanol, and diethyl ether at temperatures lying between 0° C. and 100° C., for 2 h to 72 h. Under these conditions, the ester (45) is obtained, which is hydrolysed to afford the acid (1 g).

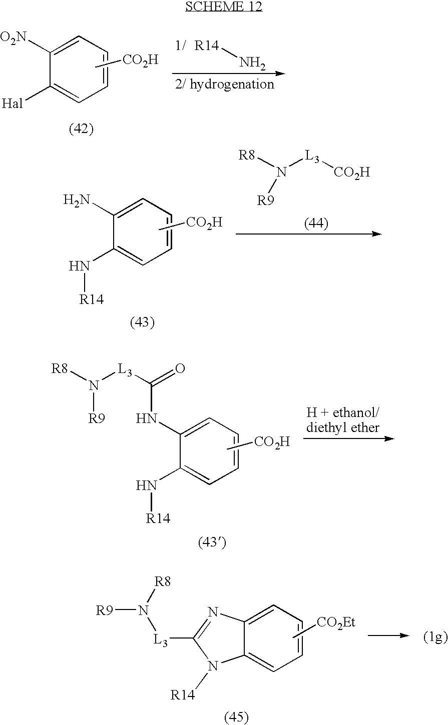

A further particular subject-matter of the invention concerns a method for preparing carboxylic acids of formula (1 h-Aa), (1 h-Ab), (1 h-Ac) or (1 h-Ad)

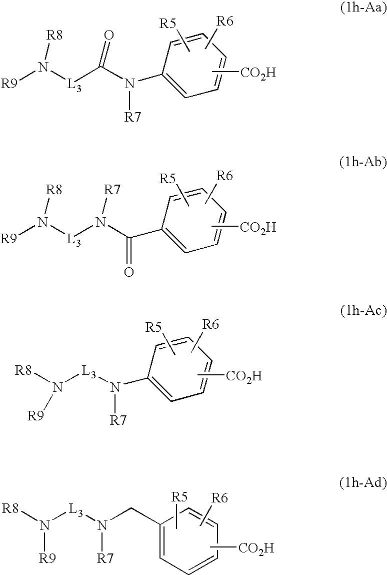

derived from formula (1) in which:
Ar3 is a phenyl,
A represents one of the groups below:

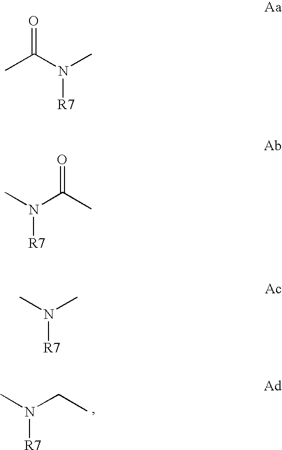

L3 is a (C2-C6)alkylene radical,
R5 to R9 are such as defined in formula (I).

The carboxylic acids of formula (1 h-Aa) are advantageously prepared following the pathways indicated in SCHEME 13:

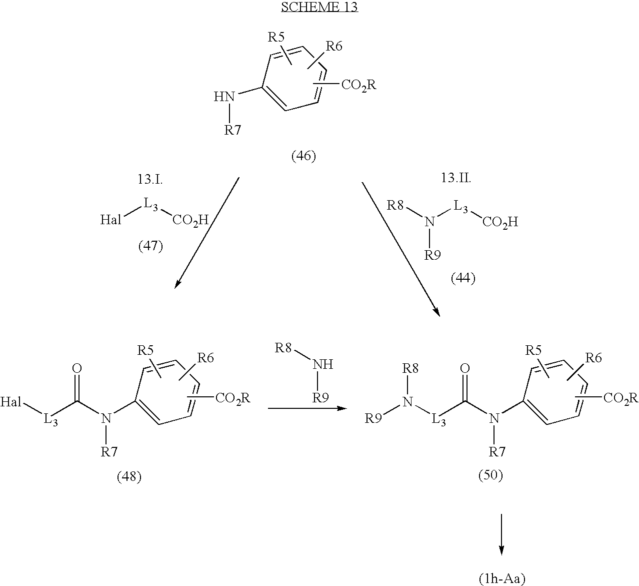

along pathway 13.I., the amine function of the ester of aminobenzoic acid (46) in which R is a lower alkyl, is acylated by a halogenated aliphatic acid (47) in which Hal preferably represents a chlorine, bromine or iodine atom. The halogenated derivative (48) thus obtained is caused to react with a suitable amine in the presence of a base such as pyridine, TEA or DIEA, in a solvent such as THF, DMF, DMA or DMSO, at between 50° C. and 150° C., for 3 h to 72 h to generate the ester (50) which, after hydrolysis, leads to the acid (1 h-Aa),
along pathway 13. II., the ester (50) is obtained directly by acylation of the aniline (46) with an aminoacid of type (44).

The carboxylic acids of formula (1 h-Ab) are advantageously prepared according to SCHEME 14, by coupling a protected monophtalic acid (51) with a suitable diamine (52), followed by hydrolysis of the ester (53).

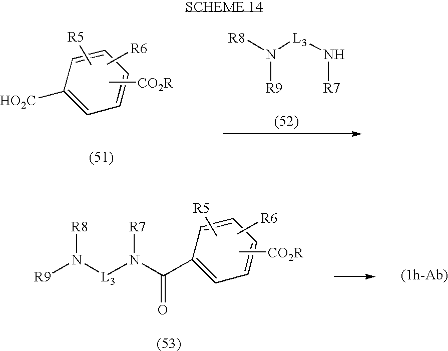

The carboxylic acids of formula (1 h-Ac) and (1 h-Ad) are advantageously prepared following the pathways indicated in SCHEMES 15. In SCHEME 15a, they are obtained from a halogenated derivative (54) in which Hal is preferably a chlorine or fluorine atom and P is a precursor of carboxylic acid, preferably a lower alkyl nitrile or ester:
along pathway 15.I., when R7 represents a hydrogen atom or a (C1-C6)alkyl group, the key intermediate (54) undergoes SNAr by a diamine of type (52a) in a solvent such as DMF, DMA, DMSO or acetone in the presence of a base such as NaH, Na₂CO₃, K₂CO₃, Cs₂CO₃ at temperatures lying between −65° C. and 150° C., for 2 h to 72 h, followed by hydrolysis of the P group of derivative (55a) thus obtained, to afford the carboxylic acid (1 h-Ac). (55a) can also be obtained along pathway 15.II.: the key intermediate (54) undergoes SNAr by an amine of type (52b) for which PG is preferably a methyl or BOC group, followed by deprotection of PG and reductive amination or N-alkylation of the released amine function, leading to the intermediate (55a);
along pathway 15.III., when R7 represents (C1-C6)alkylcarbonyl group: the key intermediate (54) undergoes SNAr by a diamine of type (56), followed by acylation of the secondary aniline with a carboxylic acid R19-CO₂H, R19 representing a (C1-C6)alkyl radical, to generate the intermediate (58). The P group of (58) is hydrolysed, leading to carboxylic acid (1 h-Ac).

According to SCHEME 15b, an amine of type (55c), obtained following procedures known to those skilled in the art, is initially treated with succinimide iodide in an acid medium and then with copper cyanide in a solvent such DMFunder reflux, to generate the nitrile (SSd), which is hydrolysed in an acid or basic medium leading to carboxylic acid (1 h-Ac).

According to SCHEME 15c, a diamine of type (52a) is caused to react with the halogenated benzyl derivative (8c) in which Hal preferably represents a chlorine or bromine, in a solvent such as DMF, DMA, DMSO, acetone or ethanol in the presence of a base such as NaH, Na₂CO₃, K₂CO₃, Cs₂CO₃ at temperatures lying between −20° C. and 150° C., for 2 h to 72 h. The P function of derivative (55e) thus obtained is then hydrolysed in an acid or basic medium to afford acid (1 h-Ad).

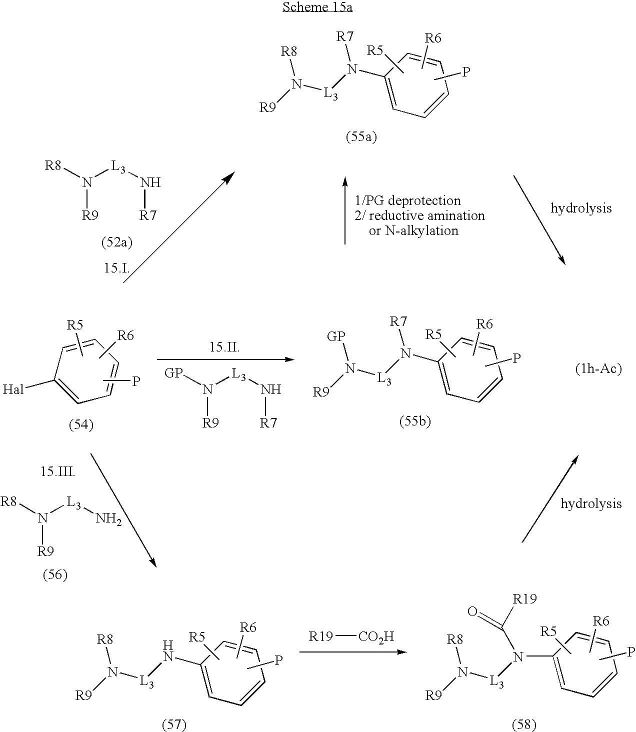

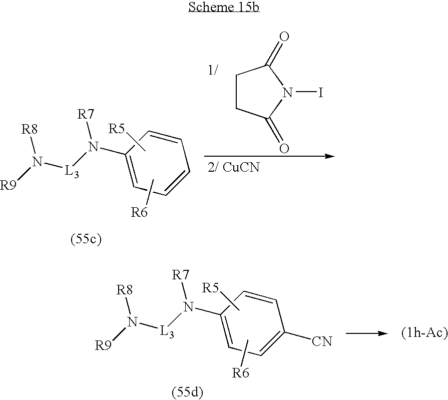

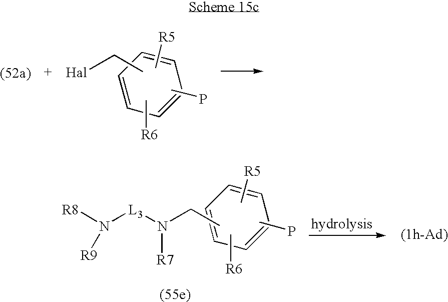

### Schemes 15

According to a further particular subject-matter, the invention concerns a method for preparing amines of formula (2a) or (2a′),

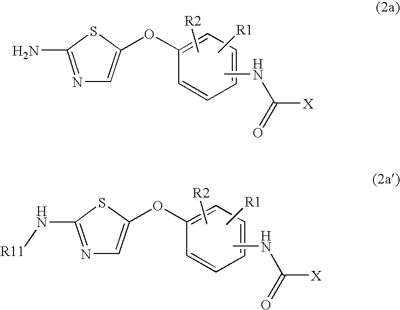

derived from formula (2) in which:
Ar1 is a phenyl,
Ar2 is a thiazole,
Y and L1 are oxygen atoms,
Z is a NH radical,
X represents a N—(C1-C6)alkylamino group,
R1 to R4 and R11 are such as defined in formula (I)

The amines (2a) are advantageously prepared following the pathways indicated in SCHEME 16, from the key phenoxythiazole intermediate (60), obtained by reaction of 2-amino-5-bromothiazole with a nitrophenol (59), in a solvent such as DMF, DMA, DMSO or acetone in the presence of a base such as NaH, Na₂CO₃, K₂CO₃, Cs₂CO₃ at temperatures lying between −20° C. and 150° C., for 2 h to 72 h:
along pathway 16.I., the nitro function of (60) is hydrogenated to amine (61), under the conditions described to obtain (43). The treatment of (61) with an isocyanate or an aminoacylimidazole, prepared extemporaneously or in situ, by reaction of a suitable amine or hydrazine in the presence of 1,1′-carbonyldiimidazole (CDI), in an inert solvent such as THF, at temperatures lying between −20° C. and 60° C., for 3 h to 120 h, leads to (2a).
along pathway 16.II., the amine function of (60) is protected with a BOC group using procedures known to those skilled in the art, and then the nitro group is hydrogenated to afford (62). The amine function of (62) is caused to react with an isocyanate or suitable amine or hydrazine in the presence of CDI under the same conditions as those described for (61). The BOC group is then deprotected in an acid medium to produce (2a).

In SCHEME 16 the radicals R12 and R13 are such as defined for general formula (I), R20 represents a (C1-C6)alkyl group optionally substituted by a (C1-C3)alkoxy(C1-C3)alkyl group, and R21 and R22, the same or different, represent a hydrogen atom or a (C1-C6)alkyl group optionally substituted by a (C1-C3)alkoxy(C1-C3)alkyl group.

The amines (2a′) are advantageously prepared from amines (2a), following the pathway indicated in SCHEME 16: the amine function of (2a) is acylated by a suitable carboxylic acid, R1′-CO₂H, R11′ being a (C1-C5)alkyl radical, and the amide function of the intermediate thus obtained is reduced in the presence of excess LAH in an anhydrous solvent such as THF, at temperatures lying between 0° C. and 80° C., for 12 h to 72 h.

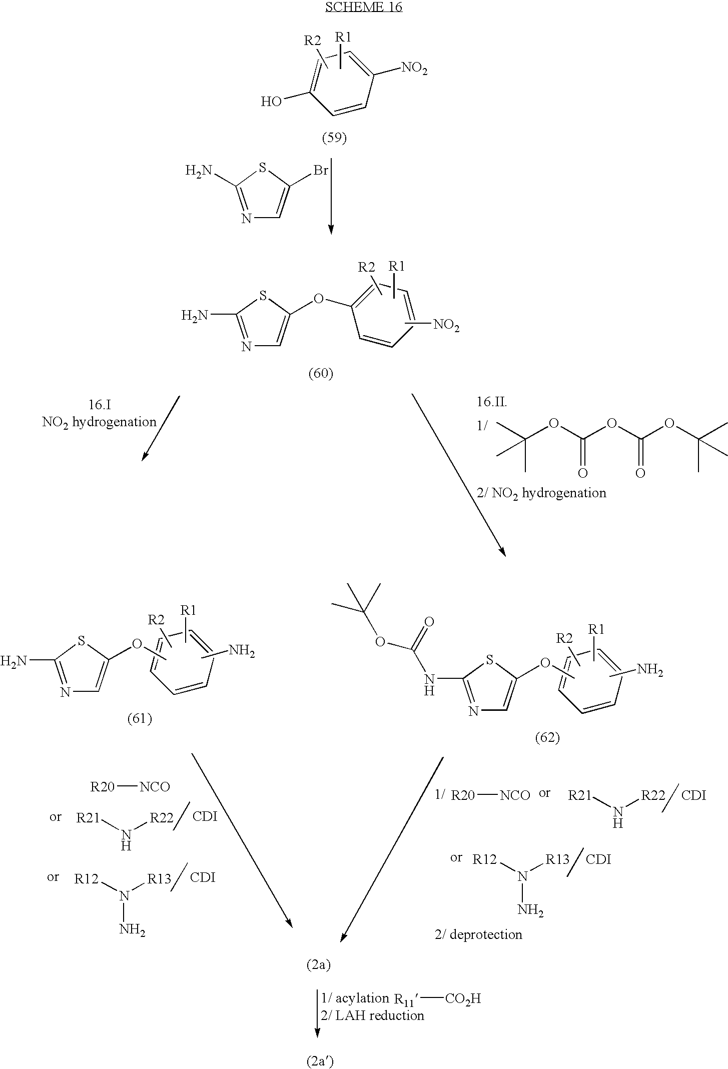

A further particular subject-matter of the invention concerns a method for preparing amines of formula (2b) or (2b′),

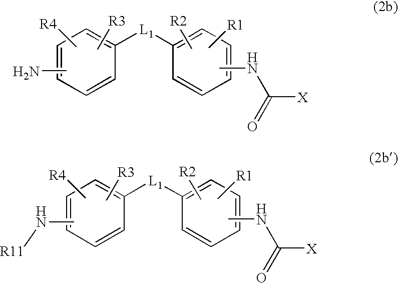

derived from formula (2) in which:
Ar1 and Ar2 are phenyl radicals,
Y is an oxygen atom,
L1 is an oxygen or sulfur atom,
Z is a NH group,
X, R1 to R4 and R11 are such as defined in formula (I).

The amines (2b) and (2b′) are advantageously prepared following the pathways indicated in SCHEMES 17a-d and 18, from the key intermediates (65), (68a), (68a′) and (66b):
According to SCHEME 17a, (65) is treated with an R20-NCO isocyanate or with an aminoacylimidazole under conditions described for (61); or else (65) is acylated with a R23-CO₂H carboxylic acid, R23 being a N,N—(C1-C6)dialkylamino(C1-C3)alkyl radical. The BOC protecting group is then deprotected to afford (2b).
According to SCHEME 17b, (68a) is caused to react with an isocyanate or an aminoacylimidazole under the conditions described for (61), or else it is acylated with a R23-CO₂H carboxylic acid, followed by hydrogenation to produce the aniline (2b).
According to SCHEME 17c, (2b) is obtained from intermediate (68a), in which R2 is a phenol function protected by the protecting PG, preferably a methyl. The phenol is first deprotected under conditions known to those skilled in the art, followed by protection with a BOC group of the aniline (68b) thus obtained, and by alkylation of the phenol with a halogenated derivative R2′-Hal, Hal preferably being a chlorine or bromine atom, and R2′ being a (C1-C6)alkyl, (C1-C3)alkoxy(C2-C3)alkyl or N,N—(C1-C3)dialkylamino(C2-C3)alkyl radical. Two alternative routes are then followed to produce (2b) from the intermediate (68c) thus obtained: along pathway 17c.I., the BOC protecting group is first deprotected, followed by reaction of the aniline function thus released with an R20-NCO isocyanate, with an aminoacylimidazole or with a R23-CO₂H carboxylic acid, such as described for Schemes 17a et 17b, and finally by hydrogenation of the nitro function; along pathway 17c.II., the nitro function is first hydrogenated, followed by reaction of the aniline function thus released with an R20-NCO isocyanate, with an aminoacylimidazole or with a R23-CO₂H carboxylic acid, such as described in Schemes 17a and 17b, and finally by deprotection of the BOC protecting group.
According to SCHEME 17d, the urea (66c) is obtained by treatment of the aniline (66b) with a R20-NCO isocyanate or with an aminoacylimidazole under the conditions described for (61). (66c) then reacts with a halogenated derivative (67) in which Hal preferably represents a chlorine or fluorine atom, in a solvent such as DMF, DMA, DMSO or acetone in the presence of a base such as NaH, Na₂CO₃, K₂CO₃, Cs₂CO₃ at temperatures lying between −20° C. and 150° C., for 2 h to 72 h, followed by hydrogenation of the nitro group under the conditions described to obtain (43).
The secondary aniline (2b′) is obtained from (2b) following two alternative routes: along pathway 18.I., (2b) is acylated by a R11″-CO₂H carboxylic acid, R11″ being a (C1-C5)alkyl or (C1-C6)alkoxy(C2-C5)alkyl radical, and the amide bond of (65′) thus obtained is reduced in the presence of LAH, under the conditions described to obtain (2a′); along pathway 18.II., (2b′) is obtained directly by N-alkylation of aniline (2b) with a halogenated derivative R11-Hal in a solvent such as DMF, DMA, DMSO or acetone in the presence of a base such as NaH, Na₂CO₃, K₂CO₃, Cs₂CO₃ at temperatures lying between −20° C. and 150° C., for 2 h to 72 h, Hal preferably being a chlorine, bromine or iodine atom; along pathway 18.II., (2b′) is also obtained by reaction of the aniline (2b) with a mesilate R11-OSO₂Me or by reaction with a suitable ketone or aldehyde (R11′R11″)C═O, (R11′R111″)C═ being a precursor of the R11 group such as defined in general formula I, under conditions known to those skilled in the art.

The intermediate (65) is obtained as indicated in Scheme 17a by reaction of a phenol or thiol (63) with a halogenated derivative (64) in which Hal preferably represents a chlorine or fluorine atom, in a solvent such as DMF, DMA, DMSO or acetone in the presence of a base such as NaH, Na₂CO₃, K₂CO₃, Cs₂CO₃ at temperatures lying between −20° C. and 150° C., for 2 h to 72 h, followed by hydrogenation of the nitro group under the conditions described to obtain (43).

The intermediate (68a) is obtained as described in Scheme 17b pathway 17b.I., by reaction of a phenol or thiol (66a) and a halogenated derivative (67) in a solvent such as DMF, DMA, DMSO or acetone in the presence of a base such as NaH, Na₂CO₃, K₂CO₃, Cs₂CO₃ at temperatures lying between −20° C. and 150° C., for 2 h to 72 h, followed by deprotection of the BOC group. Alternatively, following pathway 17b.II. (68a) is obtained from derivative (66b), non-protected on the aniline function, and from the halogenated derivative (67), such as described above for the reaction of (66a).

In SCHEMES 17a-c and 18 the radicals R11, R12 and R13 are such as defined in general formula (I), L1 is an oxygen or sulfur and R20 to R22 are such as defined in SCHEME 16.

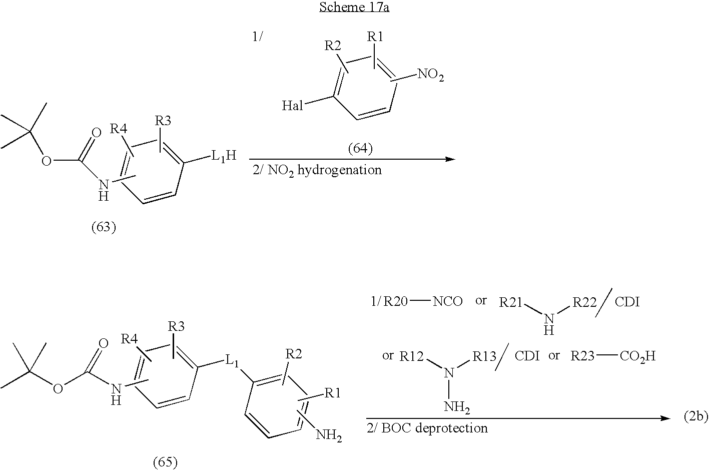

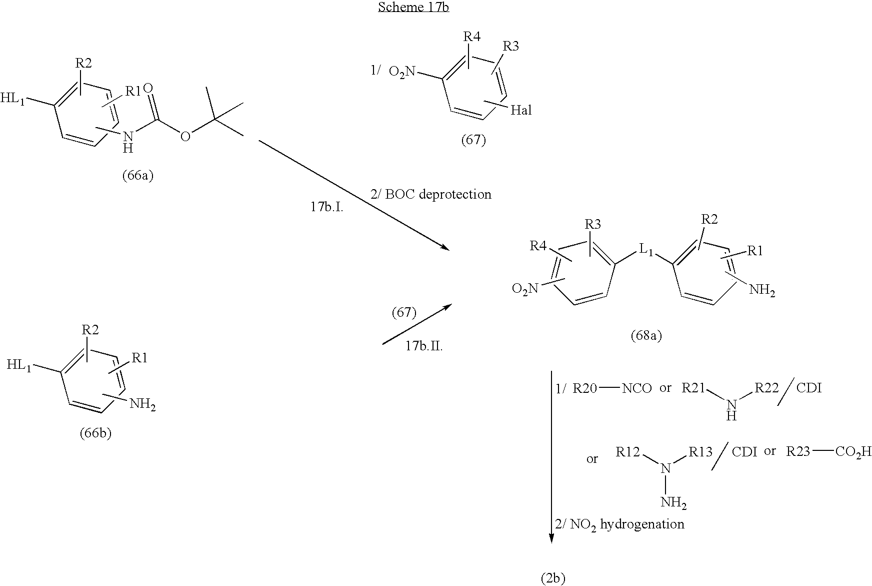

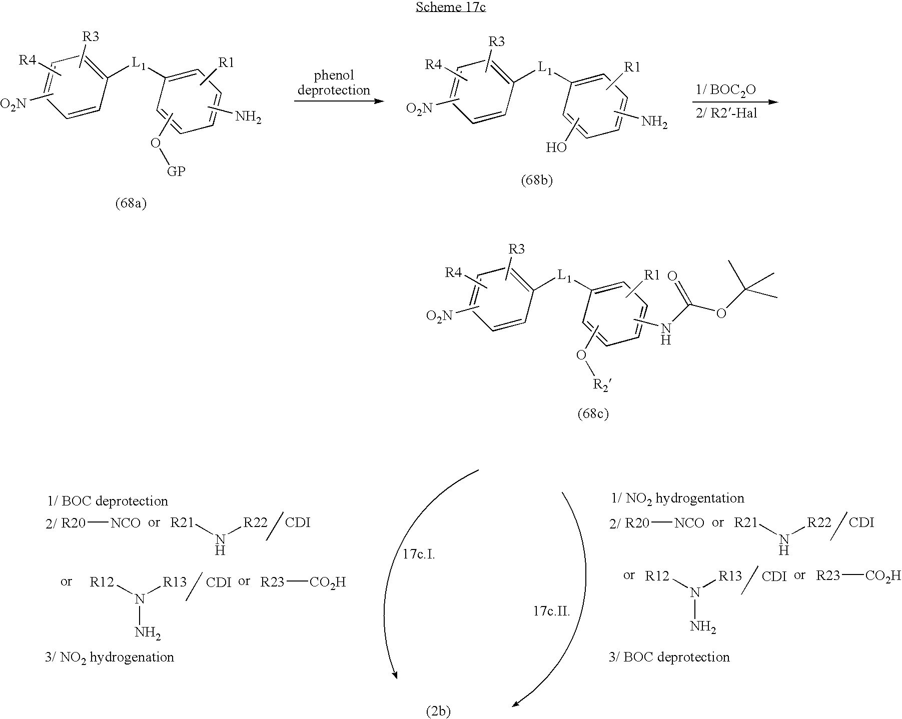

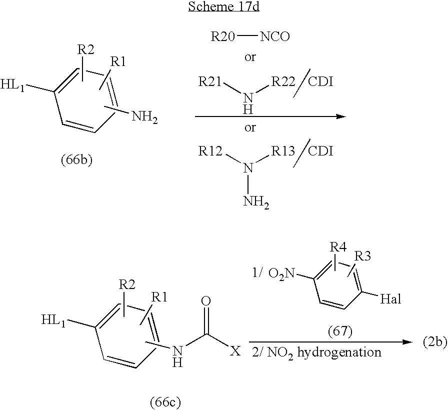

### Schemes 17

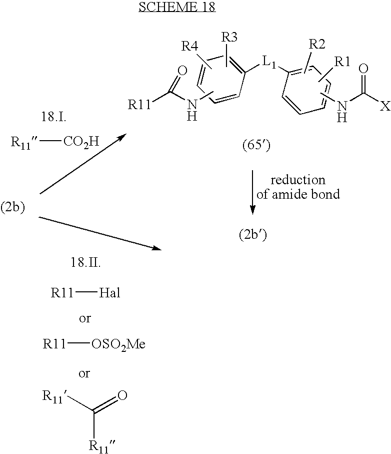

A further particular subject of the invention concerns a method for preparing amines of formula (2c),

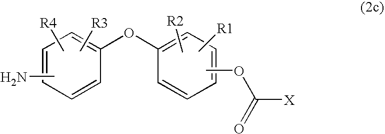

derived from formula (2) in which:
Ar1 and Ar2 are phenyl radicals,
Y, Z and L1 are oxygen atoms,
X is a N—(C1-C6)alkylamino group,
R11 is a hydrogen,
R1 to R4 are such as defined in formula (I).

The amines (2c) are advantageously prepared following the pathway indicated in SCHEME 19, from the key intermediate (70): the chloromethylenecarbonate derivative (71), obtained by treatment of (70) with chloromethyl chloroformate in a solvent such as THF or DCM, at temperatures lying between −20° C. et 60° C., for 1 h to 24 h, is caused to react with a suitable amine to obtain a carbamate derivative, after hydrogenation of the nitro function under the conditions described to obtain (43), leading to aniline (2c).

The key intermediate (70) is obtained as described in SCHEME 19 by reaction of a phenol of type (69) with a halogenated derivative (67), in a solvent such as DMF, DMA, DMSO or acetone in the presence of a base such as NaH, Na₂CO₃, K₂CO₃, Cs₂CO₃ at temperatures lying between −20° C. and 150° C., for 2 h to 72 h, followed by deprotection of the methoxy group in an acid medium, preferably concentrated HBr or pyridine hydrochloride at temperatures lying between 20° C. and 190° C., 1 h to 15 h. The radicals R21 and R22 are such as defined for SCHEME 16.

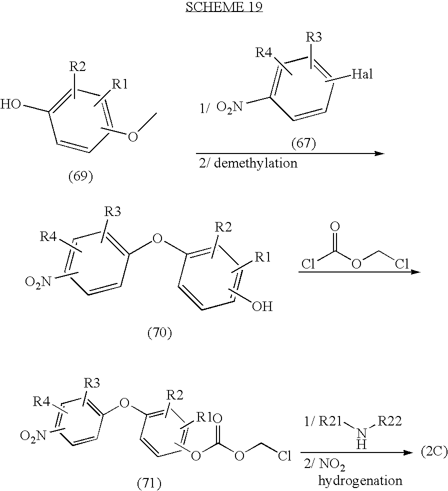

A further particular subject-matter of the invention concerns a method for preparing amines of formula (2d),

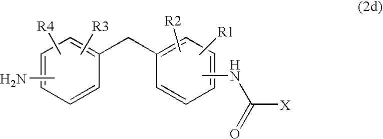

derived from formula (2) in which:
Ar1 and Ar2 are phenyl radicals,
X is a N—(C1-C6)alkylamino group,
Y is an oxygen atom,
Z is a NH radical,
L1 is a methylene,
R1 is a hydrogen,
R1 to R4 are such as defined in formula (I).

The amines (2d) are advantageously prepared following the pathway indicated in SCHEME 20: the methylenedianiline (72), mono-protected by a commercially available BOC group, or obtained using methods known to those skilled in the art, is treated with an isocyanate R20-NCO or an aminoacylimidazole under the conditions described for (61), followed by deprotection of the BOC group, to afford (2d).

In SCHEME 20, the radicals R12 and R13 are such as defined in general formula (I) and R20 to R22 are such as defined for SCHEME 16.

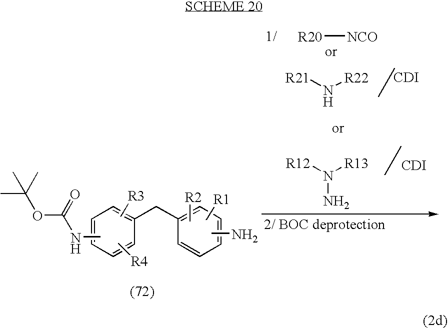

A further particular subject-matter of the invention concerns a method for preparing amines of formula (3a) or (3a′),

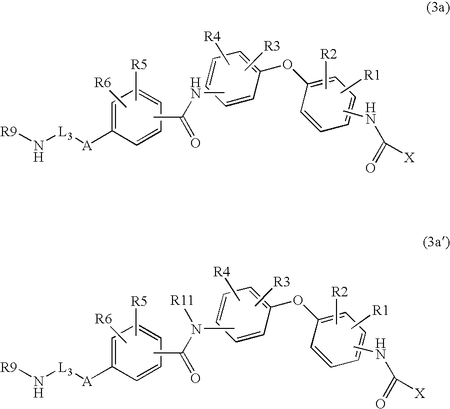

derived from formula (3) in which:
Ar1, Ar2 and Ar3 are phenyl radicals,
X is a N—(C1-C6)alkylamino group optionally substituted by a (C1-C3)alkoxy(C1-C3)alkyl group,
Y is an oxygen atom,
Z is a NH radical,
L1 is an oxygen atom,
A is an oxygen atom or NH radical,
L3 preferably represents a (C2-C6)alkylene group; a (C3-C8)cycloalkylene group optionally substituted by one or more (C1-C3)alkyl groups, by a hydroxy group or by a (C1-C3)alkoxy group; bicyclo ou polycyclo(C6-C12)alkylene,
R9, R1 to R6 and R11 are such as defined in formula (I).

The amines (3a) and (3a′) are advantageously prepared as indicated in SCHEMES 21 and 22:
According to SCHEME 21, the amide coupling of an aniline of type (2b) is conducted, in which L1 is preferably an oxygen, with the key aminoacid intermediate (77) protected on the amine function by a protecting group PG, which preferably represents a benzyl or BOC. The protecting group PG of the intermediate (78) thus obtained is deprotected following procedures known to those skilled in the art, to produce (3a).
According to SCHEME 22, the key intermediate (77) is caused to react under an acylation reaction with a nitroaniline (82). The amide (83) thus obtained is alkylated by a halogenated derivative R11-Hal in which Hal advantageously represents a bromine or iodine atom, in a solvent such as DMF, DMA, DMSO or acetone in the presence of a base such as NaH, Na₂CO₃, K₂CO₃ or Cs₂CO₃ at temperatures lying between −20° C. and 150° C., for 2 h to 120 h, and the nitro function is then hydrogenated to the amine under the conditions described to obtain (43). The intermediate (84) is treated with an isocyanate R20-NCO or an aminoacylimidazole under the conditions such as described for (61) and the protected amine (85) thus obtained is deprotected to produce the compound (3a′). In SCHEME 22, PG advantageously represents a BOC group and R20 to R22 are such as defined for SCHEME 16.

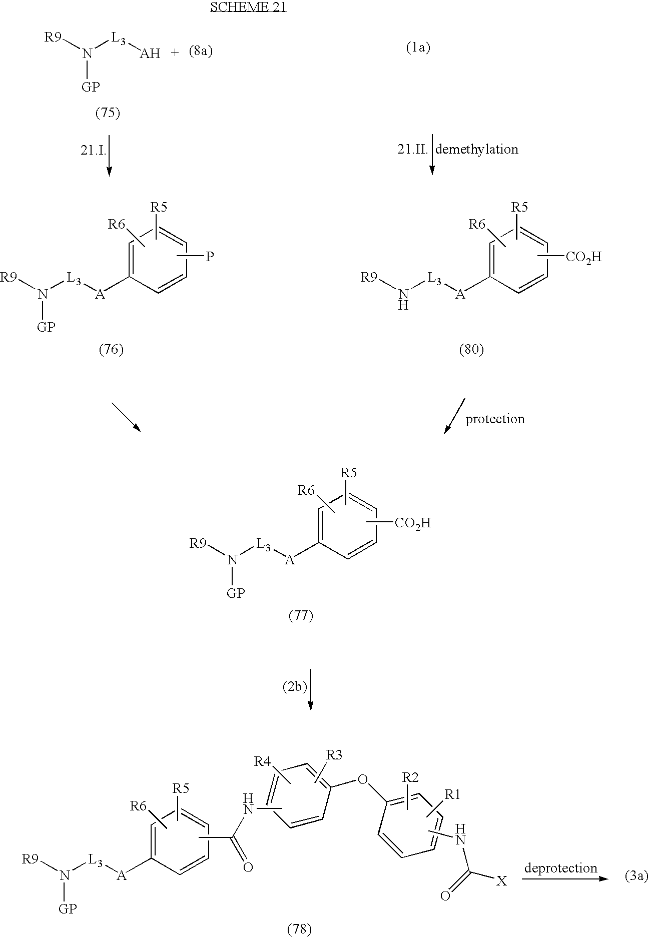

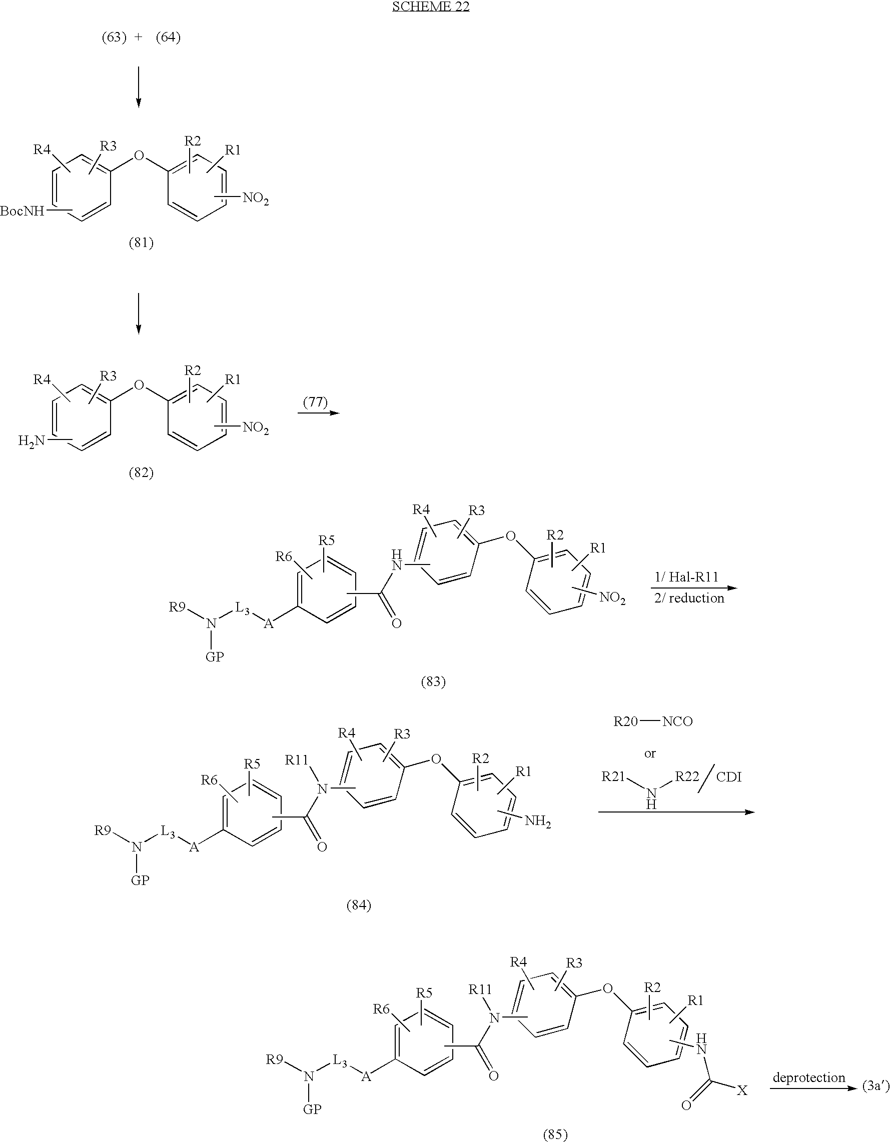

The intermediate (77) is advantageously prepared following the two pathways described in SCHEME 21:
along pathway 21.I., the derivative (75), for which A preferably represents an oxygen or a NH radical, protected on the amine function by a protecting group PG, preferably benzyl or BOC, reacts with an aromatic halogenated derivative (8a) in a solvent such as DMF, DMA, DMSO or acetone in the presence of a base such as NaH, Na₂CO₃, K₂CO₃, Cs₂CO₃, DIEA or TEA, at temperatures ranging from −20° C. to 150° C., for 2 h to 72 h. The P function, P being a precursor of carboxylic acid, preferably a lower alkyl nitrile or ester, of the intermediary (76) thus obtained is hydrolysed with acid (77)
along pathway 21.II., an aminoacid of type (1a), for which R8 is a methyl, is demethylated following procedures described in the literature (see for example Boja et al J. Med. Chem., 37, 1994, p. 1220) and the amine (80) thus obtained, in which A is an oxygen, is protected by a protecting group PG, preferably a BOC, under conditions known to those skilled in the art.

The nitroaniline (82) is advantageously prepared following the pathway indicated in SCHEME 22, by reaction of the compounds (63) and (64) such as described in SCHEME 17, followed by deprotection of the BOC protecting group of the intermediate (81) thus obtained.

A further particular subject-matter of the invention concerns a method for preparing amines of formula (4a) or (4a′),

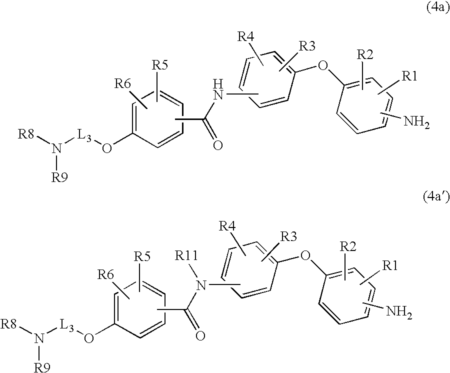

derived from formula (4) in which:
Ar1, Ar2 and Ar3 are phenyl radicals,
Z′ is a NH₂ radical,
L1 and A are oxygen atoms,
The group:

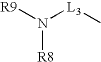

represents:

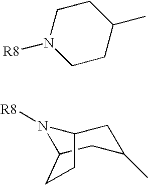

R8, R11 and R1 to R6 are such as defined in formula (I)

The amines (4a) and (4a′) are advantageously prepared as indicated in SCHEMES 23 and 24:
according to SCHEME 23, an acid of type (1a) reacts under amide coupling with an aniline aniline (82), followed by hydrogenation of the nitro group to obtain the aniline (4a)
according to SCHEME 24, an acid (77), for which A is an oxygen and PG is advantageously a BOC group, reacts under amide coupling with an aniline (82) and the amide (87) thus obtained is alkylated by a halogenated derivative R11-Hal, in accordance with the procedure described for SCHEME 22, followed by deprotection of the protecting group PG. The amine function of (88) is N-alkylated by an alkyl halide, preferably chloride, bromide or iodide, or reacts by reductive amination on a suitable aldehyde or ketone, following the procedures described for SCHEME 1, and the nitro group is then hydrogenated to produce the aniline (4a′). In SCHEME 24, R8′-CH═ et R8′R8″-C═ are precursors of the R8 group, such as defined in formula (I′).

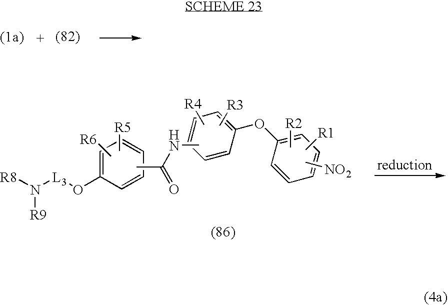

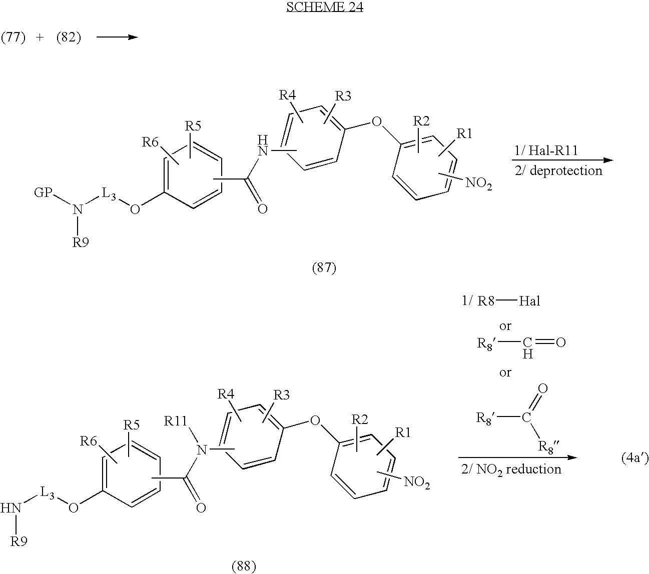

A further particular subject-matter of the invention concerns a method for preparing amines of formula (5a),

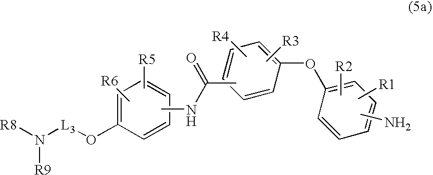

derived from formula (5) in which:
Ar1, Ar2 and Ar3 are phenyl radicals,
Z′ is a NH₂ radical,
L1 and A are oxygen atoms,
the group:

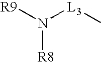

represents:

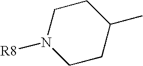

R11 is the hydrogen atom
R8 and R1 to R6 are such as defined in formula (I).

The amines (5a) are advantageously prepared as indicated in SCHEME 25:
along pathway 25.I., the aniline (90) is obtained from an aromatic nitrohalogenated derivative (89), which undergoes SNAr by the amino alcohol (7a), followed by catalytic hydrogenation of the nitro group,
along pathway 25.II, the carboxylic acid (92) is obtained from an aromatic halogenated nitrile derivative (8′), which undergoes SNAr by the nitrophenol (91), followed by hydrolysis of the nitrile group,
the amide coupling of compound (90) with (92) generates the nitro derivative (93) which, by hydrogenation of the nitro group, leads to derivative (5a)
   (89), (8′) and (91) are commercially available or obtained using procedures known to those skilled in the art; the different reactions involved in SCHEME 25 are conducted in accordance with protocols already described for the preceding schemes; for (89) and (91) Hal preferably represents a chlorine or fluorine atom.

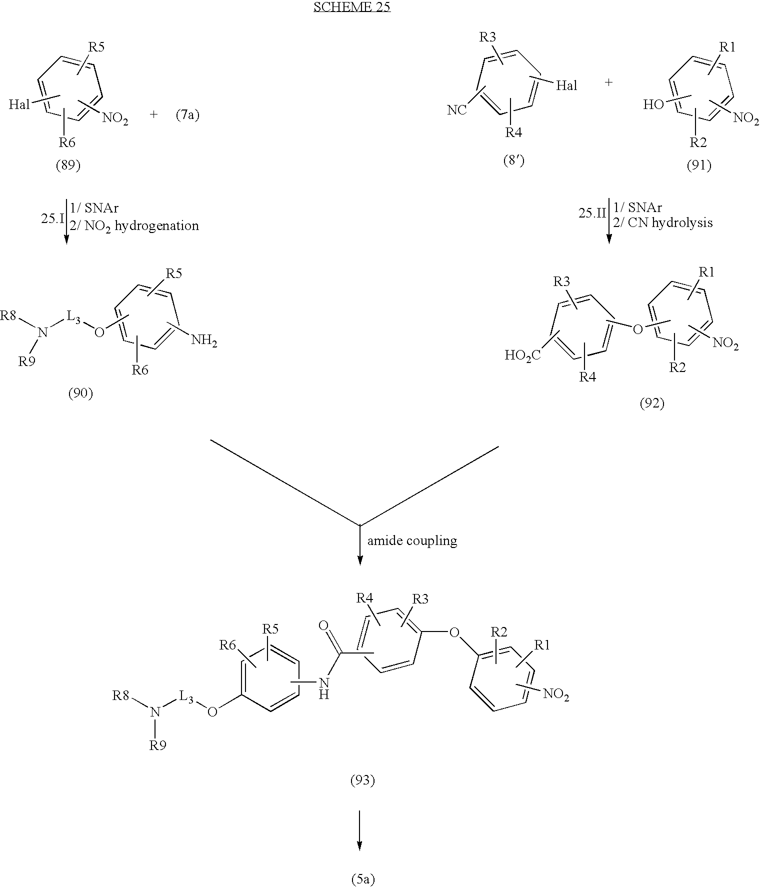

A further particular subject-matter of the invention concerns a method for preparing amines of formula (6a) or (6b),

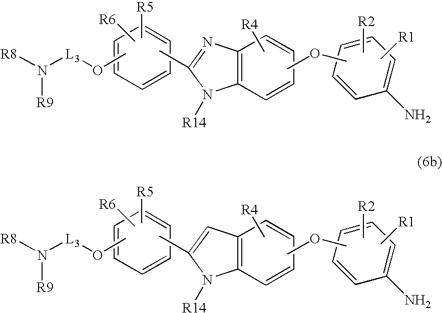

derived from formula (6) in which:
Ar1 and Ar3 are phenyl radicals,
Ar2 is a benzimidazolyl or indolyl radical,
Z′ is a NH₂ radical,
L1 and A are oxygen atoms,
the group:

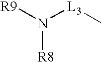

represents:

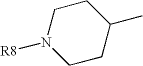

R8, R14 and R1 to R6 are such as defined in formula (I).

The amines (6a) are advantageously prepared as indicated in SCHEME 26 from the key nitroaniline intermediate (96): catalytic hydrogenation of the nitro group of (96) is followed by acylation of the aniline thus obtained by an acid (1a) and cyclization of the intermediate (97) in an acid medium, preferably aqueous HCl, at temperatures lying between 20° C. and 100° C., for 1 h to 24 h, to afford the benzimidazole (98). The methoxy function of (98) is deprotected in an acid medium, preferably concentrated HBr, at temperatures lying between 20° C. and 135° C., for 1 h to 6 h, followed by a SNAr reaction with an aromatic nitrohalogenated derivative (89′) in which Hal represents a chlorine or fluorine atom. Catalytic hydrogenation of the nitro function of (99) produces the aniline (6a). The key intermediate (96) is prepared by N-alkylation of a nitroaniline (94) by an aliphatic halogenated derivative R14-Hal1, Hal1 preferably being a chlorine, bromine or iodine, in an anhydrous solvent such DMF, in the presence of a base such as NaH, at between 0° C. and 30° C., for 24 h to 96 h; or else (96) is obtained by SNAr, with a suitable amine, of the aromatic nitrohalogenated derivative (95) in which Hal2 is a chlorine or fluorine atom. (94) and (95) are commercially available or obtained following procedures known to persons skilled in the art.

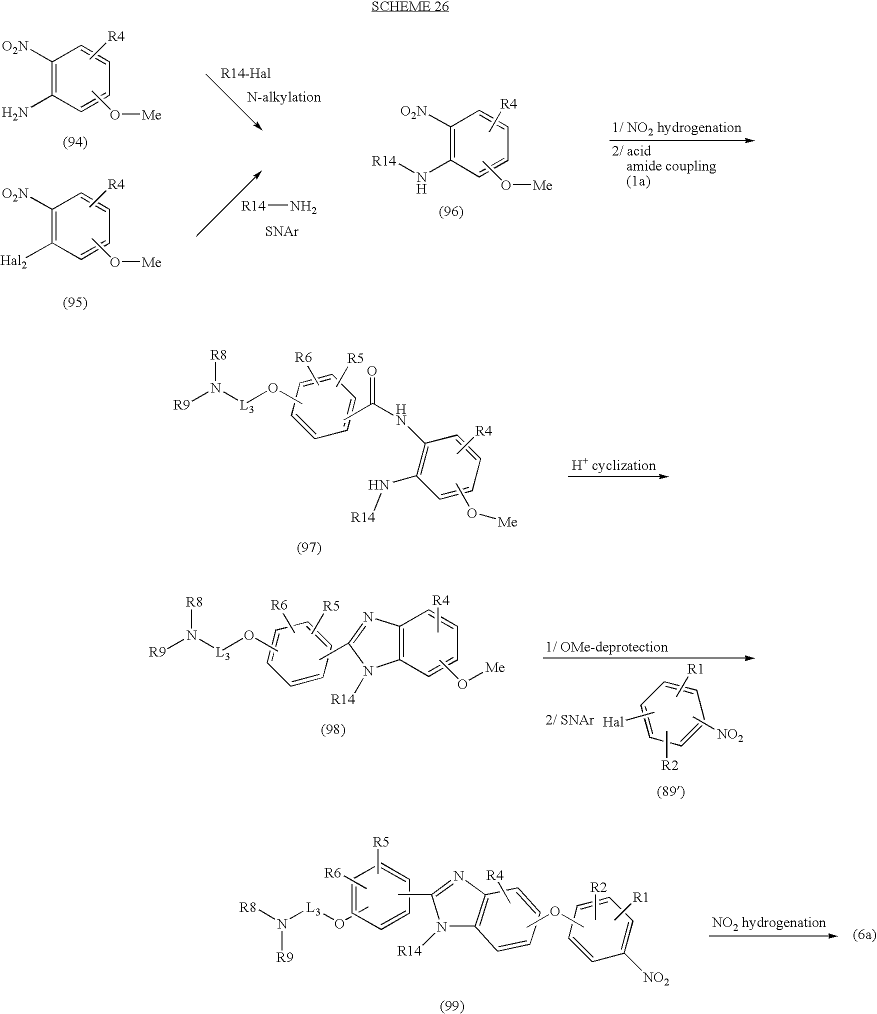

The amines (6b) are advantageously prepared as indicated in SCHEME 27: an aniline of type (100) is acylated by an acid (1a). The intermediate (101) thus obtained is cyclized into the indole in the presence of butyl lithium in an anhydrous solvent such as THF, at temperatures lying between 0° C. and 30° C. for 24 h, followed by alkylation of the indolic NH function with an aliphatic halogenated derivative R14-Hal1 under the conditions described for alkylation of the intermediate (30a), Scheme 9a. The methoxy function of the intermediate (102) thus obtained is deprotected in an acid medium, preferably concentrated HBr, at temperatures lying between 20° C. and 135° C., for 1 h to 6 h, followed by a SNAr reaction with an aromatic nitrohalogenated derivative (89′). Catalytic hydrogenation of the nitro function (103) produces the aniline (6b).

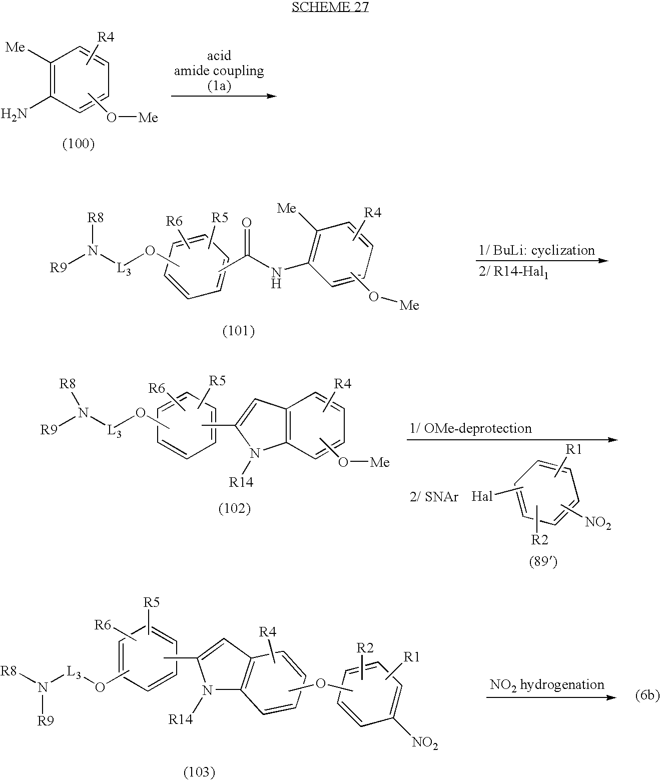

A further particular subject-matter of the invention concerns a method for preparing amines of formula (6c) or (6d),

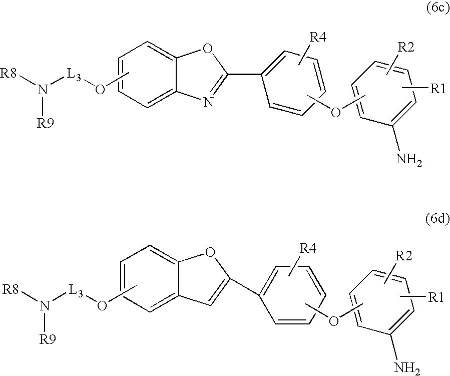

derived from formula (6) in which:
Ar1 and Ar2 are phenyl radicals,
Ar3 is a benzoxazolyl or benzofuranyl radical,
Z′ is a NH₂ radical,
L1 and A are oxygen atoms,
the group:

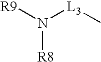

represents:

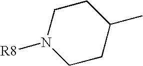

R8 and R1 to R6 are such as defined in formula (I).

The amines (6c) are advantageously prepared as indicated in SCHEME 28: the aminophenol (104) is caused to react with the acid chloride (105), which also used as solvent of the reaction medium, at temperatures lying between 100° C. and 200° C., for 2 h to 24 h, to produce the benzoic benzoxazole ester (106). The ester (106) is saponified and the phenol thus released reacts according to a Mitsunobu reaction with an amino alcohol (7a), in the presence of triphenylphosphine and DIAD in an anhydrous solvent such as THF, at temperatures lying between −20° C. and 30° C., for 24 h to 48 h. The methoxy group of derivative (107) thus obtained is deprotected by the pyridine hydrochloride at temperatures of 160° C. to 190° C. for 1 h to 15 h, followed by a SNAr reaction with an aromatic nitrohalogenated derivative (89′). Catalytic hydrogenation of the nitro function of (108) produces the aniline (6c).

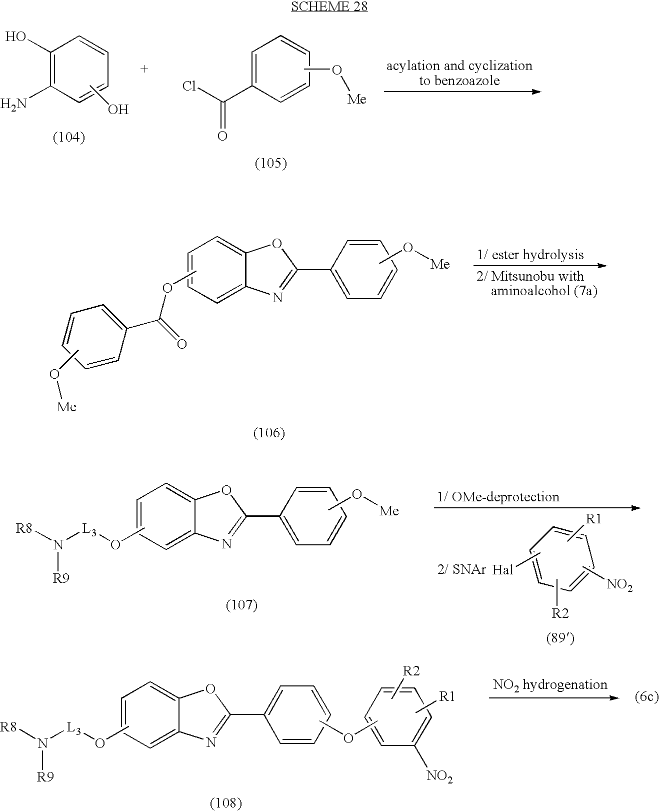

The amines (6d) are advantageously prepared as indicated in SCHEME 29 from the key benzofurane intermediate (109), commercially available or prepared following procedures described in the literature (see René et al Bull. Soc. Chim. Fr., 1973, p 2355-2356). The methoxy group of (109) is deprotected by the pyridine hydrochloride at temperatures of 160° C. to 190° C. for 1 h to 15 h, and the phenol thus released is protected in the form of a silylated ether by reaction of tertbutyldimethyl silyl chloride in the presence of imidazole and DMAP in catalytic quantity in a solvent such as DMF, at AT for 15 h to 24 h. A phenylmethoxy group is then added at position 2- of the benzofurane by the reaction of the silylated derivative (110) with the aromatic iodized derivative (111) in the presence of butyl lithium, zinc bromide and tetrakis triphenylphosphine palladium in an anhydrous solvent such as THF at temperatures lying between −10° C. and 30° C. for 15 h to 24 h. The silylated ether of (112) is deprotected with TBAF in a solvent such as THF, at AT for 3 h to 24 h, and the phenol thus released reacts under a Mitsunobu reaction with an amino alcohol (7a). The methoxy group of the derivative (113) is deprotected with pyridine hydrochloride such as described for (109), followed by a SNAr reaction with an aromatic nitrohalogenated derivative (89′) to obtain the nitro intermediate (114). Catalytic hydrogenation of the nitro function of (114) produces the aniline (6d).

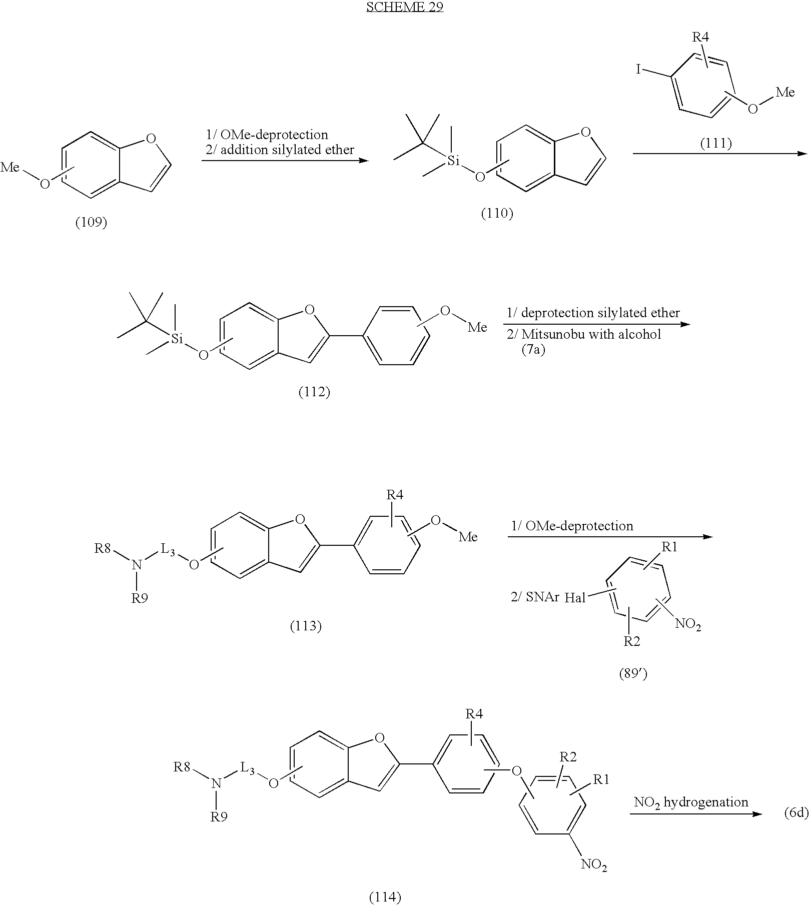

The compounds of the invention fix themselves onto the biological receptors of neuropeptide Y, (NPY), a peptide of 36 aminoacids having multiple physiological activities, in particular in the central nervous or cardiovascular system. NPY controls psychomotor activity, anxiety, sedation, it is a stimulant of food intake; it is involved in depression, memorizing processes, some sexual behaviour and epilepsy; it inhibits the secretion of insulin, of glucagon and the lutinizing hormone; it acts at kidney level and in particular on the renin-angiotensin system; finally it is a powerful vasoconstrictor.

Therefore, the compounds of the invention are advantageously NPY antagonists, preferably of the NPY Y1 receptor. Their IC₅₀, is generally as determined below, 500 nM or less, preferably 100 nM or less, advantageously 50 nM or less, and further advantageously 10 nM or less, even 5 nM or less. More particularly, they are specific antagonists of the NPY Y1 receptor, especially in comparison with other sub-types of NPY receptors, and more specifically by comparison with NPY Y2, Y4 and/or Y5 receptors. Therefore, advantageously, the compounds of the invention have an IC₅₀ for the NPY Y1 receptor that is 10 times lower, preferably 100 times lower, than for the other sub-types of neuropeptide Y receptors, and more specifically by comparison with the NPY Y2, Y4 and/or Y5 receptors.

The compounds of the invention are of particular interest and can be used for the treatment of NPY-dependent pathologies or disorders, advantageously for the treatment of obesity or the treatment of abnormal eating behaviour, or to control food intake, in particular in cases of boulimia or excess fat, on account of their lipolytic activity. They can also be used for the treatment of Type II diabetes and metabolic syndrome. They can additionally be used as antihypertensive agents or for the treatment of vascular diseases, Raynaud's disease, pheochromocytoma, or angina, in particular on account of their vasodilating activity, or to combat coronary and cerebral vasospasm, and for the treatment of athersclerosis and heart failure. They can also be used to treat ischaemia, in particular as neuroprotectors. These compounds may also be useful as anorexigenic agents, antidepressants, tranquillizers, to reduce anxiety or to regulate some sexual behaviour disorders. They are also of true interest for the treatment of pain, inflammation, allergy, some gastro-intetsinal disorders such as Irritable Bowel Syndrome (IBS), Inflammatory Bowel Disease (IBD), or Crohn's disease; they are also immunomodulators. They can further be used to treat problems of drug or alcohol addiction or dependence. Finally, they can be used to regulate the onset of puberty.

According to one aspect of the invention, the above-defined compounds can therefore be used as medicinal products.

A further subject-matter of the present invention is any pharmaceutical composition containing at least one compound such as afore-defined. It is advantageously a pharmaceutical composition for the treatment or prophylaxis of diseases in which neuropeptide Y is involved, and in particular diseases in which the activity of neuropeptide Y is high. The pharmaceutical compositions of the invention can be used in particular for the treatment of obesity, to treat abnormal eating behaviour or to control food intake, in particular in cases of boulimia, or to treat excess fat. They can also be used to treat Type II diabetes and metabolic syndrome. They can also be used for the treatment of hypertension or for the treatment of vascular diseases, Raynaud's disease, pheochromocytoma, or angina, in particular on account of the vasodilating activity of the compounds of the invention, or to combat coronary or cerebral vasospasms, and for the treatment of atherosclerosis and heart failure. They can also be used to treat ischaemia, in particular as neuroprotectors. The pharmaceutical compositions of the invention can additionally be used to treat depression, anxiety or anorexia, or to treat or regulate certain sexual behaviour disorders, to treat pain, inflammation, allergy, or certain gastrointestinal disorders, such as Irritable Bowel Syndrome (IBS), Inflammatory Bowel Disease (IBD), or Crohn's disease. They can also be used to treat drug or alcohol dependence or addiction. Finally, they can be used to regulate the onset of puberty and to treat sexual dysfunctions.

The invention also concerns the use of a compound such as afore-defined for the preparation of a pharmaceutical composition intended to be used to implement a treatment or prophylaxix method for the human or animal body, in particular for the above-mentioned pathologies and disorders.

The invention also concerns a method for treating a pathology in which neuropeptide Y is involved, and in particular the pathologies and disorders mentioned above, comprising the administering of an efficient dose of at least one compound or one pharmaceutical composition such as defined above, to a human patient in particular.

Within the context of the invention, the term <<treatment>> designates preventive, curative, palliative treatments and the management of patients (to reduce suffering, to improve living conditions, to slow progress of the disease) etc. The treatment may also be given in combination with other agents or treatments.

The pharmaceutical compositions of the invention advantageously contain one or more supports, excipients or vehicles that are pharmaceutically acceptable. As examples, mention may be made of saline, physiological, isotonic, buffered solutions, etc. compatible with pharmaceutical use and known to persons skilled in the art. The compositions may contain one or more agents or vehicles chosen from among dispersants, solubilisers, stabilisers, preserving agents, etc. Agents or vehicles which can be used in formulations (liquids and/or injectables and/or solids) particularly include methylcellulose, hydroxymethylcellulose, carboxymethylcellulose, polysorbate 80, mannitol, gelatine, lactose, plant oils, acacia, etc. The compositions can be formulated in the form of injectable suspensions, gels, oils, tablets, suppositories, powders, capsules, etc., optionally using galenic forms or systems ensuring extended and/or delayed release. For this type of formulation, advantageously an agent is used such as cellulose, carbonates or starches.

The compounds or compositions of the invention can be administered in different manners and in different forms. For example they can be administered by parenteral, oral, rectal or nasal route. The parenteral route particularly includes the intravenous, intra-muscular, sub-cutaneous, trans-dermal, and intra-arterial routes. They can also be administered topically, in particular they can be applied to the skin or its appendages. For injections, the compounds are generally packaged in liquid suspension form, which can be injected using syringes or drips for example.

Evidently the flow rate, administered quantity and/or dose can be adapted by those skilled in the art in relation to each patient, pathology, administering method etc.; the compounds are given in daily doses possibly varying between approximately 10 mg and 1000 mg, the dose to be given depending on administering mode and patient weight. Typically, to obtain the desired effect, the dose of active ingredient may vary between 0.1 μg and 100 mg, more specifically between 0.01 and 50 mg per kg body weight per day. Each unit dose may contain 0.5 to 1000 mg, preferably 1 to 500 mg of active ingredients in combination with a pharmaceutical support. This unit dose can be given 1 to 5 times per day so that a daily dose of 0.5 to 5000 mg is received, preferably 1 to 2500 mg.

When preparing a solid composition in tablet form, the main active ingredient is mixed with a pharmaceutical vehicle such as gelatine, starch, lactose, magnesium stearate, talc, gum arabica or similar. The tablets can be coated with sucrose, a cellulose derivative or other suitable matter, or they may be treated so that they have an extended or delayed effect continuously releasing a predetermined quantity of active ingredient.

A capsule preparation is obtained by mixing the active ingredient with a dilutent and by pouring the mixture obtained into soft or hard capsules.

A preparation in syrup or elixir form or for administering in drop form may contain the active ingredient together with a sweetener preferably an acaloric sweetener, methylparaben and propylparaben as antiseptic, and a suitable taste and colouring agent.

Water-dispersible powders or granules may contain the active ingredient in a mixture with dispersing or wetting agents, or suspending agents such as polyvinylpyrrolidone, or with sweeteners and taste correctors.

For rectal administering, suppositories are used prepared with binders which melt at rectal temperature e.g. cocoa butter or polyethyleneglycols.

For parenteral administering, aqueous suspensions, isotonic saline solutions or sterile, injectable solutions are used which contain dispersing agents and/or wetting agents that are pharmacologically compatible e.g. propyleneglycol or butyleneglycol.

The active ingredient can also be formulated in microcapsule form, optionally with one or more supports or additives.

The compositions of the present invention may, in addition to the compounds of the invention, contain other active ingredients which can be used to treat the above diseases or disorders.

### FIGURES

FIG. 1: Effects on arterial hypertension induced by [Leu³¹, Pro³⁴] NPY in anaesthetized rats: compound of example 312 administered orally at 3 mg/kg.

Other aspects and advantages of the present invention will become apparent on reading the following examples which are to be considered as illustrative and non-limiting.

### Materials and Methods

HPLC/MS analyses, unless otherwise specified, were performed on a Waters Micromass ZQ 2000 spectrometer using a XTerra® MS C18 3.5 μm column, 2.1×30 mm, for separation, and for elution using a binary gradient of 100% solvent A to 100% solvent B in 2 min, with a plateau of 1 min at 100% solvant B, the flow rate being 1 ml/min, solvent A being a water/0.05% TFA mixture and solvent B being an ACN/water/TFA mixture (80:20:0.05 v/v/v). Detection of the molecular ion of the products was made using the APCI⁺ or ESI⁺ technique.

Purifications by semi-preparative HPLC in TFA medium were conducted on a Shimadzu line using for separation an Uptisphere 50 DB 100×28 mm column at a flow rate of 50 ml/min for quantities of more than 100 mg of product to be purified, and a YMC-pack ODSA 100×20 mm column at a flow rate of 20 ml/min for quantities of less than 100 mg of product, elution being performed using a binary gradient of solvent A (water/0.05% TFA) and solvent B (ACN/water/TFA 80:20:0.05 v/v/v).

Purifications by semi-preparative HPLC in ammonium bicarbonate medium were performed on a Waters Micromass ZQ 2000 spectrometer using as separating column a XTerra® Prep MS C18 3 μm, 30×50 mm column, and for elution a binary gradient of solvent A (10 mM aqueous solution of ammonium bicarbonate pH 9.5) and solvent B (ACN).

Nuclear magnetic resonance spectra were obtained in deuterated DMSO unless otherwise specified, using Brucker apparatus at 400 MHz and chemical shifts are expressed in ppm. The abbreviations used below are the following: s=singlet; d=doublet; t=triplet; m=multiplet.

Elemental organic analysis was conducted by combustion at 1000° C. in the presence of oxygen, using a scale of UM3 Mettler type and an elemental analyzer of EA 1110 type. Centesimal analyses of the carbon, hydrogen, nitrogen and sulfur elements tally with expected theoretical results.

Unless otherwise specified, the different intermediates used for synthesizing the preparations and compounds of formula (I) are commercially available and were used without any preliminary purifications, or were prepared following protocols well known to persons skilled in the art. The experimental protocols given below are in no way limiting and are given for illustration purposes only.

### General Procedures

### General Procedure A: Saponification of Esters to Carboxylic Acids

The ester is placed in solution or suspension in an ethanol/water medium (1:12 v/v), heated under reflux 3 h in the presence of potassium carbonate de potassium and the ethanol is evaporated in vacuo. If an amino acid is obtained, neutralization is achieved by bubbling sulfur dioxide. The desired product is precipitated and isolated by filtering, or it is extracted in a solvent such as DCM, TBME or ethyl acetate. In this latter case, the organic solvent is dried over MgSO₄, filtered and the desired product is precipitated in hydrochloride form by treatment with a concentrated HCl solution. Unless otherwise specified, the product is used as such.

### General Procedure B: Hydrolysis of the Nitrites to Carboxylic Acids

B1/ hydrolysis of the nitrites in a basic medium: the nitrile is placed in solution in an ethanol/water or methoxyethanol/water mixture (1:2 v/v) and heated under reflux in the presence of KOH or NaOH (5 eq). The progress of the reaction is followed by HPLC until full conversion of the nitrile, which is then concentrated in vacuo, the residue, is redissolved in water and neutralized by bubbling sulfur dioxide. The formed precipitate is filtered, rinsed with water and then with TBME or acetone. In some cases, the product is redissolved in a solvent such as diethyl ether, diisopropyl ether or isopropanol and it is precipitated in hydrochloride form by treatment with a concentrated HCl solution. Unless otherwise specified, the product is used as such.

B2/ hydrolysis of the nitrites in an acid medium: the nitrile is placed in solution in a water/HCl mixture (1:1 v/v) and heated under reflux. The progress of the reaction is followed by HPLC until full conversion of the nitrile. After cooling, the precipitate is filtered, washed with water and oven dried. The product is used as such.

General Procedure C: Deprotection of the BOC Amines with Trifluoroacetic Acid

The amine protected by a BOC group is placed in solution in DCM, and TFA is added (700 ml/mmol) at 0° C. and stirred for 1 h to 12 at AT. The amine is obtained in TFA salt form after evaporating the reaction medium in vacuo and precipitation with diethyl ether or pentane. If the residue is oily, it is redissolved in water and the desired product is precipitated in free base form by placing in a basic medium with aqeuous ammonia. Unless otherwise specified, the product is used as such.

### General Procedure D: Debenzylation of the Amines by Catalytic Hydrogenation

The amine is placed in solution in an ethyl acetate/acetic acid solution (10:1 v/v), and the reaction medium is subjected to catalytic hydrogenation at AP and at AT for 3 h to 5 h in the presence of 10% palladium on charcoal. The desired product is obtained after filtering the catalyst and rinsing with ethyl acetate, followed by evaporation of the filtrate to dryness. Unless otherwise specified, the product is used as such.

### General Procedure E: Reduction of the Nitro-Groups by Catalytic Hydrogenation

The nitro derivative in solution in THF, ethyl acetate or methanol (20 ml/mmol) is treated with hydrogen in the presence of a catalytic quantity of Raney Nickel at AP and AT. The desired product is obtained by filtering the catalyst and rinsing with the reaction solvents, followed by evaporation of the filtrate to dryness. Unless otherwise specified, the product is used as such.

### General Procedure F: Protection of the Amine Functions by a Tertbutyl Carbamate (BOC)

To a solution of amine in THF (0.7 ml/mmol) is added at 0° C. a solution of BOC₂O (1.1 eq) in THF (0.3 ml/mmol) and stirred for 2 h to 24 at AT. The reaction medium is concentrated to dryness, the residue is redissolved in DCM or ethyl acetate, washed with an aqueous 1N solution of HCl, then with an aqueous solution of sodium bicarbonate. The organic layer is dried over MgSO₄, filtered and evaporated in vacuo. Unless otherwise specified, the product is used as such.

General Procedure G: Synthesis of Imidazole-1-carboxylic Acid (1-ethyl-propyl) Amide

To a solution of 1-ethyl-propylamine in THF (10 ml/g amine) cooled to −5° C., is added 1 eq of CDI is added and stirred 15 h at AT. The solvent is evaporated in vacuo, the residue is redissolved in water, extracted with DCM, the organic layer is washed with water, dried over MgSO4, filtered and evaporated in vacuo. The residue obtained is redissolved in pentane, the supernatant is removed and the residue is again concentrated in vacuo. The desired product is obtained in the form of thick oil.

General Procedure H: Synthesis of Ureas Using Imidazole-1-carboxylic Acid (1-ethyl-propyl)-amide

The amine is placed in solution in THF or acetonitrile (25 ml/mmol), and 2 to 5 eq of imidazole-1-carboxylic acid (1-ethyl-propyl)-amide are added and DIEA to neutralize the salts if the amine is salified. The mixture is heated under reflux for 48 h to 168 h, concentrated in vacuo, the residue is redissolved in water, extracted with TBME or DCM, the organic layer is dried over MgSO₄, filtered and evaporated in vacuo. The desired product is isolated after precipitation with a solution of HCl in diethyl ether or after purification by chromatography on silica, or semi-preparative HPLC.

### General Procedure I: Synthesis of Amides in the Presence of TBTU/HOBT

The carboxylic acid is solubilized in a 0.4M mixture of TBTU/HOBT in DMF, with 1.1 eq to 1.3 eq of each reagent relative to the acid, then 3.2 eq to 3.6 eq of DIEA are added and the reaction medium is stirred at AT for 5 min to 1 h. The addition is made of 1 eq of amine and the quantity of DIEA necessary to neutralize the salts if the amine is salified, the medium is left under stirring for 2 h to 96 h at AT or 60° C., then the solvent is evaporated in vacuo. The desired product is isolated after purification by semi-preparative HPLC or chromatography on silica.

### General Procedure J: Synthesis of Amides in the Presence of TBTU

The carboxylic acid, 1 eq TBTU, 1 eq amine and 2 eq TEA are placed in solution in DMF (5 ml/0.3 mmol), and stirred for 15 h at AT, then the solvent is evaporated in vacuo. The desired product is isolated after purification by semi-preparative HPLC or by chromatography on silica.

### General Procedure K: Synthesis of Amides in the Presence of PyClu

The carboxylic acid, 1 eq amine, 1 eq PyClu and 3 eq DIEA are placed in suspension in DCM (1 ml/0.1 mmol), and stirred for 10 min at AT, then xylene is added (6 ml/0.1 mmol) and heated under reflux for 2 h. The solvent is evaporated in vacuo and the desired product is isolated after purification by semi-preparative HPLC.

### General Procedure L: Synthesis of Amides in the Presence of EDCI

L1/ The carboxylic acid, 1.2 eq HOBT, 1.2 eq EDCI and 2 eq to 4 eq DIEA are placed in solution in DMF (3 ml á 10 ml/1 mmol), stirred at AT for 30 min to 2 h, 1 eq of amine solubilized in DMF (2 ml to 5 ml/1 mmol amine) is added and the reaction medium is stirred for 24 h to 72 h at AT. The solvent is evaporated in vacuo, the residue is redissolved in water, the precipitate obtained is filtered and washed with an aqueous sodium bicarbonate solution and with water. The desired product is isolated after purification of this precipitate by semi-preparative HPLC or chromatography on silica.

L2/ The operating mode described in General Procedure L1 is used, coupling of the amine being conducted 16 h at AT, followed by 4 h at 60° C.

L3/ The operating mode described in General Procedure L1 is followed, but without any prior activation of the acid, the amine being added to the reaction medium at the same time as the acid.

L4/ The operating mode described in General Procedure L3 is followed, coupling of the amine being conducted 6 h at 60° C. followed by 16 h at AT.

General Procedure M: Synthesis of Amides in the presence of TOTU

The carboxylic acid is activated in the presence of 1.2 eq TOTU and 2 to 5 eq DIEA in DCM (10 to 30 ml/1 mmol) at AT for 15 min to 30 min. 1 eq of amine is then added solubilized in a minimum quantity of DMF and stirred for 15 h at AT. The solvent is evaporated in vacuo and the desired product is isolated after purification by semi-preparative HPLC or chromatography on silica.

General Procedure N: Synthesis of Urea Using a Suitable Isocyanate

The amine is placed in solution in THF (12 ml/1 mmol) in the presence of a catalytic quantity of pyridine and DIEA to neutralize salts if the amine is salified, 1.1 eq isocyanate is added and heated under reflux 4 h to 12 h. The reaction medium is concentrated, the residue redissolved in diisopropyl ether, the precipitate obtained is filtered and rinsed with diisopropyl ether and with pentane. The desired product is obtained which is used as such, or after purification by semi-preparative HPLC or chromatography on silica.

General Procedure O: Condensation of a Phenol on a 4-fluoronitrobenzene or 4-chloronitrobenzene

To a suspension of NaH (1.3 eq) in DMF, the phenol (1.2 eq) is added dropwise, heated at between 50° C. and 80° C. for 45 min to 2 h, then the nitrohalogenated derivative (1 eq) in solution in a minimum quantity of DMF is rapidly added dropwise, heated again between 90° C. and 150° C. for 3 h to 48 h. After concentration in vacuo, the residue is redissolved in water, extracted with ethyl acetate, the organic layer is washed with an aqueous NaOH solution, with an aqueous NaCl solution, and the organic layer is dried over MgSO₄, filtered and evaporated in vacuo. The desired product is obtained which is used as such, or after purification by chromatography on silica.

General Procedure P1: Condensation of a Nitrophenol on 2-amino-5-Bromothiazole.

To a suspension of NaH (2.1 eq) in DMF (1.3 ml/1 mmol) is added the nitrophenol (2 eq) in solution in DMF (1.3 ml/1 mmol), heated 1 h at 60° C., then 2-amino-5-bromothiazole (1 eq) in solution in DMF (1.3 ml/1 mmol) is added dropwise and left under stirring 15 h at AT. After concentration in vacuo, the residue is redissolved in water, extracted with diethyl ether, and the black tar is removed by filtering. The organic layer of the filtrate is washed with an aqueous NaOH solution, and the organic layer is dried over MgSO₄, filtered and evaporated in vacuo. The desired product is obtained, which is used as such or after purification by chromatography on silica.

General Procedure P2: Condensation of a Nitrophenol on 2-amino-5-bromothiazole.

To a solution of 2-amino-5-bromothiazole in a minimum quantity of ethanol, heated to around 60° C., is added a mixture of K₂CO₃ (1 eq)/nitrophenol (1 eq) in water/ethanol (1:2 v/v) and heated under reflux for 1 h. The tars are filtered and evaporation conducted in vacuo. The residue is redissolved in DCM, the precipitate formed is filtered, the filtrate is washed with an aqueous NaOH solution, dried over MgSO₄, filtered and evaporated in vacuo. The product is isolated after purification by chromatography on silica.

### Preparations

### Preparation 1

### 4-(3-piperidin-1-yl-propoxy)-benzoic acid

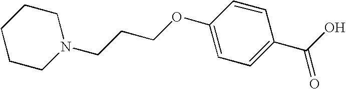

### A/ 4-(3-Chloro-propoxy)methyl benzoate

To a suspension of 30 g of Methyl-4-hydroxybenzoate and K₂CO₃ (2 eq) in acetone (400 ml), is added 1-bromo-3-chloropropane (1.5 eq) dropwise, then heated under reflux 12 h, filtered, and the filtrate evaporated to dryness. 45 g of desired product are obtained which is used as such.

### B/ 4-(3-Piperidin-1-yl-propoxy)methyl benzoate

In the presence of piperidine (3.05 eq), 6 g of the compound obtained in the previous step are heated during 16 h in solution in MeOH (50 ml). After concentration, the residue is redissolved in DCM, washed with water and with a saturated aqueous NaHCO₃ solution, the organic layer is dried over MgSO₄, filtered and concentrated to dryness. 6.7 g of the desired product are isolated, which is used as such.

### C/ 4-(3-piperidin-1-yl-propoxy)-benzoic acid

Following General Procedure A, 2.85 g of the desired product are obtained from the compound of the preceding step.

### Preparation 2

### 4-(1-Isopropyl-piperidin-4-yloxy)-benzoic acid

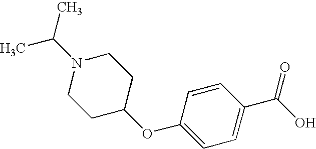

### A/ 1-Isopropyl-piperidinol

To a suspension of 4-hydroxypiperidine (30 g) and Na₂SO₄ (20 g) in 600 ml of chloroform, is added 24 ml of acetone and stirred for 24 h at AT, then 120 g of sodium triacetoxyborohydride are gradually added, and stirred for a further 24 h at AT. 400 ml MeOH are added dropwise, stirring continued for 2 h at AT and the solvent evaporated in vacuo. The residue is redissolved in 40 ml water, basified, extracted with DCM and the organic layer is evaporated in vacuo. 24.1 g of the desired product are obtained.

### B/ 4-(1-Isopropyl-piperidin-4-yloxy)-benzonitrile

The compound prepared in the preceding step is solubilized in DMF, 8 g NaH is added and stirred at AT for 1 h, 20.6 g of 4-fluorobenzonitrile are added and stirring continued for 4 h at AT. After evaporation to dryness, the residue is redissolved in water, extracted with TBME, the organic phase is extracted with acid water (HCl), this aqueous phase is basified and the product is extracted with TBME. The final organic layer is dried over MgSO₄ and evaporated in vacuo. 35.5 g of the desired product are obtained.

### C/ 4-(1-Isopropyl-piperidin-4-yloxy)-benzoic acid

The desired product is obtained from the compound of the preceding step by base hydrolysis, following General Procedure B.

### Preparation 3

### 4-(1-Butyl-piperidin-4-yloxy)-3-methoxy-benzoic acid

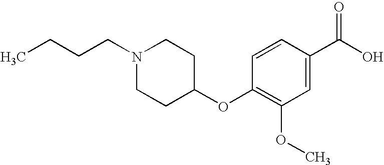

### A/ 1-Butyl-piperidin-4-ol

A mixture of 20 g of 4-hydroxypiperidine, of butyraldehyde (1 eq) and of Na₂SO₄ (4.7 eq) in chloroform (400 ml) is stirred at AT for 24 h, sodium triacetoxyborohydride (3 eq) is added and stirring is continued at AT for 24 h. Then 267 ml MeOH are added dropwise and the mixture stirred for 2 h at AT. After evaporation to dryness, the residue is redissolved in base water, extracted with TBME, the organic layer is dried over MgSO₄, filtered and evaporated. 26 g of the desired product are obtained.

### B/ 4-(1-Butyl-piperidin-4-yloxy)-3-methoxy-benzonitrile

A suspension of NaH (1.25 eq) in DMF (100 ml), to which is added 10 g of the product obtained during the preceding step, is stirred for 1 h at AT, then 4-fluoro-3-methoxy-benzonitrile (1 eq) in DMF (100 ml) is added and stirred a further 24 h at AT. After evaporation to dryness, the residue is redissolved in water, extracted with TBME, the organic layer is washed with a 1 N aqueous solution of NaOH then with an aqueous NaCl solution, dried over MgSO₄, filtered and evaporated. 10.1 g of the desired product are isolated after chromatography on silica eluting with a DCM/MeOH/NH₄OH mixture (97.5:2.5:0.1 v/v/v).

### C/ 4-(1-Butyl-piperidin-4-yloxy)-3-methoxy-benzoic acid

12 g of the desired product are obtained from the compound of the preceding step, following General Procedure B.

### Preparation 4

### 4-(1-Butyl-piperidin-4-yloxy)-3-methyl-benzoic acid

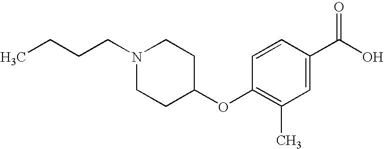

### A/ 4-(1-Butyl-piperidin-4-yloxy)-3-methyl-benzonitrile

To a suspension of NaH (1.95 eq) in DMF (20 ml) are added 5.2 g of 1-butyl-piperidin-4-ol (Preparation 3, step A), the mixture is heated 3 h at 60° C., then 4-chloro-3-methyl-benzonitrile (1 eq) in DMF (20 ml) is added and heating continued for a further 4 h at 90° C. After evaporation to dryness, the residue is redissolved in water, extracted with TBME, the organic layer is washed with a 1 N aqueous solution of NaOH then with an aqueous NaCl solution, dried over MgSO₄, filtered and evaporated. 4.5 g of the desired product are isolated after chromatography on silica eluting with a DCM/MeOH/NH₄OH mixture (95:5:0.5 v/v/v).

### B/ 4-(1-Butyl-piperidin-4-yloxy)-3-methyl-benzoic acid

4.2 g of the desired product are obtained from the compound of the preceding step by base hydrolysis, following General Procedure B.

### Preparation 5

### 4-(1-Butyl-piperidin-4-yloxy)-benzoic acid

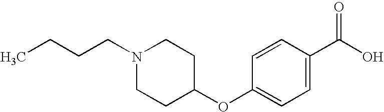

### A/ 4-(1-Butyl-piperidin-4-yloxy)-benzonitrile

A suspension of NaH (1.95 eq) in DMF (100 ml), to which 26 g of 1-butyl-piperidinol-4-ol (Preparation 3, step A) are added, is stirred 3 h at AT, then 1 eq of 4-fluorobenzonitrile in DMF (100 ml) is added and the medium stirred a further 24 h at AT, and the solvent evaporated to dryness. The residue is redissolved in water, extracted with TBME, the organic layer is washed with an aqueous 1 N NaOH solution, then with an aqueous NaCl solution, dried over MgSO₄, filtered and evaporated. 13.7 g of the desired product are isolated after chromatography on silica eluting with a DCM/MeOH/NH₄OH mixture (98:2:0.1 v/v/v).

### B/ 4-(1-Butyl-piperidin-4-yloxy)-benzoic acid

9 g of the desired product are obtained from the compound of the preceding step by base hydrolysis, following General Procedure B.

### Preparation 6

### 4-(1-Methyl-piperidin-4-yloxy)-benzoic acid

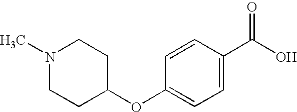

### A/ 4-(1-Methyl-piperidin-4-yloxy)-benzonitrile

A mixture of N-methyl-4-hydroxypiperidine (6.28 g), NaH (0.9 eq) and 4-fluorobenzonitrile (0.9 eq) in 100 ml of DMF is stirred at AT for 24 h, then evaporated dry. The reaction medium is redissolved in water, extracted with ethyl acetate, the organic layer is dried over MgSO₄, filtered and concentrated. 5.5 g of desired product are obtained after redissolving this residue in pentane, filtering and drying the precipitate.

### B/ 4-(1-Methyl-piperidin-4-yloxy)-benzoic acid

5.5 g of desired product are obtained from the compound of the preceding step, by base hydrolysis following General Procedure B.

### Preparation 7

### [(4-cis)-4-Carboxy-phénoxy)cyclohexyl]-trimethyl-ammonium

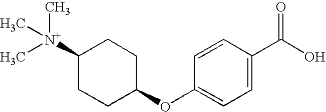

### A/ 4-[(4-cis)-(1,3-Dioxo-1,3-dihydro-isoindol-2-yl)cyclohexyloxy]-methyl benzoate

In an inert atmosphere, 20 g of 2-trans-4-hydroxy-cyclohexyl)isoindole-1,3-dione are added to a mixture of methyl hydroxybenzoate (1 eq) and triphenylphosphine (2.1 eq) in THF (160 ml), and stirred 15 min at AT, then DIAD (2.1 eq) is added slowly keeping the temperature to below 45° C., and the mixture stirred a further 48 h at AT, the solvent is then evaporated in vacuo. 12.9 g of the desired product are obtained in the form of a pink powder, after chromatography on silica eluting with DCM.

### B/ 4-[(4-cis-Amino-cyclohexyloxy]-methyl benzoate

15.3 g of product obtained such as described in the preceding step are heated under reflux for 3 h in the presence of hydrazine hydrate (5 eq) in ethanol (700 ml). After evaporation in vacuo, the residue is redissolved in an aqueous 1 N solution of HCl, the insoluble is filtered, the filtrate is basified, extracted with TBME, the organic layer is dried on MgSO₄, filtered and evaporated. 5.9 g of the desired product are obtained in oil form.

### C/ [(4-cis)-(4-Methoxycarbonyl-phenoxy)-cyclohexyl]-trimethyl-ammonium

A solution of 500 mg of the compound obtained in the previous step in THF (5 ml) is heated at 35° C. for 2 h in the presence of K₂CO₃ (3 eq) and methyl iodide (3 eq). After evaporation in vacuo, the residue is redissolved in water, extracted with DCM, the organic layer is dried over MgSO₄, filtered and evaporated. 600 mg of the desired product are obtained in the form of a white powder.

### D/ [(4-cis)-(4-Carboxy-phenoxy)-cyclohexyl]-trimethyl-ammonium

0.96 g of the desired product are isolated by following General Procedure A to treat 2.1 g of the compound obtained such as described in the preceding step.

### Preparation 8

### 4-(1-Butyl-piperidin-4-yloxy)-2-fluoro-benzoic acid

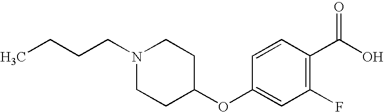

### A/ 4-(1-Butyl-piperidin-4-yloxy)-2-fluoro-benzonitrile

To a suspension of NaH (1 eq) in DMF (25 ml), containing 7.3 g (46 mmol) of 1-butyl-piperidin-4-ol prepared as described under Preparation 3, step A, cooled to 0° C., is added 2,4-difluorobenzonitrile (1.1 eq) and stirred for 15 h at AT. The solvent is evaporated in vacuo, the residue redissolved in water, extracted with TBME, the organic layer is extracted with 1 N aqueous HCl, this aqueous phase is basified and extracted with TBME. This last organic layer is dried over MgSO₄, filtered and concentrated to dryness. A mixture is obtained that is 57% enriched with the desired product after purifying the residue by chromatography on silica eluting with a 98:2 DCM/MeOH mixture. The product is dissolved in acetone, concentrated HCl is added, evaporated to dryness, and recrystallized in ACN. 0.8 g of a mixture is obtained, 87% enriched with the desired product. This product is used as such.

### B/ 4-(1-Butyl-piperidin-4-yloxy)-2-fluoro-benzoic acid

A solution of the compound obtained in the preceding step is heated under reflux for 35 h in a water/concentrated HCl mixture, then concentrated in vacuo. The crystals obtained are filtered, the filtrate is collected and concentrated to dryness. 650 mg of the desired product are obtained.

### Preparation 9

### 4-(1-Butyl-piperidin-4-yloxy)-3-fluoro-benzoic acid

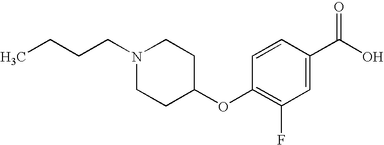

### A/ 4-(1-Butyl-piperidin-4-yloxy)-3-fluoro-benzonitrile

To a solution of NaH (330 mg; 1.3 eq) in DMF (35 ml) is added 1-butyl-piperidinol (1 g; 1 eq) obtained such as described under Preparation 3, step A, and heated at 60° C. for 30 min, a solution of 3,4-difluorobenzonitrile (884 mg; 1 eq) in DMF (10 ml) is added and the mixture heated at 80° C. for a further 15 h. The reaction medium is diluted with water, extracted with ethyl acetate, and the organic layer is washed several times with water, dried over MgSO₄, filtered and concentrated to dryness. 600 mg of the desired product are isolated after chromatography on silica eluting with a cyclohexane/ethyl acetate mixture (50:50 v/v).

### B/ 4-(1-Butyl-piperidin-4-yloxy-3-fluoro-benzoic acid

Following General Procedure B, 650 mg of desired product are isolated by treating 650 mg of the compound obtained such as described in the preceding step.

### Preparation 10

### 4-(1-Butyl-piperidin-4-yloxy)-3-trifluoromethyl-benzoic acid

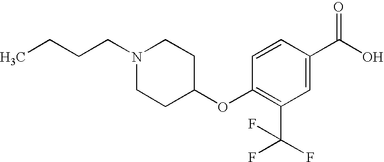

### A/ 4-(1-Butyl-piperidin-4-yloxy)-3-trifluoromethyl-benzonitrile

To a suspension of NaH (1.3 eq) in DMF (5 ml), is added 1 g of 1-butyl-piperidin-4-ol prepared such as described under Preparation 3, step A, in DMF (5 ml), and stirred for 1 h at AT, followed by the addition of a solution of 3-trifluoromethyl-4-fluorobenzonitrile (1 eq) in DMF (5 ml), then stirring is continued at AT for 15 h. Water is added, the medium is extracted with TBME, the organic layer is washed several times with water, dried over MgSO₄, filtered and concentrated to dryness. 1.1 g of the desired product is isolated after chromatography on silica eluting with a DCM/MeOH mixture (90:10 v/v).

### B/ 4-(1-Butyl-piperidin-4-yloxy)-3-trifluoromethyl-benzoic acid

360 mg of the desired product are obtained by treating the compound obtained in the preceding step, following General Procedure B.

### Preparation 11

### 4-(1-Benzyl-piperidin-4-yloxy benzoic acid

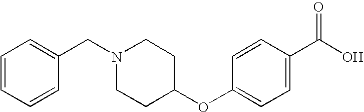

### A/ 4-(1-Benzyl-piperidin-4-yloxy)-benzonitrile

To a solution of NaH (1 eq) in DMF (300 ml), is added 1-benzyl-piperidinol (40 g), and the mixture stirred at AT for 30 min, followed by the addition of 4 fluorobenzonitrile (1 eq) in DMF (100 ml), and continued stirring for a further 24 h at AT, and finally the solvent is evaporated in vacuo. The residue is redissolved in diethyl ether, the organic layer is washed in water, dried over MgSO₄, filtered and evaporated. 58 g of the desired product are isolated.

### B/ 4-(1-Benzyl-piperidin-4-yloxy)-benzoic acid

Following General Procedure B, 16.3 g of the desired product are isolated by treating 20 g of the compound obtained in the preceding step.

### Preparation 12

### 4-(1-Isopropyl-piperidin-4-ylmethoxy)-benzoic acid

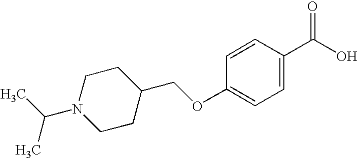

### A/ 4-Methanesulfonyloxymethyl-piperidine-1-tertbutyl carboxylate

Following the procedure described by Waterhouse, J. Labelled. Compd. Radiopharm., 1996, 38 (3) pp 215-226, methane sulfonyl chloride (1.2 eq) is caused to react with 13.6 g of BOC-isonicot(H6)-ol in solution in DCM (190 ml) in the presence of TEA (3.5 eq). 16 g of the desired product are obtained in the form of a white solid.

### B/ 4-(4-Methoxycarbonyl-phenoxymethyl)piperidine-1-tertbutyl carboxylate

To a suspension of NaH (3 eq) in 95 ml DMF, is added methyl-4-hydroxybenzoate (4 eq) and then 7 g of compound obtained in the previous step, and heated 7 h at 60° C. The reaction medium is diluted with diethyl ether, washed with a 30% aqueous NaOH solution, the organic layer is dried over MgSO₄, evaporated in vacuo, and the residue is redissolved in pentane, filtered, and the precipitate obtained is dried. 7.8 g of the desired product are isolated in the form of a white powder.

### D/ 4-Piperidin-4-ylmethoxy)-methyl benzoate

3.6 g of desired product are obtained by deprotecting the BOC group of the compound obtained in the previous step, following General Procedure C.

### E/ 4-(1-Isopropyl-piperidin-4-ylmethoxy)-methyl benzoate

2.6 g of compound obtained in the previous step are reacted with acetone (2 eq) in the presence of sodium triacetoxyborohydride (4 eq), in solution in DCM (21 ml), for 48 h at AT. The reaction medium is then poured into water, basified with an aqueous ammonia solution, the aqueous phase is extracted with DCM, the organic layer is dried over MgSO₄, filtered and evaporated. 1.8 of desired product are obtained in the form of pale yellow crystals.

### F/ 4-(1-Isopropyl-piperidin-4-ylmethoxy)-benzoic acid

The compound obtained in the previous step is heated under reflux for 24 h, in a mixture of MeOH (3 ml)/concentrated HCl (20 ml)/water (20 ml). The reaction medium is concentrated in vacuo, diluted with water, DCM is added, and after filtering the precipitate obtained is rinsed with diethyl ether. 970 mg of desired product are obtained in the form of a white powder.

### Preparation 13

### 4-[(4-cis)-Dimethylamino-cyclohexyloxy]-benzoic acid

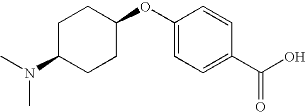

### A/ 4-[(4-cis)-Dimethylamino-cyclohexyloxy]-methylbenzoate

1 g of compound obtained such as described under Preparation 7 step B, in solution in a mixture of formic acid (5.6 eq)/formaldehyde (1 ml of 37% solution in water) is heated under reflux for 24 h. After concentration in vacuo, the residue is redissolved in a 1N aqueous HCl solution, the precipitate is filtered, the filtrate is basified, extracted with ethyl acetate, and the organic layer is dried over MgSO₄, filtered and evaporated. 0.7 g of desired product are isolated after precipitation of the residue in diisopropyl ether.

### B/ 4-[(4-cis)-Dimethylamino-cyclohexyloxy]-benzoic acid

Following General Procedure A, 0.5 g of desired product are obtained from the compound of the previous step.

### Preparation 14

### 4-[3-(4-Hydroxy-piperidin-1-yl)-propoxy]-benzoic acid

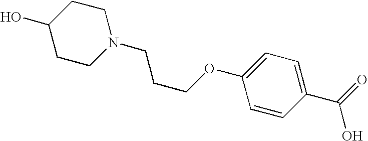

### A/ 4-[3-(4-Hydroxy-piperidin-1-yl)-propoxy]-methyl benzoate

A mixture of 15 g of compound obtained such as described under Preparation 1 step A, and 4-piperidinol (6 eq) is heated under reflux for 10 h in 100 ml of toluene, evaporated, the residue redissolved in DCM, washed with water, and the organic layer is dried over MgSO₄, filtered and evaporated. The desired product is isolated after redissolving this residue in 1 N aqueous HCl, washing with DCM, and evaporating the aqueous layer. This product is used as such.

### B/ 4-[3-(4-Hydroxy-piperidin-1-yl)-propoxy]-benzoic acid

Following General Procedure A, the desired product is isolated in powder form by treating the compound obtained in the previous step.

### Preparation 15

### 4-[1,(cis,cis-2,6)-Trimethyl-piperidin-(cis-4)-yloxy]-benzoic acid

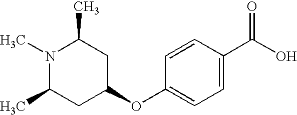

### A/ 1,2,6-Trimethyl-1H-pyridinone

While keeping the temperature below 40° C., 2,6-dimethyl-gamma-pyrone (25 g) in solution in water (72 ml) is added dropwise to a solution of methylamine (54 ml of 40% solution in water) and the reaction medium is mechanically stirred for 2.5 h. It is then cooled down to around 0° C. and the precipitate formed is filtered. 25.1 g of desired product are isolated in the form of a white solid, after recrystallization of the precipitate in water.

### B/ 1,2,6-Trimethyl-piperidin-4-ol

12 g of compound obtained in the previous step in solution in ethanol (160 ml) are hydrogenated in the presence of Raney Ni at a pressure of 120 bars at 125° C. for 4.5 h, the catalyst is filtered and the filtrate concentrated. 7.3 g of desired product are isolated after distilling the residue at 3 mm Hg (boiling T=77° C.).

### C/ 4-[1,(cis,cis-2,6-Trimethyl-piperidin-(cis-4)-yloxy]-benzonitrile

The compound obtained in the previous step (6.75 g) in solution in DMF (30 ml) is added to a suspension of NaH (1.1 eq) in DMF (30 ml), and heated 40 min at 55° C., 4-fluorobenzonitrile (1 eq) is added and heating continued for a further 4 h at 65° C. The reaction medium is concentrated, the residue redissolved in water, extracted with diethyl ether, the organic layer is washed with a saturated aqueous NaCl solution and dried over MgSO₄, filtered and evaporated to dryness. The oily residue obtained is redissolved in a diethyl ether/HCl mixture, concentrated in vacuo, redissolved in hot acetone, hot filtered and the precipitate rinsed with acetone. 5.6 g of desired product (cis) are isolated after recrystallizing the precipitate in isopropanol.

### D/ 4-[1,(cis,cis-2,6-Trimethyl-piperidin-(cis-4)-yloxy]-benzoic acid

The compound obtained in the previous step (5 g; 17.8 mmol), in solution in a water (20 ml)/concentrated HCl (40 ml) mixture, is heated under reflux for 18 h. 5.7 g of desired product are obtained after concentrating the reaction medium and washing the residue obtained in acetone. This product is used as such.

### Preparation 16

### 4-[1,(cis,cis-2,6)-Trimethyl-piperidin-(trans-4)-yloxy]-benzoic acid

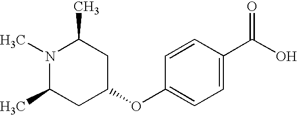

### A/ 4-[1,(cis,cis-2,6-Trimethyl-piperidin-trans-4)-yloxy]-benzonitrile

The reaction mixture obtained such as described under Preparation 38 step C is evaporated in vacuo, the residue is redissolved in water, extracted with diethyl ether, the organic layer is washed with a saturated aqueous NaCl solution, dried over MgSO₄, filtered and evaporated to dryness. The residue is redissolved in a diethyl ether/HCl mixture, concentrated in vacuo, redissolved in hot acetone, hot filtered and the precipitate is rinsed with acetone and the filtrate evaporated to dryness. From this filtrate, 0.43 g of desired product are isolated after chromatography on silica eluting with a DCM/MeOH/NH₄OH mixture (90:10:0.1 v/v/v).

### Preparation 17

### 4-(1-Butyl-piperidin-4-yloxy)-3,5-dimethyl-benzoic acid

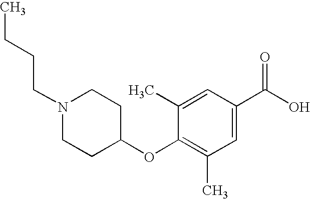

### A/ 4-(1-Butyl-piperidin-4-yloxy)-3,5-dimethyl-benzonitrile

To a mixture of 3,5-dimethyl-4-hydroxy-benzonitrile (4 g), of 1-butyl-piperidinol (3 eq) obtained as described under Preparation 3 step A, and of triphenylphosphine (3 eq) in DCM, is added DIAD (3 eq) dropwise, and stirred 48 h at AT. The reaction medium is washed with water, the organic layer is dried over MgSO₄, filtered and evaporated. This residue is redissolved in diisopropyl ether, the precipitate formed is filtered and the filtrate is collected and evaporated. The desired product is obtained in the form of an orange wax (4.1 g) after chromatography on silica eluting with a DCM/MeOH mixture (95:5 v/v). This product is used as such for the following step.

### B/ 4-(1-Butyl-piperidin-4-yloxy)-3,5-dimethyl-benzoic acid

Following General Procedure B, 0.7 g of desired product are isolated by treating the compound obtained in the previous step.

### Preparation 18

### 4-(1-Butyl-piperidin-4-yloxy)-3-chloro-benzoic acid

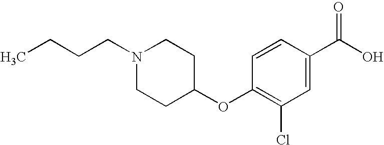

### A/ 4-(1-Butyl-piperidin-4-yloxy)-3-chloro-benzonitrile

To a solution of NaH (1.25 eq) in DMF (100 ml) is added 1-butyl-piperidin-ol (15 g) obtained such as described under Preparation 3 step A, and stirred 1 h at AT, followed by the addition of 3-chloro-4-fluoro-benzonitrile (1 eq) in DMF (100 ml) and continued stirring for a further 24 h at AT. The solvent is evaporated in vacuo, the residue redissolved in water, extracted with DCM, the organic layer is washed with an aqueous 1N NaOH solution then an aqueous NaCl solution, dried over MgSO₄, filtered and evaporated. 20 g of desired product are isolated after chromatography on silica eluting with a DCM/MeOH/NH₄OH mixture (97.5:2.5:0.1 v/v/v).

### B/ 4-(1-Butyl-piperidin-4-yloxy)-3-chloro-benzoic acid

Following General Procedure B, 17 g of desired product are isolated after treating the compound obtained in the previous step.

### Preparation 19

### 4-(1-Isopropyl-piperidin-4-yloxymethyl)-benzoic acid

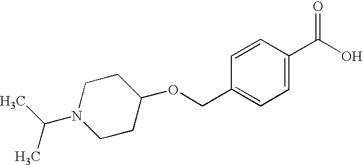

### A/ 4-(4Cyano-benzyloxy)-piperidine-1-tertbutyl carboxylate

To a solution of NaH (1 eq) in DMF (10 ml) is added a solution of 1-BOC-4 piperidinol (5 g), the mixture is stirred 2.5 h at AT, then a 4-cyanobenzyl bromide solution (1.1 eq) in DMF (20 ml) is added and stirring continued for 20 h at AT. The reaction medium is evaporated to dryness, the residue redissolved in water, extracted with ethyl acetate, and the organic layer is washed with an aqueous 1 N NaOH solution, dried over MgSO₄, filtered and evaporated. 5.5 g of desired product are isolated in powder form after chromatography on silica eluting with a 96:4 (v/v) DCM/MeOH mixture.

### B/ 4-(Piperidin-4-yloxymethyl)-benzonitrile

The desired product is isolated following General Procedure C, by treating the compound obtained in the previous step.

### C/ 4-(1-Isopropyl-piperidin-4-yloxymethyl)-benzonitrile

1.4 g of compound obtained in the previous step are reacted with 1.5 eq of acetone in DCM (20 ml) for 30 min., then 3.5 eq of sodium triacetoxyborohydride are added and stirred for 24 h at AT. The reaction medium is washed with an aqueous ammonia solution, dried over MgSO₄, filtered and evaporated. 1.6 g of desired product are obtained in powder form. This product is used as such.

### D/ 4-(1-Isopropyl-piperidin-4-yloxymethyl)-benzoic acid

The desired product is isolated following General Procedure B, by treating the compound obtained in the preceding step.

### Preparation 20

### 4-(1-Isopropyl-piperidin-3-ylmethoxy)-benzoic acid

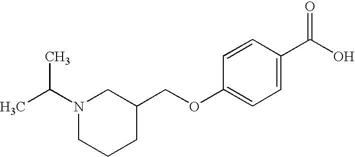

### A/ 3-(4Cyano-phenoxymethyl)piperidine-1-tertbutyl carboxylate

To a suspension of NaH (1.2 eq) in DMF (75 ml) is added 43.8 g of BOC-3-hydroxymethyl piperidine and a solution of 4-fluorobenzonitrile (1.2 eq) in DMF (75 ml) and stirred 18 h at AT. An aqueous 1 N NaOH solution is added, extraction with DCM, the organic layer is dried over MgSO₄, filtered, evaporated in vacuo and the residue obtained is washed with pentane. 30.6 g of desired product are obtained, which is used as such.

### B/ 4-(Piperidin-3-ylmethoxy)-benzonitrile

10 g of desired product are isolated after following General Procedure C to deprotect 10.8 g of compound obtained in the preceding step.

### C/ 4-(1-Isopropyl-piperidin-3-ylmethoxy)-benzonitrile

A mixture of 6 g of product obtained in the previous step, 1.7 g of acetone and 5 g of Na₂SO₄ in 50 ml of 1,2-dichloroethane is stirred 15 h at AT, then 6.64 g of sodium triacetoxyborohydride are added, stirring is continued 48 h at AT, then MeOH is added and the mixture is evaporated in vacuo. The residue is redissolved in DCM, washed with an aqueous ammonia solution, dried over MgSO₄, filtered and evaporated. 3.37 g of desired product are obtained, which is used as such.

### D/ 4-(1-Isopropyl-piperidin-3-ylmethoxy)-benzoic acid

2.25 g of desired product are provided after following General Procedure B to treat 2.28 g of compound obtained in the preceding step, using methoxyethanol as solvent instead of ethanol.

### Preparation 21

### 4-(1-Isopropyl-pyrrolidin-3-yloxy)-benzoic acid

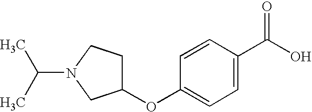

### A/ 4-(1-Isopropyl-pyrrolidin-3-yloxy)-benzonitrile

To a solution of NaH (1.95 eq) in DMF (100 ml) is added 22 g of 1-isopropyl-3-pyrrolidinol, heated at 60° C. for 30 min, then a solution of 4-fluorobenzonitrile (1.9 eq) in DMF (56 ml) is added and the whole is heated at 90° C. 14 h. The reaction medium is concentrated to dryness, redissolved in water, extracted with DCM and the organic layer is washed with water and with a saturated NaCl solution, dried over MgSO₄, filtered and concentrated. 4.2 g of desired product are obtained in the form of a yellow oil, after chromatography on silica eluting with a DCM/MeOH/NH₄OH mixture (95:5:0.5 v/v/v).

### B/ 4-(1-Isopropyl-pyrrolidin-3-yloxy)-benzoic acid

1.6 g of desired product are obtained in powder form, by treating the compound obtained in the preceding step following General Procedure B.

### Preparation 22

### 4-(1-Isopropyl-piperidin-3-yloxy)-benzoic acid

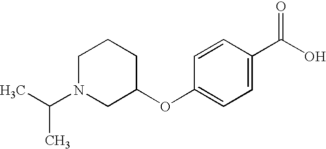

### A/ 1-Isopropyl-(3-piperidinol

To a mixture of 3-hydroxypiperidine (15 g) and Na₂SO₄ (10 g) in chloroform (400 ml) is added 1 eq acetone, the reaction medium is stirred 12 h at AT, then sodium triacetoxyborohydride (1.9 eq) is added and stirred 24 h at AT, after evaporation in vacuo and washing the residue several times with acetone, 25 g of desired product are obtained, which is used as such.

### B/ 4-(1-Isopropyl-piperidin-3-yloxy)-benzonitrile

7.6 g of product obtained in the previous step are solubilized in DMF (20 ml), added to a suspension of NaH (3 eq) in DMF (50 ml), stirred 1 h at AT, then 4-fluorobenzonitrile (0.9 eq) in DMF (5 ml) is added and stirred 48 h at AT. The reaction medium is evaporated to dryness, the residue redissolved in TBME, the organic layer is washed with water, dried over MgSO₄, filtered and concentrated. 5.8 g of desired product are isolated in solid form, after chromatography on silica eluting with a DCM/MeOH/NH₄OH mixture (90:10:0.1 v/v/v).

### C/ 4-(1-Isopropyl-piperidin-3-yloxy)-benzoic acid

5.7 g of desired product are obtained in powder form by treating the compound obtained in the preceding step following General Procedure B.

### Preparation 23

### 3-(1-Isopropyl-piperidin-3-yloxy)-benzoic acid

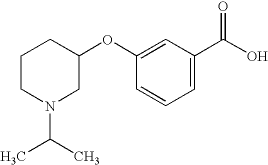

### A/ 3-(1-Isopropyl-piperidin-3-yloxy)-benzonitrile

8.6 g of desired product are isolated in solid form, following the operating mode described in Preparation 22 step B, from 13.1 g of 1-isopropyl-(3-piperidinol obtained such as described under Preparation 22 step A, in the presence of 3-fluorobenzonitrile (1.5 eq).

### B/ 3-(1-Isopropyl-piperidin-3-yloxy)-benzoic acid

The desired product is obtained in powder form (10 g), by following General Procedure B to treat 11 g of compound obtained as described in the preceding step.

### Preparation 24

### 3-(1-Isopropyl-piperidin-4-yloxy)-benzoic acid

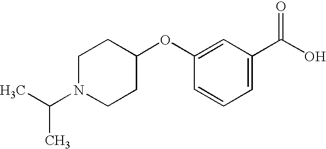

### A/ 3-(1-Isopropyl-piperidin-4-yloxy)-benzonitrile

7.2 g of 1-isopropyl-piperidin-4-ol obtained as described in Preparation 2, step A, are solubilized in DMF (20 ml), this solution is added to a suspension of NaH (3.4 eq) in DMF (50 ml) and stirred 1 h at AT, then 3-fluorobenzonitrile (1.2 eq) in 5 ml DMF is added and heated for 14 h at 60° C. The reaction medium is concentrated to dryness, the residue obtained redissolved in TBME, washed with water, the organic layer is dried over MgSO₄, filtered and concentrated. 4.5 g of desired product are isolated in solid form after chromatography on silica eluting with a DCM/MeOH/NH₄OH mixture (90:10:0.1 v/v/v).

### B/ 3-(1-Isopropyl-piperidin-4-yloxy)-benzoic acid

4.5 g of desired product are obtained in powder form by treating the compound obtained in the preceding step, following General Procedure B.

### Preparation 25

### 4-(2,2,6,6-Tetramethyl-piperidin-4-yloxy)-benzoic acid

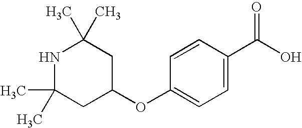

### A/ 4-(2,2,6,6-Tetramethyl-piperidin-4-yloxy)-benzonitrile

To a solution of 4-hydroxy-2,2,6,6-tetramethylpiperidine (10 g) in DMF (100 ml) is added NaH (3 eq) and stirred 1 h at AT, followed by the addition of a 4-fluorobenzonitrile solution (1 eq) in DMF (10 ml) and further stirring for 4 h at AT. The DMF is evaporated in vacuo, the residue is redissolved in water and the formed precipitate is filtered and dried. 19 g of desired product are obtained, which is used as such.

### B/ 4-(2,2,6,6-Tetramethyl-piperidin-4-yloxy)-benzoic acid

14.9 g of desired product are obtained by treating the compound obtained in the preceding step, following General Procedure B.

### Preparation 26

### 4-(1,2,2,6,6-Pentamethyl-piperidin-4-yloxy)-benzoic acid

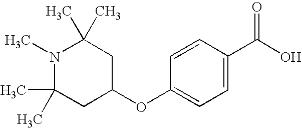

### A/ 1,2,2,6,6-Pentamethyl-piperidinol

To a solution of 2,2,6,6-tetramethyl-4-hydroxypiperidine (6.3 g) in MeOH (16 ml) is added methyl iodide (5 eq) dropwise and stirred at AT 24 h. 64 ml diethyl ether are then added to the reaction medium, the crystals formed are filtered, dissolved in base water, the product extracted with TBME and evaporated. 3.4 g of desired product are obtained.

### B/ 4-(1,2,2,6,6-Pentamethyl-piperidin-4-yloxy)-benzonitrile

To a suspension of NaH (1.95 eq) in DMF (100 ml) is added 8 g of compound obtained as described in the preceding step, heated at 40° C. for 1 h, 4-fluorobenzonitrile (1 eq) in DMF (100 ml) is then added and stirring continued at AT for a further 24 h. The solvent is evaporated to dryness, the residue redissolved in acid water, washed with TBME, basified with an aqueous 1N NaOH solution, extracted with TBME, the organic layer is dried over MgSO4, filtered and evaporated. 11 g of desired product are isolated.

### C/ 4-(1,2,2,6,6-Pentamethyl-piperidin-4-yloxy)-benzoic acid

7 g of desired product are isolated by gollowing General Procedure B to treat the compound obtained in the preceding step.

### Preparation 27

### 4-(8-Methyl-8-aza-bicyclo[3.2.1]oct-3-yloxy)-benzoic acid

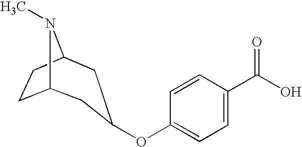

### Method I

### A/ 4-(8-Methyl-8-aza-bicyclo[3.2.1]oct-(3-endo)-yloxy)-benzonitrile

To a suspension of NaH (3 eq) in DMF (290 ml), is added tropine (29 g), heated 1 h at 45° C., then 4-fluorobenzonitrile (1 eq) in DMF (100 ml) is added, stirred 24 h at AT, the solvent evaporated dry, the residue redissolved in water, extracted with DCM. The organic layer is washed with an aqueous 1 N NaOH solution, then an aqueous NaCl solution, dried over MgSO₄, filtered, evaporated. 44 g of desired product are obtained in powder form.

### B/ 4-(8-Methyl-8-aza-bicyclo[3.2.1]oct-(3-endo)-yloxy)-benzoic acid

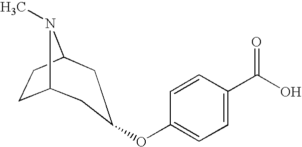

29 g of desired product are isolated in powder form, by following General Procedure B to treat 30 g of the compound obtained in the preceding step.

### Method II

### A/ 4-(8-Methyl-8-aza-bicyclo[3.2.1]oct-(3-exo)-yloxy)-methyl benzoate

To a solution of tropine (10 g) in THF (200 ml) are added 1.4 eq of methyl hydroxybenzoate and 1.4 eq triphenylphosphine then 1.4 eq DIAD keeping the temperature to below 40° C., the reaction medium is stirred 48 h at AT, concentrated, redissolved in diethyl ether, filtered and the filtrate evaporated to dryness. 7 g of desired product are isolated in white solid form, after chromatography on silica eluting with a DCM/MeOH/NH₄OH mixture (95:5:0.5 v/v/v).

### B/ 4-(8-Methyl-8-aza-bicyclo[3.2.1]oct-(3-exo)-yloxy)-benzoic acid

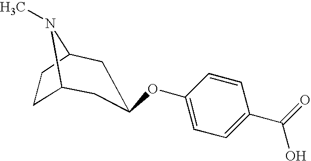

0.5 g of compound obtained in the preceding step are treated with concentrated HCl (1 ml) in water (10 ml) by heating under reflux for 10 h. The reaction medium is cooled, the formed precipitate is filtered, washed with acetone and dried. 300 mg of product are obtained in hydrochloride form.

### Preparation 28

### 3-Fluoro-4-(8-methyl-8-aza-bicyclo[3.2.1]oct-(3-endo)-yloxy)-benzoic acid

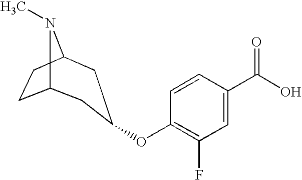

### A/ 3-Fluoro-4-(8-methyl-8-aza-bicyclo[3.2.1]oct-(3-endo)-yloxy)-benzonitrile

A solution of NaH (1.3 eq) in DMF (20 ml), to which tropine (5 g) in DMF (30 ml is added, is heated 30 min at 60° C., then 3,4-difluorobenzonitrile (1 eq) in DMF (10 ml) is added, heated for 4 h at 60° C. and the solvent is evaporated dry. The residue is redissolved in water and extracted with TBME, the organic phase is dried over MgSO₄, filtered and evaporated. 3.2 g of desired product are isolated in white solid form, after chromatography on silica eluting with a DCM/MeOH/NH₄OH mixture (95:5:0.05 v/v/v).

### B/ 3-Fluoro 4-(8-methyl-8-aza-bicyclo[3.2.1]oct-(3-endo)-yloxy)-benzoic acid

Following General Procedure B, the desired product is isolated in the form of a white powder (3.4 g containing minerals) by treating the compound obtained in the preceding step. The product is used as such.

### Preparation 29

### 2-Chloro-4-(8-methyl-8-aza-bicyclo[3.2.1]oct-(3-endo)-yloxy)-benzoic acid

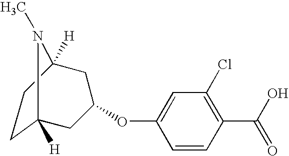

### A/ 2-Chloro-4-(8-methyl-8-aza-bicyclo[3.2.1]oct-(3-endo)-yloxy)-benzonitrile

To a solution of NaH (1.3 eq) in DMF (5 ml), is added 1.82 g tropine (1 eq), stirred 1 h at AT, then a solution of 2 g of 3-fluoro-2-chlorobenzonitrile (1 eq) in DMF (1 ml) is added and stirring continued at AT for 5 h. The reaction medium is diluted with water, extracted with DCM, the organic layer is washed several times with water, dried over MgSO₄, filtered and concentrated to dryness. 272 mg of desired product are isolated after chromatography on silica eluting with a DCM/MeOH/NH₄OH mixture (95:5:0.05 v/v/v).

### B/ 2-Chloro-4-(8-méthyl-8-aza-bicyclo[3.2.1]oct-(3-endo)-yloxy)-benzoic acid

Following general Procedure B, 371 mg of desired product are isolated by treating 705 mg of compound obtained such as described in the preceding step.

### Preparation 30

### 3-Chloro 4-(8-methyl-8-aza-bicyclo[3.2.1]oct-(3-endo)-yloxy)-benzoic acid

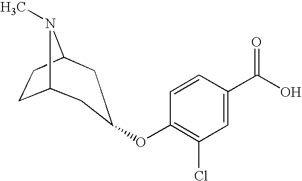

### A/ 3-Chloro(8-methyl-8-aza-bicyclo[3.2.1]oct-(3-endo)-yloxy)-benzonitrile

A suspension of NaH (1.95 eq) in DMF (100 ml), to which tropine (6.3 g) is added, is heated under stirring 1 h at 45° C., then 3-chloro-4-fluoro-benzonitrile (1 eq) in DMF (100 ml) is added and the reaction medium is stirred a further 24 h at AT. The solvent is evaporated to dryness, the residue redissolved in water and extracted with DCM, the organic layer is washed with an aqueous 1 N NaOH solution then an aqueous NaCl solution, dried over MgSO₄, filtered and evaporated. 7.9 g of desired product are obtained in powder form.

### B/ 3-Chloro-4-(8-methyl-8-aza-bicyclo[3.2.1]oct-(3-endo)-yloxy)-benzoic acid

9.5 g of desired product are obtained in hydrochloride form, by treating the compound, obtained during the preceding step, in accordance with General Procedure B. This product containing mineral salts is used as such.

### Preparation 31

### 3-Methoxy-4-(8-methyl-8-aza-bicyclo[3.2.1]oct-(3-endo)-yloxy)-benzoic acid

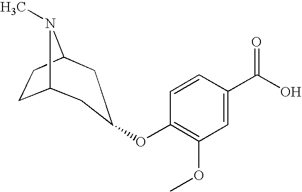

### A/ 3-Methoxy-4-(8-methyl-8-aza-bicyclo[3.2.1]oct-(3-endo)-yloxy)-benzonitrile

A suspension of NaH (1.95 eq) in DMF (100 ml), to which tropine (4.6 g) is added, is heated 1 h at 45° C., then 4-fluoro-3-methoxybenzonitrile (1 eq) in DMF (100 ml) is added and the reaction medium is stirred a further 24 h at AT. The solvent is evaporated to dryness, the residue redissolved in water and extracted with TBME, the organic layer is washed with an aqueous 1 N NaOH solution then an aqueous NaCl solution, dried over MgSO₄, filtered and evaporated. 3.7 g of desired product are obtained in powder form.

### B/ 3-Methoxy-4-(8-methyl-8-aza-bicyclo[3.2.1]oct-(3-endo)-yloxy)-benzoic acid

2.6 g of desired product are obtained by treating the compound, obtained during the preceding step, in accordance with General Procedure B.

### Preparation 32

### 2-Fluoro-4-(8-methyl-8-aza-bicyclo[3.2.1]oct-(3-endo)-yloxy)-benzoic acid

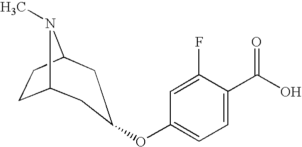

### A/ 2-Fluoro-4-(8-méthyl-8-aza-bicyclo[3.2.1]oct-(3-endo)-yloxy)-benzonitrile

A suspension of NaH (1.95 eq) in DMF (400 ml), to which tropine (10 g) is added, is heated 1 h at 45° C., then 2,4-difluorobenzonitrile (1 eq) in DMF (100 ml) is added and the reaction medium stirred a further 24 h at AT. The solvent is evaporated to dryness, the residue redissolved in acid water and washed with TBME, the aqueous phase is basified, extracted with TBME, dried over MgSO₄, filtered and evaporated. The residue is redissolved in an acetone/aqueous hydrochloric acid mixture, the formed crystals are separated, the filtrate is collected, evaporated in vacuo, redissolved in base water, further extracted with TBME, dried over MgSO₄, filtered and evaporated. 2.1 g of desired product are obtained after successive recrystallizations in diisopropyl ether.

### B/ 2-Fluoro-4-(8-methyl-8-aza-bicyclo[3.2.1]oct-(3-endo)-yloxy)-benzoic acid

1.7 g of compound obtained in the preceding step are heated under reflux in 100 ml of a water/concentrated HCl mixture (1:1 v/v) for 35 h, then evaporated to dryness. 0.9 g of desired product are isolated in TFA salt form, after purification of the reaction medium by semi-preparative HPLC.

### Preparation 33

### 4-(2-Methyl-2-aza-bicyclo[2.2.2]oct-5-cis)-yloxy)-benzoic acid

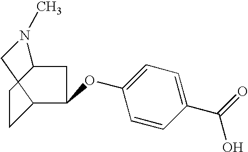

### A/ 2-Carbethoxy-2-azabicyclo[2.2.2]oct-5-ene-2

Following J. Med. Chem. 1973 p. 853, to a solution of 7.7 g BF₃ etherate and 42 g of methylene diurethane in toluene (280 ml) at 80° C., is added dropwise a solution of cyclohexane diene (17.5 g) in toluene (35 ml), and stirred 1 h at 80° C. The reaction medium is then poured onto ice in a mixture with an aqueous NaHCO₃ solution, extracted with toluene and the organic layer is evaporated in vacuo. 38.4 g of desired product are isolated after distilling at 3 mm Hg (Boiling T=75-95° C.).

### B/ 2-Carbethoxy-5,6-epoxy-2-azabicyclo[2.2.2]octane

To the compound obtained in the preceding step in solution in DCM (900 ml), is added meta chloroperbenzoic acid (220 mmol at 70%) and the reaction medium stirred 48 h at AT. The medium is filtered, washed with aqueous NaHCO₃ solution, stirred 48 h in the presence of water/Na₂SO₃ then 48 h in the presence of animal black to remove the peroxides, the organic layer is separated and evaporated. 20.7 g of desired product are isolated after chromatography on silica eluting with a DCM/ethanol mixture (95:5 v/v).

### C/ 2-Methyl-(5-cis)-hydroxy-2-azabicyclo[2.2.2]octane

To the compound obtained in the preceding step, in solution in toluene (100 ml), is added Red-A1 dropwise (130 ml of a 70% solution in toluene), the reaction medium is heated 4 h at 80° C., an ethanol/water mixture is added, followed by filtration and concentration. The residue is redissolved in water, extracted with TBME, and the organic layer is dried over MgSO₄, filtered and concentrated. 2.3 g of desired product are isolated after distilling at 20 mm Hg (Boiling T=95-100° C.; lit. 96-99° C.).

### D/ 4-(2-Methyl-2-aza-bicyclo[2.2.2]oct-(5-cis)-yloxy)-benzonitrile

To a suspension of NaH (1.95 eq) in DMF (100 ml) is added the compound obtained in the preceding step, heated 1 h at 45° C., 4-fluorobenzonitrile (1 eq) in DMF (100 ml) is added and heated 8 h at 45° C. The solvent is evaporated dry, the residue redissolved in water, extracted with TBME then with DCM, dried over MgSO₄, filtered and evaporated. 0.8 g of desired product are isolated after purifying by semi-preparative HPLC.

### E/ 4-(2-Methyl-2-aza-bicyclo[2.2.2]oct-(5-cis)-yloxy)-benzoic acid

The desired product is obtained by following General Procedure B to treat 1.3 g of compound obtained such as described in the preceding step.

### Preparation 34

### 4-(8-Methyl-8-aza-bicyclo[3.2.1]oct-6-yloxy)-benzoic acid

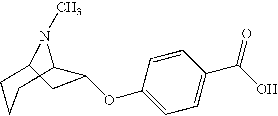

### A/ 6Hydroxymethyl-8-aza-bicyclo[3.2.1]octan-3-one

Following J. Med. Chem., 2000, 43 (17) p 3289, a mixture of 106 g 2,5-dimethoxy-2,5-dihydrofurane cis/trans in an aqueous 3 N HCl solution (1.40 l) is stirred 20 h at AT, then the medium is neutralized with an aqueous 6N NaOH solution, and the whole is added to a mixture of 240 g of 1,3-acetone-dicarboxylic acid, 560 g of anhydrous sodium acetate and 111.6 g methylamine hydrochloride, and stirred 48 h at AT. 1900 g of solid K₂CO₃ are added slowly then a saturated aqueous NaCl solution, followed by extraction in fractions with DCM and evaporation of the organic layer. 28 g of desired product are isolated after chromatography on silica eluting with a DCM/MeOH/NH₄OH mixture (90:10:0.1 v/v/v).

### B/ 8-Methyl-8-aza-bicyclo[3.2.1]octan-6-ol

11.5 g of compound obtained in the preceding step in solution in ethylene glycol (50 ml) are heated under reflux 2 h in the presence of hydrazine hydrate (23 ml), KOH (5 eq) is added and refluxed 2 h. After adding water and extracting with TBME, the organic layer is dried over MgSO₄, filtered and concentrated. 7 g of desired product are obtained.

### C/ 4-(8-Methyl-8-aza-bicyclo[3.2.1]oct-6-yloxy)-benzonitrile

To a solution of NaH (1.95 eq) in DMF (100 ml) is added the compound obtained in the preceding step and heated 1 h at 45° C., then 4-fluorobenzonitrile (1.5 eq) in DMF (100 ml) is added and stirred at AT for 24 h. The solvent is evaporated to dryness, the residue redissolved in water, extracted with TBME, the organic layer is washed with an aqueous 1 N NaOH solution then with an aqueous NaCl solution, dried over MgSO₄, filtered and evaporated. 4.4 g of desired product are obtained.

### D/ 4-(8-Methyl-8-aza-bicyclo[3.2.1]oct-6-yloxy)-benzoic acid

Following General Procedure B, 2.6 g of desired product are obtained by treating the compound obtained in the preceding step, using methoxyethanol as solvent instead of ethanol.

### Preparation 35

### 4-(1-Isobutyl-1,2,3,6-tétrahydro-pyridin-4-yl)-benzoic acid

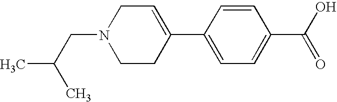

### Method I

### A/ 4-Bromo-N-(2-hydroxy-1,1-dimethylethyl)-benzamide

To a mixture of 2-amino-2-methyl-1-propanol (1.09 eq) and TEA (1.09 eq) in THF (350 ml) is added dropwise at 0° C. a solution of 4-bromobenzoyl chloride (51.5 g) in THF (100 ml) and stirred 18 hours at AT. After concentration in vacuo, the residue is redissolved in DCM, washed with an aqueous 1 N HCl solution then with an aqueous NaHCO₃ solution, the organic phase is dried over MgSO₄, filtered and evaporated. The residue is redissolved in diisopropyl ether, and the precipitate obtained is filtered and dried. 57.4 g of desired product are isolated.

### B/ 2-(4-Bromo-phenyl)-4,4-dimethyl-4,5-dihydro-oxazole

To the compound of the preceding step (57 g), SOCl₂ (3.2 eq) is added dropwise, stirred 4.5 h at AT, then the reaction medium is poured onto anhydrous diethyl ether. The precipitate obtained is filtered, redissolved in an aqueous NaOH solution and extracted with diethyl ether, the organic layer is dried over K₂CO₃, filtered and evaporated. 49.3 g of desired product are obtained.

### C/ 1-Benzyl-4-[4-(4,4-dimethyl-4,5-dihydro-oxazol-2-yl)-phenyl]-piperidin-4-ol

In an inert atmosphe, a solution of the compound obtained in the preceding step (49.3 g) in THF (400 ml), is added to a solution of Mg (1.2 eq) in THF (60 ml), in the presence of a catalytic quantity of iodine. The reaction medium is heated under reflux 3 h, cooled to AT and a solution of benzylpiperidone (1.1 eq) in THF (100 ml) is added carefully whilst keeping the temperature to below 40° C. The reaction medium is heated under reflux for a further 3.5 h followed by the addition of a saturated NH₄Cl solution, extraction with TBME, the organic layer is dried over MgSO₄, filtered and evaporated in vacuo. 46.4 g of desired product are obtained in the form of a yellow solid.

### D/ 4-(1-Benzyl-4-hydroxy-piperidin-4-yl)-ethylbenzoate

15 g of compound obtained in the preceding step are heated under reflux in ethanol (900 ml), in the presence of sulfuric acid (75 ml) for 72 h. After concentration in vacuo, the residue is redissolved in DCM, the organic layer is washed with saturated aqueous NaCl then NaHCO₃ solutions, dried on MgSO₄, filtered and evaporated. 13.3 g of desired product are obtained in oil form.

### E/ 4-(4-Hydroxy-piperidin-4-yl)-ethylbenzoate

Following General Procedure D, 8 g of desired product are obtained in the form of a white powder, from the compound of the preceding step.

### F/ 4-(1,2,3,6-Tetrahydro-pyridin-4-yl)-ethylbenzoate

The compound obtained in the preceding step is heated under reflux for 24 h in ethanol (200 ml) in the presence of H₂SO₄ (50 ml). After concentration in vacuo the residue is redissolved in an aqueous NaCl solution, basified, extracted with ethyl acetate and the organic layer is dried over MgSO₄, filtered and evaporated. The oil obtained is redissolved in an aqueous 1 N HCl solution, washed with TBME, the aqueous phase is basified followed by DCM extraction. This last organic layer is dried over MgSO₄, filtered and evaporated. 4.3 g of desired product are obtained in powder form.

### G/ 4-(1-Isobutyl-1,2,3,6-tetrahydro-pyridin-4-yl)-ethylbenzoate

1 g (4 mmol) of compound obtained in the preceding step in 10 ml DMF is heated at 80° C. for 4 h in the presence of 2.4 eq of isobutyl bromide and 3 eq of K₂CO₃. After concentration in vacuo, the residue is redissolved in water, extracted with DCM, the organic layer is dried over MgSO₄, filtered and evaporated. 0.6 g of desired product are isolated after chromatography on silica eluting with a DCM/MeOH/NH₄OH mixture (95:5:0.05 v/v/v).

### H/ 4-(1-Isobutyl-1,2,3,6-tetrahydro-pyridin-4-yl)-benzoic acid

Following General Procedure A, 0.3 g of desired product are obtained in the form of a white solid from the compound of the preceding step.

### Method II

### A/ 4-(4-(Hydroxy-1-isobutyl-piperidin-4-yl)-ethylbenzoate

1.6 g of compound obtained according to Method I step E are solubilized in DMF (16 ml) and heated at 85° C. for 10.5 h in the presence of 2 eq of isobutyl bromide and 3 eq of K₂CO₃. After concentration in vacuo, the residue is redissolved in water, extracted with TBME and the organic layer is dried over MgSO₄, filtered and evaporated. 1.6 g of desired product are obtained in the form of an orange oil.

### B/ 4-(1-Isobutyl-1,2,3,6-tetrahydropyridin-4-yl)-benzoic acid

The compound obtained in the preceding step is heated 12 h at 100° C. in an acetic acid (8 ml)/concentrated HCl (3.2 ml) mixture, left to cool to AT, the precipitate obtained is filtered and dried. 1.3 g of desired product are isolated in the form of a white solid.

### Method III

### A/ 4-[4-(4,4-Dimethyl-4,5-dihydro-oxazol-2-yl)-phenyl]-piperidin-4-ol

Following General Procédure D, 4.1 g of desired product are obtained from 11.0 g of compound obtained such as described under Method I, step C.

### B/ 4-[4-(4,4-Dimethyl-4,5-dihydro-oxazol-2-yl)-phenyl]-1-isobutyl-piperidin-4-ol

The compound obtained in the preceding step is heated at 50° C. for 7 h in DMF (40 ml) in the presence of 1.2 eq of isobutyl bromide and 2.5 eq of K₂CO₃. After concentration in vacuo, the residue is redissolved in water, the precipitate formed is filtered, dissolved in DCM, and the organic layer is dried over MgSO₄, filtered and evaporated. 2.1 g of desired product are obtained in the form of a white solid.

### C/ N-(2-Hydroxy-1,1-dimethyl-ethyl)-4-(4-hydroxy-1-isobutyl-piperidin-4-yl)-benzamide

The compound obtained in the preceding step is heated under reflux for 24 h in the presence of concentrated HCl (3.2 ml) and water (1.6 ml). After concentration in vacuo, the residue is precipitated in acetone, the precipitate is filtered and dried. 2.4 g of desired product are obtained in the form of a white solid.

### D/ 4-(1-Isobutyl-1,2,3,6-tetrahydropyridin-4-yl)-benzoic acid

The compound obtained in the preceding step is heated under reflux in acetic acid for 16 h, in the presence of concentrated HCl (5 ml). The reaction medium is diluted with acetone, the precipitate obtained is filtered and dried. 1.4 g of desired product are isolated.

### Preparation 36

### 4-(1-Isopropyl-1,2,3,6-tetrahydro-pyridin-4-yl)-benzoic acid

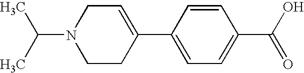

### Method I

### A/ 4-[4-(4,4-Dimethyl-4,5-dihydro-oxazol-2-yl)-phenyl]-1-isopropyl-piperidin-4-ol

A mixture of 4 g of compound obtained such as described under Preparation 35, Method III, step A, acetic acid (9 eq) and acetone (14 eq) in 30 ml of MeOH is stirred 1 h at AT, sodium cyanoborohydride (7 eq) is added and heated at 30° C. for 8 h, concentrated to dryness and the residue redissolved in ethyl acetate/water. The organic layer is separated, washed 3 times with a NaCl saturated solution, dried over MgSO₄, filtered and evaporated. 1.4 g of desired product are isolated in crystal form after chromatography on silica eluting with a DCM/MeOH/NH₄OH mixture (90:10:0.5 v/v/v).

### B/ 4-(1-Isopropyl-1,2,3,6-tetrahydro-pyridin-4-yl)-benzoic acid

In a mixture of concentrated HCl (8 ml)/acetic acid (20 ml), are placed in solution 3.7 g (11.7 mmol) of intermediate obtained such as described under step A, and heated at 100° C. for 24 h. The reaction medium is cooled to AT, diluted with acetone and the precipitate obtained is filtered, rinsed with acetone and dried. 1.9 g of desired product are isolated in white powder form.

### Method II

### A/ 4-[4-(4,4-Dimethyl-4,5-dihydro-oxazol-2-yl)-phenyl]-1-isopropyl-piperidin-4-ol

A mixture of 9.5 g of intermediate obtained such as described under Preparation 35, Method III, step A, of K₂CO₃ (2 eq.) and of 2-bromopropane (1 eq) in 100 ml DMF is heated at 55° C. for 16 h. The solvent is evaporated in vacuo, the residue redissolved in ethyl acetate, the organic layer is washed with a saturated aqueous NaCl solution, dried over MgSO₄, filtered and evaporated. 6.2 g of desired product are isolated in the form of a beige solid after precipitating the residue in pentane.

### B/ N-(2-Hydroxy-1,1-dimethyl-ethyl)-4-(4-hydroxy-1-isopropyl-piperidin-4-yl)-benzamide

2 g of compound obtained in the preceding step are heated under reflux 24 h in a mixture of concentrated HCl (3 ml)/water (1.6 ml). The reaction medium is evaporated and the residue treated with TBME. 2 g of desired product are isolated in powder form.

### C/ 4-(1-Isopropyl-1,2,3,6-tetrahydro-pyridin-4-yl)-benzoic acid

1.8 g of compound obtained in the preceding step are heated 32 h under reflux in a mixture of concentrated HCl (5 ml)/acetic acid (15 ml). The reaction medium is diluted in acetone and the precipitate formed is filtered and dried. 640 mg of desired product are isolated in the form of a white powder.

### Method III

### A/ 4-(4-Hydroxy-1-isopropyl-piperidin-4-yl)-ethylbenzoate

A mixture of 2 g of intermediate obtained such as described under Preparation 35, Method I, step E and of 2.7 eq of 2-bromopropane and 3.5 eq of K₂CO₃, in suspension in 20 ml DMF is heated at 50° C. for 12 h. The reaction medium is concentrated, the residue redissolved in water, extracted with DCM and the organic layer is dried over MgSO₄, filtered and evaporated. The residue is redissolved in pentane, concentrated, redissolved in diethyl ether, washed with water and the organic layer is dried on MgSO₄, filtered and evaporated. 1.3 g of desired product are obtained.

### B/ 4-(1-Isopropyl-1,2,3,6-tetrahydro-pyridin-4-yl)-benzoic acid

The compound obtained in the preceding step is heated under reflux for 7 h in a mixture of acetic acid (10 ml)/concentrated HCl (4 ml). The reaction medium is diluted with acetone, and the precipitate formed is filtered and dried. 1 g of desired product is obtained in the form of a white powder.

### Preparation 37

### 4-[1-(2-Dimethylamino-acetyl)-1,2,3,6-tetrahydro-pyridin-4-yl]-benzoic acid

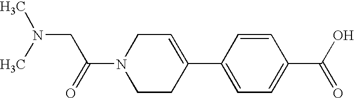

### A/ 4-[1-(2-Dimethylamino-acetyl)-4-hydroxy-piperidin-4-yl]-ethylbenzoate

2 g of intermediate obtained such as described under Preparation 35, Method I, step E is solubilized in DCM (20 ml) and is reacted with N,N′-dimethylglycine (1.5 eq) in the presence of HOBT (1.8 eq), EDCI (1.8 eq) and DIEA (4.1 eq) for 6 h at 60° C. The reaction medium is then evaporated in vacuo, redissolved in water, basified with an aqueous ammonia solution, extracted with TBME and the organic layer is dried over MgSO₄, filtered and concentrated to dryness. 540 mg of desired product are obtained in oil form.

### B/ 4-[1-(2-Dimethylamino-acetyl)-1,2,3,6-tetrahydro-pyridin-4-yl]-benzoic acid

1.14 g of compound obtained such as described under step A is heated 72 h at 100° C. in an acetic acid (23 ml)/concentrated HCl (2.1 ml) mixture. The solvent is then evaporated in vacuo, the residue redissolved in acetone and the precipitate filtered, rinsed with pentane and dried. 420 mg of desired product are obtained in white powder form.

### Preparation 38

### 4-(1-Methyl-1,2,3,6-tetrahydro-pyridin-4-yl)-benzoic acid

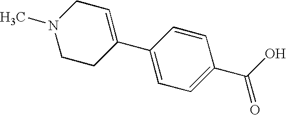

### A/ 4-(4-Hydroxy-1-methyl-piperidin-4-yl)-ethylbenzoate

1.5 g of compound obtained such as described under Preparation 35, Method I, step E is placed in solution in formic acid (5.6 eq) in the presence of formaldehyde (37% solution; 1.5 ml), heated under reflux for 24 h then the reaction medium is concentrated. The residue is redissolved in water, basified, extracted with ethyl acetate and the organic layer is dried over MgSO₄, filtered, concentrated and the oil obtained is precipitated in pentane and the precipitate is filtered and dried. 940 mg of desired product are obtained.

### B/ 4-(1-Methyl-1,2,3,6-tetrahydro-pyridin-4-yl)-benzoic acid

The product isolated in the preceding step is heated under reflux for 4 h in a mixture of acetic acid (15 ml)/concentrated HCl (5 ml), evaporated, redissolved in acetone and the precipitate formed is filtered and dried. 660 mg of desired product are obtained in the form of a white powder.

### Preparation 39

### 4-(1-Ethyl-1,2,3,6-tetrahydro-pyridin-4-yl)-benzoic acid

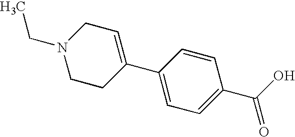

### A/ 4-(1-Ethylhydroxy-piperidin-4-yl)-ethylbenzoate

1.5 g of compound obtained such as described under Preparation 35, Method I, step E is placed in solution in DMF (1.5 ml) in the presence of K₂CO₃ (2.2 eq) and iodoethane (1.2 eq), heated for 3 h at 50° C., the solvent is evaporated in vacuo, the residue redissolved in water, extracted with TBME, and the organic layer is dried over MgSO₄, filtered and concentrated. 1.1 g of desired product are obtained in the form of a white solid.

### B/ 4-(1-Ethyl-1,2,3,6-tetrahydro-pyridin-4-yl)-benzoic acid

The compound obtained in the preceding step is heated 7 h under reflux in a mixture of acetic acid (15 ml)/concentrated HCl (5 ml), the reaction medium is diluted with acetone, and the precipitate obtained is filtered and dried. 1 g of desired product is isolated in the form of a white solid.

### Preparation 40

### 4-(1-Propyl-1,2,3,6-tetrahydro-pyridin-4-yl)-benzoic acid

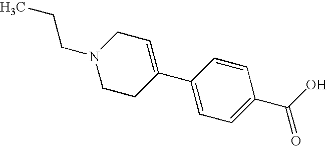

### A/ 4-(4-Hydroxy-1-propyl-piperidin-4-yl)-ethyl benzoate

1 g of compound obtained such as described under Preparation 35, Method I, step E is heated at 65° C. for 4.5 h in the presence of 1.5 eq of 1-bromo-propane and 2.5 eq of K₂CO₃. The reaction medium is concentrated, the residue redissolved in water, extracted with diethyl ether and the organic layer is dried over MgSO₄, filtered and evaporated. 0.97 g of desired product are obtained in the form of a yellow solid.

### B/ 4-(1-Propyl-1,2,3,6-tetrahydro-pyridin-4-yl)-benzoic acid

The compound obtained in the preceding step is heated 12 h under reflux in an acetic acid (5 ml)/concentrated HCl (2.5 ml) mixture, the reaction medium is diluted with acetone, the precipitate obtained is filtered and dried. 0.61 g of desired product is obtained.

### Preparation 41

### 4-(1-Butyl-1,2,3,6-tetrahydro-pyridin-4-yl)-benzoic acid

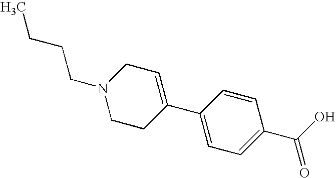

### A/ 4-(1-Butyl-1,2,3,6-tetrahydro-pyridin-4-yl)-ethyl benzoate

1 g of compound obtained such as described under Preparation 35, Method I, step F is heated at 60° C. for 4 h in DMF (10 ml), in the presence of 1.2 eq of 1-bromobutane and 1.5 eq of K₂CO₃. After concentrating the reaction medium, the residue is redissolved in water, extracted with ethyl acetate, and the organic layer is dried over MgSO₄, filtered and evaporated. 0.6 g of desired product are isolated after chromatography on silica eluting with a DCM/MeOH/NH₄OH mixture (95:5:0.05 v/v/v).

### B/ 4-(1-Butyl-1,2,3,6-tetrahydro-pyridin-4-yl)-benzoic acid

Following General Procedure A, 0.4 g of desired product are isolated in the form of a white powder, from the compound obtained in the preceding step.

### Preparation 42

### 4-[1-(3-Methyl-butyl)-1,2,3,6-tetrahydro-pyridin-4-yl]-benzoic acid

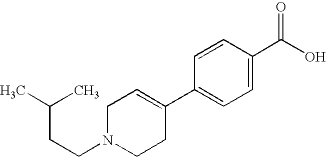

### A/ 4-[1-(3-Methyl-butyl)-1,2,3,6-tetrahydro-pyridin-4-yl]-ethylbenzoate

1 g of compound obtained such as described under Preparation 35, Method I, step F is solubilized in 10 ml DMF and heated at 80° C. for 4 h in the presence of 1.2 eq of 1-bromo-3-methyl-butane and 1.5 eq of K₂CO₃. The reaction medium is concentrated, the residue redissolved in water, extracted with ethyl acetate, and the organic layer is dried over MgSO₄, filtered and evaporated. 0.7 g of desired product are isolated after chromatography on silica eluting with a DCM/MeOH mixture (95:5 v/v).

### B/ 4-[1-(3-Methyl-butyl)-1,2,3,6-tetrahydro-pyridin-4-yl]-benzoic acid

Following General Procedure A, 0.45 g of product are isolated after treating the compound obtained in the preceding step.

### Preparation 43

### 4-(1-Isopropyl-piperidin-4-yl)-benzoic acid

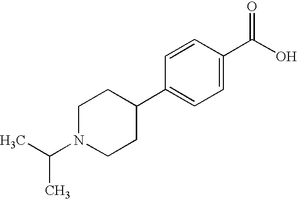

### A/ 4-(1-Benzyl-1,2,3,6-tetrahydro-pyridin-4-yl)-ethyl benzoate

4.5 g of compound obtained such as described under Preparation 35, Method I, step D are heated under reflux for 15 h in 50 ml of anhydrous toluene and in the presence of P₂O₅ (1.6 eq), then the reaction medium is concentrated, the residue redissolved in water, extracted with DCM and the organic layer is washed with water then with an aqueous 1 N NaOH solution, dried over MgSO₄, filtered and evaporated to dryness. 3.3 g of desired product are obtained in powder form.

### B/ 4-Piperidin-4-yl-ethyl benzoate

2.5 g of desired product are obtained in powder form after following General Procedure D to treat the compound obtained in the preceding step.

### C/ 4-(1-Isopropyl-Piperidin-4-yl)-ethyl benzoate

The compound obtained in the preceding step is reacted with acetone (12 eq) and sodium cyanoborohydride (4 eq) in MeOH (21 ml) in the presence of acetic acid (4.7 ml) at 35° C. for 3 h and then 12 h at AT. The medium is concentrated, the residue redissolved in water, basified with an aqueous ammonia solution, extracted with TBME, and the organic layer is dried over MgSO₄, filtered and evaporated. 2.2 g of desired product are obtained in the form of a yellow oil.

### D/ 4-(1-Isopropyl-piperidin-4-yl)-benzoic acid

1.5 g of desired product are obtained in the form of a white solid by following General Procedure A to treat the compound obtained in the preceding step.

### Preparation 44

### 4-(1-Isopropyl-piperidin-4-ylidenemethyl)-benzoic acid

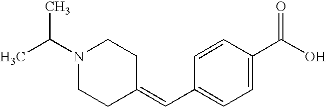

### A/ 4-Diethoxy-phosphorylmethyl)-methyl benzoate

Following Freydante, Tetrahedron, 2002, 58, pp 1425-1432, triethylphosphite (2 eq) and 4-methyl bromomethylbenzoate (12.5 g) are mixed in an inert atmosphere and heated at 160° C. for 4 h. The reaction medium is diluted with DCM, washed with water and the organic layer is dried over MgSO₄, filtered and evaporated in vacuo. 11.2 g of desired product are obtained in the form of a colourless, viscous oil.

### B/ 4-(1-Isopropyl-piperidin-4-ylidenemethyl)-benzoic acid

5 g (16 mmol) of product obtained in the preceding step are solubilized in 25 ml of THF and this solution is added to a suspension of NaH (4.5 eq) in THF (35 ml), cooled to 0° C., followed by the dropwise addition of N-isopropylpiperidinone (1 eq) in THF (25 ml) and stirring for 4 h at AT. The reaction medium is concentrated, the residue redissolved in a DCM/water mixture, acidified to pH5 with an aqueous 4 N HCl solution, brought back to pH 7 with an aqueous NaHCO₃ solution, the aqueous phase is concentrated to dryness and the residue washed with methoxyethanol. 0.5 g of desired product are isolated after crystallization in diethyl ether and washing of the crystals with MeOH.

### Preparation 45

### 4-(1-Isopropyl-piperidin-4-ylmethyl)-benzoic acid

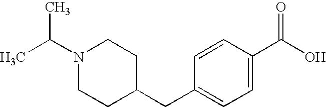

600 mg of compound, obtained such as described under Preparation 44, in solution in MeOH (20 ml) are stirred for 15 h in a hydrogen atmosphere, at AP and in the presence of 10% palladium on charcoal. 0.47 g of desired product are obtained after filtering the catalyst and concentrating the filtrate to dryness.

### Preparation 46

### 1-(3-Piperidin-1-yl-propyl)-1H-indole-5-carboxylic acid

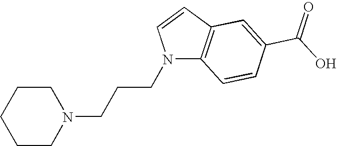

### A/ 1-(3-Chloro-propyl)-1H-indole-5-methyl carboxylate

To a solution of 5-indole methyl carboxylate (1 g) in DMSO (20 ml) are added KOH (1.3 eq) and 1-bromo-3-chloropropane (3 eq) and stirred 50 h at AT. The reaction medium is poured into water, extracted with ethyl acetate, the organic layer is washed with a saturated aqueous NaCl solution, dried over Na₂SO₄, filtered and evaporated. 1.2 g of desired product are isolated in the form of a colourless oil, after purification by chromatography on silica eluting with a 80:20 v/v cyclohexane/ethyl acetate mixture

### B/ 1-(3-Piperidin-1-yl-propyl)-1H-indole-5-methyl carboxylate

The compound obtained in the preceding step is heated in the presence of piperidine (1.5 eq) and DIEA (1.5 eq) in DMF (10 ml) at 90° C. for 14 h, the DMF is evaporated, the residue redissolved in DCM, washed with water, the organic layer is dried over MgSO₄, filtered and evaporated. 960 mg of desired product are obtained in oil form.

### C/ 1-(3-Piperidin-1-yl-propyl)-1H-indole-5-carboxylic acid

General Procedure A is followed to treat the compound obtained in the preceding step. 570 mg of desired product are obtained in the form of a white powder.

### Preparation 47

### 1-[3-(4-Hydroxy-piperidin-1-yl)-propyl]-1H-indole-5-carboxylic acid

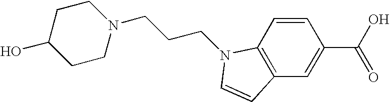

### A/ 1-[3-(4-Hydroxy-piperidin-1-yl)-propyl]-1H-indole-5-methyl carboxylate

1.4 g of compound obtained such as described under Preparation 46, step A are reacted with 4-hydroxypiperidine (1.5 eq), under the conditions described in Preparation 46, step B. 1.44 g of desired product are isolated in the form of a yellow oil following the same treatment as described under Preparation 46, step B.

### B/ 1-[3-(4-Hydroxy-piperidin-1-yl)-propyl]-1H-indole-5-carboxylic acid

General Procedure A is followed to treat the compound obtained in the preceding step. 1.06 g of desired product are obtained in the form of a pink powder.

### Preparation 48

### 1-(2-Piperidin-1-yl-ethyl)-1H-indole-5-carboxylic acid

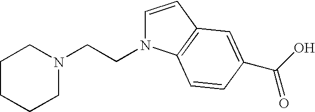

### A/ 1-(2-Piperidin-1-yl-ethyl)-1H-indole-5-methyl carboxylate

A solution of 5-indole methyl carboxylate (4.2 g) in 22 ml DMF is poured onto a suspension of NaH (1.23 eq) in DMF (36 ml), stirred 1 h at AT then 2-chloroethyl piperidine (1.3 eq) in solution in DMF is added, the reaction medium is heated at 55° C. for 2 h and evaporated in vacuo. The residue is redissolved in water, extracted with DCM, the organic layer is washed with an aqueous Na₂CO₃ solution, dried over MgSO₄ and concentrated. To this residue is added a solution of HCl in isopropanol, and the precipitate formed is filtered and dried. 4 g of desired product (white powder) are isolated in hydrochloride form.

### B/ 1-(2-Piperidin-1-yl-ethyl)-1H-indole-5-carboxylic acid

General Procedure A is followed to treat the compound obtained in the preceding step. 2.2 g of desired product are obtained in the form of a yellow powder.

### Preparation 49

### 3-Methyl-1-(2-piperidin-1-yl-ethyl)-1H-indole-5-carboxylic acid

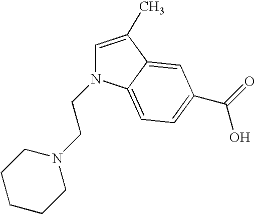

### A/ 4-Amino-3-bromo-ethyl benzoate

To a solution of ethylaminobenzoate (200 mmol) in acetic acid (500 ml) is added dropwise over 3 h a solution of bromine (1 eq) in acetic acid (20 ml), the formed crystals are collected and washed with TBME. 22.6 g of desired product are obtained after chromatography on silica eluting with a DCM/pentane mixture (50:50 v/v).

### B/ 4-Allylamino-3-bromo-ethyl benzoate

The compound obtained in the preceding step is heated under reflux in a mixture of ethanol (400 ml)/water (150 ml), in the presence of NaHCO₃ (2.14 eq) and allyl bromide (2.04 eq) for 5 h, after concentration the residue is redissolved in water, extracted with TBME and the organic layer is dried over MgSO₄, filtered and concentrated to dryness. 6.6 g of desired product are isolated after chromatography on silica eluting with a DCM/pentane mixture (50:50 v/v).

### C/ 3-Methyl-1H-indole-5-ethyl carboxylate

The compound obtained in the preceding step is heated in ACN (120 ml) at 110° C. for 72 h, in the presence of palladium acetate (0.3 eq), ortho-tritolylphosphine (0.3 eq) and TEA (1.5 eq), the reaction medium is concentrated, redissolved in water, extracted with TBME and the organic layer is dried over MgSO₄, filtered and concentrated. 2.5 g of desired product are isolated after purification by chromatography on silica eluting with a DCM/pentane mixture (50:50 v/v).

### D/ 3-Methyl-1-(2-piperidin-1-yl-ethyl)-1H-indole-5-ethyl carboxylate

To a suspension of NaH (1.23 eq) in 20 ml of DMF is added the compound obtained in the preceding step (2.5 g) in solution in DMF (12 ml), and stirred 1 h at AT, then a solution of 2-chloroethyl piperidine (1.3 eq) in 2.5 ml of DMF is added dropwise, heated 2 h at 55° C., the reaction medium is concentrated, redissolved in water, extracted with DCM and the organic layer is dried over Na₂SO₄, filtered and evaporated to dryness. 2.7 g of desired product are isolated in powder form after chromatography on silica eluting with a DCM/MeOH/NH₄OH mixture (98:2:0.2 v/v/v).

### E/ 3-Methyl-1-(2-piperidin-1-yl-ethyl)-1H-indole-5-carboxylic acid

General Procedure A is followed to treat the compound obtained in the preceding step and to isolate 1 g of desired product.

### Preparation 50

### 3-Acetyl-1-(2-piperidin-1-yl-ethyl)-1H-indole-5-carboxylic acid

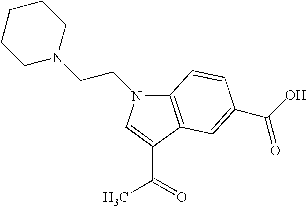

### A/ 3-Acetyl-1H-indole-5-methyl carboxylate

Following the method of Okauchi, Org. Lett., 2000, 2 (10), pp 1485-1488, 5-indole methyl carboxylate (11.4 mmol) in DCM (49 ml) is cooled to 0° C., diethylaluminuim chloride (1.52 eq in 1 M hexane solution) is added and stirred 30 min at 0° C., and then acetyl chloride (3 eq) in solution in DCM (66 ml) is added and stirred 3 h at 0° C. and 48 h at AT. An aqueous buffer solution is poured dropwise onto the reaction medium, the precipitate obtained is filtered and dried with pentane. 2.25 g of desired product are obtained in the form of a pink powder.

### B/ 3-Acetyl-1-(2-piperidin-1-yl-ethyl)-1H-indole-5-methyl carboxylate

450 mg of compound of the preceding step are reacted at 55° C. for 2 h with N-(2-chloroethylpiperidine) (1.3 eq), in the presence of NaH (1.23 eq) in DMF (5.5 ml), then the reaction medium is evaporated. The residue is redissolved in water, extracted with DCM the organic layer is washed with aqueous Na₂CO₃ solution, dried over MgSO₄, filtered and evaporated to dryness. 296 mg of desired product are isolated in powder form after chromatography on silica eluting with a DCM/MeOH/NH₄OH mixture (95:5:0.5 v/v/v).

### C/ 3-Acetyl-1-(2-piperidin-1-yl-ethyl)-1H-indole-5-carboxylic acid

280 mg of desired product are isolated in yellow powder form after following General Procedure A to treat the compound obtained obtained in the preceding step.

### Preparation 51

### 3-(4-Hydroxy-piperidin-1-ylmethyl)-1-isopropyl-1H-indolecarboxylic acid

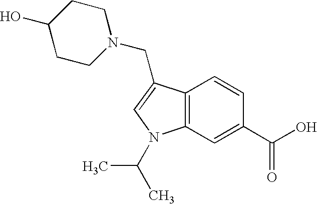

### A/ 1-Isopropyl-1H-indole-5-methyl carboxylate

To a suspension of NaH (1.2 eq) in DMF (32 ml) is added 5 indole-methyl carboxylate (27 mmol) and stirred 30 min at AT, then a solution of isopropyl iodide (1 eq) is added and heated 8 h at 40° C. The reaction medium is evaporated to dryness, the residue redissolved in TBME, washed with water, the organic layer is dried over MgSO₄, filtered and concentrated. 3.7 g of desired product are isolated after chromatography on silica eluting with DCM.

### B/ 3-(4-Hydroxy-piperidin-1-ylmethyl)-1-isopropyl-1H-indole-5-methyl carboxylate

To a solution of 4-hydroxypiperidine (1.7 g) in acetic acid (10 ml) are added dropwise at 5° C. 1.4 ml of 35% formaldehyde in water, then 3.7 g (1 eq) of compound obtained in the preceding step, followed by stirring at AT for 1 h. The reaction medium is poured onto a water/ice mixture, washed with TBME, the aqueous layer is basified, extracted with TBME, and the organic layer is dried over MgSO₄, filtered and evaporated. 5.4 g of desired product are obtained.

### C/ 3-(4-Hydroxy-piperidin-1-ylmethyl)-1-isopropyl-1H-indolcarboxylic acid

3.8 g of desired product are obtained by following General Procedure A to treat the compound obtained in the preceding step.

### Preparation 52

### -[2-(4-Hydroxy-piperidin-1-yl)-ethyl]-benzofuran-6-carboxylic acid

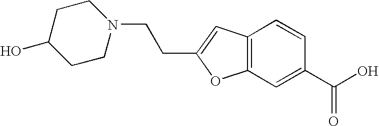

### A/ 3-Hydroxy 4 iodo-methyl benzoate

To a solution of 11.5 g of 4-amino-3-methyl hydroxybenzoate in water (23 ml), is added a solution of concentrated H₂SO₄ (14 ml) in water (46 ml), cooled to between 0 and 5° C., then a solution of NaNO₂ (1.1 eq) in water (14 ml) is added and stirred 1 h keeping the temperature to between 0 and 5° C. Next a solution of KI (1.5 eq) in water (92 ml) is added and stirring continued for 15 h at AT. The reaction medium is extracted with DCM, the organic layer is washed with an aqeuous 10% Na₂S₂O₃ solution and with water, dried over MgSO₄, filtered and evaporated. 7.3 g of desired product are obtained after chromatography on silica eluting with a cyclohexane/ethyl acetate mixture (65:35 v/v).

### B/ But-3-ynyl methanesulfonate

To a mixture of 3-butyn-1-ol (22.4 g) and TEA (1.1 eq) in DCM (250 ml) is added dropwise methane sulfonyl chloride (1.1 eq), stirred 17 h at AT, and evaporated in vacuo. 23.4 g of desired product are obtained after purification by chromatography on silica eluting with a DCM/MeOH mixture (90:10 v/v).

### C/ 1-But-3-ynyl-Piperidin-4-ol

The product obtained in the preceding step is heated under reflux, in solution in DCM (350 ml), in the presence of 4-hydroxypiperidine (2.8 eq) for 48 h. 14.3 g of desired product are obtained after chromatography on silica eluting with a DCM/MeOH mixture (90:10 v/v).

### D/ 3-[2-(4Hydroxy-piperidin-1-yl)-ethyl]-benzofuran-6-methyl carboxylate

A mixture in DMF (40 ml) of 2.67 g of compound obtained in step A, of 1,1,3,3-tetramethylguanidine (1 eq), of compound obtained in step C (2 eq), of bis(triphenylphosphine)palladium chloride (II) (0.1 eq) and of CuI (0.1 eq) is stirred at AT for 96 h. The reaction medium is poured onto a water/ice mixture, extracted with ethyl acetate, the organic layer is washed with water, dried over MgSO₄, filtered and concentrated. 1.8 g of desired product are isolated after chromatography on silica eluting with a DCM/MeOH mixture (94:6 v/v).

### E/ -[2-(4-Hydroxy-piperidin-1-yl)-ethyl]-benzofuran-6-carboxylic acid

1.4 g of desired product are obtained by following General Procedure A to treat the compound obtained in the preceding step.

### Preparation 53

### 1-Isopropyl-2-(2-piperidin-1-yl-ethyl)-1H-benzoimidazole-5-carboxylic acid

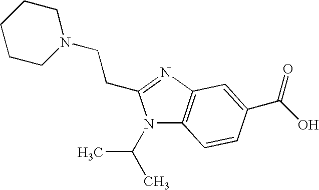

### A/ 4-Isopropylamino-3-nitro-benzoic acid

4-fluoro-3-nitrobenzoic acid (6 g) in DMF (32 ml) are placed in an autoclave, isopropylamine (6 eq) and DIEA (7 eq) are added, the reaction medium is heated at 55° C. for 5 h, the solvent is evaporated in vacuo, the residue is redissolved in an aqueous 1 N HCl solution, the precipitate is filtered, washed with water then oven dried. 7.1 g of desired product are obtained in powder form.

### B/ 3-Amino-4-isopropylamino-benzoic acid

The compound obtained in the preceding step is hydrogenated following General Procedure E. 5.4 g of desired product are obtained.

### C/ 4-Isopropylamino-3-(3-piperidin-1-yl-propionylamino)-benzoic acid

1-piperidinopropanoic acid (2 eq) in DCM (15 ml) is activated in the presence of DCC (1.1 eq), HOBT (1.1 eq) and DIEA (3 eq) 1 h at AT, 0.9 g of product obtained in the preceding step is added and stirred 12 h at AT, the insolubles are filtered, the product extracted with an aqueous 1 N HCl solution, washed with DCM, the aqueous layer is evaporated dry. The desired product is obtained and used as such in the following step.

### D/ 1-Isopropyl-2-(2-piperidin-1-yl-ethyl)-1H-benzoimidazole-5-ethyl carboxylate

The compound obtained in the preceding step is heated at 60° C. for 24 h in the presence of HCl (80 ml of a 2.4 M solution in ether) and ethanol (40 ml). The reaction medium is concentrated, redissolved in water, basified with aqueous NaOH then extracted with DCM. The organic layer is dried over MgSO₄, filtered and evaporated to dryness. 360 mg of desired product are isolated after chromatography on silica eluting with a DCM/EtOH/NH₄OH mixture (90:10:0.5).

### E/ 1-Isopropyl-2-(2-piperidin-1-yl-ethyl)-1H-benzoimidazole-5-carboxylic acid

The compound obtained in the preceding step is heated under reflux for 1 h in a mixture of water (25 ml)/concentrated HCl (50 ml) and then evaporated to dryness. 390 mg of desired product are obtained.

### Preparation 54

### 4-[ethyl-(3-piperidin-1-yl-propionyl)-amino]-benzoic acid

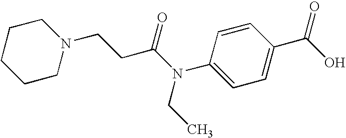

### Method I

### A/ 3-piperidin-1-yl-propionyl chloride

4.1 g of 1-piperidine propionic acid is heated under reflux for 2 h in the presence of SOCl₂ (26 ml), the SOCl₂ is evaporated in vacuo, toluene is added and again evaporated in vacuo. The desired product is obtained in powder form. This product is used without any other purification for the following step.

### B/ 4-Acetylamino-ethyl benzoate

Following Monge, J. Med. Chem., 1995, 38, 10 pp 1786-1792, 4-aminoethylbenzoate (10 g) is acetylated in the presence of acetic anhydride (145 ml/mmol) and acetic acid (50:50 v/v) by heating under reflux for 30 min. The reaction medium is poured on ice and 12.6 g of desired product are isolated in the form of a white powder, after filtering and washing in pentane the precipitate obtained.

### C/ 4-Ethylamino-ethyl benzoate

Following Wakamatsu, Heterocycles, 1980, 14 (10), pp 1437-1440, the acetyl function of the compound obtained in the preceding step (4.1 g) is selectively reduced in the presence of tetra-N-butylammonium borohydride (3 eq), by heating under reflux in DCM for 14 h. The reaction medium is concentrated, redissolved in DCM, the organic layer is washed with an aqueous 3N HCl solution, dried over MgSO₄ and concentrated in vacuo. 2 g of desired product are isolated in the form of a white powder after chromatography on silica eluting with DCM.

### D/ 4-[Ethyl-(3-piperidin-1-yl-propionyl)-amino]-ethyl benzoate

1 g of compound obtained in the preceding step is solubilized in DCM in the presence of TEA (1 eq) and it is added to the acid chloride (1 eq) obtained in step A, in solution in DCM/toluene (50:50 v/v). The reaction medium is stirred 48 h at AT, evaporated in vacuo, redissolved in ethyl acetate, washed with an aqueous Na₂CO₃ solution, dried over MgSO₄, filtered and evaporated in vacuo. 520 mg of desired product are isolated in powder form after chromatography on silica eluting with a DCM/MeOH/NH₄OH mixture (95:5:0.5 v/v/v).

### E/ 4-[ethyl-(3-piperidin-1-yl-propionyl)-amino]-benzoic acid

The desired product is obtained by following the operating mode described under General Procedure A to treat the compound obtained in the preceding step.

### Method II

### A/ 4-[(3-Chloro-propionyl)-ethyl-amino]-ethyl benzoate

To a solution of 5 g compound obtained such as described under Preparation 54, Method I, step C in glacial acetic acid (40 ml), is added 3-chloropropionyl chloride (4 eq), heated 24 h at 35° C., concentrated in vacuo, redissolved in an aqueous solution of sodium acetate, extracted with diethyl ether, and the organic layer is dried over MgSO₄, filtered and evaporated. 8 g of desired product are isolated in the form of a yellow oil after chromatography on silica eluting with the a DCM/acetone mixture (99:1 v/v).

### B/ 4-[Ethyl-(3-piperidin-1-yl-propionyl)-amino]-ethyl benzoate

2 g of compound obtained in the preceding step are heated under reflux in THF (16 ml) for 24 h, in the presence of DIEA (2 eq) and piperidine (2 eq), evaporated to dryness, redissolved in water, extracted with diethyl ether, dried over MgSO₄, filtered and evaporated. 2 g of desired product are obtained in oil form.

### C/ 4-[ethyl-(3-piperidin-1-yl-propionyl)-amino]-benzoic acid

1.5 g of desired product are isolated in the form of a white powder by following General Procedure A to treat the compound obtained in the preceding step.

### Preparation 55

### 4-(3-piperidin-1-yl-propionylamino)-benzoic acid

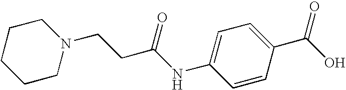

### Method I

### A/ 4-(3-Chloro-propionylamino)-ethyl benzoate

To a solution of 6 g of 4-amino-ethyl benzoate in glacial acetic acid (60 ml) is added 3-chloropropionyl chloride (2.2 eq), heated 8 h at 35° C. then 48 h at AT. The solvent is evaporated in vacuo, the residue redissolved in an aqueous sodium acetate solution, the aqueous layer is extracted with diethyl ether, the organic layer dried over MgSO₄, filtered and evaporated in vacuo. 4.6 g of desired product are isolated in the form of a white powder, after chromatography on silica eluting with a DCM/acetone mixture (99:1 v/v).

### B/ 4-(3-Piperidin-1-yl-propionylamino)-ethyl benzoate

2 g of compound obtained in the preceding step are heated under reflux in THF (16 ml) for 24 h, in the presence of DIEA (2 eq) and piperidine (2 eq). The reaction medium is evaporated to dryness, redissolved in water, the aqueous layer is extracted with diethyl ether, the organic layer is dried over MgSO₄, filtered and evaporated. 2.2 g of desired product are obtained in the form of an oil.

### C/ 4-(3-piperidin-1-yl-propionylamino)-benzoic acid

1.57 g of desired product are isolated in the form of a beige powder by following General Procedure A to treat the compound obtained in the preceding step.

### Method II

### A/ 4-(3-Piperidin-1-yl-propionylamino)-ethyl benzoate

1 g of 4-amino-ethyl benzoate in solution in 20 ml of DCM in the presence of TEA (1.1 ml, 1 eq) is added to the compound obtained such as described under Preparation 54, Method I, step A (1 eq) in solution in 40 ml DCM, and stirred 48 h at AT. After evaporation in vacuo, the residue is redissolved in ethyl acetate, washed with a saturated aqueous Na₂CO₃ solution, and the organic layer is dried over MgSO₄, filtered and evaporated. 340 mg of desired product are isolated in powder form, after chromatography on silica eluting with a DCM/MeOH INH₄OH mixture (95:5:0.5 v/v/v).

### B/ 4-(3-piperidin-1-yl-propionylamino)-benzoic acid

344 mg of desired product are isolated in hydrochloride form by following General Procedure A to treat the compound obtained during the preceding step.

### Preparation 56

### 4-[acetyl-(2-piperidin-1-yl-ethyl)-amino]-benzoic acid

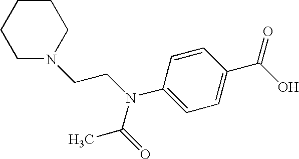

### A/ 4-(2-Piperidin-1-yl-ethylamino)-benzonitrile

4-fluorobenzonitrile (9.5 g), 1-(2-aminoethyl)piperidine (9.5 g, 1 eq) and K₂CO₃ (21 g, 2 eq) are placed in suspension in 15 ml DMF, stirred 24 h at 100° C. then evaporated to dryness. The residue is redissolved in water/DCM, the aqueous layer extracted with DCM the organic layer dried over Na₂SO₄ and evaporated in vacuo. 5.1 g of desired product are isolated after chromatography on silica eluting with an ethyl acetate/cyclohexane mixture (50:50 v/v).

### B/ 4-(2-Piperidin-1-yl-ethylamino)-benzoic acid

The product obtained in the preceding step is solubilized in 220 ml of a mixture of water/EtOH 80:20 (v/v), 8.8 g of NaOH is added, heated under reflux for 96 h, then the solvent is evaporated in vacuo. The residue is redissolved in water, acidified to pH 3 with SO₂ and the water concentrated until a precipitate is formed. 7.2 g of desired product are isolated containing sodium salts, after filtering and washing this precipitate with a mixture of water/EtOH then with MeOH. This product is used as such in the following step.

### C/ 4-[Acetyl-(2-Piperidin-1-yl-ethyl)amino]-benzoic acid

1 g of product from the previous step is placed in solution in pyridine (18 ml) in the presence of acetic anhydride (5 ml) and stirred 4 h at AT. After evaporation to dryness, the residue is redissolved in water, acidified to pH 1 with aqueous HCl, the aqueous layer washed with DCM, evaporated to dryness, and the salts present partly removed by acetone washings of the residue obtained. 700 g of desired product are obtained in powder form.

### Preparation 57

### 4-[acetyl-(3-piperidin-1-yl-propyl)-amino]-benzoic acid

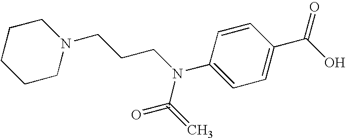

### A/ 4-(3-Piperidin-1-yl-propylamino)-ethyl benzoate

4 g of 3-piperidino-propylamine in DMSO (80 ml) in the presence of TEA (17.6 ml) and 4-ethyl fluorobenzoate (4 eq) are heated at 145° C. for 28 h, the reaction medium is poured into a water/ice mixture, the precipitate formed is filtered and then dissolved in diethyl ether, dried over MgSO₄, filtered and evaporated dry. 4.8 g of desired product are isolated in hydrochloride form after adding a mixture of diethyl ether/HCl, filtering and drying the precipitate formed.

### B/ 4-[Acetyl-(3-Piperidin-1-yl-propyl)-amino]-ethyl benzoate

The compound obtained in the preceding step (8.3 mmol) is heated at 100° C. in a mixture of acetic acid (1.3 ml)/acetic anhydride (1.3 ml) for 3.5 h, then evaporated to dryness. 3 g of desired product are isolated after chromatography on silica eluting with a DCM/MeOH/NH₄OH mixture (90:10:0.05 v/v/v).

### C/ 4-[acetyl-(3-piperidin-1-yl-propyl)-amino]-benzoic acid

Following General Procedure A, 1.95 g of desired product are isolated by treating the compound obtained in the preceding step.

### Preparation 58

### 4-[Acetyl-(3-diethylamino-propyl)-amino]-benzoic acid

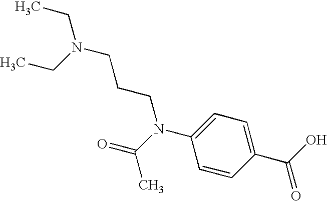

### A/ 4-(3-Diethylamino-propylamino)-benzonitrile

A mixture of 10 g of 4-fluorobenzonitrile, N,N-diethyl-1,3-propanediamine (4 eq) and of K₂CO₃ (2.5 eq) in 100 ml ACN is heated under reflux for 24 h, the insolubles are filtered, the filtrate evaporated. 14 g of desired product are obtained after chromatography on silica, eluting with a DCM/MeOH/NH₄OH mixture (80:20:0.1 v/v/v).

### B/ 4-(3-diethylamino-propylamino)-benzoic acid

Following General Procedure B, 1.6 g of desired product are isolated by treating 7 g of compound obtained in the preceding step.

### C/ 4-(3-Diethylamino-propylamino)-methyl benzoate

A solution of 3 g of product obtained as described in the preceding step is cooled in ice in 90 ml MeOH, then thionyl chloride (3 eq) is added slowly and heated 5 h at 70° C., followed by filtering of the insolubles and evaporation. The residue is redissolved in diethyl ether, filtered, washed with diethyl ether. 2 g of desired product are obtained in powder form.

### D/ 4-[Acetyl-(3-diethylamino-propyl)-amino]-methyl benzoate

A mixture of 1.2 g of compound obtained in the preceding step, of TEA (1 eq) and acetyl chloride (1 eq) in 12 ml DCM is stirred 15 h at AT. After evaporation, the residue is redissolved in an aqueous 2N HCl solution, the aqueous phase is washed with DCM, basified with an aqueous 10% Na₂CO₃ solution, extracted with DCM and the organic layer is dried over MgSO₄, filtered and evaporated. 1 g of desired product is obtained.

### E/ 4-[acetyl-(3-diethylamino-propyl)-amino]-benzoic acid

306 mg of desired product are isolated by following General Procedure A to treat the compound obtained in the preceding step.

### Preparation 59

### 4-(4-ethyl-piperazine-1-carbonyl)-benzoic acid

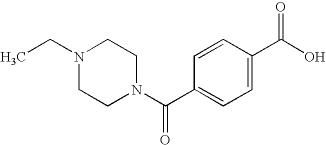

### A/ 4-(4-ethyl-piperazine-1-carbonyl)-methyl benzoate

A mixture of N-ethylpiperazine (2 eq) and mono-methyl terephtalate (4.7 g) in DMF (100 ml) is stirred at AT for 15 h, in the presence of ECDI (1.08 eq), HOBT (1.08 eq) and DIEA (2 eq). After evaporating to dryness, the residue is redissolved in ethyl acetate, washed with an aqueous NaHCO₃ solution, and the organic layer is dried over MgSO₄, filtered and evaporated. 2.5 g of desired product are obtained.

### B/ 4-(4-ethyl-piperazine-1-carbonyl)-benzoic acid

1.5 g of desired product are obtained by following General Procedure A to treat the compound obtained in the preceding step.

### Preparation 60

### 4-[ethyl-(3-piperidin-1-yl-propyl)-amino]-benzoic acid

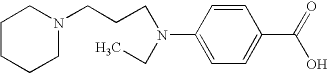

### A/ Piperidino-propyl-1-ethylamine

Following the procedure described by Watanabe, Chem. Pharm. Bull., 1997, 45 (6) pp 996-1007, 5 g of 3-piperidinopropylamine are treated with acetic anhydride in the presence of pyridine. 3.66 g of acetylated amine are obtained in the form of a yellow oil. This acetylated derivative is reduced with LAH (3 eq) in THF heating to 80° C. After treatment, 2 g of desired product are isolated in the form of a very liquid pink oil.

### B/ 4-[ethyl-(3-piperidin-1-yl-propyl)-amino]-benzonitrile

1.7 g of compound obtained in the preceding step are placed in solution in anhydrous DMSO (25 ml), then TEA (6 ml) and 4-fluorobenzonitrile (4 eq) are added and heated to 150° C. for 5 h, after which the reaction medium is poured into water. The product of the aqueous phase is extracted with diethyl ether, the organic layer dried over MgSO₄, a solution of HCl in diethyl ether is added, and the precipitate formed is collected and dried. 1.95 g of desired product are obtained in the form of a pink powder.

### C/ 4-[ethyl-(3-piperidin-1-yl-propyl)-amino]-benzoic acid

1.5 g of desired product are isolated in the form of a white powder by following General Procedure B to treat the compound obtained in the preceding step.

### Preparation 61

### 4-[1,4′]BiPiperidinyl-1′-yl-benzoic acid

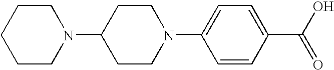

### A/ 4-[1,4′]BiPiperidinyl-1′-yl-ethyl benzoate

A mixture of ethylfluorobenzoate (6.3 g), of 4-piperidinopiperidine (1.3 eq) and K₂CO₃ (1 eq) in DMF (80 ml) is heated at 90° C. for 12 h, the solvent evaporated in vacuo, the residue redissolved in DCM, washed with water, and the organic layer is dried over MgSO₄, filtered and concentrated. 1 g of desired product are isolated after chromatography on silica eluting with a DCM/MeOH mixture (95:5 v/v/v).

### B/4-[1,4′]BiPiperidinyl-1′-yl-benzoic acid

900 mg of desired product are obtained after following General Procedure A to treat the compound obtained in the preceding step.

### Preparation 62

### 1-[4-(2-Amino-thiazol-5-yloxy)-phenyl]-3-(1-ethyl-propyl)-urea

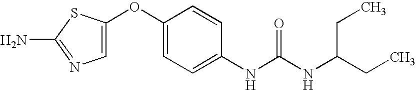

### A/ 5-(4-Nitro-phenoxy)-thiazol-2-ylamine

1.2 g of desired product are obtained by condensing 4-nitrophenol on 4 g of 2-amino-5-bromothiazole, following General Procedure P1.

### B/ [5-(4-Nitro-phenoxy)-thiazol-2-yl]-tertbutyl carbamate

Following General Procedure F, 1.3 g of the desired product are provided by reacting 4 g of compound obtained such as described in the preceding step with BOC₂O.

### C/ [5-(4-Amino-phenoxy)-thiazol-2-yl]-tertbutyl carbamate

Following General Procedure E, 1 g of desired product is obtained by hydrogenating the compound obtained in the preceding step.

### D/ (5-{4-[3-(1-Ethyl-propyl)-ureido]-phenoxy}-thiazol-2-yl)-tertbutyl carbamate

Following General Procedure H, 0.95 g of desired product are obtained from the compound obtained in the preceding step.

### E/ 1-[4-(2-Amino-thiazol-5-yloxy)-phenyl]-3-(1-ethyl-propyl)-urea

Following General Procedure C, 0.448 g of desired product are obtained from the obtained in the preceding step.

### Preparation 63

### 1-[4-(2-Amino-thiazol-yloxy)-3-methoxymethyl-phenyl]-3-(1-ethyl-propyl)-urea

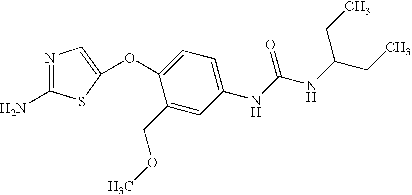

### A/ 2-Methoxymethyl-4-nitro-phenol

To a solution of Na (4 eq) in 100 ml of MeOH is added dropwise 2-chloromethyl nitrophenol (19 g) in MeOH (60 ml), stirred for 3 g at AT, then evaporated in vacuo. The residue is redissolved in water, acidified to pH 1, the aqueous layer is extracted with DCM, the organic layer is dried over MgSO₄, filtered and evaporated in vacuo. The residue is redissolved in diisopropyl ether, and the precipitate filtered and dried. 8.2 g of desired product are obtained in the form of a yellow powder.

### B/ 5-(2-Methoxymethyl-4-nitro-phenoxy)-thiazol-2-ylamine

5.1 g desired product are obtained by following General Procedure P1 to condense the compound obtained in the preceding step on 18.4 g of 2-amino-5-bromothiazole.

### C/ 5-(4-Amino-2-methoxymethyl-phenoxy)-thiazol-2-ylamine

The desired product is obtained by hydrogenating the compound of the preceding step, following General Procedure E. This product is used as such, without isolating it from the hydrogenation reaction medium.

### D/ 1-[4-(2-Amino-thiazol-5-yloxy)-3-methoxymethyl-phenyl]-3-(1-ethyl-propyl)-urea

Following General Procedure H, 4 g of desired product are obtained from the compound of the preceding step.

### Preparation 64

### 1-(1-Ethyl-propyl)-3-[3-methoxymethyl-4-(2-methylamino-thiazol-5-yloxy)-phenyl]-urea

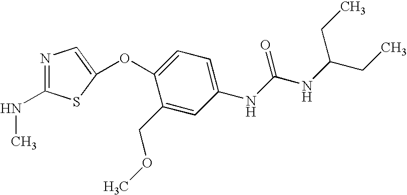

### A/ N-(5-{4-[3-(1-Ethyl-propyl)-ureido]-2-methoxymethyl-phenoxy}-thiazol-2-yl)-formamide

To 1.04 g of compound obtained under Preparation 63, in solution in THF (3.5 ml), is added a mixture of CDI (4 eq) and formic acid (4 eq) in THF (3.8 ml) and the reaction medium is stirred 48 h at AT. The solvent is evaporated, the residue redissolved in an aqueous 1 N HCl solution, extracted with ethyl acetate, the organic layer is washed with water then with an aqueous NaHCO₃ solution, dried over MgSO₄, filtered and evaporated in vacuo. 730 mg of desired product are obtained, which is used as such.

### B/ 1-(1-Ethyl-propyl)-3-[3-methoxymethyl-4-(2-methylamino-thiazol-5-yloxy)-phenyl]-urea

To a suspension of LAH (2 eq) in THF (7 ml) is added dropwise the compound obtained in the preceding step in solution in THF (8 ml). The mixture is heated at 80° C. for 24 h, returned to AT, then a few drops of a saturated aqueous Na₂SO₄ solution are added, the organic layer is dried over MgSO₄, filtered, evaporated to dryness and the residue precipitated with diethyl ether. 488 mg of desired product are obtained and used as such.

### Preparation 65

### 2-{2-(2-Amino-thiazol-5-yloxy)-5-[3-(1-ethyl-propyl)-ureido]-phenyl}-N-methyl-acetamide

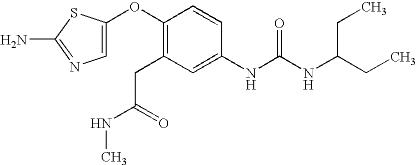

### A/ (2-Hydroxy-5-nitro-phenyl)-acetic acid

To a solution of 2-hydroxyphenylacetic acid (101 g) in water (300 ml), is slowly added nitric acid (134 ml of a 40% solution) at 0° C., the mixture stirred for 3 h keeping the temperature to between −10° C. and 0° C., then at AT for 50 h. The reaction medium is poured onto a water/ice mixture, the insoluble is filtered and the filtrate evaporated in vacuo. 26 g of desired product are isolated after chromatography of the residue on silica, eluting with a DCM/MeOH/acetic acid mixture (95:5:1 v/v/v).

### B/ (2-Hydroxy-5-nitro-phenyl)-methyl acetate

Thionyl chloride (5.4 eq) is added dropwise to a solution in MeOH (500 ml) of 21 g of compound obtained in the preceding step, stirred 2 h at AT then evaporated in vacuo. The residue is redissolved in ethyl acetate, washed with an aqueous NaHCO₃, solution, then with water and finally with 1 N aqueous solution of HCl, and the organic layer is dried over MgSO₄, filtered and evaporated in vacuo. 19.8 g of desired product are obtained which is used as such.

### C/ 2-(2-Hydroxy-5-nitro-phenyl)-N-methyl-acetamide

9 g of compound obtained in the preceding step are added to an aqueous 40% solution of methylamine (200 ml), stirred 3 h at AT then evaporated in vacuo. The residue is redissolved in water, the aqueous phase is acidified, extracted with TBME, and the organic layer is dried over MgSO₄, filtered and evaporated in vacuo. 8.9 g of desired product are obtained and used as such.

### D/ 2-[2-(2-Amino-thiazol-5-yloxy)₅-nitro-phenyl]-N-methyl-acetamide

Following General Procedure P2, using anhydrous acetone as reaction solvent, the compound obtained in the preceding step is condensed on 7.6 g of 2-amino-5-bromothiazole. 4.5 g of desired product are isolated after chromatography on silica eluting with a DCM/MeOH mixture (90:10 v/v).

### E/ 2-[5-Amino-2-(2-amino-thiazol-5-yloxy)-phenyl]-N-methyl-acetamide

The desired product is obtained by hydrogenation of 2 g of compound of the preceding step, following General Procedure E. After filtering the catalyst the solvent is partly concentrated. This product is used in solution as such, without isolating it from the hydrogenation reaction medium.

### F/ 2-{2-(2-Amino-thiazol-5-yloxy)-5-[3-(1-ethyl-propyl)-ureido]-phenyl}-N-methyl-acetamide

The compound obtained in the preceding step is treated following General Procedure H. 2 g of desired product are isolated after chromatography on silica, eluting with a DCM/MeOH/NH₄OH mixture (90:10:0.1).

### Preparation 66

### 2-[2-(2-Amino-thiazol-5-yloxy)-5-(3-dimethylamino-ureido)-phenyl]-N-methyl-acetamide

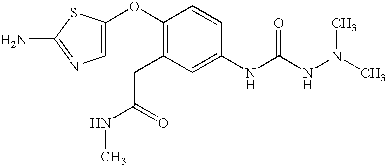

### A/ 2-[5-Amino-2-(2-amino-thiazol-5-yloxy)-phenyl]-N-methyl-acetamide

Following General Procedure E, the desired product is obtained by hydrogenating 2.1 g of compound obtained such as described under Preparation 65, step D. This product is used in solution as such without isolating it from the hydrogenation reaction medium.

### B/ 2-[2-(2-Amino-thiazol-5-yloxy)-5-(3-dimethylamino-ureido)-phenyl]-N-methyl-acetamide

A suspension of 3.5 g of CDI in THF (15 ml) is held at 0° C., the solution of compound obtained in the preceding step is added and stirred 1 h at 0° C. This mixture is cooled to −10° C., 3 ml of N,N-dimethylhydrazine are added in small portions and the mixture left to return to AT, followed by stirring at AT for 15 h and evaporation in vacuo. 1.2 g of desired product are isolated after chromatography on silica, eluting with a DCM/MeOH/NH₄OH mixture (90:10:0.1).

### Preparation 67

### 1-[4-(2-Amino-thiazol-5-yloxy)-3-fluoro-phenyl]-3-(1-ethyl-propyl)-urea

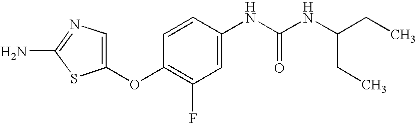

### A/ 5-(2-Fluoro-4-nitro-phenoxy)-thiazol-2-ylamine

1.7 g of desired product are obtained by condensing 2-fluoro-4-nitrophenol on 5.7 g of 2-amino-5-bromothiazole, following General Procedure P1.

### B/ 5-(4-Amino-2-fluoro-phenoxy)-thiazol-2-ylamine

Following General Procedure E, 1.4 g of desired product are obtained by hydrogenating the compound of the preceding step.

### C/ 1-[4-(2-Amino-thiazol-5-yloxy)-3-fluoro-phenyl]-3-(1-ethyl-propyl)-urea

Following General Procedure H, 0.59 g of desired product are isolated from the compound of the preceding step.

### Preparation 68

### 1-[4-(2-Amino-thiazol-5-yloxy)-3-ethoxymethyl-phenyl]-3-(1-ethyl-propyl)-urea

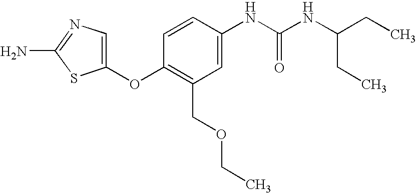

### A/ 2-Ethoxymethyl-4-nitro-phenol

To a solution of 9.4 g Na (4 eq) in absolute ethanol (220 ml), is added dropwise 2-hydroxy-5-nitrobenzyl bromide (25 g) in absolute ethanol (110 ml) and stirred 48 h at AT, then evaporated in vacuo. The residue is redissolved in water, acidified to pH 1, the precipitate formed is filtered, rinsed with water and with pentane. 20 g of desired product are obtained in the form of a black powder, which is used as such.

### B/ 5-(2-Ethoxymethyl-4-nitro-phenoxy)-thiazol-2-ylamine

6 g of desired product are obtained by condensing the compound obtained in the preceding step on 15 g of 2-amino-5-bromothiazole, following General Procedure P1.

### C/ 5-(4-Amino-2-ethoxymethyl-phenoxy)-thiazol-2-ylamine

The desired product is obtained by hydrogenating the compound obtained in the preceding step, following General Procedure E. This product is used as such, without isolating it from the hydrogenation reaction medium.

### D/ 1-[4-(2-Amino-thiazol-5-yloxy)-3-ethoxymethyl-phenyl]-3-(1-ethyl-propyl)-urea

Following General Procedure H, 3.5 g of desired product are obtained from the compound of the preceding step, after purifying the reaction medium by chromatography on silica, eluting with a DCM/MeOH/NH₄OH mixture (90:10:1v/v/v), followed by crystallization in diisopropyl ether.

### Preparation 69

### 1-[4-(2-Amino-thiazol-5-yloxy)-3-methoxy-phenyl]-3-(1-ethyl-propyl)-urea

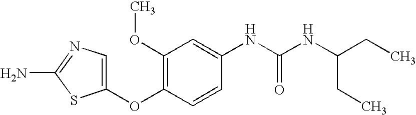

### A/ 5-(2-Methoxy-4-nitro-phenoxy)-thiazol-2-ylamine

Following General Procedure P2, 7.8 g of 4-nitroguaiacol are reacted with 10 g of 2-amino-5-bromothiazole. 2.6 g of desired product are isolated after chromatography on silica, eluting with an ethyl acetate/pentane mixture (100:30 v/v).

### B/ 5-(4-Amino-2-methoxy-phenoxy)-thiazol-2-ylamine

Following General Procedure E, 1.3 g of desired product are isolated from 1.56 g of compound obtained such as described in the preceding step.

### C/ 1-[4-(2-Amino-thiazol-5-yloxy)-3-methoxy-phenyl]-3-(1-ethyl-propyl)-urea

The compound of the preceding step is reacted in accordance with General Procedure H. 1 g of desired product is isolated after chromatography on silica, eluting with a DCM/MeOH/NH₄OH mixture (95:5:0.5 v/v/v) followed by precipitation in diethyl ether.

### Preparation 70

### 1-[4-(4-Amino-phenoxy)-3-methoxy-phenyl]-3-(1-ethyl-propyl)-urea

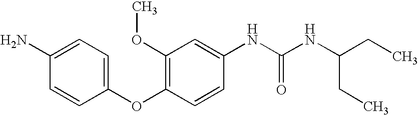

### Method I

### A/ (4-Hydroxy-3-methoxy-phenyl)-tertbutyl carbamate

14 g of desired product are isolated after hydrogenating 4-nitroguaiacol (10 g), following General Procedure E, followed by protection with a BOC group of the aniline thus obtained in accordance with General Procedure F.

### B/ [3-Methoxy-4-(4-nitro-phenoxy)-phenyl]-tertbutyl carbamate

Following General Procedure 0, 9.8 g of compound obtained such as described in the preceding step are condensed on 5.8 g of 1-chloronitrobenzene. 8.8 g of desired product are obtained after chromatography on silica, eluting with a cyclohexane/ethyl acetate mixture (80:20 v/v).

### C/ 3-Methoxy 4-nitro-phenoxy)-phenylamine

The compound of the preceding step is treated in accordance with General Procedure C. 3.5 g of desired product are isolated in the form of a free base, after chromatography on silica, eluting with a cyclohexane/ethyl acetate mixture (60:40 v/v).

### D/ 1-(1-Ethyl-propyl)-3-[3-methoxyl-4-nitro-phenoxy)-phenyl]-urea

5.9 g of compound obtained such as described in the preceding step are treated following General Procedure H. 2.9 g of desired product are isolated after chromatography on silica, eluting with a cyclohexane/ethyl acetate mixture (70:30 v/v).

### E/ 1-[4-(4 Amino-phenoxy)-3-methoxy-phenyl]-3-(1-ethyl-propyl)-urea

1.95 g of desired product are obtained from the compound of the preceding step, following General Procedure E.

### Method II

### A/ [4-(2-Methoxy-4-nitro-phenoxy)-phenyl]-tertbutyl carbamate

29.8 g of desired product are obtained by condensing 4-N—BOC-aminophenol on 2-chloro-5-nitroanisole (30 g), following General Procedure O.

### B/ [4-(4-Amino-2-methoxy-phenoxy)-phenyl]-tertbutyl carbamate

22 g of desired product are obtained from the compound of the preceding step, following General Procedure E.

### C/ (4-{4-[3-(1-ethyl-propyl)-ureido]-2-methoxy-phenoxy}-phenyl)-tertbutyl carbamate

29 g of desired product are obtained from the compound of the preceding step, following General Procedure H.

### D/ 1-[4-(4-Amino-phenoxy)-3-methoxy-phenyl]-3-(1-ethyl-propyl)-urea

The compound of the preceding step is treated according to General Procedure C. 3.4 g of desired product are obtained in the form of a free base, after chromatography on silica, eluting with a DCM/MeOH/NH₄OH mixture (95:5:0.5 v/v/v).

### Preparation 71

### 1-[4-(4-Amino-phenoxy)-3-ethoxy-phenyl]-3-(1-ethyl-propyl)-urea

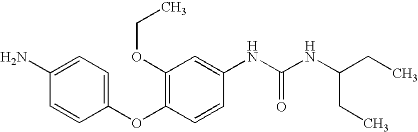

### A/ 1,2-Diethoxy-4-nitro-benzene

NaH (2.2 eq) is added to a solution of nitrocatechol (20 g) in DMF (550 ml), heated 45 min at 50° C., then iodoethane (2.2 eq) is added and heated for 2 h at 50° C. The reaction medium is poured into water, the precipitate filtered, redissolved in TBME, this organic layer is washed with water, dried over MgSO₄, the solid obtained is filtered, evaporated and washed with pentane. 18 g of desired product are obtained and used as such.

### B/ 2-Ethoxy-4-nitro-phenol

The product obtained in the preceding step is heated under reflux 18 h in a mixture of water/methoxyethanol (150 ml/100 ml), in the presence of KOH (10 eq). The precipitate is filtered, redissolved in water, acidified to pH 1 with concentrated HCl. The aqueous layer is extracted with TBME, the organic layer washed with water, dried over MgSO₄, filtered and concentrated dry. 13.9 g of desired product are obtained and used as such.

### C/ (3-Ethoxy-4-hydroxy-phenyl)-tertbuyl carbamate

2.45 g of desired product are isolated after following General Procedure E to hydrogenate 2.1 g of compound obtained in the preceding step, followed by protection of the aniline thus obtained by a BOC group in accordance with General Procedure F.

### D/ [3-Ethoxy-4-(4-nitro-phenoxy)-phenyl]-tertbutyl carbamate

Following General Procedure O, 2.35 g of compound of the preceding step are condensed on 1.31 g of 1-fluoro-4-nitrobenzene. 0.80 g of desired product are isolated after chromatography on silica, eluting with a cyclohexane/ethyl acetate mixture (85:15 v/v).

### E/ 3-Ethoxy-4-(4-nitro-phenoxy)-phenylamine

0.98 g of desired product are obtained in the form of a TFA salt from the compound of the preceding step, following General Procedure C.

### F/ 1-[3-Ethoxy-4-(4-nitro-phenoxy)-phenyl]-3-(1-ethyl-propyl)-urea

The compound of the preceding step is treated according to General Procedure H. 0.3 g of desired product are isolated after chromatography on silica, eluting with DCM and then with a cyclohexane/ethyl acetate mixture (75:25 v/v).

### G/ 1-[4-(4-Amino-phenoxy)-3-ethoxy-phenyl]-3-(1-ethyl-propyl)-urea

0.23 g of desired product are obtained from the compound of the preceding step, following General Procedure E.

### Preparation 72

### 1-[4-(4-Amino-phenoxy)-3-methyl-phenyl]-3-(1-ethyl-propyl)-urea

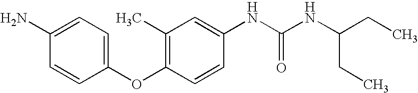

### A/ (4-Hydroxy-3-methyl-phenyl)-tertbutyl carbamate

16 g of desired product are isolated by following General Procedure E to hydrogenate 10.9 g of 2-methyl-4-nitrophenol, followed by protection of the aniline thus obtained by a BOC group in accordance with General Procedure F.

### B/ [3-Methyl-4-(4-nitro-phenoxy)-phenyl]-tertbutyl carbamate

20.5 g of desired product are obtained by condensing the compound of the preceding step on 4-fluoro-nitrobenzene (13.5 g), following General Procedure O.

### C/ 3-Methyl-4-(4-nitro-phenoxy)-phenylamine

9 g of desired product are obtained in the form of a TFA salt from 8.6 g of compound of the preceding step, following General Procedure C.

### D/ 1-(1-Ethyl-propyl)-3-[3-methyl-4-(4-nitro-phenoxy)-phenyl]-urea

6.4 g of desired product are obtained from the compound of the preceding step, following General Procedure H.

### E/ -[4-(4-Amino-phenoxy)-3-methyl-phenyl]-3-(1-ethyl-propyl)-urea

6 g of desired product are obtained from the compound of the preceding step, following General Procedure E.

### Preparation 73

### 1-[4-(4-Amino-2-methoxymethyl-phenoxy)-phenyl]-3-(1-ethyl-propyl)-urea

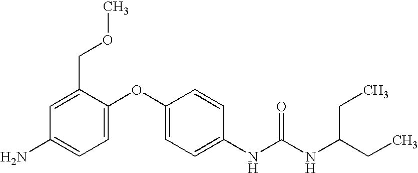

### A/ (4-Hydroxy-3-methoxymethyl-phenyl)-tertbutyl carbamate

7.6 g of desired product are isolated in the form of yellow crystals after using General Procedure E to hydrogenate 13.6 g of compound obtained such as described under Preparation 63, step A, followed by protection of the aniline thus obtained by a BOC group in accordance with General Procedure F.

### B/ [3-Methoxymethyl-4-(4-nitro-phenoxy)-phenyl]-tertbutyl carbamate

9.6 g of desired product are obtained by condensing the compound of the preceding step on 4-fluoronitrobenzene, following General Procedure O.

### C/ [4-(4-Amino-phenoxy)-3-methoxymethyl-phenyl]-tertbutyl carbamate

Following General Procedure E, 12 g of desired product are obtained from 14.8 g of compound obtained such as described in the preceding step.

### D/ (4-{4-[3-(1-Ethyl-propyl)-ureido]-phenoxy}-3-methoxymethyl-phenyl)-tertbutyl carbamate

6 g of compound of the preceding step are treated following General Procedure H. 6.74 g of desired product are isolated after chromatography on silica, eluting with a cyclohexane/ethyl acetate mixture (70:30 v/v).

### E/ 1-[4-(4-Amino-2-methoxymethyl-phenoxy)-phenyl]-3-(1-ethyl-propyl)-urea

The compound of the preceding step is treated following General Procedure C. The solvent is evaporated in vacuo, the residue redissolved in base water, the product extracted with DCM, and the organic layer is evaporated. 4.28 g of desired product are isolated in hydrochloride form by treating the residue obtained with HCl in isopropanol, followed by evaporation to dryness and washing the solid with pentane and TBME.

### Preparation 74

### 1-[4-(4-Amino-2-methoxy-phenoxy)-phenyl]-3-(1-ethyl-propyl)-urea

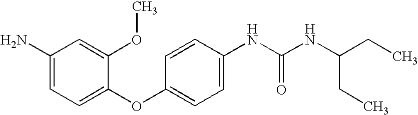

### A/ [4-(4-Amino-phenoxy)-3-methoxy-phenyl]-tertbutyl carbamate

Following General Procedure E, 4 g of desired product are obtained from 4.4 g of compound obtained such as described under Preparation 70, Method I, step B.

### B/ (4-{4-[3-(1-Ethyl-propyl)-ureido]-phenoxy}-3-methoxy-phenyl)-tertbutyl carbamate

Following General Procedure H, 3 g of desired product are obtained from the compound of the preceding step.

### C/ 1-[4-(4-Amino-2-methoxy-phenoxy)-phenyl]-3-(1-ethyl-propyl)-urea

Using General Procedure C, 3.5 g of desired product are obtained in the form of a TFA salt from the compound of the preceding step.

### Preparation 75

### 1-[4-(4-Amino-2-methyl-phenoxy)-phenyl]-3-(1-ethyl-propyl)-urea

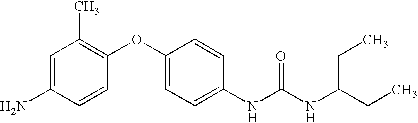

### A/ [4-(2-Methyl-4-nitro-phenoxy)-phenyl]-tertbutyl carbamate

Following General Procedure O, 8.3 g of desired product are obtained in the form of a pale yellow powder, by condensing 4-N—BOC-aminophenol on 2-chloro-5-nitrotoluene (10 g).

### B/ 4-(2-Methyl-4-nitro-phenoxy)-phenylamine

Following General Procedure C, 8.6 g of desired product are obtained from the compound of the preceding step. This compound is used as such for the following step.

### C/ 1-(1-Ethyl-propyl)-3-[4-(2-methyl-4-nitro-phenoxy)-phenyl]-urea

Following General Procedure H, 2.2 g of desired product are obtained from 3 g of the compound obtained in the preceding step.

### D/ 1-[4-(4-Amino-2-methyl-phenoxy)-phenyl]-3-(1-ethyl-propyl)-urea

Following General Procedure E, 2.3 g of desired product are obtained from the compound of the preceding step.

### Preparation 76

### 1-[4-(4-Amino-2-methyl-phenoxy)-3-methoxy-phenyl]-3-(1-ethyl-propyl)-urea

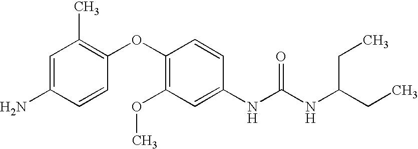

### A/ [3-Methoxy-4-(2-methyl-4-nitro-phenoxy)-phenyl]-tertbutyl carbamate

3.56 g of compound obtained such as described under Preparation 70, Method I, step A are condensed on 2.31 g of 2-fluoro-5-nitrotoluene, following General Procedure O. 3.36 g of desired product are isolated after chromatography on silica, eluting with a DCM/cyclohexane mixture (20:10 v/v).

### B/ 3-Methoxy-4-(2-methyl-4-nitro-phenoxy)-phenylamine

Following General Procedure C, 5 g of desired product are obtained in the form of a TFA salt, from 4.73 g of compound obtained such as described in the preceding step.

### C/ 1-(1-Ethyl-propyl)-3-[3-methoxy-4-(2-methyl-4-nitro-phenoxy)-phenyl]-urea

General Procedure H is followed to treat the compound obtained in the preceding step. 5.6 g of desired product are isolated after chromatography on silica, eluting with a DCM/MeOH mixture (99:1 v/v).

### D/ 1-[4-(4-Amino-2-methyl-phenoxy)-3-methoxy-phenyl]-3-(1-ethyl-propyl)-urea

The compound of the preceding step is treated following General Procedure E. The product obtained is then dissolved in DCM, precipitated with concentrated HCl, the precipitate collected, dissolved in a minimum quantity of MeOH and again precipitated in a DCM/diethyl ether mixture. 3.15 g of desired product are isolated in hydrochloride form.

### Preparation 77

### 2-(4-Amino-phenoxy)-5-(3-isopropyl-ureido)-methyl benzoate

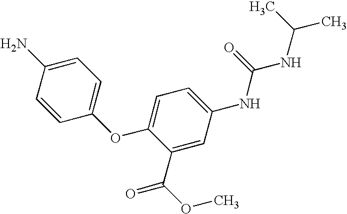

### A/ 2-Chloro-5-nitro-methyl benzoate

A mixture of 2-chloro-5-nitrobenzoic acid (35 g), DMF (1 ml) and SOCl₂ (430 ml) is heated under reflux for 2 h, concentrated in vacuo and added to the residue of MeOH keeping the temperature at 0° C. After stirring 18 h at AT and evaporation in vacuo, the residue is redissolved in DCM, the organic layer is washed with an aqueous NaOH solution then with an aqueous NaCl solution, and the organic layer is dried over MgSO₄, filtered and evaporated to dryness. 37 g of desired product are obtained and used as such.

### B/ 2-(4-tert-Butoxycarbonylamino-phenoxy)-5-nitro-methyl benzoate

Following General Procedure O, 38 g of desired product are obtained in the form of an orange powder, by condensing 4-N—BOC-aminophenol on the compound obtained in the preceding step.

### C/ 5-Amino-2-(4-tert-butoxycarbonylamino-phenoxy)-methyl benzoate

Following General Procedure D, 3.9 g of desired product are obtained from 7 g of compound obtained in the preceding step.

### D/ 2-(4-tert-Butoxycarbonylamino-phenoxy)-5-[3-isopropyl-ureido]-methyl benzoate

Following General Procedure N, 2.7 g of desired product are obtained from 2.5 g of compound obtained in the preceding step.

### E/ 2-(4-Amino-phenoxy)-5-(3-isopropyl-ureido)-methyl benzoate

Following General Procedure C, 2.1 g of desired product are obtained from the compound of the preceding step.

### Preparation 78

### 1-(1-Ethyl-propyl)-3-{3-methoxy-4-[4-(2,2,2-trifluoro-ethylamino)-phenoxy]-phenyl}-urea

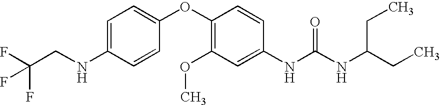

### A/ N-(4-{4-[3-(1-Ethyl-propyl)-ureido]-2-methoxy-phenoxy}-phenyl)-2,2,2-trifluoro-acetamide

To a solution of compound obtained such as described under Preparation 70 (1 g) in TFA (3 ml), is added trifluoroacetic anhydride (3 ml) and stirred 30 min at AT, then the reaction medium is poured into water. The precipitate formed is filtered, rinsed with water and dried. 1.08 g of desired product are obtained in the form of a white powder.

### B/ 1-(1-Ethyl-propyl)-3-{3-methoxy-[4-(2,2,2-trifluoro-ethylamino)-phenoxy]-phenyl}-urea

To a suspension of LAH (3 eq) in THF (10 ml) heated to 60° C., the compound obtained in the preceding step is added, the mixture heated at 60° C. for 1 h followed by the addition of an aqueous Na₂SO₄ solution and filtering. The filtrate is evaporated and the residue washed in diethyl ether. 620 mg of desired product are obtained and used as such.

### Preparation 79

### 1-[4-(4-Amino-phenoxy)-3-propoxy-phenyl]-3-(1-ethyl-propyl)-urea

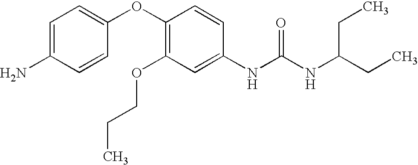

### A/ 4-Nitro-1,2-dipropoxy-benzene

To a solution of nitrocatechol (25 g) in DMF (400 ml) is added NaH (2.2 eq) keeping the temperature close to AT, then iodopropane (42 ml) is added and heated 2 h at 50° C., the reaction medium is poured into water and the precipitate formed is filtered and washed with water. The precipitate is solubilized in diethyl ether, dried over MgSO₄ and evaporated in vacuo. 32 g of desired product are isolated after chromatography on silica, eluting with a cyclohexane/ethyl acetate mixture (95:5 v/v).

### B/ 4-Nitro-2-propoxy-phenol

The product obtained in the preceding step is heated under reflux for 48 h in a mixture of water/methoxyethanol (275 ml/175 ml). Part of the solvents are concentrated in vacuo, decanted for 15 h, and the precipitate is filtered and rinsed with TBME. The solid obtained is redissolved in water, acidified with concentrated HCl, the product is extracted with TBME and the organic layer is dried ovrt MgSO₄, filtered and evaporated in vacuo. 19 g of desired product are obtained in the form of a beige solid.

### C/ (4-Hydroxy-3-propoxy-phenyl)-tertbutyl carbamate

13 g of desired product are isolated after following General Procedure E to hydrogenate the compound obtained in the preceding step, followed by protection of the aniline thus obtained with a BOC group in accordance with General Procedure F.

### D/ [4-(4-Nitrophenoxy)-3-propoxy-phenyl]-tertbutyl carbamate

Following General Procedure O, 15.6 g of desired product are obtained by condensing the compound obtained in the preceding step on 7.5 g 1-fluoro-nitrobenzene.

### E/ 4-(4-Nitro-phenoxy)-3-propoxy-phenylamine

Following General Procedure C, 11.5 g of desired product are obtained from the compound of the preceding step.

### F/ 1-(1-Ethyl-propyl)-3-[4-(4-nitro-phenoxy)-3-propoxy-phenyl]-urea

Following General Procedure H, 5.2 g of desired product are obtained from 5.8 g of compound of the preceding step.

### G/ 1-[4-(4-Amino-phenoxy) 3 propoxy-phenyl]-3-(1-ethyl-propyl)-urea

Following General Procedure E, 2.7 g of desired product are obtained from the compound of the preceding step.

### Preparation 80

### 1-[4-(4-Amino-phenoxy)-3-chloro-phenyl]-3-(1-ethyl-propyl)-urea

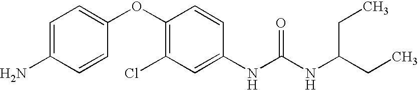

### A/ (3-Chloro-4-hydroxy-phenyl)-tertbutyl carbamate

Following General Procedure F, 12 g of desired product are obtained from 5.1 g of 2-chloroaminophenol.

### B/ [3-Chloro-4-(4-nitro-phenoxy)-phenyl]-tertbutyl carbamate

Following General Procedure O, 9.8 g of desired product are obtained by condensing the compound obtained in the preceding step on 4-fluoronitrobenzene.

### C/ 3-Chloro-4-(4-nitro-phenoxy)-phenylamine

Following General Procedure C, 4.6 g of desired product are obtained in the form of a free base, from 6.8 g of compound obtained in the preceding step.

### D/ 1-[3-Chloro-4-(4-nitro-phenoxy)-phenyl]-3-(1-ethyl-propyl)-urea

Following General Procedure H, 2.2 g of desired product are obtained from the compound of the preceding step.

### E/ 1-[4-(4-Amino-phenoxychloro-phenyl]-3-(1-ethyl-propyl)-urea

The compound of the preceding step is reduced by catalytic hydrogenation in ethyl acetate (100 ml) in the presence of 0.5 g of 5% sulfided platinum on charcoal for 4 h at 50° C., the catalyst is filtered and the filtrate evaporated. 2 g of desired product are obtained, and used as such.

### Preparation 81

### 1-[4-(4-Amino-phenoxy)-3-methoxymethyl-phenyl]-3-(1-ethyl-propyl)-urea

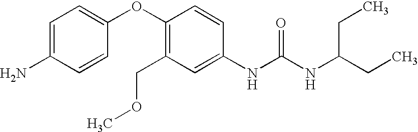

### A/ 3-Methoxymethyl-4-(4-nitro-phenoxy)-phenylamine

Following General Procedure C, 6.7 g of desired product are obtained from 9.6 g of compound obtained such as described under Preparation 73, step B.

### B/ 1-(1-Ethyl-propyl)-3-[3-methoxymethyl-4-(4-nitro-phenoxy)-phenyl]-urea

Following General Procedure H, 4.2 g of desired product are obtained from 3 g of compound of the preceding step.

### C/ 1-[4-(4-Amino-phenoxy)-3-methoxymethyl-phenyl]-3-(1-ethyl-propyl)-urea

Following General Procedure E, 3.9 g of desired product are obtained from the compound of the preceding step.

### Preparation 82

### 1-[4-(4-Amino-phenoxy)-phenyl]-3-(1-ethyl-propyl)-urea

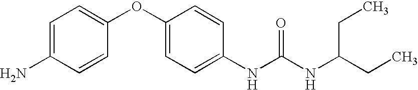

### A/ [4-(4-Nitro-phenoxy)-phenyl]-tertbutyl carbamate

Following General Procedure O, 10.86 g of desired product are obtained by condensing 4-N—BOC-aminophenol on 14.7 g of 4-chloronitrobenzene.

### B/ 4-(4-Nitro-phenoxy)-phenylamine

Following General Procedure C, 7.29 g of desired product are obtained from the compound of the preceding step.

### C/ 1-(1-Ethyl-propyl-3-[4-(4-nitro-phenoxy)-phenyl]-urea

Following General Procedure H, 4.1 g of desired product are obtained from 4 g of compound obtained in the preceding step.

### D/ 1-[4-(4-Amino phenoxy)-phenyl]-3-(1-ethyl-propylurea

Following General Procedure E, 3.4 g of desired product are obtained from the compound obtained in the preceding step.

### Preparation 83

### 1-[4-(4-Amino-phenoxy)-3-fluoro-phenyl]-3-(1-ethyl-propyl)-urea

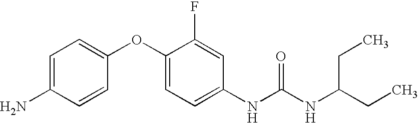

### A/ 2-Fluoro-4-N—BOC-aminophenol

4 g of 2-fluoro-4-nitrophenol are stirred in a hydrogen atmosphere, in the presence of 10% palladium on charcoal (1.2 g) and (BOC)₂O (1.05 eq), in 120 ml of THF for 11 h, the catalyst is filtered and the filtrate is concentrated to dryness. 5.89 g of desired product are isolated in the form of a white powder after precipitating the residue with pentane.

### B/ [3-Fluoro-4-(4-nitro-phenoxy)-phenyl]-tertbutyl carbamate

Following General Procedure O, 12.08 g of compound obtained such as described in the preceding step are condensed on 10.8 g of 4-chloronitrobenzene. 7.4 g of desired product are isolated after chromatography on silica, eluting with a cyclohexane/ethyl acetate mixture (90:10 v/v) followed by precipitation in pentane.

### C/ 3-Fluoro-4-(4-nitro-phenoxy)-phenylamine

Following General Procedure C, 4.8 g of desired product are obtained in the form of a free base, from the compound of the preceding step.

### D/ 1-(1-Ethyl-propyl)-3-[3-fluoro-4-(4-nitro-phenoxy-phenyl]-urea

Following General Procedure H, 3.7 g of desired product are obtained from 4 g of compound obtained in the preceding step.

### E/ 1-[4-(4-Amino-phenoxy)-3-fluoro-phenyl]-3-(1-ethyl-propyl)-urea

The compound of the preceding step is treated following General Procedure E. 2.46 g of desired product are isolated in the form of an HCl salt after chromatography on silica, eluting with a cyclohexane/ethyl acetate mixture (40:60 v/v), followed by treatment with HCl in diethyl ether.

### Preparation 84

### 1-[4-(2,3-Dihydro-1H-indol-5-yloxy)-3-methoxy-phenyl]-3-(1-ethyl-propyl)-urea

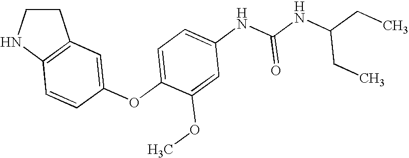

### A/ 5-Methoxy-2,3-dihydro-1H-indole

In an inert atmosphere, a solution of 5-methoxyindole (25 g) in acetic acid to which NaBH₃CN (1.5 eq) is added in portions, is stirred 15 h at AT. Water is added to the reaction medium, which is basified to pH 12 with a concentrated aqueous NaOH solution, extracted with DCM, the organic layer is washed with a saturated aqueous NaCl solution, the organic layer is dried over MgSO₄, filtered and evaporated in vacuo. 25 g of desired product is obtained, which is used as such.

### B/ 2,3-Dihydro-1H-indol-5-ol

12 g of compound obtained in the preceding step are heated at 140° C. for 3 h in HBr (48%, 200 ml). After cooling to AT, filtering, the filtrate is concentrated dry. The residue is washed with acetone and dried. 14.8 g of desired product are obtained and used as such.

### C/ 5-Hydroxy-2,3-dihydro-indole-1-tertbutyl carboxylate

Following General Procedure F, 31 g of desired product are obtained from 30.1 g of compound obtained such as described in the preceding step.

### D/ 5-(2-Methoxy-4-nitro-phenoxy)-2,3-dihydro-indole-1-tertbutyl carboxylate

Following General Procedure O, 20.5 g of desired product are obtained by condensing the compound obtained in the preceding step on 12.35 g of 2-chloro-5-nitroanisole.

### E/ 5-(4-Amino-2-methoxy-phenoxyl)-2,3-dihydro-indole-1-tertbutyl carboxylate

Powder Zn (20 eq) is added in small portions to a mixture of 5 g of product obtained in the preceding step and of NH₄Cl (2 eq) in MeOH (600 ml), followed by heating at 60° C. for 2 h, hot filtering on celite, hot washing with MeOH and concentrating the filtrate to dryness. 4.6 g of desired product are obtained, which is used as such.

### F/ 5-{4-[3-(1-Ethyl-propyl)-ureido]-2-methoxy-phenoxy}-2,3-dihydro-indole-1-tertbutyl carboxylate

Following General Procedure H, 12.6 g of desired product are obtained from 9.3 g of compound obtained such as described in the preceding step.

### G/ 1-[4-(2,3-Dihydro-1H-indol-5-yloxy)-3-methoxy-phenyl]-3-(1-ethyl-propyl)-urea

Following General Procedure C, 4.3 g of desired product are obtained from 5.2 g of compound obtained such as described in the preceding step.

### Preparation 85

### 1-[4-(4-Amino-phenoxy)-2-fluoro-phenyl]-3-(1-ethyl-propyl)-urea

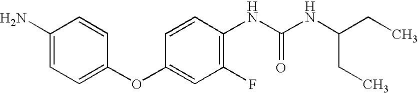

### A/ 3-Fluoro-4-N—BOC-aminophenol

3-fluoro-4-nitrophenol (23.7 g) is stirred in a hydrogen atmosphere, in the presence of 10% palladium on charcoal (7 g) and (BOC)₂O (1.05 eq) in THF for 11 h, after which the catalyst is filtered, rinsed with MeOH and the filtrate concentrated to dryness. 32 g of desired product are isolated in the form of a pink powder after filtering on silica, eluting with a cyclohexane/ethyl acetate mixture (85:15 v/v).

### B/ [2-Fluoro-4-(4-nitro-phenoxy)-phenyl]-tertbutyl carbamate

Following General Procedure O, 16 g of desired product are obtained in powder form, by condensing the compound of the preceding step on 8.6 g of 4 fluoronitrobenzene.

### C/ 2-Fluoro-4-(4-nitro-phenoxy)-phenylamine

Following General Procedure C, 8 g of desired product are obtained from the compound of the preceding step.

### D/ 1-(1-Ethyl-propyl)-3-[2-fluoro-4-(4-nitro-phenoxy)-phenyl]-urea

Following General Procedure H, 2 g of desired product are obtained from 4 g of compound obtained in the preceding step.

### E/ 1-[4-(4-Amino-phenoxy)-2-fluoro-phenyl]-3-(1-ethyl-propyl)-urea

Following General Procedure E, 1.5 g of desired product are obtained from the compound obtained in the preceding step.

### Preparation 86

### 1-[4-(4-Amino-2-methyl-phenoxy)-3-methoxymethyl-phenyl]-3-(1-ethyl-propyl)-urea

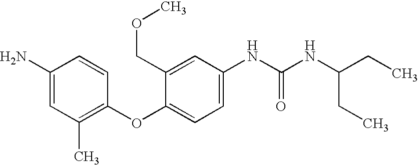

### A/ [3-Methoxymethyl-4-(2-methyl-4-nitro-phenoxy)-phenyl]-tertbutyl carbamate

Following General Procedure O, 1.8 g of desired product are obtained by condensing the compound obtained such as described in Preparation 73, step A, on 1.9 g of 2-chloro-5-nitrotoluene.

### B/ 3-Methoxymethyl-4-(2-methyl-4-nitro-phenoxy)-phenylamine

Following General Procedure C, 2.3 g of desired product are isolated from 3.6 g of compound obtained such as described in the preceding step.

### C/ 1-(1-Ethyl-propyl)-3-[3-methoxymethyl-4-(2-methyl-4-nitro-phenoxy)-phenyl]-urea

Following General Procedure H, 4.9 g of desired product are isolated from 4 g of compound obtained such as described in the preceding step.

### D/ 1-[4-(4-Amino-2-methyl-phenoxy)-3-methoxymethyl-phenyl]-3-(1-ethyl-propyl)-urea

Following General Procedure E, 2.5 g of desired product are obtained from the compound of the preceding step.

### Preparation 87

### 1-[4-(4-Amino-3-fluoro-phenoxy)-phenyl]-3-(1-ethyl-propyl)-urea

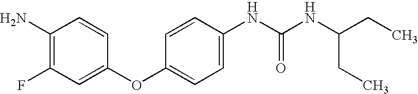

### A/ [4-(4-Amino-phenoxy)-2-fluorophenyl]-tertbutyl carbamate

Following General Procedure E, 3.6 g of desired product are obtained from 3.8 g of compound obtained such as described under Preparation 85, step B.

### B/ (4-{4-[3-(1-Ethyl-propyl)-ureido]-phenoxy}-2-fluoro-phenyl)-tertbutyl carbamate

Following General Procedure H, 6 g of desired product are obtained from 4.5 g of compound obtained such as described in the preceding step.

### C/ 1-[4-(4-Amino-3-fluoro-phenoxy)-phenyl]-3-(1-ethyl-propyl)-urea

Following General Procedure C, 4.6 g of desired product are obtained from the compound of the preceding step.

### Preparation 88

### 1-[4-(4-Amino-3-fluoro-phenoxy)-3-chloro-phenyl]-3-(1-ethyl-propyl)-urea

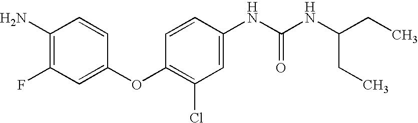

### A/ [4-(2-Chloro-4-nitro-phenoxy)-2-fluoro-phenyl]-tertbutyl carbamate

Following General Procedure O, and after crystallization in TBME, 8.9 g of desired product are obtained by condensing 7.1 g of compound obtained such as described under Preparation 85, step A, on 5.5 g of 4-fluoro-3-chloronitrobenzene.

### B/ [4-(4-Amino-2-chloro-phenoxy)-2-fluoro-phenyl]-tertbutyl carbamate

6.2 g of compound obtained such as described in the preceding step are reduced by catalytic hydrogenation in ethyl acetate (200 ml), in the presence of 5% sulfided platinum on charcoal, at AT and AP. The catalyst is filtered and the filtrate is evaporated. 6.9 g of desired product are obtained, which is used as such.

### C/ (4-{2-Chloro-4-[3-(1-ethyl-propyl)-ureido]-phenoxy}-2-fluoro-phenyl)-tertbutyl carbamate

Following General Procedure H, 7.4 g of desired product are obtained from the compound of the preceding step.

### D/ 1-[4-(4-Amino-3-fluoro-phenoxy)-3-chloro-phenyl]-3-(1-ethyl-propyl)-urea

Following General Procedure C, 7.5 g of desired product are obtained in the form of a TFA salt from the compound of the preceding step.

### Preparation 89

### 1-[4-(4-Amino-3-methoxy-phenoxy)-phenyl]-3-(1-ethyl-propyl)-urea

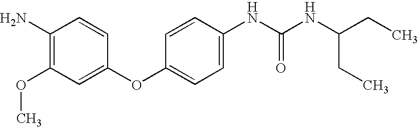

### A/ [4-(3-Methoxy-4-nitro-phenoxy)-phenyl]-tertbutyl carbamate

Following General Procedure O, 9.4 g of desired product are obtained by condensing 4-N—BOC-aminophenol on 8.97 g of 5-chloro-2-nitroanisole.

### B/ 4-(3-Methoxy-4-nitro-phenoxy)-phenylamine

Following General Procedure C, 7 g of desired product are obtained from the compound of the preceding step.

### C/ 1-(1-Ethyl-propyl-3-[4-(3-methoxy-4-nitro-phenoxy)-phenyl]-urea

Following General Procedure H, 5.6 g of desired product are obtained from the compound of the preceding step.

### D/ 1-[4-(4-Amino-3-methoxy-phenoxy)-phenyl]-3-(1-ethyl-propyl)-urea

Following General Procedure E, 4.5 g of desired product are obtained from the compound of the preceding step.

### Preparation 90

### 1-[4-(4-Amino-phenoxy)-3-ethyl-phenyl]-3-(1-ethyl-propyl)-urea

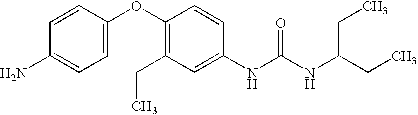

### A/ 2-Ethyl-4-nitro-phenol

To a solution of 2-ethylphenol (38 ml) in ACN (75 ml) is added 1 eq of ammonium nitrite and, after cooling to −10° C., 1.1 eq of trifluoroacetic anhydride is added dropwise, stirred at −10° C. for 1 h then poured onto ice. After evaporating the ACN in vacuo, diluting with an aqueous NaCl solution, and extracting with DCM, the organic layer is dried over MgSO₄, filtered and concentrated in vacuo. 7.3 g of desired product are isolated after chromatography of the residue on silica, eluting with a cyclohexane/ethyl acetate mixture (95:5 v/v).

### B/ (3-Ethyl-4-hydroxy-phenyl)-tertbutyl carbamate

9.8 g of desired product are isolated after following General Procedure E to hydrogenate the compound obtained in the preceding step, followed by protection of the aniline thus obtained by a BOC group in accordance with General Procedure F.

### C/ [3-Ethyl-4-(4-nitro-phenoxy)-phenyl]-tertbutyl carbamate

Following General Procedure O, 3.38 g of desired product are obtained by condensing the compound of the preceding step on 9.8 g of 4-chloronitrobenzene.

### D/ 3-Ethyl-4-(4-nitro-phenoxy)-phénylamine

Following General Procedure C, 2.2 g of desired product are obtained from the compound of the preceding step.

### E/ 1-[3-Ethyl-4-(4-nitro-phenoxy)-phenyl]-3-(1-ethyl-propyl)-urea

Following General Procedure H, 1.62 g of desired product are obtained from the compound of the preceding step.

### F/ 1-[4-(4-Amino-phenoxy)-3-ethyl-phenyl]-3-(1-ethyl-propyl)-urea

Following General Procedure E, 1.1 g of desired product are obtained from the compound of the preceding step.

### Preparation 91

### 1-[4-(4-Ethylamino-phenoxy)-3-methoxy-phenyl]-3-(1-ethyl-propyl)-urea

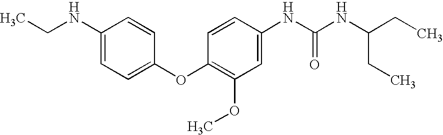

### A/ N-(4-{4-[3-(1-Ethyl-propyl)-ureido]-2-methoxy-phenoxy)-phenyl}acetamide

To a solution in acetic acid (10 ml) of compound obtained such as described under Preparation 70 (4 g), is added acetic anhydride (5 ml) which is stirred 1 h at AT. The reaction medium is poured into water, the precipitate formed is filtered, washed with water and dried. 4.2 g of desired product are obtained in the form of a white powder.

### B/ 1-[4-(4-Ethylamino-phenoxy)-3-methoxy-phenyl]-3-(1-ethyl-propyl)-urea

To a suspension of LAH (3 eq) in THF (100 ml) is added the compound obtained in the preceding step and heated at 60° C. for 1 h. Then a saturated aqueous Na₂SO₄ solution is added, the mixture is filtered, extracted with DCM, the organic layer is dried over MgSO₄, filtered and the filtrate evaporated. 2.2 g of desired product are isolated in powder form after chromatography on silica, eluting with a DCM/MeOH/NH₄OH mixture (95:5:0.05 v/v/v), followed by crystallization in TBME.

### Preparation 92

### 1-[4-(4-Amino-phenoxy)-phenyl]-3-isopropyl-urea

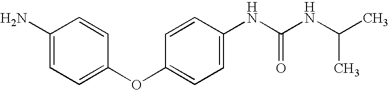

### A/ {4-[4-(3-Isopropyl-ureido)-phenoxy]-phenyl}-tertbutyl carbamate

Following General Procedure N, the desired product is obtained from 3 g of compound obtained such as described under Preparation 82, step B and from 1.7 g of isopropyl isocyanate.

### B/ 1-[4-(4-Amino-phenoxy)-phenyl]-3 isopropyl-urea

Following General Procedure C, 3.7 g of desired product are obtained in the form of a TFA salt, from the compound of the preceding step.

### Preparation 93

### 1-[4-(4-Amino-3-methoxy-phenoxy)-phenyl]-3-isopropyl-urea

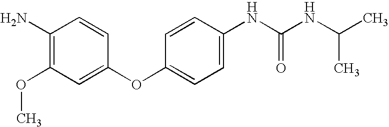

### A/ 1-Isopropyl-3-[4-(3-methoxy-4-nitro-phenoxy)-phenyl]-urea

Following General Procedure N, 1.9 g of desired product are obtained from 2 g of compound obtained such as described under Preparation 89, step B and from isopropyl isocyanate.

### B/ 1-[4-(4-Amino-3-methoxy-phenoxy)-phenyl]-3-isopropyl-urea

Following General Procedure E, 1.3 g of desired product are obtained from the compound of the preceding step.

### Preparation 94

### 1-[4-(4-Amino-phenoxy)-3-trifluoromethyl-phenyl]-3-(1-ethyl-propyl)-urea

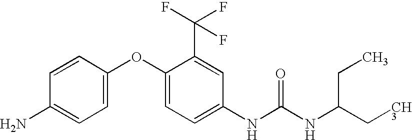

### A/ 4-Nitro-2-trifluoromethyl-phenol

20 g of 2-trifluoromethyl-4-nitroanisole are added to 100 g of pyridine hydrochloride, heated to 140° C., and the mixture heated for 2 h at 170° C. The reaction medium is diluted with water, extracted with TBME, the organic layer is washed with a saturated aqueous NaCl solution, dried over MgSO₄, filtered and evaporated. 20.7 g of desired product are isolated after chromatography on silica, eluting with an ethyl acetate/cyclohexane mixture (30:70 v/v).

### B/ (4-Hydroxy-3-trifluoromethyl-phenyl)-tertbutyl carbamate

32.4 g of desired product are isolated after following General Procedure E to hydrogenate 33.4 g of compound obtained such as described in the preceding step, followed by protection of the aniline thus obtained by a BOC group in accordance with General Procedure F.

### C/ [4-(4-Nitro-phenoxy)-3-trifluoromethyl-phenyl]-tertbutyl carbamate

Following General Procedure O, 20 g of compound of the preceding step are condensed on 11.3 g of 1-chloro-4-nitrobenzene. 3.2 g of desired product are isolated after chromatography on silica, eluting with a cyclohexane/ethyl acetate mixture (90:10 v/v).

### D/ 4-(4-Nitro-phenoxy)-3-trifluoromethyl-phenylamine

Following General Procedure C, 6.1 g of desired product are obtained in the form of a free base from 9.1 g of compound obtained such as described in the preceding step.

### E/ 1-(1-Ethyl-propyl)-3-[4-(4-nitro-phenoxy)-3-trifluoromethyl-phenyl]-urea

Following General Procedure H, 3.7 g of desired product are obtained from the compound of the preceding step.

### F/ 1-[4-(4-Amino-phenoxy)-3-trifluoromethyl-phenyl]-3-(1-ethyl-propyl)-urea

3 g of compound obtained in the preceding step are reduced in the presence of NH₄Cl (2 eq) and of powder Zn (20 eq) in MeOH (200 ml), for 15 h at AT, followed by filtering on celite and concentration in vacuo. 2.65 g of desired product are isolated after chromatography on silica, eluting with a cyclohexane/ethyl acetate mixture (50:50 v/v).

### Preparation 95

### 1-[4-(4-Amino-2-methoxymethyl-phenoxy)-phenyl]-3-dimethylamino-urea

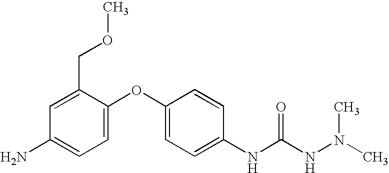

### A/ {4-[4-(3-dimethylamino-ureido)-phenoxy]-3-methoxymethyl-phenyl}-tertbutyl carbamate

5.7 g of compound obtained such as described under Preparation 73, step C, in solution in THF (60 ml), is added to a solution of CDI (6 eq) in THF (60 ml), then TEA (2 eq) is added and stirred 1 h at AT, and finally N,N-dimethylhydrazine (6 eq) is added and stirred a further 24 h at AT. The reaction medium is concentrated in vacuo, redissolved in DCM, the organic layer is washed with water, dried over MgSO₄, filtered and evaporated to dryness. 3.17 g of desired product are isolated after chromatography on silica, eluting with a DCM/ethyl acetate mixture (96:4 (v/v).

### B/ 1-[4-(4-Amino-2-methoxymethyl-phenoxy-phenyl]-3-dimethylamino-urea

Following General Procedure C, 3.7 g of desired product are obtained in the form of a TFA salt from the compound of the preceding step.

### Preparation 96

1-[4-(4-Amino-2,6-dimethyl-phenoxy)-phenyl]-3-isopropyl-urea

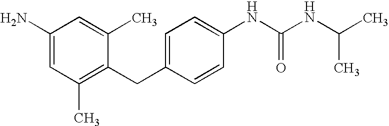

### A/ (4-Hydroxy-3,5-dimethyl-phenyl)-tertbutyl carbamate

A mixture of 2,6-dimethyl-4-nitrophenol (6 g), of 10% palladium on charcoal (1.8 g) and of (BOC)₂O (1.1 eq) in THF (260 ml) is stirred in a hydrogen atmosphere for 2 h at AT. The reaction medium is filtered, the filtrate concentrated in vacuo, redissolved in pentane, and the precipitate is filtered and dried. 5.64 g of desired product are obtained in the form of a white powder.

### B/ [3,5-Dimethyl-4-(4-nitro-phenoxy)-phenyl]-tertbutyl carbamate

Following General Procedure O, 3 g of desired product are obtained in the form of yellow crystals, by condensing the compound of the preceding step on 4-fluoronitrobenzene (1.93 ml).

### C/ [4-(4 Amino-phenoxy)-3,5-dimethyl-phenyl]-tertbutyl carbamate

Following General Procedure E, 3.6 g of desired product are obtained in the form of an orange solid from 4.3 g of compound obtained such as described in the preceding step.

### D/ {4-[4-(3-Isopropyl-ureido)-phenoxy]-3,5-dimethyl-phenyl}-tertbutyl carbamate

Following General Procedure N, 0.96 g of desired product are obtained from 0.8 g of compound obtained such as described in the preceding step.

### E/ 1-[4-(4-Amino-2,6-dimethyl-phenoxy)-phenyl]-3-isopropyl-urea

Following General Procedure C, 0.66 g of desired product are obtained from 0.94 g of compound produced such as described in the preceding step.

### Preparation 97

### 1-[4-(4-Amino-2,5-dimethyl-phenoxy)-phenyl]-3-isopropyl-urea

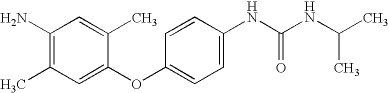

### A/ (4-Hydroxy-2,5-dimethyl-phenyl)-tertbutyl carbamate

Following General Procedure F, 5.9 g of desired product are obtained from 5 g of 4-amino-2,5-dimethylphenol.

### B/ [2,5-Dimethyl-4-(4-nitro-phenoxy)-phenyl]-tertbutyl carbamate

A solution of 5.2 g of compound of the preceding step in THF (50 ml) is heated under reflux for 1 h in the presence of NaOh pellets (1.1 eq), then 4-fluoronitrobenzene (1.2 eq) is added and heating under reflux continued for a further 4 h. The solvent is evaporated in vacuo, the residue redissolved in water, extracted with ethyl acetate and the organic layer is washed with an aqueous NaCl solution, dried over MgSO₄, filtered and evaporated. 3.4 g of desired product are isolated in the form of a pale yellow solid, after chromatography on silica, eluting with DCM.

### C/ [4-(4-Amino-phenoxy)-2,5-dimethyl-phenyl]-tertbutyl carbamate

Following General Procedure E, 0.75 g of desired product are obtained from 1.8 g of compound obtained in the preceding step.

### D/ {4-[4-(3-Isopropyl-ureido)-phenoxy]-2,5-dimethyl-phenyl}-tertbutyl carbamate

Following General Procedure N, 0.6 g of desired product are obtained in the form of a pinkish-beige solid, from the compound obtained in the preceding step.

### E/ 1-[4-(4-Amino-2,5-dimethyl-phenoxy)-phenyl]-3-isopropyl-urea

Following General Procedure C, 0.4 g of desired product are obtained from the compound produced in the preceding step.

### Preparation 98

### 1-[4-(4-Amino-2-trifluoromethyl-phenoxy)-phenyl]-3-(1-ethyl-propyl)-urea

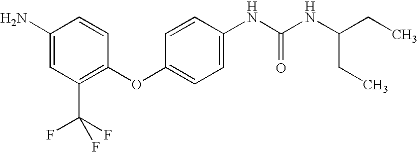

### A/ [4-(4-Amino-phenoxy)-3-trifluoromethyl-phenyl]-tertbutyl carbamate

8.4 g of compound obtained such as described under Preparation 94, step B, are reduced in the presence of NH₄Cl (2 eq) and of powder Zn (20 eq) in MeOH (200 ml) at AT for 15 h. The reaction medium is filtered on celite and concentrated in vacuo. 6.6 g of desired product are isolated after chromatography on silica, eluting with a cyclohexane/ethyl acetate mixture (7:3 v/v).

### B/ (4-{4-[3-(1-Ethyl-propyl)-ureido]-phenoxy}-3-trifluoromethyl-phenyl)-tertbutyl carbamate

Following General Procedure H, 9.6 g of desired product are obtained from the compound afforded by the preceding step.

### C/ 1-[4-(4-Amino-2-trifluoromethyl-phenoxy)-phenyl]-3-(1-ethyl-propyl)-urea

Following General Procedure C, 4.4 g of desired product are obtained from the compound afforded by the preceding step.

### Preparation 99

### N-[4-(4-Amino-phenoxy)-3-methoxy-phenyl]-2-dimethylamino-acetamide

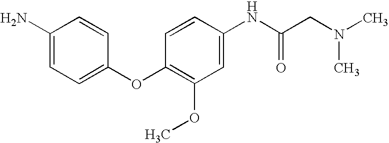

### A/ 2-Dimethylamino-N-[3-methoxy-4-(4-nitro-phenoxy)-phenyl]-acetamide

A mixture of 1.9 g of compound obtained such as described under Preparation 70, Method I, step C, of N,N-dimethylglycine (1.2 eq), of HOBT (1.3 eq), of EDCI (1.3 eq) and of DIEA (3.5 eq) in DCM (10 ml) is stirred at AT for 20 h. The organic layer is washed with water, with an aqueous 1N NaOH solution then with a saturated aqueous NaCl solution, dried over MgSO₄, filtered and evaporated. 2 g of desired product are obtained in the form of a yellow solid.

### B/ N-[4-(4-Amino-phenoxy)-3-methoxy-phenyl]-2-dimethylamino-acetamide

The compound of the preceding step is treated according to General Procedure E. 0.67 g of desired product are isolated in HCl salt form, after chromatography on silica eluting with a DCM/MeOH mixture (95:5 v/v), followed by treatment with a solution of HCl in diethyl ether.

### Preparation 100

### 1-[4-(4-Amino-phenoxy)-2-hydroxymethyl-phenyl]-3-isopropyl-urea

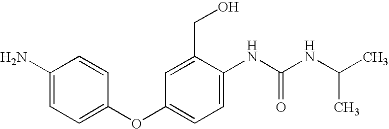

0.5 g of compound obtained such as described under Preparation 77 is heated under reflux in THF (15 ml), in the presence of LAH (4 eq). The reaction medium is cooled, an aqueous Na₂SO₄ solution is added, filtered and the filtrate evaporated. 430 mg of desired product are obtained in the form of a beige solid.

### Preparation 101

### 1-[4-(4-Amino-phenoxy)-2-methoxy-phenyl]-3-isopropyl-urea

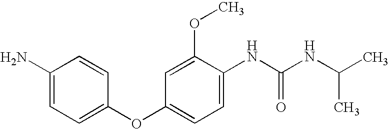

### A/ [4-(4-Amino-3-methoxy-phenoxy)-phenyl]-tertbutyl carbamate

Following General Procedure E, 2.0 g of desired product are obtained from 3 g of compound obtained such as described under Preparation 89, step A.

### B/ (4-{4-[3-(1-Ethyl-propyl)-ureido]-3-methoxy-phenoxy}-phenyl)-tertbutyl carbamate

Following General Procedure N, 2.0 g of desired product are obtained from the compound of the preceding step.

### C/ 1-[4-(4-Amino-phenoxy)-2-methoxy-phenyl]-3-isopropyl-urea

Following General Procedure C, 2.4 g of desired product are obtained in the form of a TFA salt, from the compound of the preceding step.

### Preparation 102

### 1-[4-(4-Amino-phenoxy)-3-methoxy-phenyl]-3-isopropyl-urea

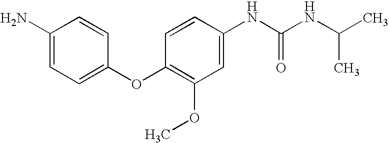

### A/ 1-Isopropyl-3-[2-methoxy-4-(4-nitro-phenoxy)-phenyl]-urea

Following General Procedure N, 1.9 g of desired product are obtained from 2 g of compound produced such as described under Preparation 70, Method I, step C.

### B/ 1-[4-(4-Amino-phenoxy)-3-methoxy-phenyl]-3-isopropyl-urea

Following General Procedure E, 1.6 g of desired product are obtained from the compound of the preceding step.

### Preparation 103

### (1-Ethyl-propyl)-carbamate of 4-(4-amino-2-methyl-phenoxy)-phenyl

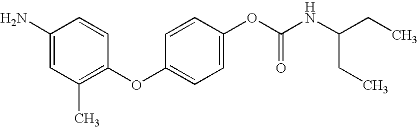

### A/ 1-(4-Methoxy-phenoxy)-2-methyl-4-nitro-benzene

Following General Procedure O, 16.7 g of desired product are obtained in the form of a yellow oil, by condensing 16 g of 4-methoxyphenol on 10 g of 2-fluoro-5-nitrotoluene.

### B/ 4-(2-Methyl-4-nitro-phenoxy)-phenol

To a suspension of AlCl₃ (6.6 eq) in ethanethiol (114 ml) cooled to around −5° C., the compound obtained in the preceding step in 46 ml of ethanethiol is added dropwise, and stirred 3 h at 0° C. The reaction medium is poured slowly, at 0° C., onto an aqueous 3N HCl solution, extracted with DCM, and the organic layer is dried over MgSO₄, filtered and evaporated in vacuo. The residue is redissolved in pentane, and the precipitate obtained is collected and dried. 7.9 g of desired product are isolated in the form of a yellow powder.

### C/ Chloromethyl carbonate and 4-(2-methyl-4-nitro-phenoxy)-phenyl

Following Patonay, Synth. Commun., 20(18), 1990, pp 2865-2885, to a solution cooled to around −10° C. of chloromethyl chloroformate (1.05 eq) in DCM (24 ml), is added a mixture of 4 g of compound obtained in the preceding step and of TEA (1.05 eq) in 8 ml DCM, and stirred 2 h at a temperature of below 5° C. The precipitate formed is filtered, the filtrate washed with an aqueous NaHCO₃ solution then with water, the organic layer is then separated, dried over MgSO₄, filtered and evaporated in vacuo. 4.9 g of desired product are obtained in the form of a white powder.

### D/ (1-Ethyl-propyl)-carbamate of 4-(2-methyl-4-nitro-phenoxy)-phenyl

The compound of the preceding step is reacted with 2.6 eq of 1-ethylpropylamine in 20 ml THF for 48 h at AT and 5 h under reflux. The solvent is evaporated in vacuo, the residue redissolved in DCM, washed with a saturated aquoues NaHCO₃ solution, with water and finally with a 1N aqueous HCl solution, the organic layer is dried over MgSO₄, filtered and evaporated. 3.47 g of desired product are isolated in the form of an off-white powder, after chromatography on silica eluting with a DCM/acetone mixture (99:1 v/v), followed by precipitation in pentane.

### E/ (1-Ethyl-propyl)-carbamate of 4-(4-amino-2-methyl-phenoxy)-phenyl

Following General Procedure E, from the compound of the preceding step 3.2 g of desired product are obtained as HCl salt, in the form of a white powder, by precipitating the free base with a HCl/diethyl ether mixture.

### Preparation 104

### 2-(4-Amino-phenoxy)-5-[3-(1-ethyl-propyl)-ureido]-N-methyl-benzamide

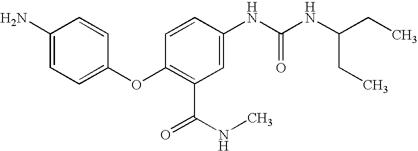

### A/ 2-(4-tert-Butoxycarbonylamino-phenoxy)-5-[3-(1-ethyl-propyl-ureido]-benzoic acid

Following General Procedure A, 4.1 g of desired product are obtained from 4.3 g of compound obtained such as described under Preparation 77, step D.

### B/ (4-{4-[3-(1-Ethyl-propyl)-ureido]-2-methylcarbamoyl-phenoxy}-phenyl)-tertbutyl carbamate

A mixture of compound obtained in the preceding step, of HOBT (1.2 eq), of DIEA (3 eq), of methylamine (1.5 eq) and of EDCI (1.1 eq) in 40 ml DMF is stirred for 18 h at AT. The reaction medium is concentrated, the residue redissolved in an aqueous 1 N HCl solution, the precipitate formed is filtered and dissolved in ethyl acetate. The organic layer is washed with an aqueous ammonia solution, dried over MgSO₄, filtered and evaporated in vacuo. 3.9 g of desired product are obtained in the form of an off-white solid.

### C/ 2-(4-Amino-phenoxy)-5-[3-(1-ethyl-propyl)-ureido]-N-methyl-benzamide

Following General Procedure C, 2.9 g of desired product are obtained in free base form, from the compound of the preceding step.

### Preparation 105

### 1-[4-(4-Amino-phenylsulfanyl)-phenyl]-3-isopropyl-urea

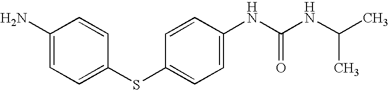

### A/ 1-Isopropyl-3-[4-(4-nitro-phenylsulfanyl)-phenyl]-urea

3.02 g of 4-amino-4′-nitrophenyldisulfide are reacted with 1.2 ml of isopropyl isocyanate in 24 ml of anhydrous pyridine, the reaction medium is redissolved in DCM, the insoluble is filtered, washed with an aqueous HCl solution then with water. The DCM phase is washed with an aqueous HCl solution, added back to the insoluble and the whole is evaporated in vacuo. 2.67 g of desired product are isolated, which is used as such.

### B/ 1-[4-(4-Amino-phenylsulfanyl-phenyl]-3-isopropyl-urea

The desired product is obtained by treating the compound of the preceding step in accordance with General Procedure E.

### Preparation 106

### 1-[4-(4-Amino-benzyl)-phenyl]-3-isopropyl-urea

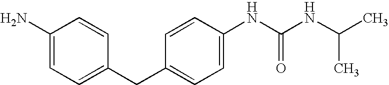

### A/ [4-(4-Amino-benzyl)-phenyl]-tertbutyl carbamate

Following General Procedure F, 3.7 g of desired product are obtained from 5 g of 4,4′-methylenedianiline.

### B/ (4-{4-[3-(1-Ethyl-propyl)-ureido]-benzyl}-phenyl)-tertbutyl carbamate

Following General Procedure N, 1.7 g of desired product are obtained from 2 g of compound of the preceding step.

### C/ 1-[4-(4-Amino-benzyl)-phenyl]-3-isopropyl-urea

Following General Procedure C, 1.1 g of desired product are obtained from the compound of the preceding step.

### Preparation 107

### 1-[4-(4-Amino-3-fluoro-phenoxy)-3-ethoxy-phenyl]-3-(1-ethyl-propyl)-urea

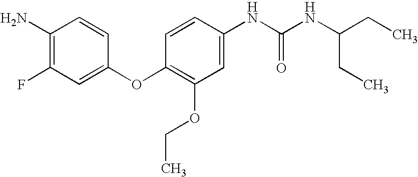

### A/ 2-Fluoro-4-(2-methoxy-4-nitro-phenoxy)-phenylamine

A mixture of 10 g of compound obtained such as described under Preparation 85, step A, 8.25 g of 2-chloro-5-nitroanisole and 2.47 g of flaked KOH in 80 ml of anhydrous DMF is heated under reflux for 48 h. After in vacuo concentration, the residue is redissolved in a water/TBME mixture, the precipitate formed and the organic layer are collected, the organic layer is washed with water, dried over MgSO₄ and evaporated. 3.97 g of desired product are isolated after chromatography on silica eluting with a DCM/cyclohexane mixture (10:10 v/v), then with DCM alone.

### B/ 2-(4-Amino-3-fluoro-phenoxy)-5-nitro-phenol

A mixture of 3 g of compound obtained in the preceding step and of HBr (70 ml at 47%) is heated for 3 h at 150° C. The reaction medium is poured onto a water/ice mixture, extracted a first time with ethyl acetate, the aqueous phase is basified with an aqueous ammonia solution, and extracted a further time with ethyl acetate. The organic phases are grouped together and washed with an aqueous ammonia solution, dried over MgSO₄, filtered and evaporated in vacuo. 3 g of desired product are obtained, which is used as such.

### C/ [2-Fluoro-4-(2-hydroxy-4-nitro-phenoxy)-phenyl]-tertbutyl carbamate

General Procedure F is used to treat the compound of the preceding step. 1.5 g of desired product are isolated after chromatography on silica eluting with a cyclohexane/ethyl acetate mixture (85:15 v/v).

### D/ [4-(2-Ethoxy-4-nitro-phenoxy)-2-fluoro-phenyl]-tertbutyl carbamate

A suspension of 1.43 g of compound obtained in the preceding step and of K₂CO₃ (1.5 eq) in DMF (15 ml) is stirred 15 min at AT, iodoethane (1.1 eq) is added and stirred for 2 h at AT. The solvent is evaporated, the residue redissolved in TBME, washed with water, the organic layer is dried over MgSO₄, filtered and evaporated in vacuo. 1.55 g of desired product are isolated after chromatography on silica, eluting with a cyclohexane/ethyl acetate mixture (90:10 v/v).

### E/ [4-(4-Amino-2-ethoxy-phenoxy)-2-fluoro-phenyl]-tertbutyl carbamate

Following General Procedure E, 1 g of desired product are obtained from 1.4 g of compound produced in the preceding step.

### F/ (4-{2-Ethoxy-4-[3-(1-ethyl-propyl)-ureido]-phenoxy}-2-fluoro-phenyl)-tertbutyl carbamate

General Procedure H is used to treat the compound of the preceding step. 1.1 g of desired product are isolated after chromatography on silica eluting with a cyclohexane/ethyl acetate mixture (80:20 v/v).

### G/ 1-[4-(4-Amino-3-fluoro-phenoxy)-3-ethoxy-phenyl]-3-(1-ethyl-propyl)-urea

Following General Procedure C, 0.86 g of desired product are obtained in the form of a free base, from the compound of the preceding step.

### Preparation 108

### 1-[4-(4-Amino-3-fluoro-phenoxy)-3-ethoxy-phenyl]-3-(1-ethyl-propyl)-urea

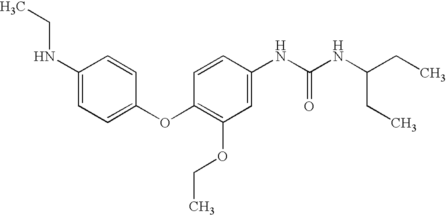

### A/ N-(4-{2-Ethoxy-4-[3-(1-ethyl-propyl)-ureido]-phenoxy}-phenyl)-acetamide

1.3 g of compound obtained such as described under Preparation 71, in 10 ml of acetic acid, is stirred 1 h at AT in the presence of 3 ml of acetic anhydride. The reaction medium is diluted in water, and the precipitate formed is filtered and dried. 1.36 g of desired product are isolated, which is used as such.

### B/ 1-[4-(4-Amino-3-fluoro-phenoxy)-3-ethoxy-phenyl]-3-(1-ethyl-propyl)-urea

The compound obtained in the preceding step is reacted with LAH (6 eq) in THF (40 ml), for 24 h at 60° C. The reaction medium is diluted with water, concentrated in vacuo, the residue is redissolved in DCM and the organic layer is washed with water, dried over MgSO₄, filtered and evaporated. 1.31 g of desired product are isolated after chromatography on silica eluting with a cyclohexane/ethyl acetate mixture (60:40).

### Preparation 109

### 1-(1-Ethyl-propyl)-3-{4-[4-(2-hydroxy-ethylamino)-phenoxy]-3-methoxy-phenyl}-urea

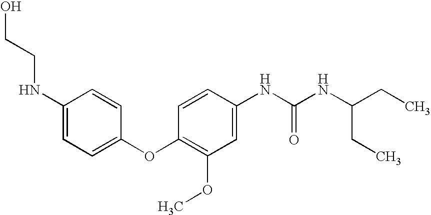

A solution of 0.5 g of compound obtained such as described under Preparation 70, in 10 ml DMF, is heated at 80° C. for 48 h in the presence of 2-bromoethanol (2.4 eq) and DIEA (7.2 eq). The reaction medium is concentrated in vacuo and 230 mg of desired product are isolated after chromatography on silica eluting with a DCM/MeOH mixture (95:5 v/v).

### Preparation 110

### 1-(1-Ethyl-propyl)-3-{3-methoxy-4-[4-(3,3,3-trifluoro-propylamino)-phenoxy]-phenyl}-urea

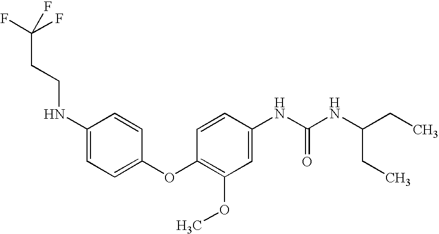

In a sealed tube, a solution of 0.75 g of compound obtained such as described under Preparation 70, in 5 ml DMF, is heated at 70° C. for 24 h in the presence of trifluoroiodopropane (1.6 eq) and DIEA (3 eq). The reaction medium is then diluted with TBME, washed with an aqueous NaHCO₃ solution and with water, and the organic layer is dried over MgSO₄, filtered and concentrated in vacuo. 0.74 g of desired product are obtained, which is used as such.

### Preparation 111

### 1-(1-Ethyl-propyl)-3-{3-methoxy-4-[4-(3-methoxy-propylamino)-phenoxy]-phenyl}-urea

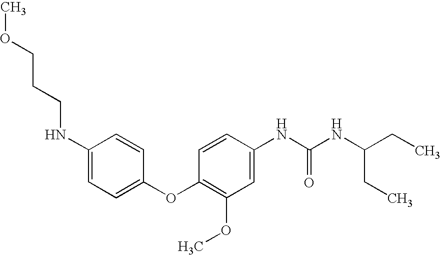

### A/ 3-methoxy-propan-1-ol

100 g of propanediol are reacted with 9.3 g of sodium for 1 h at AT, then 25.6 ml of methyl iodide are added dropwise and stirred 24 h at AT. 24.1 g of desired product are obtained after distilling under AP at 134° C.

### B/ 1-Bromo-3-methoxy-propane

On the compound of the preceding step is added dropwise 11.2 ml of PBr₃ keeping the temperature to below 60° C., the mixture is stirred 30 min at 60° C., then poured into water, extracted with DCM, and the organic layer is dried over MgSO₄, filtered and evaporated. 10.6 g of desired product are obtained after distilling under AP at 108-115° C.

### C/ 1-(1-Ethyl-propyl)-3-{3-methoxy-4-[4-(3-methoxy-propylamino)-phenoxy]-phenyl}-urea

A solution of 0.85 g of compound obtained such as described under Preparation 70, in 2 ml DMF, is heated at 80° C. for 18 h in the presence of 3-methoxy-1-bromopropane (1.2 eq) and DIEA (1.2 eq). The reaction medium is concentrated in vacuo, and 410 mg of desired product are isolated after chromatography on silica eluting with a cyclohexane/ethyl acetate mixture (70:30 v/v).

### Preparation 112

### 1-[4-(4-Amino-phenoxy)-3-isopropyl-phenyl]-3-(1-ethyl-propyl)-urea

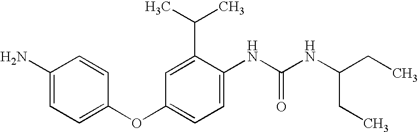

### A/ 2-Isopropyl-4-nitro-phenol

To a solution of 2-isopropyl-phenol (43.6 g) in 80 ml ACN cooled to around −10° C., is added 25.6 g of ammonium nitrite, then dropwise 49 ml of trifluoroacetic anhydride keeping the temperature to between −5° C. and −10° C., followed by stirring 1 h at this temperature. An ice/water mixture is added to the reaction medium which is washed with pentane, extracted with DCM, then the organic layer is extracted with an aqueous NaOH solution and the aqueous layer is acidified. The product of this aqueous layer is extracted with DCM, and the organic layer is evaporated in vacuo. 7.9 g of desired product are isolated after chromatography on silica eluting with DCM.

### B/ 4-Amino-2-isopropyl-phenol

Following General Procedure E, 7.4 g of desired product are obtained from 12 g of compound obtained such as described in the preceding step.

### C/ 1-(1-Ethyl-propyl)-3-(4-hydroxy-3-isopropyl-phenyl)-urea

Following General Procedure H, 6.4 g of compound obtained in the preceding step are caused to react. The reaction medium is concentrated, the residue redissolved in an aqueous NaOH solution, washed with TBME, the aqueous layer is acidified with concentrated HCl, the product of the aqueous layer extracted with TBME and the organic layer is evaporated to dryness. 5.6 g of desired product are obtained, which is used as such.

### D/ 1-(1-Ethyl-propyl)-3-[3-isopropyl-4-(4-nitro-phenoxy-phenyl]-urea

Following General Procedure O, the compound obtained in the preceding step is condensed at AT on 4-fluoronitrobenzene (24 mmol). The reaction medium is concentrated, the residue redissolved in an aqueous NaOH solution, extracted with TBME and the organic layer is evaporated to dryness. 4.7 g of desired product are obtained after crystallization in diisopropyl ether.

### E/ 1-[4-(4-Amino-phenoxy)-3-isopropyl-phenyl]-3-(1-ethyl-propyl-urea

Following General Procedure E, 4.3 g of desired product are obtained from the compound of the preceding step.

### Preparation 113

### 1-[3-Chloro-4-(4-ethylamino-phenoxy)-phenyl]-3-(1-ethyl-propyl)-urea

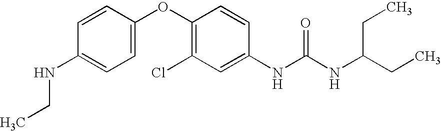

### A/ N-(4-{2-Chloro-4-[3-(1-ethyl-propyl)-ureido]-phenoxy}-phenyl)-acetamide

0.7 of compound obtained such as described under Preparation 80, in 0.35 ml of acetic acid, is stirred for 24 h at AT, in the presence of 0.35 ml of acetic anhydride. The reaction medium is diluted in water, and the precipitate formed is filtered and dried. 0.35 g of desired product are isolated in the form of a white powder which is used as such.

### B/ 1-[3-Chloro-4-(4-ethylamino-phenoxy)-phenyl]-3-(1-ethyl-propyl)-urea

The compound obtained in the preceding step is reacted with LAH (3 eq) in THF (25 ml), for 7 h under reflux. After adding a few drops of a saturated aqueous Na₂SO₄ solution to the reaction medium, it is evaporated in vacuo, the residue redissolved in an aqueous ammonia solution, the product extracted with DCM and the organic layer is dried over MgSO₄, filtered and evaporated. 0.22 g of desired product are isolated after chromatography on silica eluting with a DCM/MeOH/NH₄OH mixture (95:5:0.05 v/v/v).

### Preparation 114

### 1-(1-Ethyl-propyl)-3-{3-methoxy-4-[4-(4,4,4-trifluoro-butylamino)-phenoxy]-phenyl}-urea

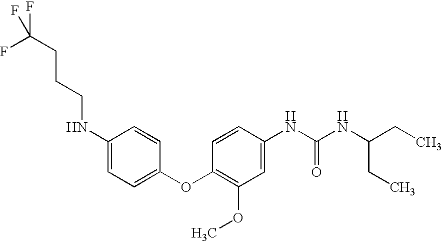

A solution of 0.5 g of compound obtained such as described under Preparation 70, in 2 ml DMF, is heated at 80° C. for 18 h in the presence of 4,4,4-trifluoro-1-bromobutane (1.2 eq) and DIEA (1.2 eq). The reaction medium is concentrated in vacuo and 394 g of desired product are isolated after chromatography on silica eluting with a cyclohexane/ethyl acetate mixture (70:30 v/v).

### Preparation 115

### 1-[3-(4-Amino-phenoxy)-4-methoxy-phenyl]-3-(1-ethyl-propyl)-urea

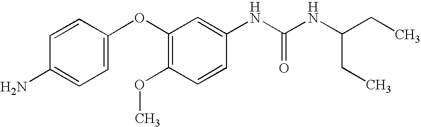

### A/ (3-Hydroxy-4-methoxy-phenyl)-tertbutyl carbamate

13.4 g of desired product are isolated after hydrogenating 20 g of 2-methoxy-5-nitrophenol in accordance with General Procedure E, followed by protection of the aniline thus obtained by a BOC group following General Procedure F.

### B/ [4-Methoxy-3-(4-nitro-phenoxy)-phenyl]-tertbutyl carbamate

Following General Procedure O, the compound obtained in the preceding step is condensed on 11.49 g of 4-chloronitrobenzene. 12.2 g of desired product are isolated after chromatography on silica eluting with DCM.

### C/ 4-Methoxy-3-(4-nitro-phenoxy)-phenylamine

Following General Procedure C, 10.2 g of desired product are obtained in TFA salt form, from the compound of the preceding step.

### D/ 1-(1-Ethyl-propyl)-3-[4-methoxy-3-(4-nitro-phenoxy)-phenyl]-urea

Following General Procedure H and from 5.1 g of compound of the preceding step, 5 g of desired product are isolated after chromatography on silica eluting with a cyclohexane/ethyl acetate mixture (60:40 v/v).

### E/ 1-[3-(4-Amino-phenoxy)-4-methoxy-phenyl]-3-(1-ethyl-propyl)-urea

General Procedure E is used to treat the compound of the preceding step. 4.3 g of desired product are isolated in HCl salt form, after treatment with a HCl solution in diethyl ether.

### Preparation 116

### 1-[3-(4-Amino-2-methyl-phenoxy)-phenyl]-3-(1-ethyl-propyl)-urea

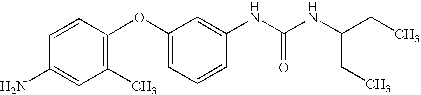

### A/ 3-N—BOC-aminophenol

Following General Procedure F, 45.4 of desired product are obtained in the form of a white powder, from 25 g of 3-aminophenol.

### B/ [3-(2-Methyl-4-nitro-phenoxy)-phenyl]-tertbutyl carbamate

Following General Procedure O, 15 g of 3-N—BOC-aminophenol are condensed on 16 g of 2-chloro-5-nitrotoluene. 13.1 g of desired product are isolated after chromatography on silica eluting with a cyclohexane/ethyl acetate mixture (90:10 v/v).

### C/ 3-(2-Methyl-4-nitro-phenoxy)-phenylamine

Following General Procedure C, 8.74 g of desired product are obtained in the form of a free base, from the compound of the preceding step.

### D/ 1-(1-Ethyl-propyl)-3-[3-(2-methyl-4-nitro-phenoxy)-phenyl]-urea

General Procedure H is used to treat 4 g of compound of the preceding step. 3.68 g of desired product are isolated after chromatography on silica eluting with a cyclohexane/ethyl acetate mixture (77:23 v/v).

### E/ 1-[3-(4-Amino-2-methyl-phenoxy)-phenyl]-3-(1-ethyl-propyl)-urea

General Procedure E is followed to treat the compound of the preceding step. 3.1 g of desired product are isolated in HCl salt form, after treatment with a HCl solution in diethyl ether.

### Preparation 117

### 1-[4-(3-Amino-phenoxy)-3-methoxy-phenyl]-3-dimethylamino-urea

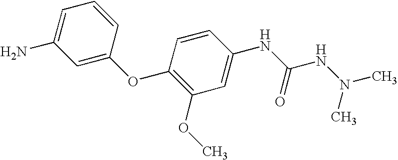

### A/ [3-(2-Methoxy-4-nitro-phenoxy)-phenyl]-tertbutyl carbamate

Following General Procedure O, 3-BOC-amino-phenol is condensed on 8.96 g of 2-chloro-5-nitroanisole. 8.7 g of desired product are obtained after chromatography on silica eluting with a DCM/cyclohexane mixture (3:1 v/v) then with DCM.

### B/ [3-(4-Amino-2-methoxy-phenoxy)-phenyl]-tertbutyl carbamate

Following General Procedure E, 6.93 g of desired product are obtained from the compound of the preceding step.

### C/ {3-[4-(3-dimethylamino-ureido)-2-methoxy-phenoxy]-phenyl}-tertbutyl carbamate

To a solution of CDI (6 eq) in THF (65 ml) cooled to around −10° C., are added the compound of the preceding step in THF (65 ml) dropwise, then N,N-dimethylhydrazine (6 eq) in small portions, followed by stirring 1 h at 0° C. and 18 h at AT. After concentration in vacuo, the residue is redissolved in DCM, the organic layer washed with water, dried over MgSO₄, filtered and evaporated. 2.35 g of desired product are isolated after chromatography on silica eluting with a cyclohexane/ethyl acetate mixture (40:60 v/v).

### D/ 1-[4-(3-Amino-phenoxy)-3-methoxy-phenyl]-3-dimethylamino-urea

The desired product is isolated in TFA salt form by following General Procedure C to treat the compound of the preceding step.

### Preparation 118

### N-(4-{4-[3-(1-Ethyl-propyl)-ureido]-phenoxy}-3-methyl-phenyl)-4-Piperidin-4-yloxy)-benzamide

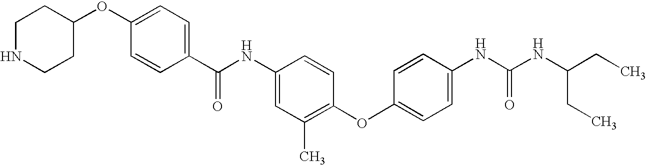

### A/ 4-(1-Benzyl-Piperidin-4-yloxy)-N-(4-{4-[3-(1-ethyl-propyl)-ureido]-phenoxy}-3-methyl-phenyl)-benzamide

Following General Procedure I, 3 g of 4-(1-Benzyl-Piperidin-4-yloxy)-benzoic acid (Preparation 11) are reacted with 3.5 g of 1-[4-(4-Amino-2-methyl-phenoxy)-phenyl]-3-(1-ethyl-propyl)-urea (Preparation 75), the solvent is evaporated in vacuo at 60° C., the reaction medium is held in a vacuum at 60° C. for 3 h then 24 h at AT. 5.7 g of desired product are obtained after chromatography on silica eluting with a DCM/MeOH/NH₄OH mixture (95:4:1 v/v/v).

### B/ N-(4-{4-[3-(1-Ethyl-propyl)-ureido]-phenoxy}-3-methyl-phenyl)-4-(Piperidin-4-yloxy)-benzamide

4.5 g of desired product are obtained by following General Procedure D to treat the compound of the preceding step.

### Preparation 119

### N-(4-{4-[3-(1-Ethyl-propyl)-ureido]-2-methoxy-phenoxy}-phenyl)-4-(Piperidin-4-yloxy)-benzamide

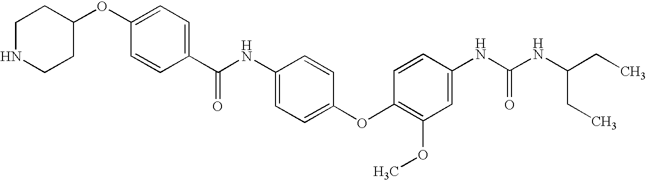

### A/ 4-(1-Benzyl-Piperidin-4-yloxy)-N-(4-{4-[3-(1-ethyl-propyl)-ureido]-2-methoxy-phenoxy}-phenyl)-benzamide

Following General Procedure L4, 2.50 g of 4-(1-Benzyl-Piperidin-4-yloxy)-benzoic acid (Preparation 11) are reacted with 2.84 g of 1-[4-(4-Amino-phenoxy)-3-methoxy-phenyl]-3-(1-ethyl-propyl)-urea (Preparation 70), in the presence of a mixture of EDCI/HOBT. After evaporation in vacuo, the desired product is isolated in the form of a free base after chromatography on silica eluting with a DCM/MeOH/NH₄OH mixture (90:10:0.1 v/v/v).

### B/ N-(4-{4-[3-(1-Ethyl-propyl)-ureido]-2-methoxy-phenoxy}-phenyl)-4-(Piperidin-4-yloxy)-benzamide

4.3 g of desired product are obtained by following General Procedure D to treat the compound of the preceding step.

### Preparation 120

### 4-(8-Aza-bicyclo[3.2.1]oct-(3-endo)-yloxy)-N-(4-{4-[3-(1-ethyl-propyl)-ureido]-2-methoxy-phenoxy}-phenyl)-benzamide

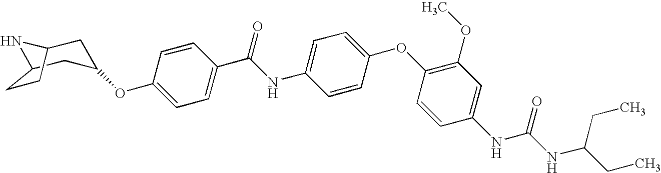

### A/ 4-(8-Aza-bicyclo[3.2.1]oct-(3-endo)-yloxy)-benzonitrile

A mixture of 37 g of compound obtained such as described under Preparation 27, Method I, step A, 96.1 g of K₂CO₃ and 100 ml of chloroethyl chloroformate in 450 ml of 1,2-dichloroethane is heated under reflux for 8 h. The insoluble is filtered, the filtrate evaporated in vacuo, 370 ml of MeOH are added followed by stirring for 15 h at AT. After evaporation in vacuo, the residue is redissolved in water, washed with TBME, the aqueous layer is basified, extracted with TBME and the last organic layer is dried over MgSO₄, filtered and evaporated. 30.7 g of desired product are isolated.

### B/ 4-(8-Aza-bicyclo[3.2.1]oct-(3-endo)-yloxy)-benzoic acid

7 g of desired product are isolated in HCl salt form, using General Procedure B to treat 6.4 g of compound of the preceding step.

### C/ (3-endo)-(4-Carboxy-phenoxy)-8-aza-bicyclo[3.2.1]octane-8-tertbutyl carboxylate

To a mixture of 9.5 g of compound obtained such as described in the preceding step and 2.8 g of NaOH in water (105 ml)/tertbutanol (78 ml), is slowly added 10.6 g of (BOC)₂O followed by stirring at AT for 15 h. 9.5 g of KHSO₄ and 60 ml of water are added slowly, extracted with DCM and the organic layer is dried over MgSO₄, filtered and evaporated. 11 g of desired product are isolated.

### D/ (3-endo)-[4-(4-{4-[3-(1-Ethyl-propyl)-ureido]-2-methoxy-phenoxy}-phenylcarbamoyl)-phenoxy]-8-aza-bicyclo[3.2.1]octane-8-tertbutyl carboxylate

5.21 g of compound of the preceding step in 200 ml of DCM are stirred at AT for 1.5 h, in the presence of TBTU (1.3 eq), HOBT (1.3 eq) and DIEA (3 eq), washed with a dilute aqueous NaOH solution, with a dilute aqueous HCl solution, and the organic layer is dried over MgSO₄, filtered and evaporated. 5.15 g of 1-[4-(4-Amino-phenoxy)-3-methoxy-phenyl]-3-(1-ethyl-propyl)-urea (Preparation 70) in DMF are added, concentrated in vacuo at 60° C., the mixture kept at 60° C. in a vacuum for 8 h and at AT for 72 h. 7.6 g of desired product are obtained after chromatography on silica eluting with a DCM/MeOH/NH₄OH mixture (95:5:0.5 v/v/v).

### E/ 4-(8-Aza-bicyclo[3.2.1]oct-(3-endo)-yloxy)-N-(4-{4-[3-(1-ethyl-propyl)-ureido]-2-methoxy-phenoxy}-phenyl)-benzamide

8.1 g of desired product are obtained in TFA salt form by following General Procedure C to treat the compound of the preceding step.

### Preparation 121

### N-(4-{4-[3-(1-Ethyl-propyl)-ureido]-2-methoxy-phenoxy}-3-methyl-phenyl)-4-(Piperidin-4-yloxy)-benzamide

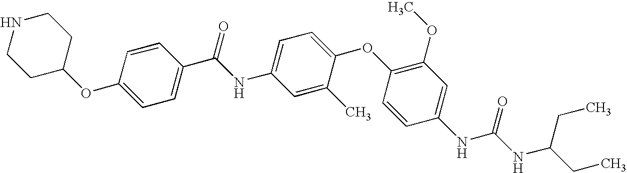

### A/ 4-(1-Benzyl-Piperidin-4-yloxy)-N-(4-{4-[3-(1-ethyl-propyl)-ureido]-2-methoxy-phenoxy}-3-methyl-phenyl)-benzamide

Following General Procedure I, 0.46 g of 4-(1-Benzyl-Piperidin-4-yloxy)-benzoic acid (Preparation 11) are reacted with 0.4 g of 1-[4-(4-Amino-2-methyl-phenoxy)-3-methoxy-phenyl]-3-(1-ethyl-propyl)-urea (Preparation 76). The reaction medium is stirred 30 min at AT, evaporated in vacuo at 60° C. and kept in a vacuum at 60° C. for 3 h. 0.6 g of desired product are obtained after chromatography on silica eluting with a DCM/MeOH/NH₄OH mixture (95:4:1 v/v/v).

### B/ N-(4-{4-[3-(1-Ethyl-propyl)-ureido]-2-methoxy-phenoxy}-3-methyl-phenyl)-4-(Piperidin-4-yloxy)-benzamide

0.44 g of desired product are obtained by following General Procedure D to treat the compound of the preceding step.

### Preparation 122

### N-(4-{4-[3-(1-Ethyl-propyl)-ureido]-2-methoxy-phenoxy}-phenyl)-N-(2-methoxy-ethyl)-4-(Piperidin-4-yloxy)-benzamide

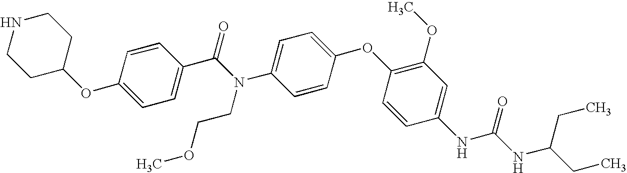

### A/ 4-(2-Methoxy-4-nitro-phenoxy)-phenylamine

The desired product is isolated in TFA salt form following General Procedure C to treat 6 g of compound obtained such as described under Preparation 70, Method II, step A.

### B/ 4-(Piperidin-4-yloxy)-benzoic acid

At AT under AP, 18 g of 4-(1-Benzyl-Piperidin-4-yloxy)-benzoic acid (Preparation 11) in 250 ml of water are treated with hydrogen, in the presence of 4.2 g of NaOH and 4 g of palladium hydroxide on charcoal. On completion of the reaction, the catalyst is filtered and the product obtained in solution is used as such.

### C/ 4-(4-Carboxy-phenoxy)-Piperidin-1-tertbutyl carboxylate

To the solution of the preceding step, 120 ml of tertbutanol, cooled to −10° C. are added, followed by the slow addition of 17 g of (BOC)₂O, stirring 15 h at AT, acidification to pH 4 with SO₂, extraction with TBME, and drying of the organic layer over MgSO₄, filtering and evaporation. 15.7 g of desired product are isolated.

### D/ 4-{4-[4-(2-Methoxy-4-nitro-phenoxy)-phenylcarbamoyl]-phenoxy}-Piperidine-1-tertbutyl carboxylate

Following General Procedure L1, 6.28 g of compound of the preceding step are reacted with 5.24 g of compound obtained at step A. 6.7 g of desired product are obtained after chromatography on silica eluting with a DCM/NH₄OH mixture (100:0.5 (v/v).

### E/ 4-(4-{(2-Methoxy-ethyl)-[4-(2-methoxy-4-nitro-phenoxy-phenyl]-carbamoyl}-phenoxy)-Piperidine-1-tertbutyl carboxylate

1.8 g of compound of the preceding step in 15 ml of dry DMSO is heated at 100° C. for 48 h in the presence of 5.2 g of Cs₂CO₃ and 0.9 ml of 2-bromo-ethyl-methylether. After diluting with water, extracting with ethyl acetate, the organic layer is dried over MgSO₄, filtered and evaporated. 0.9 g of desired product are obtained after chromatography on silica eluting with a cyclohexane/ethyl acetate mixture (50:50 v/v).

### F/ 4-{4-[[4-(4-Amino-2-methoxy-phenoxy)-phenyl]-(2-methoxy-ethyl)-carbamoyl]-phenoxy}-Piperidine-1-tertbutyl carboxylate

Following General Procedure E, 0.89 g of desired product are obtained from the compound of the preceding step.

### G/ 4-{4-[(4-{4-[3-(1-Ethyl-propyl)-ureido]-2-methoxy-phenoxy}-phenyl)-(2-methoxy-ethyl)carbamoyl]-phenoxy}-Piperidine-1-tertbutyl carboxylate

The compound of the preceding step is treated according to General Procedure H. 0.65 g of desired product are obtained after chromatography on silica eluting with a cyclohexane/ethyl acetate mixture (60:40 v/v).

### H/ N-(4-{4-[3-(1-Ethyl-propyl)-ureido]-2-methoxy-phenoxy}-phenyl)-N-(2-methoxy-ethyl)-4-(Piperidin-4-yloxy)-benzamide

The compound of the preceding step is treated according to General Procedure C. 0.51 g of desired product are obtained after chromatography on silica eluting with a DCM/MeOH/NH₄OH mixture (90:10:2 v/v/v).

### Preparation 123

### N-(4-{4-[3-(1-Ethyl-propyl)-ureido]-2-methoxy-phenoxy}-phenyl)-N-isobutyl-4-(Piperidin-4-yloxy)-benzamide

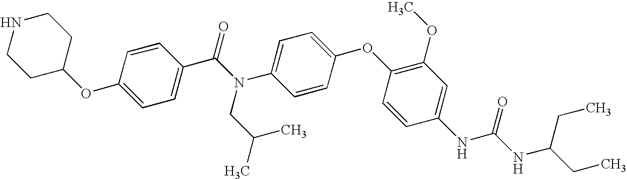

### A/ 4-(4-{Isobutyl-[4-(2-methoxy-4-nitro-phenoxy)-phenyl]-carbamoyl}-phenoxy)-Piperidine-1-tertbutyl carboxylate

1.8 g of compound obtained such as described under Preparation 122, step D, in 15 ml of dry DMSO, is heated at 80° C. for 10 h in the presence of 5.2 g of Cs₂CO₃ and 1.04 ml of isobutyl bromide. After diluting with water and extracting with ethyl acetate, the organic layer is dried over MgSO₄, filtered and evaporated. 1.14 g of desired product are obtained after chromatography on silica eluting with a cyclohexane/ethyl acetate mixture (70:30 v/v).

### B/ 4-(4-{[4-(4-Amino-2-methoxy-phenoxy)-phenyl]-isobutyl-carbamoyl}-phenoxy)-Piperidine-1-tertbutyl carboxylate

Following General Procedure E, 1.04 g of desired product are obtained from the compound of the preceding step.

### C/ 4-{4-[(4-{4-[3-(1-Ethyl-propyl-ureido]-2-methoxy-phenoxy}-phenyl)-isobutyl-carbamoyl]-phenoxy}-Piperidine-1-tertbutyl carboxylate

The compound of the preceding step is treated according to General Procedure H. 0.59 g of desired product are obtained after chromatography on silica eluting with a cyclohexane/ethyl acetate mixture (60:40 v/v), then with DCM/acetone (90:10 v/v).

### D/ N-(4-{4-[3-(1-Ethyl-propyl)-ureido]-2-methoxy-phenoxy}-phenyl)-N-isobutyl-4-(Piperidin-4-yloxy)-benzamide

Following General Procedure C, 0.7 g of desired product are obtained in TFA salt form, from the compound of the preceding step.

### Preparation 124

### N-(4-{4-[3-(1-Ethyl-propyl)-ureido]-2-methoxymethyl-phenoxy}-phenyl)-4-(Piperidin-4-yloxy)-benzamide

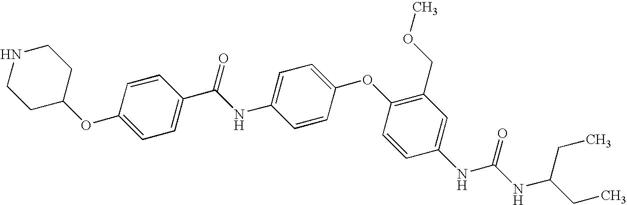

### A/ 4-(1-Benzyl-Piperidin-4-yloxy)-N-(4-{4-[3-(1-ethyl-propyl)-ureido]-2-methoxymethyl-phenoxy}-phenyl)-benzamide

Following General Procedure L1, 1.2 g of 4-(1-Benzyl-Piperidin-4-yloxy)-benzoic acid (Preparation 11) are reacted with 1.42 g of 1-[4-(4-Amino-phenoxy)-3-methoxymethyl-phenyl]-3-(1-ethyl-propyl)-urea (Preparation 81). After evaporation in vacuo, the residue is redissolved in water, extracted with DCM, and the organic layer is washed with an aqueous NaOH solution, dried over MgSO₄, filtered and evaporated. 1 g of product is obtained in the form of a white solid.

### B/ N-(4-{4-[3-(1-Ethyl-propyl)-ureido]-2-methoxymethyl-phenoxy}-phenyl)-4-(Piperidin-4-yloxy)-benzamide

Following General Procedure D, 0.7 g of desired product are obtained from the compound of the preceding step.

### Preparation 125

### N-[4-(4-Amino-2-methoxy-phenoxy)-2-fluoro-phenyl]-4-(1-butyl-Piperidin-4-yloxy)-benzamide

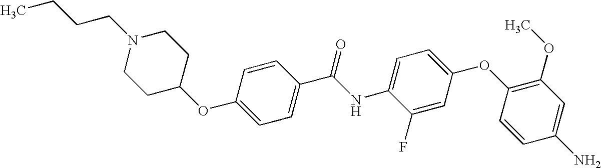

### A/ 4-(1-Butyl-Piperidin-4-yloxy)-N-[2-fluoro-4-(2-methoxy-4-nitro-phenoxy)-phenyl]-benzamide

2.26 g of 4-(1-Butyl-Piperidin-4-yloxy)-benzoic acid (Preparation 5) in 200 ml of DCM are stirred at AT for 1 h in the presence of TBTU (1.3 eq), HOBT (1.3 eq) and DIEA (3 eq), washed with a dilute aqueous NaOH solution, with a dilute aqueous HCl solution, and the organic layer is dried over MgSO₄, filtered and evaporated. 2.46 g of 2-Fluoro-4-(2-methoxy nitro-phenoxy)-phenylamine (Preparation 107, étape A) in DMF are added, concentrated in vacuo at 60° C., the mixture held in vacuo at 60° C. for 3 h and at AT for 72 h. 4 g of desired product are obtained after chromatography on silica eluting with a DCM/MeOH/NH₄OH mixture (98:2:0.1 v/v/v).

### B/ N-[4-(4-Amino-2-methoxy-phenoxy)-2-fluoro-phenyl]-4-(1-butyl-Piperidin-4-yloxy)-benzamide

Following General Procedure E, 0.75 g of desired product are obtained from 1 g of compound of the preceding step.

### Preparation 126

### N-[4-(4-Amino-2-methoxy-phenoxy)-phenyl]-4-(1-butyl-Piperidin-4-yloxy benzamide

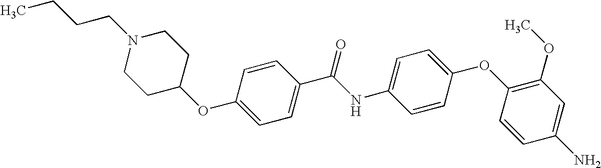

### A/ 4-(1-Butyl-Piperidin-4-yloxy)-N-[4-(2-methoxy-4-nitro-phenoxy)-phenyl]-benzamide

Following the protocol described under Preparation 118, step A, 0.5 g of 4-(1-Butyl-Piperidin-4-yloxy)-benzoic acid (Preparation 5) are reacted with 0.43 g of 4-(2-Methoxy-4-nitro-phenoxy)-phenylamine (Preparation 122, step A). 0.9 g of desired product are obtained after chromatography on silica eluting with a DCM/MeOH mixture (94:6 v/v).

### B/ N-[4-(4-Amino-2-methoxy-phenoxy)-phenyl]-4-(1-butyl-Piperidin-4-yloxy)-benzamide

Under AP and AT, the compound of the preceding step in solution in THF is treated with hydrogen, in the presence of 10% palladium on charcoal. On completion of the reaction, the catalyst is filtered and the solvent partly evaporated. The product obtained in solution is used as such.

### Preparation 127

### N-(8-Amino-10,11-dihydro-dibenzo[b,f]oxepin-2-yl)-4-(1-butyl-piperidin-4-yloxy)-benzamide

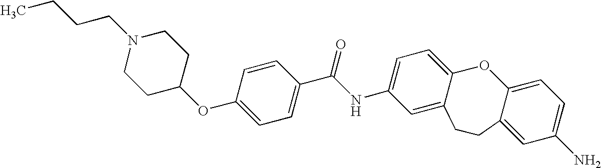

### A/ 5-Nitro-3H-benzofuran-2-one

To a mixture of 60 ml nitric acid (d=1.41 g/ml) and 57 ml of sulfuric acid are added dropwise and at 5° C. 42.9 g of 2-oumaranone solubilized in 73 ml of actiec acid, stirred 10 min at 5° C., then ice and water are added, the formed crystals are drained and washed with water and TBME. 43 g of desired product are obtained.

### B/ (2-Hydroxy-5-nitro-phenyl)-methyl acetate

90 g of product obtained such as described in the preceding step in 4.5 l of MeOH are stirred 18 h at AT, in the presence of 270 g of amberlyst 15. After filtering, the filtrate is evaporated. 105.5 g of desired product are obtained.

### C/ [5-Nitro-2-(4-nitro-phenoxy)-phenyl]-methyl acetate

Following General Procedure O, 36.6 g of product of the preceding step are reacted with 24.6 g of 4-fluoronitrobenzene and heated 35 h at 90° C. After concentration in vacuo, the residue is redissolved in an aqueous NaOH solution, extracted with TBME, dried over MgSO₄, filtered and evaporated. 6.6 g of desired product are obtained after chromatography on silica eluting with DCM.

### D/ [5-Nitro-2-(4-nitro-phenoxy)-phenyl]-acetic acid

The compound of the preceding step in solution in 300 ml of MeOH is heated in the presence of 1.2 g of NaOH, at 50° C. for 12 h. After evaporation in vacuo, addition of water and washing with DCM the aqueous phase is acidified and the formed crystals are filtered and washed with water. 10.2 g of desired product are obtained containing salts, which is used as such.

### E/ 2,8-Dinitro-11H-dibenzo[b,f]oxepin-10-one

The compound of the preceding step is heated at 170° C. for 1 h in 225 g of PPA, poured onto ice, returned to pH 6 with an aqueous NaOH solution, and the insolubles are filtered. 300 ml of methoxyethanol are added to the filtrate, heated under reflux, the insolubles are filtered followed by evaporation in vacuo. 4.2 g of desired product are obtained after chromatography on silica eluting with DCM.

### F/ 2,8-Dinitro-10,11-dihydro-dibenzo[b,f]oxepin-10-ol

The compound of the preceding step in 200 ml of methoxyethanol is reacted with 0.3 g of KBH₄, at AT for 24 h. After concentration in vacuo, an aqueous HCl solution is added, extracted with TBME, dried over MgSO₄, filtered and evaporated. 3.8 g of desired product are obtained.

### G/ 2,8-Dinitro-dibenzo[b,f]oxepine

1.8 g of compound of the preceding step are heated at 110° C. for 1.5 h in 200 g of PPA. The reaction medium is poured onto ice, the precipitate formed is drained and washed with water. 1.5 g of desired product are obtained.

### H/ 10,11-Dihydro-dibenzo[b,f]oxepine-2,8-diamine

Under AP and AT, 2.3 g of compound obtained as described in the preceding step, in solution in 500 ml of methoxyethanol, are treated with hydrogen in the presence of 1 g of platinum oxide. On completion of the reaction, the catalyst is filtered, the solvent evaporated and the residue crystallized in DCM. 0.5 g of desired product are obtained.

### I/ N-(8-Amino-10,11-dihydro-dibenzo[b,f]oxepin-2-yl)-4-(1-butyl-piperidin-4-yloxy)-benzamide

Following the operating mode described under Preparation 125, step A, 0.345 g of 4-(1-Butyl-Piperidin-4-yloxy)-benzoic acid (Preparation 5) are reacted with 0.1 g of compound of the preceding step. 40 mg of desired product are obtained after chromatography on silica eluting with a DCM/MeOH/NH₄OH mixture (90:10:0.1 v/v/v).

### Preparation 128

### N-[4-(4-Amino-2-methoxy-phenoxy)-phenyl]-N-methyl-(8-methyl-8-aza-bicyclo[3.2.1]oct-(3-endo)-yloxy)-benzamide

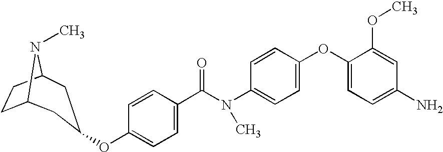

### A/ (3-endo)-{4-[4-(2-Methoxy-4-nitro-phenoxy)-phenylcarbamoyl]-phenoxy}-8-aza-bicyclo[3.2.1]octane-8-tertbutyl carboxylate

Following General Procedure L1, and in 100 ml of DMF, in the presence of HOBT, EDCI and DIEA, 5.9 g of 4-(2-Methoxy-4-nitro-phenoxy)-phenylamine (Preparation 122, step A) are reacted with 6 g of compound obtained such as described under Preparation 120, step C. After evaporation in vacuo, the residue is redissolved in water, the precipitate formed is filtered, washed with water, pentane and with diisopropyl ether. 8.2 g of desired product are isolated, which is used as such.

### B/ (3-endo)-(4-{[4-(2-Methoxy-4-nitro-phenoxy)-phenyl]-methyl-carbamoyl}-phenoxy)-8-aza-bicyclo[3.2.1]octane-8-tertbutyl carboxylate

1.2 g of compound of the preceding step and 89 mg of NaH in 100 ml THF are placed in suspension, stirred 0.5 h at 60° C., 0.5 ml of methyl iodide are added and heating continued at 60° C. for 72 h. After evaporation in vacuo, the residue is redissolved in water, extracted with ethyl acetate, dried over MgSO₄, filtered and evaporated. 1.5 g of desired product are obtained after chromatography on silica eluting with a cyclohexane/ethyl acetate mixture (60:40 v/v).

### C/ 4-(8-Aza-bicyclo[3.2.1]oct-(3-endo)-yloxy)-N-[4-(2-methoxy-4-nitro-phenoxy)-phenyl]-N-methyl-benzamide

The compound of the preceding step is treated following General Procedure C. The reaction medium is evaporated, the residue redissolved in an aqueous NaHCO₃ solution, extracted with DCM, dried over MgSO₄, filtered and evaporated. 1.05 g of desired product are obtained.

### D/ N-[4-(2-Methoxy-4-nitro-phenoxy)-phenyl]-N-methyl-4-(8-methyl-8-aza-bicyclo[3.2.1]oct-(3-endo)-yloxy)-benzamide

The compound of the preceding step is heated under reflux for 5 h in a mixture of formic acid (2 ml)/37% formaldehyde in water (0.6 ml). After concentration in vacuo, the residue is redissolved in water and basified with ammonia, extracted with ethyl acetate, dried over MgSO₄, filtered and evaporated. 0.76 g of desired product are obtained.

### E/ N-[4-(4-Amino-2-methoxy-phenoxy)-phenyl]-N-methyl-4-(8-methyl-8-aza-bicyclo[3.2.1]oct-(3-endo)-yloxy)-benzamide

Following General Procedure E, 0.7 g of desired product are obtained from the compound of the preceding step.

### Preparation 129

### N-[4-(4-Amino-2-methoxy-phenoxy)-phenyl]-4-(8-methyl-8-aza-bicyclo[3.2.1]oct-(3-endo)-yloxy)-N-propyl-benzamide

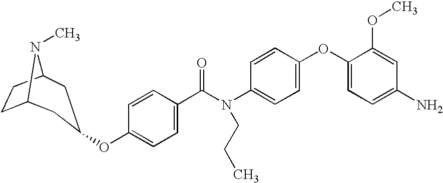

### A/ (3-endo)-(4-{[4-(2-Methoxy-4-nitro-phenoxy)-phenyl]-propyl-carbamoyl}-phenoxy)-8-aza-bicyclo[3.2.1]octane-8-tertbutyl carboxylate

1.5 g of compound obtained such as described under Preparation 128, step A and 110 mg of NaH in 100 ml THF are placed in suspension, stirred 0.5 h at 60° C., 0.24 ml of propyl iodide are added and heated under reflux for 120 h. After evaporation in vacuo, the residue is redissolved in water, extracted with ethyl acetate, dried over MgSO₄, filtered and evaporated. 1.5 g of product are obtained after chromatography on silica eluting with a cyclohexane/ethyl acetate mixture (50:50 v/v). 0.7 g of desired product are obtained.

### B/ 4-(8-Aza-bicyclo[3.2.1]oct-(3-endo-yloxy)-N-[4-(2-methoxy-4-nitro-phenoxy)-phenyl]-N-propyl-benzamide

The desired product is obtained from the compound of the preceding step following the operating mode described under Preparation 128, step C.

### C/ N-[4-(2-Methoxy-4-nitro-phenoxy)-phenyl]-4-(8-methyl-8-aza-bicyclo[3.2.1]oct-(3-endo)-yloxy)-N-propyl-benzamide

The compound of the preceding step is treated following the operating mode described under Preparation 128, step D. 0.3 g of desired product are obtained after chromatography on silica eluting with a DCM/MeOH/NH₄OH mixture (90:10:0.1 v/v/v).

### D/ N-[4-(4-Amino-2-methoxy-phenoxy)-phenyl]-4-(8-methyl-8-aza-bicyclo[3.2.1]oct-(3-endo)-yloxy)-N-propyl-benzamide

Following General Procedure E, 0.2 g of desired product are obtained from the compound of the preceding step.

### Preparation 130

### N-[4-(4-Amino-2-methoxy-phenoxy)-2-fluoro-phenyl]-4-(8-methyl-8-aza-bicyclo[3.2.1]oct-(3-endo)-yloxy)-benzamide

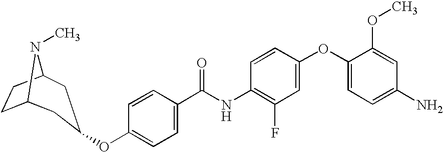

### A/ N-[2-Fluoro-4-(2-methoxy-4-nitro-phenoxy)-phenyl]-4-(8-methyl-8-aza-bicyclo[3.2.1]oct-(3-endo)-yloxy)-benzamide

Following the operating mode described under Preparation 125, step A, 1.47 g of 4-(8-Methyl-8-aza-bicyclo[3.2.1]oct-(3-endo)-yloxy)-benzoic acid (Preparation 27, Method I, step B) are reacted with 0.78 g of 2-Fluoro-4-(2-methoxy-4-nitro-phenoxy)-phenylamine (Preparation 107, step A). 0.57 g of desired product are obtained after chromatography on silica eluting with a DCM/MeOH/NH₄OH mixture (80:20:0.1 v/v/v).

### B/ N-[4-(4-Amino-2-methoxy-phenoxy)-2-fluoro-phenyl]-4-(8-methyl-8-aza-bicyclo[3.2.1]oct-(3-endo)-yloxy)-benzamide

The compound of the preceding step is reacted with 0.63 g of ammonium formiate in MeOH, under nitrogen, in the presence of 5% palladium on charcoal, for 15 h at AT and for 1 h at 50° C. The catalyst is filtered, the solvent evaporated, the residue redissolved in DCM, washed with an aqueous Na₂CO₃ solution, and the organic layer is dried over MgSO₄, filtered and evaporated. 0.43 g of desired product are obtained.

### Preparation 131

### 4-(4-Amino-2-methoxy-phenoxy)-N-[4-(1-butyl-Piperidin-4-yloxy)-phenyl]-benzamide

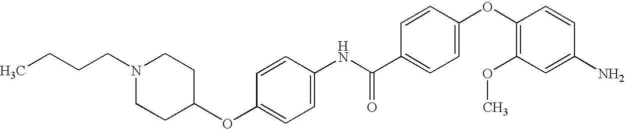

### A/ 4-(2-Methoxy-4-nitro-phenoxy)-ethyl benzoate

Following General Procedure O, 4 g of ethyl-4-hydroxybenzoate are condensed on 4.9 g of 2-chloro-5-nitroanisole. 3.9 g of desired product are isolated.

### B/ 4-(2-Methoxy-4-nitro-phenoxy)-benzoic acid

The compound of the preceding step is treated following General Procedure A. The reaction medium is concentrated, the remaining aqueous solution is washed with TBME, acidified, extracted with DCM, and the last organic layer is dried over MgSO₄, filtered and evaporated. 2.6 g of desired product are obtained.

### C/ 1-Butyl(4-nitro-phenoxy)-Piperidine

To a suspension of 7 g of NaH in DMF (100 ml) are added 20 g of 1-butyl-piperidinol-4-ol (Preparation 3, step A), followed by stirring for 1 h at 40° C., the addition of 14 ml of 4-fluoro-nitrobenzene and stirring for 5 h at 40° C. After evaporating to dryness, the residue is redissolved in an aqueous HCl solution, washed with TBME, the aqueous layer is basified, extracted with DCM, and the last organic layer is dried over MgSO₄, filtered and evaporated. 15.5 g of desired product are isolated after chromatography on silica eluting with a DCM/MeOH mixture (97.5:2.5 v/v).

### D/ 4-(1-Butyl-Piperidin-4-yloxy)-phenylamine

Following General Procedure E, 13 g of desired product are obtained from the compound of the preceding step.

### E/ N-[4-(1-Butyl-Piperidin-4-yloxy)-phenyl]-4-(2-methoxy-4-nitro-phenoxy)-benzamide

Following the operating mode described under Preparation 118, step A, 0.5 g of compound obtained such as described under step B are reacted with 0.43 g of compound of the preceding step. 1.1 g of desired product are obtained after chromatography on silica eluting with a DCM/MeOH mixture (94:6 v/v).

### F/ 4-(4-Amino-2-methoxy-phenoxy)-N-[4-(1-butyl-Piperidin-4-yloxy)-phenyl]-benzamide

The compound of the preceding step, in THF, is treated following General Procedure E. On completion of the reaction, the catalyst is filtered and the solvent partly evaporated. The product obtained in a solution is used as such.

### Preparation 132

### N-[4-(4-Amino-2-methoxy-phenoxy)-2-fluoro-phenyl]-4-(1-butyl-Piperidin-4-yloxy)-3-methyl-benzamide

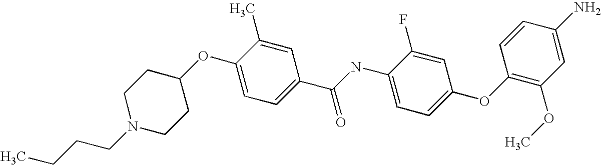

### A/ 4-(1-Butyl-Piperidin-4-yloxy)-N-[2-fluoro-4-(2-methoxy-4-nitro-phenoxy)-phenyl]-3-methyl-benzamide

Following the operating mode described under Preparation 120, step D, 0.75 g of 4-(1-Butyl-Piperidin-4-yloxy)-3-methyl-benzoic acid (Preparation 4) are reacted with 0.48 g of 2-fluoro-4-(2-methoxy-4-nitro-phenoxy)-phenylamine (Preparation 107, step A). 0.33 g of desired product are obtained after chromatography on silica eluting with a DCM/MeOH mixture (98:2 v/v).

### B/ N-[4-(4-Amino-2-methoxy-phenoxy)-2-fluoro-phenyl]-4-(1-butyl-Piperidin-4-yloxy)-3-methyl-benzamide

The desired product is obtained by following General Procedure E to treat the compound of the preceding step.

### Preparation 133

### 3-Methyl-4-(1-methyl-piperidin-4-yloxy)-benzoic acid

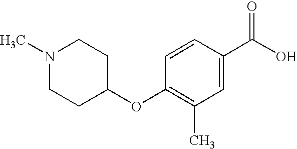

### A/ 3-Methyl-4-(1-methyl-piperidin-4-yloxy)-benzonitrile

In 60 ml of DMF, a mixture of N-methyl-4-hydroxypiperidine (3.8 g), of NaH (1.1 eq) and of 4-fluoro-3-methylbenzonitrile (1 eq) is stirred at AT for 24 h, then evaporated to dryness. The reaction medium is redissolved in water, extracted with DCM and the organic layer is evaporated. The residue is redissolved in TBME, washed with a 1N HCl solution, the aqueous layer is basified, extracted with TBME and the organic layer is dried over MgSO₄, filtered and concentrated. 3 g of desired product are obtained, and used as such.

### B/ 3-Methyl-4-(1-methyl-piperidin-4-yloxy)-benzoic acid

2.6 g of desired product are obtained from the compound of the preceding step by base hydrolysis, following General Procedure B1.

### Preparation 134

### 4-(1-Butyl-piperidin-4-yloxy)-2-methyl-benzoic acid

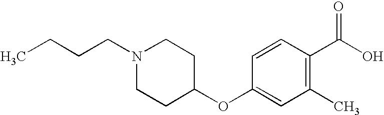

### A/ 4-(1-Butyl-piperidin-4-yloxy)-2-methyl-benzonitrile

In 100 ml of DMF, a mixture of 9.3 g of 1-butyl-piperidin-4-ol (Preparation 3, step A), of NaH (1.3 eq) and of 4-fluoro-2-methylbenzonitrile (1 eq) is stirred at AT for 24 h, then evaporated to dryness. The reaction medium is redissolved in water, extracted with DCM, and the organic layer is dried over MgSO₄, filtered and evaporated. After flash chromatography on silica, eluting with a DCM/MeOH mixture (95:5 v/v), 10.5 g of desired product are isolated.

### B/ 4-(1-Butyl-piperidin-4-yloxy)-2-methyl-benzoic acid

5 g of desired product are obtained from the compound of the preceding step by base hydrolysis, following General Procedure B1.

### Preparation 135

### 4-(1-Butyl-piperidin-4-yloxy)-2-chloro-benzoic acid

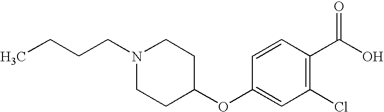

### A/ 4-(1-Butyl-piperidin-4-yloxy)-2-chloro-benzonitrile

In 90 ml of DMF, a mixture of 8.4 g of 1-butyl-piperidinol (Preparation 3, step A), of NaH (1.2 eq) and of 2-chlorofluoro-benzonitrile (1.2 eq) is heated at 80° C. for 12 h, then evaporated to dryness. The reaction medium is redissolved in water, extracted with TBME and the organic layer is dried over MgSO₄, filtered and evaporated. After flash chromatography on silica eluting with a DCM/MeOH mixture (98:2 v/v), 7.5 g of desired product are isolated.

### B/ 4-(1-Butyl-piperidin-4-yloxy)-2-chloro-benzoic acid

2.87 g of desired product are obtained from the compound of the preceding step by base hydrolysis, following General Procedure B1.

### Preparation 136

### 4-(1-Butyl-pyrrolidin-3-yloxy)-benzoic acid

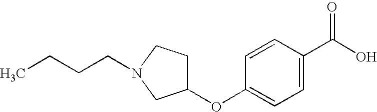

### A/ 1-Butyl-pyrrolidin-3-ol

To a suspension of 3-pyrrolidinol (5 g) and Na₂SO₄ (3 g) in 100 ml DCM, 6.2 ml of butyraldehyde are added and stirred 4 h at AT, then 2 g of sodium triacetoxyborohydride are added slowly and stirring continued for a further 12 h at AT. 100 ml of MeOH are added dropwise and the solvent evaporated in vacuo. After flash chromatography on silica eluting with a DCM/MeOH mixture (95:5 v/v), 1.5 g of desired product are obtained.

### B/ 4-(1-Butyl-pyrrolidin-3-yloxy)-benzonitrile

In 50 ml of DMF, a mixture of 1-butyl-pyrrolidin-3-ol (2.1 g) and NaH (1 eq) is heated at 60° C. for 1 h, then 4-fluorobenzonitrile (1 eq) is added and stirred at AT for 12 h. After evaporating to dryness, the reaction medium is redissolved in water, extracted with ethyl acetate, the organic layer is dried over MgSO₄, filtered and the filtrate evaporated. 980 mg of desired product are isolated after flash chromatography on silica eluting with a DCM/MeOH mixture (98:2 v/v).

### C/ 4-(1-Butyl-pyrrolidin-3-yloxy)-benzoic acid

400 mg of desired product are obtained from the compound of the preceding step by base hydrolysis, following General Procedure B1.

### Preparation 137

### 4-(1-Dimethylamino-piperidin-4-yloxy)-benzoic acid

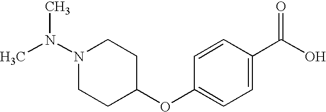

### A/ 3-[N-(2-Ethoxycarbonyl-ethyl)-N′,N′-dimethyl-hydrazino]-ethyl propionate

105 g of ethyl acrylate and 20 g of dimethylhydrazine are heated for 40 h at 100° C. The excess ethyl acrylate is distilled in vacuo, then distilled at 0.1 mmHg (85-100° C.). 50.4 g of desired product are obtained.

### B/ 1-Dimethylamino-piperidin-4-one

5 g of compound obtained in the preceding step are heated to 80° C. with 5.7 g of NaH in 350 ml xylene. The heating is stopped and the remaining 45.4 g of the compound obtained in the preceding step are added, maintaining a small reflux. Then after additional refluxing for one hour, the mixture is cooled, poured onto ice, decanted, 35 ml of concentrated HCl is added and heated under reflux for 4 h until discolouring under the FeCl₃ test. After cooling, basifying with a concentrated NaOH aqueous solution, extracting with DCM and distilling (2 mmHg, 66-70° C.), 11.9 g of desired product are obtained.

### C/ 1-Dimethylamino-piperidin-4-ol

To a solution of 11.9 g of compound obtained in the preceding step in 50 ml THF is added dropwise 1.3 g of LAH in suspension in 50 ml THF. The mixture is stirred 2 h at AT, 50 ml of a saturated Na₂SO₄ solution are added followed by evaporation in vacuo (30 mmHg minimum). 12.4 g of desired product are obtained, which is used as such.

### D/ 4-(1-Dimethylamino-piperidin-4-yloxy)-benzonitrile

In 100 ml DMF, 12.4 g of compound obtained in the preceding step, 3.5 g of NaH and 4-fluorobenzonitrile (10.6 g) are stirred at AT for 7 h. After evaporating to dryness, the reaction medium is redissolved in water, extracted with TBME, washed with a 1N HCl solution. The acid aqueous phase is basified with a concentrated aqueous NaOH solution, extracted with TBME and the organic layer is dried over MgSO₄, filtered and the filtrate evaporated. 9 g of desired product are isolated after crystallization in diisopropyl ether.

### E/ 4-(1-Dimethylamino-piperidin-4-yloxy)-benzoic acid

8.1 g of desired product are obtained from the compound of the preceding step by base hydrolysis, following General Procedure B1.

### Preparation 138

### 4-(1-Butyl-piperidin-4-yloxy)-2,5-difluoro-benzoic acid

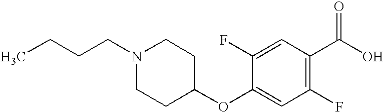

### A/ 4-(4Cyano-2,5-difluoro-phenoxy)-piperidine-1-tertbutyl carboxylate

To a solution of 1-BOC-4-piperidinol (10 g) is added 50 ml of 1 M potassium terbutylate solution and stirred 30 min at AT. This mixture is added to a solution of 2,4,5-trifluorobenzonitrile (1.2 eq) in THF (80 ml) at −65° C., and stirring continued at −65° C. for 3 h and at AT for 12 h. The reaction medium is evaporated to dryness, the residue is redissolved in water, extracted with ethyl acetate, and the organic layer is washed with water and a saturated NaCl solution, dried over MgSO₄, filtered and evaporated. 16.9 g of desired product are obtained, which is used as such.

### B/ 2,5-Difluoro-4-(piperidin-4-yloxy)-benzonitrile

6.9 g of desired product are obtained from the compound of the preceding step by deprotecting the BOC amine, following General Procedure C.

### C/ 4-(1-Butyl-piperidin-4-yloxy)-2,5-difluoro-benzonitrile

A mixture of 6.9 g of product obtained in the preceding step is heated with 3 eq of DIEA and 1-bromobutane (1.2 eq) in 60 ml of DMF for 10 h at 80° C., then evaporated to dryness. After flash chromatography on silica eluting with a DCM/MeOH mixture (95:5 v/v), 3.9 g of desired product are obtained.

### D/ 4-(1-Butyl-piperidin-4-yloxy)-2,5-difluoro-benzoic acid

2.9 g of desired product are obtained from the compound of the preceding step by acid hydrolysis, following General Procedure B2.

### Preparation 139

### 4-(1-Butyl-3-fluoro-piperidin-4-yloxy)-benzoic acid

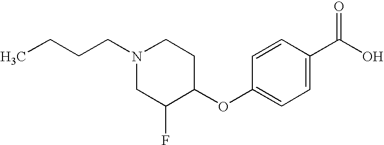

### A/ 4-Trimethylsilanyloxy-3,6-dihydro-2H-pyridine-1-tertbutyl carboxylate

32 ml of TEA are added to a mixture of 19.2 g of 1-BOC-4-piperidone and trimethylsilane chloride (1.2 eq) in 50 ml DMF. The mixture is heated for 11 h at 55° C., a saturated solution of NaHCO₃ is added, followed by extraction with cyclohexane, and drying of the organic layer over MgSO₄, filtering and evaporation. 25.4 g of desired product are obtained, in the form of an orange oil.

### B/ 3-Fluoro-4-oxo-piperidine-1-tertbutyl carboxylate

A solution of 25.4 g of product obtained in the preceding step is stirred 48 h at AT with 36 g of selectfluor in 1 l of acetonitrile. After evaporating to dryness, redissolving in ethyl acetate, washing with a saturated NaCl solution, the organic layer is dried over MgSO₄, filtered and evaporated. After flash chromatography on neutral alumina eluting with an ethyl acetate/MeOH mixture (95:5 v/v), 4.6 g of desired product are obtained.

### C/ 3-Fluoro-4-hydroxy-piperidine-1-tertbutyl carboxylate

3.35 g of NaBH₄ are added in portions to 4.4 g of product obtained in the preceding step in solution in 150 ml of ethanol. The mixture is stirred 24 h at AT, the ethanol concentrated, the residue is redissolved in diethyl ether, washed with water and the organic layer is dried over MgSO₄, filtered and concentrated to dryness. After flash chromatography on neutral alumina eluting with a cyclohexane/ethyl acetate mixture (30:70 v/v), 3 g of desired product are obtained.

### D/ 4-(4Cyano-phenoxy)-3-fluoro-piperidine-1-tertbutyl carboxylate

0.66 g of NaH and 3 g of compound obtained in the preceding step in solution in 50 ml of DMF are heated for 1 h at 50° C. 4-fluorobenzonitrile (1.2 eq) is added and heated for 1 h at 50° C. After return to AT, the solution is poured onto 300 g of ice water, extracted with ethyl acetate, and the organic layer is washed with water, dried over MgSO₄, filtered and the filtrate concentrated to dryness. After chromatography on neutral alumina eluting with a cyclohexane/ethyl acetate mixture (70:30 v/v), 1.4 g of desired product are obtained.

### E/ 4-(3-Fluoro-piperidin-4-yloxy)-benzonitrile

1.4 g of compound obtained in the preceding step in 15 ml DCM are stirred for 48 h at AT with 2 ml of 2N HCl. After filtering, washing with diethyl ether and oven drying, 1.04 g of desired product are obtained, which is used as such.

### F/ 4-(1-Butyl-3-fluoro-piperidin-4-yloxy)-benzonitrile

To a solution of 603 mg of the product obtained in the preceding step, of Na₂SO₄ (1 g), of 429 μl DIEA in 30 ml DCM and of 30 ml acetonitrile, are added 187 mg of butyraldehyde and heated 1.5 h at 45° C., then 747 mg of sodium triacetoxyborohydride are added gradually and stirring continued for 12 h at AT. After washing with a saturated NaHCO₃ solution and with water, the organic layer is dried over MgSO₄, filtered and concentrated. With flash chromatography on silica eluting with a cyclohexane/ethyl acetate mixture (1:1 v/v), 320 mg of desired product are obtained.

### G/ 4-(1-Butyl-3-fluoro-piperidin-4-yloxy)-benzoic acid

376 mg of desired product are obtained from the compound of the preceding step by acid hydrolysis, following General Procedure B2.

### Preparation 140

### 4-[1-(3-Methoxy-propyl)-piperidin-4-yloxy]-3-methyl-benzoic acid

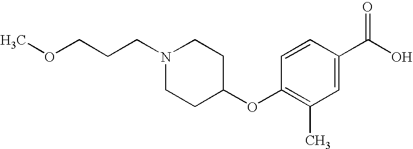

### A/ 3-Methyl-4-(piperidin-4-yloxy)-benzonitrile

A suspension of N—BOC-4-hydroxypiperinide (7 g) and of NaH (1.4 g) in 300 ml DMF is stirred for 30 min. Then 4-chloro-3-methylbenzonitrile (5 g) is added gradually and heated for 5 h at 80° C. After return to AT, water is added, the reaction medium is extracted with diethyl ether, and the organic layer is dried over MgSO₄, filtered and the filtrate concentrated. The residue is redissolved in 150 ml DCM, 10 ml of TFA are added and stirred overnight at AT. The solvent is evaporated, the product precipitated in a mixture of diethyl ether and acetone, and the precipitate filtered. The precipitate is redissolved in a dilute sodium hydroxide solution, extracted with diethyl ether, and the organic layer is dried over MgSO₄, filtered and the filtrate concentrated. 2.9 g of desired product are obtained in the form of a free base.

### B/ 4-[1-(3-Methoxy-propyl)-piperidin-4-yloxy]-3-methyl-benzonitrile

A mixture of the compound obtained as described in the preceding step (5 g), of DIEA (4.5 ml) and of 1-bromo-3-methoxy-propane (3.7 ml) in 200 ml acetonitrile is heated under reflux for 8 h. After return to AT, the solvent is concentrated, the residue redissolved in a dilute sodium hydroxide solution, extracted with TBME, the organic layer is dried over MgSO₄, filtered and the filtrate concentrated. 3.7 g of desired product are obtained.

### C/ 4-[1-(3-Methoxy-propyl)-piperidin-4-yloxy]-3-methyl-benzoic acid

3 g of desired product are obtained from the compound of the preceding step by base hydrolysis, following General Procedure B1.

### Preparation 141

### 4-(1-Butyl-piperidin-4-yloxy)-2-fluoro-5-methyl-benzoic acid

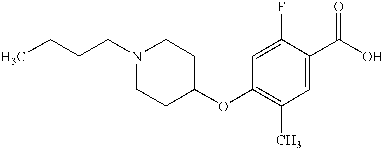

### A/ 4-(5-Fluoro-2-methyl-phenoxy)-piperidine-1-tertbutyl carboxylate

18 g of DIAD are added dropwise to a solution of 10 g of N—BOC-4-hydroxypiperinide, 8.4 g of 2-fluoro-5-methylphenol and 23 g of triphenylphosphine in 300 ml THF, keeping the temperature of the medium to below 40° C. After stirring 12 h at AT, then concentrating, the residue is redissolved in diethyl ether, washed with water and with a 1 N sodium hydroxide solution, the organic layer is dried over MgSO₄, filtered and concentrated to dryness. After chromatography on silica eluting with DCM, 8 g of desired product are obtained in the form of a colourless oil.

### B/ 4-(5-Fluoro-4-iodo-2-methyl-phenoxy)-piperidine

At 0° C., 6 g of succinimide iodide are added to a solution of 8 g of compound obtained in the preceding step in 50 ml TFA. The mixture is stirred 12 h at ambient temperature, concentrated, redissolved in TBME, washed with a 1 N sodium hydroxide, dried over MgSO₄, filtered and concentrated. The residue is redissolved in acetone, hydrochloric ether is added, and the solid that is formed is filtered and washed with diethyl ether. 5.3 g of desired product are obtained in the form of a pale yellow powder.

### C/ 1-Butyl-4-(5-fluoro-4-iodo-2-methyl-phenoxy)-piperidine

To a solution of 4.3 g of compound obtained in the preceding step and 2 ml of DIEA in 50 ml of DCM is added 1.3 ml of butyraldehyde and stirred 15 min at AT. Then 4.8 g of sodium triacetoxyborohydride are added gradually and stirring continued for a further 12 h at AT. After washing with a saturated NaHCO₃ solution and with water, the organic layer is dried over MgSO₄, filtered and concentrated to dryness. 4 g of desired product are obtained, which is used as such.

### D/ 4-(1-Butyl-piperidin-4-yloxy)-2-fluoro-5-methyl-benzonitrile

900 mg of copper cyanide and 4 g of compound obtained in the preceding step in solution in 40 ml DMF are heated under reflux for 6 h. After pouring onto a mixture of water and NH₄OH, and extracting with diethyl ether, the black insoluble formed is filtered and the organic layer is dried over MgSO₄ and concentrated to dryness. 2.4 g of desired product are obtained, which is used as such.

### E/ 4-(1-Butyl-piperidin-4-yloxy)-2-fluoro-5-methyl-benzoic acid

1 g of desired product is obtained from the compound of the preceding step by base hydrolysis, following General Procedure B1.

### Preparation 142

### 4-(4-Butyl-piperazin-1-yl)-benzoic acid

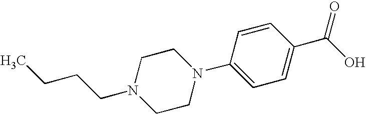

### A/ 4-(4-Cyano-phenyl)-piperazine-1-tertbutyl carboxylate

A solution of 1 g of 4-fluorobenzonitrile, of N—BOC-piperazine (1 eq) and of K₂CO₃ (1.5 eq) in 20 ml DMSO is heated for 48 h at 100° C. Water is added, the precipitate formed is filtered and oven dried. 2 g of desired product are obtained in the form of a white powder.

### B/ 4-piperazin-1-yl-benzonitrile

1.15 g of desired product are obtained from the compound of the preceding step, following General Procedure C.

### C/ 4-(4-Butyl-piperazin-1-yl)-benzonitrile

To a solution of 1.08 g of compound obtained in the preceding step and 1.3 ml TEA in 30 ml DCM, are added 360 mg of butyraldehyde and stirred 5 min at AT. Then 1.44 g of sodium triacetoxyborohydride are added gradually and stirring continued for 1.5 h at AT. After adding a saturated Na₂CO₃ solution, extracting with ethyl acetate and washing with water, the organic layer is dried over Na₂SO₄, filtered and concentrated to dryness. 1.1 g of desired product are obtained, which is used as such.

### D/ 4-(4-Butyl-piperazin-1-yl)-benzoic acid

1 g of desired product is obtained from the compound of the preceding step by acid hydrolysis, following General Procedure B2.

### Preparation 143

### 4-(4-Butyl-[1,4]diazepan-1-yl)-2,5-difluoro-benzoic acid

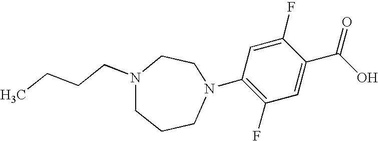

### A/ 4-(4-Cyano-2,5-difluoro-phenyl)-[1,4]diazepane-1-tertbutyl carboxylate

A solution of 10 g of 2,4,5-trifluorobenzonitrile, N—BOC-homopiperazine (12.8 g) and of K₂CO₃ (13.3 g) in 250 ml DMSO is heated for 3 h at 60° C. 500 ml of water are added, the precipitate formed is filtered, redissolved in a mixture of ethyl acetate and methanol, and evaporated. 20 g of desired product are obtained in the form of a yellowish powder.

### B/ 4-[1,4]Diazepan-1-yl-2,5-difluoro-benzonitrile

9.4 g of desired product are obtained from the compound of the preceding step, following General Procedure C.

### C/ 4-(4-Butyl-[1,4]diazepan-1-yl)-2,5-difluoro-benzonitrile

To a solution of 9.4 g of compound obtained in the preceding step and 10.3 ml TEA in 300 ml DCM, are added 3.29 ml of butyraldehyde and stirred for 5 min at AT. Then 10.8 g of sodium triacetoxyborohydride are gradually added and stirring continued 1.5 h at AT. A saturated Na₂CO₃ solution is added, extraction made with ethyl acetate, washed with water, and the organic layer is dried over Na₂SO₄, filtered and concentrated to dryness. 6.2 g of desired product are obtained in the form of a yellow oil.

### D/ 4-(4-Butyl-[1,4]diazepan-1-yl)-2,5-difluoro-benzoic acid

The compound of the preceding step is treated following General Procedure B2. The product obtained is solubilized in 1 N sodium hydroxide, washed with ethyl acetate, the aqueous phase is acidified with a concentrated hydrochloric acid solution, and the precipitate is filtered and dried. 500 mg of desired product are obtained.

### Preparation 144

### 4-(4-Butyl-[1,4]diazepan-1-yl)-benzoic acid

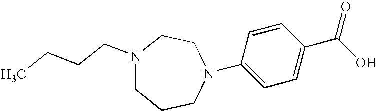

### A/ 4-(4-Methyl-[1,4]diazepan-1-yl)-benzonitrile

Method 1: A solution of 5 g of 4-fluorobenzonitrile, of N-methyl-homopiperazine (5.1 ml) and of K₂CO₃ (1.5 eq) in 60 ml DMF, is heated for 8 h at 140° C. The reaction medium is poured onto ice, the precipitate formed is filtered, the aqueous layer is extracted with ethyl acetate, and the organic layer dried over MgSO₄ and evaporated to dryness. By grouping together the product extracted from the aqueous layer and the formed precipitate, 5.7 g of desired product are obtained in the form of a pinkish-beige powder.

Method 2: A solution of 2.1 g of 4-fluorobenzonitrile, of N-methyl-homopiperazine (1 eq) and of Cs₂CO₃ (1.5 eq) in 20 ml DMSO, is heated for 7 h at 80° C. The reaction medium is poured onto ice, the precipitate formed is filtered, washed with water and oven dried. 2.09 g of desired product are obtained.

### B/ 4-[1,4]Diazepan-1-yl-benzonitrile

A solution of 4.84 g of compound obtained in the preceding step, 11.2 ml of 1-chloroethylchloroformate and 14.1 g of K₂CO₃ in 100 ml DCE, is stirred for 12 h at AT. After filtering, the insoluble is washed with DCM and the organic layer concentrated to dryness. 100 ml of methanol are slowly added to the 7.8 g of product obtained, stirred 4 h at AT and the insoluble filtered. 3.5 g of desired product are obtained and used as such.

### C/ 4-(4-Butyl-[1,4]diazepan-1-yl)-benzonitrile

To a solution of 3.26 g of compound obtained in the preceding step, 2.25 ml of DIEA in 60 ml DCM and 80 ml ACN, is added 1 eq of butyraldehyde and the mixture heated for 1.5 h at 40° C. Next, 4.35 g of sodium triacetoxyborohydride are added gradually and stirring continued for 12 h at AT. After washing with a saturated NaHCO₃ solution then with water, the organic layer is dried over MgSO₄, filtered and concentrated to dryness. After flash chromatography on silica eluting with a cyclohexane/ethyl acetate mixture (1:1 v/v), 1.65 g of desired product are obtained.

### D/ 4-(4-Butyl-[1,4]diazepan-1-yl)-benzoic acid

480 mg of desired product are obtained from the compound of the preceding step by acid hydrolysis, following General Procedure B2.

### Preparation 145

### 4-(4-Methyl-[1,4]diazepan-1-yl)-benzoic acid

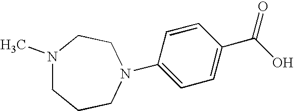

4.4 g of desired product are obtained by base hydrolysis, following General Procedure B1, from 5.05 g of 4-(4-methyl-[1,4]diazepan-1-yl)-benzonitrile obtained such as described under Preparation 144, step A.

### Preparation 146

### 4-(4-ethyl-piperazin-1-yl)-benzoic acid

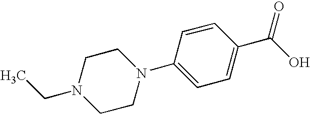

### A/ 4-(4-Ethyl-piperazin-1-yl)-ethyl benzoate

A solution of 1-ethylpiperazine (14.7 ml) and ethylfluorobenzoate (14.7 ml) in 110 ml DMF, is heated for 12 h at 80° C., then evaporated to dryness. After flash chromatography on silica, eluting with a DCM/MeOH/NH₄OH mixture (90:10:1 v/v/v), 6.5 g of desired product are obtained.

### B/ 4-(4-Ethyl-piperazin-1-yl)-benzoic acid

6.5 g of compound obtained in the preceding step are heated under reflux for 4 h with 50 ml of 37% HCl and 100 ml of water. After evaporating to dryness, the residue is redissolved in a mixture of diethyl ether and DCM, filtered, washed with methanol and oven dried. 1.8 g of desired product are obtained in the form of a grey powder.

### Preparation 147

### 4-(4Butyl-piperazin-1-yl)-2-fluoro-5-methyl-benzoic acid

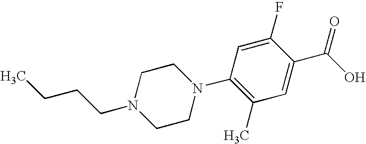

### A/ 1-(5-Fluoro-2-methyl-phenyl)-piperazine

A solution of 25 g of 3-fluoro-5-methylaniline and bis(2-chloroethyl)amine (39 g) in xylene, is heated under reflux for 16 h. After hot filtration and washing with acetone, the solid is redissolved in a dilute sodium hydroxide solution, extracted with ethyl acetate, dried over MgSO₄, filtered and concentrated. The residue is redissolved in diisopropyl ether, and the precipitate is filtered. 4.5 g of desired product are obtained.

### B/ 1-Butyl(5-fluoro-2-methyl-phenyl)-piperazine

To a solution of 4.5 g of compound obtained in the preceding step, in 100 ml DCM, is first added butyraldehyde (2.5 ml) then gradually 7 g of sodium triacetoxyborohydride followed by stirring for 6 h at AT. After washing with a saturated NH₄OH solution then with water, the organic layer is dried over MgSO₄, filtered and concentrated to dryness. 3.4 g of desired product are obtained.

### C/ 1-Butyl-4-(5-fluoro-4-iodo-2-methyl-phenyl)piperazine

At 0° C., 3.2 g of succinimide iodide are added to a solution of 3.4 g of the compound obtained in the preceding step in 20 ml TFA. After stirring 12 h at AT, the mixture is concentrated, redissolved in TBME, washed with 1 N sodium hydroxide then with a saturated NaHCO₃ solution, dried over MgSO₄, filtered and concentrated. 4.1 g of desired product are obtained in the form of a pale yellow powder.

### D/ 4-(4-Butyl-piperazin-1-yl)-2-fluoro-5-methyl-benzonitrile

1 g of copper cyanide and 4.1 g of compound obtained in the preceding step in solution in 50 ml DMF, are heated under reflux for 6 h. Then, after pouring into a mixture of water and NH₄OH and extracting with TBME, the organic layer is dried over MgSO₄ and concentrated to dryness. 2.7 g of desired product are obtained, which is used as such.

### E/ 4-(4-Butyl-piperazin-1-yl)-2-fluoro-5-methyl-benzoic acid

1.4 g of desired product are obtained from the compound of the preceding step by base hydrolysis, following General Procedure B1

### Preparation 148

### 4-(4-Ethyl-piperazin-1-ylmethyl)-benzoic acid

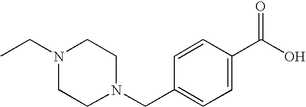

### A/ 4-(4-Ethyl-piperazin-1-ylmethyl)-methyl benzoate

A solution of 11.7 g of 4-bromomethyl-methyl benzoate, of N-ethylpiperazine (1.1 eq) and 14 g of K₂CO₃ in 70 ml ethanol, is heated for 12 h at 80° C. After concentrating to dryness, the residue is redissolved in DCM, washed with water, dried over MgSO₄ and concentrated to dryness. After flash chromatography on silica eluting with a DCM/MeOH mixture (95:5 v/v), 11 g of desired product are obtained.

### B/ 4-(4-Ethyl-piperazin-1-ylmethyl)-benzoic acid

10 g of desired product are isolated from the compound of the preceding step by saponifying the ester in accordance with General Procedure A.

### Preparation 149

### 1-(1-Methyl-piperidin-4-yl)-1H-indole-5-carboxylic acid

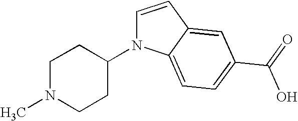

### A/ 3-(2-Dimethylamino-vinyl)-4-nitro-benzoate methyl ester

A solution of 20 g of 3-methyl-4-nitro-methyl benzoate and 34 ml of dimethylformamide dimethylacetal in 220 ml DMF, is heated for 18 h at 140° C. After concentrating to dryness, the residue is redissolved in 140 ml of methanol. The product crystallizes at 0° C., it is filtered and washed with MeOH and with pentane. 15.2 g of desired product are obtained.

### B/ 3-(2,2-Dimethoxy-ethyl)-4-nitro-methyl benzoate

A solution of 15.2 g of compound obtained in the preceding step and of chlorotrimethylsilane (19.3 ml) in 200 ml methanol, is heated under reflux for 18 h. After concentration, the residue is dissolved in TBME, washed with water, with a saturated NaHCO₃ solution, then with water. The organic layer is dried over MgSO₄, filtered and evaporated. 10.5 g of desired product are obtained.

### C/ 4-Amino-3-(2,2-dimethoxy-ethyl)methyl benzoate

10.5 g of compound obtained in the preceding step, in solution in 600 ml of methanol, is treated with hydrogen in the presence of a catalytic quantity of 10% Pd/C. The catalyst is filtered, washed with methanol and the solvent concentrated. 9.9 g of desired product are obtained, which is used as such.

### D/ 3-(2,2-Dimethoxy-ethyl)-4-(1-methyl-piperidin-4-ylamino)-methyl benzoate

A solution of 9.9 g of compound obtained in the preceding step, of N-methyl-4-piperidone (1 eq) and of Na₂SO₄ (62 g) in 208 ml of acetic acid, is stirred for 15 min AT. Then 26.3 g of sodium triacetoxyborohydride are added gradually and stirring continued for 1 h at AT. The mixture is next poured into 600 ml of a saturated aqueous NaHCO₃ solution, extracted with TBME and the organic layer is dried over MgSO₄, filtered and concentrated to dryness. 12.7 g of desired product are obtained, which is used as such.

### E/ 1-(1-Methyl-piperidin-4-yl)-1H-indole-5-methyl carboxylate

12.7 g of compound obtained in the preceding step in 250 ml of 1.6 N hydrochloric methanol are heated under reflux for 1.5 h. After evaporation, the residue is redissolved in ice water, washed with TBME, the aqueous layer is basified and extracted with DCM, dried over MgSO₄ and concentrated to dryness. The residue is redissolved in a mixture of diisopropyl ether and TBME, the precipitate formed is filtered, the organic layer concentrated and purified by chromatography on silica eluting with a DCM/MeOH mixture (95:5 v/v). 7.1 g of desired product are obtained.

### F/ 1-(1-methyl-piperidin-4-yl)-1H-indole-5-carboxylic acid

7 g of desired product are isolated from the compound of the preceding step by saponification, following General Procedure A.

### Preparation 150

### 1-(1-Butyl-piperidin-4-yl)-1H-indole-5-carboxylic acid

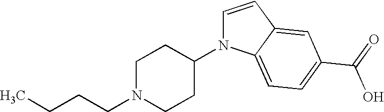

### A/ 4-(1-Butyl-piperidin-4-ylamino)-3-(2,2-dimethoxy-ethyl)-methyl benzoate

A solution of 10.4 g of compound obtained under Preparation 149, step C, of Na₂SO₄ (65 g) and of N-butylpiperidinone (7.1 g) in 220 ml acetic acid, is stirred 15 min at AT. 27.6 g of sodium triacetoxyborohydride are gradually added and stirring continued 1 h at AT. Then, after pouring into 700 ml of a saturated NaHCO₃ solution, extracting with TBME, the organic layer is dried over MgSO₄, filtered and concentrated to dryness. 13.8 g of desired product are obtained, which is used as such.

### B/ 1-(1-Butyl-piperidin-4-yl)-1H-indole-5-methyl carboxylate

13.8 g of compound obtained in the preceding step in 250 ml of 1.6 N hydrochloric methanol are heated for 1.5 h under reflux. After evaporation, the residue is redissolved in iced water, washed with TBME, dried over MgSO₄ and concentrated to dryness. 7.5 g of desired product are obtained after chromatography on silica eluting with a DCM/MeOH mixture (95:5 v/v).

### C/ 1-(1-Butyl-piperidin-4-yl)-1H-indole-5-carboxylic acid

5.4 g of desired product are isolated from the compound of the preceding step by saponification, following General Procedure A.

### Preparation 151

### 1-(1-Butyl-pipéridin-4-yl)-2,3-dihydro-1H-indole-5-carboxylic acid

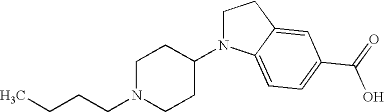

### A/ 1-(1-Butyl-piperidin-4-yl)-2,3-dihydro-1H-indole-5-methyl carboxylate

2 g of sodium cyanoborohydride are gradually added to a solution of 1.3 g of the compound obtained in step B of Preparation 150, in 20 ml of acetic acid, and stirred 60 h at AT. This solution is poured onto a mixture of ice and sodium hydroxide, extracted with TBME, dried over MgSO₄, concentrated to dryness, and the residue is purified by semi-preparative HPLC. 300 mg of desired product are obtained.

### B/ 1-(1-Butyl-piperidin-4-yl)-2,3-dihydro-1H-indole-5-carboxylic acid

300 mg of the compound obtained in the preceding step are heated under reflux for 8 h with 10 ml of 37% hydrochloric acid and 10 ml of water. After evaporating to dryness, the residue is redissolved in a mixture of water/acetone/ACN, filtered, washed with acetone, and oven dried. 43 mg of desired product are obtained.

### Preparation 152

### 2-Methyl-1,2,3,4-tetrahydro-benzo[4,5]furo[3,2-c]pyridine-8-carboxylic acid

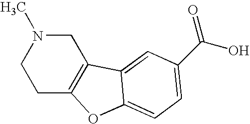

### A/ 1-Methyl-piperidin-4-one oxime

73 g of hydroxylamine hydrochloride are gradually added to a solution of sodium acetate (108.7 g) and N-methyl-piperidone (100 g) in ethanol (2 l). The mixture is stirred 48 h at AT and filtered through celite. The filtrate is concentrated, the residue redissolved in DCM, washed with a 10% Na₂CO₃ solution, the aqueous layer is extracted with DCM. The organic phases are grouped together, washed with water, dried over MgSO₄, filtered and concentrated to dryness. 62 g of desired product are obtained, which is used as such.

### B/ 4-(1-Methyl-piperidin-4-ylideneaminooxy)-benzonitrile

At 0° C., 1.87 g of NaH are added to a solution de 5 g of compound obtained in the preceding step in 60 ml THF, then 2 eq of ethyl-4-fluorobenzoate in 20 ml DMSO are added and stirred 12 h at AT. The THF is concentrated, excess NaH removed with water, and extraction made with diethyl ether. The organic layer is washed with a 2 M Na₂CO₃ solution, dried over MgSO₄ and evaporated to dryness. After chromatography on alumina eluting with hexane then with DCM, 3.07 g of desired product are obtained.

### C/ 4a-Hydroxy-2-methyl-1,2,3,4,4a,9b-hexahydro-benzo[4,5]furo[3,2-c]pyridine-8-carbonitrile

A solution of 2.01 g of compound obtained in the preceding step and 14 ml of a 1 N HCl solution in dioxane are stirred 48 h at AT. The medium is neutralized with 120 ml of an aqueous 2 M solution of Na₂CO₃, extracted with DCM, and the organic layer is dried over MgSO₄ and evaporated. 2.13 g of desired product are obtained, which is used as such.

### D/ 2-Methyl-1,2,3,4-tetrahydro-benzo[4,5]furo[3,2-]pyridine-8-carbonitrile

A mixture of 1.25 g of compound obtained in the preceding step and 10 ml of triflic acid is stirred 48 h at AT. The solution is poured into 120 ml of a 2 M Na₂CO₃ solution, extracted with DCM, and the organic layer is dried over MgSO₄ and concentrated to dryness. 1.12 g of desired product are obtained, which is used as such

### E/ 2-Methyl-1,2,3,4-tetrahydro-benzo[4,5]furo[3,2-c]pyridine-8-carboxylic acid

A mixture of 2 g of compound obtained in the preceding step, of sodium hydroxide (3 eq), ethanol (3 ml) and water (30 ml) is heated under reflux for 48 h. The ethanol is evaporated, the aqueous layer is acidified with an amberlite resin, filtered, the resin washed with methanol and evaporated. 1.7 g of desired product are obtained in white solid form.

### Preparation 153

### 1-[4-(4-Amino-phenoxy)-2,5-difluoro-phenyl]-3-(1-ethyl-propyl)-urea

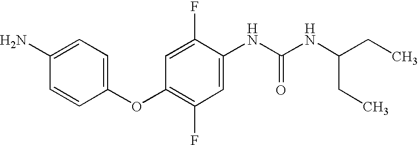

### A/ [4-(2,5-Difluoro-4-nitro-phenoxy)-phenyl]-tertbutyl carbamate

At 5° C., 76 ml of a 1 M solution of potassium terbutylate in THF are added dropwise to a solution of 15.8 g of 4-N—BOC-aminophenol. After stirring 30 min 1,3,4-trifluoronitrobenzene (13.5 g) in solution in 45 ml THF is poured drop by drop at −65° C. and stirred 3 h at −65° C. After adding water and extracting with TBME, the organic layer is washed with water, dried over MgSO₄, filtered and evaporated. After chromatography on silica eluting with a DCM/pentane mixture (50:50 (vv), the product obtained is recrystallized in TBME. 4.2 g of desired product are obtained.

### B/ [4-(4-Amino-2,5-difluoro-phenoxy)-phenyl]-tertbutyl carbamate

Following General Procedure E, 3.7 g of desired product are obtained from 4.2 g of compound obtained in the preceding step.

### C/ (4-{4-[3-(1-Ethyl-propyl)-ureido]-2,5-difluoro-phenoxy}-phenyl)-tertbutyl carbamate

Following General Procedure H, 2 g of desired product are obtained from 3.7 g of compound obtained in the preceding step.

### D/ 1-[4-(4-Amino-phenoxy)-2,5-difluoro-phenyl]-3-(1-ethyl-propyl)-urea

Following General Procedure C, 3.3 g of desired product are obtained in base form from 3.3 g of compound obtained such as described in the preceding step.

### Preparation 154

### 1-[4-(4-Amino-2-methyl-phenoxy)-2-fluoro-phenyl]-3-(1-ethyl-propyl)-urea

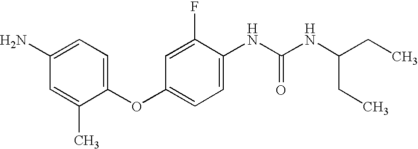

### A/ [2-Fluoro-4-(2-methyl-4-nitro-phenoxy)-phenyl]-tertbutyl carbamate

Following General Procedure O, 10 g of 3-fluoro-4-N—BOC-aminophenol described under step A, Preparation 85 are reacted with 7.5 g of 2-fluoro-5-nitrotoluene. 10.2 g of desired product are isolated after precipitation in an ether/pentane mixture.

### B/ 2-Fluoro-4-(2-methyl-4-nitro-phenoxy)-phenylamine

Following General Procedure C, 6.2 g of desired product are obtained in the form of a free base from the compound obtained in the preceding step.

### C/ 1-(1-Ethyl-propyl)-3-[2-fluoro-4-(2-methyl-4-nitro-phenoxy)-phenyl]-urea

Following General Procedure H, 6 g of desired product are obtained from the compound obtained in the preceding step.

### D/ 1-[4-(4-Amino-2-methyl-phenoxy)-2-fluoro-phenyl]-3-(1-ethyl-propyl)-urea

The compound of the preceding step is treated following General Procedure E. 4 g of desired product are isolated in the form of a free base after precipitation in methanol.

### Preparation 155

### 1-[4-(4-Amino-phenoxy)-2-fluoro-5-methoxy-phenyl]-3-(1-ethyl-propyl)-urea

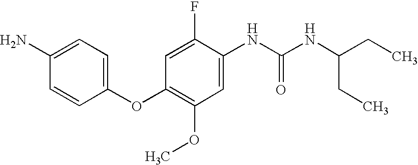

### A/ 5-Fluoro-2-methoxy-phenol

106 ml of 2.5 M butyllithium in hexane are added dropwise at −20° C. to a solution of 2-bromo-4-fluoro-1-methoxy-benzene (50 g) in 1 l of pentane, and stirred for 15 min at −10° C., then cooled to −30° C. Then trimethylborate (30 ml) is added, stirred 30 min at 0° C., cooled to −10° C., followed by the addition of a 32% peracetic solution (103 ml) over 45 min keeping the temperature to below −5° C. and stirring 30 min at 0° C. The mixture is cooled to −10° C., 150 ml of a saturated NaHSO₃ solution are added, stirred 1 h at AT, then after adding water, neutralizing with 330 g of NaHCO₃ and decanting the pentane, the aqueous layer is extracted with DCM. The organic layer is washed with sodium hydroxide, the aqueous layer is acidified with a concentrated HCl solution, extracted with DCM and the organic layer is dried over MgSO₄, filtered and concentrated to dryness. 27.1 g of desired product are obtained.

### B/ 2-Benzyloxy-4-fluoro-1-methoxy-benzene

51 ml of benzyl bromide are added to a solution of 55.2 g of product, obtained as described in the preceding step, and K₂CO₃ (85 g) in acetone (600 ml). After heating under reflux 4 h, concentrating, the residue is redissolved water, extracted with TBME, the organic layer is washed with water, dried over MgSO₄, filtered and evaporated. After precipitating in diisopropyl ether, filtering and drying, 70.1 g of desired product are obtained.

### C/ 1-Benzyloxy-5-fluoro-2-methoxy-4-nitro-benzene

70.1 g of product obtained in the preceding step are added gradually to a 63% solution of nitric acid (140 ml) in 494 ml of acetic acid, keeping the temperature at 25° C. using an iced water bath. After stirring 2 h at AT, the solution is poured into 1 l of ice water, the precipitate is filtered, washed with water and pentane and dried. 77.9 g of desired product are obtained.

### D/ 4-Amino-5-fluoro-2-methoxy-phenol

77.9 g of compound obtained in the preceding step in solution in methoxyethanol are treated with hydrogen under AP and at AT in the presence of a catalytic quantity of palladium on charcoal. After filtering the catalyst, washing with methoxyethanol and concentrating to dryness, the residue is redissolved in TBME, filtered and dried in vacuo at 60° C. 37.1 g of desired product are obtained.

### E/ 2-Fluoro-5-methoxy-4-(4-nitro-phenoxy)-phenylamine

A solution of 4-fluoronitrobenzene (10.8 g), of K₂CO₃ (12 g) and 12 g of product obtained in the preceding step in 400 ml of anhydrous acetone is heated under reflux for 7 days. After concentrating to dryness, the residue is redissolved in TBME, washed with a saturated NaCl solution, the organic layer is dried over Na₂SO₄, filtered and concentrated. 14.9 g of desired product are isolated after flash chromatography on silica eluting with DCM.

### F/ 1-(1-Ethyl-propyl)-3-[2-fluoro-5-methoxy-4-(4-nitro-phenoxy)-phenyl]-urea

9 g of compound obtained in the preceding step are treated following General Procedure H using ACN as reaction solvent. After cold precipitation in the medium, the precipitate is filtered and washed with ACN. 7.2 g of desired product are obtained, which is 80% pure and used as such.

### G/ 1-[4-(4-Amino-phenoxy)-2-fluoro-5-methoxy-phenyl]-3-(1-ethyl-propyl)-urea

The compound obtained in the preceding step is treated following General Procedure E. After flash chromatography on silica eluting with a cyclohexane/ethyl acetate mixture (1:1 v/v), 6.6 g of desired product are obtained.

### Preparation 156

### 1-[4-(4-Amino-phenoxy)-2-fluoro-5-methoxy-phenyl]-3-isopropyl-urea

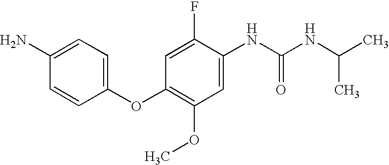

### A/ 1-[2-Fluoro-5-methoxy-4-(4-nitro-phenoxy)-phenyl]-3-isopropyl-urea

A solution of 1 g of compound obtained such as described under step E, Preparation 155, and of isopropyl isocyanate (2.65 ml) in ACN (100 ml) is heated under reflux for 4 days. After concentration, the residue is redissolved in diethyl ether, and the precipitate filtered. 760 mg of desired product are isolated after chromatography on silica eluting with DCM.

### B/ 1-[4-(4-Amino-phenoxy)-2-fluoro-5-methoxy-phenyl]-3-isopropyl-urea

240 mg of desired product are obtained when following General Procedure E to treat the compound obtained in the preceding step, and after chromatography on silica eluting with TBME.

### Preparation 157

### 1-[4-(4-Amino-phenoxy)-2-fluoro-5-methoxy-phenyl]-3-(1-methoxymethyl-propyl)-urea

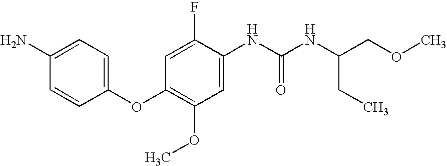

### A/ Imidazole-1-carboxylic acid (1-methoxymethyl-propyl)-amide

1.7 g of desired product are obtained when following General Procedure G, replacing 1-ethyl-propylamine by 1-methoxymethyl-propylamine.

### B/ 1-[2-Fluoro-5-methoxy-4-(4-nitro-phenoxy)-phenyl]-3-(1-methoxymethyl-propyl)-urea

A solution of 1 g of compound obtained such as described under step E, Preparation 155 and the product obtained in the preceding step in 150 ml DMF, is heated 6 h at 140° C. After adding water and filtering, the precipitate is redissolved in acetone and filtered again. Diethyl ether is added to the acetone, washed with water and with a concentrated aqueous 1 N solution of HCl, and concentrated. After two successive crystallizations with diethyl ether and ethyl acetate, 210 mg of desired product are obtained.

### C/ 1-[4-(4-Amino-phenoxy)-2-fluoro-5-methoxy-phenyl]-3-(1-methoxymethyl-propyl)-urea

Following General Procedure E, 200 mg of desired product are obtained from the compound obtained in the preceding step.

### Preparation 158

### 1-[4-(4-Amino-phenoxy)-5-ethoxy-2-fluoro-phenyl]-3-(1-ethyl-propyl)-urea

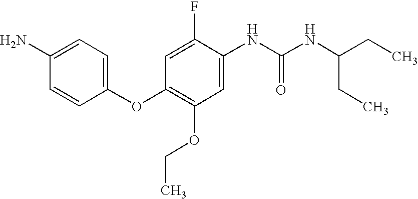

### A/ 5-Amino-4-fluoro-2-(4-nitro-phenoxy)-phenol

A solution of 5.5 g of compound obtained such as described under step E, Preparation 155 in 160 ml of concentrated hydrobromic acid is heated 2.5 h at 150° C. After basifying with an aqueous concentrated sodium hydroxide solution and extracting with ethyl acetate, the organic layer is dried over MgSO₄, filtered and concentrated. 5.1 g of desired product are obtained, which is used as such.

### B/ [2-Fluoro-5-hydroxy-4-(4-nitro-phenoxy)-phenyl]-tertbutyl carbamate

A solution of 4.5 g of product obtained in the preceding step and BOC₂O (1 eq) in 100 ml THF, is heated under reflux for 24 h. The reaction medium is concentrated to dryness and purified by chromatography on silica, eluting with a cyclohexane/ethyl acetate mixture (80:20 v/v). 2.96 g of desired product are obtained.

### C/ [5-Ethoxy-2-fluoro-4-(4-nitro-phenoxy)-phenyl]-tertbutyl carbamate

A suspension of 2.95 g of compound obtained in the preceding step, of K₂CO₃ (1.67 g) and ethyl iodide (0.7 ml) in 100 ml of acetone, is heated 6 h at 60° C. The insoluble is filtered, the filtrate concentrated, the residue redissolved in ethyl acetate, washed with water and the organic layer is dried over MgSO₄, filtered and concentrated to dryness. After flash chromatography on silica eluting with a cyclohexane/ethyl acetate mixture (90:10 v/v), 1.6 g of desired product are obtained.

### D/ 5-Ethoxy-2-fluoro-4-(4-nitro-phenoxy)-phenylamine

1.2 g of desired product are isolated by following General Procedure C to treat the compound obtained in the preceding step.

### E/ 1-[5Ethoxy-2-fluoro-4-(4-nitro-phenoxy)-phenyl]-3-(1-ethyl-propyl)-urea

Following General Procedure H, 1.1 g of desired product are isolated after treating the compound obtained in the preceding step.

### F/ 1-[4-(4-Amino-phenoxy)-5-ethoxy-2-fluoro-phenyl]-3-(1-ethyl-propyl)-urea

Following General Procedure E, 980 mg of desired product are isolated after treating the compound obtained in the preceding step.

### Preparation 159

### 1-[4-(4-Amino-3-fluoro-phenoxy)-3-(2-dimethylamino-ethoxy)-phenyl]-3-(1-ethyl-propyl)-urea

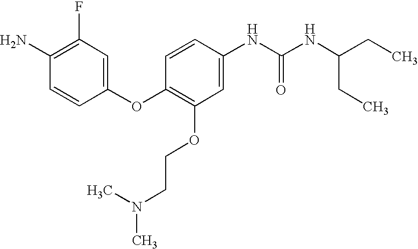

### A/ {4-[2-(2-Dimethylamino-ethoxy)-4-nitro-phenoxy]-2-fluoro-phenyl}-tertbutyl carbamate

A suspension of 1.5 g of compound obtained such as described under step B, Preparation 157, of K₂CO₃ (2.5 eq), of N,N-dimethylchloroethylamine chloride (1.1 eq) and a catalytic quantity of potassium iodide in 20 ml DMF, is heated 2 h at 60° C. After adding ethyl acetate, washing with water, the organic layer is dried over MgSO₄, filtered and concentrated to dryness. Then, after precipitation in diisopropyl ether and filtration, 950 mg of desired product are obtained.

### B/ {4-[4-Amino-2-(2-dimethylamino-ethoxy)-phenoxy]-2-fluoro-phenyl}-tertbutyl carbamate

950 mg of compound obtained in the preceding step in solution in THF are treated with hydrogen under AP and at AT in the presence of 10% palladium on charcoal. After filtering the catalyst, washing with THF, the organic solvent is concentrated to dryness. 800 mg of desired product are obtained.

### C/ [2-Fluoro-4-(2-hydroxy-4-nitro-phenoxy)-phenyl]-tertbutyl carbamate

445 mg of desired product are isolated by following General Procedure H to treat the compound obtained in the preceding step.

### D/ 1-[4-(4-Amino-3-fluoro-phenoxy)-3-(2-dimethylamino-ethoxy)-phenyl]-3-(1-ethyl-propyl)-urea

400 mg of desired product are obtained in TFA salt form by following General Procedure C to treat the compound obtained in the preceding step.

### Preparation 160

### (1-Ethyl-propyl)-carbamate of 4-(4-amino-phenoxy)-3-methoxy-phenyl

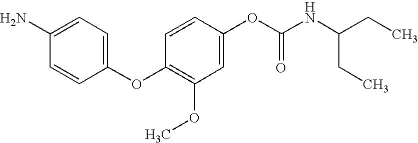

### A/ 4-Benzyloxy-2-methoxy-phenol

A solution of 2-methoxyhydroquinone (14.5 g) in 200 ml DMF is heated 1 h at 165° C., then benzyl chloride (11.98 ml) is added gradually and heated 1.5 h at 165° C. The DMF is evaporated, the residue redissolved in DCM, washed with water and the organic layer is dried over MgSO₄, filtered and the filtrate evaporated. After flash chromatography on silica eluting with a cyclohexane/ethyl acetate mixture (9:1 v/v), 1.1 g of desired product are obtained.

### B/ 4-Benzyloxy-2-methoxy-1-(4-nitro-phenoxy)-benzene

The product obtained such as described in the preceding step is reacted 12 h at AT with 4-fluoronitrobenzene, following General Procedure O. After evaporating the DMF, the reaction medium is redissolved in TBME, washed with a 1 N sodium hydroxide solution, and the organic layer is dried over MgSO₄, filtered and concentrated to dryness. The residue is redissolved in pentane, filtered and dried. 1.6 g of desired product are obtained.

### C/ 4-(4-Amino-phenoxy)-3-methoxy-phenol

1.6 g of compound obtained in the preceding step in solution in THF (150 ml) are treated with hydrogen under AP and at AT in the presence of a catalytic quantity of palladium on charcoal. The catalyst is filtered followed by washing with methanol, and the organic solvent is concentrated to dryness 1.04 g of desired product are obtained.

### D/ [4-(4-Hydroxy-2-methoxy-phenoxy)-phenyl]-tertbutyl carbamate

General Procedure F is followed to treat the compound obtained in the preceding step. After THF evaporation, purification is conducted by chromatography on silica eluting with a cyclohexane/ethyl acetate mixture (8:2 v/v). 639 mg of desired product are obtained.

### E/ {4-[4-(1-Ethyl-propylcarbamoyloxy)-2-methoxy-phenoxy]-phenyl}-tertbutyl carbamate

A solution of 657 mg of product obtained such as described in the preceding step and of TEA (3 eq) in 4 ml THF and 5 ml DCM is added gradually at −10° C. to a solution of chloromethyl chloroformate in 5 ml THF. The reaction medium is stirred 3 h and left to return to AT. 3-aminopentane (4 eq) and TEA (1 ml) are added and stirred for 12 h at AT, then heated under reflux for 3 h after which the reaction medium is concentrated to dryness. The residue is redissolved in DCM, washed with a 1 N NaOH solution and the organic layer is dried over MgSO₄, filtered and the filtrate evaporated. After chromatography on silica eluting with a DCM/MeOH mixture (99:1 v/v), 212 mg of desired product are obtained.

### F/ (1-Ethyl-propyl)-carbamate of 4-(4-amino-phenoxy)-3-methoxy-phenyl

General Procedure C is followed to treat the compound obtained such as described in the preceding step. After concentrating the reaction medium, the residue is redissolved in DCM, a saturated Na₂CO₃ solution is added, the aqueous layer extracted with DCM, and the organic layer is washed with water, dried over MgSO₄, filtered and the filtrate concentrated. 200 mg of desired product are obtained.

### Preparation 161

### 1-(1-Ethyl-propyl)-3-{2-fluoro-5-methoxy-4-[4-(2-methoxy-ethylamino)-phenoxy]-phenyl}-urea

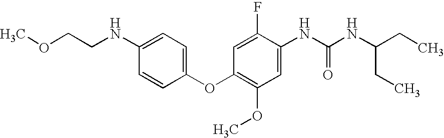

A solution of 1.26 g of compound obtained as described under Preparation 155, of DIEA (1.1 eq) and 2-bromoethylmethylether in 20 ml DMF is heated 48 h at 80° C. The solvent is evaporated, the reaction medium redissolved in water, filtered, the residue redissolved in DCM, washed with water and the organic layer is dried over MgSO₄ and concentrated dry. 507 mg of desired product are obtained in the form of a beige powder after flash chromatography on silica eluting with a DCM/MeOH mixture (97:3 v/v).

### Preparation 162

### 1-[4-(4-Ethylamino-phenoxy)-2-fluoro-5-methoxy-phenyl]-3-(1-ethyl-propyl)-urea

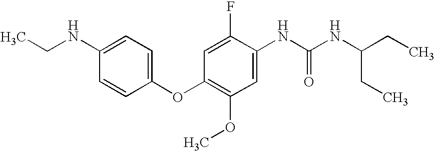

### A/ N-(4-{4-[3-(1-Ethyl-propyl)-ureido]-5-fluoro-2-methoxy-phenoxy}-phenyl)acetamide

To a solution in acetic acid (1 ml) of compound obtained such as described under Preparation 155 (200 mg) is added acetic anhydride (1 ml) and stirred 12 h at AT. The reaction medium is poured into water, the precipitate is filtered, washed with water and dried. 195 mg of desired product are obtained.

### B/ 1-[4-(4-Ethylamino-phenoxy)-2-fluoro-5-methoxy-phenyl]-3-(1-ethyl-propyl)-urea

To a suspension of LAH (63 mg) in THF (3 ml) is added the compound obtained in the preceding step and heated at 60° C. for 10 h. Then a saturated aqueous Na₂SO₄ solution is added, the mixture filtered, extracted with DCM and the organic layer is dried over MgSO₄, filtered and the filtrate evaporated. 44 mg of desired product are isolated after flash chromatography on silica eluting with a DCM/MeOH/NH₄OH mixture (98:2:0.2 v/v/v).

### Preparation 163

### 1-{4-[4-(2-Ethoxy-ethylamino)-phenoxy]-3-methoxy-phenyl}-3-(1-ethyl-propyl)-urea

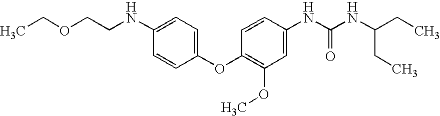

A solution of 1.35 g of compound obtained such as described under Preparation 70, of DIEA (1.36 ml) and of 1-bromo-2-ethoxyethane (0.44 ml) in 80 ml DMF is heated 24 h at 80° C. The reaction medium is concentrated and purified by flash chromatography on silica eluting with a cyclohexane/ethyl acetate mixture (6:4 v/v). 550 mg of desired product are obtained.

### Preparation 164

### 1-(1-Ethyl-propyl)-3-{4-[4-(2-methoxy-ethylamino)-phenoxy]-phenyl}-urea

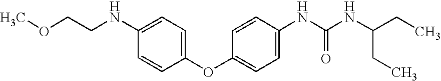

A solution of 1.29 g of compound obtained such as described under Preparation 82, of DIEA (2 ml) and of 2-bromomethylethylether (0.56 ml) in 30 ml DMF is heated 8 h at 80° C. The reaction medium is concentrated, redissolved in DCM, washed with water and the organic layer is dried over MgSO₄, filtered and the filtrate concentrated. 435 mg of desired product are obtained after flash chromatography on silica eluting with a DCM/ethanol/NH₄OH mixture (90:10:0.5 v/v/v).

### Preparation 165

### 1-(1-Ethyl-propyl)-3-{3-methoxy-4-[4-(2-methoxy-ethylamino)-phenoxy]-phenyl}-urea

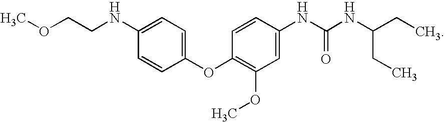

A solution of 2 g of compound obtained such as described under Preparation 70, of DIEA (2.65 ml) and of 2-bromomethylethylether (1.32 ml) in 30 ml DMF is heated 8 h at 80° C. The reaction medium is concentrated, purified by flash chromatography on silica eluting with a cyclohexane/ethyl acetate mixture (6:4 v/v). 734 mg of desired product are obtained.

### Preparation 166

### 1-{3-Ethoxy-4-[4-(2-methoxy-ethylamino)-phenoxy]-phenyl}-3-(1-ethyl-propyl)-urea

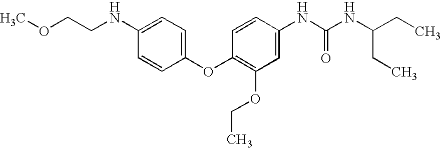

A solution of 2.15 g of compound obtained such as described under Preparation 71, of DIEA (3 ml) and of 2-bromomethylethylether (0.87 ml) in 30 ml DMF is heated 48 h at 80° C. The reaction medium is concentrated, the residue redissolved in ethyl acetate, washed with water and the organic layer is dried over MgSO₄, filtered and the filtrate concentrated to dryness. 766 mg of desired product are obtained after flash chromatography on silica eluting with a cyclohexane/ethyl acetate mixture (55:45 v/v).

### Preparation 167

### 1-(1-Ethyl-propyl)-3-(3-methoxy-4-{4-[(tetrahydro-furan-2-ylmethyl)-amino]-phenoxy}-phenyl)-urea

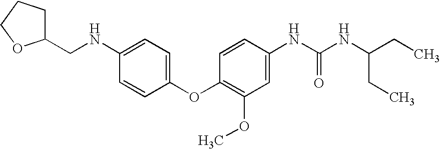

### A/ 2-Bromomethyl-tetrahydro-furane

3.38 ml of PBr₃ are added dropwise to a solution of (tetrahydro-furan-2-yl)-methanol (10.2 g) keeping the temperature to between −5° C. and −10° C. The mixture is stirred 12 h at AT. After diluting with DCM, washing with water, the organic layer is dried over MgSO₄, filtered and the filtrate concentrated. 3 g of desired product are obtained after flash chromatography on silica eluting with a cyclohexane/ethyl acetate mixture (8:2 v/v).

### B/ 1-(1-Ethyl-propyl)-3-(3-methoxy-4-{4-[(tetrahydro-furan-2-ylmethyl)amino]-phenoxy}-phenyl)-urea

A solution of 610 mg of compound obtained such as described under Preparation 70, of DIEA (0.44 ml) and of compound obtained in the preceding step (410 mg) in 6 ml DMF is heated 19 h at 85° C. The reaction medium is poured into water, extracted with DCM and the organic layer is dried over MgSO₄, filtered and the filtrate concentrated. 74 mg of desired product are obtained after flash chromatography on silica eluting with a DCM/MeOH mixture (95:5 v/v).

### Preparation 168

### 1-(1-Ethyl-propyl)-3-{3-methoxy-4-[4-(2-methoxy-propylamino)-phenoxy]-phenyl}-urea

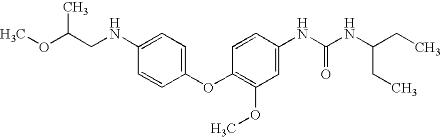

### A/ 2-Methoxy-propionic acid

920 mg of sodium are gradually added to 10 ml of methanol and heated 30 min under reflux, then 2-bromo-propionic acid is added and heating continued for 3 h at 60° C., followed by stirring for 12 h at AT. After concentrating to dryness, the residue is redissolved in water, extracted with TBME, the organic layer is washed with a saturated aqueous NaCl solution and the organic layer dried over MgSO₄, filtered and the filtrate concentrated. 1.5 g of desired product are obtained, which is used as such.

### B/ N-(4-{4-[3-(1-Ethyl-propyl)-ureido]-2-methoxy-phenoxy}-phenyl)-2-methoxy-propionamide

Following General Procedure L1, 180 mg of product of the preceding step are reacted with 500 mg of compound obtained such as described under Preparation 70. The desired product is isolated after chromatography on silica eluting with a DCM/MeOH mixture (95:5 v/v).

### C/ 1-(1-Ethyl-propyl)-3-{3-methoxy-4-[4-(2-methoxy-propylamino)-phenoxy]-phenyl}-urea

A 1M solution of BH₃ in THF (6.18 ml) in 13 ml THF is gradually added to a solution of the compound obtained in the preceding step (564 mg) in 15 ml THF. The mixture is stirred 30 min at AT and heated 15 h under reflux. After return to AT, 5 ml of an aqueous 1N HCl solution is gradually added, the solvent is evaporated, the residue redissolved in water, the medium is basified with a saturated Na₂CO₃ solution, extracted with TBME and the organic layer is dried over MgSO₄, filtered and the filtrate concentrated in vacuo. 150 mg of desired product are obtained after chromatography on silica eluting with a DCM/MeOH mixture (96:4 v/v).

### Preparation 169

### 1-(1-Ethyl-propyl)-3-{3-methoxy-4-[4-tetrahydro-furan-3-ylamino)-phenoxy]-phenyl}-urea

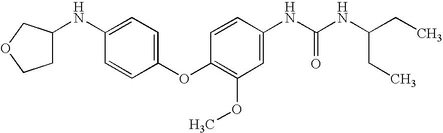

### A/ Methanesulfonate of tetrahydro-furan-3-yl

A solution of 3-hydroxytetrahydrofurane (2.64 g) and TEA (5.05 ml) in DCM (25 ml), is added dropwise under nitrogen to a solution of methanesulfonate chloride (2.67 ml) in DCM (15 ml). After stirring 12 h at AT, the precipitate is filtered, the filtrate washed with water then with an aqueous 1 N HCl solution and with a saturated aqueous solution of NaHCO₃. The organic layer is dried over MgSO₄, filtered and the filtrate concentrated to dryness. 4.4 g of desired product are obtained, which is used as such.

### B/ 1-(1-Ethyl-propyl)-3-{3-methoxy-4-[4-(tetrahydro-furan-3-ylamino)-phenoxy]-phenyl}-urea

A mixture of 687 mg of compound obtained such as described under Preparation 70, of TEA (337 μl) and of the compound obtained in the preceding step (399 mg) in 30 ml of toluene is heated for 3 days under reflux. The reaction medium is concentrated to dryness and purified by flash chromatography on silica eluting with a DCM/MeOH mixture (96:4 v/v). 180 mg of desired product are obtained.

### Preparation 170

### 1-(1-Ethyl-propyl)-3-{3-methoxy-4-[4-(tetrahydro-pyran-4-ylamino)-phenoxy]-phenyl}-urea

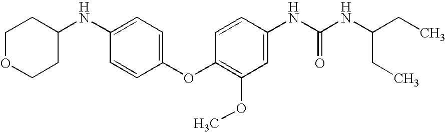

A solution of compound obtained such as described under Preparation 70 (514 mg), of tetrahydro-4H-pyran-4-one (150 mg) in 20 ml DCM and of 10 ml ACN is stirred 2 h at AT. Sodium triacetoxyborohydride (477 mg) is then added gradually and stirred 12 h at AT. 1 ml of saturated aqueous NaHCO₃ solution is added, the organic layer is washed with water, dried over MgSO₄, filtered and concentrated to dryness. The residue is redissolved in diisopropyl ether, and the precipitate is filtered and oven dried. 512 mg of desired product are obtained.

### Preparation 171

### 1-(1-Ethyl-propyl)-3-{3-methoxy-4-[4-(2-methoxy-1-methyl-ethylamino)-phenoxy]-phenyl}-urea

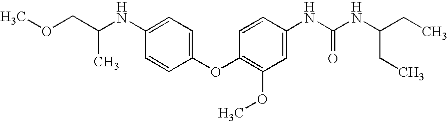

### A/ Methanesulfonate of 2-methoxy-1-methyl-ethyl

Keeping the temperature to below 30° C., a solution of methanesulfonate chloride (7.56 g) in 20 ml DCM is added dropwise to a solution of 1-methoxy-2-propanol (5.4 g) and TEA (10.1 ml) in 50 ml DCM. After stirring 12 h at AT, filtering, the filtrate is washed with water, with an aqueous 1N HCl solution, with a saturated aqueous NaHCO₃ solution and with water. The organic layer is dried over MgSO₄, filtered and concentrated to dryness. 4.4 g of desired product are obtained, which is used as such.

### B/ 1-(1-Ethyl-propyl)-3-{3-methoxy-4-[4-(2-methoxy-1-methyl-ethylamino)-phenoxy]-phenyl}-urea

A solution of 687 mg of compound obtained such as described under Preparation 70, of TEA (309 μl) and of compound obtained in the preceding step (420 mg) in 30 ml of toluene is heated 36 h under reflux. The reaction medium is concentrated to dryness and purified by flash chromatography on silica eluting with DCM/MeOH/NH₄OH mixture (96:4:0.5 v/v/v). 215 mg of desired product are obtained.

### EXAMPLES

### Example 1

### N-(5-{4-[3-(1-Ethyl-propyl)-ureido]-2-methoxymethyl-phenoxy}-thiazol-2-yl)-4-(1-isopropyl-piperidin-4-yloxy)-benzamide

### A/ 4-(1-Isopropyl-piperidin-4-yloxy)-benzotriazol-1-yl benzoate

A mixture of 1.053 g of 4-(1-Isopropyl-piperidin-4-yloxy)-benzoic acid, 0.835 g TBTU, 0.351 g HOBT and 1.04 mL of DIEA in 9.5 mL DCM is stirred at AT for 1 h, then diluted with 200 mL DCM, the organic layer is washed with a dilute aqueous NaOH solution, with a dilute aqueous HCl solution, and the organic layer is dried over magnesium sulfate, filtered and the solvent evaporated in vacuo at 60° C. The desired product is obtained, which is used as such.

### B/ N-(5-{4-[3-(1-Ethyl-propyl)-ureido]-2-methoxymethyl-phenoxy}-thiazol-2-yl)-4-(1-isopropyl-piperidin-4-yloxy)-benzamide

0.130 g of compound of the preceding step and 0.110 g of 1-[4-(2-Amino-thiazol-5-yloxy)-3-methoxymethyl-phenyl]-3-(1-ethyl-propyl)-urea are placed in solution in 8 mL DMF at AT, the solvent is evaporated in vacuo at 60° C. and the mixture held in vacuo at 60° C. for 3 h and then at AT for 15 h. The reaction mixture is purified by semi-preparative HPLC. The solvent of the HPLC fractions is evaporated in vacuo, the residue is precipitated with ethyl ether, filtered and the powder dried. The desired product is thus obtained in TFA salt form.

Following the same operating mode as described in Example 1, the following compounds are obtained:

### N-(5-{4-[3-(1-Ethyl-propyl)-ureido]-2-methoxy-phenoxy}-thiazol-2-yl)-4-(1-isopropyl-piperidin-4-yloxy)-benzamide

### Example 2

The desired product is obtained by reaction of 4-(1-Isopropyl-piperidin-4-yloxy)-benzotriazol-1-yl benzoate and of 1-[4-(2-Amino-thiazol-5-yloxy)-3-methoxy-phenyl]-3-(1-ethyl-propyl)-urea.

MS (APCI⁺): 596 (M+H)⁺

Elemental analysis: found C, 54.49. H, 6.09. N, 9.60. calculated for C₃₅H₄₄N₄O₅.1C₂HF₃O₂.1H₂O C, 54.46. H, 6.09. N, 9.62

### N-(5-{4-[3-(1-Ethyl-propyl)-ureido]-2-methylcarbamoylmethyl-phenoxy}-thiazol-2-yl)-4-(1-isopropyl-piperidin-4-yloxy)-benzamide

### Example 3

The desired product is obtained by reaction of 4-(1-Isopropyl-piperidin-4-yloxy)-benzotriazol-1-yl benzoate with 2-{2-(2-Amino-thiazol-5-yloxy)-5-[3-(1-ethyl-propyl)-ureido]-phenyl}-N-methyl-acetamide.

### N-{5-[4-(3-dimethylamino-ureido)-2-methylcarbamoylmethyl-phenoxy]-thiazol-2-yl}-4-(1-isopropyl-piperidin-4-yloxy)-benzamide

### Example 4

The desired product is obtained by reaction of 4-(1-Isopropyl-piperidin-4-yloxy)-benzotriazol-1-yl benzoate with 2-[2-(2-Amino-thiazol-5-yloxy)-5-(3-dimethylamino-ureido)-phenyl]-N-methyl-acetamide.

### N-(4-{4-[3-(1-Ethyl-propyl)-ureido]-2-methoxymethyl-phenoxy}-phenyl)-4-(1-isopropyl-piperidin-4-yloxy)-benzamide

### Example 5

The desired product is obtained by reaction of 4-(1-Isopropyl-piperidin-4-yloxy)-benzotriazol-1-yl benzoate with 1-[4-(4-Amino-phenoxy)-3-methoxymethyl-phenyl]-3-(1-ethyl-propyl)-urea in the presence of 1 eq of DIEA.

### N-(4-{4-[3-(1-Ethyl-propyl)-ureido]-2-methoxy-phenoxy}-3-methyl-phenyl)-4-(1-isopropyl-piperidin-4-yloxy)-benzamide

### Example 6

The desired product is obtained by reaction of 4-(1-Isopropyl-piperidin-4-yloxy)-benzotriazol-1-yl benzoate with 1-[4-(4-Amino-2-methyl-phenoxy)-3-methoxy-phenyl]-3-(1-ethyl-propyl)-urea in the presence of 1 eq of DIEA.

### Example 7

### N-5-{4-[3-(1-Ethyl-propyl)-ureido]-2-methoxymethyl-phenoxy}-thiazol-2-yl)-4-(1-isopropyl-piperidin-4-yloxy)-N-methyl-benzamide

0.265 g of 4-(1-Isopropyl-piperidin-4-yloxy)-benzotriazol-1-yl benzoate and 0.240 g of 1-(1-Ethyl-propyl)-3-[3-methoxymethyl-4-(2-methylamino-thiazol-5-yloxy)-phenyl]-urea are placed in solution in 1.5 mL of DMF, the mixture is held at AT for 48 h, the solvent evaporated in vacuo, the residue is redissolved in water, extracted with ethyl acetate and the organic layer is dried over MgSO₄, filtered and evaporated in vacuo. The residue thus obtained is purified by chromatography on silica eluting with a DCM/MeOH/NH₄OH mixture (95:5:0.5 v/v/v). The desired product is isolated in the form of a free base.

### Example 8

### N-(4-{4-[3-(1-Ethyl-propyl)-ureido]-phenoxy}-3-methyl-phenyl)-4-(1-isopropyl-piperidin-4-yloxy)-benzamide

0.233 g of 4-(1-Isopropyl-piperidin-4-yloxy)-benzotriazol-1-yl benzoate and 0.145 g of 1-[4-(4-Amino-2-methyl-phenoxy)-phenyl]-3-(1-ethyl-propyl)-urea are placed in solution in 3 mL DMF, the mixture is held at AT for 48 h and the solvent is evaporated in vacuo. The residue obtained is purified by chromatography on silica eluting with the mixtures AcOEt/MeOH/NH₄OH (9:1:0.5 v/v/v), DCM/MeOH/NH₄OH (8:2:0.5 v/v/v) and finally by semi-preparative HPLC. The product is isolated in TFA salt form following the operating mode described in Example 1.

### Example 9

### N-(5-{4-[3-(1-Ethyl-propyl)-ureido]-2-methoxymethyl-phenoxy}-thiazol-2-yl)-4-(1-isopropyl-piperidin-3-ylmethoxy)-benzamide

### A/ 4-(1-Isopropyl-piperidin-3-ylmethoxy)-benzotriazol-1-yl benzoate

A mixture of 0.166 g of 4-(1-isopropyl-piperidin-3-ylmethoxy)-benzoic acid, 89 mg of HOBT, 211 mg TBTU and 0.335 ml DIEA in 15 ml DCM is stirred at AT for 1 h. The desired product is isolated following the operating mode described in Example 1, step A.

### B/ N-(5-{4-[3-(1-Ethyl-propyl)-ureido]-2-methoxymethyl-phenoxy}-thiazol-2-yl)-4-(1-isopropyl-piperidin-3-ylmethoxy)-benzamide

The compound of the preceding step is reacted with 122 mg of amine 1-[4-(2-amino-thiazol-5-yloxy)-3-methoxymethyl-phenyl]-3-(1-ethyl-propyl)-urea in DMF, for 6 h at AT. After evaporation in vacuo the residue is purified by semi-preparative HPLC. The desired product is isolated in TFA salt form, following the operating mode described in Example 1.

### Example 10

### 4-(1-Butyl-piperidin-4-yloxy)-N-(5-{4-[3-(1-ethyl-propyl)-ureido]-2-methoxy-phenoxy}-thiazol-2-yl)-benzamide

### A/ 4-(1-Butyl-piperidin-4-yloxy)-benzotriazol-1-yl benzoate

A mixture of 0.832 g of 4-(1-Butyl-piperidin-4-yloxy)-benzoic acid, 1.060 g of TBTU, 0.440 g HOBT and 1.68 mL DIEA in 30 mL DCM is stirred at AT for 1 h. The desired product is isolated following the operating mode described in Example 1, step A.

### B/ 4-(1-Butyl-piperidin-4-yloxy)-N-(5-{4-[3-(1-ethyl-propyl)-ureido]-2-methoxy-phenoxy}-thiazol-2-yl)-benzamide

The compound of the preceding step and 0.235 g of 1-[4-(2-Amino-thiazol-5-yloxy)-3-methoxy-phenyl]-3-(1-ethyl-propyl)-urea are placed in solution in 10 mL DMF at AT, the solvent is evaporated in vacuo and the mixture is held under a vacuum at 60° C. for 5 h and then for 15 h at AT. The desired product is isolated in TFA salt form, after semi-preparative HPLC purification, following the operating mode described in Example 1.

Following the same operating mode as described in Example 10, the following compounds are obtained:

### 4-(1-Butyl-piperidin-4-yloxy)-N-(5-{4-[3-(1-ethyl-propyl)-ureido]-2-methoxymethyl-phenoxy}-thiazol-2-yl)-benzamide

### Example 11

The desired product is obtained by reaction of 4-(1-Butyl-piperidin-4-yloxy)-benzotriazol-1-yl benzoate with 1-[4-(2-Amino-thiazol-5-yloxy)-3-methoxymethyl-phenyl]-3-(1-ethyl-propyl)-urea.

### 4-(1-Butyl-piperidin-4-yloxy)-N-(5-{4-[3-(1-ethyl-propyl)-ureido]-2-fluoro-phenoxy}-thiazol-2-yl)-benzamide

### Example 12

The desired product is obtained by reaction of 4-(1-Butyl-piperidin-4-yloxy)-benzotriazol-1-yl benzoate with 1-[4-(2-Amino-thiazol-5-yloxy)-3-fluoro-phenyl]-3-(1-ethyl-propyl)-urea.

### 4-(1-Butyl-piperidin-4-yloxy)-N-(5-{2-ethoxymethyl-4-[3-(1-ethyl-propyl)-ureido]-phenoxy}-thiazol-2-yl)-benzamide

### Example 13

The desired product is obtained by reaction of 4-(1-Butyl-piperidin-4-yloxy)-benzotriazol-1-yl benzoate with 1-[4-(2-Amino-thiazol-5-yloxy)-3-ethoxymethyl-phenyl]-3-(1-ethyl-propyl)-urea.

### 4-(1-Butyl-piperidin-4-yloxy)-N-(4-{2-ethoxy-4-[3-(1-ethyl-propyl)-ureido]-phenoxy}-phenyl)-benzamide

### Example 14

The desired product is obtained by reaction of 4-(1-Butyl-piperidin-4-yloxy)-benzotriazol-1-yl benzoate with 1-[4-(4-Amino-phenoxy)-3-ethoxy-phenyl]-3-(1-ethyl-propyl)-urea

### 4-(1-Butyl-piperidin-4-yloxy)-N-(4-{2-chloro-4-[3-(1-ethyl-propyl)-ureido]-phenoxy}-phenyl)-benzamide

### Example 15

The desired product is obtained by reaction of 4-(1-Butyl-piperidin-4-yloxy)-benzotriazol-1-yl benzoate with 1-[4-(4-Amino-phenoxy)-3-chloro-phenyl]-3-(1-ethyl-propyl)-urea.

### 4-(1-Butyl-piperidin-4-yloxy)-N-{3-[4-(3-dimethylamino-ureido)-2-methoxy-phenoxy]-phenyl}-benzamide

### Example 16

The desired product is obtained by reaction of 4-(1-Butyl-piperidin-4-yloxy)-benzotriazol-1-yl benzoate with 1-[4-(3-Amino-phenoxy)-3-methoxy-phenyl]-3-dimethylamino-urea.

### Example 17

### 4-(1-Butyl-piperidin-4-yloxy)-N-(4-{4-[3-(1-ethyl-propyl)-ureido]-2-meth phenoxy}-phenyl)-N-(2-hydroxy-ethyl)-benzamide

0.300 g of 1-(1-Ethyl-propyl)-3-{4-[4-(2-hydroxy-ethylamino)-phenoxy]-3-methoxy-phenyl}-urea and 4-(1-Butyl-piperidin-4-yloxy)-benzotriazol-1-yl benzoate (1 eq) are solubilized in 5 mL DMF at AT, the solvent is evaporated in vacuo at 60° C. and the residue held in vacuo 1 h at 60° C. The desired product is isolated in the form of a free base after chromatography on silica eluting with a DCM/MeOH/NH₄OH mixture (90:10:0.1 v/v/v). The hydrochloride is obtained by treating the base with a HCl/diethyl ether mixture.

### Example 18

### 4-(1-Butyl-piperidin-4-yloxy)-N-(4-{4-[3-(1-ethoxy-propyl)-ureido]-2-methoxy-phenoxy}-phenyl)-N-3,3,3-trifluoro-propyl)-benzamide

0.630 g of 4-(1-Butyl-piperidin-4-yloxy)-benzotriazol-1-yl benzoate and 0.350 g of 1-(1-Ethyl-propyl)-3-{3-methoxy-4-[4-(3,3,3-trifluoro-propylamino)-phenoxy]-phenyl}-urea are placed in solution in 5 mL of DMF at AT, the solvent is evaporated in vacuo at 60° C. and the residue is held in a vacuum at 60° C. for 15 h. The desired product is isolated in the form of a free base after chromatography on silica eluting with a cyclohexane/ethyl acetate/TEA mixture (60:40:30 v/v/v). The hydrochloride is obtained by treating the base with a HCl/diethyl ether mixture.

### Example 19

### 4-(1-Butyl-piperidin-4-yloxy)-N-(4-{4-[3-(1-ethyl-propyl)-ureido]-2-methoxy-phenoxy}-phenyl)-N-(3-methoxy-propyl)-benzamide

0.125 g of 1-(1-Ethyl-propyl)-3-{3-methoxy-4-[4-(3-methoxy-propylamino)-phenoxy]-phenyl}-urea and 4-(1-Butyl-piperidin-4-yloxy)-benzotriazol-1-yl benzoate (1 eq) are placed in solution in 5 mL DMF at AT, the solvent is evaporated in vacuo at 60° C. and the residue held in vacuo at 60° C. for 3 h. The residue is purified by semi-preparative HPLC. The solvent of the HPLC fractions is evaporated, the residue is redissolved in DCM, the organic layer is washed with an aqueous Na₂CO₃ solution and dried over MgSO₄, filtered, a HCl/diethyl ether mixture is added, and the solvent is evaporated in vacuo. The powder obtained is washed with diethyl ether. The desired product is obtained in hydrochloride form.

### Example 20

### 4-(1-Butyl-piperidin-4-yloxy)-N-(4-{4-[3-(1-ethyl-propyl)-ureido]-2-methoxy-phenoxy}-phenyl)-N-(4,4,4-trifluoro-butyl)-benzamide

The desired product is obtained by reaction of 4-(1-Butyl-piperidin-4-yloxy)-benzotriazol-1-yl benzoate with 1-(1-Ethyl-propyl)-3-{3-methoxy-4-[4-(4,4,4-trifluoro-butylamino)-phenoxy]-phenyl}-urea, following the operating mode described in Example 19.

### Example 21

### 4-(1-Butyl-piperidin-4-yloxy)-N-(4-{4-[3-(1-ethyl-propyl)-ureido]-2-isopropyl-phenoxy}-phenyl)-benzamide

0.420 g of 1-[4-(4-Amino-phenoxy)-3-isopropyl-phenyl]-3-(1-ethyl-propyl)-urea and 4-(1-Butyl-piperidin-4-yloxy)-benzotriazol-1-yl benzoate (1 eq) are placed in solution in 10 mL DMF at AT, the solvent is evaporated in vacuo at 60° C. and the residue held in vacuo at 60° C. for 5 h and at AT for 48 h. The residue is redissolved in ACN; the precipitate obtained is filtered, solubilized in DCM, and the organic layer is washed with an aqueous NaOH solution, dried over MgSO₄, filtered and evaporated. The desired product is obtained in the form of a free base which is converted into a hydrochloride in the presence of a HCl/diethyl ether mixture.

### Example 22

### 4-(1-Butyl-piperidin-4-yloxy)-N-(4{-2-ethoxy-4-[3-(1-ethyl-propyl)-ureido]-phenoxy}-2-fluoro-phenyl)-3-methyl-benzamide

### A/ 4-(1-Butyl-piperidin-4-yloxy)-3-methyl-benzotriazol-1-yl benzoate

A mixture of 0.520 g of 4-(1-Butyl-piperidin-4-yloxy)-3-methyl-benzoic acid, 0.565 g of TBTU, 0.240 g of HOBT and 1.20 mL of DIEA in 10 mL DCM is stirred at AT for 1 h. The desired product is isolated following the operating mode described in Example 1, step A.

### B/ 4-(1-Butyl-piperidin-4-yloxy)-N-(4-{2-ethoxy-4-[3-(1-ethyl-propyl)-ureido]-phenoxy}-2-fluorophenyl)-3-methyl-benzamide

The compound of the preceding step and 0.300 g of 1-[4-(4-Amino-3-fluoro-phenoxy)-3-ethoxy-phenyl]-3-(1-ethyl-propyl)-urea are placed in solution in 20 mL of a DMF/DCM mixture (1:1 v/v) at AT, the solvent is evaporated in vacuo and the residue obtained is held in vacuo at 60° C. for 15 h. The desired product is isolated in the form of a free base after chromatography on silica eluting with a DCM/MeOH/NH₄OH mixture (98:2:0.2). The hydrochloride is obtained by treating the base with a HCl/diethyl ether mixture.

### Example 23

### 4-(1-Butyl-piperidin-4-yloxy)-N-(4-{4-[3-(1-ethyl-propyl)-ureido]-2-methoxy-phenoxy}-phenyl)-3-methyl-benzamide

0.172 g of 1-[4-(4-Amino-phenoxy)-3-methoxy-phenyl]-3-(1-ethyl-propyl)-urea and 4-(1-Butyl-piperidin-4-yloxy)-benzotriazol-1-yl benzoate (1 eq) are placed in solution in 10 mL DMF at AT, the solvent is evaporated in vacuo at 60° C. and the residue held in vacuo at 60° C. for 1 h and 15 h at AT. The residue is purified by semi-preparative HPLC. The desired product is isolated in hydrochloride form following the operating mode described in Example 19.

### Example 24

### 4-(1-Butyl-piperidin-4-yloxy)-N-(4-{4-[3-(1-ethyl-propyl)-ureido]-2-methoxy-phenoxy}-phenyl)-3-methoxy-benzamide

### A/ 4-(1-Butyl-piperidin-4-yloxy)-3-methoxy-benzotriazol-1-yl benzoate

A mixture of 0.307 g of 4-(1-Butyl-piperidin-4-yloxy)-3-methoxy-benzoic acid, 0.418 g of TBTU, 0.176 g of HOBT and 0.52 mL of DIEA in 20 mL DCM is stirred at AT for 1 h. The desired product is isolated following the operating mode described in Example 1, step A.

### B/ 4-(1-Butyl-piperidin-4-yloxy)-N-(4-{4-[3-(1-ethyl-propyl)-ureido]-2-methoxy-phenoxy}-phenyl)-3-methoxy-benzamide

The compound of the preceding step and 0.202 g of 1-[4-(4-Amino-phenoxy)-3-methoxy-phenyl]-3-(1-ethyl-propyl)-urea are placed in solution in 10 mL DMF at AT, evaporated in vacuo and the residue obtained is held in vacuo at 60° C. for 1 h and 15 h at AT. The residue is purified by semi-preparative HPLC. The desired product is isolated in TFA salt form following the operating mode described in Example 1.

### Example 25

### 4-(1-Butyl-piperidin-4-yloxy)-3-chloro-N-(4-{4-[3-(1-ethyl-propyl)-ureido]-2-methoxy-phenoxy}-phenyl)-benzamide

### A/ 4-(1-Butyl-piperidin-4-yloxy)-3-chloro-benzotriazol-1-yl benzoate

A mixture of 0.312 g of 4-(1-Butyl-piperidin-4-yloxy)-3-chloro-benzoic acid, 0.418 g of TBTU, 0.176 g HOBT and 0.52 mL DIEA in 20 mL DCM is stirred at AT for 1 h. The desired product is isolated following the operating mode described in Example 1, step A.

### B/ 4-(1-Butyl-piperidin-4-yloxy)-3-chloro-N-(4-{4-[3-(1-ethyl-propyl)-ureido]-2-methoxy-phenoxy}-phenyl)-benzamide

Following the operating mode described in Example 24 the desired product is obtained from the compound of the preceding step and 0.202 g of 1-[4-(4-Amino-phenoxy)-3-methoxy-phenyl]-3-(1-ethyl-propyl)-urea.

### Example 26

### N-(5-{4-[3-(1-Ethyl-propyl)-ureido]-2-methoxy-phenoxy}-thiazol-2-yl)-4-(1-methyl-piperidin-4-yloxy)-benzamide

### A/ 4-(1-Methyl-piperidin-4-yloxy)-benzotriazol-1-yl benzoate

A mixture of 0.500 g of 4-(1-Methyl-piperidin-4-yloxy)-benzoic acid, 0.768 g of TBTU, 0.323 g HOBT and 1.27 mL of DIEA in 30 mL DCM is stirred at AT for 1 h. The desired product is isolated following the operating mode described in Example 1, step A.

### B/ N-(5-{4-[3-(1-Ethyl-propyl)-ureido]-2-methoxy-phenoxy}-thiazol-2-yl)-4-(1-methyl-piperidin-4-yloxy)-benzamide

The desired product is obtained from the compound of the preceding step and 0.140 g of 1-[4-(2-Amino-thiazol-5-yloxy)-3-methoxy-phenyl]-3-(1-ethyl-propyl)-urea, following the operating mode described in Example 24.

### Example 27

### N-(5-{4-[3-(1-Ethyl-propyl)-ureido]-2-methoxymethyl-phenoxy}-thiazol-2-yl)-4-(1-isopropyl-pyrrolidin-3-yloxy)-benzamide

### A/ 4-(1-Isopropyl-pyrrolidin-3-yloxy)-benzotriazol-1-yl benzoate

A mixture of 0.800 g of 4-(1-Isopropyl-pyrrolidin-3-yloxy)-benzoic acid, 1.120 g of TBTU, 0.480 HOBT and 1.80 mL DIEA in 30 mL DCM is stirred at AT for 1.5 h. The desired product is isolated following the operating mode described in Example 1, step A.

### B/ N-(5-{4-[3-(1-Ethyl-propyl)-ureido]-2-methoxymethyl-phenoxy}-thiazol-2-yl)-4-(1-isopropyl-pyrrolidin-3-yloxy)-benzamide

0.200 g of 1-[4-(2-Amino-thiazol-5-yloxy)-3-methoxymethyl-phenyl]-3-(1-ethyl-propyl)-urea and the compound of the preceding step (1 eq) are placed in solution in 2 ml DMF at AT, evaporated in vacuo at 60° C., holding the mixture at 60° C. for 4 h and at AT for 48 h. The desired product is isolated in the form of a free base after chromatography on silica eluting with a DCM/MeOH/NH₄OH mixture (95:5:0.5 v/v/v), followed by precipitation and washing with isopropyl alcohol and with pentane.

### Example 28

### N-(5-{4-[3-(1-Ethyl-propyl)-ureido]-2-methoxymethyl-phenoxy}-thiazol-2-yl)-3-(1-isopropyl-piperidin-4-yloxy)-benzamide

### A/ 3-(1-Isopropyl-piperidin-4-yloxy)-benzotriazol-1-yl benzoate

A mixture of 0.130 g of 3-(1-Isopropyl-piperidin-4-yloxy)-benzoic acid, 0.175 g of TBTU, 0.175 g HOBT and 0.28 mL of DIEA in 20 mL DCM is stirred at AT for 0.5 h. The desired product is isolated following the operating mode described in Example 1, step A.

### B/ N-(5-{4-[3-(1-Ethyl-propyl)-ureido]-2-methoxymethyl-phenoxy}-thiazol-2-yl)-3-(1-isopropyl-piperidin-4-yloxy)-benzamide

The desired product is obtained from the compound of the preceding step and from 0.124 g of 1-[4-(2-Amino-thiazol-5-yloxy)-3-methoxymethyl-phenyl]-3-(1-ethyl-propyl)-urea, following the operating mode described in Example 24.

Following the same operating mode as described for Example 28, the following compounds are obtained:

### N-(4-{3-[3-(1-Ethyl-propyl)-ureido]-phenoxy}-3-methyl-phenyl)-3-(1-isopropyl-piperidin-4-yloxy)-benzamide

### Example 29

The desired product is obtained by reaction of 3-(1-isopropyl-piperidin-4-yloxy)-benzotriazol-1-yl benzoate with 1-[4-(4-Amino-2-methyl-phenoxy)-phenyl]-3-(1-ethyl-propyl)-urea in the presence of 1 eq of DIEA.

### N-(4-{3-[3-(1-Ethyl-propyl)-ureido]-phenoxy}-3-methoxymethyl-phenyl)-3-(1-isopropyl-piperidin-4-yloxy)-benzamide

### Example 30

The desired product is obtained by reaction of 3-(1-isopropyl-piperidin-4-yloxy)-benzotriazol-1-yl benzoate with 1-[4-(4-Amino-phenoxy)-3-methoxymethyl-phenyl]-3-(1-ethyl-propyl)-urea.

### N-(4-{5-[3-(1-Ethyl-propyl)-ureido]-2-methoxy-phenoxy}-phenyl)-3-(1-isopropyl-piperidin-4-yloxy)-benzamide

### Example 31

The desired product is obtained by reaction of 3-(1-isopropyl-piperidin-4-yloxy)-benzotriazol-1-yl benzoate with 1-[4-(4-Amino-phenoxy)-3-methoxy-phenyl]-3-(1-ethyl-propyl)-urea in the presence of 1 eq of DIEA.

### Example 32

### 4-(1-Benzyl-piperidin-4-yloxy)-N-(5-{4-[3-(1-ethyl-propyl)-ureido]-2-methoxy-phenoxy}-thiazol-2-yl)-benzamide

### A/ 4-(1-Benzyl-piperidin-4-yloxy)-benzotriazol-1-yl benzoate

A mixture of 1 g of 4 (1-Benzyl-piperidin-4-yloxy)-benzoic acid, 1.09 g of TBTU, 0.457 g of HOBT and 2.25 mL of DIEA in 200 mL DCM is stirred at AT for 1 h. The desired product is isolated following the operating mode described in Example 1, step A.

### B/ 4-Benzyl-piperidin-4-yloxy N-(5-{4-[3-(1-ethyl-propyl)-ureido]-2-methoxy-phenoxy}-thiazol-2-yl)-benzamide

The desired product is obtained from 0.200 g of 1-[4-(2-Amino-thiazol-5-yloxy)-3-methoxy-phenyl]-3-(1-ethyl-propyl)-urea and the compound of the preceding step (1 eq), following the operating mode described in Example 24.

### Example 33

### N-(5-{4-[3-(1-Ethyl-propyl)-ureido]-2-methoxymethyl-phenoxy}-thiazol-2-yl)-4-(1-isopropyl-piperidin-3-yloxy)-benzamide

### A/ 4-(1-Isopropyl-piperidin-3-yloxy)-benzotriazol-1-yl benzoate

A mixture of 0.260 g of 4-(1-Isopropyl-piperidin-3-yloxy)-benzoic acid, 0.350 g of TBTU, 0.150 g HOBT and 0.56 mL of DIEA in 30 mL DCM is stirred at AT for 0.5 h. The desired product is isolated following the operating mode described in Example 1, step A.

### B/ N-(5-{4-[3-(1-Ethyl-propyl)-ureido]-2-methoxymethyl-phenoxy}-thiazol-2-yl)-4-(1-isopropyl-piperidin-3-yloxy)-benzamide

The desired product is obtained from 0.360 g of 1-[4-(2-Amino-thiazol-5-yloxy)-3-methoxymethyl-phenyl]-3-(1-ethyl-propyl)-urea and the compound obtained in the preceding step (1 eq), following the operating mode described in Example 24, the heating step at 60° C. being conducted for 4 h.

Following the same operating mode as described in Example 33, the following compounds are obtained:

### N-(4-{3-[3-(1-Ethyl-propyl)-ureido]-phenoxy}-3-methyl-phenyl)-4-(1-isopropyl-piperidin-3-yloxy)-benzamide

### Example 34

The desired product is obtained by reaction of 4-(1-Isopropyl-piperidin-3-yloxy)-benzotriazol-1-yl benzoate with 1-[4-(4-Amino-2-methyl-phenoxy)-phenyl]-3-(1-ethyl-propyl)-urea in the presence of 1 eq of DIEA.

### N-(4-{5-[3-(1-Ethyl-propyl)-ureido]-2-methoxy-phenoxy}-phenyl)-4-(1-isopropyl-piperidin-3-yloxy)-benzamide

### Example 35

The desired product is obtained by reaction of 4-(1-Isopropyl-piperidin-3-yloxy)-benzotriazol-1-yl benzoate with 1-[4-(4-Amino-phenoxy)-3-methoxy-phenyl]-3-(1-ethyl-propyl)-urea in the presence of 1 eq of DIEA.

### N-{3-[4-(3-dimethylamino-ureido)-2-methoxy-phenoxy]-phenyl}-4-(1-isopropyl-piperidin-3-yloxy)-benzamide

### Example 36

### The desired product is obtained by reaction of 4-(1-Isopropyl-piperidin-3-yloxy)-benzotriazol-1-yl benzoate with 1-[4-(3-Aminophenoxy)-3-methoxy-phenyl]-3-dimethylamino-urea in the presence of 1 eq of DIEA.

### Example 37

### N-(5-{4-[3-(1-Ethyl-propyl)-ureido]-2-methoxy-phenoxy}-thiazol-2-yl)-4-(1-isopropyl-piperidin-4-yloxymethyl)-benzamide

### A/ 4-(1-Isopropyl-piperidin-4-yloxymethyl)-benzotriazol-1-yl benzoate

A mixture of 0.150 g of 4-(1-Isopropyl-piperidin-4-yloxymethyl)-benzoic acid, 0.190 g TBTU, 0.081 g HOBT and 0.30 mL of DIEA in 5 mL DCM is stirred at AT for 1 h. The desired product is isolated following the operating mode described in Example 1, step A.

### B/ N-(5-{4-[3-(1-Ethyl-propyl)-ureido]-2-methoxy-phenoxy}-thiazol-2-yl)-4-(1-isopropyl-piperidin-4-yloxymethyl)-benzamide

0.131 g of 1-[4-(2-Amino-thiazol-5-yloxy)-3-methoxy-phenyl]-3-(1-ethyl-propyl)-urea and the compound of the preceding step are placed in solution in 1 mL DMF, heated at 70° C. for 24 h and evaporated in vacuo. The residue is purified by semi-preparative HPLC. The desired product is isolated in TFA salt form, following the operating mode described in Example 1.

### Example 38

### N-(5-{4-[3-(1-Ethyl-propyl)-ureido]-2-methoxymethyl-phenoxy}-thiazol-2-yl)-3-(1-isopropyl-piperidin-3-yloxy)-benzamide

### A/ 3-(1-Isopropyl-piperidin-3-yloxy)-benzotriazol-1-yl benzoate

A mixture of 0.260 g of 3-(1-Isopropyl-piperidin-3-yloxy)-benzoic acid, 0.350 g of TBTU, 0.150 g HOBT and 0.56 mL of DIEA in 30 mL DCM is stirred at AT for 1 h. The desired product is isolated following the operating mode described in Example 1, step A.

### B/ N-(5-{4-[3-(1-Ethyl-propyl)-ureido]-2-methoxymethyl-phenoxy}-thiazol-2-yl)-3-(1-isopropyl-piperidin-3-yloxy)-benzamide

The desired product is obtained from 0.290 g of 1-[4-(2-Amino-thiazol-5-yloxy)-3-methoxymethyl-phenyl]-3-(1-ethyl-propyl)-urea and the compound of the preceding step, following the operating mode described in Example 33.

### Example 39

### N-(5-{4-[3-(1-Ethyl-propyl)-ureido]-2-methoxy-phenoxy}-thiazol-2-yl)-4-(1-isopropyl-piperidin-4-ylmethoxy)-benzamide

### A/ 4-(1-Isopropyl-piperidin-4-ylmethoxy)-benzotriazol-1-yl benzoate

A mixture of 0.270 g of 4-(1-Isopropyl-piperidin-4-ylmethoxy)-benzoic acid, 0.302 g of TBTU, 0.130 g HOBT and 0.63 mL DIEA in 8 mL DCM is stirred at AT for 1 h. The desired product is isolated following the operating mode described in Example 1, step A.

### B/ N-(5-{4-[3-(1-Ethyl-propyl)-ureido]-2-methoxy-phenoxy}-thiazol-2-yl)-4-(1-isopropyl-piperidin-4-ylmethoxy)-benzamide

The compound of the preceding step and 0.150 g of 1-[4-(2-Amino-thiazol-5-yloxy)-3-methoxy-phenyl]-3-(1-ethyl-propyl)-urea are placed in solution in 1 mL DMF, the solvent is evaporated in vacuo at 60° C. and the mixture is held in vacuo at 60° C. for 5 h and 48 h at AT. The reaction medium is redissolved in water, extracted with DCM, and the organic layer is dried over MgSO₄, filtered and evaporated. The desired product is isolated in the form of a free base after chromatography on silica eluting with a DCM/MeOH/NH₄OH mixture (95:5:0.5 v/v/v).

### Example 40

### N-(4-{2-Ethoxy-4-[3-(1-ethyl-propyl)-ureido]-phenoxy}-phenyl)-4-(8-methyl-8-aza-bicyclo[3.2.1]oct-(3-endo)-yloxy)-benzamide

### A/ 4-(8-Methyl-8-aza-bicyclo[3.2.1]oct-(3-endo)-yloxy)-benzotriazol-1-yl benzoate

A mixture of 1.050 g of 4-(8-Methyl-8-aza-bicyclo[3.2.1]oct-(3-endo)-yloxy)-benzoic acid, 1.670 g of TBTU, 0.700 g HOBT and 2.08 mL of DIEA in 20 mL DCM is stirred at AT for 1 h. The desired product is isolated following the operating mode described in Example 1, step A.

### B/ N-(4-{2-Ethoxy-4-[3-(1-ethyl-propyl)-ureido]-phenoxy}-phenyl)-4-(8-methyl-8-aza-bicyclo[3.2.1]oct-(3-endo)-yloxy)-benzamide

The compound of the preceding step and 0.358 g of 1-[4-(4-Amino-phenoxy)-3-ethoxy-phenyl]-3-(1-ethyl-propyl)-urea are placed in solution in 4 mL DMF, evaporated in vacuo at 60° C. and the mixture held in vacuo at 60° C. for 5 h and 15 h at AT. The residue is purified by semi-preparative HPLC. The desired product is isolated in hydrochloride form following the operating mode described in Example 19.

¹H NMR: 10.25 (s, 1H); 10.03 (s, 1H); 8.52 (s, 1H); 7.97-7.95 (d, 2H); 7.65-7.63 (d, 2H); 7.39 (s; 1H); 7.09-7.07 (d, 2H); 6.90-6.88 (d, 1H); 6.83-6.78 (m, 3H); 6.00 (d, 1H); 4.82 (m, 1H); 3.98-3.86 (m; 4H); 3.45 (m, 1H); 2.68-2.67 (d, 3H); 2.50 (m, 2H); 2.23-2.10 (m, 6H); 1.50-1.40 (m, 2H); 1.40-1.30 (m, 2H); 1.19-1.15 (t, 3H); 0.87-0.84 (t, 6H)

MS (APCI⁺): 601 (M+H)⁺

Elemental analysis: found C, 64.13. H, 7.16. N, 8.55. calculated for C₃₅H₄₄N₄O₅.1HCl.1H₂O C, 64.16. H, 7.23. N, 8.55

Following the same operating mode as described in Example 40, the following compounds are obtained:

### N-(4-{4-[3-(1-Ethyl-propyl)-ureido]-2-methoxy-phenoxy}-phenyl)-4-(8-methyl-8-aza-bicyclo[3.2.1]oct-(3-endo)-yloxy)-benzamide

### Example 41

The desired product is obtained by reaction of 4-(8-Methyl-8-aza-bicyclo[3.2.1]oct-3-endo)-yloxy)-benzotriazol-1-yl benzoate with 1-[4-(4-Amino-phenoxy)-3-methoxy-phenyl]-3-(1-ethyl-propyl)-urea.

### N-(4-{2-Ethoxy-4-[3-(1-ethyl-propyl)-ureido]-phenoxy}-phenyl)-N-ethyl-4-(8-methyl-8-aza-bicyclo[3.2.1]oct-(3-endo)-yloxy)-benzamide

### Example 42

The desired product is obtained by reaction of 4-(8-Methyl-8-aza-bicyclo[3.2.1]oct-3-endo)-yloxy)-benzotriazol-1-yl benzoate with 1-[3-Ethoxy-4-(4-ethylamino-phenoxy)-phenyl]-3-(1-ethyl-propyl)-urea.

### N-Ethyl-N-(4-{4-[3-(1-ethyl-propyl)-ureido]-2-methoxy-phenoxy}-phenyl)-4-(8-methyl-8-aza-bicyclo[3.2.1]oct-(3-endo)-yloxy)-benzamide

### Example 43

The desired product is obtained by reaction of 4-(8-Methyl-8-aza-bicyclo[3.2.1]oct-3-endo)-yloxy)-benzotriazol-1-yl benzoate with 1-[4-(4-Ethylamino-phenoxy)-3-methoxy-phenyl]-3-(1-ethyl-propyl)-urea in the presence of 1 eq of DIEA.

### Example 44

### N-(4-{4-[3-(1-Ethyl-propyl)-ureido]-2-methoxy-phenoxy}-phenyl)-3-methoxy-4-(8-methyl-8-aza-bicyclo[3.2.1]oct-(3-endo)-yloxy)-benzamide

0.172 g of 1-[4-(4-Amino-phenoxy)-3-methoxy-phenyl]-3-(1-ethyl-propyl)-urea are placed in solution in 4 mL DMF with the 3-Methoxy-4-(8-methyl-8-aza-bicyclo[3.2.1]oct-(3-endo)-yloxy)-benzotriazol-1-yl benzoate obtained from 0.291 g of 3-Methoxy-4-(8-methyl-8-aza-bicyclo[3.2.1]oct-(3-endo)-yloxy)-benzoic acid such as described in Example 1, step A. After evaporating in vacuo at 60° C., the mixture is held in vacuo at 60° C. for 5 h and 15 h at AT. The residue is purified by semi-preparative HPLC. The desired product is isolated in TFA salt form following the operating mode described in Example 1.

Following the same operating mode as described in Example 44, the following compounds are obtained:

### 3-Chloro-N-(4-{4-[3-(1-ethyl-propyl)-ureido]-2-methoxy-phenoxy}-phenyl)-4-(8-methyl-8-aza-bicyclo[3.2.1]oct-(3-endo)-yloxy)-benzamide

### Example 45

The desired product is obtained by reaction of 3-Chloro(8-methyl-8-aza-bicyclo[3.2.1]oct-(3-endo)-yloxy)-benzotriazol-1-yle benzoate, isolated from 3-Chloro-4-(8-methyl-8-aza-bicyclo[3.2.1]oct-3-yloxy)-benzoic acid following the operating mode described in Example 1, step A, with 1-[4-(4-Amino-phenoxy)-3-methoxy-phenyl]-3-(1-ethyl-propyl)-urea.

### N-(4-{4-[3-(1-Ethyl-propyl)-ureido]-2-methoxy-phenoxy}-phenyl)-4-(8-methyl-8-aza-bicyclo[3.2.1]oct-6-yloxy)-benzamide

### Example 46

The desired product is obtained by reaction of 4-(8-Methyl-8-aza-bicyclo[3.2.1]oct-6-yloxy)-benzotriazol-1-yl benzoate, isolated from 4-(8-Methyl-8-aza-bicyclo[3.2.1]oct-6-yloxy)-benzoic acid following the operating mode described in Example 1, step A, with 1-[4-(4-Amino-phenoxy)-3-methoxy-phenyl]-3-(1-ethyl-propyl)-urea.

### N-(4-{4-[3-(1-Ethyl-propyl)-ureido]-2-methyl-phenoxy}-phenyl)-4-(8-methyl-8-aza-bicyclo[3.2.1]oct-(3-endo)-yloxy)-benzamide

### Example 47

The desired product is obtained by reaction of 4-(8-Methyl-8-aza-bicyclo[3.2.1]oct-(3-endo)-yloxy)-benzotriazol-1-yl benzoate with 1-[4-(4-Amino-2-methyl-phenoxy)-phenyl]-3-(1-ethyl-propyl)-urea.

### N-(4-{4-[3-(1-Ethyl-propyl)-ureido]-phenoxy}-2-fluoro-phenyl)-4-(8-methyl-8-aza-bicyclo[3.2.1]oct-(3-endo)-yloxy)-benzamide

### Example 48

The desired product is obtained by reaction of 4-(8-Methyl-8-aza-bicyclo[3.2.1]oct-(3-endo) yloxy)-benzotriazol-1-yl benzoate with 1-[4-(4-Amino-3-fluoro-phenoxy)-phenyl]-3-(1-ethyl-propyl)-urea.

### N-(4-{4-[3-(1-Ethyl-propyl)-ureido]-2-methoxy-phenoxy}-phenyl)-2-fluoro-4-(8-methyl-8-aza-bicyclo[3.2.1]oct-(3-endo)-yloxy)-benzamide

### Example 49

The desired product is obtained by reaction of 2-Fluoro 4-(8-methyl-8-aza-bicyclo[3.2.1]oct-(3-endo)-yloxy)-benzotriazol-1-yl benzoate, isolated from 2-Fluoro-4-(8-methyl-8-aza-bicyclo[3.2.1]oct-(3-endo)-yloxy)-benzoic acid following the operating mode described in Example 1, step A, with 1-[4-(4-Amino-phenoxy)-3-methoxy-phenyl]-3-(1-ethyl-propyl)-urea.

### N-(4-{2-Chloro-4-[3-(1-ethyl-propyl)-ureido]-phenoxy}-phenyl)-N-ethyl-4-(8-methyl-8-aza-bicyclo[3.2.1]oct-(3-endo)-yloxy)-benzamide

### Example 50

The desired product is obtained by reaction of 4-(8-Methyl-8-aza-bicyclo[3.2.1]oct-3-endo)-yloxy)-benzotriazol-1-yl benzoate with 1-[3-Chloro-4-(4-ethylamino-phenoxy)-phenyl]-3-(1-ethyl-propyl)-urea.

### Example 51

### N-(4-{4-[3-(1-Ethyl-propyl)-ureido]-2-methoxy-phenoxy}-phenyl)-4-(2,2,6,6-Tetramethyl-piperidin-4-yloxy)-benzamide

### A/ 4-(2,2,6,6-Tetramethyl-piperidin-4-yloxy)-benzotriazol-1-yl benzoate

A mixture of 0.500 g of 4-(2,2,6,6-Tetramethyl-piperidin-4-yloxy)-benzoic acid, 0.726 g of TBTU, 0.317 g of HOBT and 0.936 mL of DIEA in 20 mL DCM is stirred at AT for 1 h. The desired product is isolated following the operating mode described in Example 1, step A.

### B/ N-(4-{4-[3-(1-Ethyl-propyl)-ureido]-2-methoxy-phenoxy}-phenyl)-4-(2,2,6,6-Tetramethyl-piperidin-4-yloxy)-benzamide

The desired product is obtained in TFA salt form, from 0.275 g of 1-[4-(4-Amino-phenoxy)-3-methoxy-phenyl]-3-(1-ethyl-propyl)-urea and the compound of the preceding step, following the operating mode described in Example 24.

### Example 52

### N-(4-{4-[3-(1-Ethyl-propyl)-ureido]-2-methoxy-phenoxy}-phenyl)-4-(1,2,2,6,6-pentamethyl-piperidin-4-yloxy)-benzamide

### A/ 4-(1,2,2,6,6-Pentamethyl-piperidin-4-yloxy)-benzotriazol-1-yl benzoate

A mixture of 0.580 g of 4-(1,2,2,6,6-Pentamethyl-piperidin-4-yloxy)-benzoic acid, 0.700 g of TBTU, 0.297 g of HOBT and 1.10 mL of DIEA in 60 mL DCM is stirred at AT for 1 h. The desired product is isolated following the operating mode described in Example 1, step A.

### B/ N-(4-{4-[3-(1-Ethyl-propyl)-ureido]-2-methoxy-phenoxy}-phenyl)-4-(1,2,2,6,6-pentamethyl-piperidin-4-yloxy)-benzamide

The compound of the preceding step and 0.520 g of 1-[4-(4-Amino-phenoxy)-3-methoxy-phenyl]-3-(1-ethyl-propyl)-urea are placed in solution in 10 mL DMF, evaporated in vacuo at 60° C. and the mixture held in vacuo at 60° C. for 5 h and 48 h at AT. The residue is redissolved in DCM, the organic layer is washed with an aqueous HCl solution, with a sodium hydroxide solution, dried over MgSO₄, filtered and evaporated. The desired product is isolated in free base form after chromatography on silica eluting with a DCM/MeOH/NH₄OH mixture (98:2:0.1 v/v/v). The hydrochloride is obtained by treating with a HCl/diethyl ether mixture.

### Example 53

### N-(4-{4-[3-(1-Ethyl-propyl)-ureido]-2-methoxy-phenoxy}-phenyl)-4-(2-methyl-2-aza-bicyclo[2.2.2]oct-5-cis)-yloxy)-benzamide

### A/ 4-(2-Methyl-2-aza-bicyclo[2.2.2]oct-(5-cis)-yloxy)-benzotriazol-1-yl benzoate

0.520 g of 4-(2-Methyl-2-aza-bicyclo[2.2.2]oct-(5-cis)-yloxy)-benzoic acid, 0.280 g of TBTU, 0.120 g HOBT and 0.34 mL DIEA in 20 mL DCM is stirred at AT for 1 h. The desired product is isolated following the operating mode described in Example 1, step A.

### B/ N-(4-{4-[3-(1-Ethyl-propyl)-ureido]-2-methoxy-phenoxy}-phenyl)-4-(2-methyl-2-aza-bicyclo[2.2.2]oct-(5-cis)-yloxy)-benzamide

The desired product is obtained in TFA salt form, from 0.227 g of 1-[4-(4-Amino-phenoxy)-3-methoxy-phenyl]-3-(1-ethyl-propyl)-urea and the compound of the preceding step, following the operating mode described in Example 24.

### Example 54

### N-(5-{4-[3-(1-Ethyl-propyl)-ureido]-2-methoxymethyl-phenoxy}-thiazol-2-yl)-4-(1-isobutyl-1,2,3,6-Tetrahydro-pyridin-4-yl)-benzamide

### A/ 4-(1-Isobutyl-1,2,3,6-Tetrahydro-pyridin-4-yl)-benzotriazol-1-yl benzoate

A mixture of 0.940 g of 4-(1-Isobutyl-1,2,3,6-Tetrahydro-pyridin-4-yl)-benzoic acid, 1.110 g of TBTU, 0.487 g of HOBT and 2.30 mL of DIEA in 30 mL DCM is stirred at AT for 1 h. The desired product is isolated following the operating mode described in Example 1, step A.

### B/ N-(5-{4-[3-(1-Ethyl-propyl)-ureido]-2-methoxymethyl-phenoxy}-thiazol-2-yl)-4-(1-isobutyl-1,2,3,6-Tetrahydro-pyridin-4-yl)-benzamide

0.900 g of 1-[4-(2-Amino-thiazol-5-yloxy)-3-methoxymethyl-phenyl]-3-(1-ethyl-propyl)-urea are placed in solution in 10 ml DMF with the compound of the preceding step, heated at 65° C. for 15 h and evaporated in vacuo. The desired product is isolated in the form of a free base after chromatography on silica eluting with a DCM/MeOH/NH₄OH mixture (95:5:0.5 v/v/v) followed by precipitation with MeOH/pentane. The hydrochloride is obtained by treating with a HCl/diethyl ether mixture.

### Example 55

### N-(5-{4-[3-(1-Ethyl-propyl)-ureido]-2-methoxymethyl-phenoxy}-thiazol-2-yl)-4-(1-propyl-1,2,3,6-Tetrahydro-pyridin-(4-yl)-benzamide

### A/ 4-(1-Propyl-1,2,3,6-Tetrahydro-pyridin-4-yl)-benzotriazol-1-yl benzoate

A mixture of 0.200 g of 4-(1-Propyl-1,2,3,6-Tetrahydro-pyridin-4-yl)-benzoic acid, 0.249 g of TBTU, 0.107 g HOBT and 0.52 mL DIEA in 10 mL DCM is stirred at AT for 1 h. The desired product is isolated following the operating mode described in Example 1, step A.

### B/ N-(5-{4-[3-(1-Ethyl-propyl)-ureido]-2-methoxymethyl-phenoxy}-thiazol-2-yl)-4-(1-propyl-1,2,3,6-Tetrahydro-pyridin-4-yl)-benzamide

0.165 g of 1-[4-(2-Amino-thiazol-5-yloxy)-3-methoxymethyl-phenyl]-3-(1-ethyl-propyl)-urea are placed in solution in 5 ml DMF with the compound of the preceding step, heated to 65° C. for 5 h and evaporated in vacuo. The desired product is isolated in the form of a free base after chromatography on silica eluting with a DCM/MeOH/NH₄OH mixture (90:10:0.1 v/v/v) followed by precipitation with diethyl ether and washing with isopropanol and pentane. The hydrochloride is obtained by treating with a HCl/diethyl ether mixture.

### Example 56

### 4-(1-Butyl-1,2,3,6-Tetrahydro-pyridin-4-yl)-N-(5-{4-[3-(1-ethyl-propyl)-ureido]-2-methoxymethyl-phenoxy}-thiazol-2-yl)-benzamide

### A/ 4-(1-Butyl-1,2,3,6-Tetrahydro-pyridin-yl)-benzotriazol-1-yl benzoate

A mixture of 0.200 g of 4-(1-Butyl-1,2,3,6-Tetrahydro-pyridin-4-yl)-benzoic acid, 0.237 g of TBTU, 0.102 g of HOBT and 0.49 mL of DIEA in 10 mL DCM is stirred at AT for 1 h. The desired product is isolated following the operating mode described in Example 1, step A.

### B/ 4-(1-Butyl-1,2,3,6-Tetrahydro-pyridin-4-yl)-N-(5-{4-[3-(1-ethyl-propyl)-ureido]-2-methoxymethyl-phenoxy}-thiazol-2-yl)-benzamide

The desired product is obtained from 0.165 g of 1-[4-(2-Amino-thiazol-5-yloxy)-3-methoxymethyl-phenyl]-3-(1-ethyl-propyl)-urea and the compound of the preceding step, following the operating mode described in Example 55. The desired product is isolated in free base form.

### Example 57

### N-(5-{4-[3-(1-Ethyl-propyl)-ureido]-2-methoxymethyl-phenoxy}-thiazol-2-yl)-4-[1-(3-methyl-butyl)-1,2,3,6-Tetrahydro-pyridin-4-yl]-benzamide

### A/ 4-[1-(3-Methyl-butyl)-1,2,3,6-Tetrahydro-pyridin-4-yl]-benzotriazol-1-yl benzoate

A mixture of 0.200 g of 4-[1-(3-Methyl-butyl)-1,2,3,6-Tetrahydro-pyridin-4-yl]-benzoic acid, 0.226 g of TBTU, 0.097 g of HOBT and 0.47 mL of DIEA in 10 mL DCM is stirred at AT for 1 h. The desired product is isolated following the operating mode described in Example 1, step A.

### B/ N-(5-{4-[3-(1-Ethyl-propyl)-ureido]-2-methoxymethyl-phenoxy}-thiazol-2-yl)-4-[1-(3-methyl-butyl)-1,2,3,6-Tetrahydro-pyridin-4-yl]-benzamide

The desired product is obtained from 0.188 g of 1-[4-(2-Amino-thiazol-5-yloxy)-3-methoxymethyl-phenyl]-3-(1-ethyl-propyl)-urea and the compound of the preceding step, following the operating mode described in Example 55.

### Example 58

### N-5-{4-[3-(1-Ethyl-propyl)-ureido]-2-methoxymethyl-phenoxy}-thiazol-2-yl)-4-(1-isopropyl-piperidin-4-yl)-benzamide

### A/ 4-(1-Isopropyl-piperidin-4-yl)-benzotriazol-1-yl benzoate

A mixture of 0.500 g of 4-(1-Isopropyl-piperidin-4-yl)-benzoic acid, 0.620 g of TBTU, 0.260 g HOBT and 1.50 mL of DIEA in 10 mL DCM is stirred at AT for 1 h. The desired product is isolated following the operating mode described in Example 1, step A.

### B/ N-(5-{4-[3-(1-Ethyl-propyl)-ureido]-2-methoxymethyl-phenoxy}-thiazol-2-yl)-4-(1-isopropyl-piperidin-4-yl)-benzamide

The desired product is obtained from 0.410 g of 1-[4-(2-Amino-thiazol-5-yloxy)-3-methoxymethyl-phenyl]-3-(1-ethyl-propyl)-urea and the compound of the preceding step, following the operating mode described in Example 55, continuing the reaction for 48 h at AT after the heating step.

### Example 59

### 3-(4-Hydroxy-piperidin-1-ylmethyl)-1-isopropyl-1H-indole-6-carboxylic acid (5-{4-[3-(1-ethyl-propyl)-ureido]-2-methoxymethyl-phenoxy}-thiazol-2-yl)-amide

### A/ 3-(4-Hydroxy-piperidin-1-ylmethyl)-1-isopropyl-1H-indole-6-benzotriazol-1-yl carboxylate

A mixture of 0.316 g of 3-(4-Hydroxy-piperidin-1-ylmethyl)-1-isopropyl-1H-indole-6-carboxylic acid, 0.350 g TBTU, 0.150 g HOBT and 0.56 mL of DIEA in 30 mL DCM is stirred at AT for 0.5 h. The desired product is isolated following the operating mode described in Example 1, step A.

### B/ 3-(4-Hydroxy-piperidin-1-ylmethyl)-1-isopropyl-1H-indole-6-carboxylic acid (5-{4-[3-(1-ethyl-propyl)-ureido]-2-methoxymethyl-phenoxy}-thiazol-2-yl)-amide

The desired product is obtained from 0.255 g of 1-[4-(2-Amino-thiazol-5-yloxy)-3-methoxymethyl-phenyl]-3-(1-ethyl-propyl)-urea and the compound of the preceding step, following the operating mode described in Example 24.

### Example 60

### 2-[2-(4-Hydroxy-piperidin-1-yl)-ethyl]-benzofuran-6-carboxylic acid (5-{4-[3-(1-ethyl-propyl)-ureido]-2-methoxymethyl-phenoxy}-thiazol-2-yl)amide

### A/ 2-[2-(4-Hydroxy-piperidin-1-yl)-ethyl]-benzofuran-6-benzotriazol-1-yl carboxylate

A mixture of 0.860 g of 2-[2-(4-Hydroxy-piperidin-1-yl)-ethyl]-benzofuran-6-carboxylic acid, 0.481 g of TBTU, 0.203 g of HOBT and 0.73 mL of DIEA in 10 mL DCM is stirred at AT for 1 h. The desired product is isolated following the operating mode described in Example 1, step A.

### B/ 2-[2-(4-Hydroxy-piperidin-1-yl)-ethyl]-benzofuran-6-carboxylic acid (5-{4-[3-(1-ethyl-propyl)-ureido]-2-methoxymethyl-phenoxy}-thiazol-2-yl)-amide

0.300 g of 1-[4-(2-Amino-thiazol-5-yloxy)-3-methoxymethyl-phenyl]-3-(1-ethyl-propyl)-urea are placed in solution in 1.5 mL DMF with the compound of the preceding step, stirred at AT 15 h, evaporated in vacuo at 60° C. and the mixture held in vacuo at 60° C. for 4 h. The residue is purified by semi-preparative HPLC and then on a silica plate eluting with a DCM/MeOH/NH₄OH mixture (92:8:0.8 v/v/v). The silica is washed in methanol filtered, the filtrate evaporated under reduced pressure and precipitated in diethyl ether. The desired product is isolated in free base form.

### Example 61

### N-(5-{4-[3-(1-Ethyl-propyl)-ureido]-2-methoxy-phenoxy}-thiazol-2-yl)-4-(3-piperidin-1-yl-propoxy)-benzamide

Following General Procedure I, 75 mg of 4-(3-Piperidin-1-yl-propoxy)-benzoic acid are reacted in the presence of a TBTU/HOBT mixture for 10 min at AT, followed by the addition of 100 mg of 1-[4-(2-Amino-thiazol-5-yloxy)-3-methoxy-phenyl]-3-(1-ethyl-propyl)-urea, stirring for 48 h at AT, then evaporation in vacuo. The reaction medium is purified by semi-preparative HPLC. The desired product is isolated in TFA salt form, following the operating mode described in Example 1.

Following the same operating mode as described in Example 61, the following compounds are obtained:

### N-(4-{4-[3-(1-Ethyl-propyl)-ureido]-2-methoxy-phenoxy}-phenyl)-4-(1-isopropyl-piperidin-4-yloxy)-benzamide

### Example 62

The desired product is obtained from 4-(1-Isopropyl-piperidin-4-yloxy)-benzoic acid and 1-[4-(4-Amino-phenoxy)-3-methoxy-phenyl]-3-(1-ethyl-propyl)-urea.

### 4-(1-Butyl-piperidin-4-yloxy)-N-(4-{4-[3-(1-ethyl-propyl)-ureido]-2-methyl-phenoxy}-phenyl)-benzamide

### Example 63

The desired product is obtained from 4-(1-Butyl-piperidin-4-yloxy)-benzoic acid and 1-[4-(4-Amino-phenoxy)-3-methyl-phenyl]-3-(1-ethyl-propyl)-urea, the reaction of the amine being conducted for 2 h at 60° C. then at AT for 24 h.

### N-(4-{4-[3-(1-Ethyl-propyl)-ureido]-phenoxy}-3-methoxymethyl-phenyl)-4-(1-isopropyl-piperidin-4-yloxy)-benzamide

### Example 64

The desired product is obtained from 4-(1-Isopropyl-piperidin-4-yloxy)-benzoic acid and 1-[4-(4-Amino-2-methoxymethyl-phenoxy)-phenyl]-3-(1-ethyl-propyl)-urea.

### N-(4-{4-[3-(1-Ethyl-propyl)-ureido]-phenoxy}-3-methoxy-phenyl)-4-(1-isopropyl-piperidin-4-yloxy)-benzamide

### Example 65

The desired product is obtained from 4-(1-Isopropyl-piperidin-4-yloxy)-benzoic acid and 1-[4-(4-Amino-2-methoxy-phenoxy)-phenyl]-3-(1-ethyl-propyl)-urea, following the operating mode described in Example 63.

### N-5-{4-[3-(1-Ethyl-propyl)-ureido]-2-methylcarbamoylmethyl-phenoxy}-thiazol-2-yl)-4-(1-isobutyl-1,2,3,6-Tetrahydro-pyridin-4-yl)-benzamide

### Example 66

The desired product is obtained from 4-(1-Isobutyl-1,2,3,6-Tetrahydro-pyridin-4-yl)-benzoic acid and the amine 2-{2-(2-Amino-thiazol-5-yloxy)-5-[3-(1-ethyl-propyl)-ureido]-phenyl}-N-methyl-acetamide, following the operating mode described in Example 63.

### N-(4-{5-[3-(1-Ethyl-propyl)-ureido]-2-methoxy-phenoxy}-phenyl)-4-(1-isopropyl-piperidin-4-yloxy)-benzamide

### Example 67

The desired product is obtained from 4-(1-Isopropyl-piperidin-4-yloxy)-benzoic acid and 1-[3-(4-Amino-phenoxy)methoxy-phenyl]-3-(1-ethyl-propyl)-urea. The reaction medium is purified by semi-preparative HPLC. The desired product is isolated in HCl salt form, following the operating mode described in Example 19.

### {(4-cis)-[4-(4-{4-[3-(1-Ethyl-propyl)-ureido]-2-methoxy-phenoxy}-phenylcarbamoyl)-phenoxy]-cyclohexyl}-trimethyl-ammonium

### Example 68

The desired product is obtained from [(4-cis)-(4-Carboxy-phenoxy)-cyclohexyl]-trimethyl-ammonium and 1-[4-(4-Aminophenoxy)-3-methoxy-phenyl]-3-(1-ethyl-propyl)-urea following the operating mode described in Example 63.

### N-(4-{4-[3-(1-Ethyl-propyl)-ureido]-phenoxy}-3-methyl-phenyl)-4-(3-piperidin-1-yl-propoxy)-benzamide

### Example 69

The desired product is obtained from 4-(3-Piperidin-1-yl-propoxy)-benzoic acid and 1-[4-(4-Amino-2-methyl-phenoxy)-phenyl]-3-(1-ethyl-propyl)-urea.

### N-(5-{4-[3-(1-Ethyl-propyl)-ureido]-2-methoxymethyl-phenoxy}-thiazol-2-yl)-4-(1-isopropyl-piperidin-4-ylmethyl)-benzamide

### Example 70

The desired product is obtained from 4-(1-Isopropyl-piperidin ylmethyl)-benzoic acid and 1-[4-(2-Amino-thiazol-5-yloxy)-3-methoxymethyl-phenyl]-3-(1-ethyl-propyl)-urea.

### N-(5-{4-[3-(1-Ethyl-propyl)-ureido]-2-methoxymethyl-phenoxy}-thiazol-2-yl)-4-(1-isopropyl-piperidin-4-ylidenemethyl)-benzamide

### Example 71

The desired product is obtained from 4-(1-Isopropyl-piperidin-4-ylidenemethyl)-benzoic acid and 1-[4-(2-Amino-thiazol-5-yloxy)-3-methoxymethyl-phenyl]-3-(1-ethyl-propyl)-urea.

### 4-[1-(2-Dimethylamino-Acetyl)-1,2,3,6-Tetrahydro-pyridin-4-yl]-N-(5-{4-[3-(1-ethyl-propyl)-ureido]-2-methoxymethyl-phenoxy}-thiazol-2-yl)-benzamide

### Example 72

The desired product is obtained from 4-[1-(2-Dimethylamino-Acetyl)-1,2,3,6-Tetrahydro-pyridin-4-yl]-benzoic acid and 1-[4-(2-Amino-thiazol-5-yloxy)-3-methoxymethyl-phenyl]-3-(1-ethyl-propyl)-urea, the reaction of the amine being conducted for 16 h at 60° C. The desired product is isolated in TFA salt form, after chromatography on silica eluting with a DCM/MeOH/NH₄OH mixture (90:10:0.1 v/v/v), followed by semi-preparative HPLC.

### 1-Isopropyl-2-(2-piperidin-1-yl-ethyl)-1H-benzoimidazole-5-carboxylic acid (5-{4-[3-(1-ethyl-propyl)-ureido]-2-methoxy-phenoxy}-thiazol-2-yl)-amide

### Example 73

The desired product is obtained from 1-Isopropyl-2-(2-piperidin-1-yl-ethyl)-1H-benzoimidazole-5-carboxylic acid and 1-[4-(2-Amino-thiazol-5-yloxy)-3-methoxy-phenyl]-3-(1-ethyl-propyl)-urea.

### 1-Isopropyl-2-(2-piperidin-1-yl-ethyl)-1H-benzoimidazole-5-carboxylic acid (4-{4-[3-(1-ethyl-propyl)-ureido]-phenoxy}-3-methyl-phenyl)-amide

### Example 74

The desired product is obtained from 1-Isopropyl-2-(2-piperidin-1-yl-ethyl)-1H-benzoimidazole-5-carboxylic acid and 1-[4-(4-Amino-2-methyl-phenoxy)-phenyl]-3-(1-ethyl-propyl)-urea.

### 1-[3-(4-Hydroxy-piperidin-1-yl)-propyl]-1H-indole-5-carboxylic acid (5-{4-[3-(1-ethyl-propyl)-ureido]-2-methoxy-phenoxy}-thiazol-2-yl)-amide

### Example 75

The desired product is obtained from 1-[3-(4-Hydroxy-piperidin-1-yl)-propyl]-1H-indole-5-carboxylic acid and 1-[4-(2-Amino-thiazol-5-yloxy)-3-methoxy-phenyl]-3-(1-ethyl-propyl)-urea, the reaction of the amine being conducted for 2 h at 60° C. and 24 h at AT. The solvent is evaporated in vacuo, the reaction medium is kept in dry film at AT for 144 h and purified by semi-preparative HPLC.

### 1-(2-Piperidin-1-yl-ethyl)-1H-indole-5-carboxylic acid (5-{4-[3-(1-ethyl-propyl)-ureido]-2-methoxy-phenoxy}-thiazol-2-yl)-amide

### Example 76

The desired product is obtained from 1-(2-Piperidin 1-yl-ethyl)-1H-indole-5-carboxylic acid and 1-[4-(2-Amino-thiazol-5-yloxy)-3-methoxy-phenyl]-3-(1-ethyl-propyl)-urea.

### 4-[1,4′]Bipiperidin-1′-yl-N-(5-{4-[3-(1-ethyl-propyl)-ureido]-2-methoxy-phenoxy}-thiazol-2-yl)-benzamide

### Example 77

The desired product is obtained from 4-[1,4′]Bipiperidin-1′-yl-benzoic acid and 1-[4-(2-Amino-thiazol-5-yloxy)-3-methoxy-phenyl]-3-(1-ethyl-propyl)-urea following the operating mode described in Example 75, in the presence of 1.3 additional eq of the activating TBTU/HOBT mixture.

### 4-[Ethyl-(3-piperidin-1-yl-propionyl)-amino]-N-(5-{4-[3-(1-ethyl-propyl)-ureido]-2-methoxy-phenoxy}-thiazol-2-yl)-benzamide

### Example 78

The desired product is obtained from 4-[Ethyl-(3-piperidin-1-yl-propionyl)-amino]-benzoic acid and 1-[4-(2-Amino-thiazol-5-yloxy)-3-methoxy-phenyl]-3-(1-ethyl-propyl)-urea, in the presence of 1 additional eq of the activating TBTU/HOBT mixture. The DMF is evaporated in vacuo, the reaction medium kept in dry film at AT for 48 h, then purified by semi-preparative HPLC.

### 4-[Acetyl-(2-piperidin-1-yl-ethyl)-amino]-N-5-{4-[3-(1-ethyl-propyl)-ureido]-2-methoxy-phenoxy}-thiazol-2-yl)-benzamide

### Example 79

The desired product is obtained from 4-[Acetyl-(2-piperidin-1-yl-ethyl)-amino]-benzoic acid and 1-[4-(2-Amino-thiazol-5-yloxy)-3-methoxy-phenyl]-3-(1-ethyl-propyl)-urea.

### 4-[Ethyl-(3-piperidin-1-yl-propyl)-amino]-N-(5-{4-[3-(1-ethyl-propyl)-ureido]-2-methoxy-phenoxy}-thiazol-2-yl)-benzamide

### Example 80

The desired product is obtained from 4-[Ethyl-(3-piperidin-1-yl-propyl)-amino]-benzoic acid and 1-[4-(2-Amino-thiazol-5-yloxy)-3-methoxy-phenyl]-3-(1-ethyl-propyl)-urea, following the operating mode described in Example 78.

### N-(5-{4-[3-(1-Ethyl-propyl)-ureido]-2-methoxy-phenoxy}-thiazol-2-yl)-4-(3-piperidin-1-yl-propionylamino)-benzamide

### Example 81

The desired product is obtained from 4-(3-Piperidin-1-yl-propionylamino)-benzoic acid and 1-[4-(2-Amino-thiazol-5-yloxy)-3-methoxy-phenyl]-3-(1-ethyl-propyl)-urea, following the operating mode described in Example 78.

### 4-[Ethyl-(3-piperidin-1-yl-propionyl)-amino]-N-(5-{4-[3-(1-ethyl-propyl)-ureido]-phenoxy}-thiazol-2-yl)-benzamide

### Example 82

The desired product is obtained from 4-[Ethyl-(3-piperidin-1-yl-propionyl)-amino]-benzoic acid and 1-[4-(2-Amino-thiazol-5-yloxy)-phenyl]-3-(1-ethyl-propyl)-urea, the reaction of the amine being conducted for 24 h at AT, then 4 h at 80° C. in the presence of 1.5 additional eq of TBTU/HOBT activator mixture.

### 4-[Acetyl-(2-piperidin-1-yl-ethyl)-amino]-N-(4-{4-[3-(1-ethyl-propyl)-ureido]-phenoxy}-3-methyl-phenyl)-benzamide

### Example 83

The desired product is obtained from 4-[Acetyl(2-piperidin-1-yl-ethyl)-amino]-benzoic acid and 1-[4-(4-Amino-2-methyl-phenoxy)-phenyl]-3-(1-ethyl-propyl)-urea.

### 4-(4-Ethyl-piperazine-1-carbonyl)-N-(4-{4-[3-(1-ethyl-propyl)-ureido]-phenoxy}-3-methyl-phenyl)-benzamide

### Example 84

The desired product is obtained from 4-(4-Ethyl-piperazine-1-carbonyl)-benzoic acid and the amine 1-[4-(4-Amino-2-methyl-phenoxy)-phenyl]-3-(1-ethyl-propyl)-urea.

### 5-(3-Isopropyl-ureido)-2-(4-{[1-(2-piperidin-1-yl-ethyl)-1H-indole-5-carbonyl]-amino}-phenoxy)-methyl benzoate

### Example 85

The desired product is obtained following General Procedure J to react 1-(2-Piperidin-1-yl-ethyl)-1H-indole-5-carboxylic acid with 2-(4-Amino-phenoxy)-5-(3-isopropyl-ureido)-methyl benzoate.

### Example 86

### 4-(1-Butyl-piperidin-4-yloxy)-N-(4-{2-ethoxy-4-[3-(1-ethyl-propyl)-ureido]-phenoxy}-phenyl)-3-methoxy-benzamide

Following General Procedure 1,500 mg of 4-(1-Butyl-piperidin-4-yloxy)-3-methoxy-benzoic acid are activated in the presence of a TBTU/HOBT mixture for 30 min at AT, 570 mg of 1-[4-(4-Amino-phenoxy)-3-ethoxy-phenyl]-3-(1-ethyl-propyl)-urea are added, followed by stirring for 48 h at AT and evaporation in vacuo. The desired product is isolated in free base form after chromatography on silica eluting with a DCM/MeOH mixture (96:4 v/v). The hydrochloride is obtained by treating with a HCl/diethyl ether mixture.

Following the same operating mode as described in Example 86, the following compounds are obtained:

### 4-(1-Butyl-piperidin-4-yloxy)-N-(4-{4-[3-(1-ethyl-propyl)-ureido]-phenoxy}-2-fluoro-phenyl)-3-methyl-benzamide

### Example 87

The desired product is obtained from 4-(1-Butyl-piperidin-4-yloxy)-3-methyl-benzoic acid and 1-[4-(4-Amino-3-fluoro-phenoxy)-phenyl]-3-(1-ethyl-propyl)-urea. The desired product is isolated after chromatography on silica eluting with a DCM/MeOH mixture (98:2 v/v).

### N-(4-{4-[3-(1-Ethyl-propyl)-ureido]-2-methoxymethyl-phenoxy}-3-methyl-phenyl)-4-(1-isopropyl-piperidin-4-yloxy)-benzamide

### Example 88

The desired product is obtained from 4-(1-Isopropyl-piperidin-4-yloxy)-benzoic acid and 1-[4-(4-Amino-2-methyl-phenoxy)-3-methoxymethyl-phenyl]-3-(1-ethyl-propyl)-urea. The desired product is isolated after chromatography on silica eluting with a DCM/MeOH/NH₄OH mixture (95:5:0.5 v/v/v).

### 4-(1-Butyl-piperidin-4-yloxy)-N-ethyl-N-(4-{4-[3-(1-éethyl-propyl)-ureido]-2-methoxy-phenoxy}-phenyl)-benzamide

### Example 89

The desired product is obtained from 4-(1-Butyl-piperidin-4-yloxy)-benzoic acid and 1-[4-(4-Ethylamino-phenoxy)-3-methoxy-phenyl]-3-(1-ethyl-propyl)-urea, in the presence of 0.7 additional eq of acid and TBTU/HOBT activator mixture. The desired product is isolated after chromatography on silica eluting with a DCM/MeOH/NH₄OH mixture (95:5:0.5 v/v/v).

### N-(4-{4-[3-(1-Ethyl-propyl)-ureido]-2-methoxy-phenoxy}-phenyl)-4-(1-methyl-piperidin-4-yloxy)-benzamide

### Example 90

The desired product is obtained from 4-(1-Methyl-piperidin-4-yloxy)-benzoic acid and 1-[4-(4-Amino-phenoxy)-3-methoxy-phenyl]-3-(1-ethyl-propyl)-urea. The desired product is isolated after chromatography on silica eluting with a DCM/MeOH mixture (98:2 v/v).

### 4-(1-Butyl-piperidin-4-yloxy)-N-ethyl-N-(4-{4-[3-(1-ethyl-propyl)-ureido]-2-methoxy-phenoxy}-phenyl)-3-methoxy-benzamide

### Example 91

The desired product is obtained from 4-(1-Butyl-piperidin-4-yloxy)-3-methoxy-benzoic acid and 1-[4-(4-Ethylamino-phenoxy)-3-methoxy-phenyl]-3-(1-ethyl-propyl)-urea, in the presence of 1.3 additional eq of TBTU/HOBT activator mixture, for 15 h at 60° C. The desired product is isolated after two successive chromatographies on silica eluting with a DCM/MeOH/NH₄OH mixture (95:5:0.5 v/v/v).

### 4-(1-Butyl-piperidin-4-yloxy)-N-ethyl-N-(4-{4-[3-(1-ethyl-propyl)-ureido]-2-methoxy-phenoxy}-phenyl)-3-methyl-benzamide

### Example 92

The desired product is obtained from 4-(1-Butyl-piperidin-4-yloxy)-3-methyl-benzoic acid and 1-[4-(4-Ethylamino-phenoxy)-3-methoxy-phenyl]-3-(1-ethyl-propyl)-urea, in the presence of 1 additional eq of acid and TBTU/HOBT activator mixture, for 10 h at 60° C. and 15 h at TA. The desired product is isolated after chromatography on silica eluting with a DCM/MeOH/NH₄OH mixture (95:5:0.5 v/v/v).

### N-(5-{4-[3-(1-Ethyl-propyl)-ureido]-2-methoxymethyl-phenoxy}-thiazol-2-yl)-4-(1-isopropyl-1,2,3,6-Tetrahydro-pyridin-4-yl)-benzamide

### Example 93

The desired product is obtained from 4-(1-Isopropyl-1,2,3,6-Tetrahydro-pyridin-4-yl)-benzoic acid and 1-[4-(2-Amino-thiazol-5-yloxy)-3-methoxymethyl-phenyl]-3-(1-ethyl-propyl)-urea. The desired product is isolated after chromatography on silica eluting with a DCM/MeOH/NH₄OH mixture (95:5:0.5 v/v/v).

### 1-(3-Piperidin-1-yl-propyl)-1H-indole-5-carboxylic acid (4-{4-[3-(1-ethyl-propyl)-ureido]-2-methoxy-phenoxy}-3-methyl-phenyl)-amide

### Example 94

The desired product is obtained from 1-(3-Piperidin-1-yl-propyl)-1H-indole-5-carboxylic acid and 1-[4-(4-Amino-2-methyl-phenoxy)-3-methoxy-phenyl]-3-(1-ethyl-propyl)-urea. The desired product is isolated after chromatography on silica eluting with a DCM/MeOH/NH₄OH mixture (90:10:0.1 v/v/v), followed by precipitation with diethyl ether and pentane.

### 1-(2-Piperidin-1-yl-ethyl)-1H-indole-5-carboxylic acid (4-{4-[3-(1-ethyl-propyl)-ureido]-phenoxy}-3-methyl-phenyl)-amide

### Example 95

The desired product is obtained by following General Procedure J for the coupling of 1-(2-Piperidin-1-yl-ethyl)-1H-indole-5-carboxylic acid and 1-[4-(4-Amino-2-methyl-phenoxy)-phenyl]-3-(1-ethyl-propyl)-urea. The desired product is isolated in free base form after two successive chromatographies on silica eluting with a DCM/MeOH/NH₄OH mixture (95:5:0.5 v/v/v), followed by precipitation with pentane.

### Example 96

### 4-(1-Butyl-piperidin-4-yloxy)-N-(4-{4-[3-(1-ethyl-propyl)-ureido]-2-methoxy-phenoxy}-phenyl)-N-(2,2,2-trifluoro-ethyl)-benzamide

Following General Procedure K, 26 mg of 4-(1-Butyl-piperidin-4-yloxy)-benzoic acid are activated in the presence of PyClu for 10 min at AT, 39 mg of 1-(1-Ethyl-propyl)-3-{3-methoxy-4-[4-(2,2,2-trifluoro-ethylamino)-phenoxy]-phenyl}-urea are added, followed by heating under reflux for 2 h and evaporation in vacuo. The reaction medium is purified by semi-preparative HPLC. The desired product is isolated in TFA salt form, following the operating mode described in Example 1.

Following the same operating mode as described in Example 96, the following compound is obtained:

### N-(4-{4-[3-(1-Ethyl-propyl)-ureido]-2-methoxy-phenoxy}-phenyl)-4-(8-methyl-8-aza-bicyclo[3.2.1]oct-(3-endo)-yloxy)-N-(2,2,2-trifluoro-ethyl)-benzamide (Example 97

The desired product is obtained from 4-(8-Methyl-8-aza-bicyclo[3.2.1]oct-(3-endo)-yloxy)-benzoic acid and 1-(1-Ethyl-propyl)-3-{3-methoxy-4-[4-(2,2,2-trifluoro-ethylamino)-phenoxy]-phenyl}-urea.

### Example 98

### 4-(1-Butyl-piperidin-4-yloxy)-N-(4-{4-[3-(1-ethyl-propyl)-ureido]-2-methoxy-phenoxy}-phenyl)-3,5-dimethyl-benzamide

Following General Procedure M, 200 mg of 4-(1-Butyl-piperidin-4-yloxy)-3,5-dimethyl-benzoic acid are activated in the presence of TOTU for 15 min at AT, and coupled with 225 mg of 1-[4-(4-Amino-phenoxy)-3-methoxy-phenyl]-3-(1-ethyl-propyl)-urea. The solvent is evaporated in vacuo, and the desired product is isolated in free base form after chromatography on silica eluting with a DCM/MeOH mixture (95:5 v/v). The hydrochloride is obtained by treating with a HCl/diethyl ether mixture.

Following the same operating mode as described in Example 98, the following compounds are obtained:

### N-(4-{4-[3-(1-Ethyl-propyl)-ureido]-2-methoxy-phenoxy}-phenyl)-4-[1,(cis,cis-2,6)-trimethyl-piperidin-(cis-4)-yloxy]-benzamide

### Example 99

The desired product is obtained from 4-[1,(cis,cis-2,6-Trimethyl-piperidin-(cis-4)-yloxy]-benzoic acid and 1-[4-(4-Amino-phenoxy)-3-methoxy-phenyl]-3-ethyl-propyl)-urea. The desired product is isolated in free base form after chromatography on silica eluting with a DCM/MeOH/NH₄OH mixture (90:10:0.5 v/v/v). The hydrochloride is obtained by treating with a HCl/diethyl ether mixture.

### 2-Chloro-N-(4-{4-[3-(1-ethyl-propyl)-ureido]-2-methoxy-phenoxy}-phenyl)-4-(8-methyl-8-aza-bicyclo[3.2.1]oct-(3-endo)-yloxy)-benzamide

### Example 100

The desired product is obtained from 2-Chloro(8-methyl-8-aza-bicyclo[3.2.1]oct-(3-endo)-yloxy)-benzoic acid and 1-[4-(4-Amino-phenoxy)-3-methoxy-phenyl]-3-(1-ethyl-propyl)-urea. The desired product is isolated in hydrochloride form after purification of the reaction medium by semi-preparative HPLC, followed by treatment with a HCl/diethyl ether mixture, according to the operating mode described in Example 19.

### N-(4-{4-[3-(1-Ethyl-propyl)-ureido]-2-methoxy-phenoxy}-phenyl)-4-[1,(cis,cis-2,6)-trimethyl-piperidin-(trans-4)-yloxy]-benzamide

### Example 101

The desired product is obtained from 4-[1,(cis,cis-2,6)-Trimethyl-piperidin-(trans-4)-yloxy]-benzoic acid and 1-[4-(4-Amino-phenoxy)-3-methoxy-phenyl]-3-(1-ethyl-propyl)-urea. The desired product is isolated in TFA salt form, after purification of the reaction medium by semi-preparative HPLC, according to the operating mode described in Example 19.

### Example 102

### 4-(1-Butyl-piperidin-4-yloxy)-3-chloro-N-ethyl-N-(4-{4-[3-(1-ethyl-propyl)-ureido]-2-methoxy-phenoxy}-phenyl)-benzamide

200 mg of 4-(1-Butyl-piperidin-4-yloxy)-3-chloro-benzoic acid are reacted with 73 μL of oxalyl chloride in 3 ml DCM in the presence of a trace of DMF, at AT for 30 min. The reaction medium is evaporated in vacuo, 227 mg of 1-[4-(4-Ethylamino-phenoxy)-3-methoxy-phenyl]-3-(1-ethyl-propyl)-urea and 170 μL of TEA are added at 0° C., stirred at AT 15 h, and the solvent evaporated in vacuo. The desired product is isolated in free base form after chromatography on silica eluting with a DCM/MeOH/NH₄OH mixture (95:5:0.5 v/v/v). The hydrochloride is obtained by treating with a HCl/diethyl ether mixture.

### Example 103

### 4-(1-Butyl-piperidin-4-yloxy)-N-(4-{4-[3-(1-ethyl-propyl)-ureido]-2-methoxy-phenoxy}-phenyl)-benzamide

Following General Procedure L1, 7.60 g of 4-(1-Butyl-piperidin-4-yloxy)-benzoic acid are activated in the presence of an EDCI/HOBT mixture, 0.76 g of 1-[4-(4-Amino-phenoxy)-3-methoxy-phenyl]-3-(1-ethyl-propyl)-urea are added followed by stirring for 48 h at AT and evaporation of the solvent in vacuo. The desired product is isolated in free base form after chromatography on silica eluting with a DCM/MeOH/NH₄OH mixture (96:4:0.4 v/v/v). The hydrochloride is obtained after redissolving in MeOH and precipitation with 5 N HCl in isopropanol.

¹H NMR: 10.44 (s, 1H); 10.03 (s, 1H); 8.61 (s, 1H); 7.97 (m, 2H); 7.64-7.62 (d, 2H); 7.42 (s; 1H); 7.15-7.09 (m, 2H); 6.91-6.83 (m, 2H); 6.78-6.76 (d, 2H); 6.06-6.04 (d, 1H); 4.89-4.69 (m, 1H); 3.69 (s; 3H); 3.55-3.38 (m, 3H); 3.05 (m, 4H); 2.26-1.94 (m, 4H); 1.68 (m, 2H); 1.51-1.40 (m, 2H); 1.40-1.30 (m, 4H); 0.92 (t, 3H); 0.88 (t, 6H)

MS (APCI⁺): 603 (M+H)⁺

Elemental analysis: found C, 64.35. H, 7.33. N, 8.51. calculated for C₃₅H₄₆N₄O₅.1HCl.1H₂O C, 63.96. H, 7.51. N, 8.52

### Example 104

### 4-(1-Butyl-piperidin-4-yloxy)-N-(4-{4-[3-(1-ethyl-propyl)-ureido]-2-propoxy-phenoxy}-phenyl)-3-methyl-benzamide

Following General Procedure L1, 233 mg of 4-(1-Butyl-piperidin-4-yloxy)-3-methyl-benzoic acid are activated in the presence of an EDCI/HOBT mixture, 297 mg of 1-[4-(4-Amino-phenoxy)-3-propoxy-phenyl]-3-(1-ethyl-propyl)-urea are added followed by stirring for 48 h at AT and evaporation of the solvent in vacuo. The desired product is isolated in free base form after chromatography on silica eluting with a DCM/MeOH/NH₄OH mixture (95:5:0.5 v/v/v). The hydrochloride is obtained by treating with a HCl/diethyl ether mixture.

Following the same operating mode as described in Example 104, the following compounds are obtained:

### 4-(1-Butyl-piperidin-4-yloxy)-N-(4-{4-[3-(1-ethyl-propyl)-ureido]-2-meth phenoxy}-phenyl)-2-fluoro-benzamide

### Example 105

The desired product is obtained from 4-(1-Butyl-piperidin-4-yloxy)-2-fluoro-benzoic acid and 1-[4-(4-Amino-phenoxy)-3-methoxy-phenyl]-3-(1-ethyl-propyl)-urea, the activation of the acid and the coupling with the amine being conducted in DCM as solvent instead of DMF. The desired product is isolated after chromatography on silica eluting with a DCM/MeOH mixture (90:10 v/v).

### 4-(1-Butyl-piperidin-4-yloxy)-N-(4-{4-[3-(1-ethyl-propyl)-ureido]-phenoxy}-phenyl)-benzamide

### Example 106

The desired product is obtained from 4-(1-Butyl-piperidin-4-yloxy)-benzoic acid and 1-[4-(4-Amino-phenoxy)-phenyl]-3-(1-ethyl-propyl)-urea. The desired product is isolated after two successive chromatographies on silica.

### 1-(4-{1-[4-(1-Butyl-piperidin-4-yloxy)-benzoyl]-2,3-dihydro-1H-indol-5-yloxy}-3-methoxy-phenyl)-3-(1-ethyl-propyl)-urea

### Example 107

The desired product is obtained from 4-(1-Butyl-piperidin-4-yloxy)-benzoic acid and 1-[4-(2,3-Dihydro-1H-indol-5-yloxy)-3-methoxy-phenyl]-3-(1-ethyl-propyl)-urea. The desired product is isolated after chromatography on silica eluting with a DCM/MeOH/NH₄OH mixture (92:8:0.5 v/v/v).

### 4-(1-Butyl-piperidin-4-yloxy)-N-(4-{4-[3-(1-ethyl-propyl)-ureido]-2-methoxymethyl-phenoxy}-3-methyl-phenyl)-benzamide

### Example 108

The desired product is obtained from 4-(1-Butyl-piperidin-4-yloxy)-benzoic acid and 1-[4-(4-Amino-2-methyl-phenoxy)-3-methoxymethyl-phenyl]-3-(1-ethyl-propyl)-urea.

### N-(4-{4-[3-(1-Ethyl-propyl)-ureido]-phenoxy}-phenyl)-4-(1-isopropyl-piperidin-4-yloxy)-benzamide

### Example 109

The desired product is obtained from 4-(1-Isopropyl-piperidin-4-yloxy)-benzoic acid and 1-[4-(4-Amino-phenoxy)-phenyl]-3-(1-ethyl-propyl)-urea.

### 4-(1-Butyl-piperidin-4-yloxy)-N-(4-{4-[3-(1-ethyl-propyl)-ureido]-2-fluoro-phenoxy}-phenyl)-benzamide

### Example 110

The desired product is obtained from 4-(1-Butyl-piperidin-4-yloxy)-benzoic acid and 1-[4-(4-Amino-phenoxy)-3-fluoro-phenyl]-3-(1-ethyl-propyl)-urea. The desired product is isolated after chromatography on silica eluting with a DCM/MeOH/NH₄OH mixture (90:10:0.1 v/v/v).

### 1-(1-Ethyl-propyl)-3-(3-methoxy-4-{1-[4-(8-methyl-8-aza-bicyclo[3.2.1]oct-(3-endo)-yloxy)-benzoyl]-2,3-dihydro-1H-indol-5-yloxy}-phenyl)-urea

### Example 111

The desired product is obtained from 4-(8-Methyl-8-aza-bicyclo[3.2.1]oct-(3-endo)-yloxy)-benzoic acid and 1-[4-(2,3-Dihydro-1H-indol-5-yloxy)-3-methoxy-phenyl]-3-(1-ethyl-propyl urea. The desired product is isolated after chromatography on silica eluting with a DCM/MeOH/NH₄OH mixture (92:8:0.8 v/v/v).

### N-(4-{4-[3-(1-Ethyl-propyl)-ureido]-2-methoxy-phenoxy}-phenyl)-4-(8-methyl-8-aza-bicyclo[3.2.1]oct-(3-exo)-yloxy)-benzamide

### Example 112

The desired product is obtained from 4-(8-Methyl-8-aza-bicyclo[3.2.1]oct-(3-exo) yloxy)-benzoic acid and 1-[4-(4-Amino-phenoxy)-3-methoxy-phenyl]-3-(1-ethyl-propyl)-urea. The desired product is isolated in free base form after chromatography on silica eluting with a DCM/MeOH/NH₄OH mixture (90:10:0.1 v/v/v).

### N-(4-{2-Ethyl-4-[3-(1-ethyl-propyl)-ureido]-phenoxy}-phenyl)-4-(8-methyl-8-aza-bicyclo[3.2.1]oct-(3-endo)-yloxy)-benzamide

### Example 113

The desired product is obtained from 4-(8-Methyl-8-aza-bicyclo[3.2.1]oct-(3-endo)-yloxy)-benzoic acid and 1-[4-(4-Amino-phenoxy)-3-ethyl-phenyl]-3-(1-ethyl-propyl)-urea. The desired product is isolated in TFA salt form, after purification on silica followed by semi-preparative HPLC.

### 4-(1-Butyl-piperidin-4-yloxy)-N-{4-[4-(3-isopropyl-ureido)-phenoxy]-2-methoxy-phenyl}-benzamide

### Example 114

The desired product is obtained from 4-(1-Butyl-piperidin-4-yloxy)-benzoic acid and 1-[4-(4-Amino-3-methoxy-phenoxy)-phenyl]-3-isopropyl-urea. The desired product is isolated after chromatography on silica eluting with a DCM/MeOH/NH₄OH mixture (96:4:0.2 v/v/v).

### 1-(4-{1-[4-(1-Butyl-piperidin-4-yloxy)-3-methyl-benzoyl]-2,3-dihydro-1H-indol-5-yloxy}-3-methoxy-phenyl)-3-(1-ethyl-propyl)-urea

### Example 115

The desired product is obtained from 4-(1-Butyl-piperidin-4-yloxy)-3-methyl-benzoic acid and 1-[4-(2,3-Dihydro-1H-indol-5-yloxy)-3-methoxy-phenyl]-3-(1-ethyl-propyl)-urea.

### 4-(1-Butyl-piperidin-4-yloxy)-N-(4-{2-éthyl-4-[3-(1-ethyl-propyl)-ureido]-phenoxy}-phenyl)-benzamide

### Example 116

The desired product is obtained from 4-(1-Butyl-piperidin-4-yloxy)-benzoic acid and 1-[4-(4-Amino-phenoxy)-3-ethyl-phenyl]-3-(1-ethyl-propyl)-urea. The desired product is isolated after chromatography on silica eluting with a DCM/MeOH/NH₄OH mixture (90:10:0.1 v/v/v).

### N-(4-{4-[3-(1-Ethyl-propyl)-ureido]-2-methoxy-phenoxy}-phenyl)-4-(1-isopropyl-piperidin-4-ylmethoxy)-benzamide

### Example 117

The desired product is obtained from 4-(1-Isopropyl-piperidin-4-ylmethoxy)-benzoic acid and the amine 1-[4-(4-Amino-phenoxy)-3-methoxy-phenyl]-3-(1-ethyl-propyl)-urea. The desired product is isolated after chromatography on silica eluting with a DCM/MeOH/NH₄OH mixture (90:10:0.1 v/v/v).

### N-(4-{4-[3-(1-Ethyl-propyl)-ureido]-2-trifluoromethyl-phenoxy}-phenyl)-4-(8-methyl-8-aza-bicyclo[3.2.1]oct-(3-endo)-yloxy)-benzamide

### Example 118

The desired product is obtained from 4-(8-Methyl-8-aza-bicyclo[3.2.1]oct-(3-endo)-yloxy)-benzoic acid and 1-[4-(4-Amino-phenoxy)-3-trifluoromethyl-phenyl]-3-(1-ethyl-propyl)-urea.

### 4-(1-Butyl-piperidin-4-yloxy)-N-{4-[4-(3-dimethylamino-ureido)-phenoxy]-3-methoxymethyl-phenyl}-3-methyl-benzamide

### Example 119

The desired product is obtained from 4-(1-Butyl-piperidin-4-yloxy)-3-methyl-benzoic acid and 1-[4-(4-Amino-2-methoxymethyl-phenoxy)-phenyl]-3-dimethylamino-urea.

### 4-(1-Butyl-piperidin-4-yloxy)-N-(4-{4-[3-(N,N-dimethyl-amino)-ureido]-phenoxy}-3-methoxymethyl-phenyl)-3-methoxy-benzamide

### Example 120

The desired product is obtained from 4-(1-Butyl-piperidin-4-yloxy)-3-methoxy-benzoic acid and 1-[4-(4-Amino-2-methoxymethyl-phenoxy)-phenyl]-3-(N,N-dimethyl-amino)-urea. The desired product is isolated after chromatography on silica eluting with DCM/MeOH/NH₄OH mixture (85:15:0.5 v/v/v).

### 4-(1-Butyl-piperidin-4-yloxy)-N-(4-{4-[3-(1-ethyl-propyl)-ureido]-phenoxy}-3-trifluoromethyl-phenyl)-benzamide

### Example 121

The desired product is obtained from 4-(1-Butyl-piperidin-4-yloxy)-benzoic acid and 1-[4-(4-Amino-2-trifluoromethyl-phenoxy)-phenyl]-3-(1-ethyl-propyl)-urea. The desired product is isolated in TFA salt form, after purification by semi-preparative HPLC.

### 4-(1-Butyl-piperidin-4-yloxy)-N-{4-[4-(3-isopropyl-ureido)-benzyl]-phenyl}-benzamide

### Example 122

The desired product is obtained from 4-(1-Butyl-piperidin yloxy)-benzoic acid and 1-[4-(4-Amino-benzyl)-phenyl]-3-isopropyl-urea. The desired product is isolated after chromatography on silica eluting with a DCM/MeOH/NH₄OH mixture (96:4:0.2 v/v/v).

### Example 123

### N-(4-{4-[3-(1-Ethyl-propyl)-ureido]-phenoxy}-phenyl)-4-(8-methyl-8-aza-bicyclo[3.2.1]oct-(3-endo)-yloxy)-benzamide

Following General Procedure L2, 288 mg of 4-(8-Methyl-8-aza-bicyclo[3.2.1]oct-(3-endo)-yloxy)-benzoic acid are activated in the presence of an EDCI/HOBT mixture, followed by the addition of 188 mg of 1-[4-(4-Amino-phenoxy)-phenyl]-3-(1-ethyl-propyl)-urea, stirring for 16 h at AT then 4 h at 60° C., then evaporation in vacuo. The desired product is isolated in free base form after chromatography on silica eluting with a DCM/MeOH/NH₄OH mixture (95:5:0.5 v/v/v). The hydrochloride is obtained by treating with a HCl/diethyl ether mixture.

Following the same operating mode as described in Example 123, the following compounds are obtained:

### N-(4-{2-Chloro-4-[3-(1-ethyl-propyl)-ureido]-phenoxy}-phenyl)-4-(8-methyl-8-aza-bicyclo[3.2.1]oct-(3-endo)-yloxy)-benzamide

### Example 124

The desired product is obtained from 4-(8-Methyl-8-aza-bicyclo[3.2.1]oct-(3-endo)-yloxy)-benzoic acid and 1-[4-(4-Amino-phenoxy)-3-chloro-phenyl]-3-(1-ethyl-propyl)-urea. The desired product is isolated in TFA salt form after chromatography on silica followed by semi-preparative HPLC. The hydrochloride is obtained by following the operating mode described in Example 19.

### N-(4-{4-[3-(1-Ethyl-propyl)-ureido]-2-methoxymethyl-phenoxy}-phenyl)-4-(8-methyl-8-aza-bicyclo[3.2.1]oct-(3-endo)-yloxy)-benzamide

### Example 125

The desired product is obtained from 4-(8-Methyl-8-aza-bicyclo[3.2.1]oct-(3-endo)-yloxy)-benzoic acid and 1-[4-(4-Amino-phenoxy)-3-methoxymethyl-phenyl]-3-(1-ethyl-propyl)-urea.

### N-(4-{4-[3-(1-Ethyl-propyl)-ureido]-2-fluoro-phenoxy}-phenyl)-4-(8-methyl-8-aza-bicyclo[3.2.1]oct-(3-endo)-yloxy)-benzamide

### Example 126

The desired product is obtained from 4-(8-Methyl-8-aza-bicyclo[3.2.1]oct-(3-endo)-yloxy)-benzoic acid and 1-[4-(4-Amino-phenoxy)-3-fluoro-phenyl]-3-(1-ethyl-propyl)-urea. The desired product is isolated in TFA salt form, after semi-preparative HPLC. The hydrochloride is obtained following the operating mode described in Example 19.

### 4-(1-Butyl-piperidin-4-yloxy)-N-(4-{2-chloro-4-[3-(1-ethyl-propyl)-ureido]-phenoxy}-2-fluoro-phenyl)-benzamide

### Example 127

The desired product is obtained from 4-(1-Butyl-piperidin-4-yloxy)-benzoic acid and 1-[4-(4-Amino-3-fluoro-phenoxy)-3-chloro-phenyl]-3-(1-ethyl-propyl)-urea.

### Example 128

### 4-(1-Butyl-piperidin-4-yloxy)-N-(4-{2-ethoxy-4-[3-(1-ethyl-propyl)-ureido]-phenoxy}-phenyl)-3-methyl-benzamide

Following General Procedure L3, 200 mg of 4-(1-Butyl-piperidin-4-yloxy)-3-methyl-benzoic acid are reacted with 254 mg of 1-[4-(4-Amino-phenoxy)-3-ethoxy-phenyl]-3-(1-ethyl-propyl)-urea, in the presence of a EDCI/HOBT mixture. The solvent is evaporated in vacuo and the desired product is isolated in free base form after chromatography on silica eluting with a DCM/MeOH/NH₄OH mixture (95:5:0.5 v/v/v). The hydrochloride is obtained by treating with a HCl/diethyl ether mixture.

Following the same operating mode as described in Example 128, the following compounds are obtained:

### N-(4-{4-[3-(1-Ethyl-propyl)-ureido]-3-fluoro-phenoxy}-phenyl)-4-(8-methyl-8-aza-bicyclo[3.2.1]oct-(3-endo)-yloxy)-benzamide

### Example 129

The desired product is obtained from 4-(8-Methyl-8-aza-bicyclo[3.2.1]oct-(3-endo)-yloxy)-benzoic acid and 1-[4-(4-Amino-phenoxy)-2-fluoro-phenyl]-3-(1-ethyl-propyl)-urea.

### 4-(1-Butyl-piperidin-4-yloxy)-N-(4-{4-[3-(1-ethyl-propyl)-ureido]-2-methoxy-phenoxy}-phenyl)-3-fluoro-benzamide

### Example 130

The desired product is obtained from 4-(1-Butyl-piperidin-4-yloxy)-3-fluoro-benzoic acid and 1-[4-(4-Amino-phenoxy)-3-methoxy-phenyl]-3-1 ethyl-propyl)-urea. The desired product is isolated after chromatography on silica eluting with a DCM/MeOH mixture (90:10 v/v).

### 4-(1-Butyl-piperidin-4-yloxy)-N-(4-{4-[3-(1-ethyl-propyl)-ureido]-3-fluoro-phenoxy}-phenyl)-benzamide

### Example 131

The desired product is obtained from 4-(1-Butyl-piperidin-4-yloxy)-benzoic acid and 1-[4-(4-Amino-phenoxy)-2-fluoro-phenyl]-3-(1-ethyl-propyl)-urea.

### 4-(1-Butyl-piperidin-4-yloxy)-N-{4-[4-(3-isopropyl-ureido)-2-methoxy-phenoxy]-phenyl}-benzamide

### Example 132

The desired product is obtained from 4-(1-Butyl-piperidin-4-yloxy)-benzoic acid and 1-[4-(4-Amino-phenoxy)-3-methoxy-phenyl]-3-isopropyl-urea. The desired product is isolated in TFA salt form, after chromatography on silica eluting with a DCM/MeOH/NH₄OH mixture (90:10:0.1 v/v/v), followed by semi-preparative HPLC.

### 4-(1-Butyl-piperidin-4-yloxy)-N-(4-{4-[3-(1-ethyl-propyl)-ureido]-phenoxy}-2-fluoro-phenyl)-benzamide

### Example 133

The desired product is obtained from 4-(1-Butyl-piperidin-4-yloxy)-benzoic acid and 1-[4-(4-Amino-3-fluoro-phenoxy)-phenyl]-3-(1-ethyl-propyl)-urea. The desired product is isolated in free base form after chromatography on silica eluting with a DCM/MeOH/NH₄OH mixture (95:5:0.5 v/v/v).

### 4-(1-Butyl-piperidin-4-yloxy)-N-(4-{4-[3-(1-ethyl-propyl)-ureido]-2-methoxy-phenoxy}-phenyl)-3-trifluoromethyl-benzamide

### Example 134

The desired product is obtained from 4-(1-Butyl-piperidin-4-yloxy)-3-trifluoromethyl-benzoic acid and 1-[4-(4-Amino-phenoxy)-3-methoxy-phenyl]-3-(1-ethyl-propyl)-urea.

### 4-(1-Butyl-piperidin-4-yloxy)-N-(4-{4-[3-(1-ethyl-propyl)-ureido]-phenoxy}-2-methoxy-phenyl)-3-methyl-benzamide

### Example 135

The desired product is obtained from 4-(1-Butyl-piperidin-4-yloxy)-3-methyl-benzoic acid and the amine 1-[4-(4-Amino-3-methoxy-phenoxy)-phenyl]-3-(1-ethyl-propyl)-urea. The desired product is isolated in TFA salt form, after chromatography on silica eluting with a DCM/MeOH/NH₄OH mixture (95:5:0.1 v/v/v,) followed by semi-preparative HPLC. The hydrochloride is obtained following the operating mode described in Example 19.

### 4-(1-Butyl-piperidin-4-yloxy)-N-(4-{4-[3-(1-ethyl-propyl)-ureido]-phenoxy}-phenyl)-3-methyl-benzamide

### Example 136

The desired product is obtained from 4-(1-Butyl-piperidin-4-yloxy)-3-methyl-benzoic acid and 1-[4-(4-Amino-phenoxy)-phenyl]-3-(1-ethyl-propyl)-urea. The desired product is isolated after two successive chromatographies on silica eluting with a DCM/MeOH/NH₄OH mixture (95:5:0.1 v/v/v).

### N-(4-{4-[3-(1-Ethyl-propyl)-ureido]-phenoxy}-3-methyl-phenyl)-4-(8-methyl-8-aza-bicyclo[3.2.1]oct-(3-endo)-yloxy)-benzamide

### Example 137

The desired product is obtained from 4-(8-Methyl-8-aza-bicyclo[3.2.1]oct-(3-endo)-yloxy)-benzoic acid and 1-[4-(4-Amino-2-methyl-phenoxy)-phenyl]-3-(1-ethyl-propyl)-urea. The desired product is isolated after chromatography on silica eluting with a DCM/MeOH/NH₄OH mixture (90:10:0.1 v/v/v).

### 4-(1-Butyl-piperidin-4-yloxy)-N-{4-[4-(3-isopropyl-ureido)-phenoxy]-3,5-dimethyl-phenyl}-benzamide

### Example 138

The desired product is obtained from 4-(1-Butyl-piperidin-4-yloxy)-benzoic acid and 1-[4-(4-Amino-2,6-dimethyl-phenoxy)-phenyl]-3-isopropyl-urea. The desired product is isolated after two successive chromatographies on silica.

### N-(4-{3-[3-(1-Ethyl-propyl)-ureido]-phenoxy}-3-methyl-phenyl)-4-(1-isopropyl-piperidin-4-yloxy)-benzamide

### Example 139

The desired product is obtained from 4-(1-Isopropyl-piperidin-4-yloxy)-benzoic acid and 1-[4-(4-Amino-2-methyl-phenoxy)-phenyl]-3-(1-ethyl-propyl)-urea. The desired product is isolated after chromatography on silica eluting with a DCM/MeOH/NH₄OH mixture (90:10:0.01 v/v/v).

### 4-(1-Butyl-piperidin-4-yloxy)-N-{4-[4-(3-isopropyl-ureido)-phenoxy]-2,5-dimethyl-phenyl}-benzamide

### Example 140

The desired product is obtained from 4-(1-Butyl-piperidin-4-yloxy)-benzoic acid and 1-[4-(4-Amino-2,5-dimethyl-phenoxy)-phenyl]-3-isopropyl-urea. The desired product is isolated in free base form after chromatography on silica eluting with a DCM/MeOH/NH₄OH mixture (95:5:0.5 v/v/v).

### 4-(1-Butyl-piperidin-4-yloxy)-N-{4-[4-(3-isopropyl-ureido)-3-methoxy-phenoxy]-phenyl}-benzamide

### Example 141

The desired product is obtained from 4-(1-Butyl-piperidin-4-yloxy)-benzoic acid and 1-[4-(4-Amino-phenoxy)-2-methoxy-phenyl]-3-isopropyl-urea. The desired product is isolated in free base form, after chromatography on silica eluting with a DCM/MeOH/NH₄OH mixture (90:10:0.1 v/v/v).

### 4-(1-Isopropyl-1,2,3,6-Tetrahydro-pyridin-4-yl)-N-{4-[4-(3-isopropyl-ureido)-2-methoxy-phenoxy]-phenyl}-benzamide

### Example 142

The desired product is obtained from 4-(1-Isopropyl-1,2,3,6-Tetrahydro-pyridin-4-yl)-benzoic acid and 1-[4-(4-Amino-phenoxy)-3-methoxy-phenyl]-3-isopropyl-urea. The desired product is isolated after chromatography on silica eluting with a DCM/MeOH/NH₄OH mixture (90:1:0.1 v/v/v).

### (1-Ethyl-propyl)-carbamate of 4-{4-[4-(1-isopropyl-piperidin-4-yloxy)-benzoylamino]-2-methyl-phenoxy}-phenyle

### Example 143

The desired product is obtained from 4-(1-Isopropyl-piperidin-4-yloxy)-benzoic acid and (1-Ethyl-propyl)-carbamate of 4-(4-amino-2-methyl-phenoxy)-phenyl. The desired product is isolated in TFA salt form, after chromatography on silica eluting with a DCM/MeOH/NH₄OH mixture (90:10:0.1 v/v/v), followed by semi-preparative HPLC.

### N-(4-{4-[3-(1-Ethyl-propyl)-ureido]-2-methoxy-phenoxy}-phenyl)-4-(1-methyl-1,2,3,6-Tetrahydro-pyridin-4-yl)-benzamide

### Example 144

The desired product is obtained from 4-(1-Methyl-1,2,3,6-Tetrahydro-pyridin-4-yl)-benzoic acid and 1-[4-(1-Amino-phenoxy)-3-methoxy-phenyl]-3-(1-ethyl-propyl)-urea. The desired product is isolated in free base form, after chromatography on silica eluting with a DCM/MeOH mixture (95:5 v/v).

### N-(4-{4-[3-(1-Ethyl-propyl)-ureido]-2-methoxy-phenoxy}-3-methyl-phenyl)-4-(1-ethyl-1,2,3,6-Tetrahydro-pyridin-4-yl)-benzamide

### Example 145

The desired product is obtained from 4 (1-Ethyl-1,2,3,6-Tetrahydro-pyridin-4-yl)-benzoic acid and 1-[4-(4-Amino-2-methyl-phenoxy)-3-methoxy-phenyl]-3-(1-ethyl-propyl)-urea. The desired product is isolated in free base form after chromatography on silica eluting with a DCM/MeOH/NH₄OH mixture (95:5:0.5 v/v/v).

### 4-(1-Butyl-1,2,3,6-Tetrahydro-pyridin-4-yl)-N-(4-{4-[3-(1-ethyl-propyl)-ureido]-2-methoxy-phenoxy}-3-methyl-phenyl)-benzamide

### Example 146

The desired product is obtained from 4-(1-Butyl-1,2,3,6-Tetrahydro-pyridin-4-yl)-benzoic acid and 1-[4-(Amino-2-methyl-phenoxy)-3-methoxy-phenyl]-3-ethyl-propyl)-urea.

### 4-(1-Isobutyl-1,2,3,6-Tetrahydro-pyridin-4-yl)-N-{4-[4-(3-isopropyl-ureido)-2-methoxy-phenoxy]-phenyl}-benzamide

### Example 147

The desired product is obtained from 4-(1-Isobutyl-1,2,3,6-Tetrahydro-pyridin-4-yl)-benzoic acid and 1-[4-(4-Amino-phenoxy)-3-methoxy-phenyl]-3-isopropyl-urea.

### 1-(2-Piperidin-1-yl-ethyl)-1H-indole-5-carboxylic acid (4-{4-[3-(1-ethyl-propyl)-ureido]-2-methylcarbamoyl-phenoxy}-phenyl)-amide

### Example 148

The desired product is obtained from 1-(2-Piperidin-1-yl-ethyl)-1H-indole-5-carboxylic acid and 2-(4-Amino-phenoxy)-5-[3-(1-ethyl-propyl)-ureido]-N-methyl-benzamide. The desired product is isolated in free base form after chromatography on silica eluting with a DCM/MeOH/NH₄OH mixture (90:10:0.1 v/v/v).

### 4-[Acetyl-(3-piperidin-1-yl-propyl)-amino]-N-(4-{4-[3-(1-ethyl-propyl)-ureido]-phenoxy}-3-methyl-phenyl)-benzamide

### Example 149

The desired product is obtained from 4-[Acetyl-(3-piperidin-1-yl-propyl)-amino]-benzoic acid and 1-[4-(4-Amino-2-methyl-phenoxy)-phenyl]-3-(1-ethyl-propyl)-urea. The desired product is isolated in TFA salt form, after purification by semi-preparative HPLC.

### Example 150

### N-Ethyl-N-(4-{4-[3-(1-ethyl-propyl)-ureido]-2-methoxy-phenoxy}-phenyl)-3-fluoro-4-(8-methyl-8-aza-bicyclo[3.2.1]oct-(3-endo)-yloxy)-benzamide

Following General Procedure L4, 200 mg of 3-Fluoro-4-(8-methyl-8-aza-bicyclo[3.2.1]oct-(3-endo)-yloxy)-benzoic acid are reacted with 267 mg of 1-[4-(4-Ethylamino-phenoxy)-3-methoxy-phenyl]-3-(1-ethyl-propyl)-urea, in the presence of an EDCI/HOBT mixture. After evaporation in vacuo the desired product is isolated in free base form after chromatography on silica eluting with a DCM/MeOH/NH₄OH mixture (95:5:0.5 v/v/v). The hydrochloride is obtained by treating with a HCl/diethyl ether mixture.

Following the same operating mode as described in Example 150, the following compounds are obtained:

### N-(4-{4-[3-(1-Ethyl-propyl)-ureido]-2-methoxy-phenoxy}-phenyl)-3-fluoro-4-(8-methyl-8-aza-bicyclo[3.2.1]oct-(3-endo)-yloxy)-benzamide

### Example 151

The desired product is obtained from 3-Fluoro-4-(8-methyl-8-aza-bicyclo[3.2.1]oct-3-endo)-yloxy)-benzoic acid and 1-[4-(4-Amino-phenoxy)-3-methoxy-phenyl]-3-(1-ethyl-propyl)-urea.

### N-(4-{4-[3-(1-Ethyl-propyl)-ureido]-2-methoxy-phenoxy}-3-methyl-phenyl)-4-(1-methyl-1,2,3,6-Tetrahydro-pyridin-4-yl)-benzamide

### Example 152

The desired product is obtained from 4-(1-Methyl-1,2,3,6-Tetrahydro-pyridin-4-yl)-benzoic acid and from 1-[4-(4-Amino-2-methyl-phenoxy)-3-methoxy-phenyl]-3-(1-ethyl-propyl)-urea. The desired product is isolated in free base form, after chromatography on silica eluting with a DCM/MeOH/NH₄OH mixture (95:5:0.5 v/v/v).

### 4-(1-Butyl-piperidin-4-yloxy)-N-{4-[4-(3-isopropyl-ureido)-phenoxy]-phenyl}-benzamide

### Example 153

The desired product is obtained from 4-(1-Butyl-piperidin-4-yloxy)-benzoic acid and 1-[4-(4-Amino-phenoxy)-phenyl]-3-isopropyl-urea. The desired product is isolated in free base form.

### 4-[1-(2-Dimethylamino-Acetyl)-piperidin-4-yloxy]-N-(4-{4-[3-(1-ethyl-propyl)-ureido]-2-methoxy-phenoxy}-phenyl)-benzamide

### Example 154

The desired product is obtained from dimethylglycine and N-(4-{4-[3-(1-Ethyl-propyl)-ureido]-2-methoxy-phenoxy}-phenyl)-4-(piperidin-4-yloxy)-benzamide. The desired product is isolated after chromatography on silica eluting with a DCM/MeOH/NH₄OH mixture (90:1:0.1 v/v/v).

### 4-(1-Butyl-piperidin-4-yloxy-N-{4-[4-(2-dimethylamino-Acetylamino)-2-methoxy-phenoxy]-phenyl}-benzamide

### Example 155

The desired product is obtained from 4-(1-Butyl-piperidin-4-yloxy)-benzoic acid and N-[4-(4-Amino-phenoxy)-3-methoxy-phenyl]-2-dimethylamino-acetamide.

### 4-(1-Butyl-piperidin-4-yloxy)-N-{4-[3-hydroxymethyl-4-(3-isopropyl-ureido)-phenoxy]-phenyl}-benzamide

### Example 156

The desired product is obtained from 4-(1-Butyl-piperidin-4-yloxy)-benzoic acid and 1-[4-(4-Amino-phenoxy)-2-hydroxymethyl-phenyl]-3-isopropyl-urea. The desired product is isolated in free base form, after chromatography on silica eluting with a DCM/MeOH/NH₄OH mixture (90:10:0.2 v/v/v).

### 5-[3-(1-Ethyl-propyl)-ureido]-N-methyl-2-{4-[4-(3-piperidin-1-yl-propoxy)-benzoylamino]-phenoxy}-benzamide

### Example 157

The desired product is obtained from 4-(3-Piperidin-1-yl-propoxy)-benzoic acid and 2-(4-Amino-phenoxy)-5-[3-(1-ethyl-propyl)-ureido]-N-methyl-benzamide. The desired product is isolated in free base form after purification by semi-preparative HPLC, followed by chromatography on silica eluting with a DCM/MeOH/NH₄OH mixture (90:10:0.1 v/v/v).

### 4-[(4-cis)-Dimethylamino-cyclohexyloxy]-N-(4-{4-[3-(1-ethyl-propyl)-ureido]-2-methoxy-phenoxy}-phenyl)-benzamide

### Example 158

The desired product is obtained from 4-[(4-cis)-Dimethylamino-cyclohexyloxy]-benzoic acid and 1-[4-(4-Amino-phenoxy)-3-methoxy-phenyl]-3-(1-ethyl-propyl)-urea. The desired product is isolated in free base form.

### 5-[3-(1-Ethyl-propyl)-ureido]-2-(4-{4-[3-(4-hydroxy-piperidin-1-yl)-propoxy]-benzoylamino}-phenoxy)-N-methyl-benzamide

### Example 159

The desired product is obtained from 4-[3-(4-Hydroxy-piperidin-1-yl)propoxy]-benzoic acid and 2-(4-Amino-phenoxy)-5-[3-(1-ethyl-propyl)-ureido]-N-methyl-benzamide in the presence of 1.25 additional eq of acid and EDCI. The desired product is isolated in free base form, after purification by semi-preparative HPLC followed by chromatography on silica eluting with a DCM/MeOH/NH₄OH mixture (90:10:0.1 v/v/v).

### 4-(1-Butyl-piperidin-4-yloxy)-N-{4-[4-(3-isopropyl-ureido)-phenylsulfanyl]-phenyl}-benzamide

### Example 160

The desired product is obtained from 4-(1-Butyl-piperidin-4-yloxy)-benzoic acid and 1-[4-(4-Amino-phenylsulfanyl)-phenyl]-3-isopropyl-urea.

### N-(4-{4-[3-(1-Ethyl-propyl)-ureido]-3-fluoro-phenoxy}-phenyl)-4-(1-methyl-1,2,3,6-Tetrahydro-pyridin-4-yl)-benzamide

### Example 161

The desired product is obtained from 4-(1-Methyl-1,2,3,6-Tetrahydro-pyridin-4-yl)-benzoic acid and 1-[4-(4-Amino-phenoxy)-2-fluoro-phenyl]-3-(1-ethyl-propyl)-urea.

### N-(4-{4-[3-(1-Ethyl-propyl)-ureido]-2-methoxymethyl-phenoxy}-phenyl)-4-(1-isopropyl-1,2,3,6-Tetrahydro-pyridin-4-yl)-benzamide

### Example 162

The desired product is obtained from 4-(1-Isopropyl-1,2,3,6-Tetrahydro-pyridin-4-yl)-benzoic acid and 1-[4-(4-Amino-phenoxy)-3-methoxymethyl-phenyl]-3-(1-ethyl-propyl)-urea. The desired product is isolated in free base form.

### N-(4-{4-[3-(1-Ethyl-propyl)-ureido]-2-methoxy-phenoxy}-3-methyl-phenyl)-4-(1-isopropyl-1,2,3,6-Tetrahydro-pyridin-4-yl)-benzamide

### Example 163

The desired product is obtained from 4-(1-Isopropyl-1,2,3,6-Tetrahydro-pyridin-4-yl)-benzoic acid and 1-[4-(4-Amino-2-methyl-phenoxy)-3-methoxy-phenyl]-3-(1-ethyl-propyl)-urea. The desired product is isolated in TFA salt form, after chromatography on silica eluting with a DCM/MeOH/NH₄OH mixture (95:5:0.5 v/v/v), followed by semi-preparative HPLC.

### N-(4-{4-[3-(1-Ethyl-propyl)-ureido]-2-methoxy-phenoxy}-phenyl)-4-(1-isopropyl-1,2,3,6-Tetrahydro-pyridin-4-yl)-benzamide

### Example 164

The desired product is obtained from 4-(1-Isopropyl-1,2,3,6-Tetrahydro-pyridin-4-yl)-benzoic acid and 1-[4-(4-Amino-phenoxy)-3-methoxy-phenyl]-3-(1-ethyl-propyl)-urea. The desired product is isolated in TFA salt form, after chromatography on silica eluting with a DCM/MeOH/NH₄OH mixture (90:10:0.1 v/v/v), followed by semi-preparative HPLC.

### N-(4-{4-[3-(1-Ethyl-propyl)-ureido]-2-methoxy-phenoxy}-3-methyl-phenyl)-4-(1-propyl-1,2,3,6-Tetrahydro-pyridin-4-yl)-benzamide

### Example 165

The desired product is obtained from 4-(1-Propyl-1,2,3,6-Tetrahydro-pyridin-4-yl)-benzoic acid and 1-[4-(4-Amino-2-methyl-phenoxy)-3-methoxy-phenyl]-3-(1-ethyl-propyl)-urea. The desired product is isolated in free base form.

### 1-[3-(4-Hydroxy-piperidin-1-yl)-propyl]-1H-indole-5-carboxylic acid (4-{4-[3-(1-ethyl-propyl)-ureido]-2-methylcarbamoyl-phenoxy}-phenyl)-amide

### Example 166

The desired product is obtained from 1-[3-(4-Hydroxy-piperidin-1-yl)propyl]-1H-indole-5-carboxylic acid and 2-(4-Amino-phenoxy)-5-[3-(1-ethyl-propyl)-ureido]-N-methyl-benzamide, in the presence of 1 additional eq of acid, of EDCI and HOBT. The desired product is isolated in free base form after purification by semi-preparative HPLC, followed by chromatography on silica eluting with a DCM/MeOH/NH₄OH mixture (90:10:0.1 v/v/v).

### 1-(3-Piperidin-1-yl-propyl-1H-indole-5-carboxylic acid (4-{4-[3-(1-ethyl-propyl)-ureido]-phenoxy}-3-methyl-phenyl)-amide

### Example 167

The desired product is obtained from 1-(3-Piperidin-1-yl-propyl)-1H-indole-5-carboxylic acid 1-[4-(4-Amino-2-methyl-phenoxy)-phenyl]-3-(1-ethyl-propyl)-urea. The desired product is isolated in free base form, after chromatography on silica eluting with a DCM/MeOH/NH₄OH mixture (90:10:0.1 v/v/v).

### 3-Methyl-1-(2-piperidin-1-yl-ethyl)-1H-indole-5-carboxylic acid (4-{4-[3-(1-ethyl-propyl)-ureido]-phenoxy}-3-methyl-phenyl)-amide

### Example 168

The desired product is obtained from 3-Methyl-1-(2-piperidin-1-yl-ethyl)-1H-indole-5-carboxylic acid and 1-[4-(4-Amino-2-methyl-phenoxy)-phenyl]-3-(1-ethyl-propyl)-urea. The desired product is isolated in free base form after purification by semi-preparative HPLC, followed by chromatography on silica eluting with a DCM/MeOH/NH₄OH mixture (90:10:0.1 v/v/v).

### 3-Acetyl-1-(2-piperidin-1-yl-ethyl)-1H-indole-5-carboxylic acid (4-{4-[3-(1-ethyl-propyl)-ureido]-phenoxy}-3-methyl-phenyl)-amide (Example 169

The desired product is obtained from 3-Acetyl-1-(2-piperidin-1-yl-ethyl)-1H-indole-5-carboxylic acid and 1-[4-(4-Amino-2-methyl-phenoxy)-phenyl]-3-(1-ethyl-propyl)-urea in the presence of 0.5 additional eq of EDCI and HOBT. The desired product is isolated in free base form.

### 4-[Acetyl-(3-piperidin-1-yl-propyl)-amino]-N-(5-{4-[3-(1-ethyl-propyl)-ureido]-2-methoxy-phenoxy}-thiazol-2-yl)-benzamide

### Example 170

The desired product is obtained from 4-[Acetyl-(3-piperidin-1-yl-propyl)-amino]-benzoic acid and 1-[4-(2-Amino-thiazol-5-yloxy)-3-methoxy-phenyl]-3-(1-ethyl-propyl)-urea. The desired product is isolated in TFA salt form, after purification by semi-preparative HPLC.

### 2-(4-{4-[Acetyl-3-diethylamino-propyl)-amino]-benzoylamino}-phenoxy)-5-[3-(1-ethyl-propyl)-ureido]-N-methyl-benzamide

### Example 171

The desired product is obtained from 4-[Acetyl(3-diethylamino-propyl)-amino]-benzoic acid and 2-(4-Amino-phenoxy)-5-[3-(1-ethyl-propyl)-ureido]-N-methyl-benzamide. The desired product is isolated after chromatography on silica eluting with a DCM/MeOH/NH₄OH mixture (90:10:0.1 v/v/v).

### 4-[Ethyl-(3-piperidin-1-yl-propionyl)-amino]-N-(4-{4-[3-(1-ethyl-propyl)-ureido]-phenoxy}-3-methyl-phenyl)-benzamide

### Example 172

The desired product is obtained from 4-[Ethyl-(3-piperidin-1-yl-propionyl)-amino]-benzoic acid and 1-[4-(4-Amino-2-methyl-phenoxy)-phenyl]-3-(1-ethyl-propyl)-urea. The desired product is isolated is free base form.

### 4-[Ethyl-(3-piperidin-1-yl-propyl)-amino]-N-(4-{4-[3-(1-ethyl-propyl)-ureido]-phenoxy}-3-methyl-phenyl)-benzamide

### Example 173

The desired product is obtained from 4-[Ethyl-(3-piperidin-1-yl-propyl)-amino]-benzoic acid and 1-[4-(4-Amino-2-methyl-phenoxy)-phenyl]-3-(1-ethyl-propyl)-urea, in the presence of 1 additional eq of acid, EDCI and HOBT. The desired product is isolated in TFA salt form, after chromatography on silica eluting with a DCM/MeOH/NH₄OH mixture (90:10:0.1 v/v/v), followed by semi-preparative HPLC.

### N-(4-{4-[3-(1-Ethyl-propyl)-ureido]-phenoxy}-3-methyl-phenyl)-4-(3-piperidin-1-yl-propionylamino)-benzamide

### Example 174

The desired product is obtained from 4-(3-Piperidin-1-yl-propionylamino)-benzoic acid and 1-[4-(4-Amino-2-methyl-phenoxy)-phenyl]-3-(1-ethyl-propyl)-urea. The desired product is isolated in free base form after chromatography on silica eluting with a DCM/MeOH/NH₄OH mixture (90:10:0.1 v/v/v).

### 1-(3-Piperidin-1-yl-propyl)-1H-indole-5-carboxylic acid (4-{4-[3-(1-ethyl-propyl)-ureido]-2-methylcarbamoyl-phenoxy}-phenyl)-amide

### Example 175

The desired product is obtained from 1-(3-piperidin-1-yl-propyl-1H-indole-5-carboxylic acid and 2-(4-Amino-phenoxy)-5-[3-(1-ethyl-propyl)-ureido]-N-methyl-benzamide. The desired product is isolated in TFA salt form, after chromatography on silica eluting with a DCM/MeOH/NH₄OH mixture (90:10:0.1 v/v/v), followed by semi-preparative HPLC.

### Example 176

### 4-(1-Benzyl-piperidin-4-yloxy)-N-(4-{4-[3-(1-ethyl-propyl)-ureido]-2-methoxy-phenoxy}-phenyl)-benzamide

The desired product is obtained following the operating mode described under Preparation 119, step A.

### Example 177

### N-(4-{4-[3-(1-Ethyl-propyl)-ureido]-2-methoxy-phenoxy}-phenyl)-4-(piperidin-4-yloxy)-benzamide

The desired product is isolated in TFA salt form after purification by semi-preparative HPLC of 200 mg of compound obtained such as described under Preparation 119.

### Example 178

### N-(4-{4-[3-(1-Ethyl-propyl)-ureido]-phenoxy}-3-methyl-phenyl)-4-(piperidin-4-yloxy)-benzamide

The desired product is isolated in TFA salt from after purification by semi-preparative HPLC of 200 mg of compound obtained such as described under Preparation 118.

### Example 179

### N-(4-{4-[3-(1-Ethyl-propyl)-ureido]-2-methoxy-phenoxy}-phenyl)-4-(1-propyl-piperidin-4-yloxy)-benzamide

172 mg of N-(4-{4-[3-(1-Ethyl-propyl)-ureido]-2-methoxy-phenoxy}-phenyl)-4-(piperidin-4-yloxy)-benzamide are placed in solution in DMF (1.5 ml DMF/0.1 mmol amine), followed by the addition of 2.2 eq of DIEA and 1.2 eq of 1-bromopropane and heating at 80° C. for 72 h. After evaporation in vacuo, the desired product is isolated in TFA salt form, after purification by semi-preparative HPLC, following the operating mode described in Example 1.

Following the same operating mode as described in Example 179, the following compounds are obtained:

### 4-{4-[4-(4-{4-[3-(1-ethyl-propyl)-ureido]-2-methoxy-phenoxy}-phenylcarbamoyl)-phenoxy]-piperidin-1-yl}-butyl acetate

### Example 180

The desired product is obtained from N-(4-{4-[3-(1-Ethyl-propyl)-ureido]-2-methoxy-phenoxy}-phenyl)-4-(piperidin-4-yloxy)-benzamide and 4-bromobutyl acetate.

### 4-[1-(3-Dimethylamino-propyl)-piperidin-4-yloxy]-N-(4-{4-[3-(1-ethyl-propyl)-ureido]-2-methoxy-phenoxy}-phenyl)-N-(2-methoxy-ethyl)-benzamide

### Example 181

The desired product is obtained from N-(4-{4-[3-(1-Ethyl-propyl)-ureido]-2-methoxy-phenoxy}-phenyl)-N-(2-methoxy-ethyl)-4-(piperidin-4-yloxy)-benzamide and 3.6 eq of 3-dimethylamino-1-propyl chloride in the presence of 7.2 eq of DIEA, by heating for 24 h at 80° C.

### 4-[1-(3-Dimethylamino-propyl)-piperidin-4-yloxy]-N-(4-{4-[3-(1-ethyl-propyl)-ureido]-2-methoxy-phenoxy}-phenyl)-N-isobutyl-benzamide

### Example 182

The desired product is obtained from N-(4-{4-[3-(1-Ethyl-propyl)-ureido]-2-methoxy-phenoxy}-phenyl)-N-isobutyl-4-(piperidin-4-yloxy)-benzamide and 3.6 eq of 3-dimethylamino-1-propyl chloride in the presence of 7.2 eq of DIEA, by heating for 3 h at 80° C.

### 4-(1-Butyl-piperidin-4-yloxy)-N-(4-{4-[3-(1-ethyl-propyl)-ureido]-2-methoxy-phenoxy}-3-methyl-phenyl)-benzamide

### Example 183

The desired product is obtained from N-(4-{4-[3-(1-Ethyl-propyl)-ureido]-2-methoxy-phenoxy}-3-methyl-phenyl)-4-(piperidin-4-yloxy)-benzamide and 1-bromobutane, by heating at 90° C. for 15 h.

### 4-(8-Butyl-8-aza-bicyclo[3.2.1]oct-(3-endo)-yloxy)-N-(4-{4-[3-(1-ethyl-propyl)-ureido]-2-methoxy-phenoxy}-phenyl)-benzamide

### Example 184

The desired product is obtained from 4-(8-Aza-bicyclo[3.2.1]oct-(3-endo)-yloxy)-N-(4-{4-[3-(1-ethyl-propyl)-ureido]-2-methoxy-phenoxy}-phenyl)-benzamide and 1-bromobutane by heating at 80° C. for 15 h. The reaction medium is purified by semi-preparative HPLC in an ammonium bicarbonate medium, the solvent is evaporated in vacuo, the residue solubilized in DCM and the organic layer is washed with water, dried over MgSO₄, filtered and treated with a HCl/diethyl ether mixture. The desired product is isolated in hydrochloride form after evaporation in vacuo of the organic layer and precipitation of the residue with diethyl ether.

### 4-(8-Ethyl-8-aza-bicyclo[3.2.1]oct-(3-endo)-yloxy)-N-(4-{4-[3-(1-ethyl-propyl)-ureido]-2-methoxy-phenoxy}-phenyl)-benzamide

### Example 185

The desired product is obtained from 4-(8-Aza-bicyclo[3.2.1]oct-(3-endo)-yloxy)-N-(4-{4-[3-(1-ethyl-propyl)-ureido]-2-methoxy-phenoxy}-phenyl)-benzamide and bromoethane following the operating mode described in Example 184.

### N-(4-{4-[3-(1-Ethyl-propyl)-ureido]-2-methoxy-phenoxy}-phenyl)-4-[8-(3-methoxy-propyl)-8-aza-bicyclo[3.2.1]oct-(3-endo)-yloxy]-benzamide

### Example 186

The desired product is obtained from 4-(8-Aza-bicyclo[3.2.1]oct-(3-endo)-yloxy)-N-(4-{4-[3-(1-ethyl-propyl)-ureido]-2-methoxy-phenoxy}-phenyl)-benzamide and 1-bromo-3-methoxy-propane following the operating mode described in Example 184.

### N-(4-{4-[3-(1-Ethyl-propyl)-ureido]-2-methoxy-phenoxy}-phenyl)-4-[8-(2-methoxy-ethyl)-8-aza-bicyclo[3.2.1]oct-(3-endo)-yloxy]-benzamide

### Example 187

The desired product is obtained from 4-(8-Aza-bicyclo[3.2.1]oct-(3-endo)-yloxy)-N-(4-{4-[3-(1-ethyl-propyl)-ureido]-2-methoxy-phenoxy}-phenyl)-benzamide and 2-bromoethyl methyl ether following the operating mode described in Example 184.

### N-(4-{4-[3-(1-Ethyl-propyl)-ureido]-2-methoxy-phenoxy}-phenyl)-4-[1-(4,4,4-trifluoro-butyl)-piperidin-4-yloxy]-benzamide

### Example 188

The desired product is obtained from N-(4-{4-[3-(1-Ethyl-propyl)-ureido]-2-methoxy-phenoxy}-phenyl piperidin-4-yloxy)-benzamide and 4,4,4-trifluoro-1-bromobutane, following the operating mode described in Example 179.

### 4-[1-(2-Diethylamino-ethyl)-piperidin-4-yloxy]-N-(4-{4-[3-(1-ethyl-propyl)-ureido]-2-methoxy-phenoxy}-3-methyl-phenyl)-benzamide

### Example 189

The desired product is obtained from N-(4-{4-[3-(1-Ethyl-propyl)-ureido]-2-methoxy-phenoxy}-3-methyl-phenyl)-4-(piperidin-4-yloxy)-benzamide and 2-bromo-N,N-diethylamine following the operating mode described in Example 179, after heating at 90° C. for 15 h.

### N-(4-{4-[3-(1-Ethyl-propyl)-ureido]-2-methoxy-phenoxy}-phenyl)-4-[1-(4-fluoro-butyl-piperidin-4-yloxy]-benzamide

### Example 190

The desired product is obtained from N-(4-{4-[3-(1-Ethyl-propyl)-ureido]-2-methoxy-phenoxy}-phenyl)-4-(piperidin-4-yloxy)-benzamide and 1-bromofluorobutane, following the operating mode described in Example 179.

### 4-[1-(1-Ethyl-propel)-piperidin-4-yloxy]-N-(4-{4-[3-(1-ethyl-propel)-ureido]-phenoxy}-3-methyl-phenyl)-benzamide

### Example 191

The desired product is obtained from N-{4-(4-[3-(1-Ethyl-propyl)-ureido]-phenoxy}-3-methyl-phenyl)-4-(piperidin-4-yloxy)-benzamide and 3 eq of 3-bromopentane, following the operating mode described in Example 179.

### {4-[4-(4-{4-[3-(1-Ethyl-propyl)-ureido]-phenoxy}-3-methyl-phenylcarbamoyl)-phenoxy]-piperidin-1-yl}-ethyl acetate

### Example 192

The desired product is obtained from N-(4-{4-[3-(1-Ethyl-propyl)-ureido]-phenoxy}-3-methyl-phenyl)-4-(piperidin-4-yloxy)-benzamide and 1 eq of ethyl bromoacetate in the presence of 1 eq of DIEA, following the operating mode described in Example 179, after heating at 90° C. for 24 h.

### Example 193

### {4-[4-(4-{4-[3-(1-Ethyl-propyl)-ureido]-phenoxy}-3-methyl-phenylcarbamoyl)-phenoxy]-piperidin-1-yl}-acetic acid

90 mg of compound obtained such as described in Example 192 are heated under reflux for 1 h in a mixture of 37% aqueous HCl (6 ml)/acetone (2 ml), then evaporated in vacuo. The desired product is isolated in TFA salt form, after purification of the residue by semi-preparative HPLC, following the operating mode described in Example 1.

### Example 194

### N-(4-{4-[3-(1-Ethyl-propyl-ureido]-2-methoxy-phenoxy}-phenyl)-4-[1-(4-hydroxy-butyl)-piperidin-4-yloxy]-benzamide

80 mg of compound obtained such as described in Example 180 are heated under reflux for 5 h in a mixture of 1 N aqueous NaOH (5 ml)/MeOH (1 ml), then evaporated in vacuo. The desired product is isolated in TFA salt form, after purification of the residue by semi-preparative HPLC, following the operating mode described in Example

### Example 195

### 4-{(3-endo)-[4-(4-{4-[3-(1-ethyl-propyl)-ureido]-2-methoxy-phenoxy}-phenylcarbamoyl)-phenoxy]-8-aza-bicyclo[3.2.1]oct-8-yl}-butyl acetate

240 mg of 4-(8-Aza-bicyclo[3.2.1]oct-(3-endo)-yloxy)-N-(4-{4-[3-ethyl-propyl)-ureido]-2-methoxy-phenoxy}-phenyl)-benzamide are placed in solution in DMF (1.5 ml DMF/0.1 mmol amine), followed by the addition of 2.2 eq of DIEA and 1.2 eq of 4-bromobutyl acetate, heating at 80° C. for 15 h, then the addition of 2.4 eq of halide and 4.4 eq of DIEA, and heating for a further 15 h at 80° C. After evaporation in vacuo, the desired product is isolated in TFA salt form, after purification of the residue by semi-preparative HPLC, following the operating mode described in Example 1.

Following the same operating mode as described in Example 195, the following compounds are obtained:

### 4-[8-(3-Dimethylamino-propyl)-8-aza-bicyclo[3.2.1]oct-(3-endo)-yloxy]-N-(4-{4-[3-(1-ethyl-propyl)-ureido]-2-methoxy-phenoxy}-phenyl)-benzamide

### Example 196

The desired product is obtained from 4-(8-Aza-bicyclo[3.2.1]oct-(3-endo)-yloxy)-N-(4-{4-[3-(1-ethyl-propyl)-ureido]-2-methoxy-phenoxy}-phenyl)-benzamide and 3-dimethylamino-1-propyl chloride.

### 4-[1-(3-Dimethylamino-propyl)-piperidin-4-yloxy]-N-(4-{4-[3-(1-ethyl-propyl)-ureido]-2-methoxy-phenoxy}-phenyl)-benzamide

### Example 197

The desired product is obtained from N-(4-{4-[3-(1-Ethyl-propyl)-ureido]-2-methoxy-phenoxy}-phenyl)-4-(piperidin-4-yloxy)-benzamide and 3-dimethylamino-1-propyl chloride. After evaporation in vacuo, the desired product is isolated in free base form, after chromatography on silica eluting with a DCM/MeOH/NH₄OH mixture (90:10:0.1 v/v/v). The hydrochloride is obtained by treating with a HCl/diethyl ether mixture.

### N-(4-{4-[3-(1-Ethyl-propyl)-ureido]-2-methoxy-phenoxy}-phenyl)-4-(8-propyl-8-aza-bicyclo[3.2.1]oct-3-yloxy)-benzamide

### Example 198

The desired product is obtained from 4-(8-Aza-bicyclo[3.2.1]oct-3-yloxy)-N-(4-{4-[3-(1-ethyl-propyl)-ureido]-2-methoxy-phenoxy}-phenyl)-benzamide and from 1-bromopropane, an additional 1.2 eq of halide and an additional 2.2 eq of DIEA being added during the second heating step. The desired product is isolated in hydrochloride form after purification by semi-preparative HPLC in an ammonium bicarbonate medium, according to the treatment described in Example 184.

### 4-(1-sec-Butyl-piperidin-4-yloxy)-N-(4-{4-[3-(1-ethyl-propyl)-ureido]-2-methoxy-phenoxy}-phenyl)-benzamide

### Example 199

The desired product is obtained from N-(4-{4-[3-(1-Ethyl-propyl)-ureido]-2-methoxy-phenoxy}-phenyl)-4-(piperidin-4-yloxy)-benzamide and from 2-bromobutane, an additional 1.2 eq of halide and additional 2.2 eq of DIEA being added during the second heating step, which lasts 48 h.

### 4-(1-Butyl-piperidin-4-yloxy)-N-(4-{4-[3-(1-ethyl-propyl)-ureido]-2-meth phenoxy}-phenyl)-N-(2-methoxy-ethyl)-benzamide

### Example 200

The desired product is obtained from N-(4-{4-[3-(1-Ethyl-propyl)-ureido]-2-methoxy-phenoxy}-phenyl)-N-(2-methoxy-ethyl)-4-(piperidin-4-yloxy)-benzamide and from 1-bromobutane, 1 additional eq of halide and 1 additional eq of DIEA being added during the second heating step.

### N-(4-{4-[3-(1-Ethyl-propyl)-ureido]-2-methoxy-phenoxy}-phenyl)-4-[8-(2-hydroxy-ethyl)-8-aza-bicyclo[3.2.1]oct-(3-endo)-yloxy]-benzamide

### Example 201

The desired product is obtained from 4-(8-Aza-bicyclo[3.2.1]oct-(3-endo)-yloxy)-N-(4-{4-[3-(1-ethyl-propyl)-ureido]-2-methoxy-phenoxy}-phenyl)-benzamide and from 2-bromoethanol.

### N-(4-{4-[3-(1-Ethyl-propyl)-ureido]-2-methoxy-phenoxy}-phenyl)-4-[8-(4,4,4-trifluoro-butyl)-8-aza-bicyclo[3.2.1]oct-(3-endo)-yloxy]-benzamide

### Example 202

The desired product is obtained from 4-(8-Aza-bicyclo[3.2.1]oct-(3-endo)-yloxy)-N-(4-{4-[3-(1-ethyl-propyl)-ureido]-2-methoxy-phenoxy}-phenyl)-benzamide and 4,4,4-trifluoro-1-bromobutane, an additional 0.5 eq of halide and of DIEA being added during the second heating step which lasts 24 h.

### N-(4-{4-[3-(1-Ethyl-propyl)-ureido]-2-methoxy-phenoxy}-phenyl)-4-[8-(3-hydroxy-propyl)-8-aza-bicyclo[3.2.1]oct-(3-endo)-yloxy]-benzamide

### Example 203

The desired product is obtained from 4-(8-Aza-bicyclo[3.2.1]oct-(3-endo) yloxy)-N-(4-{4-[3-(1-ethyl-propyl)-ureido]-2-methoxy-phenoxy}-phenyl)-benzamide and 3-bromo-1-propanol.

### N-(4-{4-[3-(1-Ethyl-propyl)-ureido]-2-methoxy-phenoxy}-phenyl)-4-(8-isopropyl-8-aza-bicyclo[3.2.1]oct-(3-endo)-yloxy)-benzamide

### Example 204

The desired product is obtained from 4-(8-Aza-bicyclo[3.2.1]oct-(3-endo)-yloxy)-N-(4-{4-[3-(1-ethyl-propyl)-ureido]-2-methoxy-phenoxy}-phenyl)-benzamide and 2-bromopropane.

### 4-(1-Cyclohexylmethyl-piperidin-4-yloxy)-N-(4-{4-[3-(1-ethyl-proyl)-ureido]-phenoxy}-3-methyl-phenyl)-benzamide

### Example 205

The desired product is obtained from N-(4-{4-[3-(1-Ethyl-propyl)-ureido]-phenoxy}-3-methyl-phenyl)-4-(piperidin-4-yloxy)-benzamide and from bromomethylcyclohexane by heating at 90° C. for 24 h, followed by the addition of 2 additional eq of halide and of DIEA and heating at 90° C. 4 h then at AT for 72 h.

### 4-[1-(2-Ethyl-butyl)-piperidin-4-yloxy]-N-(4-{4-[3-(1-ethyl-propyl)-ureido]-phenoxy}-3-methyl-phenyl)-benzamide

### Example 206

The desired product is obtained from N-(4-{4-[3-(1-Ethyl-propyl)-ureido]-phenoxy}-3-methyl-phenyl) (piperidin-4-yloxy)-benzamide and from 1-bromo-2-ethylbutane by heating at 90° C. for 6 h and, after the addition of the additional quantity of halide and DIEA, heating at 90° C. for 28 h.

### N-(4-{4-[3-(1-Ethyl-propyl)-ureido]-phenoxy}-3-methyl-phenyl)-4-[1-(2-methoxy-ethyl)-piperidin-4-yloxy]-benzamide

### Example 207

The desired product is obtained from N-(4-{4-[3-(1-Ethyl-propyl)-ureido]-phenoxy}-3-methyl-phenyl)-4-(piperidin-4-yloxy)-benzamide and from 2-bromoethylmethyl ether by heating at 90° C. for 24 h and, after addition of the additional quantity of halide and DIEA, heating at 90° C. for 4 h and at AT for 72 h.

### N-(4-{4-[3-(1-Ethyl-propyl)-ureido]-phenoxy}-3-methyl-phenyl)-4-[1-(2-hydroxy-ethyl)-piperidin-4-yloxy]-benzamide

### Example 208

The desired product is obtained from N-(4-{4-[3-(1-Ethyl-propyl)-ureido]-phenoxy}-3-methyl-phenyl)-4-(piperidin-4-yloxy)-benzamide and from 2-bromoethanol, following the operating mode described in Example 207.

### Example 209

### N-(4-{4-[3-(1-Ethyl-propyl)-ureido]-2-methoxy-phenoxy}-phenyl)-4-[8-(4-hydroxy-butyl)-8-aza-bicyclo[3.2.1]oct-(3-endo)-yloxy]-benzamide

A solution of 53 mg of compound obtained such as described in Example 195, in a mixture of concentrated HCl (2.3 ml)/MeOH (2.6 ml) is stirred for 15 h at AT. After evaporation in vacuo, the desired product is isolated in TFA salt form, following the operating mode described in Example 1.

### Example 210

### 4-(8-Aza-bicyclo[3.2.1]oct-(3-endo)-yloxy)-N-(4-{4-[3-(1-ethyl-propyl)-ureido]-2-methoxy-phenoxy}-phenyl)-benzamide

200 mg of compound obtained such as described under Preparation 120 are solubilized in DCM, washed with an aqueous Na₂CO₃ solution and the organic layer is dried over MgSO₄ and filtered. A few drops of a HCl/diethyl ether solution are added to the organic layer which is evaporated in vacuo. The desired product is isolated in HCl salt form, after precipitation of the residue in diethyl ether.

### Example 211

### 4-(8-Aza-bicyclo[3.2.1]oct-(3-endo)-yloxy)-N-(4-{4-[3-(1-ethyl-propyl)-ureido]-2-methoxy-phenoxy}-phenyl)-N-(2-methoxy-ethyl)-benzamide

### A/ (3-endo)-(4-{(2-Methoxy-ethyl)-[4-(2-methoxy-4-nitro-phenoxy)-phenyl]-carbamoyl}-phenoxy)-8-aza-bicyclo[3.2.1]octane-8-tertbutyl carboxylate

A mixture of 2.1 g of compound obtained such as described under Preparation 128, step A, 1.01 ml of 2-bromoethylmethyl ether and 5.8 g of Cs₂CO₃ in 15 ml dry DMSO is stirred at AT for 48 h. The reaction medium is diluted with water, the precipitate filtered, dissolved in DCM, and the organic layer is dried over MgSO₄, filtered and evaporated. 1.54 g of desired product are isolated, after chromatography on silica eluting with an ethyl acetate/cyclohexane mixture (40:60 v/v).

### B/ (3-endo)-{4-[[4-(4-Amino-2-methoxy-phenoxy)-phenyl]-(2-methoxy-ethyl)-carbamoyl]-phenoxy}-8-aza-bicyclo[3.2.1]octane-8-tertbutyl carboxylate

1.33 g of desired product are isolated by following General Procedure E to treat the compound obtained in the preceding step.

### C/ (3-endo)-{4-[(4-{4-[3-(1-Ethyl-propyl)-ureido]-2-methoxy-phenoxy}-phenyl)-(2-methoxy-ethyl)-carbamoyl]-phenoxy}-8-aza-bicyclo[3.2.1]octane-8-tertbutyl carboxylate

The compound of the preceding step is treated according to General Procedure H. 1.4 g of desired product are isolated, after chromatography on silica eluting with an ethyl ether/cyclohexane mixture (30:70 v/v).

### D/ 4-(8-Aza-bicyclo[3.2.1]oct-(3-endo)-yloxy)-N-(4-{4-[3-(1-ethyl-propyl)-ureido]-2-methoxy-phenoxy}-phenyl)-N-(2-methoxy-ethyl)-benzamide

The compound of the preceding step is treated following General Procedure C. The desired product is isolated in hydrochloride form, after chromatography on silica eluting with a DCM/MeOH/NH₄OH mixture (90:10:0.1 v/v/v) followed by treatment with a HCl/diethyl ether solution.

### Example 212

### N-(4-{4-[3-(1-Ethyl-propyl)-ureido]-2-methoxy-phenoxy}-phenyl)-4-[1-(3-methoxy-propyl)-piperidin-4-yloxy]-benzamide

85 mg of N-(4-{4-[3-(1-Ethyl-propyl)-ureido]-2-methoxy-phenoxy}-phenyl)-4-(piperidin-4-yloxy)-benzamide are placed in solution in DMF (1.5 ml DMF/0.1 mmol amine), 2.2 eq of DIEA and 1.2 eq of 1-bromo-3-methoxy-propane are added, the reaction medium is heated at 80° C. for 15 h, 0.5 eq of halide and of DIEA are added, heating continued at 80° C. for 15 h and finally a further 0.5 eq of halide and of DIEA are added and heating continued at 80° C. for 15 h. The solvent is evaporated in vacuo, and the desired product is isolated in TFA salt form after purification by semi-preparative HPLC, following the operating mode described in Example 1.

Following the same operating mode as described in Example 212, the following compounds are obtained:

### N-(4-{4-[3-(1-Ethyl-propyl)-ureido]-2-methoxy-phenoxy}-phenyl)-4-[1-(2-hydroxy-ethyl)-piperidin-4-yloxy]-benzamide

### Example 213

The desired product is obtained from N-(4-{4-[3-(1-Ethyl-propyl)-ureido]-2-methoxy-phenoxy}-phenyl)-4-(piperidin-4-yloxy)-benzamide and from 2-bromoethanol, 1 eq of halide and of DIEA still being added during the second heating step.

### N-(4-{4-[3-(1-Ethyl-propyl)-ureido]-2-methoxy-phenoxy}-3-methyl-phenyl)-4-[1-(3-hydroxy-propyl)-piperidin-4-yloxy]-benzamide

### Example 214

The desired product is obtained from N-(4-{4-[3-(1-Ethyl-propyl)-ureido]-2-methoxy-phenoxy}-3-methyl-phenyl)-4-(piperidin-4-yloxy)-benzamide and from 3-bromo-1-propanol, the second heating step being conducted at 90° C. for 24 h, and the third heating step being conducted at 90° C. for 120 h.

### 4-[1-(2-Ethoxy-ethyl)-piperidin-4-yloxy]-N-(4-{4-[3-(1-ethyl-propyl)-ureido]-2-methoxy-phenoxy}-phenyl)-benzamide

### Example 215

The desired product is obtained from N-(4-{4-[3-(1-Ethyl-propyl)-ureido]-2-methoxy-phenoxy}-phenyl)-4-(piperidin-4-yloxy)-benzamide and from 2-bromoethyl ethyl ether.

### N-(4-{4-[3-(1-Ethyl-propyl)-ureido]-2-methoxy-phenoxy}-phenyl)-4-[1-(2-methoxy-ethyl)-piperidin-4-yloxy]-benzamide

### Example 216

The desired product is obtained from N-(4-{4-[3-(1-Ethyl-propyl)-ureido]-2-methoxy-phenoxy}-phenyl)-4-(piperidin-4-yloxy)-benzamide and from 2-bromoethylmethyl ether, 1 eq of halide and of DIEA being added during the second and third heating steps.

### N-(4-{4-[3-(1-Ethyl-propyl)-ureido]-2-methoxy-phenoxy}-3-methyl-phenyl)-4-[1-(3-methyl-butyl)-piperidin-4-yloxy]-benzamide

### Example 217

The desired product is obtained from N-(4-{4-[3-(1-Ethyl-propyl)-ureido]-2-methoxy-phenoxy}-3-methyl-phenyl)-4-(piperidin-4-yloxy)-benzamide and from 1-bromo-3-methylbutane, following the operating mode described in Example 214.

### 4-(1-Butyl-piperidin-4-yloxy)-N-(4-{4-[3-(1-ethyl-propyl)-ureido]-2-methoxy-phenoxy}-phenyl)-N-isobutyl-benzamide

### Example 218

The desired product is obtained from N-(4-{4-[3-(1-Ethyl-propyl)-ureido]-2-methoxy-phenoxy}-phenyl)-N-isobutyl-4-(piperidin-4-yloxy)-benzamide and 1-bromobutane, 1 eq of halide and of DEIA being added during the second and the third heating steps.

### 4-(1-sec-Butyl-piperidin-4-yloxy)-N-(4-{4-[3-(1-ethyl-propyl)-ureido]-phenoxy}-3-methyl-phenyl)-benzamide

### Example 219

The desired product is obtained from N-(4-{4-[3-(1-Ethyl-propyl)-ureido]-phenoxy}-3-methyl-phenyl)-4-(piperidin-4-yloxy)-benzamide and 2-bromobutane, 1.5 eq and respectively 1 eq of halide and DIEA being added during the second and third heating steps.

### N-(4-{4-[3-(1-Ethyl-propyl)-ureido]-2-methoxy-phenoxy}-phenyl)-4-[1-(3,3,3-trifluoro-propyl)-piperidin-4-yloxy]-benzamide

### Example 220

The desired product is obtained from N-(4-{4-[3-(1-Ethyl-propyl)-ureido]-2-methoxy-phenoxy}-phenyl)-piperidin-4-yloxy)-benzamide and 1-bromo-3,3,3-trifluoropropane, following the operating mode described in Example 216.

### Example 221

### N-(4-{4-[3-(1-Ethyl-propyl)-ureido]-2-methoxy-phenoxy}-phenyl)-4-[1-(3-hydroxy-propyl)-piperidin-4-yloxy]-benzamide

500 mg of N-(4-{4-[3-(1-Ethyl-propyl)-ureido]-2-methoxy-phenoxy}-phenyl)-4-(piperidin-4-yloxy)-benzamide, 107 μL of 3-bromo-1-propanol (1.5 eq) and 570 mg of K₂CO₃ (5 eq) are placed in suspension in 4 ml DMF, stirred 5 h at 75° C., an additional 3 eq of 3-bromo-1-propanol are added and heated 10 h at 75° C. The solvent is evaporated, the residue redissolved in water and the precipitate obtained washed with pentane. The desired product is isolated in TFA salt form, after chromatography on silica eluting with a DCM/MeOH/NH₄OH mixture (92.5:7,5:0.5 v/v/v) followed by semi-preparative HPLC in accordance with the operating mode described in Example 1.

Following the same operating mode as described in Example 221, the following compounds are obtained:
N-(4-{4-[3-(1-Ethyl-propyl)-ureido]-2-methoxy-phenoxy}-phenyl)-4-[1-(3-methyl-butyl)-piperidin-4-yloxy]-benzamide

### Example 222

The desired product is obtained from N-(4-{4-[3-(1-Ethyl-propyl)-ureido]-2-methoxy-phenoxy}-phenyl)-4-(piperidin-4-yloxy)-benzamide and from 1-bromo-3-methylbutane. The desired product is isolated after chromatography on silica eluting with a DCM/MeOH/NH₄OH mixture (92.5:7.5:0.5 v/v/v). The hydrochloride is obtained by treating with a HCl/diethyl ether mixture.

### 4-[1-(2-Dimethylamino-ethyl)-piperidin-4-yloxy]-N-(4-{4-[3-(1-ethyl-propyl)-ureido]-2-methoxy-phenoxy}-phenyl)-benzamide

### Example 223

Other desired product is obtained from N-(4-{4-[3-(1-Ethyl-propyl)-ureido]-2-methoxy-phenoxy}-phenyl)-4-(piperidin-4-yloxy)-benzamide and from (2-chloro-ethyl)-dimethyl-amine. The desired product is isolated after chromatography on silica eluting with a DCM/MeOH/NH₄OH mixture (90:10:0.1 v/v/v). The hydrochloride is obtained by treatment with a HCl/diethyl ether mixture.

### 4-(1-Butyl-piperidin-4-yloxy)-N-(4-{4-[3-(1-ethyl-propyl)-ureido]-2-methoxymethyl-phenoxy}-phenyl)-benzamide

### Example 224

The desired product is obtained from N-(4-{4-[3-(1-Ethyl-propyl)-ureido]-2-methoxymethyl-phenoxy}-phenyl)-4-(piperidin-4-yloxy)-benzamide and from 1-bromobutane in the presence of 1.5 additional eq of halide. The desired product is isolated after chromatography on silica eluting with a DCM/MeOH/NH₄OH mixture (95:5:0.5 v/v/v). The hydrochloride is obtained by treating with a HCl/diethyl ether mixture.

### Example 225

### N-(4-{4-[3-(1-Ethyl-propyl)-ureido]-2-methoxy-phenoxy}-phenyl)-4-[1-(1-methyl-butyl)-piperidin-4-yloxy]-benzamide

100 mg of N-(4-{4-[3-(1-Ethyl-propyl)-ureido]-2-methoxy-phenoxy}-phenyl)-4-(piperidin-4-yloxy)-benzamide, 29 μL of DIEA (1 eq), 120 mg of Na₂SO₄ (0.5 eq) and 35 μL of 2-pentanone (2 eq) are placed in suspension in 1 ml of an ACN/chloroform mixture (1:1 v/v), stirred 24 h at AT, 19 mg of NaBH₄ (3 eq) are added, heated under reflux for 6 h and at AT for 72 h, 1 eq of 2-pentanone and 3 eq of NaBH₄ are added and heated under reflux 72 h. The solvent is evaporated, the residue redissolved in DMF, the salts filtered, followed by semi-preparative HPLC purification. The desired product is isolated in TFA salt form, following the operating mode described in Example 1.

Following the same operating mode as described in Example 225, the following compounds are obtained:

### N-(4-{4-[3-(1-Ethyl-propyl)-ureido]-2-methoxy-phenoxy}-phenyl)-4-[1-(Tetrahydro-pyran-4-yl)-piperidin-4-yloxy]-benzamide

### Example 226

The desired product is obtained from N-(4-{4-[3-(1-Ethyl-propyl)-ureido]-2-methoxy-phenoxy}-phenyl)-4-(piperidin-4-yloxy)-benzamide and Tetrahydro-4H-pyran-4-one.

### N-(4-{4-[3-(1-Ethyl-propyl)-ureido]-phenoxy}-3-methyl-phenyl)-4-[1-(1-hydroxymethyl-propyl)-piperidin-4-yloxy]-benzamide

### Example 227

The desired product is obtained from N-(4-{4-[3-(1-Ethyl-propyl)-ureido]-phenoxy}-3-methyl-phenyl)-4-(piperidin-4-yloxy)-benzamide and from 1-hydroxy-2-butanone in the presence of 2 additional eq of ketone. The desired product is isolated in free base form after purification of the reaction medium following the SPE technique on 2 g SCX cartridge, leaving a deposit in 3 mL of DMF, washing with 10 ml MeOH and elution with a 2M ammonia solution in MeOH.

### 4-(1-Cyclobutyl-piperidin-4-yloxy)-N-(4-{4-[3-(1-ethyl-propyl)-ureido]-phenoxy}-3-methyl-phenyl)-benzamide

### Example 228

The desired product is obtained from N-(4-{4-[3-(1-Ethyl-propyl)-ureido]-phenoxy}-3-methyl-phenyl)-4-(piperidin-4-yloxy)-benzamide and cyclobutanone, in the presence of an additional 2 eq of ketone.

### N-(4-{4-[3-(1-Ethyl-propyl)-ureido]-phenoxy}-3-methyl-phenyl)-4-[1-(1-methyl-butyl)-piperidin-4-yloxy]-benzamide

### Example 229

The desired product is obtained from N-(4-{4-[3-(1-Ethyl-propyl)-ureido]-phenoxy}-3-methyl-phenyl)-4-(piperidin-4-yloxy)-benzamide and 2-pentanone, in the presence of an additional 2 eq of ketone.

### 4-(1-Cyclopentyl-piperidin-4-yloxy)-N-(4-{4-[3-(1-ethyl-propyl)-ureido]-phenoxy}-3-methyl-phenyl)-benzamide

### Example 230

The desired product is obtained from N-(4-{4-[3-(1-Ethyl-propyl)-ureido]-phenoxy}-3-methyl-phenyl)-4-(piperidin-4-yloxy)-benzamide and from cyclopentanone, by heating under reflux for 9 h, with no additional adding of ketone or reducer.

### Example 231

### N-(4-{4-[3-(1-Ethyl-propyl)-ureido]-phenoxy}-3-methyl-phenyl)-4-(1-propyl-piperidin-4-yloxy)-benzamide

148 mg of N-(4-{4-[3-(1-Ethyl-propyl)-ureido]-phenoxy}-3-methyl-phenyl)-4-(piperidin-4-yloxy)-benzamide and 14.4 μL of propionaldehyde are placed in suspension in 2 ml chloroform, heated under reflux for 1 h, followed by the addition of 150 mg of sodium triacetoxyborohydride, heating under reflux 72 h, evaporation of the solvent, redissolving the residue in DMF and filtering the salts. The desired product is isolated in TFA salt form after purification by semi-preparative HPLC, following the operating mode described in Example 1.

Following the same operating mode as in Example 131, the following compounds are obtained:

### 4-(1-Butyl-piperidin-4-yloxy)-N-(4-{4-[3-(1-ethyl-propyl)-ureido]-phenoxy}-3-methyl-phenyl)-benzamide

### Example 232

The desired product is obtained from N-(4-{4-[3-(1-Ethyl-propyl)-ureido]-phenoxy}-3-methyl-phenyl)-4-(piperidin-4-yloxy)-benzamide and from butyraldehyde.

### N-(4-{4-[3-(1-Ethyl-propyl)-ureido]-phenoxy}-3-methyl-phenyl)-4-(1-methyl-piperidin-4-yloxy)-benzamide

### Example 233

The desired product is obtained from N-(4-{4-[3-(1-Ethyl-propyl)-ureido]-phenoxy}-3-methyl-phenyl)-4-(piperidin-4-yloxy)-benzamide and from formaldehyde in a 37% aqueous solution.

### N-(4-{4-[3-(1-Ethyl-propyl)-ureido]-phenoxy}-3-methyl-phenyl)-4-[1-(3-methyl-butyl)-piperidin-4-yloxy]-benzamide

### Example 234

The desired product is obtained from N-(4-{4-[3-(1-Ethyl-propyl)-ureido]-phenoxy}-3-methyl-phenyl)-4-(piperidin-4-yloxy)-benzamide and from isovaleraldehyde.

### 4-(1-Ethyl-piperidin-4-yloxy)-N-(4-{4-[3-(1-ethyl-propyl)-ureido]-phenoxy}-3-methyl-phenyl)-benzamide

### Example 235

The desired product is obtained from N-(4-{4-[3-(1-Ethyl-propyl)-ureido]-phenoxy}-3-methyl-phenyl)-1-piperidin-4-yloxy)-benzamide and from acetaldehyde, in the presence of 1 additional eq of aldehyde and 3 additional eq of reducer, and refluxed for 48 h.

### N-(4-{4-[3-(1-Ethyl-propyl)-ureido]-phenoxy}-3-methyl-phenyl)-4-(1-isobutyl-piperidin-4-yloxy)-benzamide

### Example 236

The desired product is obtained from N-(4-{4-[3-(1-Ethyl-propyl)-ureido]-phenoxy}-3-methyl-phenyl)-4-(piperidin-4-yloxy)-benzamide and from isobutyraldehyde.

### 4-(1-Cyclopropyl-piperidin-4-yloxy)-N-(4-{4-[3-(1-ethyl-propyl)-ureido]-phenoxy}-3-methyl-phenyl)-benzamide

### Example 237

The desired product is obtained from N-(4-{4-[3-(1-Ethyl-propyl)-ureido]-phenoxy}-3-methyl-phenyl)-4-(piperidin-4-yloxy)-benzamide and from [(1-ethoxycyclopropyl)oxy]trimethylsilane (4 eq) in the presence of 6 eq of reducer and 1 eq of acetic acid, by heating 6 h under reflux and 72 h at AT. The desired product is isolated in free base form after purifying the reaction medium following the SPE technique on SCX cartridge, as per the operating mode described in Example 227.

### Example 238

### 4-(1-Butyl-piperidin-4-yloxy)-N-(4-{4-[3-(1-ethyl-propyl)-ureido]-2-methoxy-phenoxy}-2-fluoro-phenyl)-benzamide

Following General Procedure H, 750 mg of N-[4-(4-Amino-2-methoxy-phenoxy)-2-fluoro-phenyl]1-butyl-piperidin-4-yloxy)-benzamide and 540 mg of imidazole-1-carboxylic acid (1-ethyl-propyl)-amide are heated under reflux in THF for 24 h, then an additional 4 eq of imidazole-1-carboxylic acid (1-ethyl-propyl)-amide are added and heated under reflux for 120 h. The solvent is evaporated in vacuo, the residue redissolved in DCM, the organic layer is washed with water, dried over MgSO₄, filtered and evaporated. The desired product is isolated in TFA salt form after semi-preparative HPLC purification, following the protocol described in Example 1.

Following the same operating mode as described in Example 238, the following compounds are obtained:

### N-(4-{4-[3-(1-Ethylpropyl)-ureido]-2-methoxy-phenoxy}-phenyl)-4-(8-methyl-8-aza-bicyclo[3.2.1]oct-(3-endo)-yloxy)-N-propyl-benzamide

### Example 239

The desired product is obtained from N-[4-(4-Amino-2-methoxy-phenoxy)-phenyl]4-(8-methyl-8-aza-bicyclo[3.2.1]oct-(3-endo)-yloxy)-N-propyl-benzamide, after adding only 2 additional eq of imidazole-1-carboxylic acid (1-ethyl-propyl)-amide then heating under reflux for 48 h. The desired product is isolated in free base form after purification by semi-preparative HPLC, followed by chromatography on silica eluting with a DCM/MeOH/NH₄OH mixture (90:10:0.1 v/v/v).

### N-(4-{4-[3-(1-Ethyl-propyl)-ureido]-2-methoxy-phenoxy}-2-fluoro-phenyl)-4-(8-methyl-8-aza-bicyclo[3.2.1]oct-(3-endo)-yloxy)-benzamide

### Example 240

The desired product is obtained from N-[4-(4-Amino-2-methoxy-phenoxy)-2-fluoro-phenyl]-4-(8-methyl-8-aza-bicyclo[3.2.1]oct-(3-endo)-yloxy)-benzamide, following the operating mode described in Example 239. The desired product is isolated in TFA salt form after purification by semi-preparative HPLC, following the protocol described in Example 1.

### 4-(1-Butyl-piperidin-4-yloxy)-N-(4-{4-[3-(1-ethyl-propyl)-ureido]-2-methoxy-phenoxy}-2-fluoro-phenyl)-3-methyl-benzamide

### Example 241

The desired product is obtained from N-[4-(Amino-2-methoxy-phenoxy)-2-fluoro-phenyl]-4-(1-butyl-piperidin-4-yloxy)-3-methyl-benzamide following the operating mode described in Example 239, in the presence of 1 eq of DIEA and a drop of pyridine. The desired product is isolated in hydrochloride form after purification by semi-preparative HPLC, following the protocol described in Example 19.

### 4-(1-Butyl-piperidin-4-yloxy)-N-{8-[3-(1-ethyl-propyl)-ureido]-10,11-dihydro-dibenzo[b,f]oxepin-2-yl}-benzamide

### Example 242

The desired product is obtained from N-(8-Amino-10,11-dihydro-dibenzo[b,f]oxepin-2-yl)-4-(1-butyl-piperidin-4-yloxy)-benzamide in the presence of 2 eq of imidazole-1-carboxylic acid (1-ethyl-propyl)-amide and by heating under reflux for 24 h. The desired product is obtained in free base form after chromatography on silica eluting with a DCM/MeOH/NH₄OH mixture (90:10:0.1 v/v/v). The hydrochloride is isolated after treating with a HCl/diethyl ether mixture.

### N-(4-{4-[3-(1-Ethyl-propyl)-ureido]-2-methoxy-phenoxy}-phenyl)-N-methyl-4-(8-methyl-aza-bicyclo[3.2.1]oct-(3-endo)-yloxy)-benzamide

### Example 243

The desired product is obtained from N-[4-(4-Amino-2-methoxy-phenoxy)-phenyl]-N-methyl-4-(8-methyl-8-aza-bicyclo[3.2.1]oct-(3-endo)-yloxy)-benzamide in the presence of 2 eq imidazole-1-carboxylic acid (1-ethyl-propyl)-amide, 1 eq of DIEA and a drop of pyridine and heating under reflux for 72 h. The desired product is isolated in hydrochloride form following the operating mode described in Example 242.

### N-[4-(1-Butyl-piperidin-4-yloxy)-phenyl]-4-{4-[3-(1-ethyl-propyl)-ureido]-2-methoxy-phenoxy}-benzamide

### Example 244

The desired product is obtained from 4-(4-Amino-2-methoxy-phenoxy)-N-[4 (1-butyl-piperidin-4-yloxy)-phenyl]-benzamide in the presence of 3 eq of imidazole-1-carboxylic acid (1-ethyl-propyl)-amide and by heating under reflux for 72 h. The desired product is isolated in TFA salt form, after semi-preparative HPLC purification, following the procedure described in Example 1.

### Example 245

### 1-(4-{2-[4-(1-Butyl-piperidin-4-yloxy)-phenyl]-1-methyl-1H-benzoimidazol-5-yloxy}-3-methoxy-phenyl)-3-(1-ethyl-propyl)-urea

### A/ (4-Methoxy-2-nitro-phenyl)-methyl-amine

10 g of 4-methoxy-2-nitroaniline are added to a suspension of NaH (1.3 eq) in DMF in an inert atmosphere at 0° C., stirred for 1 h at 0° C., followed by the addition of 1.1 eq of methyl iodide and stirring for 2 h at 0° C. and 2 h at AT. After evaporation in vacuo, the residue is redissolved in water, extracted with TBME, the organic layer is washed with water, dried over MgSO₄, filtered and evaporated. 10.4 g of desired product are obtained in the form of an orange solid.

### B/ 4-Methoxy-N*1*-methyl-benzene-1,2-diamine

Following General Procedure E, 5.5 g of desired product are obtained from the compound of the preceding step.

### C/ 4-(1-Butyl-piperidin-4-yloxy)-N-(5-methoxy-2-methylamino-phenyl)-benzamide

Following General Procedure L1, the compound obtained in the preceding step is reacted with 4-(1-Butyl-piperidin-4-yloxy)-benzoic acid (Preparation 5). On completion of the reaction, the solvent is evaporated in vacuo, the residue redissolved in water, extracted with ethyl acetate, and the organic layer is washed with an aqueous NaHCO3 solution and with water, dried over MgSO₄, filtered and evaporated. 1.6 g of desired product are obtained in the form of a pink powder, after precipitation of the residue in diethyl ether.

### D/ 2-[4-(1-Butyl-piperidin-4-yloxy)-phenyl]-5-methoxy-1-methyl-1H-benzoimidazole

4.05 g of compound obtained such as described in the preceding step are heated under reflux for 1 h in a mixture of concentrated HCl (165 ml)/water (82 ml). After concentration in vacuo, redissolving in water, extracting with ethyl acetate, the organic layer is washed with water, dried over MgSO₄, filtered and evaporated. 3 g of desired product are obtained in the form of a brown solid.

### E/ 2-[4-(1-Butyl-piperidin-4-yloxy)-phenyl]-1-methyl-1H-benzoimidazol-5-ol

The product of the preceding step, in solution in 76 ml of 48% HBr, is heated at 135° C. for 1 h. After evaporation in vacuo, the residue is redissolved in water, basified with an aqueous NaHCO₃ solution, extracted with ethyl acetate and the organic layer evaporated. 2.7 g of desired product are obtained in the form of a brown solid.

### F/ 2-[4-(1-Butyl-piperidin-4-yloxy)-phenyl]-5-(2-methoxy-4-nitro-phenoxy)-1-methyl-1H-benzoimidazole

A solution of 2.2 g of compound obtained in the preceding step in 40 ml DMF is added dropwise to a suspension of NaH (1.5 eq) in 90 ml DMF, stirred 1.5 h at 35° C., followed by the addition of 2-chloro-5-nitro-anisole (1.5 eq), heating for 24 h at 80° C. and concentration in vacuo. The residue is redissolved in water, extracted with ethyl acetate, washed with an aqeuous NaCl solution, and the organic layer is dried over MgSO₄, filtered and evaporated. 1.5 g of desired product are obtained in oil form, after chromatography on silica eluting with a DCM/MeOH mixture (90:10 v/v).

### G/ 4-{2-[4-(1-Butyl-piperidin-4-yloxy)-phenyl]-1-methyl-1H-benzoimidazol-5-yloxy}-3-methoxy-phenylamine

The compound of the preceding step, in solution in 100 ml MeOH, in an inert atmosphere and in the presence of 1.2 g of 5% palladium on charcoal is reacted with ammonium formiate (11.6 eq), for 15 h at AT. The catalyst is filtered and rinsed with MeOH, the filtrate concentrated in vacuo, the residue redissolved in DCM, and the organic layer is washed with water, dried over MgSO₄, filtered and evaporated. 0.98 g of desired product are obtained.

### H/ 1-(4-{2-[4-(1-Butyl-piperidin-4-yloxy)-phenyl]-1-methyl-1H-benzoimidazol-5-yloxy}-3-methoxy-phenyl)-3-(1-ethyl-propyl)-urea

The compound of the preceding step is treated following General Procedure H. The desired product is isolated in hydrochloride form after purification by semi-preparative HPLC following the operating mode described in Example 19.

### Example 246

### 1-(4-{2-[4-(1-Butyl-piperidin-4-yloxy)-phenyl]-1-propyl-1H-benzoimidazol-5-yloxy}-3-methoxy-phenyl)-3-(1-ethyl-propyl)-urea

### A/ (4-Methoxy-2-nitro-phenyl)-propyl-amine

5 g of 4-methoxy-2-nitroaniline are added to a suspension of NaH (1.3 eq) in 150 ml DMF in an inert atmosphere at 0° C., stirred for 1 h at 0° C., followed by the addition of 1.1 eq of 1-bromopropane and stirring for 15 h at AT. After evaporation in vacuo, the residue is redissolved in water, extracted with DCM and the organic layer is washed with a saturated aqueous NaCl solution, dried over MgSO₄, filtered and evaporated. 6.2 g of desired product are obtained after chromatography on silica eluting with a cyclohexane/ethyl acetate mixture (95:5 v/v).

### B/ 4-Methoxy-N*1*-propyl-benzene-1,2-diamine

Following General Procedure E, 3.08 g of desired product are obtained from the compound of the preceding step.

### C/ 4-(1-Butyl-piperidin-4-yloxy)-N-(5-methoxy-2-propylamino-phenyl)-benzamide

Following General Procedure L1, the compound obtained in the preceding step is reacted with 4.7 g of 4-(1-Butyl-piperidin-4-yloxy)-benzoic acid (Preparation 5). On completion of the reaction the solvent is evaporated in vacuo, the residue redissolved in water, extracted with DCM and the organic layer is dried over MgSO₄, filtered and evaporated. 4.28 g of desired product are obtained after chromatography on silica eluting with a DCM/MeOH/NH₄OH mixture (95:5:0.05 v/v/v).

### D/ 2-[4-(1-Butyl-piperidin-4-yloxy)-phenyl]-5-methoxy-1-propyl-1H-benzoimidazole

2.84 g of compound obtained such as described in the preceding step are heated under reflux for 1 h in a mixture of concentrated HCl (65 ml)/water. (25 ml). The reaction medium is basified with an aqueous NaHCO₃ solution, extracted with ethyl acetate, the organic layer is washed with water, dried over MgSO₄, filtered and evaporated. 1.25 g of desired product are obtained after chromatography on silica eluting with a DCM/MeOH/NH₄OH mixture (95:5:0.05 v/v/v).

### E/ 2-[4-(1-Butyl-piperidin-4-yloxy)-phenyl]-1-propyl-1H-benzoimidazol-5-ol

The product of the preceding step, in solution in 30 ml of 48% HBr, is heated at 135° C. for 1.5 h. After evaporation in vacuo, the residue is redissolved in water, basified with an aqueous NaHCO₃ solution, extracted with ethyl acetate, and the organic layer evaporated. 1.2 g of desired product are obtained, which is used as such.

### F/ 2-[4-(1-Butyl-piperidin-4-yloxy)-phenyl]-5-(2-methoxy-4-nitro-phenoxy)-1-propyl-1H-benzoimidazole

To a suspension of NaH (1.3 eq) in 10 ml DMF is added the compound obtained in the preceding step, which is stirred 1 h at AT, followed by the addition of 2-chloro-5-nitro-anisole (1 eq), heating for 60 h at 70° C., then concentration in vacuo. The residue is redissolved in water, extracted with TBME, the organic layer is dried over MgSO₄, filtered and evaporated. 0.74 g of desired product are obtained in oil form after chromatography on silica eluting with a DCM/MeOH/NH₄OH mixture (95:5:0.05 v/v/v).

### G/ 4-{2-[4-(1-Butyl-piperidin-4-yloxy)-phenyl]-1-propyl-1H-benzoimidazol-5-yloxy}-3-methoxy-phenylamine

0.7 g of desired product are obtained from the compound of the preceding step following General Procedure E.

### H/ 1-(4-{2-[4-(1-Butyl-piperidin-4-yloxy)-phenyl]-1-propyl-1H-benzoimidazol-5-yloxy}-3-methoxy-phenyl)-3-(1-ethyl-propyl)-urea

The compound of the preceding step is treated following General Procedure H. The desired product is isolated in free base form after chromatography on silica eluting with a DCM/MeOH/NH₄OH mixture (95:5:0.05 v/v/v). The hydrochloride is obtained by treating with a HCl/diethyl ether solution.

### Example 247

### 1-(4-{2-[4-(1-Butyl-piperidin-4-yloxy)-phenyl]-1-ethyl-1H-indol-5-yloxy}-3-methoxy-phenyl)-3-(1-ethyl-propyl)-urea

### A/ 4-(1-Butyl-piperidin-4-yloxy)-N-(4-methoxy-2-methyl-phenyl)-benzamide

Following General Procedure L1, 10 g of 4-(1-Butyl-piperidin-4-yloxy)-benzoic acid (Preparation 5) are reacted with 4-methoxy-2-methylaniline (1 eq). On completion of the reaction, the medium is evaporated in vacuo, the residue redissolved in an aqueous NaOH solution, extracted with DCM and the organic layer is dried over MgSO₄, filtered and evaporated. 9.2 g of desired product are obtained.

### B/ 2-[4-(1-Butyl-piperidin-4-yloxy)-phenyl]-5-methoxy-1H-indole

The compound of the preceding step is placed in solution in 80 ml anhydrous THF, and 28 ml of a 2.5 M nBuLi solution in THF are added dropwise at 0° C., and stirred 1 h at AT. Then an aqueous ammonium chloride solution is added dropwise, followed by dilution with water, extraction with TBME, the organic layer is dried over MgSO₄, filtered, evaporated and the crystals obtained are washed with TBME. 5.8 g of desired product are isolated.

### C/ 2-[4-(1-Butyl-piperidin-4-yloxy)-phenyl]-1-ethyl-5-methoxy-1H-indole

To a suspension of 0.73 g of NaH in 50 ml DMF is added the compound of the preceding step, the mixture stirred 1 h at AT, 1.6 ml of ethyl iodide are added and stirred 15 h at AT. After evaporation in vacuo, the residue is redissolved in water, extracted with DCM, the organic layer dried over MgSO₄, filtered and evaporated. 5.2 g of desired product are obtained after chromatography on silica eluting with a DCM/MeOH mixture (95:5 v/v).

### D/ 2-[4-(1-Butyl-piperidin-4-yloxy)-phenyl]-1-ethyl-1H-indol-5-ol

The product of the preceding step, in solution in 300 ml of 48% HBr, is heated under reflux for 1.5 h. After evaporation in vacuo, the residue is redissolved in water, basified with an aqueous NaHCO3 solution, extracted with DCM and the organic layer is dried over MgSO₄, filtered and evaporated. 3.5 g of desired product are obtained after chromatography on silica eluting with a DCM/MeOH mixture (95:5 v/v).

### E/ 2-[4-(1-Butyl-piperidin-4-yloxy)-phenyl]-1-ethyl-5-(2-methoxy-4-nitro-phenoxy)-1H-indole

To a suspension of 0.5 g of NaH (1.3 eq) in DMF is added the compound obtained in the preceding step, and stirred 1 h at AT, 1.7 g of 2-chloro-5-nitro-anisole are added, followed by heating for 6 h at 60° C. and concentration in vacuo. The residue is redissolved in water, extracted with DCM, the organic layer is dried over MgSO₄, filtered and evaporated. 3.3 g of desired product are obtained after chromatography on silica, eluting with a DCM/MeOH mixture (97.5:2.5 v/v).

### F/ 4-{2-[4-(1-Butyl-piperidin-4-yloxy)-phenyl]-1-ethyl-1H-indol-5-yloxy}-3-methoxy-phenylamine

2.9 g of desired product are obtained from the compound of the preceding step, following General Procedure E.

### G/ 1-(4-{2-[4-(1-Butyl-piperidin-4-yloxy)-phenyl]-1-ethyl-1H-indol-5-yloxy}-3-methoxy-phenyl)-3-(1-ethyl-propyl)-urea

The compound of the preceding step is treated following General Procedure H. The desired product is isolated in hydrochloride form after purification by semi-preparative HPLC, following the operating mode described in Example 19.

### Example 248

### 1-(4-{4-[6-(1-Butyl-piperidin-4-yloxy)-benzooxazol-2-yl]-phenoxy}-3-methoxy-phenyl)-3-(1-ethyl-propyl)-urea

### A/ 4-Methoxy-2-(4-methoxy-phenyl)-benzooxazol-6-yl benzoate

A mixture of 100 g of p-anisoyl chloride and 17.8 g of 4-aminoresorcinol is heated at 200° C. for 2 h, cooled to AT, an aqueous NaHCO₃ solution is added and stirred at AT for 15 h. After extraction with DCM, the organic layer is dried over MgSO₄, filtered and evaporated. 68 g of desired product are obtained, which is used as such.

### B/ 2-(4-Methoxy-phenyl)-benzooxazol-6-ol

The compound of the preceding step, in suspension in water, is heated under reflux for 2 h in the presence of 15 g of NaOH. The reaction medium is concentrated, acidified to pH 4, then neutralized with an aqueous NaHCO₃ solution, extracted with DCM, the organic layer is dried over MgSO₄, filtered and evaporated. 11 g of desired product are obtained after chromatography on silica eluting with a DCM/MeOH mixture (97:3 v/v).

### C/ 6-(1-Butyl-piperidin-4-yloxy)-2-(4-methoxy-phenyl)-benzooxazole

To a solution of 15.7 g of triphenylphosphine in THF (300 ml), cooled to −15° C., are added dropwise 12.1 g of DIAD in THF (100 ml), stirred 15 min at −15° C., then a mixture of 14.4 g of compound obtained such as described in the preceding step and 9.4 g of 1-butyl-piperidin-4-ol (Preparation 3, step A) in THF (300 ml) is added dropwise and stirred 48 h at AT. After evaporation in vacuo, an aqueous HCl solution is added, followed by washing with TBME, the aqueous layer is basified and extracted with TBME, this last organic layer is dried over MgSO₄, filtered and evaporated. 4.1 g of desired product are obtained after chromatography on silica eluting with a DCM/MeOH mixture (99.5:0.5 v/v).

### D/ 4-[6-(1-Butyl-piperidin-4-yloxy)-benzooxazol-2-yl]-phenol

3.6 g of compound obtained in the preceding step are heated at 170° C. for 4 h in the presence of 70 g of pyridine hydrochloride. An aqueous NaHCO₃ solution is added, the precipitate obtained is filtered, washed with water, dissolved in DCM and the organic layer is washed with an aqueous ammonia solution, dried over MgSO₄, filtered and evaporated. 2.2 g of desired product are obtained.

### E/ 6-(1-Butyl-piperidin-4-yloxy)-2-[4-(2-methoxy-4-nitro-phenoxy)-phenyl]-benzooxazole

To a suspension of 0.5 g of NaH in DMF is added the compound obtained in the preceding step, stirred 1 h at AT, followed by the addition of 1.2 g of 2-chloro-5-nitro-anisole, heating for 50 h at 60° C. and concentration in vacuo. The residue is redissolved in water, extracted with TBME, the organic layer is dried over MgSO₄, filtered and evaporated. 0.24 g of desired product are obtained after chromatography on silica eluting with a DCM/MeOH mixture (85:15 v/v).

### F/ 4-{4-[6-(1-Butyl-piperidin-4-yloxy)-benzooxazol-2-yl]-phenoxy}-3-methoxy-phenylamine

0.2 g of desired product are obtained from the compound of the preceding step, following General Procedure E.

### G/ 1-(4-{4-[6-(1-Butyl-piperidin-4-yloxy)-benzooxazol-2-yl]-phenoxy}-3-methoxy-phenyl)-3-(1-ethyl-propyl)-urea

The compound of the preceding step is treated following General Procedure H. The desired product is isolated in hydrochloride form, after purifying by semi-preparative HPLC, following the operating mode described in Example 19.

### Example 249

### 1-(4-{4-[6-(1-Butyl-piperidin-4-yloxy)-benzooxazol-2-yl]-phenoxy}-phenyl)-3-(1-ethyl-propyl)-urea

### A/ 6-(1-Butyl-piperidin-4-yloxy)-2-[4-(4-nitro-phenoxy)-phenyl]-benzooxazole

To a suspension of 0.3 g of NaH in 10 ml DMF are added 1.1 g of compound obtained such as described in Example 248, step D, the mixture stirred 1 h at AT, 0.8 g of 4-fluoronitrobenzene are added and stirring continued 50 h at AT followed by concentration in vacuo. The residue is redissolved in water, extracted with DCM, the organic layer dried over MgSO₄, filtered and evaporated. 1.2 g of desired product are obtained after crystallization in TBME.

### B/ 4-{4-[6-(1-Butyl-piperidin-4-yloxy)-benzooxazol-2-yl]-phenoxy}-phenylamine

1 g of product is obtained from the compound of the preceding step, following General Procedure E.

### C/ 1-(4-{4-[6-(1-Butyl-piperidin-4-yloxy)-benzooxazol-2-yl]-phenoxy}-phenyl)-3-(1-ethyl-propyl)-urea

The compound of the preceding step is treated following General Procedure H. The desired product is isolated in hydrochloride form, after purification by semi-preparative HPLC, following the operating mode described in Example 19.

### Example 250

### 1-(1-Ethyl-propyl)-3-(3-methoxy-4-{4-[5-(1-methyl-piperidin-4-yloxy benzofuran-2-yl]-phenoxy}-phenyl)-urea

### A/ Benzofuran-5-ol

19.5 g of 5-methoxybenzofurane are heated at 170° C. for 3 h, in the presence of 66 g of pyridine hydrochloride. Water is added, extraction made with TBME, the organic layer is extracted with an aqueous NaOH solution, this aqueous layer is acidified and extracted with TBME. The last TBME layer is dried over MgSO₄, filtered and evaporated. 14.3 g of desired product are obtained.

### B/ (Benzofuran-5-yloxy)-tert-butyl-dimethyl-silane

To a mixture of the compound of the preceding step, of 24.3 g of imidazole and of 0.1 g DMAP in 40 ml DMF, are added dropwise, keeping the temperature to below 25° C., 28 g of tertbutyldimethylsilyl chloride and stirred 48 h at AT. The reaction medium is diluted with water, extracted with TBME, the organic layer is washed with an aqueous NaOH solution and with water, dried over MgSO₄, filtered and evaporated. 22 g of desired product are obtained after chromatography on silica eluting with pentane.

### C/ tert-Butyl-[2-(4-methoxy-phenyl)-benzofuran-5-yloxy]-dimethyl-silane

To a solution of 21.8 g of compound of the preceding step in 85 ml THF, are added dropwise at −10° C., 35.1 ml of 2.5 M BuLi solution in THF and stirred 2 h at −10° C., then a solution of 29.9 g of ZnBr₂ in 450 ml THF is added dropwise, stirred 1 h at AT, followed by the addition of 20.5 g of 4-iodoanisole in 60 ml THF and 4.4 g of Tetrakis triphenylphosphine palladium and stirring for 48 h at AT. While keeping the temperature to −10° C., an aqueous ammonium chloride solution and then water are added dropwise, extraction made with TBME, then drying over MgSO₄, filtering and evaporation. 18.7 g of desired product are obtained.

### D/ 2-(4-Methoxy-phenyl)-benzofuran-5-ol

The compound of the preceding step is solubilized in 180 ml THF, followed by the addition of 160 ml of a 1 M TBAF solution in THF and stirring for 3 h at AT. After diluting with water, extracting with TBME, drying is performed over MgSO₄, followed by filtration and evaporation. 6.4 g of desired product are obtained after crystallization in DCM.

### E/ 4-[2-(4-Methoxy-phenyl)-benzofuran-5-yloxy]-1-methyl-piperidine

To a solution of 14 g of triphenylphosphine in THF (300 ml), cooled to −15° C., are added dropwise 10.9 g of DIAD in THF (100 ml), stirred 15 min at −15° C., followed by the dropwise addition of a mixture of the compound obtained such as described in the preceding step and of 6.1 g of 1-methyl piperidinol in THF (300 ml), and stirring continued for 48 h at AT. After evaporation in vacuo, an aqueous NaOH solution is added, extraction made with TBME, the organic layer is dried over MgSO₄, filtered and evaporated. 6 g of desired product are obtained after chromatography on silica eluting with a DCM/MeOH mixture (90:10 v/v).

### F/ 4-[5-(1-Methyl-piperidin-4-yloxy)-benzofuran-2-yl]-phenol

5 g of compound obtained in the preceding step are heated at 170° C. for 6 h, in the presence of 50 g of pyridine hydrochloride. Ice is added and the crystals formed are filtered. 4.8 g of desired product are obtained after recrystallizing in an ethanol/water mixture (80:20 v/v).

### G/ 4-{2-[4-(2-Methoxy-4-nitro-phenoxy)-phenyl]-benzofuran-5-yloxy}-1-methyl-piperidine

To a suspension of 1 g of NaH in DMF are added 3.8 g of compound obtained in the preceding step, stirred 1 h at AT, then 2.1 g of 2-chloro-5-nitroanisole are added and stirring continued for 40 h at 60° C., then concentrated in vacuo. The residue is redissolved in water, extracted with DCM, the organic layer is dried over MgSO₄, filtered and evaporated. 3.1 g of desired product are obtained after crystallization in TBME.

### H/ 3-Methoxy-4-{4-[5-(1-methyl-piperidin-4-yloxy)-benzofuran-2-yl]-phenoxy}-phenylamine

2.8 g of compound of the preceding step, in solution in 200 ml of ethyl acetate, are treated with hydrogen under AP and at AT, in the presence of 1 g of 5% sulfided platinum on charcoal. The catalyst is filtered and the filtrate evaporated in vacuo. 2.4 g of desired product are obtained.

### I/ 1-(1-Ethyl-propyl)-3-(3-methoxy-4-{4-[5-(1-methyl-piperidin-4-yloxy)-benzofuran-2-yl]-phenoxy}-phenyl)-urea

The compound of the preceding step is treated following General Procedure H. The desired product is isolated in hydrochloride form after purification by semi-preparative HPLC, following the operating mode described in Example 19.

### Example 251

### 4-(1-Butyl-piperidin-4-yloxy)-N-(4-{2-chloro-4-[3-(1-ethyl-propyl)-ureido]-phenoxy}-2-fluoro-phenyl)-3-methyl-benzamide

4-(1-Butyl-piperidin-4-yloxy)-3-methyl-benzotriazol-1-yl benzoate (2 eq), 360 mg of 1-[4-(4-Amino-3-fluoro-phenoxy)-3-chloro-phenyl]-3-(1-ethyl-propyl)-urea are placed in solution in 8 mL DMF at AT, the solvent is evaporated in vacuo at 60° C. and the mixture held in vacuo at 60° C. for 1 h then at AT for 12 h. 5 mL of DCM/H₂O/TFA mixture (1:1:0.1 v/v/v) is added to the reaction medium and stirred 1 h at AT. After evaporation, the desired product is isolated in hydrochloride form after purification of the reaction medium by semi-preparative HPLC, followed by treatment with a HCl/diethyl ether mixture according to the operating mode described in Example 19.

### Example 252

### 4-(1-Butyl-piperidin-4-yloxy)-N-(4-{4-[3-(1-ethyl-propyl)-ureido]-phenoxy}-phenyl)-N-(2-methoxy-ethyl)-3-methyl-benzamide

4-(1-Butyl-piperidin-4-yloxy)-3-methyl-benzotriazol-1-yl benzoate (1.8 eq), 212 mg of 1-(1-Ethyl-propyl)-3-{4-[4-(2-methoxy-ethylamino)-phenoxy]-phenyl}-urea are placed in solution in 8 mL DMF at AT, the solvent is evaporated in vacuo at 60° C. and the mixture held in vacuo at 60° C. for 1 h and then at AT for 12 h. The reaction medium is redissolved in DCM, washed with a saturated aqueous Na₂CO₃ solution, and the organic layer is dried over MgSO₄, filtered and the filtrate concentrated. The desired product is isolated in hydrochloride form after purifying the reaction medium by semi-preparative HPLC, followed by treatment with a HCl/diethyl ether mixture according to the operating mode described in Example 19.

### Example 253

### 4-(1-Butyl-piperidin-4-yloxy)-N-(4-{4-[3-(1-ethyl-propyl)-ureido]-2-methoxy-phenoxy}-phenyl)-N-(2-methoxy-ethyl)-3-methyl-benzamide

The desired product is obtained by reaction of 4-(1-Butyl-piperidin-4-yloxy)-3-methyl-benzotriazol-1-yl benzoate with 1-(1-Ethyl-propyl)-3-{3-methoxy-4-[4-(2-methoxy-ethylamino)-phenoxy]-phenyl}-urea following the operating mode described in Example 252.

### Example 254

### 4-(1-Butyl-piperidin-4-yloxy)-N-(4-{4-[3-(1-ethyl-propyl)-ureido]-phenoxy}-2-fluoro-phenyl)-3-methoxy-benzamide

116 mg of 1-[4-(4-Amino-3-fluoro-phenoxy)-phenyl]-3-(1-ethyl-propyl)-urea and 4-(1-Butyl-piperidin-4-yloxy)-3-methoxy-benzotriazol-1-yl benzoate obtained such as described in Example 24, are placed in solution in 2 mL DMF at AT. The solvent is evaporated in vacuo at 60° C. and the residue is held in vacuo at 60° C. for 1 h then 6 h at AT. The residue is redissolved in DCM, washed with water, the organic layer is dried over MgSO₄, filtered and the filtrate evaporated. The desired product is isolated in TFA salt form following the operating mode described in Example 1.

### Example 255

### 4-(1-Butyl-piperidin-4-yloxy)-N-(4-{4-[3-(1-ethyl-propyl)-ureido]-3-fluoro-phenoxy}-phenyl)-3-methoxy-benzamide

148 mg of 1-[4-(4-Amino-phenoxy)-2-fluoro-phenyl]-3-(1-ethyl-propyl)-urea and the 4-(1-Butyl-piperidin-4-yloxy)-3-methoxy-benzotriazol-1-yl benzoate are placed in solution in 2 mL DMF at AT, and stirred for 4 days at AT. The residue is redissolved in water, filtered and the precipitate washed with water. The desired product is isolated in TFA salt form after chromatography on silica eluting with a DCM/MeOH/NH₄OH mixture (95:5:0.5 v/v/v), followed by semi-preparative HPLC.

### Example 256

### N-(4-{2-Ethoxyl-4-[3-(1-ethyl-propyl)-ureido]-phenoxy}-phenyl)-3-methyl-4-(1-methyl-piperidin-4-yloxy)-benzamide

### A/ 3-Methyl-4-(1-methyl-piperidin-4-yloxy)-benzotriazol-1-yl benzoate

A mixture of 418 mg of 3-Methyl-4-(1-methyl-piperidin-4-yloxy)-benzoic acid, 350 mg TBTU, 147 mg HOBT and 0.43 mL of DIEA in 10 mL DCM is stirred for 1 h at AT. The desired product is isolated following the operating mode described in Example 1, step A.

### B/ N-(4-{2-Ethoxy-4-[3-(1-ethyl-propyl)-ureido]-phenoxy}-phenyl)-3-methyl-4-(1-methyl-piperidin-4-yloxy)-benzamide

The product obtained in the preceding step and 200 mg of 1-[4-(4-Amino-phenoxy)-3-ethoxy-phenyl]-3-(1-ethyl-propyl)-urea are placed in solution in 8 mL DMF at AT, the solvent is evaporated in vacuo at 60° C. and the mixture held in vacuo at 60° C. for 1 h. The reaction medium is treated and the desired product is isolated in hydrochloride form, following the operating mode described in Example 251.

### Example 257

### N-(4-{4-[3-(1-Ethyl-propyl)-ureido]-3-fluoro-phenoxy}-phenyl)-3-methyl-4-(1-methyl-piperidin-4-yloxy)-benzamide

74 mg of 1-[4-(4-Amino-phenoxy)-2-fluoro-phenyl]-3-(1-ethyl-propyl)-urea and 3-Methyl-4-(1-methyl-piperidin-4-yloxy)-benzotriazol-1-yl benzoate (1.2 eq) are placed in solution in 5 mL DMF at AT, the solvent is evaporated in vacuo at 60° C. and the mixture held in vacuo at 60° C. for 1 h. The desired product is isolated in hydrochloride form following the operating mode described in Example 19.

Following the same operating mode as described in Example 257, the following compounds are obtained:

### N-(4-{4-[3-(1-Ethyl-propyl)-ureido]-phenoxy}-2-fluoro-phenyl)-3-methyl (1-methyl-piperidin-4-yloxy)-benzamide

### Example 258

The desired product is obtained from 3-Methyl-4-(1-methyl-piperidin-4-yloxy)-benzotriazol-1-yl benzoate and 1-[4-(4-Amino-3-fluoro-phenoxy-phenyl]-3-(1-ethyl-propyl)-urea.

### N-(4-{2-Ethoxy-4-[3-(1-ethyl-propyl)-ureido]-phenoxy}-2-fluoro-phenyl)-3-methyl-4-(1-methyl-piperidin-4-yloxy)-benzamide

### Example 259

The desired product is obtained from 3-Methyl(1-methyl-piperidin-4-yloxy)-benzotriazol-1-yl benzoate and 1-[4-(4-Amino-3-fluoro-phenoxy)-3-ethoxy-phenyl]-3-ethyl-propyl)-urea.

### Example 260

### 4-(1-Butyl-piperidin-4-yloxy)-N-(4-{2-ethoxy-4-[3-(1-ethyl-propyl)-ureido]-phenoxy}-2-fluoro-phenyl)-benzamide

73 mg of 1-[4-(4-Amino-3-fluoro-phenoxy)-3-ethoxy-phenyl]-3-(1-ethyl-propyl)-urea and 4-(1-Butyl-piperidin-4-yloxy)-benzotriazol-1-yl benzoate (2.3 eq) are placed in solution in 2 mL DMF at AT, the solvent is evaporated in vacuo at 60° C., the mixture held in vacuo at 60° C. for 1 h then at AT for 48 h. The desired product is isolated in free base form after chromatography on silica eluting with a DCM/MeOH/NH₄OH mixture (95:5:0.5 v/v/v), followed by precipitation with isopropyl ether and washing with pentane.

### Example 261

### 4-(1-Butyl-piperidin-4-yloxy)-N-(4-{2-ethoxy-4-[3-(1-ethyl-propyl)-ureido]-phenoxy}-phenyl)-N-(2-methoxy-ethyl)-benzamide

208 mg of 1-{3-Ethoxy-4-[4-(2-methoxy-ethylamino)-phenoxy]-phenyl}-3-(1-ethyl-propyl)-urea and 4-(1-Butyl-piperidin-4-yloxy)-benzotriazol-1-yl benzoate (1.2 eq) are placed in solution in 2 mL DMF at AT. The solvent is evaporated in vacuo at 60° C. and the residue held in vacuo at 60° C. for 4 h, then 12 h at AT. The residue is redissolved in DCM, washed with water, with a saturated aqueous Na₂CO₃ solution, then with water, the organic layer is dried over MgSO₄, filtered and the filtrate evaporated. The desired product is isolated in hydrochloride form after chromatography on silica eluting with a DCM/MeOH/NH₄OH mixture (95:5:0.5 v/v/v), followed by treatment with a HCl/diethyl ether mixture and washing of the precipitate obtained in ethyl ether.

### Example 262

### 4-(1-Butyl-piperidin-4-yloxy)-N-(2-ethoxy-ethyl)-N-(4-{4-[3-(1-ethyl-propyl)-ureido]-2-methoxy-phenoxy}-phenyl)-benzamide

4-(1-Butyl-piperidin-4-yloxy)-3-methyl-benzotriazol-1-yl benzoate (1.5 eq), 275 mg of 1-{4-[4-(2-Ethoxy-ethylamino)-phenoxy]-3-methoxy-phenyl}-3-(1-ethyl-propyl)-urea are placed in solution in 8 mL DMF at AT, the solvent is evaporated in vacuo at 60° C. and the mixture held in vacuo at 60° C. for 1 h, then at AT for 12 h. To the reaction medium is added 5 mL of DCM/H₂O/TFA mixture (1:1:0.1 v/v/v) followed by stirring for 1 h at AT. The reaction medium is concentrated to dryness, the residue redissolved in DCM, washed with a saturated aqueous Na₂CO₃ solution, and the organic layer is dried over MgSO₄, filtered and the filtrate concentrated. The desired product is isolated in hydrochloride form, following the operating mode described in Example 261.

Following the same operating mode as described in Example 262, the following compounds are obtained:

### 4-(1-Butyl-piperidin-4-yloxy)-N-(4-{4-[3-(1-ethyl-propyl)-ureido]-5-fluoro-2-methoxy-phenoxy}-phenyl)-benzamide

### Example 263

The desired product is obtained from 4-(1-Butyl-piperidin-4-yloxy)-3-methyl-benzotriazol-1-yl benzoate and 1-[4-(4-Amino-phenoxy)-2-fluoro-5-methoxy-phenyl]-3-(1-ethyl-propyl)-urea.

### 4-(1-Butyl-piperidin-4-yloxy)-N-(4-{4-[3-(1-ethyl-propyl)-ureido]-2,5-difluoro-phenoxy}-phenyl)-benzamide

### Example 264

The desired product is obtained from 4-(1-Butyl-piperidin-4-yloxy)-3-methyl-benzotriazol-1-yl benzoate and 1-[4-(4-Amino-phenoxy)-2,5-difluoro-phenyl]-3-(1-ethyl-propyl)-urea.

### Example 265

### 1-(1-Methyl-piperidin-4-yl)-1H-indole-5-carboxylic acid (4-{4-[3-(1-ethyl-propyl)-ureido]-2-methoxy-phenoxy}-phenyl)-amide

### A/ 1-(1-Methyl-piperidin-4-yl)-1H-indole-5-benzotriazol-1-yl carboxylate

A mixture of 1.55 g of 11-Methyl-piperidin-4-yl)-1H-indole-5-carboxylic acid, 1.25 g TBTU, 527 mg HOBT and 1.56 mL of DIEA in 20 mL DCM is stirred for 1 h at AT. The desired product is isolated following the operating mode described in Example 1, step A.

### B/ 1-(1-Methyl-piperidin-4-yl)-1H-indole-5-carboxylic acid (4-{4-[3-(1-ethyl-propyl)-ureido]-2-methoxy-phenoxy}-phenyl)-amide

344 mg of 1-[4-(4-Amino-phenoxy)-3-methoxy-phenyl]-3-(1-ethyl-propyl)-urea, 1.5 eq of product obtained in the preceding step are placed in solution in 8 mL DMF at AT, the solvent is evaporated in vacuo at 60° C. and the mixture held in vacuo at 60° C. for 1 h. After evaporation, the desired product is isolated in hydrochloride form after purifying the reaction medium by semi-preparative HPLC, followed by treatment with a HCl/diethyl ether mixture following the operating mode described in Example 19.

### Example 266

### 1-(1-Methyl-piperidin-4-yl)-1H-indole-5-carboxylic acid (4-{4-[3-(1-ethyl-propyl)-ureido]-phenoxy}-3-methoxymethyl-phenyl)-amide

The desired product is obtained from 1-(1-Methyl-piperidin-4-yl)-1H-indole-5-benzotriazol-1-yl carboxylate and 1-[4-(4-Amino-2-methoxymethyl-phenoxy)-phenyl]-3-(1-ethyl-propyl)-urea following the operating mode described in Example 265.

### Example 267

### 1-(1-Butyl-piperidin-4-yl)-1H-indole-5-carboxylic acid (4-{4-[3-(1-ethyl-propyl)-ureido]-5-fluoro-2-methoxy-phenoxy}-phenyl)-amide

### A/ 1-(1-Butyl-piperidin-4-yl)-1H-indole-5-benzotriazol-1-yl carboxylate

A mixture of 249 mg of 1-(1-Butyl-piperidin-4-yl)-1H-indole-5-carboxylic acid, 346 mg TBTU, 146 mg HOBT and 0.43 mL of DIEA 5 mL DCM is stirred for 1 h at AT. The desired product is isolated by following the operating mode described in Example 1, step A.

### B/ 1-(1-Butyl-piperidin-4-yl)-1H-indole-5-carboxylic acid (4-{4-[3-(1-ethyl-propyl)-ureido]-5-fluoro-2-methoxy-phenoxy}-phenyl)-amide

The desired product is obtained from 200 mg of 1-[4-(4-Amino-phenoxy)-2-fluoro-5-methoxy-phenyl]-3-(1-ethyl-propyl)-urea and the compound obtained in the preceding step (1.5 eq), following the operating mode described in Example 265, step B.

¹H NMR: 10.5 (s, 1H); 10.10 (s, 1H); 8.28 (m, 2H); 8.20 (d, 2H); 7.8 (d, 2H); 7.75-7.70 (m; 3H); 7.5 (d, 1H); 7.00 (d, 1H); 6.83 (d, 2H); 6.67 (d, 1H); 6.5 (d, 1H); 4.85-4.75 (m, 1H); 3.68 (s; 3H); 3.67-3.62 (m, 2H); 3.55-3.40 (m, 1H); 3.22-3.12 (m, 1H); 3.11-3.01 (m, 1H); 2.49 (m, 2H); 2.22-2.15 (m, 2H); 1.79-1.65 (m, 2H); 1.55-1.43 (m, 2H); 1.43-1.40 (m, 4H); 0.94 (t, 3H); 0.87 (t, 6H).

MS (APCI⁺): 644 (M+H)⁺

Elemental analysis: found C, 63.40. H, 7.06. N, 9.92. calculated for C₃₇H₄₆FN₅O₄.1HCl.1H₂O, C, 63.64. H, 7.07. N, 10.03

### Example 268

### 1-(1-Butyl-piperidin-4-yl)-1H-indole-5-carboxylic acid (4-{2-ethoxy-4-[3-(1-ethyl-propyl)-ureido]-phenoxy}-2-fluoro-phenyl)-amide

250 mg of 1-[4-(4-Amino-3-fluoro-phenoxy)-3-ethoxy-phenyl]-3-(1-ethyl-propyl)-urea and 1-(1-Butyl-piperidin-4-yl)-1H-indole-5-benzotriazol-1-yl carboxylate (1 eq) are placed in solution in 15 mL DMF at AT. The solvent is evaporated in vacuo at 60° C. and the residue is held in vacuo at 60° C. for 4 h, then 12 h at AT. The residue is redissolved in ethyl acetate, washed with water, the organic layer is dried over MgSO₄, filtered and the filtrate evaporated. The desired product is isolated in hydrochloride form after chromatography on silica eluting with a DCM/MeOH/NH₄OH mixture (95:5:0.5 v/v/v), followed by treatment with a HCl/diethyl ether mixture and washing of the precipitate obtained in ethyl acetate.

### Example 269

### 1-(1-Butyl-piperidin-4-yl)-1H-indole-5-carboxylic acid (4-{2-ethoxy-4-[3-(1-ethyl-propyl)-ureido]-phenoxy}-phenyl)-amide

250 mg of 1-[4-(4-Amino-phenoxy)-3-ethoxy-phenyl]-3-(1-ethyl-propyl)-urea, 1-(1-Butyl-piperidin-4-yl)-1H-indole-5-benzotriazol-1-yle_carboxylate (1.1 eq) are placed in solution in 2 mL DMF at AT, the solvent is evaporated in vacuo at 60° C., the mixture held in vacuo at 60° C. for 1 h and 48 h at AT. The desired product is isolated in free base form after chromatography on silica eluting with a DCM/MeOH/NH₄OH mixture (95:5:0.5 v/v/v), followed by precipitation with acetone and washing of the precipitate thus obtained with pentane.

### Example 270

### 4-(1-Butyl-piperidin-4-yloxy)-N-(4-{4-[3-(1-ethyl-propyl)-ureido]-5-fluoro-2-methoxy-phenoxy}-phenyl)-2,5-difluoro-benzamide

### A/ 4-(1-Butyl-piperidin-4-yloxy)-2,5-difluoro-benzotriazol-1-yl benzoate

A mixture of 420 mg of 4-(1-Butyl-piperidin-4-yloxy)-2,5-difluoro-benzoic acid, 502 mg TBTU, 211 mg HOBT and 0.79 mL of DIEA in 40 mL DCM is stirred 1 h at AT. The desired product is isolated following the operating mode described in Example 1, step A.

### B/ 4-(1-Butyl-piperidin-4-yloxy)-N-(4-{4-[3-(1-ethyl-propyl)-ureido]-5-fluoro-2-methoxy-phenoxy}-phenyl)-2,5-difluoro-benzamide

A mixture of 2 mL of DMF, 157 mg of 1-[4-(4-Amino-phenoxy)-2-fluoro-5-methoxy-phenyl]-3-(1-ethyl-propyl)-urea and 4-(1-Butyl-piperidin-4-yloxy)-2,5-difluoro-benzotriazol-1-yl benzoate (1.4 eq) is stirred 12 h at AT. After concentrating to dryness, the residue is redissolved in ethyl acetate, washed with water, with a saturated aqueous Na₂CO₃ solution, then with water, and the organic layer is dried over MgSO₄, filtered and the filtrate evaporated. The desired product is isolated in hydrochloride form after chromatography on silica eluting with a DCM/MeOH/NH₄OH mixture (94:6:0.6 v/v/v), followed by treatment with a HCl/diethyl ether mixture and washing of the precipitate thus obtained with pentane.

### Example 271

### 4-(1-Butyl-piperidin-4-yloxy)-N-(4-{4-[3-(1-ethyl-propyl)-ureido]-2,5-difluoro-phenoxy}-phenyl)-2,5-difluoro-benzamide

The desired product is obtained from 4-(1-Butyl-piperidin-4-yloxy)-2,5-difluoro-benzotriazol-1-yl benzoate and 1-[4-(4-Amino-phenoxy)-2,5-difluoro-phenyl]-3-(1-ethyl-propyl)-urea following the operating mode described in Example 270.

### Example 272

### 4-(1-Butyl-piperidin-4-yloxy)-N-(4-{2-ethoxy-4-[3-(1-ethyl-propyl)-ureido]-phenoxy}-phenyl)-2,5-difluoro-benzamide

Method 1: The desired product is obtained from 4-(1-Butyl-piperidin-4-yloxy)-2,5-difluoro-benzotriazol-1-yl benzoate and 1-[4-(4-Amino-phenoxy)-3-ethoxy-phenyl]-3-(1-ethyl-propyl)-urea following the operating mode described in Example 270, with direct chromatography of the reaction medium.
Method 2: 500 mg of 4-(1-Butyl-piperidin-4-yloxy)-2,5-difluoro-benzoic acid are heated 1 h at 58° C. with 6 mL of oxalyl chloride. The reaction medium is evaporated in vacuo, to the formed acid chloride is added 10 mL of DMF, DIEA (920 μL) and 1-[4-(4-Amino-phenoxy)-3-ethoxy-phenyl]-3-(1-ethyl-propyl)-urea (523 mg) and stirred 30 min at AT. The desired product is isolated in free base form after chromatography on silica eluting with a DCM/MeOH/NH₄OH mixture (95:5:0.5 v/v/v). The hydrochloride is obtained by treating with a HCl/diethyl ether mixture.

¹H NMR: 10.28 (s, 1H); 10.19 (s, 1H); 8.50 (s, 1H); 7.62-7.57 (m, 3H); 7.48-7.42 (m, 1H); 7.39 (d; 1H); 6.89 (d, 1H); 6.90-6.78 (m, 3H); 6.04 (d, 2H); 4.90 (m, 0.6H); 4.70 (m, 0.4H); 3.95 (q, 2H); 3.56 (m; 1H); 3.48-3.37 (m, 1H); 3.12-2.97 (m, 4H); 2.28 (m, 1H); 2.17 (m, 1H); 2.09 (m, 1H); 1.95 (m, 1H); 1.71-1.64 (m, 2H); 1.50-1.40 (m, 2H); 1.45-1.34 (m, 4H); 1.18,(t, 3H); 0.92 (t 3H); 0.88 (t, 6H).

MS (APCI⁺): 653 (M+H)⁺

Elemental analysis: found C, 62.02. H, 6.93. N, 7.96. calculated for C₃₆H₄₆F₂N₄O₅.1HCl.0,5H₂O, C, 61.93. H, 6.93. N, 8.02

### Example 273

### 4-(1-Butyl-3-fluoro-piperidin-4-yloxy)-N-(4-{2-ethoxy-4-[3-(1-ethyl-propyl)-ureido]-phenoxy}-phenyl)-benzamide

### A/ 4-(1-Butyl-3-fluoro-piperidin-4-yloxy)-benzotriazol-1-yl benzoate

A mixture of 185 mg of 4-(1-Butyl-3-fluoro-piperidin-4-yloxy)-benzoic acid, 261 mg TBTU, 109 mg HOBT and 0.41 mL of DIEA in 10 mL DCM is stirred 1 h at AT. The desired product is isolated following the operating mode described in Example 1, step A.

### B/ 4-(1-Butyl-3-fluoro-piperidin-4-yloxy)-N-(4-{2-ethoxy-4-[3-(1-ethyl-propyl)-ureido]-phenoxy}-phenyl)-benzamide

### 143 mg of 1-[4-(4-Amino-phenoxy)-3-ethoxy-phenyl]-3-(1-ethyl-propyl)-urea are placed in solution in 3 mL of DMF at AT with the compound prepared in the preceding step. The solvent is evaporated in vacuo at 60° C. and the residue held in vacuo at 60° C. for 12 h. The desired product is isolated in hydrochloride form after chromatography on silica eluting with a DCM/MeOH/NH₄OH mixture (95:5:0.5 v/v/v), followed by treatment with a HCl/diethyl ether mixture and washing of the precipitate thus formed with ethyl ether.

### Example 274

### 4-(1-Dimethylamino-piperidin-4-yloxy)-N-(4-{2-ethoxy-4-[3-(1-ethyl-propyl)-ureido]-phenoxy}-phenyl)-benzamide

### A/ 1-Dimethylamino-piperidin-4-yloxy)-benzotriazol-1-yl benzoate

A mixture of 400 mg of 4-(1-Dimethylamino-piperidin-4-yloxy)-benzoic acid, 470 mg TBTU, 202 mg HOBT and 0.57 mL DIEA in 5 mL DCM is stirred for 1 h at AT. The desired product is isolated following the operating mode described in Example 1, step A.

### B/ 4-(1-Dimethylamino-piperidin-4-yloxy)-N-(4-{2-ethoxy-4-[3-(1-ethyl-propyl)-ureido]-phenoxy}-phenyl)-benzamide

250 mg of 1-[4-(4-Amino-phenoxy)-3-ethoxy-phenyl]-3-(1-ethyl-propyl)-urea and 500 mg of compound obtained in the preceding step are placed in solution in 2 mL DMF at AT, the solvent is evaporated in vacuo at 60° C., the mixture held in vacuo at 60° C. for 1 h and 48 h at AT. The reaction medium is redissolved in DCM, washed with water, and the organic layer is dried over MgSO₄, filtered and the filtrate concentrated. The desired product is isolated in free base form, after chromatography on silica eluting with a DCM/MeOH/NH₄OH mixture (95:5:0.5 v/v/v), followed by precipitation with isopropyl ether, washing with acetone and with pentane.

### Example 275

### 4-(1-Butyl-pyrrolidin-3-yloxy)-N-(4-{2-ethoxy-4-[3-(1-ethyl-propyl)-ureido]-phenoxy}-phenyl)-benzamide

### A/ 4-(1-Butyl-pyrrolidin-3-yloxy)-benzotriazol-1-yl benzoate

A mixture of 200 mg of 4-(1-Butyl-pyrrolidin-3-yloxy)-benzoic acid, 278 mg of TBTU, 116 mg HOBT and 0.35 mL of DIEA in 20 mL DCM is stirred 1 h at AT. The desired product is isolated following the operating mode described in Example 1, step A.

### B/ 4-(1-Butyl-pyrrolidin-3-yloxy)-N-(4-{2-ethoxy-4-[3-(1-ethyl-propyl)-ureido]-phenoxy}-phenyl)-benzamide

230 mg of 1-[4-(4-Amino-phenoxy)-3-ethoxy-phenyl]-3-(1-ethyl-propyl)-urea and the compound obtained in the preceding step are placed in solution in 2 mL DMF at AT, the solvent is evaporated in vacuo at 60° C., the mixture held in vacuo at 60° C. for 1 h and 12 h at AT. The desired product is isolated in hydrochloride form after chromatography on silica eluting with a DCM/MeOH/NH₄OH mixture (95:5:0.5 v/v/v), followed by treatment with a HCl/diethyl ether mixture and washing of the precipitate thus obtained with water and with pentane.

### Example 276

### 4-[(1-Butyl-piperidin-4-yl)-methyl-amino]-N-(4-{4-[3-(1-ethyl-propyl)-ureido]-2-methoxy-phenoxy}-phenyl)-benzamide

### A/ 4-[(1-Butyl-piperidin-4-yl)-methyl-amino]-benzotriazol-1-yl benzoate

A mixture of 500 mg of 4-[(1-Butyl-piperidin-4-yl)-methyl-amino]-benzoic acid, 719 mg TBTU, 302 mg HOBT and 1.49 mL of DIEA in 220 mL DCM is stirred 1 h at AT. The reaction medium is washed with a 1 N solution of KOH, with water, and the organic layer is dried over MgSO₄, filtered and the filtrate concentrated.

### B/ 4-[(1-Butyl-piperidin-4-yl)-methyl-amino]-N-(4-{4-[3-(1-ethyl-propyl)-ureido]-2-methoxy-phenoxy}-phenyl)-benzamide

The compound obtained in the preceding step is diluted in 9 mL DMF, 6 mL of this solution are added to 263 mg of 1-[4-(4-Amino-phenoxy)-3-methoxy-phenyl]-3-(1-ethyl-propyl)-urea, the solvent is evaporated in vacuo at 60° C., the mixture held in vacuo at 60° C. for 1 h and at AT for 12 h. The reaction medium is redissolved in water and ethyl acetate, filtered and the precipitate washed with a dilute aqueous solution of NaHCO₃. The organic layer is washed with a dilute NaHCO₃ solution, with water, and the organic layer dried over MgSO₄, filtered and concentrated to dryness. The residue obtained is added to the precipitate and the mixture purified by chromatography on silica eluting with a DCM/MeOH/NH₄OH mixture (95:5:0.5 v/v/v), followed by treatment with a HCl/diethyl ether mixture and washing of the precipitate thus obtained with ethyl ether.

### Example 277

### 4-[(1-Butyl-piperidin-4-yl)-methyl-amino]-N-(4-{4-[3-(1-ethyl-propyl)-ureido]-5-fluoro-2-methoxy-phenoxy}-phenyl)-benzamide

4-[(1-Butyl-piperidin-4-yl)-methyl-amino]-benzotriazol-1-yl benzoate (2 eq) and the 1-[4-(4-Amino-phenoxy)-2-fluoro-5-methoxy-phenyl]-3-(1-ethyl-propyl)-urea are placed in solution in 8 mL DMF at AT, the solvent is evaporated in vacuo at 60° C. and the mixture held in vacuo at 60° C. for 1 h then at AT for 12 h. To the reaction medium is added 10 mL of a DCM/H₂O/TFA mixture (1:1:0.1) and stirred 1 h at AT. The reaction medium is concentrated to dryness, the residue is redissolved in DCM, washed with a dilute aqueous K₂CO₃ solution, and the organic layer is dried over Na₂SO₄, filtered and the filtrate concentrated. The desired product is isolated in hydrochloride form after chromatography on silica eluting with an ethyl acetate/MeOH/NH₄OH mixture (95:5:0.5 v/v/v), followed by treatment with a HCl/diethyl ether mixture and washing of the precipitate thus obtained with ethyl ether.

### Example 278

### 4-[(1-Butyl-piperidin-4-yl)-methyl-amino]-N-(4-{4-[3-(1-ethyl-propyl)-ureido]-3-fluoro-phenoxy}-phenyl)-benzamide

The desired product is obtained from 4-[(1-Butyl-piperidin-4-yl)-methyl-amino]-benzotriazol-1-yl benzoate (1.5 eq) and 1-[4-(4-Amino-phenoxy)-2-fluoro-phenyl]-3-(1-ethyl-propyl)-urea following the operating mode described in Example 275, step B.

### Example 279

### 4-(1-Butyl-piperidin-4-yloxy)-2-chloro-N-(4-{4-[3-(1-ethyl-propyl)-ureido]-2-methoxy-phenoxy}-phenyl)-benzamide

Following General Procedure 1,327 mg of 4-(1-Butyl-piperidin-4-yloxy)-2-chloro-benzoic acid are activated in the presence of a TBTU/HOBT mixture for 30 min at AT, 240 mg of 1-[4-(4-Amino-phenoxy)-3-methoxy-phenyl]-3-(1-ethyl-propyl)-urea are added and stirred 48 h at AT. After evaporation in vacuo the desired product is isolated in TFA salt form after purification by semi-preparative HPLC.

### Example 280

### 1-(1-Butyl-piperidin-4-yl)-2,3-dihydro-1H-indole-5-carboxylic acid (4-{2-ethoxyl-4-[3-(1-ethyl-propyl)-ureido]-phenoxy}-phenyl)-amide

Following General Procedure I, 40 mg of 1-(1-Butyl-piperidin-4-yl)-2,3-dihydro-1H-indole-5-carboxylic acid are activated in the presence of a TBTU/HOBT mixture for 30 min at AT, 106 mg of 1-[4-(4-Amino-phenoxy)-3-ethoxy-phenyl]-3-(1-ethyl-propyl)-urea are added, the solvent is evaporated in vacuo at 60° C. and the mixture held in vacuo at 60° C. for 1 h and at AT for 12 h. The desired product is isolated in TFA salt form, after purification by semi-preparative HPLC.

### Example 281

### 4 (1-Butyl-piperidin-4-yloxy)-N-(4-{4-[3-(1-ethyl-propyl)-ureido]-2-methoxy-phenoxy}-phenyl)-2-methyl-benzamide

Following General Procedure L1, 300 mg of 4-(1-Butyl-piperidin-4-yloxy)-2-methyl-benzoic acid are activated in the presence of a EDCI/HOBT mixture, 378 mg of 1-[4-(4-Amino-phenoxy)-3-methoxy-phenyl]-3-(1-ethyl-propyl)-urea are added and stirred 48 h at AT. After evaporating the solvent in vacuo, the desired product is isolated in hydrochloride form after chromatography on silica eluting with a DCM/MeOH/NH₄OH mixture (95:5:0.5 v/v/v), followed by treatment with a HCl/diethyl ether mixture and washing of the precipitate formed with diisopropyl ether.

### Example 283

### N-(4-{2-Ethoxy-4-[3-(1-ethyl-propyl)-ureido]-phenoxy}-phenyl)-4-(4-ethyl-piperazin-1-yl)-benzamide

Following General Procedure L1, the desired product is obtained from 4-(4-ethyl-piperazin-1-yl)-benzoic acid and 1-[4-(4-Amino-phenoxy)-3-ethoxy-phenyl]-3-ethyl-propyl)-urea. The desired product is isolated in hydrochloride form after chromatography on silica eluting with a DCM/MeOH/NH₄OH mixture (95:5:0.5 v/v/v), followed by treatment with a HCl/diethyl ether mixture and washing of the precipitate thus obtained with ethyl ether.

Following the same operating mode as described in Example 283, the following compounds are obtained:

### N-(4-{2-Ethoxy-4-[3-(1-ethyl-propyl)-ureido]-phenoxy}-phenyl)-4-(4-methyl-[1,4]diazepan-1-yl)-benzamide

### Example 282

The desired product is obtained from 4-(4-Methyl-[1,4]diazepan-1-yl)-benzoic acid and 1-[4-(4-Amino-phenoxy)-3-ethoxy-phenyl]-3-(1-ethyl-propyl)-urea. The desired product is isolated in hydrochloride form after chromatography on silica eluting with a DCM/MeOH/NH₄OH mixture (95:5:0.5 v/v/v), redissolving in MeOH and precipitation with 5 N HCl in isopropanol.

### 1-(1-Butyl-piperidin-4-yl)-1H-indole-5-carboxylic acid (4-{4-[3-(1-ethyl-propyl)-ureido]-phenoxy}-phenyl)-amide

### Example 284

The desired product is obtained from 1-(1-Butyl-piperidin-4-yl)-1H-indole-5-carboxylic acid and 1-[4-(4-Amino-phenoxy)-phenyl]-3-(1-ethyl-propyl)-urea. The desired product is isolated in free base form after chromatography on silica eluting with a DCM/MeOH/NH₄OH mixture (95:5:0.5 v/v/v).

### 4-(4-Butyl-piperazin-1-yl)-N-(4-{2-ethoxy-4-[3-(1-ethyl-propyl)-ureido]-phenoxy}-phenyl)-benzamide

### Example 285

The desired product is obtained from 4-(4-Butyl-piperazin-1-yl)-benzoic acid and 1-[4-(4-Amino-phenoxy)-3-ethoxy-phenyl]-3-(1-ethyl-propyl)-urea.

### 4-(4-Butyl-[1,4]diazepan-1-yl)-N-(4-{4-[3-(1-ethyl-propyl)-ureido]-5-fluoro-2-methoxy-phenoxy}-phenyl)-benzamide

### Example 286

The desired product is obtained from 4-(4-Butyl-[1,4]diazepan-1-yl)-benzoic acid and 1-[4-(4-Amino-phenoxy)-2-fluoro-5-methoxy-phenyl]-3-(1-ethyl-propyl)-urea.

### 4-(4-Butyl-[1,4]diazepan-1-yl)-N-(4-{4-[3-(1-ethyl-propyl)-ureido]-2-methoxy-phenoxy}-phenyl)-benzamide

### Example 287

The desired product is obtained from 4-(4-Butyl-[1,4]diazepan-1-yl)-benzoic acid and 1-[4-(4-Amino-phenoxy)-3-methoxy-phenyl]-3-(1-ethyl-propyl)-urea.

### 4-(1-Butyl-piperidin-4-yloxy)-N-(4-{4-[3-(1-ethyl-propyl)-ureido]-3-fluoro-phenoxy}-phenyl)-3-methyl-benzamide

### Example 288

The desired product is obtained from 4-(1-Butyl-piperidin-4-yloxy)-3-methyl-benzoic acid and 1-[4-(4-Amino-phenoxy)-2-fluoro-phenyl]-3-(1-ethyl-propyl)-urea, activation of the acid and coupling with the amine being conducted in DCM as solvent instead of DMF. The reaction medium is washed with a saturated aqueous NaCl solution, the organic layer dried over MgSO₄, filtered and concentrated. The desired product is isolated in hydrochloride form after chromatography on silica eluting with a DCM/MeOH/NH₄OH mixture (95:5:0.5 v/v/v), followed by treatment with a HCl/diethyl ether mixture and washing of the precipitate thus obtained with ethyl acetate.

### 4-(1-Butyl-piperidin-4-yloxy)-N-(4-{2-(2-dimethylamino-ethoxy)-4-[3-(1-ethyl-propyl)-ureido]-phenoxy}-2-fluoro-phenyl)-benzamide

### Example 289

The desired product is obtained from 4-(1-Butyl-piperidin-4-yloxy)-benzoic acid and 1-[4-(4-Amino-3-fluoro-phenoxy)-3-(2-dimethylamino-ethoxy phenyl]-3-(1-ethyl-propyl)-urea. The reaction medium is washed with a saturated aqueous NaCl solution; the organic layer is dried over MgSO₄, filtered and concentrated. The desired product is isolated in TFA salt form after chromatography on silica eluting with a DCM/MeOH/NH₄OH mixture (95:5:0.5 v/v/v), followed by semi-preparative HPLC.

### Example 290

### 1-(1-Butyl-piperidin-4-yl)-1H-indole-5-carboxylic acid-(4-{4-[3-(1-ethyl-propyl)-ureido]-phenoxyl}-3-methyl-phenyl)-amide

Following General Procedure L3, 200 mg of 1-(1-Butyl-piperidin-4-yl)-1H-indole-5-carboxylic acid are reacted with 230 mg of 1-[4-(4-Amino-2-methyl-phenoxy)-phenyl]-3-(1-ethyl-propyl)-urea, in the presence of a EDCI/HOBT mixture. The desired product is isolated in hydrochloride form after chromatography on silica eluting with a DCM/MeOH/NH₄OH mixture (95:5:0.5 v/v/v), followed by treatment with a HCl/diethyl ether mixture and washing of the precipitate thus obtained with ethyl acetate.

Following the same operating mode as described in Example 290, the following compounds are obtained:

### 4-(4-Butyl-[1,4]diazepan-1-yl)-N-(4-{2-ethoxy-4-[3-(1-ethyl-propyl-ureido]-phenoxy}-phenyl)-benzamide

### Example 291

The desired product is obtained from 4-(4-Butyl-[1,4]diazepan-1-yl)-benzoic acid and 1-[4-(4-Amino-phenoxy)-3-ethoxy-phenyl]-3-(1-ethyl-propyl)-urea.

### 4-(1-Butyl-piperidin-4-yloxy)-N-(4-{4-[3-(1-ethyl-propyl)-ureido]-5-fluoro-2-methoxy-phenoxy}-phenyl)-2-fluoro-5-methyl-benzamide

### Example 292

The desired product is obtained from 4-(1-Butyl-piperidin-4-yloxy)-2-fluoro-5-methyl-benzoic acid and 1-[4-(4-Amino-phenoxy)-2-fluoro-5-methoxy-phenyl]-3-(1-ethyl-propyl)-urea.

### 4-(1-Butyl-piperidin-4-yloxy)-N-(4-{2-ethoxy-[3-(1-ethyl-propyl)-ureido]-phenoxy}-phenyl)-2-fluoro-5-methyl-benzamide

### Example 293

The desired product is obtained from 4-(1-Butyl-piperidin-4-yloxy)-2-fluoro-5-methyl-benzoic acid and 1-[4-(4-Amino-phenoxy)-3-ethoxy-phenyl]-3-(1-ethyl-propyl)-urea.

### N-(4-{2-Ethoxy-4-[3-(1-ethyl-propyl)-ureido]-phenoxy}-phenyl)-4-(4-ethyl-piperazin-1-ylmethyl)-benzamide

### Example 294

The desired product is obtained from 4-(4-Ethyl-piperazin-1-methyl)-benzoic acid and 1-[4-(4-Amino-phenoxy)-3-ethoxy-phenyl]-3-(1-ethyl-propyl)-urea. The solvent is evaporated in vacuo, the residue redissolved in DCM, washed with an aqueous sodium bicarbonate solution then with water, the organic layer is dried over MgSO₄, filtered and concentrated to dryness. The desired product is isolated in hydrochloride form after chromatography on silica eluting with a DCN/MeOH/NH₄OH mixture (95:5:0.5 v/v/v), followed by treatment with a HCl/diethyl ether mixture and washing of the precipitate thus obtained with diethyl ether.

### 1-(1-Butyl-piperidin-4-yl)-1H-indole-5-carboxylic acid (4-{4-[3-(1-ethyl-propyl)-ureido]-2,5-difluoro-phenoxy}-phenyl)-amide

### Example 295

The desired product is obtained from 1-(1-Butyl-piperidin-4-yl)-1H-indole-5-carboxylic acid and 1-[4-(4-Amino-phenoxy)-2,5-difluoro-phenyl]-3-(1-ethyl-propyl)-urea, conducting the coupling with the amine for 5 days at AT. The desired product is isolated in hydrochloride form after chromatography on silica eluting with a DCM/MeOH/NH₄OH mixture (95:5:0.5 v/v/v), followed by treatment with a HCl/diethyl ether mixture and washing of the precipitate thus obtained with diethyl ether.

### 4-(1-Butyl-piperidin-4-yloxy)-N-(4-{4-[3-(1-ethyl-propyl)-ureido]-2,5-difluoro-phenoxy}-phenyl)-3-methyl-benzamide

### Example 296

The desired product is obtained from 4-(1-Butyl-piperidin-4-yloxy)-3-methyl-benzoic acid and 1-[4-(4-Amino-phenoxy)-2,5-difluoro-phenyl]-3-(1-ethyl-propyl)-urea, conducting coupling with the amine for 4 days at AT. The desired product is isolated in hydrochloride form after chromatography on silica eluting with a DCM/MeOH/NH₄OH mixture (95:5:0.5 v/v/v), followed by treatment with a HCl/diethyl ether mixture and washing of the precipitate thus obtained with diethyl ether.

¹H NMR: 10.5 (s, 1H); 10.07 (s, 1H); 8.46 (s, 1H); 8.20 (m, 1H); 7.83 (m, 2H); 7.22-7.11 (m; 2H); 6.95 (d, 2H); 6.6 (d, 1H); 4.9 (m, 0.6H); 4.68 (m, 0.5H); 3.58-3.39 (m, 2H); 3.09-3.02 (m, 4H); 2.30 (s; 3H); 2.24-2.21 (m, 2H); 2.09 (m, 2H); 2.00-1.9 (m, 1H); 1.69 (m, 2H); 1.55-1.45 (m, 2H); 1.38-1.30 (m, 4H); 0.92 (t, 3H); 0.86 (t, 6H)

MS (APCI⁺): 623 (M+H)⁺

Elemental analysis: found C, 62.07. H, 6.87. N, 8.12. calculated for C₃₅H₄₄F₂N₄O₄.1HCl.1H₂O, C, 62.07. H, 7.00. N, 8.27

### 2-Methyl-1,2,3,4-tetrahydro-benzo[4,5]furo[3,2-c]pyridine-8-carboxylic acid (4-{4-[3-(1-Ethyl-propyl)-ureido]-2-methoxy-phenoxy}-phenyl)-amide

### Example 297

The desired product is obtained from 2-Methyl-1,2,3,4-tetrahydro-benzo[4,5]furo[3,2-c]pyridine-8-carboxylic acid and 1-[4-(4-Amino-phenoxy)-3-methoxy-phenyl]-3-(1-ethyl-propyl)-urea, conducting coupling with the amine for 4 days at AT. The desired product is isolated in hydrochloride form after chromatography on silica eluting with a DCM/MeOH/NH₄OH mixture (95:5:0.5 v/v/v), followed by treatment with a HCl/diethyl ether mixture and washing of the precipitate thus obtained with diethyl ether.

### 4-(1-Butyl-piperidin-4-yloxy)-N-(4-{4-[3-(1-ethyl-propyl)-ureido]-3-fluoro-phenoxy}-3-methyl-phenyl)-benzamide

### Example 298

The desired product is obtained from 4-(1-Butyl-piperidin-4-yloxy)-benzoic acid and 1-[4-(4-Amino-2-methyl-phenoxy)-2-fluoro-phenyl]-3-(1-ethyl-propyl)-urea. The solvent is evaporated in vacuo, the residue redissolved in DCM, washed with an aqueous sodium bicarbonate solution then with water, the organic layer is dried over MgSO₄, filtered and concentrated to dryness. The desired product is isolated in hydrochloride form after chromatography on silica eluting with a DCM/MeOH/NH₄OH mixture (95:5:0.5 v/v/v), followed by treatment with a HCl/diethyl ether mixture and washing of the precipitate thus obtained with diethyl ether.

### 4-(4-Butyl-piperazin-1-yl)-N-(4-{2-ethoxy-4-[3-(1-ethyl-propyl)-ureido]-phenoxy}-phenyl)-2-fluoro-5-methyl-benzamide

### Example 299

The desired product is obtained from 4-(4-Butyl-piperazin-1-yl)-2-fluoro-5-methyl-benzoic acid and 1-[4-(4-Amino-phenoxy)-3-ethoxy-phenyl]-3-ethyl-propyl)-urea, heating the reaction medium 3 h under reflux. The reaction medium is washed with water and a saturated aqueous NaHCO₃ solution, the organic layer is dried over MgSO₄, filtered and the filtrate concentrated. The product is isolated in base form by precipitation in an ethyl acetate/diethyl ether mixture.

### Example 300

### 4-(1-Butyl-piperidin-4-yloxy)-N-(4-{4-[3-(1-ethyl-propyl)-ureido]-2-meth phenoxy}-phenyl)-N-tetrahydro-pyranyl)-benzamide

1 g of 4-(1-Butyl-piperidin-4-yloxy)-benzoic acid is heated 22 h under reflux with 2 mL of thionyl chloride in 20 mL DCE. The reaction medium is evaporated in vacuo. 166 mg of the acid chloride thus formed and TEA (1 eq) in solution in 2 mL DCE are added to a solution of 1-(1-Ethyl-propyl)-3-{3-methoxy-4-[4-tetrahydro-pyran-4-ylamino)-phenoxy]-phenyl}-urea (1 eq) and TEA (2 eq) in 5 mL DCE and heated 3 h at 60° C. After return to AT, the reaction medium is washed with water, with a saturated aqueous NaHCO₃ solution, with water, and the organic layer is dried over MgSO₄, filtered and the filtrate concentrated. The desired product is isolated in free base form after chromatography on silica eluting with a DCM/MeOH/NH₄OH mixture (95:5:0.5 v/v/v). The hydrochloride is obtained by treating with a HCl/diethyl ether mixture.

Following the same operating mode as described in Example 300, the following compounds are obtained:

### 4-(1-Butyl-piperidin-4-yloxy)-N-(4-{4-[3-(1-ethyl-propyl)-ureido]-2-methoxy-phenoxy}-phenyl)-N-(2-methoxy-1-methyl-ethyl)-benzamide

### Example 301

The desired product is obtained from 4-(1-Butyl-piperidin-4-yloxy)-benzoic acid and 1-(1-Ethyl-propyl)-3-{3-methoxy-4-[4-(2-methoxy-1-methyl-ethylamino)-phenoxy]-phenyl}-urea.

### 4-(1-Butyl-piperidin-4-yloxy)-N-(4-{4-[3-(1-ethyl-propyl)-ureido]-2-methoxy-phenoxy}-phenyl)-N-(2-methoxy-propyl)-benzamide

### Example 302

The desired product is obtained from 4-(1-Butyl-piperidin-4-yloxy)-benzoic acid and 1-(1-Ethyl-propyl)-3-{3-methoxy-4-[4-(2-methoxy-propylamino)-phenoxy]-phenyl}-urea, conducting the coupling of the amine in the presence of DIEA (2.2 eq) instead of TEA, in 40 mL DCE for 12 h at AT. The desired product is isolated in hydrochloride form after chromatography on silica eluting with a DCM/MeOH/NH₄OH mixture (96:4:0.4 v/v/v), followed by treatment with a HCl/diethyl ether mixture.

### 4-(1-Butyl-piperidin-4-yloxy)-N-(4-{4-[3-(1-ethyl-propyl)-ureido]-2-methoxy-phenoxy}-phenyl)-N-(tetrahydro-furan-3-yl)-benzamide

### Example 303

The desired product is obtained from 4-(1-Butyl-piperidin-4-yloxy)-benzoic acid and 1-(1-Ethyl-propyl)-3-{3-methoxy-4-[4-(tetrahydro-furan-3-ylamino)-phenoxy]-phenyl}-urea, conducting coupling with the amine in 20 mL DCE for 12 h at AT. The desired product is isolated in hydrochloride form after purification of the reaction medium by semi-preparative HPLC, followed by treatment with a HCL/diethyl ether mixture according to the operating mode described in Example 19.

### 4-(1-Butyl-piperidin-4-yloxy)-N-(4-{4-[3-(1-ethyl-propyl)-ureido]-2-methoxy-phenoxy}-phenyl)-N-tetrahydro-furan-2-ylmethyl)-benzamide

### Example 304

The desired product is obtained from 4-(1-Butyl-piperidin-4-yloxy)-benzoic acid and 1-(1-Ethyl-propyl-3-(3-methoxy-4-{4-[(tetrahydro-furan-2-ylmethyl-amino]-phenoxy}-phenyl)-urea. The desired product is isolated in hydrochloride form after purification of the reaction medium by semi-preparative HPLC, followed by treatment with a HCl/diethyl ether mixture according to the operating mode described in Example 19.

### 4-(1-Butyl-piperidin-4-yloxy)-N-ethyl-N-(4-{4-[3-(1-ethyl-propyl)-ureido]-5-fluoro-2-methoxy-phenoxy}-phenyl)-3-methyl-benzamide

### Example 305

The desired product is obtained from 4-(1-Butyl-piperidin-4-yloxy)-3-methyl-benzoic acid and 1-[4-(4-Ethylamino-phenoxy)-2-fluoro-5-methoxy-phenyl]-3-(1-ethyl-propyl)-urea, using DCM as solvent for formation of the acid chloride and coupling with the amine, and conducting coupling with the amine in the presence of DIEA (1.1 eq) instead of TEA.

### Example 306

### 4-(1-Butyl-piperidin-4-yloxy)-N-(4-{4-[3-(1-ethyl-propyl)-ureido]-5-fluoro-2-methoxy-phenoxy}-phenyl)-N-(2-methoxy-ethyl)-3-methyl-benzamide

234 mg of 4-(1-Butyl-piperidin-4-yloxy)-3-methyl-benzoic acid are heated under reflux for 4 h with 4 mL of thionyl chloride. The reaction medium is evaporated in vacuo. To the acid chloride thus obtained are added 10 mL of DCM, TEA (200 μL) and 1-(1-Ethyl-propyl)-3-{2-fluoro-5-methoxy-4-[4-(2-methoxy-ethylamino)-phenoxy]-phenyl}-urea (1 eq), and stirred 48 h at AT. The reaction medium is washed with water, with a saturated NaHCO₃ solution, with water, and the organic layer is dried over MgSO₄, filtered and the filtrate concentrated. The desired product is isolated in free base form after chromatography on silica eluting with DCM/MeOH/NH₄OH mixture (95:5:0.5 v/v/v). The hydrochloride is obtained by treating with a HCl/diethyl ether mixture.

Following the same operating mode as described in Example 306, the following compounds are obtained:

### 4-(1-Butyl-piperidin-4-yloxy)-N-(4-{2-ethoxy-4-[3-(1-ethyl-propyl)-ureido]-5-fluoro-phenoxy}-phenyl)-3-methyl-benzamide

### Example 307

The desired product is obtained from 4-(1-Butyl-piperidin-4-yloxy)-3-methyl-benzoic acid and 1-[4-(4-Amino-phenoxy)-5-ethoxy-2-fluoro-phenyl]-3-(1-ethyl-propyl)-urea.

### 4-(1-Butyl-piperidin-4-yloxy)-N-{4-[5-fluoro-4-(3-isopropyl-ureido)-2-methoxy-phenoxy]-phenyl}-3-methyl-benzamide

### Example 308

The desired product is obtained in base form from 4-(1-Butyl-piperidin-4-yloxy)-3-methyl-benzoic acid and 1-[4-(4-Amino-phenoxy)-2-fluoro-5-methoxy-phenyl]-3-isopropyl-urea following the operating mode described in Example 306 without conducting any chromatography on silica.

¹H NMR: 9.98 (s, 1H); 8.16 (m, 1H); 8.03 (m, 1H); 7.77 (m, 2H); 7.65 (d; 2H); 7.08 (m, 1H); 7.01 (m, 1H); 6.81 (m, 2H); 6.53 (m, 1H); 4.59 (m, 1H); 3.75 (m, 1H); 3.67 (m, 3H); 2.72 (m, 2H); 2.22 (m, 4H); 1.97 (m, 2H); 1.74 (m, 2H); 1.44 (m, 2H); 1.30 (m, 2H); 1.10 (m, 6H); 0.88 (m, 3H).

MS (APCI⁺): 607 (M+H)⁺

Elemental analysis: found C, 63.83. H, 7.02. N, 8.04. calculated for C₃₄H₄₃FN₄O₅.2H₂O C, 63.53. H, 7.375. N, 8.72

### 4-(1-Butyl-piperidin-4-yloxy)-N-(4-{2-ethoxy-4-[3-(1-ethyl-propyl)-ureido]-5-fluoro-phenoxy}-phenyl)-2-fluoro-5-methyl-benzamide

### Example 309

The 4-(1-Butyl-piperidin-4-yloxy)-2-fluoro-5-methyl-benzoic acid is reacted with the 1-[4-(4-Amino-phenoxy)-5-ethoxy-2-fluoro-phenyl]-3-(1-ethyl-propyl)-urea. The desired product is isolated in TFA salt form, after purification of the reaction medium by chromatography on silica eluting with a DCM/MeOH/NH₄OH mixture (95:5:0.5 v/v/v), followed by semi-preparative HPLC.

### (1-Ethyl-propyl)-carbamate of 4-{4-[4-(1-butyl-piperidin-4-yloxy)-3-methyl-benzoylamino]-phenoxy}-3-methoxy-phenyl

### Example 310

The 4-(1-Butyl-piperidin-4-yloxy)-3-methyl-benzoic acid is reacted with (1-Ethyl-propyl)-carbamate of 4-(4-amino-phenoxy)-3-methoxy-phenyl following the operating mode described in Example 306 using 1.5 eq of acid and 1 eq of amine. The desired product is isolated in hydrochloride form after purification of the reaction medium by chromatography on silica eluting with a DCM/MeOH/NH₄OH mixture (95:5:0.5 v/v/v), followed by treatment with a HCl/diethyl ether mixture and washing of the precipitate thus obtained in diethyl ether.

### 4-(1-Butyl-piperidin-4-yloxy)-N-(4-{5-fluoro-2-methoxy-4-[3-(1-methoxymethyl-propyl)-ureido]-phenoxy}-phenyl)-3-methyl-benzamide

### Example 311

4-(1-Butyl-piperidin-4-yloxy)-3-méehyl-benzoic acid is reacted with 1-[4-(4-Amino-phenoxy)-2-fluoro-5-methoxy-phenyl]-3-(1-methoxymethyl-propyl)-urea following the operating mode described in Example 303 using 1.2 eq of acid and 1 eq of amine. The desired product is isolated in free base form after washing the reaction medium with water, with a dilute sodium hydroxide solution, with water, with a 1 N aqueous HCl solution, and drying the organic layer over MgSO₄, filtering and concentrating the filtrate.

### 4-(1-Butyl-piperidin-4-yloxy)-N-(4-{4-[3-(1-ethyl-propyl)-ureido]-5-fluoro-2-methoxy-phenoxy}-phenyl)-3-methyl-benzamide

### Example 312

Method 1: The desired product is obtained from 4-(1-Butyl-piperidin-4-yloxy)-3-methyl-benzoic acid and 1-[4-(4-Amino-phenoxy)-2-fluoro-5-methoxy-phenyl]-3-(1-ethyl-propyl)-urea following the operating mode described in Example 306, leaving the amine to react 2.5 h at AT.
Method 2: Following General Procedure L1, the desired product is obtained from 4-(1-Butyl-piperidin-4-yloxy)-3-methyl-benzoic acid and 1-[4-(4-Amino-phenoxy)-2-fluoro-5-methoxy-phenyl]-3-(1-ethyl-propyl)-urea. The desired product is isolated in hydrochloride form after chromatography on silica eluting with a DCM/MeOH/NH₄OH mixture (95:5:0.5 v/v/v), followed by treatment with a HCl/diethyl ether mixture and washing of the precipitate thus formed in ethyl acetate.

¹H NMR: 10.00 (m, 2H); 8.25 (s, 1H); 8.09 (d, 1H); 7.8 (m, 2H); 7.65 (d, 2H); 7.2-7.1 (m, 1H); 7.00 (d, 2H); 6.8 (d, 2H); 6.5 (d, 1H); 4.9 (m, 0.6H); 4.7 (m, 0.4H); 3.67 (s, 3H); 3.6-3.52 (m, 1H); 3.5-3.4 (m, 1H); 3.18-3.00 (m, 2H); 2.35-2.05 (m, 6H); 1.9 (m, 1H); 1.72-1.61 (m, 2H); 1.52-1.42 (m, 2H); 1.29-1.4 (m, 4H); 0.92 (t, 3H); 0.87 (t, 6H).

MS (APCI⁺): 635 (M+H)⁺

Elemental analysis: found C, 62.16. H, 7.07. N, 8.35. calculated for C₃₆H₄₇FN₄O₅. 1HCl.1,2H₂O C, 62.41. H, 7.33. N, 8.09

Method 3: 4-(1-Butyl-piperidin-4-yloxy)-3-methyl-benzoic acid is reacted with 1-[4-(4-Amino-phenoxy)-2-fluoro-5-methoxy-phenyl]-3-(1-ethyl-propyl)-urea following the operating mode described in Example 306, leaving the amine to act for 2.5 h at AT. The product is purified to free base form by chromatography on silica eluting with a DCM/MeOH/NH₄OH mixture (95:5:0.5 v/v/v). The product obtained is redissolved in acetone warming the suspension to 40° C. Then maleic acid (1.1 eq) is added. After leaving the homogeneous solution obtained to stand for 36 h, the formed crystals are filtered. This yields the product in maleate form.

MS (APCI⁺): 635 (M+H)⁺

Elemental analysis: found C, 62.40. H, 6.68. N, 7.20. calculated for C₃₆H₄₇FN₄O₅.1C₄H₄H₄.1H₂O C, 62.49. H, 6.95. N, 7.29

### N-{4-[5-Fluoro-4-(3-isopropyl-ureido)-2-methoxy-phenoxy]-phenyl}-4-[1-(3-methoxy-propyl)-piperidin-4-yloxy]-3-methyl-benzamide

### Example 313

4-[1-(3-Methoxy-propyl)-piperidin-4-yloxy]-3-methyl-benzoic acid is reacted with 1-[4-(4-Amino-phenoxy)-2-fluoro-5-methoxy-phenyl]-3-isopropyl-urea following the operating mode described in Example 306, for 5 h at AT. The desired product is isolated in hydrochloride form after purifying the reaction medium by chromatography on silica, eluting with a DCM/MeOH/NH₄OH mixture (9:1:0.5 v/v/v), followed by treatment with a HCl/diethyl ether mixture and washing of the precipitate thus obtained in diethyl ether.

¹H NMR: 10.8 (m, 1H); 10.11 (s, 1H); 8.30 (m, 1H); 8.09 (d, 1H); 7.88 (m, 2H); 7.73 (d; 2H); 7.23-7.17 (m, 2H); 7.06 (m, 1H); 6.88 (d, 2H); 6.71 (m, 1H); 4.98 (m, 0.6H); 4.72 (m, 0.4H); 3.85-3.79 (m; 1H); 3.74 (s, 3H); 3.61 (m, 1H); 3.47-3.42 (m, 3H); 3.31 (s, 3H); 3.20-3.09 (m, 4H); 2.36-2.27 (m, 5H); 2.15-2.00 (m, 4H); 1.13 (d, 6H).

MS (APCI⁺): 623 (M+H)⁺

Elemental analysis: found C, 60.58. H, 6.78. N, 8.09. calculated for C₃₄H₄₃FN₄O₆.1HCl.1H₂O C, 60.30. H, 6.85. N, 8.27

### Example 314

### 4-(4-Butyl-[1,4]diazepan-1-yl)-N-(4-{2-ethoxy-4-[3-(1-ethyl-propyl)-ureido]-phenoxy}-phenyl)-2,5-difluoro-benzamide

205 mg of 4-(4-Butyl-[1,4]diazepan-1-yl)-2,5-difluoro-benzoic acid are heated for 1 h at 40° C. with 2 mL of oxalyl chloride in 5 mL DCM, and stirred 2 days at AT. The reaction medium is evaporated in vacuo. To the acid chloride thus formed is added 4 mL THF, DIEA (2.7 eq) and 1-[4-(4-Amino-phenoxy)-3-ethoxy-phenyl]-3-(1-ethyl-propyl)-urea (1 eq), stirred 24 h at AT then concentrated to dryness. The residue is redissolved in DCM, washed with a dilute aqueous K₂CO₃ solution, the organic layer is dried over MgSO₄, filtered and the filtrate concentrated. The desired product is isolated in free base form after chromatography on silica eluting with a DCM/MeOH/NH₄OH mixture 90:10:0.1 v/v/v).

### Example 315

### 4-(1-Butyl-piperidin-4-yloxy)-N-(4-{4-[3-(1-ethyl-propyl)-ureido]-2-methoxy-phenoxy}-phenyl)-2,5-difluoro-benzamide

102 mg of 4-(1-Butyl-piperidin-4-yloxy)-2,5-difluoro-benzoic acid are heated 1 h at 55° C. with avec 2 mL of oxalyl chloride. The reaction medium is evaporated in vacuo. The acid chloride thus formed is reacted in 2 mL THF with DIEA (2 eq) and 1-[4-(4-Amino-phenoxy)-3-methoxy-phenyl]-3-(1-ethyl-propyl)-urea (1 eq), and stirred 48 h at AT, then concentrated to dryness. The desired product is isolated in hydrochloride form after chromatography on silica eluting with a DCM/MeOH/NH₄OH mixture (98:2:0.1v/v/v), followed by treatment with a HCl/diethyl ether mixture.

### Example 316

### 4-[1-(2-Ethoxy-ethyl)-piperidin-4-yloxy]-N-(4-{4-[3-(1-ethyl-propyl)-ureido]-5-fluoro-2-methoxy-phenoxy}-phenyl)-3-methyl-benzamide

### A/ 4-(1-Benzyl-piperidin-4-yloxy)-3-methyl-benzonitrile

To a suspension of NaH (1.5 eq) in DMF (80 mL) is added 1-benzyl-piperidin-4-ol (15 g), stirred at AT for 45 min, then heated at 50° C. for 2 h. Next, 4-chloro-3-methylbenzonitrile (1 eq) is added and heated 12 h at 50° C. The solvent is evaporated in vacuo, the residue redissolved in an aqueous 1 N HCl solution, the aqueous layer is washed with TBME, the precipitate formed is filtered and washed with MeOH. 11.7 g of desired product are obtained.

### B/ 4-(1-Benzyl-piperidin-4-yloxy)-3-methyl-benzoic acid

Following General Procedure B, 11.8 g of desired product are isolated by treating the compound obtained in the preceding step.

### C/ 4-(1-Benzyl-piperidin-4-yloxy)-N-(4-{4-[3-(1-ethyl-propyl)-ureido]-5-fluoro-2-methoxy-phenoxy}-phenyl)-3-methyl-benzamide

1.09 g of compound of the preceding step in 20 ml thionyl chloride are heated under reflux for 1 h. The reaction medium is evaporated in vacuo. The acid chloride thus formed is reacted in 100 mL DCM with 1.09 g of the compound obtained such as described under Preparation 154, in the presence of 2 eq TEA. The reaction medium is diluted with an aqueous 1 N HCl solution, the gum formed is isolated, washed with water, redissolved in acetone and the solvent evaporated. The solid obtained is recrystallized in 6 mL of hot isopropanol, filtered, washed with cold isopropanol. 1.6 g of desired product are obtained.

### D/ N-(4-{4-[3-(1-Ethyl-propyl)-ureido]-5-fluoro-2-methoxy-phenoxy}-phenyl)-3-methyl-4-(piperidin-4-yloxy)-benzamide

The product obtained in the preceding step in solution in ethanol is treated with hydrogen under AP and at AT in the presence of a catalytic quantity of 10% palladium on charcoal. The catalyst is filtered and the solvent evaporated in vacuo. 1.39 g of desired product are obtained, which is used as such.

### E/ 4-[1-(2-Ethoxy-ethyl)-piperidin-4-yloxy]-N-(4-{4-[3-(1-ethyl-propyl)-ureido]-5-fluoro-2-methoxy-phenoxy}-phenyl)-3-methyl-benzamide

A mixture of 367 mg of amine obtained in the preceding step, of DIEA (3 eq) and of 2-bromoethylethylether (2 eq) in 8 mL DMF is heated 6 h at 80° C. After evaporation in vacuo, the residue is redissolved in DM, washed with an aqueous 1 N HCl solution, the organic layer is dried over MgSO₄, filtered and the filtrate concentrated. The desired product is obtained in hydrochloride form after precipitation in diethyl ether.

Following the same operating mode as described in Example 315, the following compounds are obtained:

### N-(4-{4-[3-(1-Ethyl-propyl)-ureido]-5-fluoro-2-methoxy-phenoxy}-phenyl)-4-[1-(2-methoxy-ethyl)-piperidin-4-yloxy]-3-methyl-benzamide

### Example 317

### N-(4-{4-[3-(1-Ethyl-propyl)-ureido]-5-fluoro-2-methoxy-phenoxy}-phenyl)-3-methyl-4-(piperidin-4-yloxy)-benzamide is reacted with 2-bromoethylmethylether. After evaporation in vacuo, the residue is redissolved in DCM, washed with a dilute aqueous K₂CO₃ solution, the organic layer is dried over MgSO₄, filtered and the filtrate concentrated. The desired product is obtained in hydrochloride form after flash chromatography on silica, eluting with a DCM/MeOH/NH₄OH mixture (95:5:0.5 v/v/v), followed by treatment with a HCl/diethyl ether mixture.

### N-(4-{4-[3-(1-Ethyl-propyl)-ureido]-5-fluoro-2-methoxy-phenoxy}-phenyl)-4-[1-(3-methoxy-propyl)-piperidin-4-yloxy]-3-methyl-benzamide

### Example 318

### A/ 3-Methoxy-propan-1-ol

9.3 g of sodium are gradually added to 95 mL of propanediol, stirred 1 h at AT, then methyl iodide (25.6 g) is added gradually and stirred 24 h at AT. 24 g of desired product are obtained by distilling (AP, 134° C.)

### B/ 1-Bromo-3-methoxy-1-propane

11.2 mL of PBr₃ in a solution of the compound obtained in the preceding step are gradually added to 4.3 mL of pyridine keeping the temperature of the reaction medium to below 60° C. The solution is stirred 30 min at 60° C., the reaction medium is poured into water, stirred 15 h at AT, extracted with DCM, and the organic layer is dried over MgSO₄, filtered and the filtrate concentrated. 10.6 g of desired product are obtained by distillation (AP, 108-122° C.).

### C/ N-(4-{4-[3-(1-Ethyl-propyl)-ureido]-5-fluoro-2-methoxy-phenoxy}-phenyl)-4-[1-(3-methoxy-propyl)-piperidin-4-yloxy]-3-methyl-benzamide

N-(4-{4-[3-(1-Ethyl-propyl)-ureido]-5-fluoro-2-methoxy-phenoxy}-phenyl)-3-methyl-4-(piperidin-4-yloxy)-benzamide is reacted with 1-bromo-3-methoxy-propane. After evaporation in vacuo, the residue is redissolved in DCM, washed with water, with an aqueous 1 N HCl solution, with an aqueous 1 N sodium hydroxide solution, with water, the precipitate formed is filtered and the organic layer is dried over MgSO₄, filtered and the filtrate concentrated. The precipitate as well as the residue from the organic layer are purified by flash chromatography on silica, eluting with a DCM/MeOH/NH₄OH mixture (92:8:0.5 v/v/v), followed by treatment with a HCl/diethyl ether mixture.

¹H NMR: 10.28 (s, 1H); 10.01 (s, 1H); 8.25 (s, 1H); 8.08 (d, 1H); 7.82 (m, 2H); 7.66 (d; 2H); 7.28-7.10 (m, 1H); 7.00 (d, 1H); 6.82 (d, 2H); 6.51 (d, 1H); 4.9 (m, 0.6H); 4.68 (m, 0.4H); 3.62-3.55 (m, 1H); 3.54-3.4 (m, 2H); 3.40 (m, 3H); 3.25 (s, 3H); 3.2-3.00 (m, 4H); 2.29-2.05 (m, 6H); 1.89-1.98 (m, 3H); 1.47 (m, 2H); 1.37 (m, 2H); 0.86 (t, 6H).

MS (APCI⁺): 651 (M+H)⁺

Elemental analysis: found C, 61.35. H, 6.99. N, 7.77. calculated for C₃₆H₄₇FN₄O₆.1HCl.1H₂O C, 61.31. H, 7.15. N, 7.94

### Example 319

### 4-(1-Butyl-piperidin-4-amino)-N-(4-{2-ethoxy-4-[3-(1-ethyl-propyl)-ureido]-phenoxy}-phenyl)-benzamide

### A/ 4-(1-Benzyl-piperidin-4-ylamino)-benzonitrile

A solution of 4-amino-N-benzylpiperidine (5 g), of 4-fluorobenzonitrile (1.3 eq) and of TEA (16 mL) in 65 mL DMSO is heated 5 h at 150° C. Then the reaction medium is poured into ice water, the precipitate filtered, washed with diisopropyl ether and dried. 1.5 g of desired product are obtained.

### B/ 4-(1-Benzyl-piperidin-4-ylamino)-benzoic acid

Following General Procedure B, 992 mg of desired product are isolated by treating the compound obtained in the preceding step.

### C/ 4-(1-Benzyl-piperidin-4-ylamino)-benzotriazol-1-yl benzoate

A mixture of the compound of the preceding step, of TBTU (1.33 g), of HOBT (0.560 g) and of DIEA (2.11 mL) in 65 mL DCM is stirred 1 h at AT, the reaction medium is washed with water, then with an aqueous 0.1 N NaOH solution, then water, the organic layer is dried over MgSO₄, filtered and the solvent evaporated in vacuo at 60° C. The desired product is obtained, which is used as such.

### D/ 4-(1-Benzyl-piperidin-4-ylamino)-N-(4-{2-ethoxy-4-[3-(1-ethyl-propyl)-ureido]-phenoxy}-phenyl)-benzamide

A solution of the compound of the preceding step, 995 mg of compound obtained such as described under Preparation 71 and 425 μL of DIEA in 3.5 mL DMF is stirred 12 h at AT. The solvent is evaporated in vacuo, the residue redissolved in water, the precipitate filtered, washed, with water and with pentane. After flash chromatography on silica eluting with a DCM/MeOH/NH₄OH mixture (95:5:0.5 v/vv), 974 mg of desired product are obtained.

### E/ N-(4-{2-Ethoxy-4-[3-(1-ethyl-propyl)-ureido]-phenoxy}-phenyl)-4-(piperidin-4-ylamino)-benzamide

The product obtained in the preceding step in solution in ethanol is treated with hydrogen under AP and at AT in the presence of a catalytic quantity of Pd(OH)₂. The catalyst is filtered and the solvent evaporated in vacuo. 70 mg of desired product are obtained, which is used as such.

### F/ 4-(1-Butyl-piperidin-4-ylamino)-N-(4-{2-ethoxy-4-[3-(1-ethyl-propyl)-ureido]-phenoxy}-phenyl)-benzamide

The compound of the preceding step, 3 eq of K₂CO₃ and 1.2 eq of 1-bromobutyl in 3 mL DMF are heated 7 h at 95° C. 1 mL of water is added followed by evaporation in vacuo. The desired product is isolated in free base form, after flash chromatography on silica eluting with a DCM/MeOH/NH₄OH mixture (95:5:0.5 v/v/v). N-(4-{2-Ethoxy-4-[3-(1-ethyl-propyl)-ureido]-phenoxy}-phenyl)-4-(1-ethyl-piperidin-4-ylamino)-benzamide is also isolated, corresponding to Example 320, in the form of a free base.

### Example 321

### N-(2-Dimethylamino-ethyl)-N-(4-{4-[3-(1-ethyl-propyl)-ureido]-2-methoxy-phenoxy}-phenyl)-4-(1-methyl-piperidin-4-yloxy)-benzamide

### A/ 4-(4-{2-Dimethylamino-ethyl)-[4-(2-methoxy-4-nitro-phenoxy)-phenyl]-carbamoyl}-phenoxy)-piperidine-1-tertbutyl carboxylate

A suspension of NaH (4 eq), of compound obtained such as described under step D of Preparation 122 (3.78 mmol) and of (2-Chloro-ethyl)-dimethyl-amine hydrochloride (2 eq) in DMSO (40 mL) is stirred 12 h at AT. The reaction medium is poured into water, extracted with TBME and with ethyl acetate, the organic layers are dried over MgSO₄, filtered and the filtrate concentrated to dryness. After chromatography on silica eluting with a DCM/MeOH mixture (95:5 v/v), 239 mg of desired product are obtained.

### B/ 4-{4-[[4-(4-Amino-2-methoxy-phenoxy)-phenyl]-(2-dimethylamino-ethyl)-carbamoyl]-phenoxy}-piperidine-1-tertbutylcarboxylate

By treating 920 mg of compound obtained such as described in the preceding step, following General Procedure E, 852 mg of desired product are obtained, which is used as such.

### C/ 4-{4-[(2-Dimethylamino-ethyl)-(4-{4-[3-(1-ethyl-propyl)-ureido]-2-methoxy-phenoxy}-phenyl)-carbamoyl]-phenoxy}-piperidine-1-tertbutyl carboxylate

The compound of the preceding step is treated following General Procedure H. After chromatography on silica eluting with a DCM/MeOH/NH₄OH mixture (95:5:0.1 v/v/v), 570 mg of desired product are obtained.

### D/ N-(2-Dimethylamino-ethyl)-N-(4-{4-[3-(1-ethyl-propyl)-ureido]-2-methoxy-phenoxy}-phenyl)-4-(piperidin-4-yloxy)-benzamide

570 mg of desired product are obtained by treating the compound of the preceding step following General Procedure C.

### E/ N-(2-Dimethylamino-ethyl)-N-(4-{4-[3-(1-ethyl-propyl)-ureido]-2-methoxy-phenoxy}-phenyl)-4-(1-methyl-pipéridin-4-yloxy)-benzamide

A mixture of compound obtained such as described in the preceding step (200 mg) and of an aqueous 37% formaldehyde solution (1 eq) in 1.68 mL of chloroform is stirred 1 h at AT. Then sodium triacetoxyborohydride (3 eq) is added and heated under reflux 48 h. The salts are filtered and the desired product is obtained in hydrochloride form after purification of the reaction medium by semi-preparative HPLC, followed by treatment with a HCl/diethyl ether mixture as per the operating mode described in Example 19.

### Example 322

### N-(4-{4-[3-(1-Ethyl-propyl)-ureido]-2-methoxy-phenoxy}-phenyl)-4-{1-[3-(tetrahydro-pyran-4-ylamino)-propyl]-piperidin-4-yloxy}-benzamide

A solution of N-(4-{4-[3-(1-Ethyl-propyl)-ureido]-2-methoxy-phenoxy}-phenyl)-4-(piperidin-4-yloxy)-benzamide (775 mg), of DIEA (2 eq), of tetrahydro-4H-pyran-4-one (1 eq) in 30 mL DCM and 15 mL acetonitrile is heated at 50° C. for 1.5 h. Sodium triacetoxyborohydride is added (1.5 eq), and stirred for 12 h at AT, then 5 mL of a saturated NaHCO₃ solution are added and the reaction medium concentrated to dryness. The desired product is obtained in hydrochloride form after flash chromatography on silica eluting with a DCM/MeOH/NH₄OH mixture (80:20:0.5 v/v/v), followed by treatment with a HCl/diethyl ether mixture.

### Example 323

### 4-[(1-Butyl-piperidin-4-yl)-methyl-amino]-N-(4-{2-ethoxy-4-[3-(1-ethyl-propyl)-ureido]-phenoxy}-phenyl)-benzamide

### A/ 4-[(1-Benzyl-piperidin-4-yl)-methyl-amino]-N-(4-{2-ethoxy-4-[3-(1-ethyl-propyl)-ureido]-phenoxy}-phenyl)-benzamide

A solution of compound obtained such as described under step D of Example 319 (500 mg) and of formaldehyde (1 eq) in DCM (3 mL) is stirred 12 h at AT. Sodium cyanoborohydride (2 eq) is then added and stirred 12 h at AT. The reaction-medium is diluted with DCM, washed with an aqueous 1 N sodium hydroxide solution then with an aqueous 1 N HCl solution, the aqueous layer is dried over MgSO₄, filtered and the filtrate concentrated. 380 mg of desired product are obtained.

### B/ N-(4-{2-Ethoxy-4-[3-(1-ethyl-propyl)-ureido]-phenoxy}-phenyl)-4-(methyl-piperidin-4-yl-amino)-benzamide

The product obtained in the preceding step in solution in ethanol is treated with hydrogen under AP and at AT in the presence of a catalytic quantity of Pd(OH)₂. The catalyst is filtered and the solvent evaporated in vacuo. 180 mg of desired product are obtained, which is used as such.

### C/ 4-[(1-Butyl-piperidin-4-yl)-methyl-amino]-N-(4-{2-ethoxy-4-[3-(1-ethyl-propyl)-ureido]-phenoxy}-phenyl)-benzamide

A solution of compound obtained in the preceding step (100 mg) and of butyraldehyde (1.1 eq) in 5 mL of a DCM/CH₃CN/MeOH mixture (9:1:0.5 v/v/v) is heated at 60° C. for 1.5 h. Sodium triacetoxyborohydride (1.5 eq) is added, heating continued at 60° C. for 2.5 h, stirred 12 h at AT, then the reaction medium is concentrated to dryness. The desired product is obtained in hydrochloride form after flash chromatography on silica eluting with a DCM/MeOH/NH₄OH mixture (80:20:0.5 v/v/v), followed by treatment with a HCl/diethyl ether mixture.

### Example 324

### 4-(1-Butyl-piperidin-4-yloxy)-N-(4-{4-[3-(1-ethyl-propyl)-ureido]-3-fluoro-phenoxy}-3-methyl-phenyl)-3-(2-hydroxy-ethyl)-benzamide

### A/ 4-(2-Bromo-4-cyano-phenoxy)-piperidine-1-tertbutyl carboxylate

A suspension of N—BOC-piperidinol (15 g) and NaH (1.5 eq) in DMF (80 mL) is heated 30 min at 80° C. After return to AT, 3-bromo-4-fluoro-3-benzonitrile (15 g) is added and stirred 16 h at AT. The solvent is evaporated in vacuo, the residue redissolved in water, extracted with DCM and the organic layer is dried over MgSO₄, filtered and the filtrate evaporated. After chromatography on silica eluting with a DCM/MeOH mixture (95:5 v/v), 17 g of desired product are obtained.

### B/ 4-(2-Allyl-4-cyano-phenoxy)-piperidine-1-tertbutyl carboxylate

Nitrogen is bubbled 20 min in a solution of the product obtained in the preceding step and of allyltributyl tin (17 mL) in DMF (80 mL). Then the catalyst Tetrakis-triphenylphosphine)-palladium (2.6 g) is added under nitrogen and heated 3 h at 80° C. The reaction medium is concentrated in vacuo, the residue redissolved in ethyl acetate, washed with water, and the organic layer is dried over MgSO₄, filtered and the filtrate evaporated. After chromatography on silica eluting with a cyclohexane/ethyl acetate mixture (90:10 v/v), 16 g of desired product are obtained in the form of a yellow oil.

### C/ 4-[4-Cyano-2-(2-oxo-ethyl)-phenoxy]-piperidine-1-tertbutyl carboxylate

Ozone is bubbled in a solution, at −70° C., of product obtained in the preceding step in 80 mL methanol. When the starting product has disappeared, nitrogen is bubbled and dimethylsulfide is added (5 mL) and stirred 12 h at AT. The reaction medium is concentrated in vacuo and purified by chromatography on silica eluting with a cyclohexane/ethyl acetate mixture (70:30 v/v). 11 g of desired product are obtained.

### D/ 4-[4-Cyano-2-(2-hydroxy-ethyl)-phenoxy]-piperidine-1-tertbutyl carboxylate

At 0° C., 600 mg of sodium tetraborohydride are gradually added to the compound obtained in the preceding step in solution in methanol (70 mL). The reaction medium is stirred 12 h at AT, concentrated, redissolved in DCM and washed with water. The organic layer is dried over MgSO₄, filtered and the filtrate concentrated. 8.8 g of desired product are obtained in the form of a colourless oil.

### E/ 4-[4-Carboxy-2-(2-hydroxy-ethyl)-phenoxy]-piperidine-1-tertbutyl carboxylate

Following General Procedure B, 2 g of desired product are isolated by treating the compound obtained in the preceding step.

### F/ 4-[4-(4-{4-[3-(1-Ethyl-propyl)-ureido]-3-fluoro-phenoxy}-3-methyl-phenylcarbamoyl)-2-(2-hydroxy-ethyl)-phenoxy]-piperidine-1-tertbutyl carboxylate

A solution of 85 mg of compound obtained such as described under Preparation 153, of HOBT (400 mg), EDCI (570 mg), DIEA (2 mL) and 900 mg of compound obtained in the preceding step in 10 mL DCM is heated 8 h under reflux. After return to AT, the reaction medium is washed with water, the organic layer is dried over MgSO₄ and purification conducted by chromatography on silica eluting with a DCM/MeOH/NH₄OH mixture (95:5:0.5 v/v/v). 400 mg of desired product are obtained.

### G/ N-(4-{4-[3-(1-Ethyl-propyl)-ureido]-3-fluoro-phenoxy}-3-methyl-phenyl)-3-(2-hydroxy-ethyl)-4-(piperidin-4-yloxy)-benzamide

400 mg of desired product are obtained by treating the compound of the preceding step following General Procedure C.

### H/ 4-(1-Butyl-piperidin-4-yloxy)-N-(4-{4-[3-(1-ethyl-propyl)-ureido]-3-fluoro-phenoxy}-3-methyl-phenyl)-3-(2-hydroxy-ethyl)-benzamide

A suspension of compound obtained in the preceding step (400 mg), of butyraldehyde (1 eq) and of Na₂SO₄ (500 mg) in 12 mL DCM is stirred 12 h at AT. Then sodium triacetoxyborohydride (2 eq) is added, stirred 24 h at AT, washed with water and the organic layer dried over MgSO₄, filtered and the filtrate concentrated. The desired product is obtained in TFA salt form after semi-preparative HPLC.

### Example 325

### N-(4-{4-[3-(1-Ethyl-propyl)-ureido]-2-methoxy-phenoxy}-phenyl)-N-(2-methoxy-ethyl)-4-(piperidin-4-yloxy)-benzamide

4-{4-[(4-{4-[3-(1-ethyl-propyl)-ureido]-2-methoxy-phenoxy}-phenyl)-(2-methoxy-ethyl)-carbamoyl]-phenoxy}-piperidine-1-tertbutyl carboxylate (195 mg) in 1.2 mL of a 2N HCl solution in diethyl ether and 10 mL of diethyl ether are stirred 12 h at AT, the reaction medium is evaporated, the precipitate washed with diethyl ether and with pentane. The desired product is thus obtained in hydrochloride form.

¹H NMR: 8.75 (m, 2H); 8.60 (s, 1H); 7.39 (s, 1H); 7.18 (d, 2H); 6.95 (d, 2H); 6.87-6.79 (m; 4H); 6.67 (d, 2H); 6.30 (d, 1H); 4.61 (m, 1H); 3.89 (t, 2H); 3.60 (s, 3H); 3.45 (m; 3H); 3.22 (s, 3H); 3.20 (m, 1H); 3.04 (m, 2H); 2.07-2.03 (m, 2H); 1.50-1.40 (m, 2H); 1.77-1.74 (m, 2H); 1.51-1.44 (m, 2H); 1.45-(1-30 (m, 2H); 0.85 (t, 6H)

MS (APCI⁺): 605 (M+H)⁺

Elemental analysis: found C, 62.75. H, 7.11. N, 8.60. calculated for C₃₄H₄₄N₄O₆.1,1HCl.0.4H₂O C, 62.63. H, 7.10. N, 8.59

### Example 326

### N-(4-{4-[3-(1-Ethyl-propyl)-ureido]-2,5-difluoro-phenoxy}-phenyl)-4-(piperidin-4-yloxy)-benzamide

### A/ 4-[4-(4-{4-[3-(1-Ethyl-propyl)-ureido]-2,5-difluoro-phenoxy}-phenylcarbamoyl)-phenoxy]-piperidine-1-tertbutyl carboxylate

Following General Procedure L3 to treat 1-[4-(4-Amino-phenoxy)-2,5-difluoro-phenyl]-3-(1-ethyl-propyl)-urea (209 mg) and 4-(4-Carboxy-phenoxy)-piperidine-1-tertbutyl carboxylate, 350 mg of desired product are obtained.

### B/ N-(4-{4-[3-(1-Ethyl-propyl)-ureido]-2,5-difluoro-phenoxy}-phenyl)-4-(piperidin-4-yloxy)-benzamide

The compound obtained in the preceding step is treated following General Procedure C. After free base conversion, the desired product is obtained in hydrochloride form by treating with a HCl/diethyl ether mixture.

### Example 327

### N-(4-{2-Ethoxy-4-[3-(1-ethyl-propyl)-ureido]-phenoxy}-phenyl)-4-(methyl-piperidin-4-yl-amino)-benzamide

### A/ 4-[(1-Benzyl-piperidin-4-yl)-methyl-amino]-N-(4-{2-ethoxy-4-[3-(1-ethyl-propyl)-ureido]-phenoxy}-phenyl)-benzamide

A solution of compound obtained such as described under step D in Example 319 (500 mg) and of formaldehyde (1 eq) in DCM (3 mL) is stirred 12 h at AT. Then sodium cyanoborohydride (2 eq) is added and stirred 12 h at AT. The reaction medium is diluted with DCM, washed with an aqueous 1N sodium hydroxide solution, then with an aqueous 1 N HCl solution, the organic layer is dried over MgSO₄, filtered and the filtrate concentrated. 380 mg of desired product are obtained.

### B/ N-(4-{2-Ethoxy-4-[3-(1-ethyl-propyl)-ureido]-phenoxy}-phenyl)-4-(methyl-piperidin-4-yl-amino)-benzamide

The compound obtained in the preceding step (255 mg) in solution in ethanol is treated with hydrogen under AP and AT in the presence of a catalytic quantity of Pd(OH)₂. The catalyst is filtered and the solvent evaporated in vacuo. After purification by semi-preparative HPLC in an ammonium bicarbonate medium, the compound N-(4-{2-Ethoxy-4-[3-(1-ethyl-propyl)-ureido]-phenoxy}-phenyl)-4-[(1-ethyl-piperidin-4-yl)-methyl-amino]-benzamide is obtained, corresponding to Example 328, in the form of a base as well as the desired product. The hydrochloride of the desired product is formed by treating with a HCl/diethyl ether mixture.

### Example 329

### 1-(4-{4-[4-(1-Butyl-piperidin-4-yloxy)-benzoyl]-3,4-dihydro-2H-benzo[1,4]oxazin-7-yloxy}-phenyl)-3-(1-ethyl-propyl)-urea

### A/ 2-Chloro-N-(2,4-dimethoxy-phenyl)-acetamide

Chloroacetyl chloride (8.6 mL) is gradually added to a solution of 2,4-dimethoxyaniline (15 g) and TEA (15 mL) in 15 mL DCM, keeping the temperature of the reaction medium to below 25° C. On completion of this addition the reaction medium is stirred 30 min then washed with water, with an aqueous 1N HCl solution, then with a saturated aqueous NaHCO₃ solution, and the organic layer is dried over MgSO₄, filtered and the filtrate concentrated to dryness. 20 g of desired product are obtained.

### B/ 2-Chloro-N-(2-hydroxy-4-methoxy-phenyl)-acetamide

A solution of 5 g of compound obtained in the preceding step in 50 mL DCM is cooled to 4° C. Aluminium trichloride (11.6 g) is added gradually, keeping the temperature of the reaction medium to below 10° C., followed by stirring for 1 h at 4° C. and 12 h at AT. The reaction medium is poured onto ice, extracted with ethyl acetate, the organic layer is washed with water, dried over MgSO₄, filtered and the filtrate concentrated. 4.1 g of desired product are obtained in the form of a brown powder.

### C/ 7-Methoxy-4H-benzo[1,4]oxazin-3-one

A solution of 900 mg of compound obtained in the preceding step and of K₂CO₃ (600 mg) in acetone (25 mL) is heated 3 h under reflux. The reaction medium is concentrated, the residue redissolved in DCM, washed with NaCl saturated water, the organic layer is dried over MgSO₄, filtered and the filtrate concentrated to dryness. The solid obtained is redissolved in petroleum ether, filtered, washed with diisopropyl ether and oven dried. 400 mg of desired product are obtained in the form of a brown powder.

### D/ 7-Methoxy-3,4-dihydro-2H-benzo[1,4]oxazine

A solution of compound obtained such as described in the preceding step (7 g) in THF (70 mL) is added dropwise to a suspension of LAH (3.1 g) in THF (100 mL). The mixture is heated 3 h under reflux. After return to AT, an aqueous 5% sodium hydroxide solution (30 mL) is added gradually, followed by filtering, drying the filtrate over MgSO₄, filtering and concentrating. 15 g of desired product are obtained.

### E/ 4-(1-butyl-piperidin-4-yloxy)-benzoyl chloride

A solution of 4 g of compound described under Preparation 5 and of thionyl chloride (10 mL) in DCM (100 mL) is heated 12 h under reflux. The reaction medium is concentrated to dryness, the residue redissolved in DCE and again concentrated to dryness. 4.1 g of desired product are obtained in the form of a beige powder.

### F/ [4-(1-Butyl-piperidin-4-yloxy)-phenyl]-(7-methoxy-2,3-dihydro-benzo[1,4]oxazin-4-yl)-methanone

A solution of 4.59 g of compound obtained such as described under step D, of 2.7 g of compound obtained as described under step E and of TEA (4.8 mL) in 200 mL DCM is stirred 4 days at AT. The reaction medium is washed with water, the organic layer dried over MgSO₄, filtered and the filtrate concentrated. After chromatography on silica eluting with a DCM/MeOH mixture (95:5 v/v), 6 g of desired product are obtained.

### G/ [4-(1-Butyl-piperidin-4-yloxy)-phenyl]-(7-hydroxy-2,3-dihydro-benzo[1,4]oxazin-4-yl)-methanone

At 0° C., a 1 M solution of boron tribromide in DCM (21.3 mL) and DCM (30 mL) is added dropwise to 4.9 g of product obtained in the preceding step in solution in DCM (75 mL). After stirring 12 h at AT, water (50 mL) is added gradually, decanted, the organic layer dried over MgSO₄, filtered and the filtrate concentrated. After chromatography on silica eluting with a DCM/MeOH mixture (95:5 v/v), 1.5 g of desired product are obtained.

### H/ [4-(1-Butyl-piperidin-4-yloxy)-phenyl]-[7-(4-nitro-phenoxy)-2,3-dihydro-benzo[1,4]oxazin-4-yl]-methanone

The compound obtained in the preceding step is condensed on 1-fluoro-4-nitrobenzene following General Procedure O. After chromatography on silica eluting with a DCM/MeOH mixture (95:5 v/v), 1.5 g of desired product are obtained.

### I/ [7-(4-Amino-phenoxy)-2,3-dihydro-benzo[1,4]oxazin-4-yl]-[4-(1-butyl-piperidin-4-yloxy)-phenyl]-methanone

By treating the compound obtained in the preceding step following General Procedure E, 1.34 g of desired product are obtained.

### J/ 1-(4-{4-[4-(1-Butyl-piperidin-4-yloxy)-benzoyl]-3,4-dihydro-2H-benzo[1,4]oxazin-7-yloxy}-phenyl)-3-(1-ethyl-propyl)-urea

The compound obtained in the preceding step is treated following General Procedure H. The desired product is obtained in hydrochloride form after flash chromatography on silica eluting with a DCM/MeOH/NH₄OH mixture (80:20:0.5 v/v/v), followed by treatment with a HCl/diethyl ether mixture.

### Example 330

### N-(4-{4-[3-(1-Ethyl-propyl)-ureido]-3-fluoro-phenoxy}-2-fluoro-phenyl)-4-(1-methyl-piperidin-4-yloxy)-benzamide

### A/ [2-Fluoro-4-(3-fluoro-4-nitro-phenoxy)-phenyl]-tertbutyl carbamate

A suspension of NaH (3.1 g) and of compound obtained such as described under step A of Preparation 85 (17.5 g) in DMF is stirred 30 min AT. This suspension is cooled to 0° C. and added dropwise to 8.5 ml of 2,4-difluoronitrobenzene in solution in 100 mL of DMF. The medium is stirred 3 h at AT and concentrated to dryness. The residue is redissolved in TBME and washed with water. The organic layer is dried over MgSO₄, filtered and the filtrate concentrated. The solid obtained is washed with diisopropyl ether. Chromatography on silica is performed eluting with a DCM/pentane mixture (5:5 v/v). The compound obtained is recrystallized in TBME and washed with diisopropyl ether. 2.2 g of desired product are obtained.

### B/ 2-Fluoro-4-(3-fluoro-4-nitro-phenoxy)-phenylamine

By treating the compound obtained in the preceding step following General Procedure C, 1.8 g of desired product are obtained.

### C/ 4-(1-Methyl-piperidin-4-ylamino)-benzotriazol-1-yl benzoate

A mixture of 500 mg of compound obtained such as described under Preparation 6, of TBTU (835 mg), HOBT (351 mg) and of DIEA (0.99 mL) in 40 mL DCM is stirred at AT for 30 min, the reaction medium is washed with water, with an aqueous 0.1 N NaOH solution, with water, and the organic layer is dried over MgSO₄, filtered and the solvent evaporated in vacuo. 800 mg of desired product are obtained, which is used as such.

### D/ N-[2-Fluoro-4-(3-fluoro-4-nitro-phenoxy)-phenyl]-4-(1-methyl-piperidin-4-yloxy)-benzamide

The compound of the preceding step and 500 mg of compound obtained in step B are placed in solution in 3 mL DMF at AT, the solvent is evaporated in vacuo at 60° C. and the mixture held in vacuo at 60° C. for 6 h. The reaction medium is redissolved in water, the precipitate filtered, redissolved in methanol and concentrated to dryness. After chromatography on silica eluting with a DCM/MeOH/NH₄OH mixture (95:5:0.5 v/v/v), 356 g of desired product are obtained.

### E/ N-[4-(4-Amino-3-fluoro-phenoxy)-2-fluoro-phenyl]-4-(1-methyl-piperidin-4-yloxy)-benzamide

The compound of the preceding step in 100 mL of MeOH is treated with hydrogen under AP and at AT in the presence of 100 mg of palladium on charcoal. The catalyst is filtered and the filtrate concentrated. 289 mg of desired product are obtained.

### F/ N-(4-{4-[3-(1-Ethyl-propyl)-ureido]-3-fluoro-phenoxy}-2-fluoro-phenyl)-4-(1-methyl-piperidin-4-yloxy)-benzamide

The compound obtained in the preceding step is treated following General Procedure H. The desired product is obtained in hydrochloride form after flash chromatography on silica eluting with a DCM/MeOH/NH₄OH mixture (90:10:0.5 v/v/v), followed by treatment with a HCl/diethyl ether mixture.

### Example 331

### 4-(1-Butyl-piperidin-4-yloxy)-N-(4-{4-[3-(1-ethyl-propyl)-ureido]-3-fluoro-phenoxy}-2-fluoro-phenyl)-3-methyl-benzamide

### A/ 4-(1-Butyl-piperidin-4-yloxy)-3-methyl-benzotriazol-1-yl benzoate

The desired product is obtained from 962 mg of compound obtained such as described under Preparation 4, following the method described described under step A of Example 22.

### B/ 4-(1-Butyl-piperidin-4-yloxy)-N-[2-fluoro-4-(3-fluoro-4-nitro-phenoxy)-phenyl]-3-methyl-benzamide

The compound of the preceding step and 500 mg of compound obtained at step B of Example 330 are placed in solution in 2 mL of DMF at AT, the solvent is evaporated in vacuo at 60° C. and the mixture held in vacuo at 60° C. for 12 h. The reaction medium is redissolved in DCM, washed with water, with a saturated aqueous Na₂CO₃ solution and with water. The organic layer is dried over MgSO₄, filtered and the filtrate concentrated. After chromatography on silica eluting with a DCM/MeOH/NH₄OH mixture (95:5:0.5), 480 mg of desired product are obtained.

### C/ N-[4-(4-Amino-3-fluoro-phenoxy)-2-fluoro-phenyl]-4-(1-butyl-piperidin-4-yloxy)-3-methyl-benzamide

The compound obtained in the preceding step in solution in 100 mL MeOH is treated with hydrogen under AP and at AT in the presence of 100 mg of palladium on charcoal. The catalyst is filtered and the filtrate concentrated. 430 mg of desired product are obtained.

### D/ 4-(1-Butyl-piperidin-4-yloxy)-N-(4-{4-[3-(1-ethyl-propyl)-ureido]-3-fluoro-phenoxy}-2-fluoro-phenyl)-3-methyl-benzamide

The compound obtained in the preceding step is treated following General Procedure H. The desired product is obtained in hydrochloride form after flash chromatography on silica eluting with a DCM/MeOH/NH₄OH mixture (90:10:0:5 v/v/v), followed by treatment with a HCl/diethyl ether mixture.

### Example 332

### 1-(1-Butyl-piperidin-4-yl)-1H-indole-5-carboxylic acid (4-{4-[3-(1-ethyl-propyl)-ureido]-3-fluoro-phenoxy}-2-fluoro-phenyl)-amide

### A/ 1-(1-Butyl-piperidin-4-yl)-1H-indole-5-benzotriazol-1-yl carboxylate

The desired product is obtained from 2.2 g of compound obtained such as described under Preparation 148 following the method described in step A of Example 22.

### B/ 1-(1-Butyl-piperidin-4-yl)-1H-indole-5-carboxylic acid [2-fluoro-4-(3-fluoro-4-nitro-phenoxy)-phenyl]-amide

The compound of the preceding step and 500 mg of compound obtained at step B of Example 330 are placed in solution in 2 mL DMF at AT, the solvent is evaporated in vacuo at 60° C. and the mixture held in vacuo at 60° C. for 24 h. 620 mg of desired product are isolated following the operating mode described in step B of Example 330.

### C/ 1-(1-Butyl-piperidin-4-yl)-1H-indole-5-carboxylic acid [4-(4-amino-3-fluoro-phenoxy)-2-fluoro-phenyl]-amide

496 mg of compound obtained in the preceding step in solution in 80 mL MeOH are treated with hydrogen under AP and at AT in the presence of 50 mg of palladium on charcoal. The catalyst is filtered and the filtrate concentrated. 425 mg of desired product are obtained.

### D/ 1-(1-Butyl-piperidin-4-yl)-1H-indole-5-carboxylic acid (4-{4-[3-(1-ethyl-propyl)-ureido]-3-fluoro-phenoxy}-2-fluoro-phenyl)-amide

The compound obtained in the preceding step is treated following General Procedure H. The desired product is obtained in hydrochloride form after chromatography on silica eluting with a DCM/MeOH/NH₄OH mixture (90:10:0.5 v/v/v), followed by treatment with a HCl/diethyl ether mixture.

### Example 333

### 1-(4-{9-[4-(1-Butyl-piperidin-4-yloxy)-benzoyl]-6,7,8,9-tetrahydro-5-oxa-9-aza-benzocyclohepten-3-yloxy}-3-methoxy-phenyl)-3-(1-ethyl-propyl)-urea

### A/ 5-Methoxy-2-nitro-phenol

A solution of hydroxyanisole (55 g) and acetic acid (210 mL) is added dropwise to a 68% nitric acid solution (32.9 mL) in 230 mL acetic acid keeping the temperature to below 10° C. After stirring 1 h at 10° C. and pouring onto ice, the precipitate is filtered and washed with water. After chromatography on silica eluting with DCM, 25.8 g of desired product are obtained.

### B/ 6-Methoxy-3H-benzooxazol-2-one

120 g of compound obtained in the preceding step in solution 480 mL THF are treated with hydrogen under AP and at AT in the presence of 2.5 g of 5% palladium on charcoal. At 0° C. TEA is added (23.4 mL) followed by the gradual addition of triphosgene (12 g) in solution in THF (120 mL) and stirring for 30 min at −10° C. The medium is filtered and the filtrate evaporated. After recrystallizing in toluene, 10.4 g of desired product are obtained.

### C/ 3-(4-Chloro-butyl)-6-methoxy-3H-benzooxazol-2-one

A suspension of NaH (2.7 g) and 10.3 g of compound obtained in the preceding step in DMF (30 mL) is stirred 1 h at AT. At solution is gradually added to a solution of 3-bromochloropropane (12.2 mL) in 25 mL DMF. The reaction medium is stirred 2 h at 0° C. then 12 h at AT. 20 mL of water are added to the medium, extracted with TBME, and the organic layer dried over MgSO₄, filtered and the filtrate evaporated. After chromatography on silica eluting with DCM, 9.3 g of desired product are obtained.

### D/ 3-Methoxy-6,7,8,9-tetrahydro-5-oxa-9-aza-benzocycloheptene

A solution of compound obtained in the preceding step and of KOH (10.3 g) in methoxyethanol (100 mL) is heated 48 h under reflux. The reaction medium is concentrated, redissolved in water, extracted with TBME, the organic layer is dried over MgSO₄, filtered and the filtrate concentrated. 5.6 g of desired product are obtained.

### E/ [4-(1-Butyl-piperidin-4-yloxy)-phenyl]-(3-methoxy-7,8-dihydro-6H-5-oxa-9-aza-benzocyclohepten-9-yl)-methanone

A solution of compound obtained in the preceding step (1.79 g) and of TEA (1.4 mL) in 50 mL DCM, is gradually added to a solution of compound obtained such as described in step E of Example 329 (1 eq) and TEA (1 eq) in 100 mL DCM. After stirring 24 h at AT and washing with water, with an aqueous 1 N NaOH solution, the organic layer is dried over MgSO₄, filtered and the filtrate concentrated. 2.7 g of desired product are obtained, which is used as such.

### F/ [4-(1-Butyl-piperidin-4-yloxy)-phenyl]-(3-hydroxy-7,8-dihydro-6H-5-oxa-9-aza-benzocyclohepten-9-yl)-methanone

At 0° C., a mixture containing a 1 M BBr₃ solution in DCM (27 mL) and 30 mL DCM is added dropwise to a solution of compound obtained in the preceding step and of tetrabutylammonium iodide (4.8 g) in 270 mL DCM. After stirring 12 h at AT, the medium is hydrolyzed with water, an aqueous 1 N sodium hydroxide solution is added to basic pH and the aqueous layer is washed with DCM. The aqueous layer is acidified with a concentrate HCl solution, neutralized with a saturated NaHCO₃ solution, extracted with DCM, and the organic layer is dried over MgSO₄, filtered and the filtrate concentrated to dryness. After chromatography on silica eluting with a DCM/MeOH mixture (8:2 v/v), 0.6 g of desired product are obtained.

### G/ [4-(1-Butyl-piperidin-4-yloxy)-phenyl]-[3-(2-methoxy-4-nitro-phenoxy)-7,8-dihydro-6H-5-oxa-9-aza-benzocyclohepten-9-yl]-methanone

The compound obtained in the preceding step is condensed on 4-chloronitroanisole following General Procedure O. After semi-preparative HPLC, 90 mg of desired product are obtained.

### H/ [3-(4-Amino-2-methoxy-phenoxy)-7,8-dihydro-6H-5-oxa-9-aza-benzocyclohepten-9-yl]-[4-(1-butyl-piperidin-4-yloxy)-phenyl]-methanone

By treating the compound obtained in the preceding step following General Procedure E, 87 mg of desired product are obtained.

### I/ 1-(4-{9-[4-(1-Butyl-piperidin-4-yloxy)-benzoyl]-6,7,8,9-tetrahydro-5-oxa-9-aza-benzocyclohepten-3-yloxy}-3-methoxy-phenyl)-3-(1-ethyl-propyl)-urea

The compound obtained in the preceding step is treated following General Procedure H. The desired product is obtained in TFA salt form after semi-preparative HPLC.

### Example 334

### 4-(1-Butyl-piperidin-4-yloxy)-piperidine-1-carboxylic acid (4-{2-ethoxy-4-[3-(1-ethyl-propyl)-ureido]-phenoxy}-phenyl)-amide

### A/ 4-(1-Butyl-piperidin-4-yloxy)-pyridine

Chloropyridine hydrochloride (3.4 g) is gradually added to a solution of potassium tert-butylate (5.16 g) and 1-butyl-piperidin-4-ol (3.6 g) in DMSO (11 mL). The reaction medium is stirred 3 days at AT, then poured onto ice, extracted with TBME, the organic layer is washed with water, dried over MgSO₄, filtered and the filtrate concentrated to dryness. After chromatography on silica eluting with a DCM/MeOH/NH₄OH mixture (95:5:0.5 v/v/v), 2.6 g of desired product are obtained.

### B/ 1-Butyl-4-(piperidin-4-yloxy)-piperidine

500 mg of compound obtained in the preceding step in solution in MeOH (40 mL) are treated with hydrogen in the presence of a catalytic quantity of 5% Ruthenium on charcoal, at 50 bars and 80° C. for 15 h. After filtering the catalyst, washing with MeOH and concentration of the filtrate, 230 mg of desired product are obtained.

### C/ 4-(1-Butyl-piperidin-4-yloxy)-piperidine-1-carboxylic acid (4-{2-ethoxy-4-[3-(1-ethyl-propyl)-ureido]-phenoxy}-phenyl)-amide

A solution of 1-[4-(4-Amino-phenoxy)-3-ethoxy-phenyl]-3-ethyl-propyl)-urea (370 mg) and DIEA (2.2 eq) in 10 mL DCM, is added dropwise to a solution of triphosgene (90 mg) in 10 mL DCM. After stirring 10 min at AT, a solution of compound obtained in the preceding step (230 mg) and DIEA (1.2 eq) in 10 mL DCM is added. Stirring is continued for 48 h at AT, followed by washing with water, filtering the organic layer, drying the filtrate over MgSO₄, filtering and concentrating to dryness. The desired product is obtained in hydrochloride form after flash chromatography on silica eluting with a DCM/MeOH/NH₄OH mixture (95:5:0.5 v/v/v), followed by treatment with a HCl/diethyl ether mixture.

### Example 335

### 1-(4-{4-[4-(1-Butyl-piperidin-4-yloxy)-phenoxymethyl]-phenoxy}-3-methoxy-phenyl)-3-(1-ethyl-propyl)-urea

### A/ 4-toluene-4-sulfonyloxy)-piperidine-1-tertbutyl carbamate

11.5 g of para-toluenesulfonyl chloride are added to 10 g of N—BOC-4-hydroxypiperidine in 40 mL pyridine, stirred 12 h at AT, poured into 200 mL water, the precipitate filtered, the precipitate washed with water. The solid obtained is redissolved in DCM, washed with water, and the organic layer concentrated. The residue is washed with pentane, the precipitate filtered. 12.7 g of desired product are obtained in the form of a white powder.

### B/ 4-(4-Benzyloxy-phenoxy)-piperidine-1-tertbutyl carbamate

A solution of benzyloxyphenol (4 g) and of KOH (1.1 g) in 200 mL ethanol is heated 1 h under reflux. 7 g of compound obtained in the preceding step are added and heating under reflux continued for 10 h. After return to AT, evaporation, the residue is redissolved in DCM, washed with 1N sodium hydroxide, the organic layer dried over MgSO₄, filtered and the filtrate concentrated. The residue is redissolved in pentane and 2.4 g of desired product are obtained in the form of a white solid.

### C/ 4-(4-Hydroxy-phenoxy)-piperidine-1-tertbutyl carbamate

2.4 g of compound obtained in the preceding step in solution in 50 mL ethanol are treated with hydrogen under a pressure of 5 bars at AT in the presence of 10% palladium on charcoal and acetic acid (2 mL). The catalyst is filtered and the filtrate concentrated. The residue is redissolved in DCM, dried over MgSO₄, filtered and the filtrate concentrated. 1.5 g of desired product are obtained.

### D/ 2-Methoxy-4-nitro-1-p-tolyloxy-benzene

A solution of 2-chloro-5-nitroanisole (5 g), para-cresol (2.9 g) and of K₂CO₃ (3.5 g) in 200 mL DMF is heated 8 h under reflux. The solvent is evaporated, the residue redissolved in water, extracted with ethyl acetate, and the organic layer is washed with water and sodium hydroxide. The organic layer is dried over MgSO₄, filtered and the filtrate concentrated. 5.6 g of desired product are obtained in the form of an ochre powder.

### E/ 1-(4-Bromomethyl-phenoxy)-2-methoxy-4-nitro-benzene

A solution of compound obtained in the preceding step (4.6 g), of N-bromosuccinimide (3.2 g) and of AIBN (20 mg) in 60 mL DCE is heated 5 h under reflux. After return to AT, the reaction medium is washed with water, the organic layer dried over MgSO₄, filtered and the filtrate concentrated. The residue is redissolved in diisopropyl ether and filtered. 1.7 g of desired product are obtained in the form of a cream-coloured solid.

### F/ 4-{4-[4-(2-Methoxy-4-nitro-phenoxy)-benzyloxy]phenoxy}-piperidine-1-tertbutyl carbamate

A solution of compound obtained in step E (1.7 g), of compound obtained in step C and of K₂CO₃ (700 mg) in 100 mL methylethylcetone is heated under reflux for 7 h. The reaction medium is concentrated, the residue redissolved in DCM, washed with water and the organic layer is dried over MgSO₄, and the filtrate concentrated. The residue is redissolved in diethyl ether, the precipitate filtered and washed with water. 1.6 g of desired product are obtained in the form of a white solid.

### G/ 4-{4-[4-(2-Methoxy-4-nitro-phenoxy)-benzyloxy]-phenoxy}-piperidine

At 0° C., a 3N solution of HCl in diethyl ether is added to a solution of the compound obtained in the preceding step (1 g), stirred 6 h at AT, then the solvent is evaporated, the residue redissolved in an acetone/ether mixture (1:1 v/v), and the precipitate filtered. 820 mg of desired product are obtained.

### H/ 1-Butyl-4-{4-[4-(2-methoxy-4-nitro-phenoxy)-benzyloxy]-phenoxyl}-piperidine

A suspension of compound obtained in the preceding step (820 mg), of butyraldehyde (1.2 eq), of DIEA (1 eq) and of sodium triacetoxyborohydride (2 eq) in 15 mL DCM is stirred 12 h at AT. The reaction medium is then washed with water, with a saturated K₂CO₃ solution, and the organic layer is dried over MgSO₄, filtered and the filtrate concentrated. The residue is redissolved in pentane and the precipitate filtered. 700 mg of desired product are obtained.

### G/ 4-{4-[4-(1-Butyl-piperidin-4-yloxy)-phenoxymethyl]-phenoxy}-3-methoxy-phenylamine

600 mg of compound obtained in the preceding step in solution in 25 mL of a methanol/THF mixture (1:1 v/v) are treated with hydrogen under AP and at AT, in the presence of platinum oxide. The catalyst is filtered and the filtrate concentrated. 300 mg of desired product are obtained.

### H/ 1-(4-{4-[4-(1-Butyl-piperidin-4-yloxy)-phenoxymethyl]-phenoxy}-3-methoxy-phenyl)-3-(1-ethyl-propyl)-urea

The compound obtained in the preceding step is treated following General Procedure H. The desired product is obtained in hydrochloride form after flash chromatography on silica eluting with a DCM/MeOH/NH₄OH mixture (98:2:0.2 v/v/v), followed by treatment with a HCl/diethyl ether mixture.

The structures of the compounds of the invention thus synthesized are presented below with their expected mass and observed mass after mass spectrometry.

| | | (M + H)⁺ | (M + H)⁺ |
|---|---|---|---|
| | | observed | expected |
| | | HPLC/MS | HPLC/MS |
| Example no. | Structure | APCI⁺ | APCI⁺ |
| 1 | 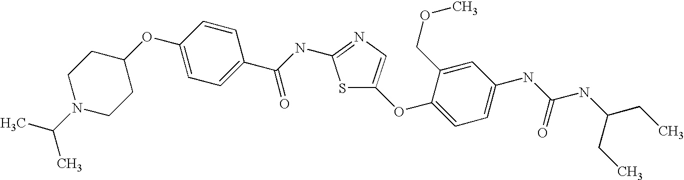 | 611 | 611 |
| 2 | 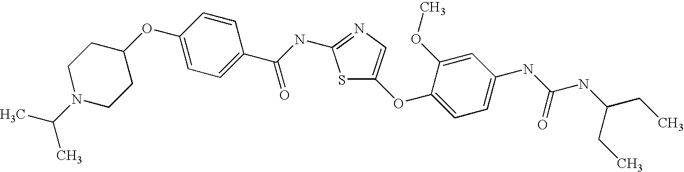 | 597 | 597 |
| 3 | 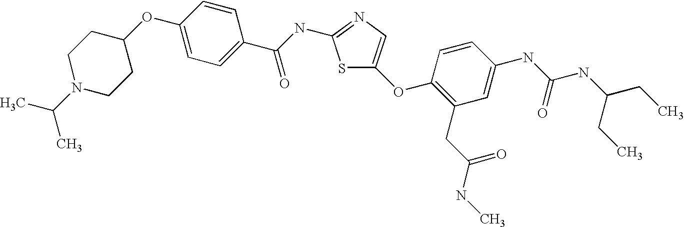 | 638 | 638 |
| 4 | 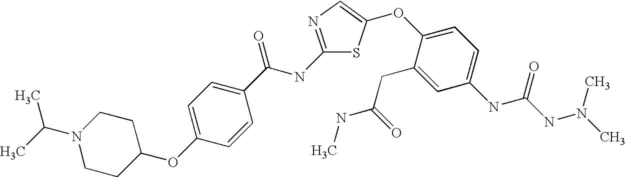 | 611 | 611 |
| 5 | 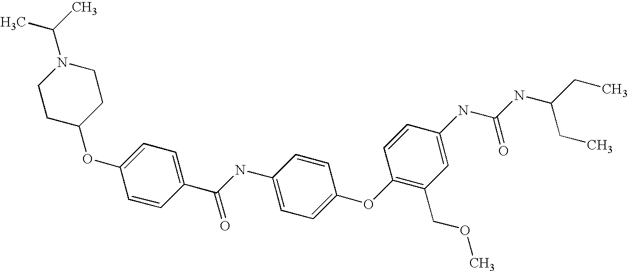 | 604 | 604 |
| 6 | 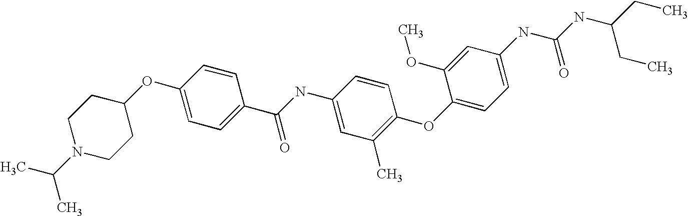 | 604 | 604 |
| 7 | 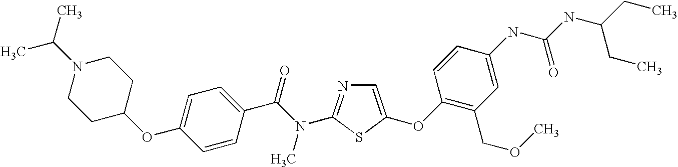 | 625 | 625 |
| 8 | 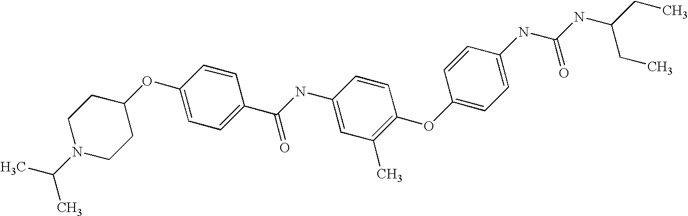 | 574 | 574 |
| 9 | 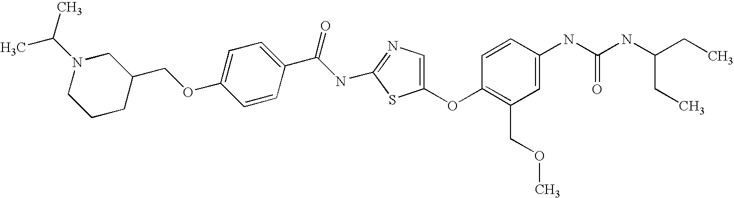 | 625 | 625 |
| 10 | 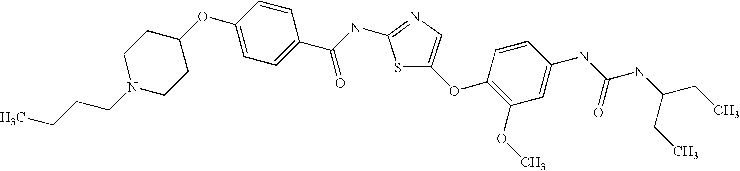 | 611 | 611 |
| 11 | 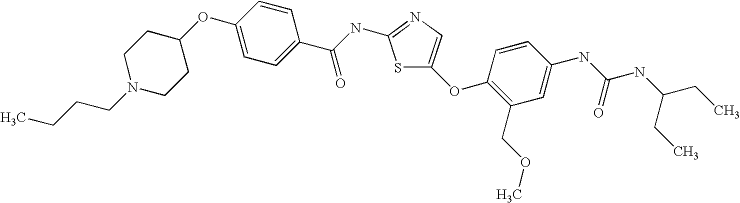 | 625 | 625 |
| 12 | 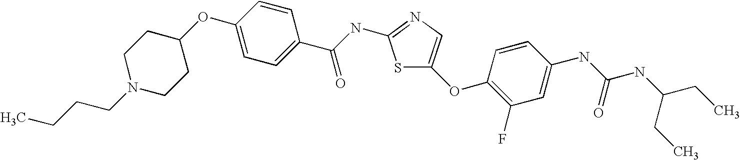 | 599 | 599 |
| 13 | 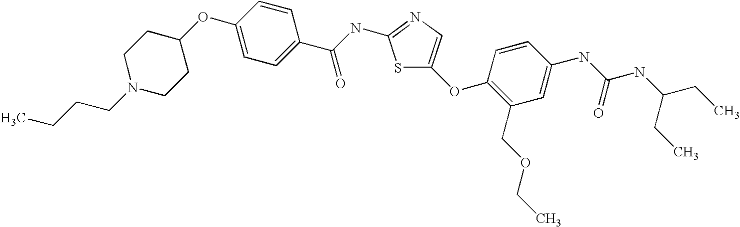 | 639 | 639 |
| 14 | 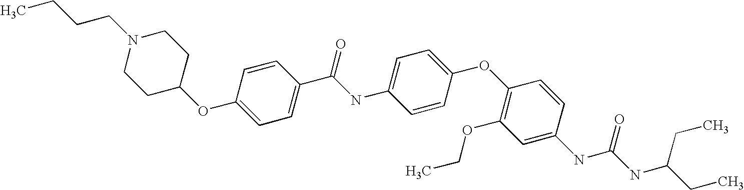 | 618 | 618 |
| 15 | 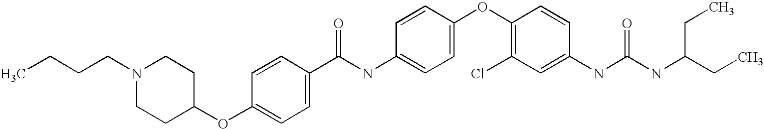 | 608 | 608 |
| 16 | 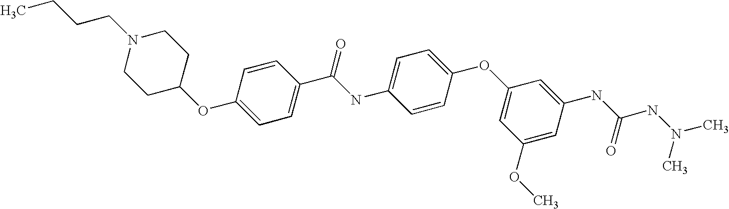 | 577 | 577 |
| 17 | 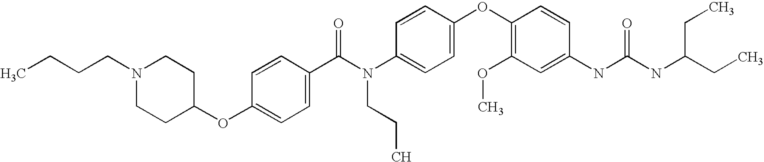 | 647 | 647 |
| 18 | 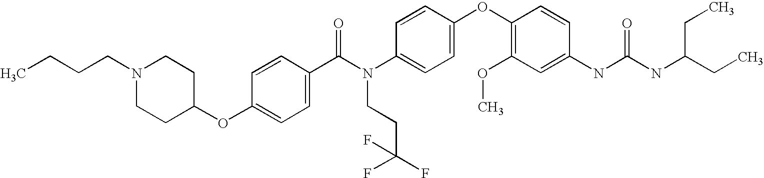 | 699 | 699 |
| 19 | 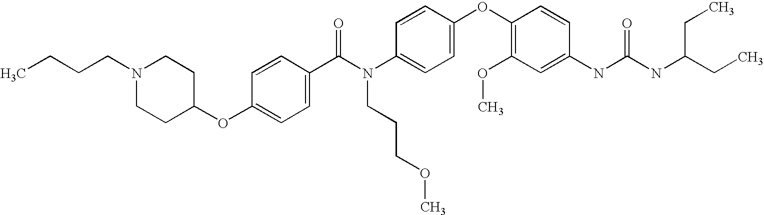 | 675 | 675 |
| 20 | 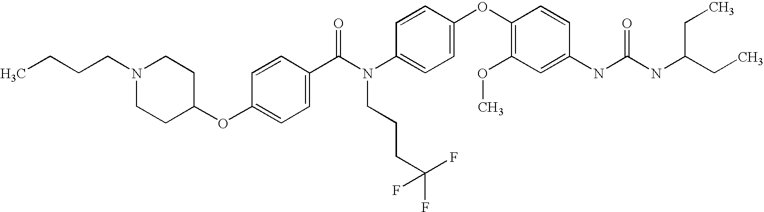 | 713 | 713 ^{(a)} |
| 21 | 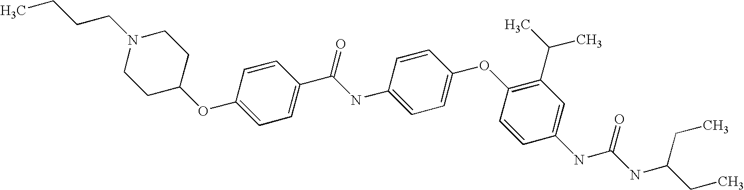 | 616 | 616 |
| 22 | 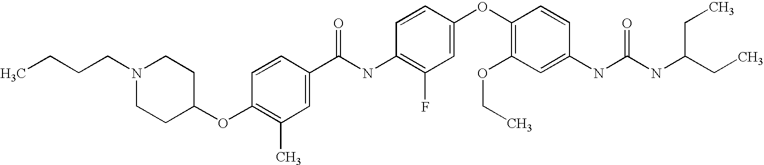 | 649 | 649 |
| 23 | 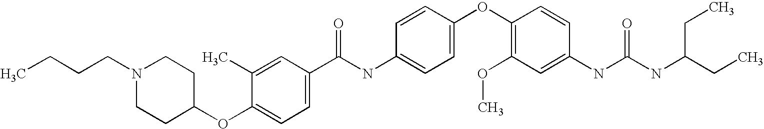 | 618 | 618 |
| 24 | 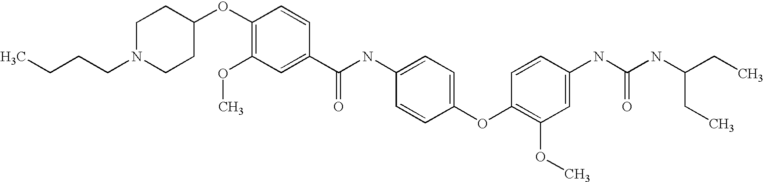 | 634 | 634 |
| 25 | 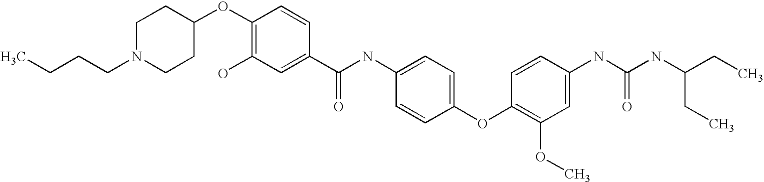 | 638 | 638 |
| 26 | 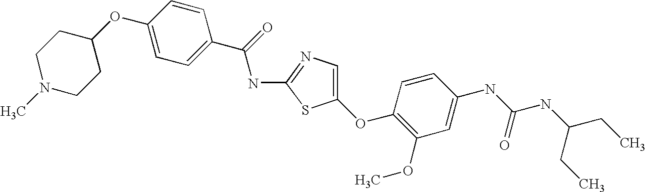 | 569 | 569 |
| 27 | 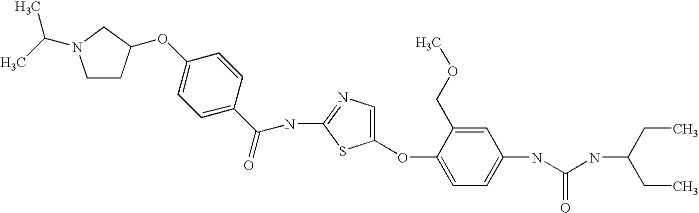 | 597 | 597 |
| 28 | 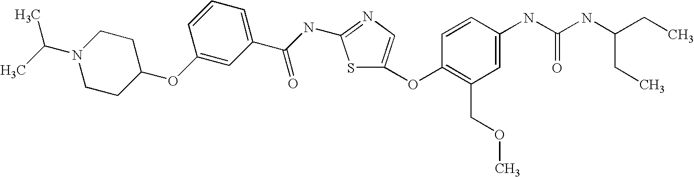 | 611 | 611 |
| 29 | 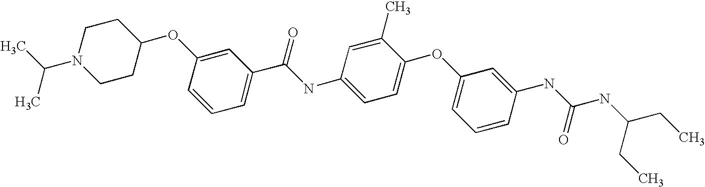 | 574 | 574 |
| 30 | 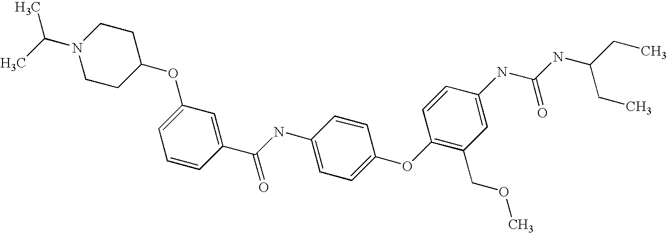 | 604 | 604 |
| 31 | 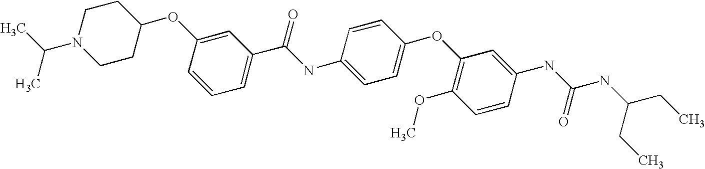 | 590 | 590 |
| 32 | 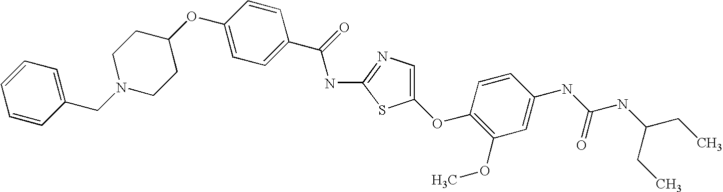 | 645 | 645 |
| 33 | 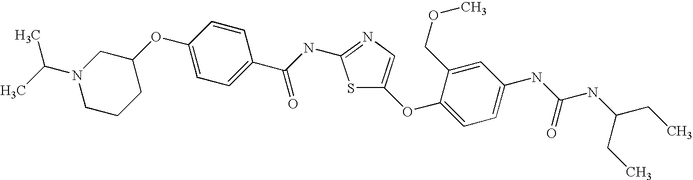 | 611 | 611 |
| 34 | 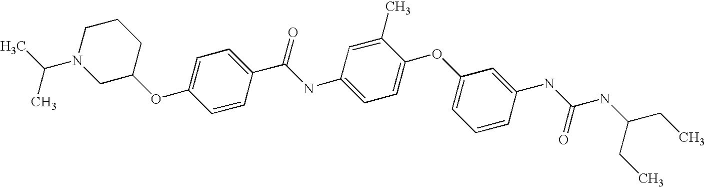 | 574 | 574 |
| 35 | 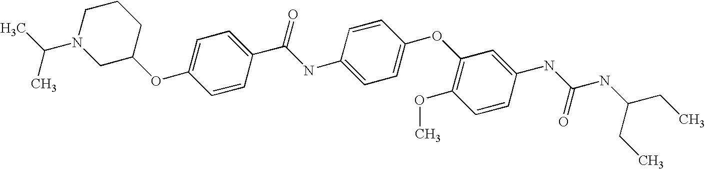 | 590 | 590 |
| 36 | 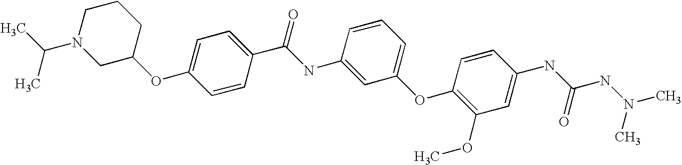 | 563 | 563 |
| 37 | 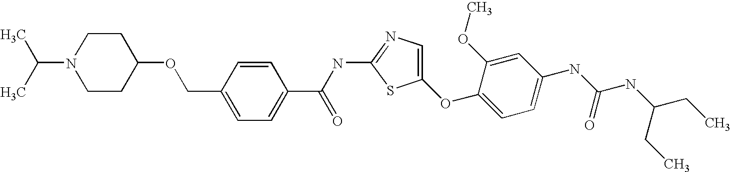 | 611 | 611 |
| 38 | 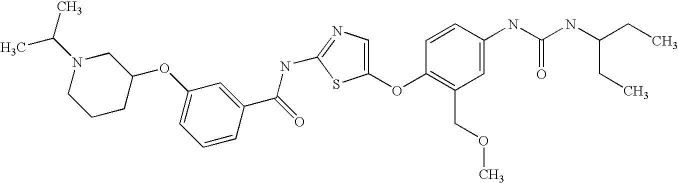 | 611 | 611 |
| 39 | 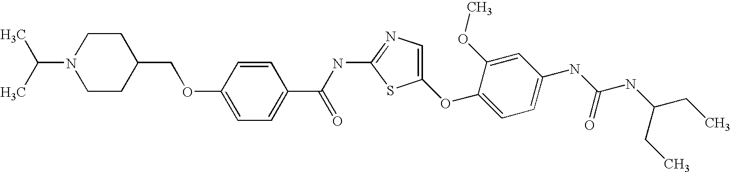 | 611 | 611 |
| 40 | 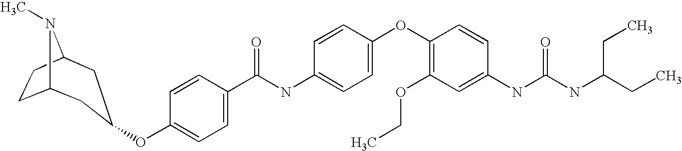 | 602 | 602 |
| 41 | 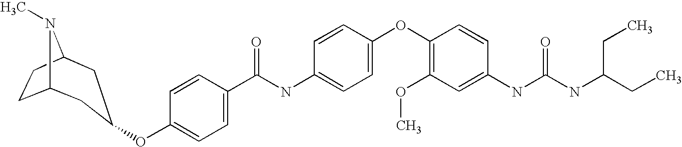 | 588 | 588 |
| 42 | 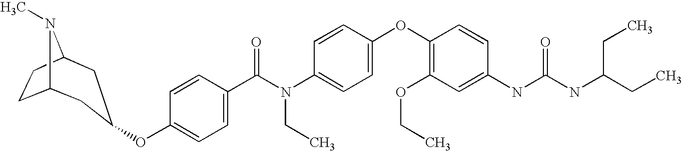 | 629 | 629 |
| 43 | 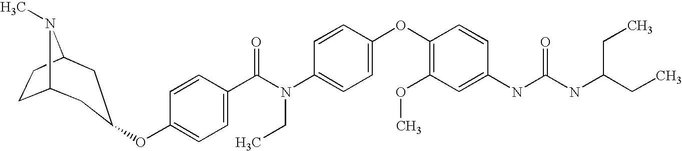 | 616 | 616 |
| 44 | 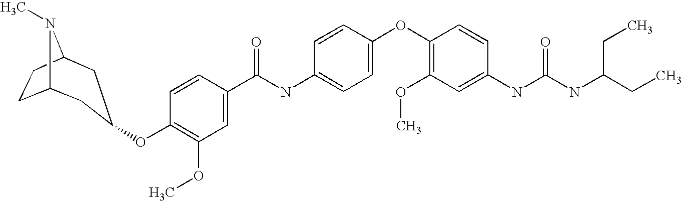 | 618 | 618 |
| 45 | 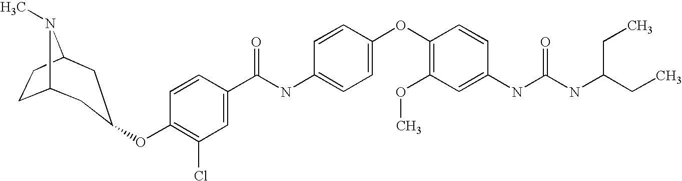 | 622 | 622 |
| 46 | 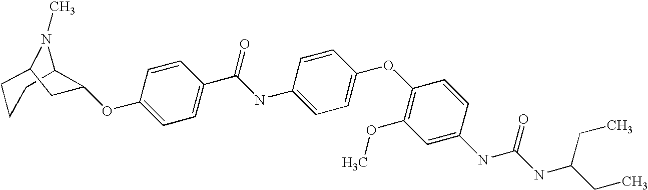 | 588 | 588 |
| 47 | 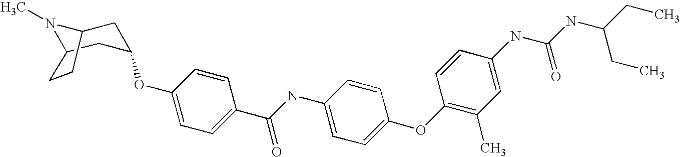 | 572 | 572 |
| 48 | 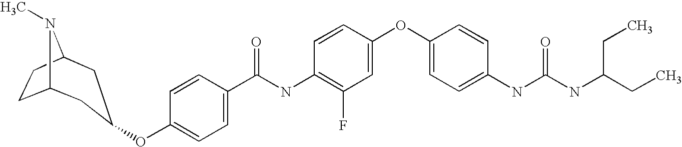 | 576 | 576 |
| 49 | 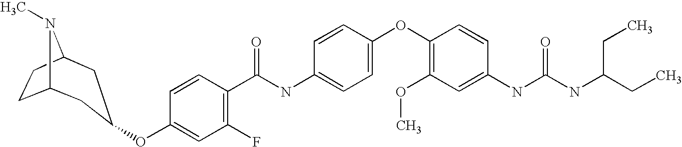 | 606 | 606 |
| 50 | 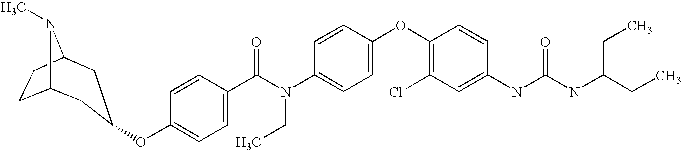 | 620 | 620 |
| 51 | 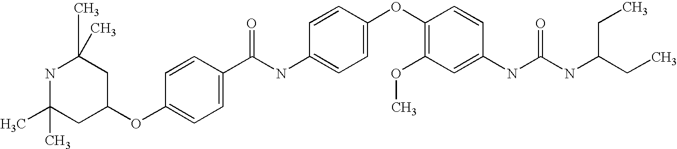 | 604 | 604 |
| 52 | 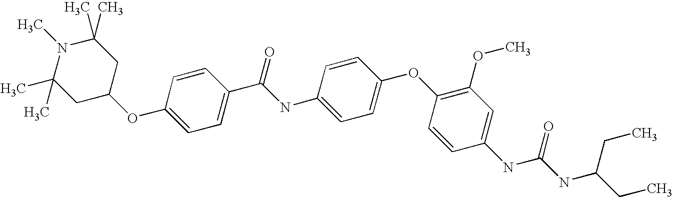 | 618 | 618 |
| 53 | 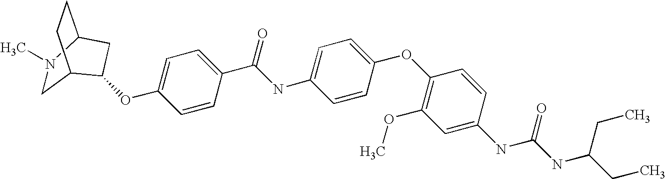 | 588 | 588 |
| 54 | 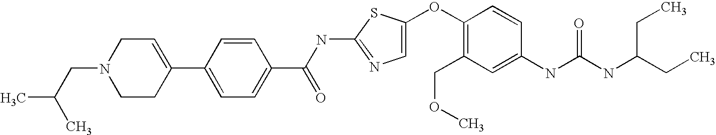 | 607 | 607 |
| 55 | 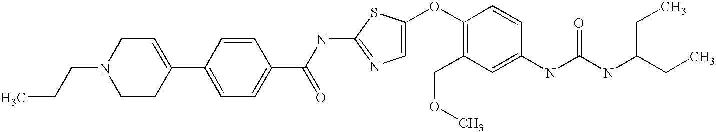 | 593 | 593 |
| 56 | 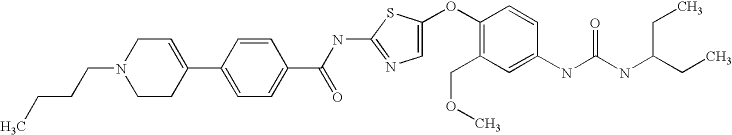 | 607 | 607 |
| 57 | 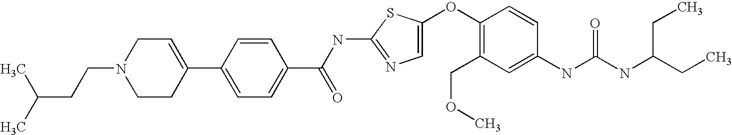 | 621 | 621 |
| 58 | 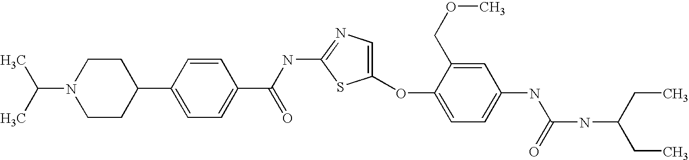 | 595 | 595 |
| 59 | 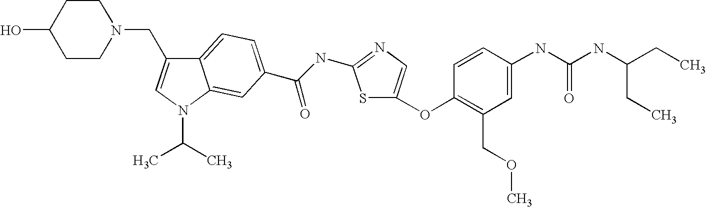 | 664 | 664 |
| 60 | 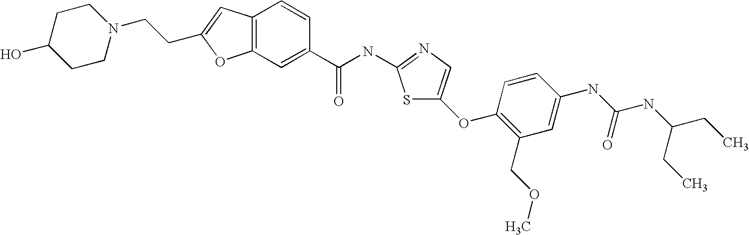 | 637 | 637 |
| 61 | 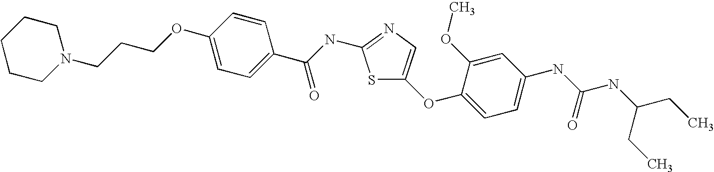 | 597 | 597 |
| 62 | 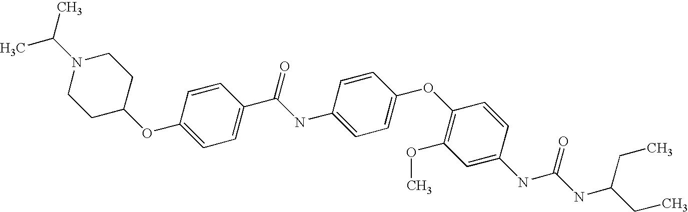 | 590 | 590 |
| 63 | 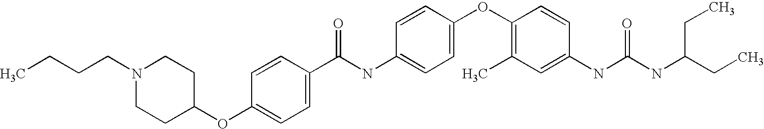 | 588 | 588 |
| 64 | 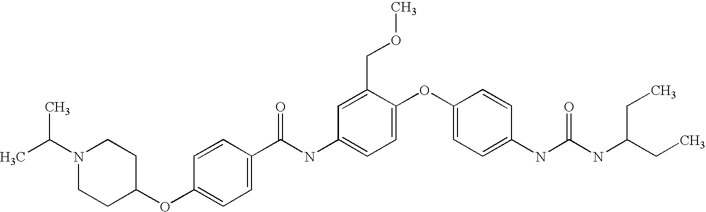 | 604 | 604 |
| 65 | 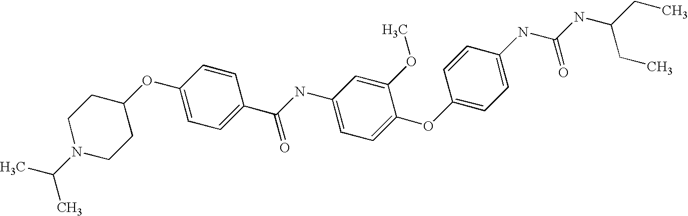 | 590 | 590 |
| 66 | 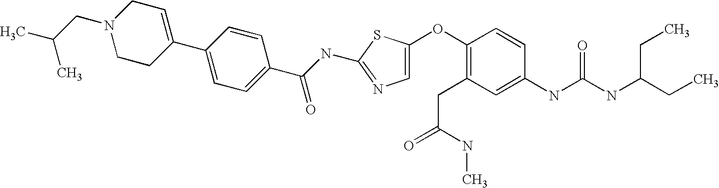 | 634 | 634 |
| 67 | 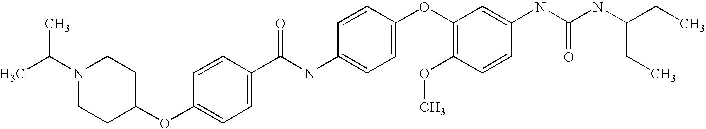 | 590 | 590 |
| 68 | 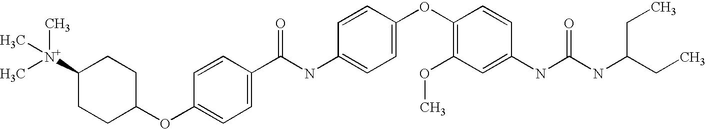 | 605 | 605 |
| 69 | 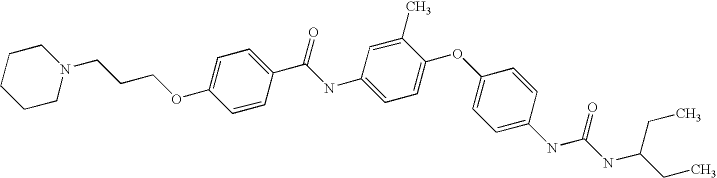 | 574 | 574 |
| 70 | 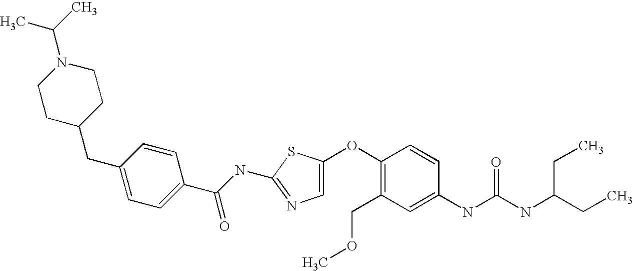 | 609 | 609 |
| 71 | 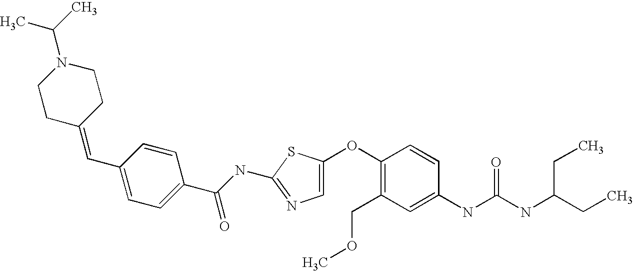 | 607 | 607 |
| 72 | 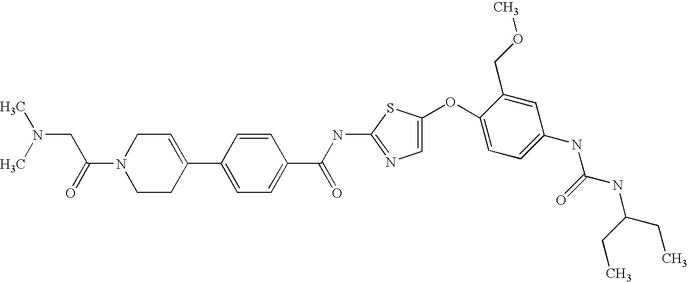 | 636 | 636 |
| 73 | 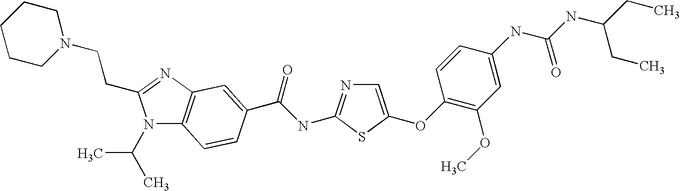 | 649 | 649 |
| 74 | 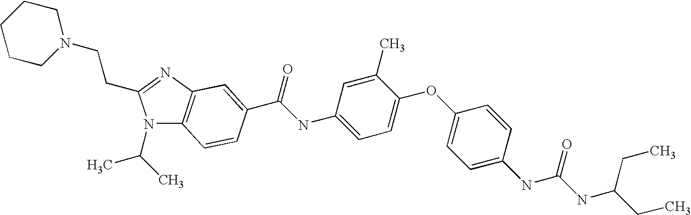 | 626 | 626 |
| 75 | 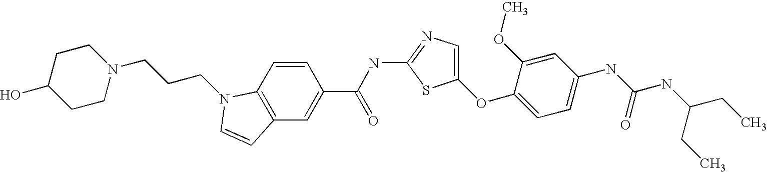 | 636 | 636 |
| 76 | 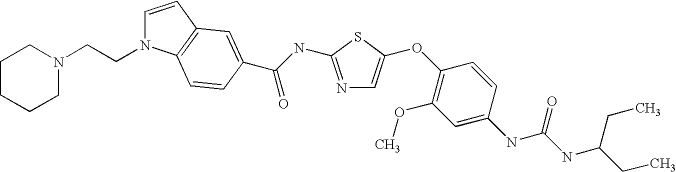 | 606 | 606 |
| 77 | 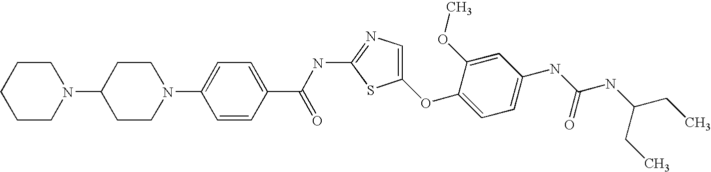 | 622 | 622 |
| 78 | 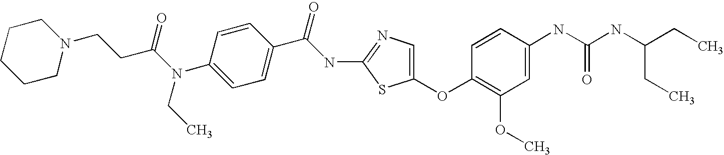 | 638 | 638 |
| 79 | 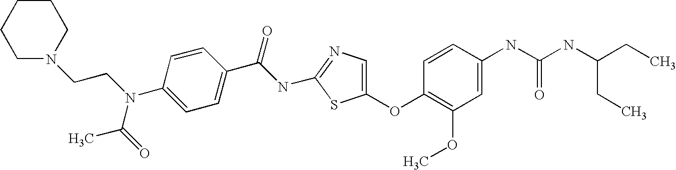 | 624 | 624 |
| 80 | 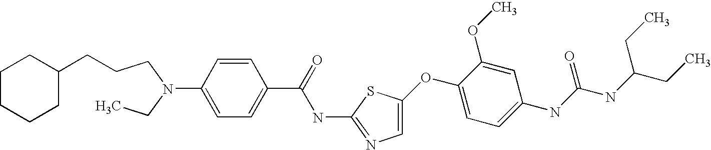 | 624 | 624 |
| 81 | 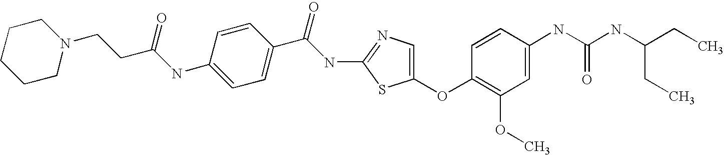 | 610 | 610 |
| 82 | 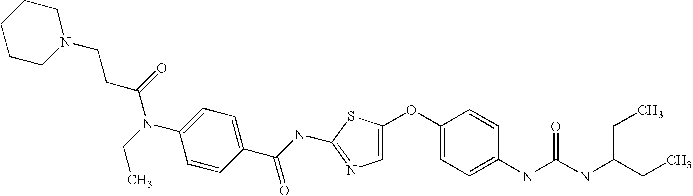 | 608 | 608 |
| 83 | 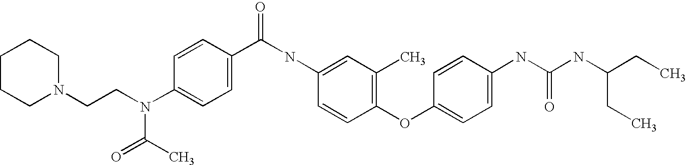 | 601 | 601 |
| 84 | 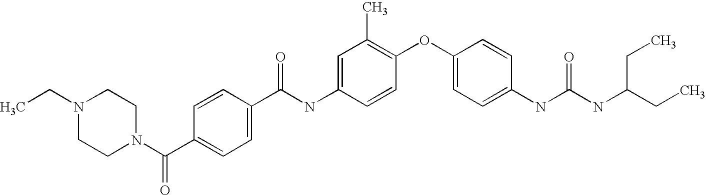 | 573 | 573 |
| 85 | 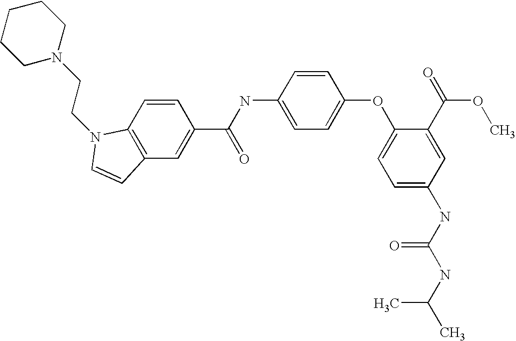 | 599 | 599 |
| 86 | 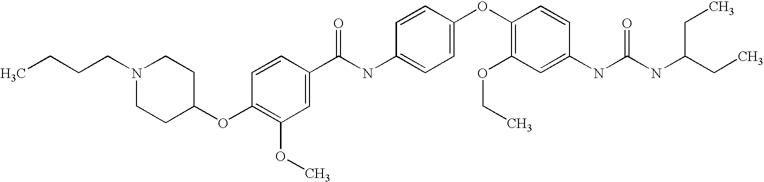 | 647 | 647 |
| 87 | 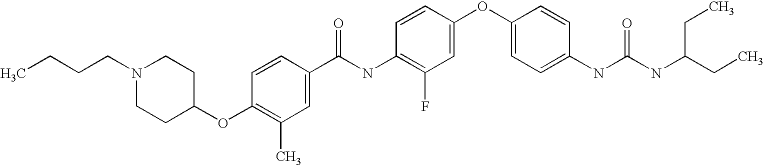 | 605 | 605 |
| 88 | 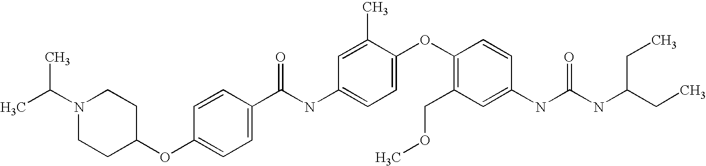 | 618 | 618 |
| 89 | 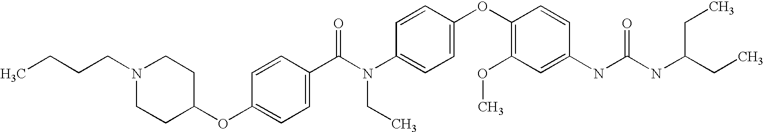 | 632 | 632 |
| 90 | 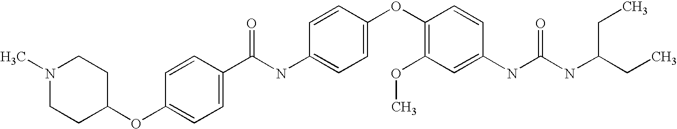 | 561 | 561 |
| 91 | 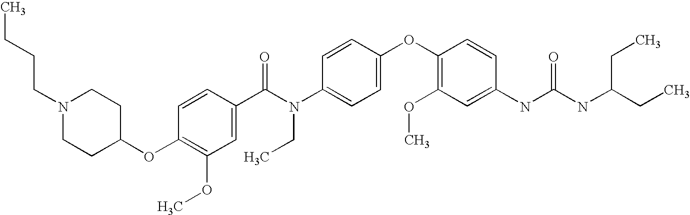 | 661 | 661 |
| 92 | 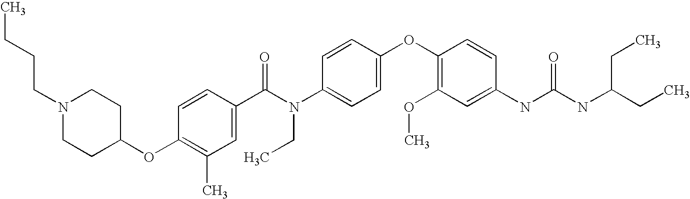 | 646 | 646 |
| 93 | 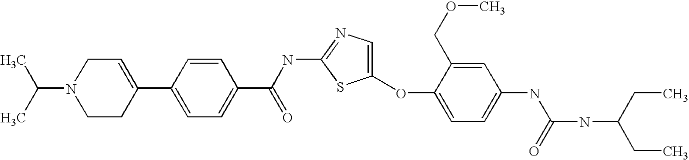 | 593 | 593 |
| 94 | 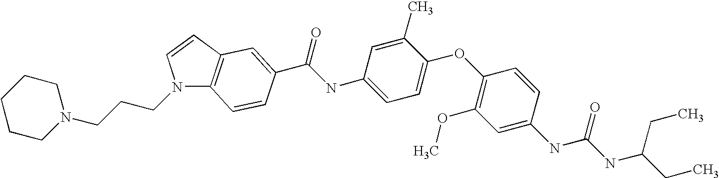 | 627 | 627 |
| 95 | 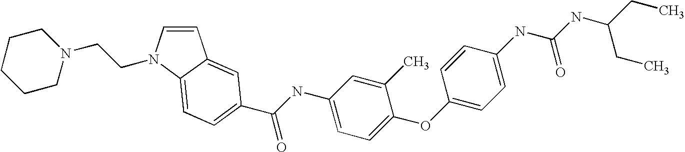 | 583 | 583 |
| 96 | 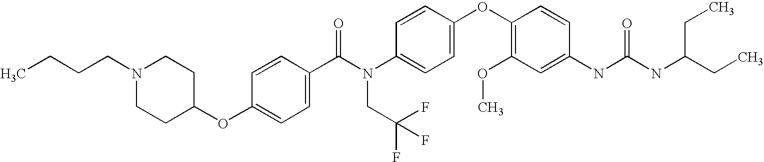 | 686 | 686 |
| 97 | 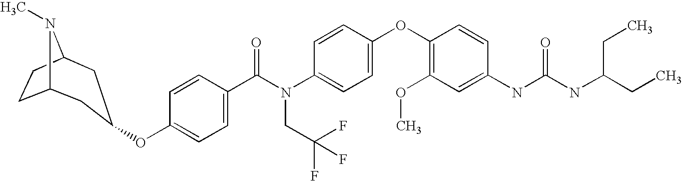 | 670 | 670 |
| 98 | 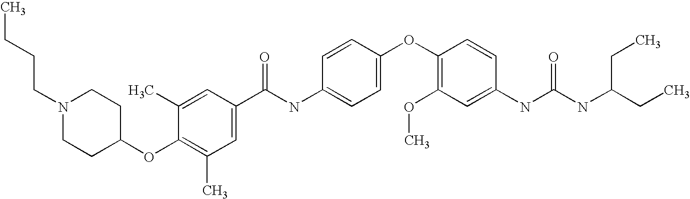 | 631 | 631 |
| 99 | 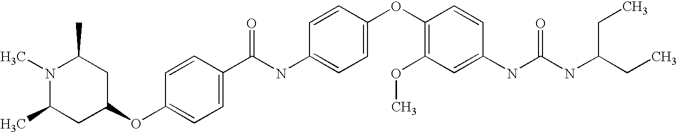 | 589 | 589 |
| 100 | 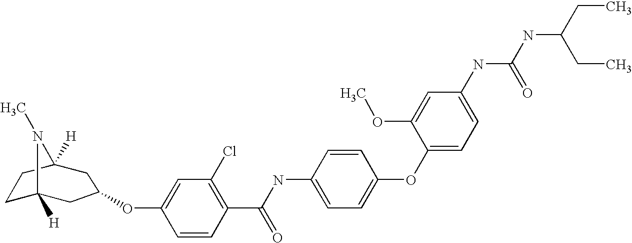 | 621 | 621 ^{(a)} |
| 101 | 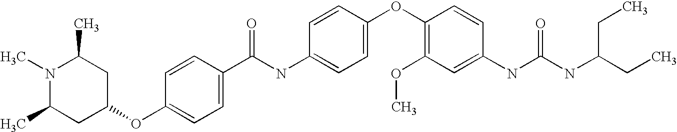 | 589 | 589 |
| 102 | 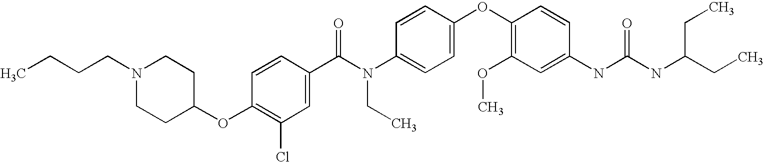 | 666 | 666 |
| 103 | 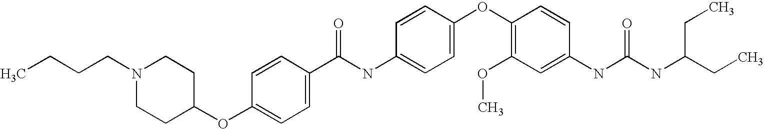 | 604 | 604 |
| 104 | 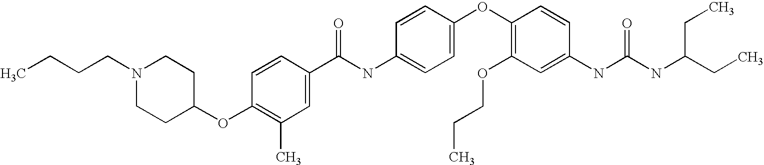 | 645 | 645 |
| 105 | 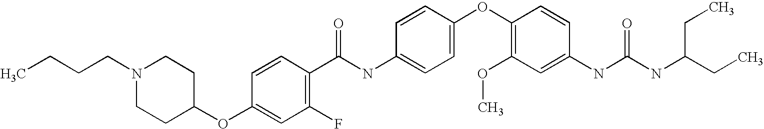 | 621 | 621 |
| 106 | 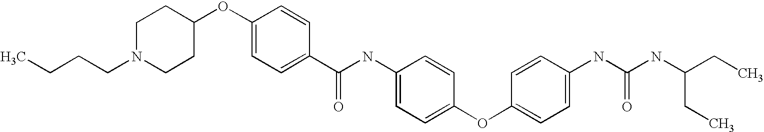 | 574 | 574 |
| 107 | 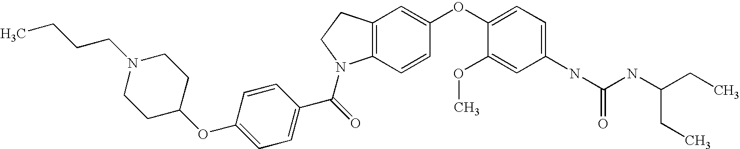 | 630 | 630 |
| 108 | 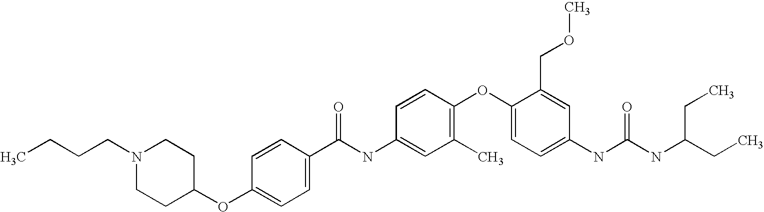 | 632 | 632 |
| 109 | 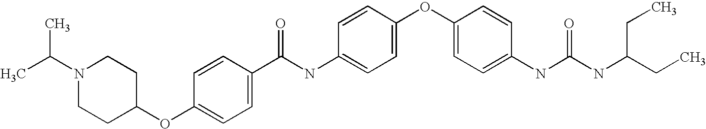 | 560 | 560 |
| 110 | 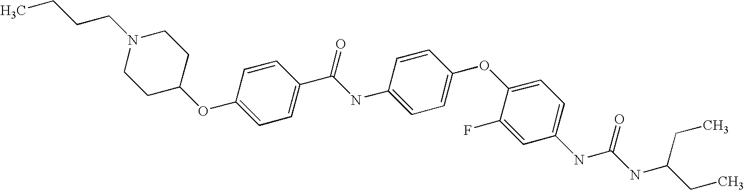 | 592 | 592 |
| 111 | 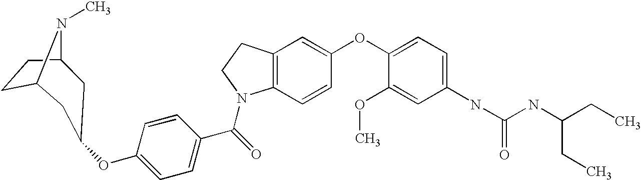 | 614 | 614 |
| 112 | 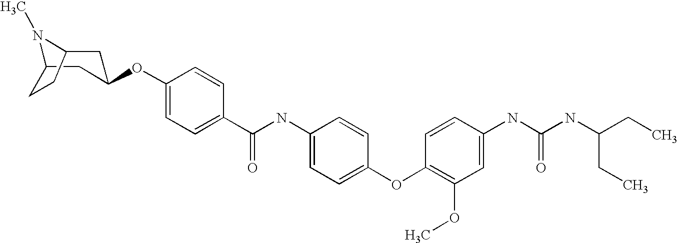 | 588 | 588 |
| 113 | 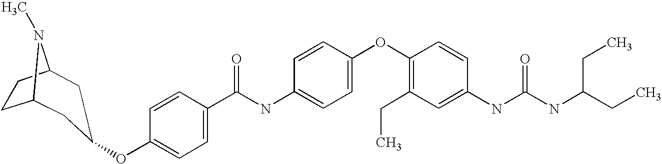 | 586 | 586 |
| 114 | 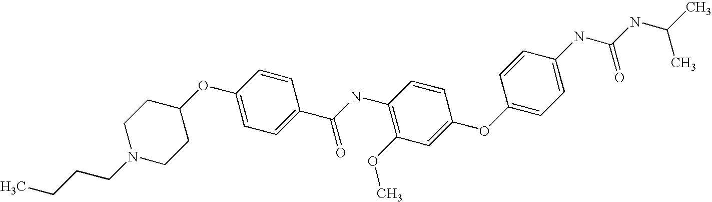 | 576 | 576 |
| 115 | 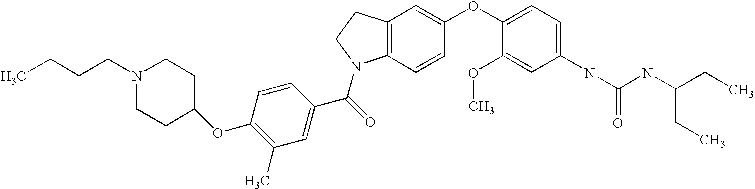 | 644 | 644 |
| 116 | 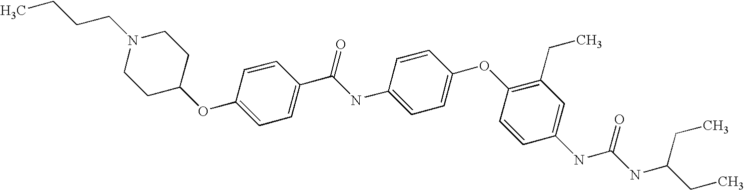 | 602 | 602 |
| 117 | 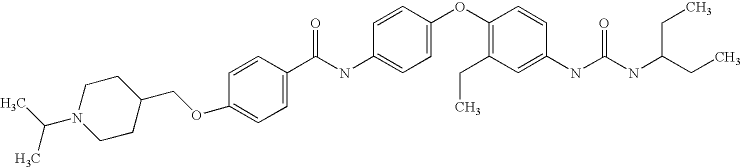 | 604 | 604 |
| 118 | 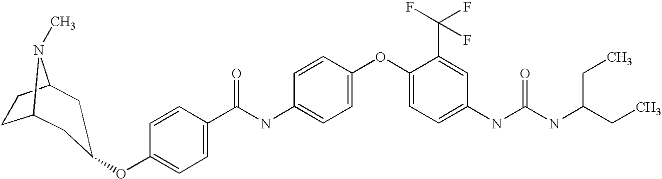 | 626 | 626 |
| 119 | 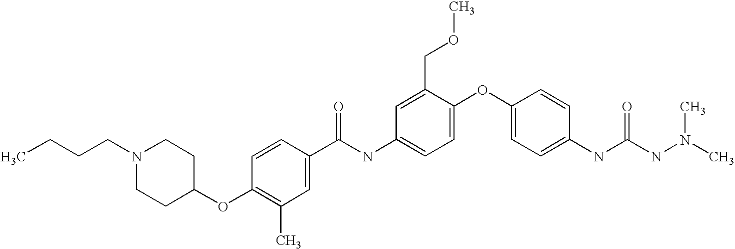 | 605 | 605 |
| 120 | 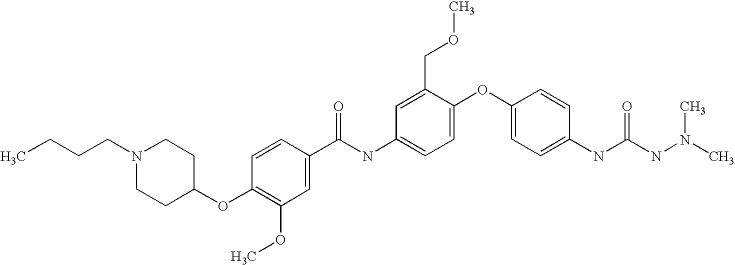 | 621 | 621 |
| 121 | 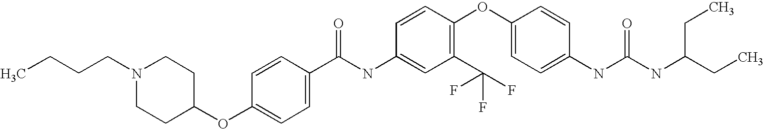 | 642 | 642 |
| 122 | 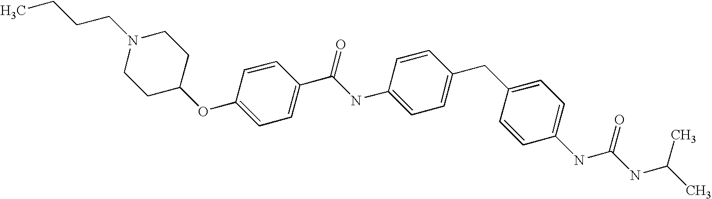 | 544 | 544 |
| 123 | 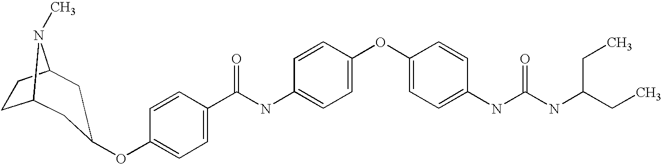 | 558 | 558 |
| 124 | 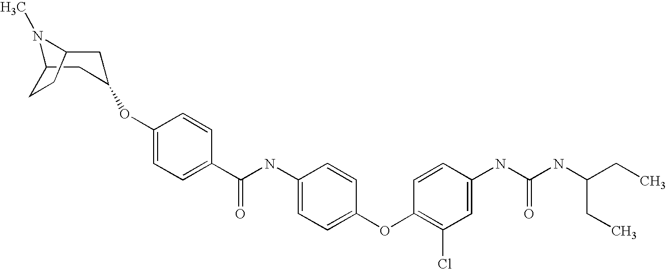 | 592 | 592 |
| 125 | 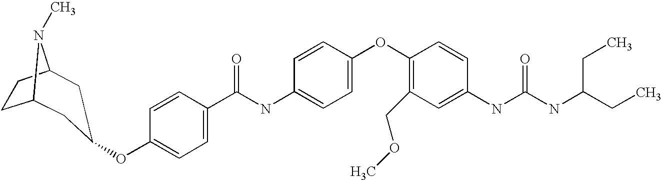 | 602 | 602 |
| 126 | 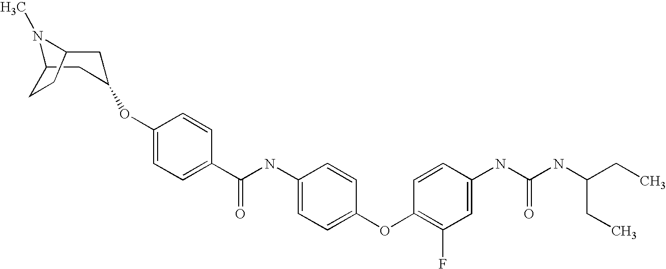 | 576 | 576 |
| 127 | 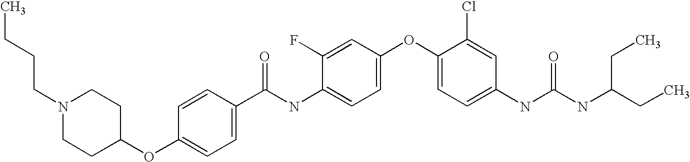 | 625 | 625 ^{(a)} |
| 128 | 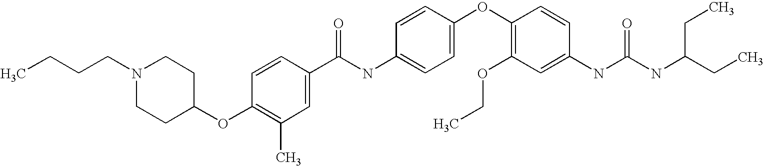 | 632 | 632 |
| 129 | 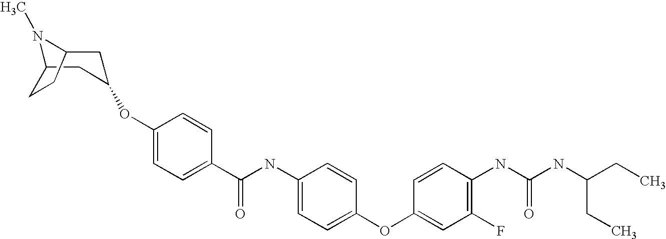 | 576 | 576 |
| 130 | 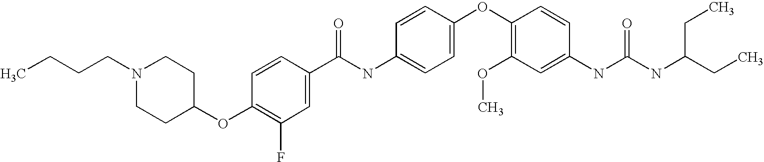 | 621 | 621 |
| 131 | 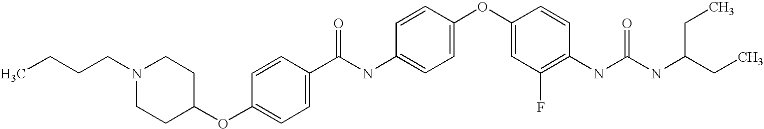 | 592 | 592 |
| 132 | 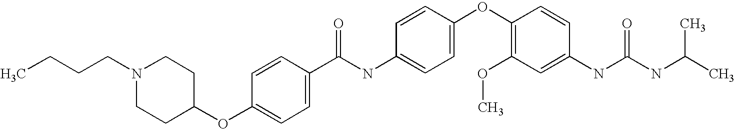 | 576 | 576 |
| 133 | 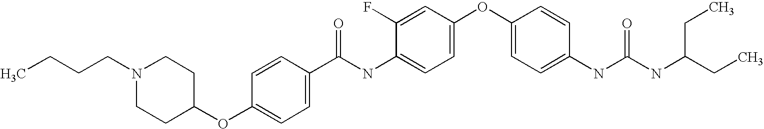 | 592 | 592 |
| 134 | 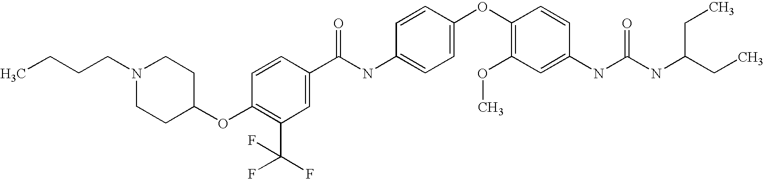 | 671 | 671 |
| 135 | 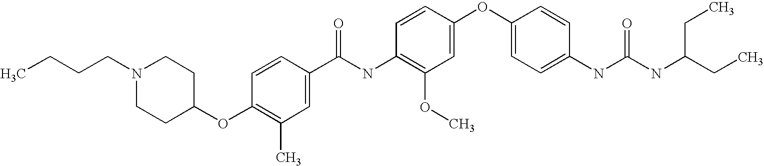 | 617 | 617 |
| 136 | 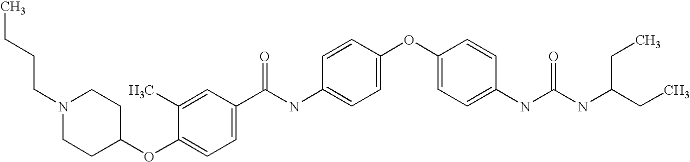 | 587 | 587 |
| 137 | 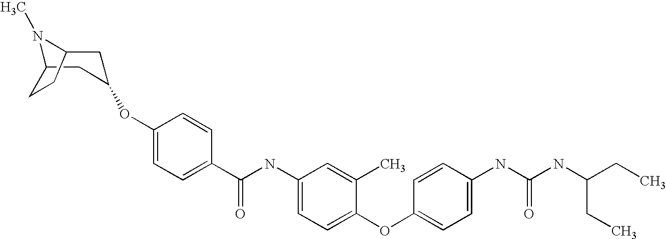 | 572 | 572 |
| 138 | 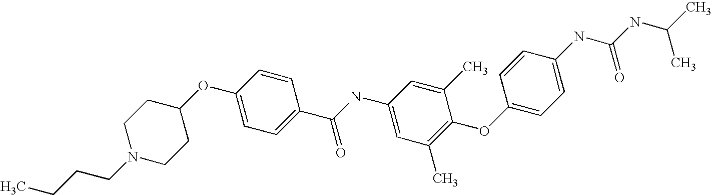 | 574 | 574 |
| 139 | 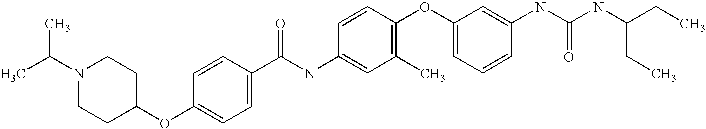 | 574 | 574 |
| 140 | 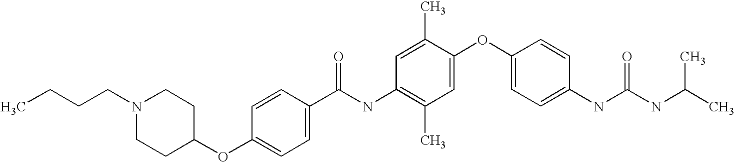 | 574 | 574 |
| 141 | 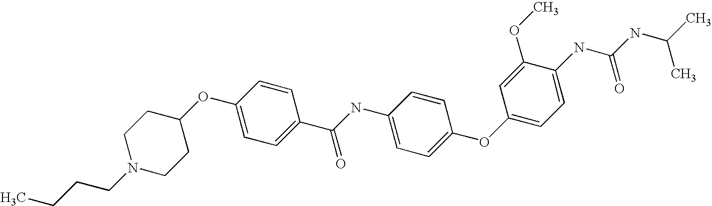 | 576 | 576 |
| 142 | 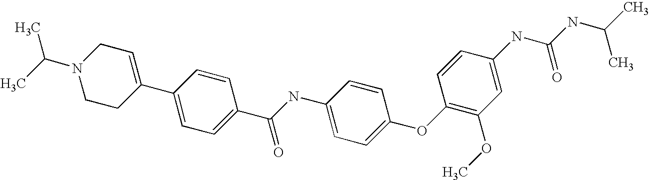 | 544 | 544 |
| 143 | 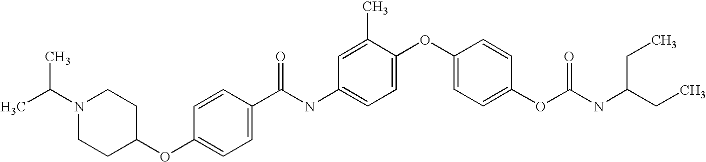 | 575 | 575 |
| 144 | 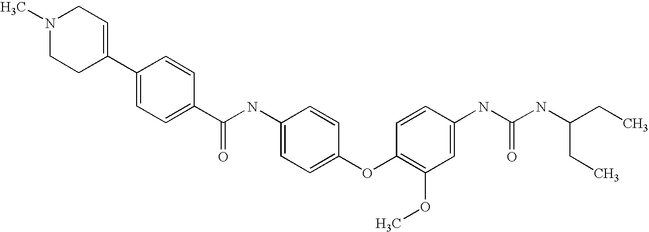 | 544 | 544 |
| 145 | 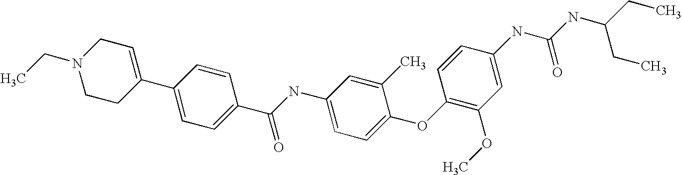 | 572 | 572 |
| 146 | 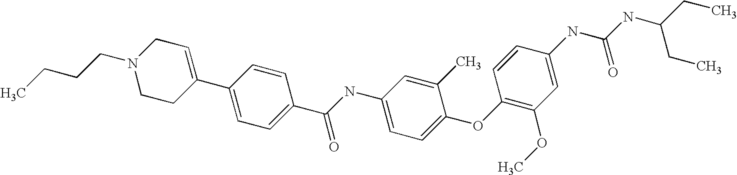 | 600 | 600 |
| 147 | 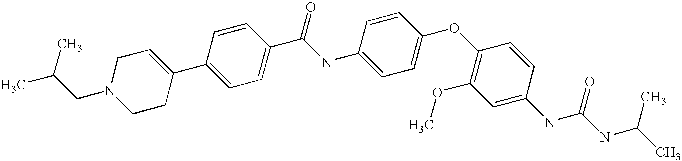 | 558 | 558 |
| 148 | 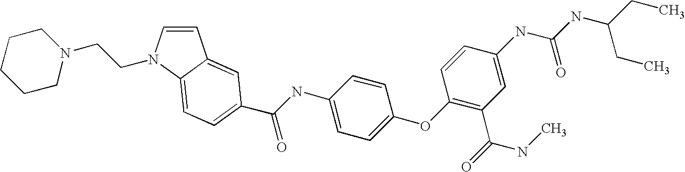 | 626 | 626 |
| 149 | 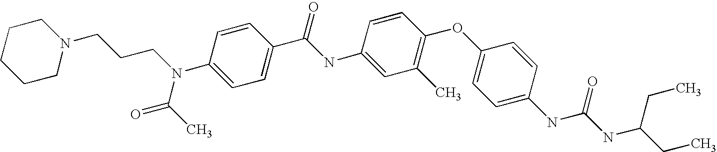 | 615 | 615 |
| 150 | 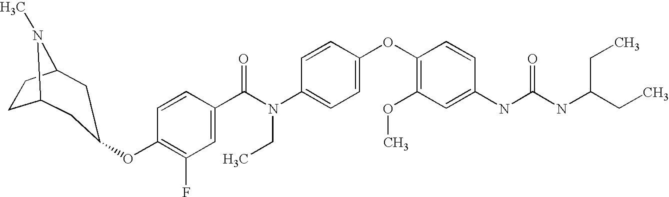 | 634 | 634 |
| 151 | 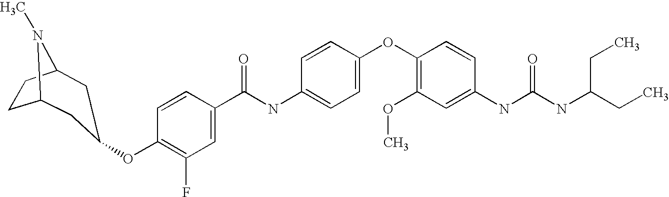 | 606 | 606 |
| 152 | 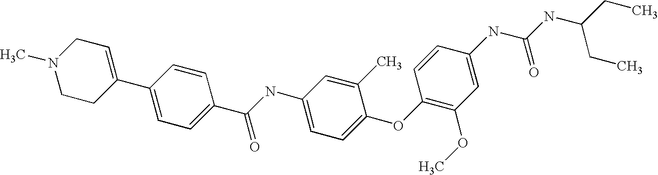 | 558 | 558 |
| 153 | 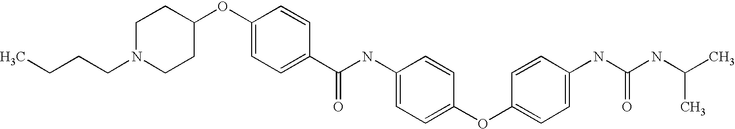 | 546 | 546 |
| 154 | 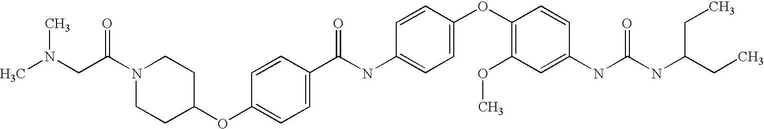 | 633 | 633 |
| 155 | 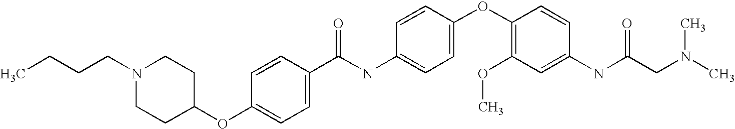 | 576 | 576 |
| 156 | 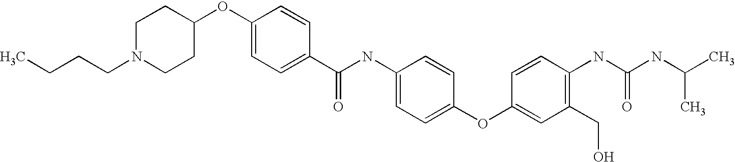 | 576 | 576 |
| 157 | 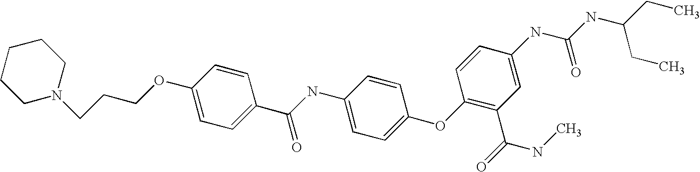 | 617 | 617 |
| 158 | 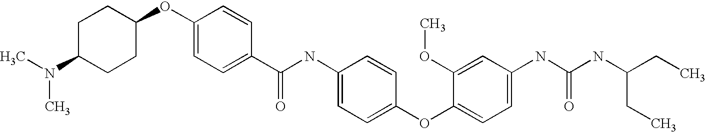 | 590 | 590 |
| 159 | 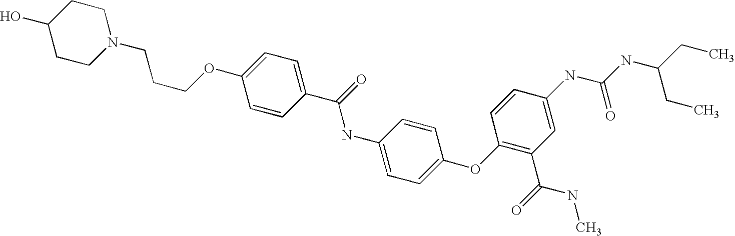 | 633 | 633 |
| 160 | 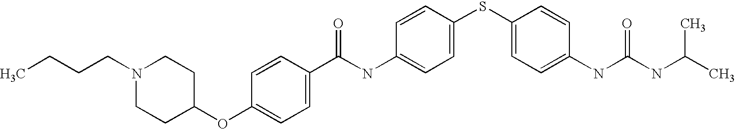 | 562 | 562 |
| 161 | 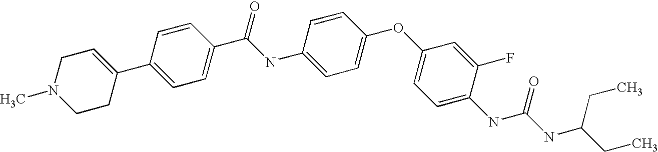 | 532 | 532 |
| 162 | 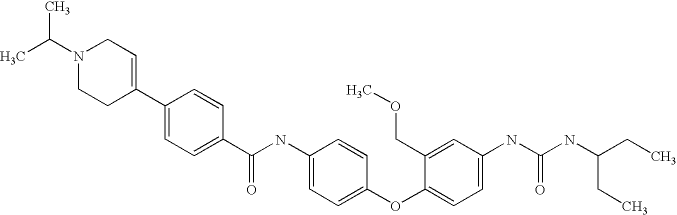 | 586 | 586 |
| 163 | 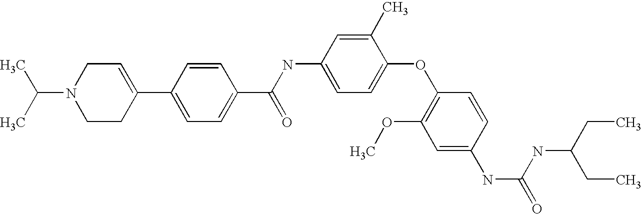 | 586 | 586 |
| 164 | 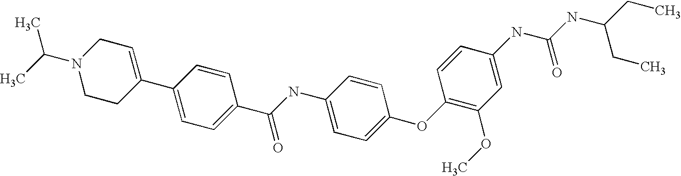 | 572 | 572 |
| 165 | 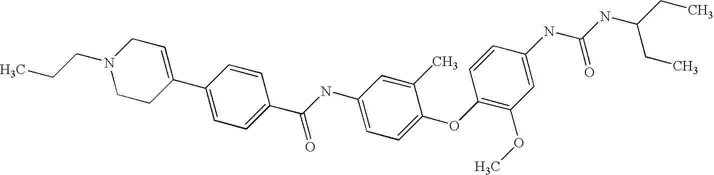 | 586 | 586 |
| 166 | 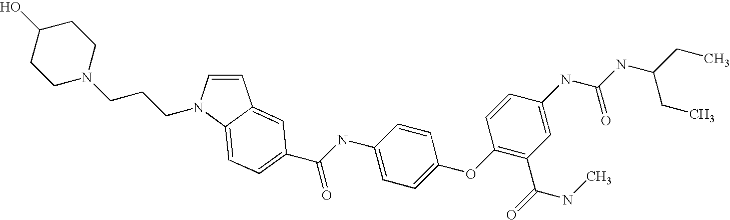 | 656 | 656 |
| 167 | 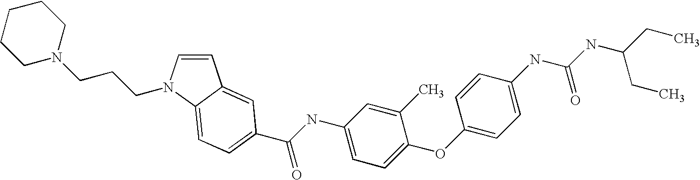 | 597 | 597 |
| 168 | 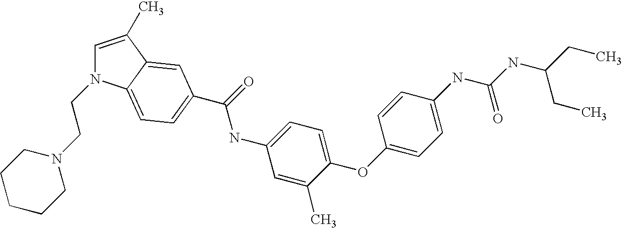 | 597 | 597 |
| 169 | 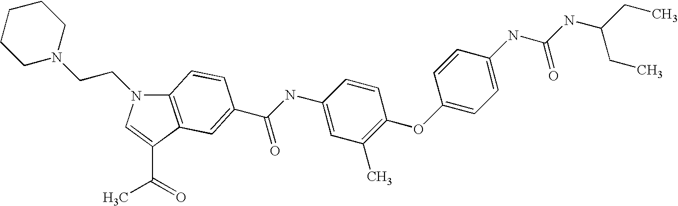 | 625 | 625 |
| 170 | 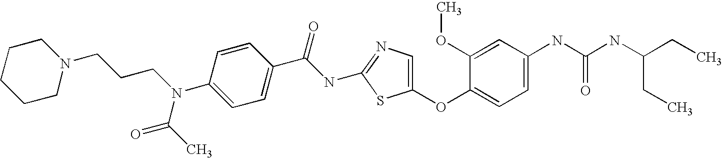 | 638 | 638 |
| 171 | 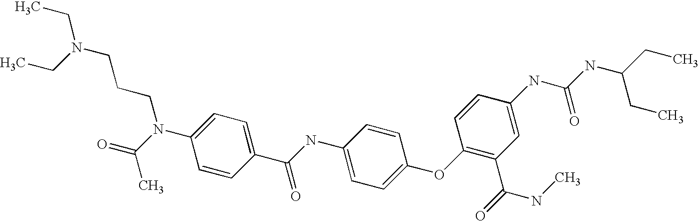 | 646 | 646 |
| 172 | 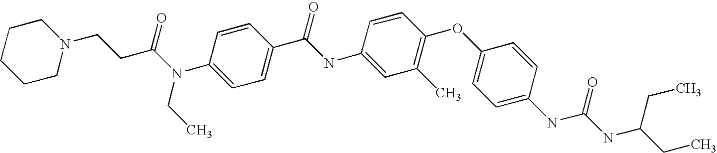 | 615 | 615 |
| 173 | 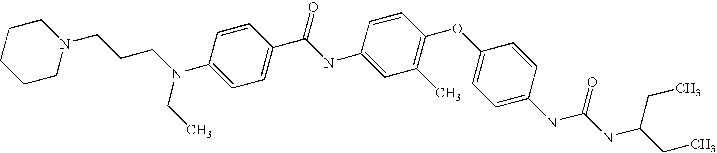 | 601 | 601 |
| 174 | 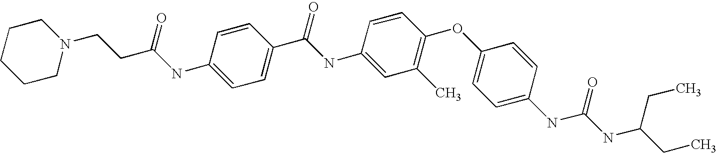 | 587 | 587 |
| 175 | 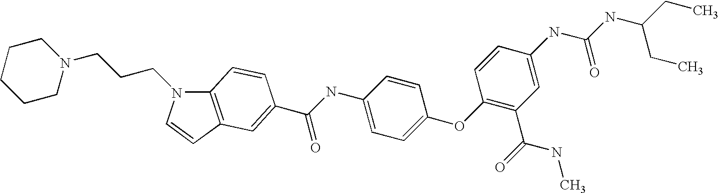 | 640 | 640 |
| 176 | 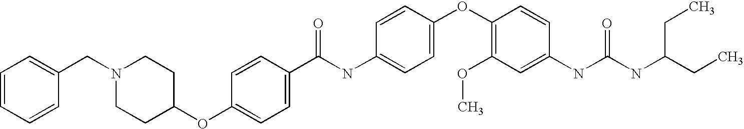 | 638 | 638 |
| 177 | 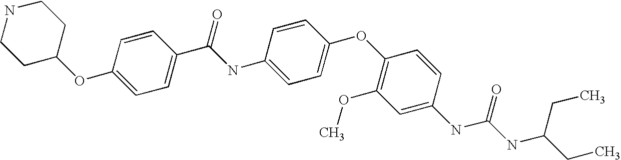 | 548 | 548 |
| 178 | 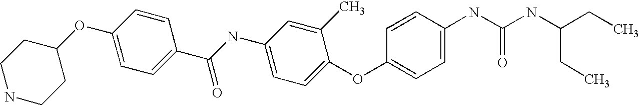 | 532 | 532 |
| 179 | 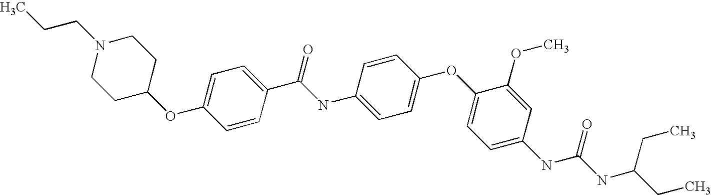 | 590 | 590 |
| 180 | 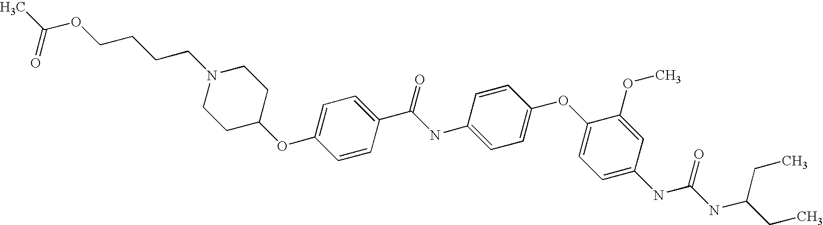 | 662 | 662 |
| 181 | 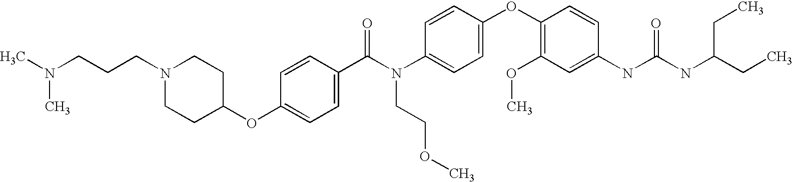 | 690 | 690 |
| 182 | 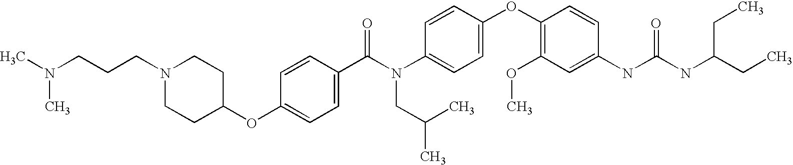 | 688 | 688 |
| 183 | 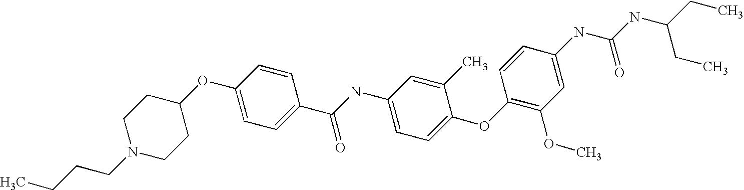 | 618 | 618 |
| 184 | 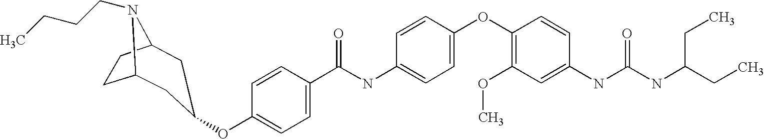 | 630 | 630 |
| 185 | 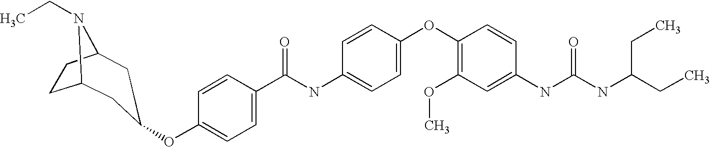 | 602 | 602 |
| 186 | 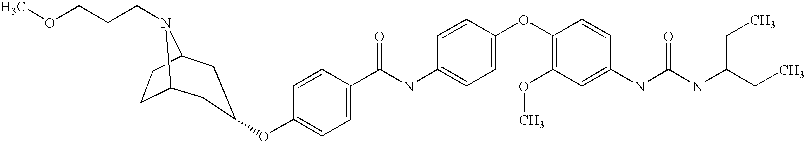 | 646 | 646 |
| 187 | 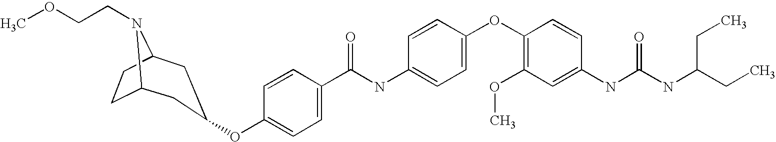 | 632 | 632 |
| 188 | 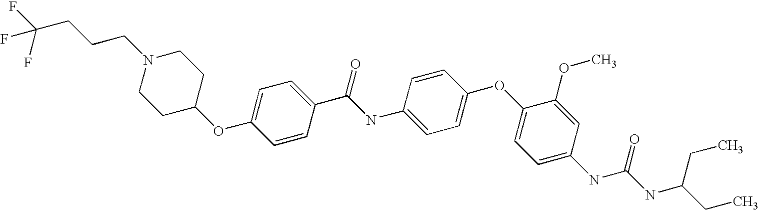 | 658 | 658 |
| 189 | 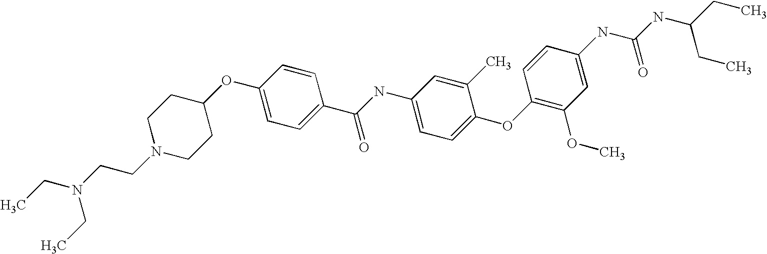 | 661 | 661 |
| 190 | 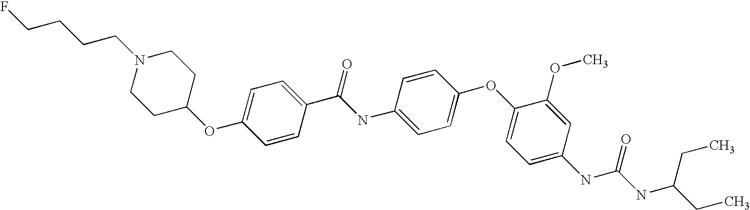 | 622 | 622 |
| 191 | 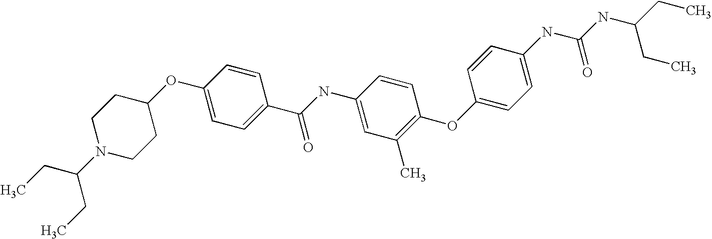 | 602 | 602 |
| 192 | 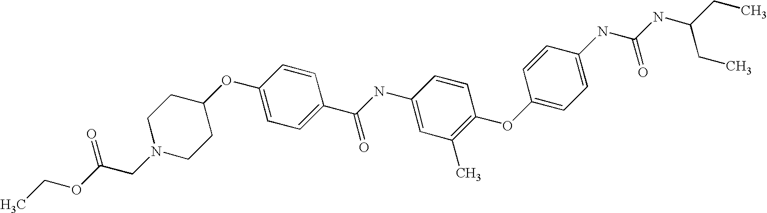 | 618 | 618 |
| 193 | 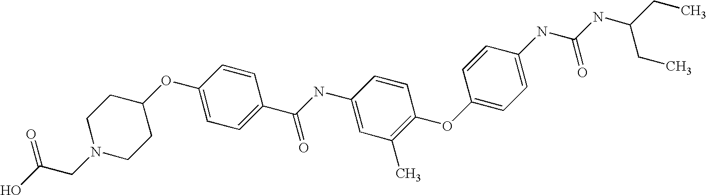 | 590 | 590 |
| 194 | 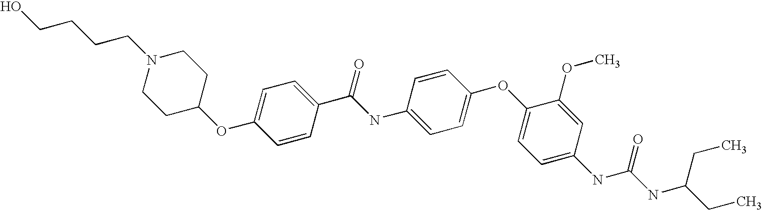 | 620 | 620 |
| 195 | 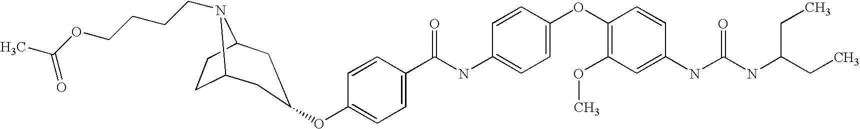 | 688 | 688 |
| 196 | 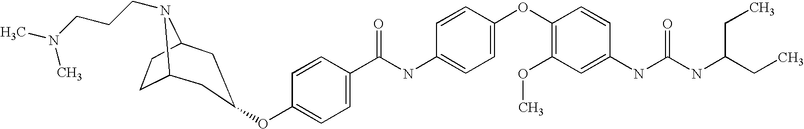 | 659 | 659 |
| 197 | 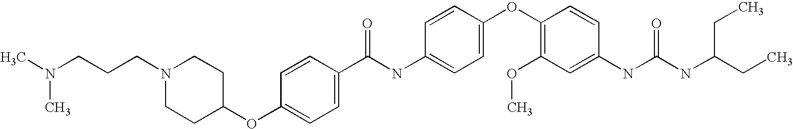 | 632 | 632 |
| 198 | 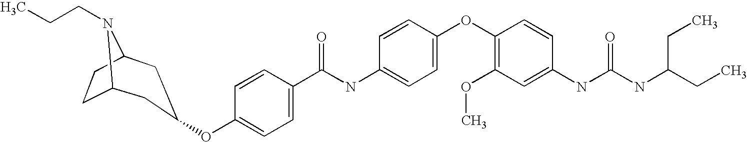 | 616 | 616 |
| 199 | 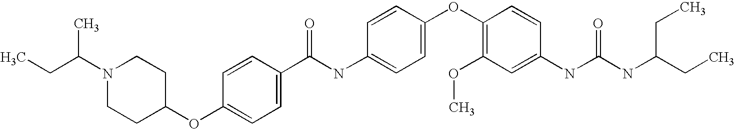 | 604 | 604 |
| 200 | 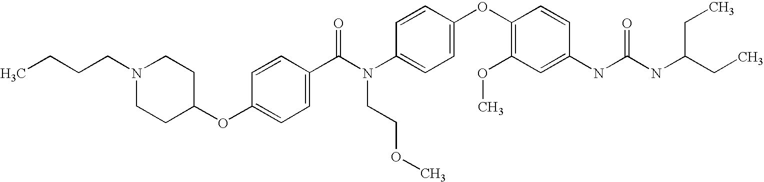 | 661 | 661 |
| 201 | 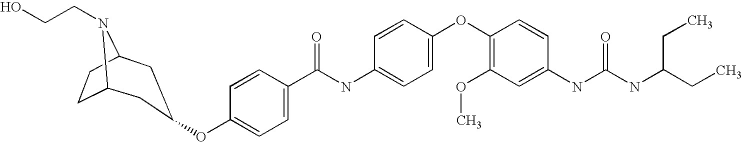 | 618 | 618 |
| 202 | 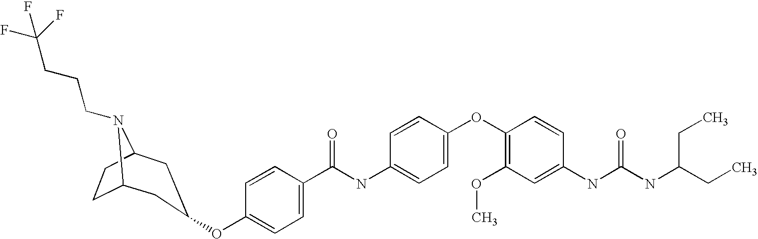 | 683 | 683 |
| 203 | 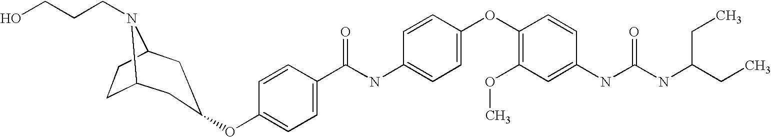 | 632 | 632 |
| 204 | 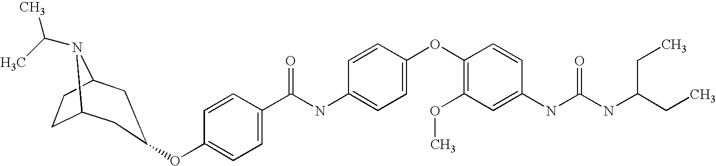 | 616 | 616 |
| 205 | 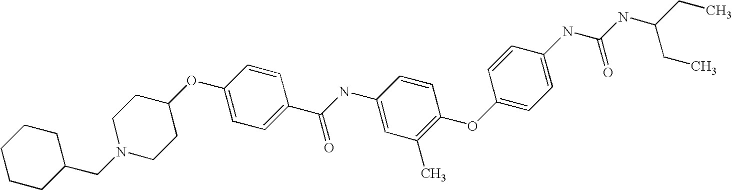 | 628 | 628 |
| 206 | 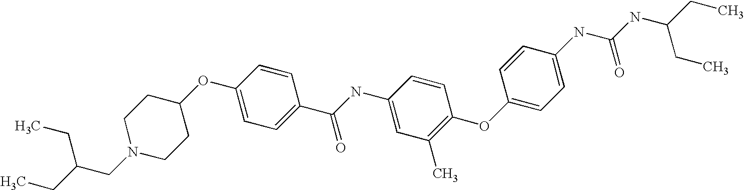 | 616 | 616 |
| 207 | 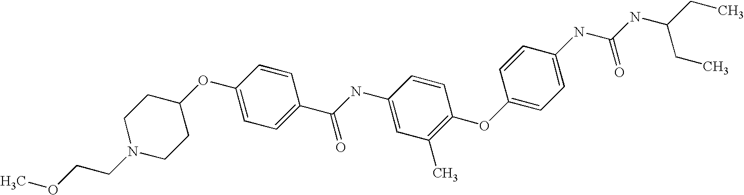 | 590 | 590 |
| 208 | 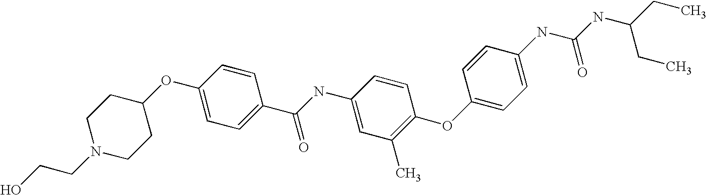 | 576 | 576 |
| 209 | 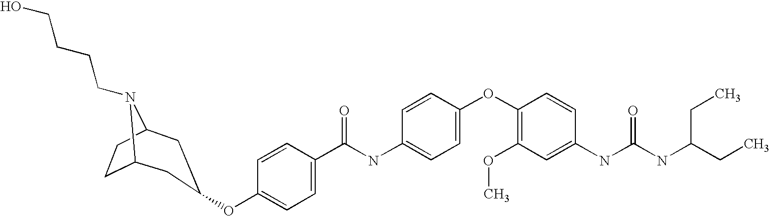 | 645 | 645 |
| 210 | 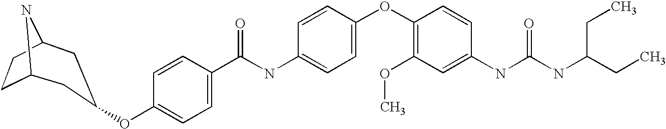 | 574 | 574 |
| 211 | 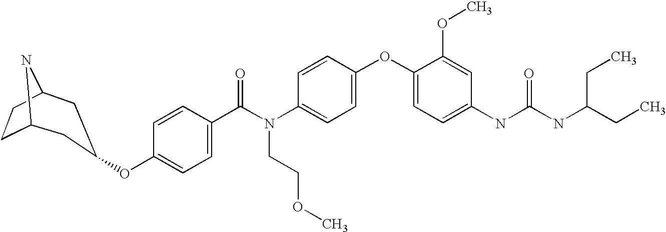 | 631 | 631 |
| 212 | 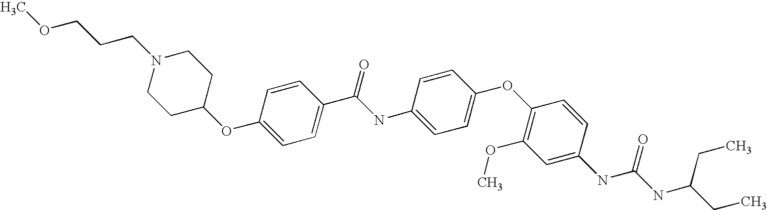 | 620 | 620 |
| 213 | 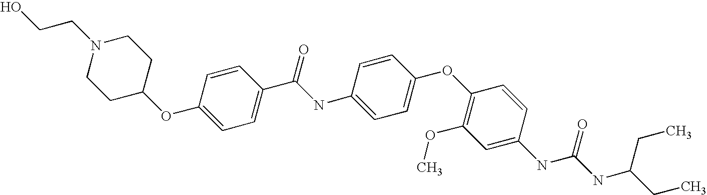 | 592 | 592 |
| 214 | 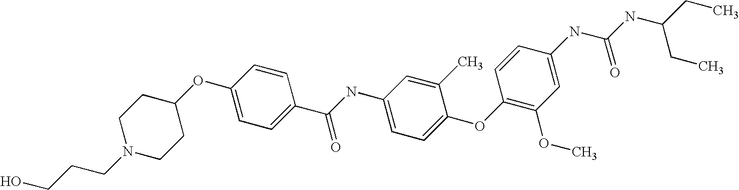 | 620 | 620 |
| 215 | 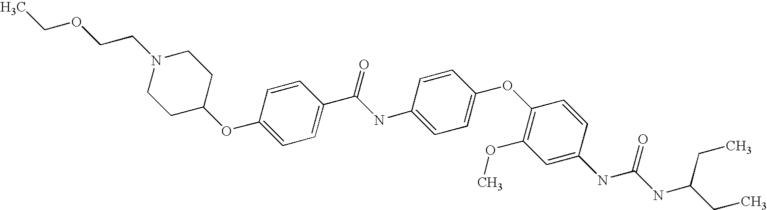 | 620 | 620 |
| 216 | 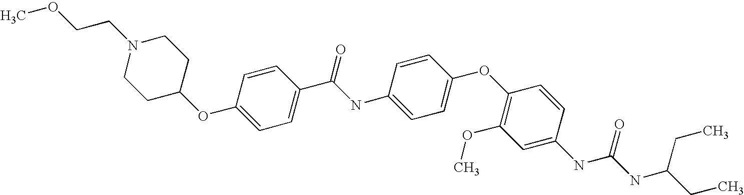 | 606 | 606 |
| 217 | 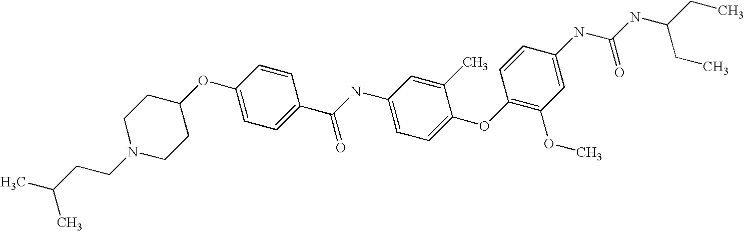 | 632 | 632 |
| 218 | 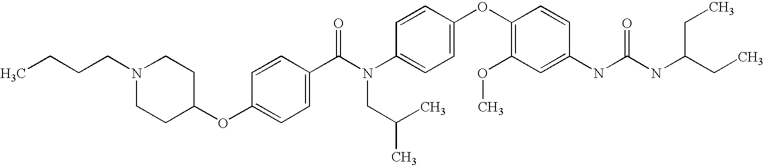 | 660 | 660 |
| 219 | 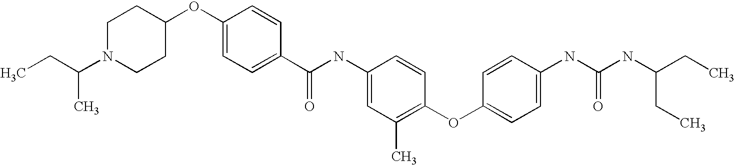 | 588 | 588 |
| 220 | 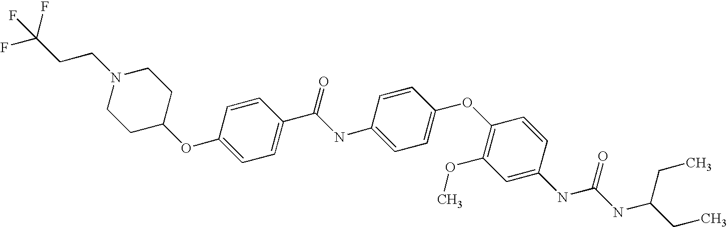 | 644 | 644 |
| 221 | 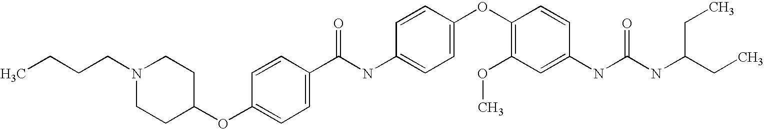 | 606 | 606 |
| 222 | 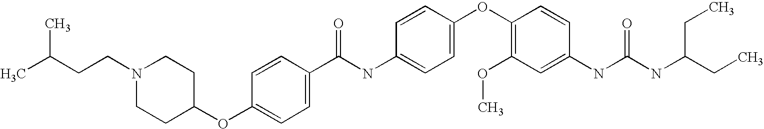 | 618 | 618 |
| 223 | 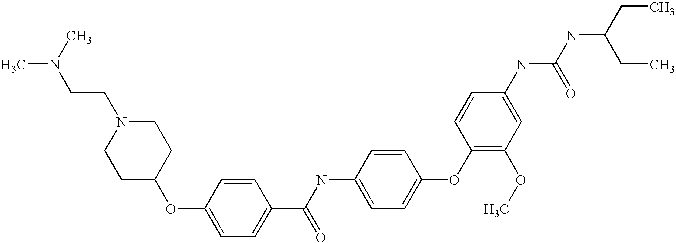 | 619 | 619 |
| 224 | 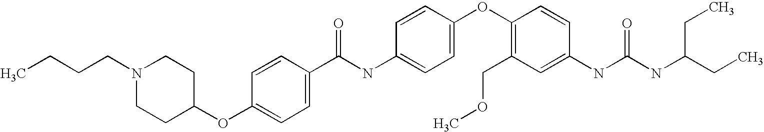 | 618 | 618 |
| 225 | 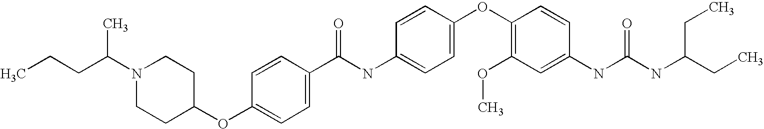 | 618 | 618 |
| 226 | 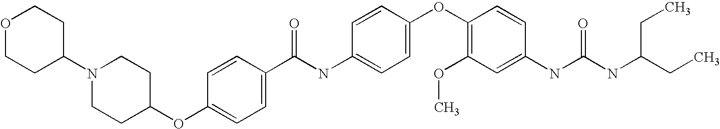 | 632 | 632 |
| 227 | 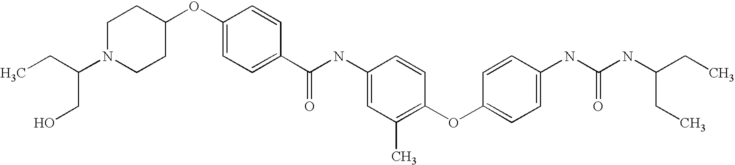 | 604 | 604 |
| 228 | 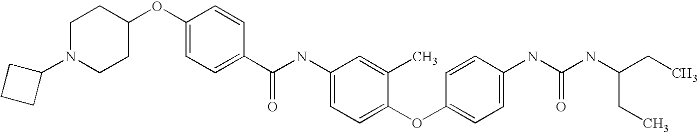 | 586 | 586 |
| 229 | 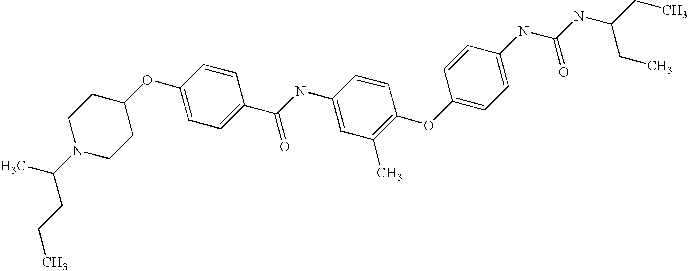 | 602 | 602 |
| 230 | 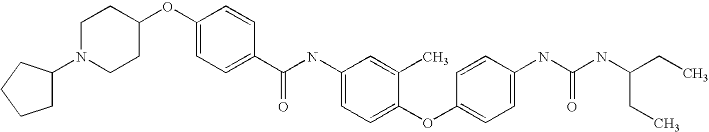 | 600 | 600 |
| 231 | 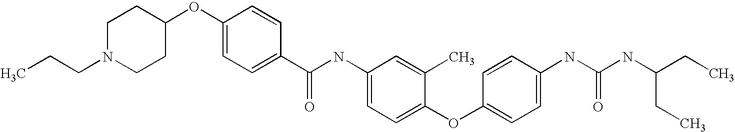 | 574 | 574 |
| 232 | 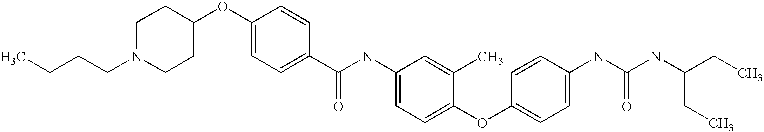 | 588 | 588 |
| 233 | 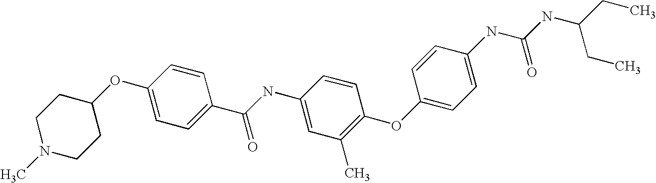 | 546 | 546 |
| 234 | 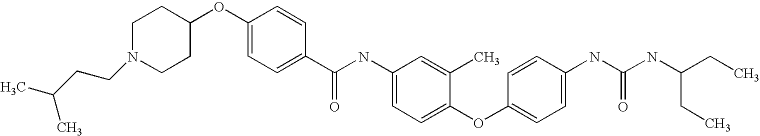 | 602 | 602 |
| 235 | 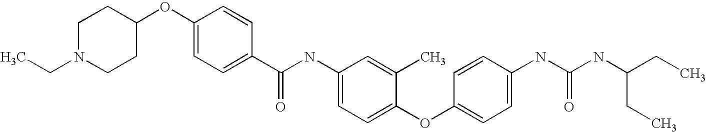 | 560 | 560 |
| 236 | 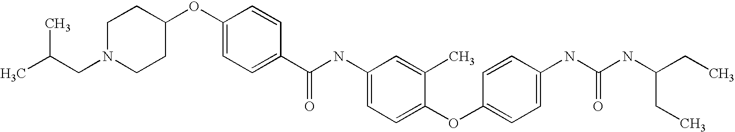 | 588 | 588 |
| 237 | 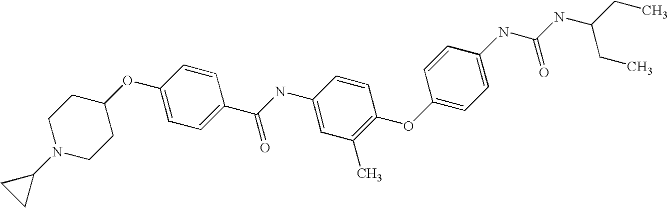 | 572 | 572 |
| 238 | 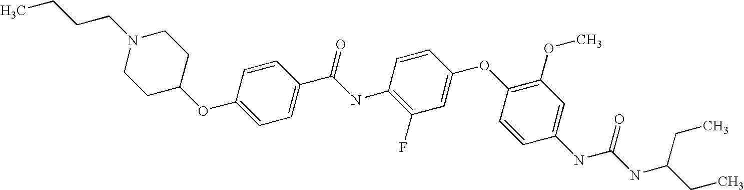 | 622 | 622 |
| 239 | 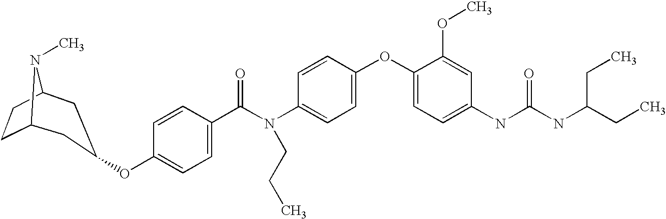 | 630 | 630 |
| 240 | 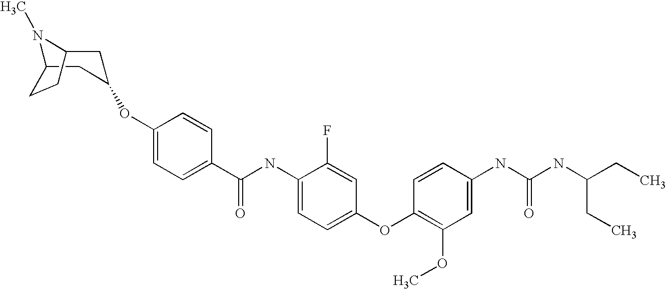 | 606 | 606 |
| 241 | 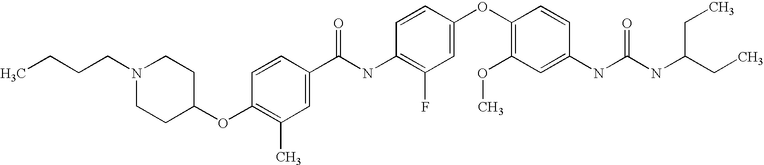 | 635 | 635 |
| 242 | 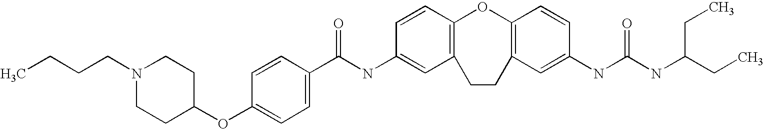 | 599 | 599 |
| 243 | 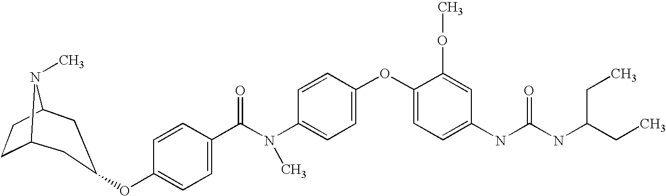 | 602 | 602 |
| 244 | 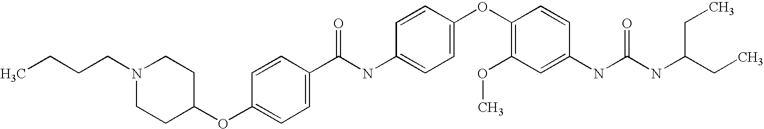 | 604 | 604 |
| 245 | 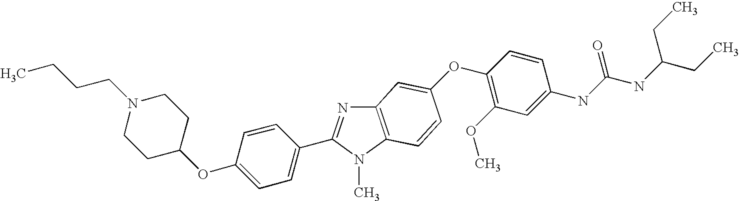 | 615 | 615 |
| 246 | 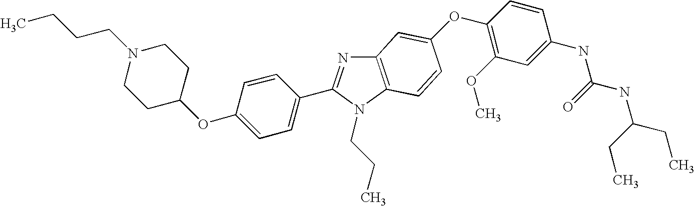 | 642 | 642 |
| 247 | 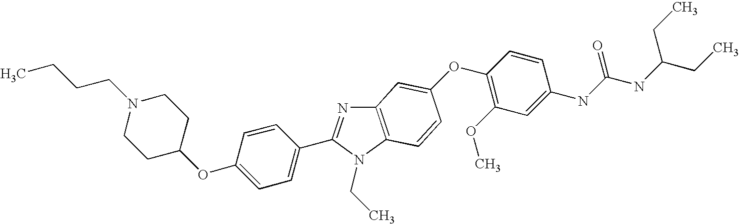 | 627 | 627 |
| 248 | 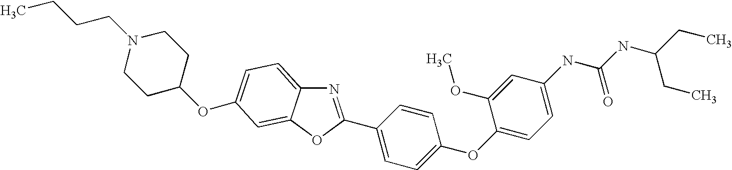 | 601 | 601 |
| 249 | 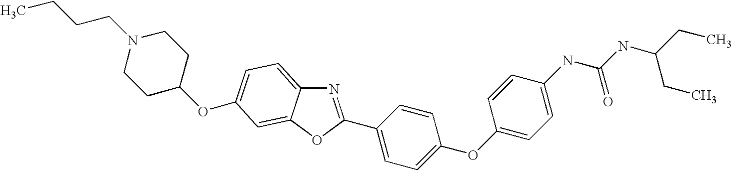 | 571 | 571 |
| 250 | 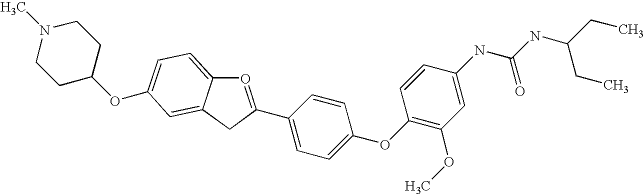 | 558 | 558 |
| 251 | 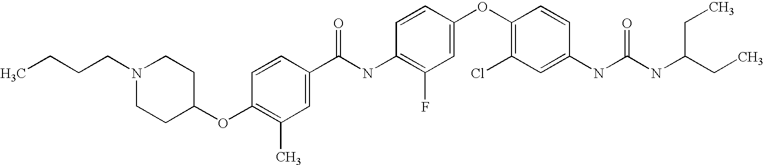 | 640 | 640 |
| 252 | 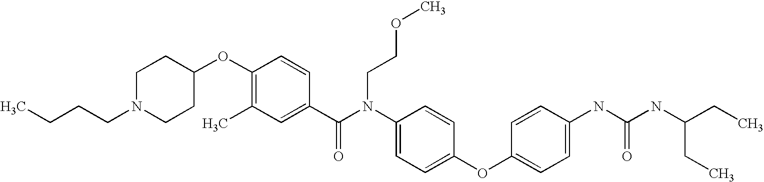 | 645 | 645 |
| 253 | 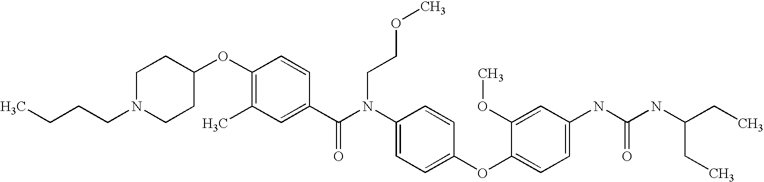 | 675 | 675 |
| 254 | 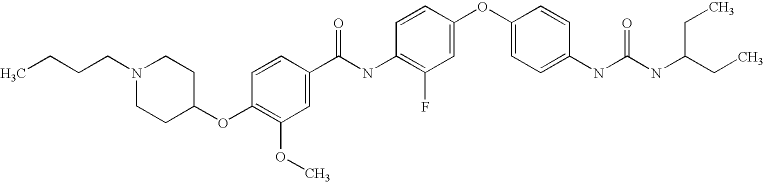 | 621 | 621 |
| 255 | 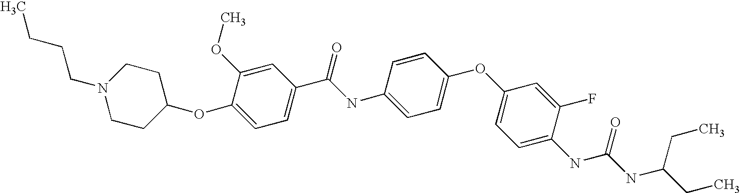 | 621 | 621 |
| 256 | 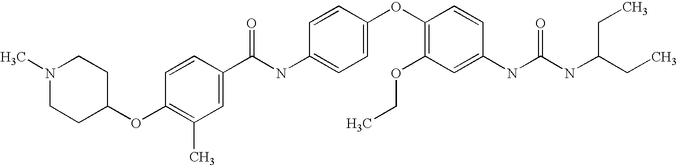 | 589 | 589 |
| 257 | 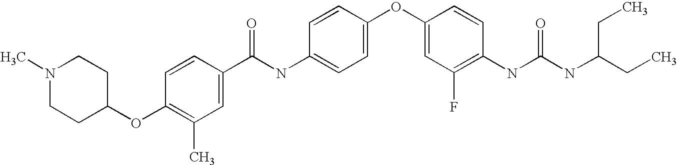 | 563 | 563 |
| 258 | 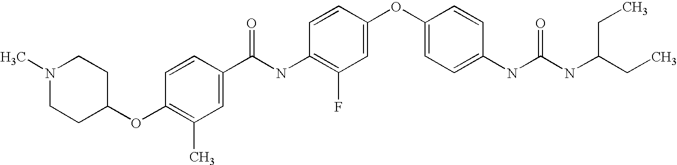 | 563 | 563 |
| 259 | 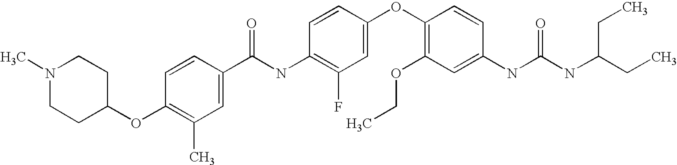 | 607 | 607 |
| 260 | 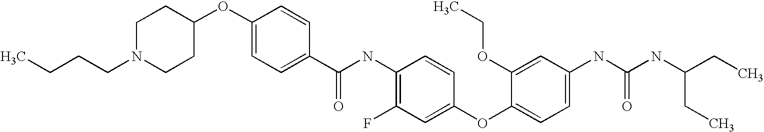 | 635 | 635 |
| 261 | 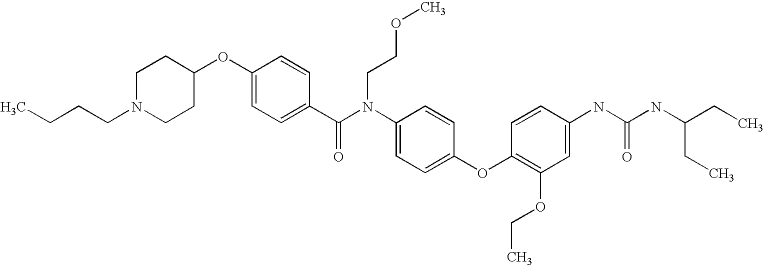 | 675 | 675 |
| 262 | 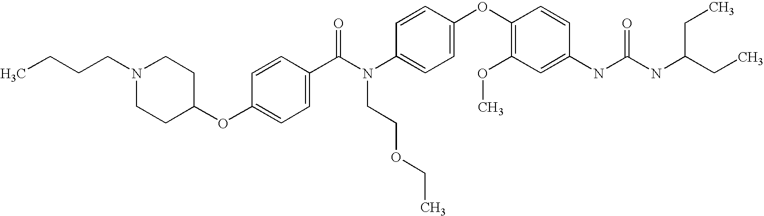 | 675 | 675 |
| 263 | 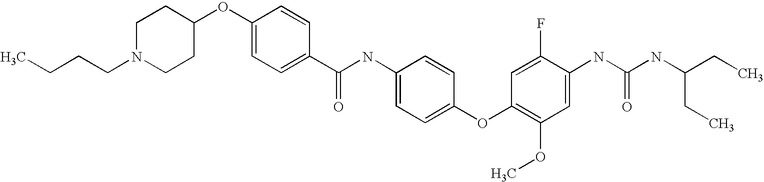 | 621 | 621 |
| 264 | 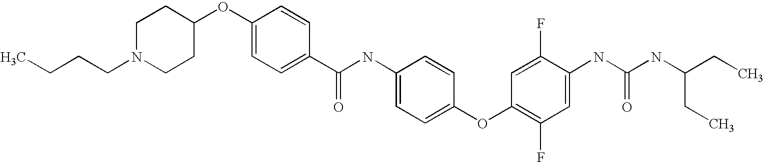 | 609 | 609 |
| 265 | 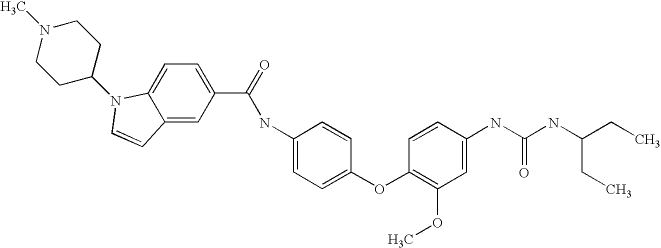 | 584 | 584 |
| 266 | 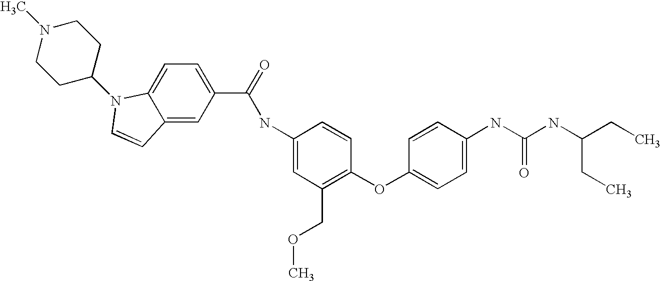 | 598 | 598 |
| 267 | 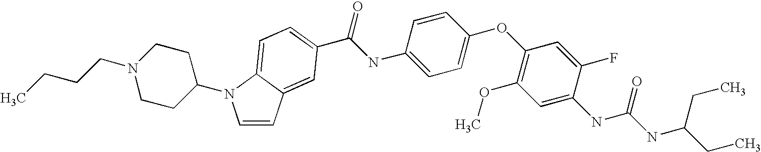 | 644 | 644 |
| 268 | 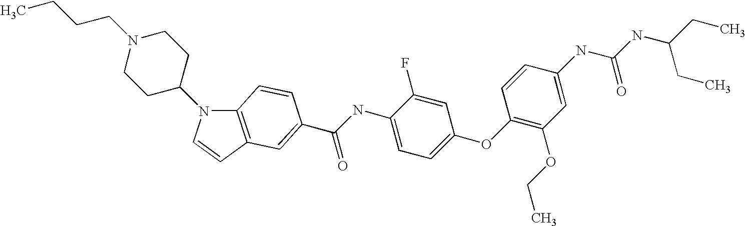 | 658 | 658 |
| 269 | 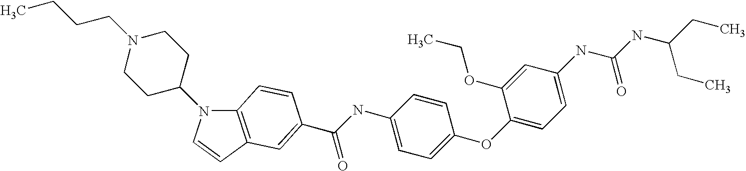 | 640 | 640 |
| 270 | 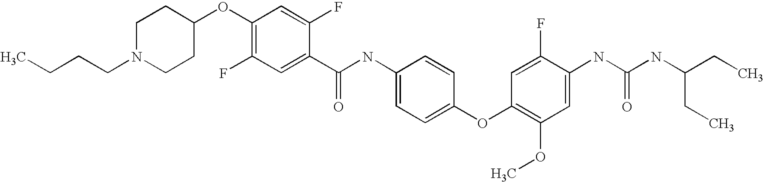 | 657 | 657 |
| 271 | 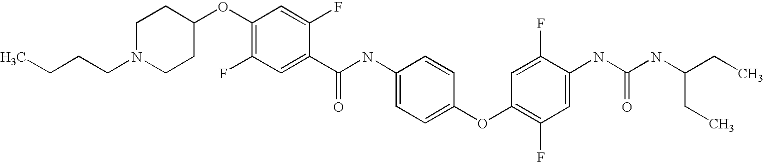 | 645 | 645 |
| 272 | 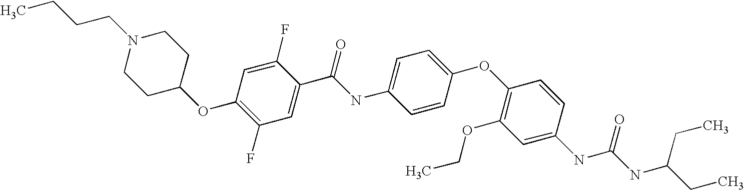 | 653 | 653 |
| 273 | 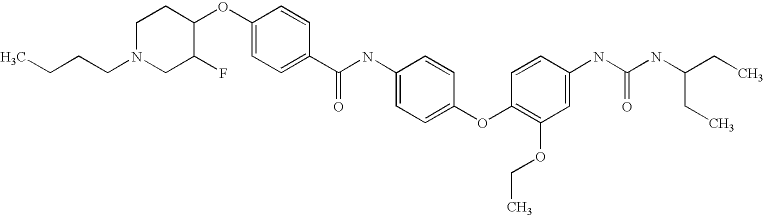 | 635 | 635 |
| 274 | 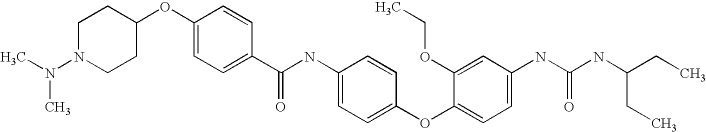 | 604 | 604 |
| 275 | 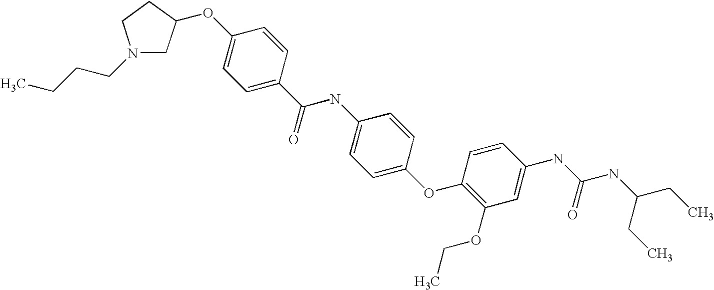 | 603 | 603 |
| 276 | 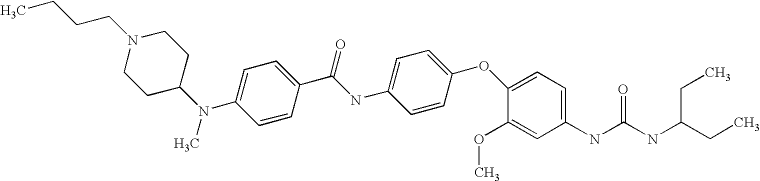 | 616 | 616 |
| 277 | 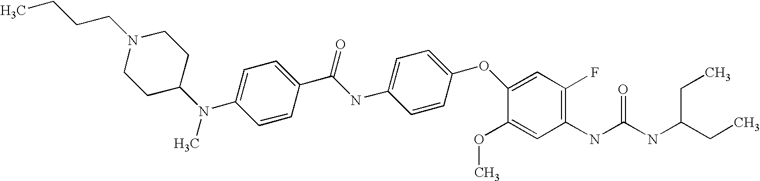 | 634 | 634 |
| 278 | 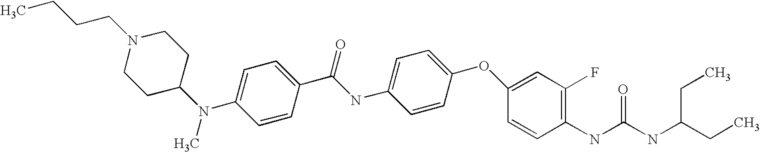 | 604 | 604 |
| 279 | 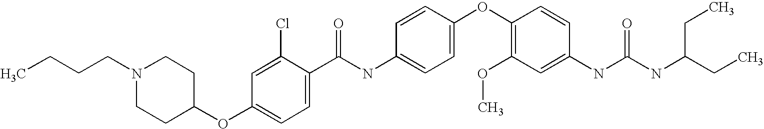 | 638 | 638 |
| 280 | 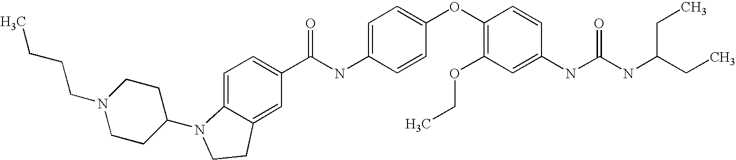 | 642 | 642 |
| 281 | 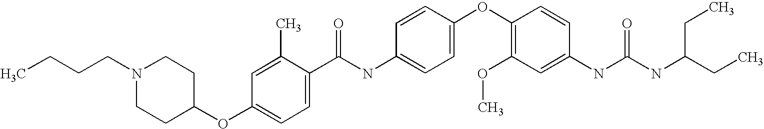 | 617 | 617 |
| 282 | 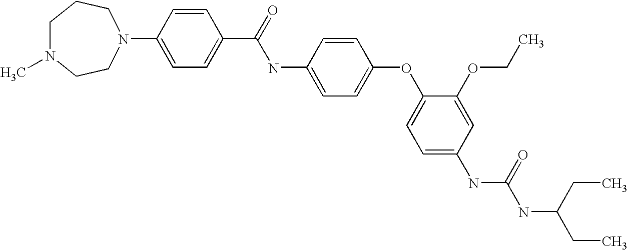 | 574 | 574 |
| 283 | 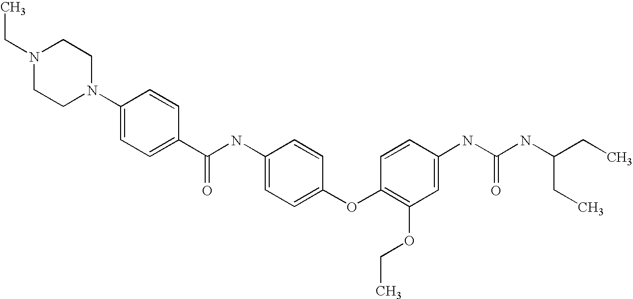 | 574 | 574 |
| 284 | 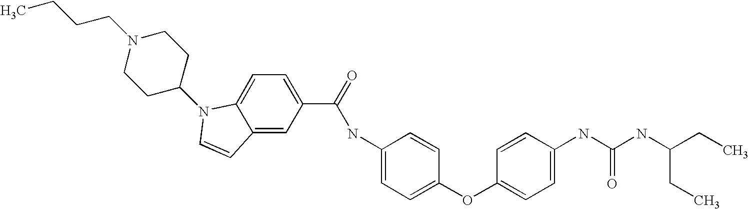 | 596 | 596 |
| 285 | 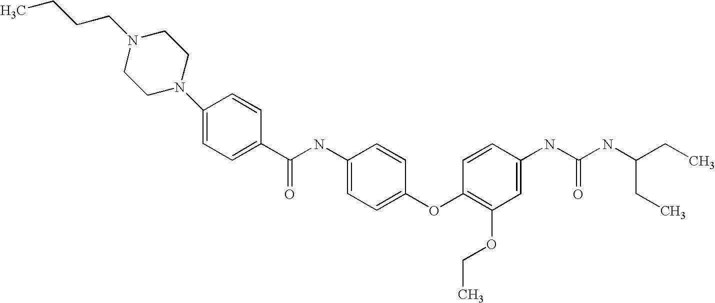 | 602 | 602 |
| 286 | 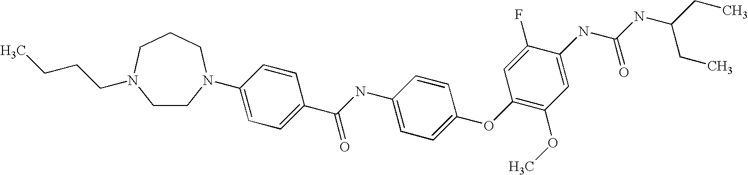 | 620 | 620 |
| 287 | 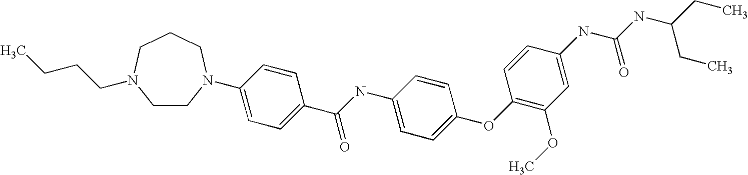 | 602 | 602 |
| 288 | 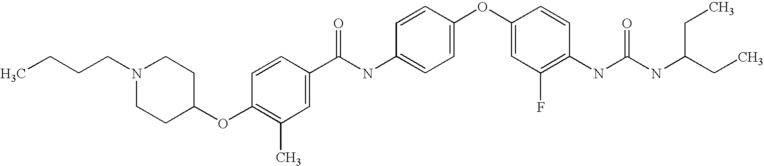 | 605 | 605 |
| 289 | 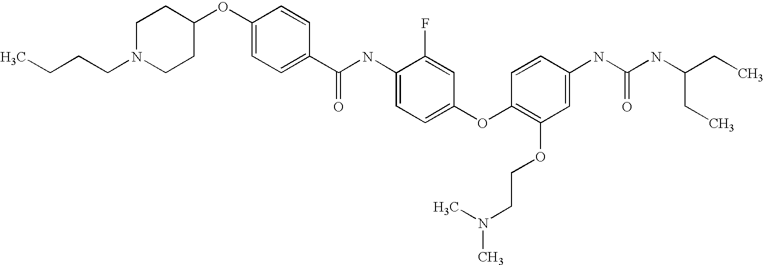 | 678 | 678 |
| 290 | 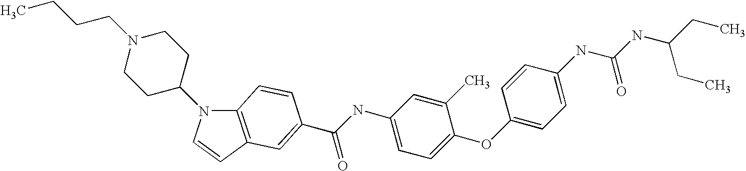 | 610 | 610 |
| 291 | 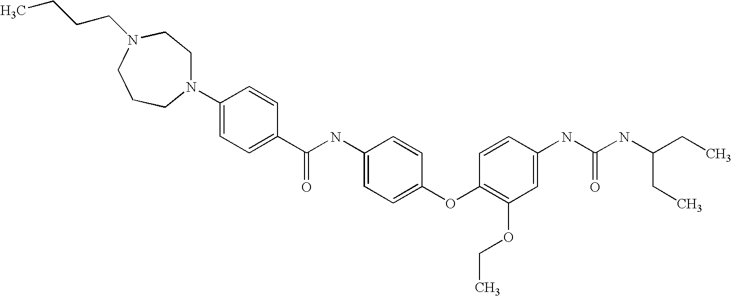 | 616 | 616 |
| 292 | 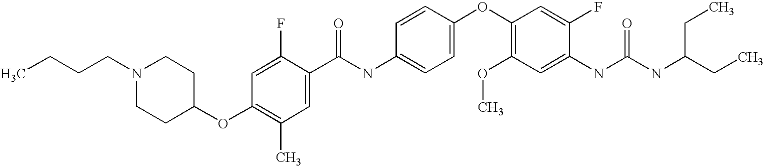 | 653 | 653 |
| 293 | 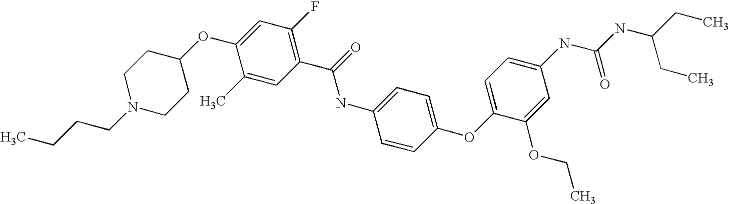 | 649 | 649 |
| 294 | 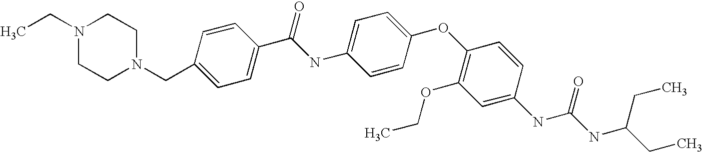 | 588 | 588 |
| 295 | 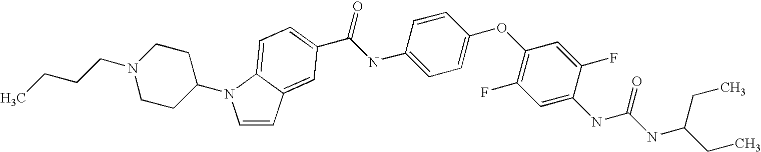 | 632 | 632 |
| 296 | 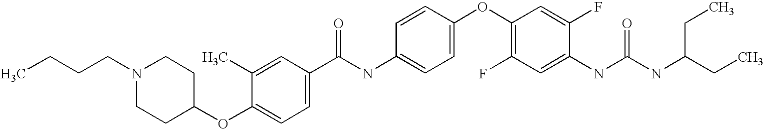 | 623 | 623 |
| 297 | 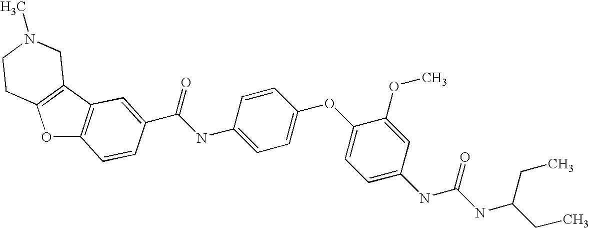 | 557 | 557 |
| 298 | 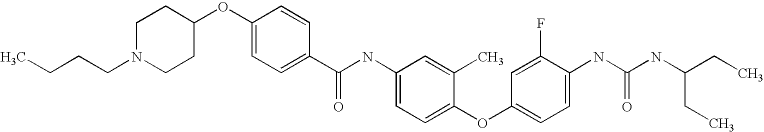 | 605 | 605 |
| 299 | 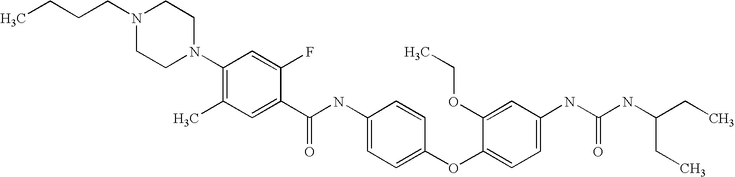 | 634 | 634 |
| 300 | 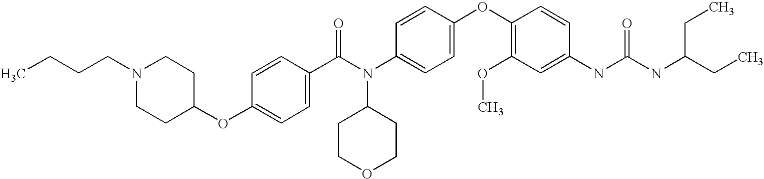 | 689 | 689 |
| 301 | 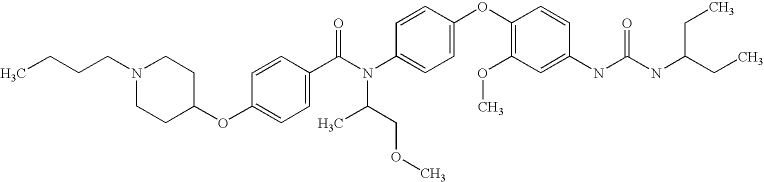 | 675 | 675 |
| 302 | 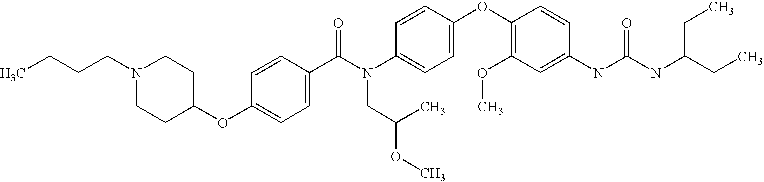 | 675 | 675 |
| 303 | 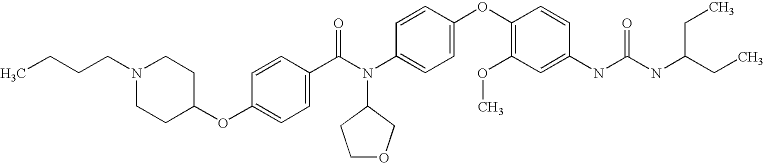 | 673 | 673 |
| 304 | 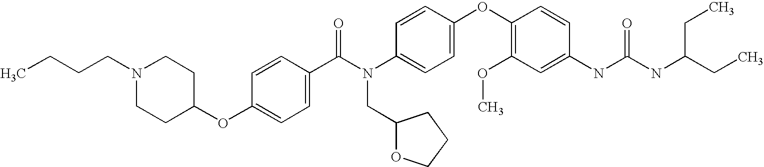 | 687 | 687 |
| 305 | 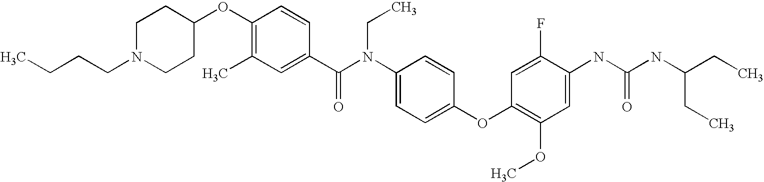 | 663 | 663 |
| 306 | 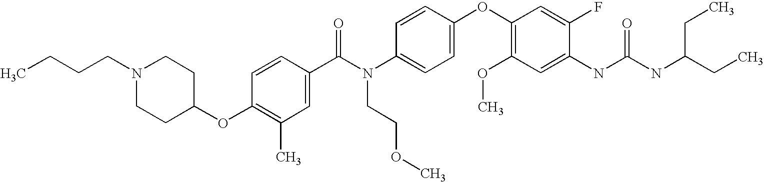 | 693 | 693 |
| 307 | 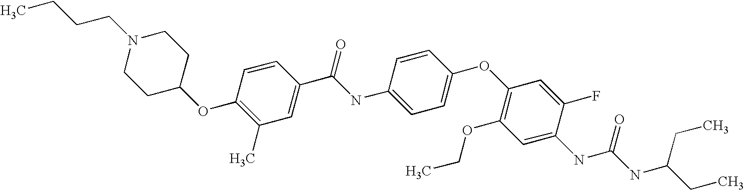 | 649 | 649 |
| 308 | 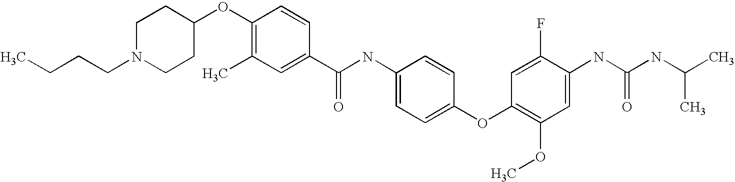 | 607 | 607 |
| 309 | 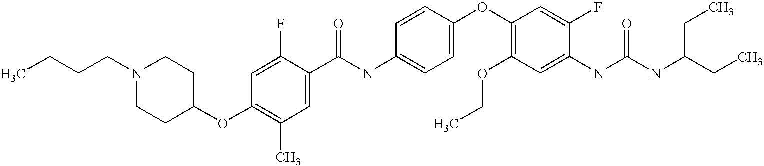 | 667 | 667 |
| 310 | 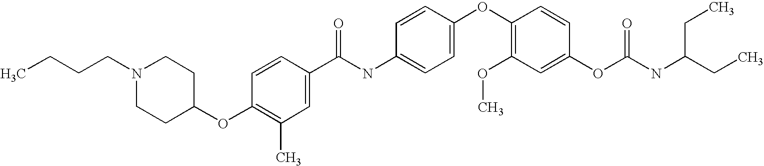 | 618 | 618 |
| 311 | 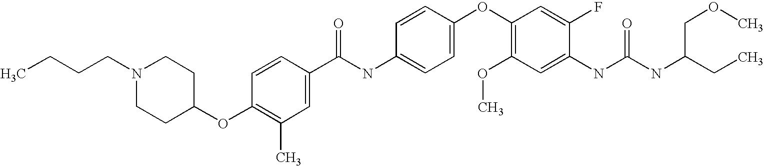 | 651 | 651 |
| 312 | 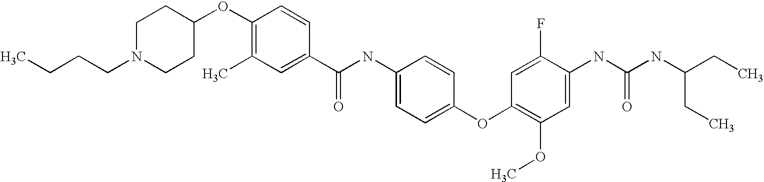 | 635 | 635 |
| 313 | 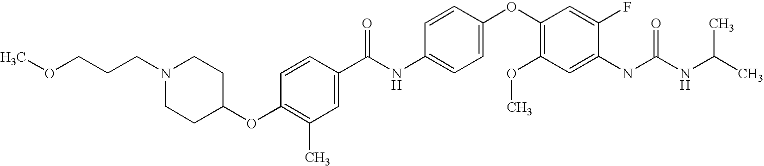 | 623 | 623 |
| 314 | 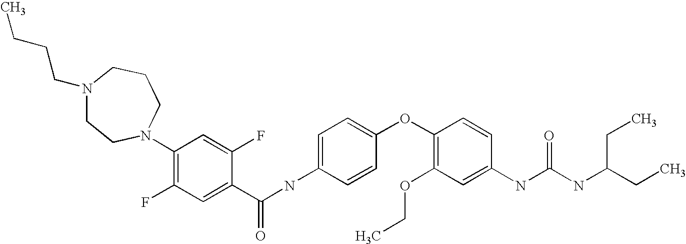 | 652 | 652 |
| 315 | 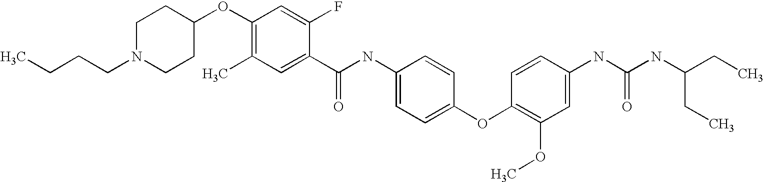 | 639 | 639 |
| 316 | 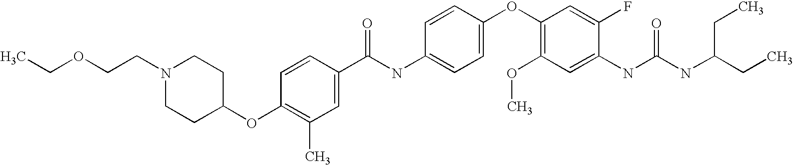 | 651 | 651 |
| 317 | 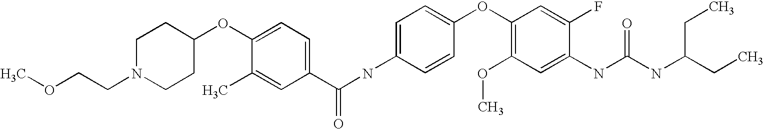 | 637 | 637 |
| 318 | 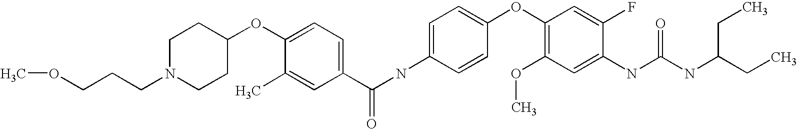 | 651 | 651 |
| 319 | 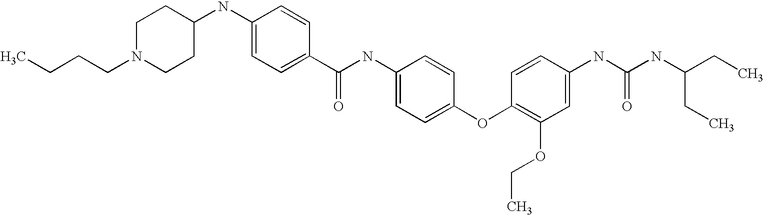 | 616 | 616 |
| 320 | 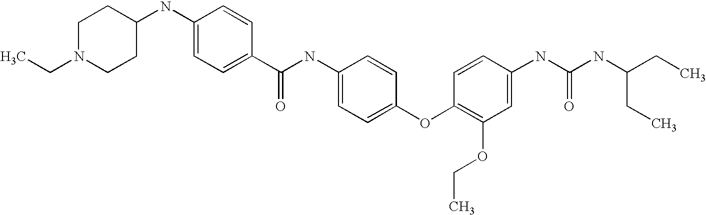 | 588 | 588 |
| 321 | 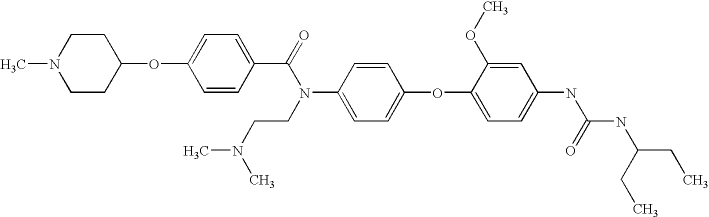 | 632 | 632 |
| 322 | 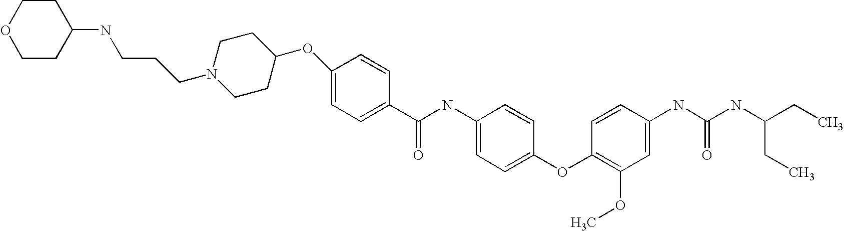 | 688 | 688 |
| 323 | 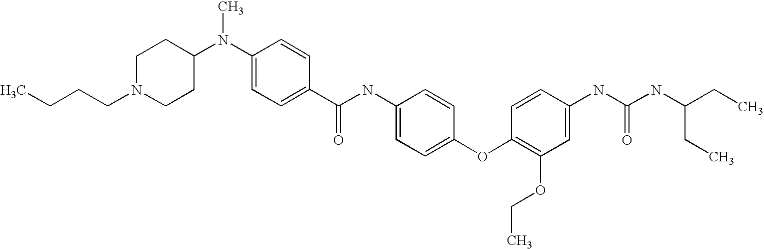 | 630 | 630 |
| 324 | 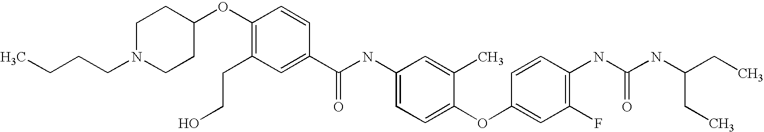 | 649 | 649 |
| 325 | 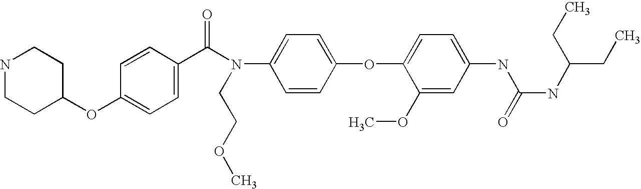 | 605 | 605 |
| 326 | 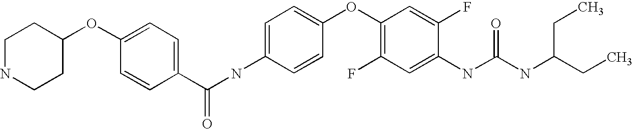 | 553 | 553 |
| 327 | 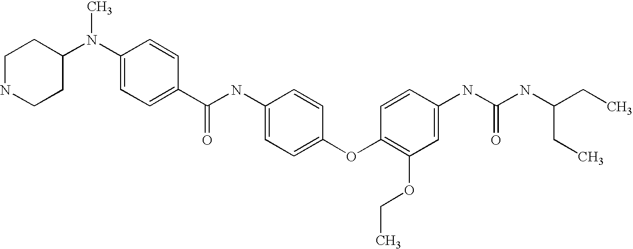 | 574 | 574 |
| 328 | 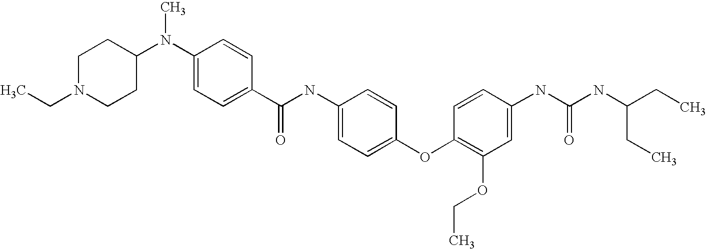 | 602 | 602 |
| 329 | 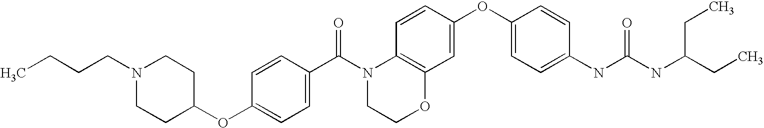 | 615 | 615 |
| 330 | 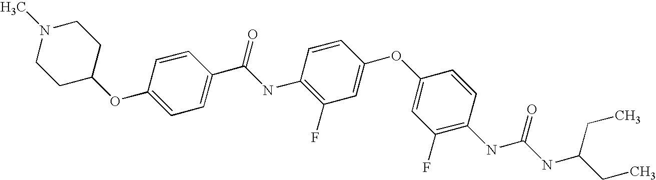 | 567 | 567 |
| 331 | 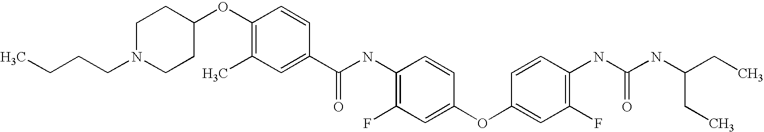 | 623 | 623 |
| 332 | 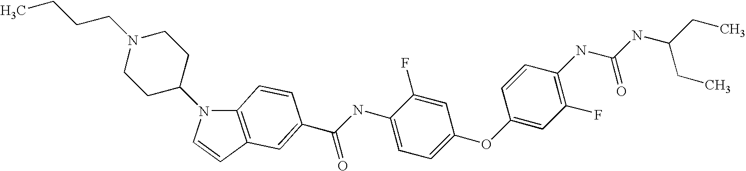 | 632 | 632 |
| 333 | 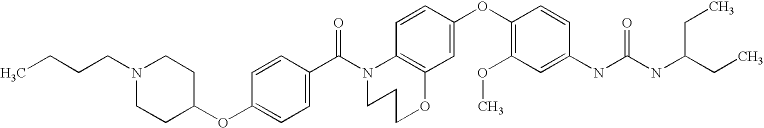 | 659 | 659 |
| 334 | 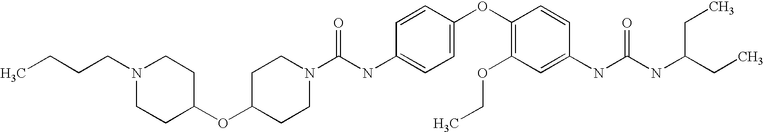 | 624 | 624 |
| 335 | 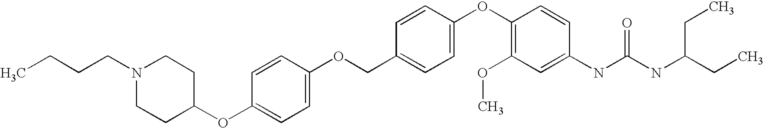 | 590 | 590 |

^{(a)} HPLC/MS analyses were conducted on Hewlett-Packard 1100 HPLC/Finnigan MAT TSQ 7000 triple-quadrupole mass spectrometer, using a Keystone Scientific column, Prism RPN C12 2 × 20 mm for separation and a binary gradient for elution with 100% solvent A to 100% solvent B in 4.1 min, and plateau of 1 min at 100% solvent B, at a flow rate of 0.3 ml/min, solvent A being a 13.3 mM ammonium formiate/6.7 mM formic acid solution in water, and solvent B being a mixture of 6 mM ammonium formiate/3 mM formic acid in water/ACN (10:90 v/v). Detection of the molecular ion of the products was conducted using the ESI⁺ technique.

Characterization of Interactions with NPY Receptors and of In Vivo Effect
1/ Characterization of Interactions with the NPY Y1 Receptor

Cell Culture

The SK-N-MC cells (ATCC HBT10) are cultured at 37° C. in MEM medium (minimum essential medium) free of phenol red (Invitrogen ref. 04194565M) containing 10% foetal calf serum (Invitrogen ref. 10270-106), 1% non-essential aminoacids (Invitrogen ref. 11140-035), 1% sodium pyruvate (Invitrogen ref. 11360-039), 1% glutamine (Invitrogen ref. 25030-032), 100 IU/ml of penicillin and 100 μg/ml of streptomycin (Invitrogen ref. 15140-122) in a humid atmosphere containing 5% CO₂.

Preparation of the Cell Suspension

After aspirating the culture medium, the cells are washed with a phosphate buffer pH 7.4 (Invitrogen ref. 14190-094), then lifted with a Versene solution (Invitrogen, ref 15040-033). The cells are centrifuged at 500×g for 10 minutes at 4° C. then resuspended in a freeze buffer pH 7.4 containing 50 mM HEPES (N-2-hydroxyethylpiperazine-N′-2-ethanesulfonic acid), 145 mM sodium chloride, 2.6 mM calcium chloride, 1 mM magnesium chloride, 10 mM glucose, and 1 mg/ml bovine albumin. The cell suspension is aliquoted into twenty million cells per milliliter of buffer and stored at −70° C.

Binding Test to the NPY Y1 Receptor

The cell suspension is incubated 2 hours at 37° C. in an incubation buffer pH 7.4 containing 50 mM HEPES, 2.5 mM calcium chloride, 1 mM magnesium chloride, 0.025% sodium azide, 1 mg/ml bovine albumin and 25 pM [¹²⁵I]-PYY (Perkin Elmer, NEX341). The reaction is halted by filtering through a GF/B filter pre-treated with 0.3% PEI, and washed three times with 1 ml of 50 mM TRIS buffer [tris(hydroxymethyl)aminomethane]/HCl, pH 7.4. The radioactivity deposited on the filter is measured by liquid scintillation count (TopCount, Packard). Non-specific binding is determined in the presence of 1 μM NPY (Bachem, H3322). Results are expressed as IC₅₀ values in nM calculated by non-linear regression with 4 parameters.

cAMP Measurement Test

The SK-N-MC cells are cultured in 96-well plates. After aspirating the culture medium, the cells are washed with a phosphate buffer pH 7.4 (Invitrogen ref. 14190-094), then lifted with a Versene solution (Invitrogen, ref 15040-033). The cells are centrifuged at 500×g for 10 minutes at 4° C. They are resuspended in a stimulation buffer containing isobutyl-methyl-xanthine in sufficient concentration to inhibit the phosphodiesterases (Flashplate kit, Perkin Elmer). The tested compounds are added 10 minutes before depositing the NPY (Bachem, H3322) in variable concentration, then the 300 nM forskoline (Sigma, F6886). The cells are in cubated 1 hour at ambient temperature to allow cAMP production whose levels are measured using the Flashplate method after 2 hours incubation with the cAMP tracer [¹²⁵I]. The results are expressed in pA2 form observing the displacement of NPY dose-effect curves in the absence and presence of increasing concentrations of test compound [Schild, 1949, pAx and competitive drug antagonism, Br. J. Pharmacol., 4, 277-280].

The compounds of the present invention are antagonists of the NPY Y1 receptor. The results in the following tables are given by way of example:

| Example no. | IC₅₀ Y1 (nM) | pA2 Y1 |
|---|---|---|
| 2 | 15.0 | 8.00 |
| 40 | 1.7 | 8.65 |
| 103 | 1.80 | 8.20 |

2/ Characterization of Interactions with the NPY Y2, Y4 and Y5 Receptors
A/ Characterization of Interactions with the NPY Y2 Receptor

Cell Culture:

The KAN.TS cells (Amersham RPNQ0081) are cultured at 37° C. in DMEM Glutamax medium (Life Technology ref. 61965026) containing 15% foetal calf serum (Invitrogen), 1% L-Glutamine (Invitrogen ref.250300-032), 50 IU/ml penicillin and 50 μg/ml of streptomycin (Invitrogen ref. 15070022) in a humid atmosphere containing 5% CO₂.

Preparation of the Cell Suspension:

After aspirating the culture medium, the cells are washed with phosphate buffer pH 7.4 (Sigma ref. D5652), then lifted with a solution of PBS⁻, 0.5 mM EDTA (ethylenediaminetetraacetic acid) (Sigma ref ED 2SS). The cells are centrifuged at 1500 rpm for 10 min at 4° C. then resuspended in a freeze buffer pH 7.4 containing 50 mM HEPES, 145 mM sodium chloride, 2.6 mM calcium chloride, 1 mM magnesium chloride, 10 mM glucose, and 1 mg/ml bovine albumin, 0.25 mg/ml bacitracin, 25 μg/ml aprotinine and 25 μg/ml leupeptine. The cells are counted and centrifuged at 1500 rpm for 10 min then resuspended in the freeze buffer and aliquoted into ten million cells per millilitre of freeze buffer, and stored at −70° C.

Binding Test to the NPY Y2 Receptor

The cell suspension with 25000 cells/ml is incubated 1 h at 37° C. in an incubation buffer pH 7.4 containing 50 mM HEPES/NaOH, 2.5 mM calcium chloride, 1 mM magnesium chloride, 0.025% sodium azide, 1 mg/ml bovine albumin and 15 pM of [¹²⁵I]-PYY (Perkin Elmer, NEX341). The reaction is halted by filtering through a GF/B filter pre-treated with 0.3% PEI, and washed three times with 1 ml of 50 mM TRIS/HCl buffer, pH 7.4. The radioactivity deposited on the filter is measured by liquid scintillation count (TopCount, Packard). Non-specific binding is determined in the presence of 1 μM NPY (Bachem, H3322). The resultats are expressed as inhibition percentage of specific binding in the presence of 10 μM or 1 μM of compound, or IC₅₀ in nM calculated by non-linear regression.

### B/ Binding Tests to Tire NPY Y4 and Y5 Receptors

CHO-Y4H and CHO-Y5H Cell Cultures

The CHO cells expressing either the Y4 or the Y5 human recombinant receptor are cultured in DMEM medium to which is added 5% dialysed foetal calf serum, 10 mM Hepes buffer and 0.8 g/l sodium bicarbonate. They are lifted from their support using a 36 mM citrate buffer without trypsin and without EDTA, and washed in PBS buffer free of Ca²⁺ and of Mg²⁺. The cell residues are stored at (−80° C.) until fractioning.

Membrane Preparation

The cell residue is redissolved in 10 mM TRIS buffer, 3 mM MgCl₂, pH 7.4 and separated with polytron. After centrifuging at 20 000×g the residue is redissolved in this same buffer, potter separated and aliquoted for storage in liquid nitrogen to around 5 mg/ml proteins.

Binding Test to the NPY Y4 Receptor

Approximately 80 μg of membranes of CHO cells having stable expression of the human Y4 receptor are incubated for 60 min at 30° C. in 200 μl Krebs-Ringer buffer (pH 7.4) containing 20 mM Hepes, 1% bovine serum albumin, 0.25 mg/ml bacitracin and 0.1 nM of [¹²⁵I]-human PP (Pancreatic Polypeptide, Perkin Elmer, NEX 315). The reaction is halted by filtering through Wathman GF/C filters and washing with 3 times 4 ml of buffer at 4° C. The radioactivity deposited on the filter is counted with a gamma counter (Whizard 1470, Wallac, Perkin Elmer). Non-specific binding is determined in the presence of 0.3 μM of human PP (Neosystem, SC104). The results are expressed as percentage inhibition of specific binding in the presence of 10 or 1 μM of compound, or IC₅₀ in nM calculated by non-linear regression.

Binding Test to the NPY Y5 Receptor

Approximately 80 μg of membranes of CHO cells having stable expression of the human Y5 receptor are incubated for 60 min at 30° C. in 200 μl Krebs-Ringer buffer (pH 7.4) containing 20 mM Hepes, 1% bovine serum albumin, 0.25 mg/ml bacitracin and 0.1 nM of [¹²⁵I]-human PYY (Perkin Elmer, NEX 341). The reaction is halted by filtering through Wathman GF/C filters and washing with 3 times 4 ml of buffer at 4° C. The radioactivity deposited on the filter is counted with a gamma counter (Whizard 1470, Wallac, Perkin Elmer). Non-specific binding is determined in the presence of 0.3 μM porcine NPY (Neosystem, SC116). The results are expressed as percentage inhibition of specific binding in the presence of 10 μM or 1 μM of compound, or IC₅₀ in nM calculated by non-linear regression.

The compounds of the present invention are more particularly selective antagonists of the NPY Y1 receptor. The results in the following table are given by way of example.

| | IC₅₀ (nM) (% Inh at 10 μM) | | | |
|---|---|---|---|---|
| Example no. | Y1 | Y2 | Y4 | Y5 |
| 103 | 1.80 nM | >10000 nM | 2620 nM | >10000 nM |
| | | (10%) | | (0%) |

### 3/ Characterization of the In Vivo Effect

### A/ Food Intake by Fasting Mice

The day before the experiment at 16 h, male OF1 mice (Charles River, France) with body weight varying between 20 and 25 g, are left to fast in individual cages with unlimited drink water. On the day of the experiment, at 9 h30±15 min, a control batch of 10 mice was given the solvent (5% DMSO, Merck, 1.02931.1000, 5% cremophor EL, Sigma C-5135, physiological saline solution to complete to volume) via intra peritoneal route or per os in a volume of 10 ml/kg, and the other batches of 10 mice were given the products to be tested dissolved in the solvent (10 or 30 mg/kg in a volume of 10 ml/kg ip ou po). Individual feed troughs filled with food (A04, UAR, France) were weighed then placed in the cages, exactly 30 min or 60.min after treating the mice ip or per os, respectively. The feed troughs were then weighed 1 h, 2 h, 3 h, 4 h and when applicable 6 h and 24 h after placing the feed troughs in the cages. Food consumptions are expressed in grams, as a mean±standard error (S.E.M). (n=10). Statistical analysis used ANOVA followed by Dunnett's multiple comparison test. The level of significance is obtained for p<0.05.

The results in the following table are given by way of example.

Accumulated inhibition
of food intake after ip
administering of 30 mg/kg

| Example no. | 0-1 h | 0-2 h | 0-3 h | 0-4 h |
|---|---|---|---|---|
| 312 | 42%* | 21% | 33%** | 27%* |

*p < 0.05 and
**p < 0.01 vs control animals

### B/ Measurement of Blood Pressure in Anaesthetized Rats

CD® male rats (Charles River, France) of body weight between 250 and 300 g, were anaesthetized with 150 mg/kg i.p. Inactin® (Sigma, T133) and tracheotomised. The jugular vein and carotid were catheterized with an Intramedic PE50 catheter to allow administering of the compounds and recording of blood pressure. Recording of blood pressure was made using a Statham P23 ID sensor coupled to a PlugSys amplifier (Hugo Sachs Elektronik) and the signal was analyzed using IOX-16™ software (EMKA Technologies, France). The compounds to be tested were dissolved in a mixture of 10% DMSO (Merck, 1.02931.1000), 5% cremophor EL (Sigma C-5135) 0.9% NaCl to complete to volume, and administered via intravenous route. (0.3 to 3 mg/kg) in the anaesthetized animals or via oral route (3 to 30 mg/kg) 60 minutes before inducing anaethesia. A control group only receiving the vehicle (in a volume of 1 or 5 mL/kg) was included in each study. Hypertension was induced via i.v. bolus at regular intervals of 5 μg/kg [Leu³¹, Pro³⁴]NPY (Neosystem, SC935). Variations in pressure were expressed in mmHg, as a mean±standard error (S.E.M) (n=4-11). Statistical analysis had recourse to ANOVA followed by Dunnett's multiple comparison test. The level of significance was obtained for p<0.05.

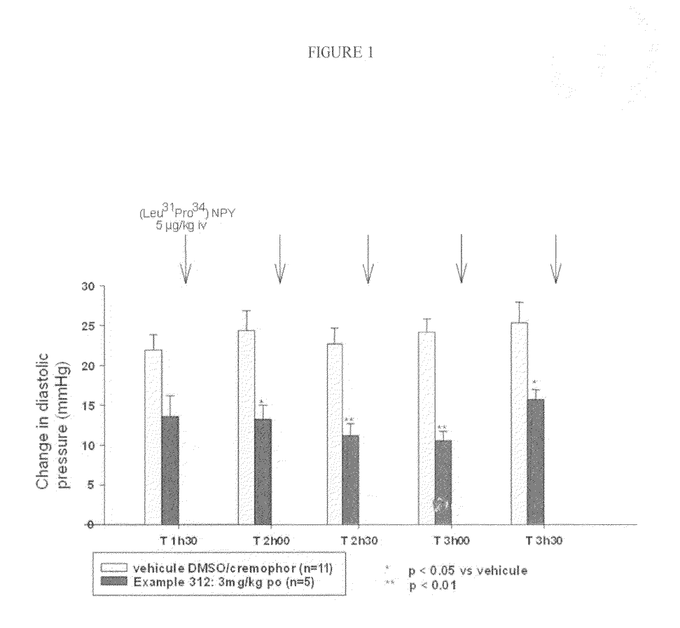

The results given in FIG. 1 are given by way of example.

1-23. (canceled)

24. A compound having the following formula (I): 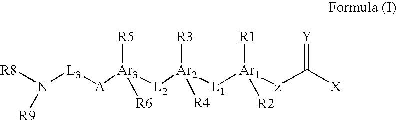
wherein:
X represents a N—(C1-C6)alkylamino group, optionally substituted by a (C1-C3)alkoxy(C1-C3)alkyl group; a N,N—(C1-C6)dialkylamino(C1-C3)alkyl group,
or X is a group of the hydrazino type, represented below: 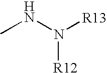
wherein R12 and R13, the same or different, represent a hydrogen atom or a (C1-C6)alkyl radical, or R12 and R13 together with the nitrogen atom to which they are attached, form a nitrogen-containing heterocycle which is aziridine, azetidine, pyrrolidine, piperidine or homopiperidine,
Z represents an oxygen atom or an —NH— radical,
Ar1 represents phenyl,
Y represents the oxygen or sulfur atom,
or Y represents a nitrogen atom and in this case, together with Z and the phenyl to which Z is attached, it forms a heterocycle which is benzimidazole or benzoxazole,
R1 and R2, the same or different, represent a hydrogen atom; a halogen atom; a hydroxyl group; a (C1-C6)alkyl, (C1-C6)alkoxy, hydroxy(C1-C3)alkyl, (C1-C6)alkoxy(C1-C3)alkyl, (C1-C3)alkoxy(C2-C3)alkoxy, hydroxy(C2-C3)alkoxy, amino(C2-C3)alkoxy, N—(C1,C3)alkylamino(C2-C3)alkoxy, N,N—(C1-C3)dialkylamino(C2-C3)alkoxy, trifluoromethyl, trifluoromethoxy, aminocarbonyl, N—(C1-C6)alkylaminocarbonyl, N,N—(C1-C6)dialkylaminocarbonyl, aminocarbonyl(C1-C3)alkyl, N—(C1-C6)alkylaminocarbonyl(C1-C3)alkyl, N,N—(C1-C6)dialkylaminocarbonyl(C1-C3)alkyl, (C1-C6)alkoxycarbonyl or (C1-C6)alkoxycarbonyl(C1-C3)alkyl radical,
L1 represents an oxygen atom, a sulfur atom or a (C1-C3)alkylene group,
Ar2 represents an aryl, heteroaryl or heterocycle group which is phenyl, thiazole, indole, benzofuran, benzoxazole, benzimidazole, 2,3-dihydrobenzofuran, or 3H-quinazolin-4-one,
R3 and R4, the same or different, represent a hydrogen atom; a halogen atom; a hydroxyl group; a (C1-C6)alkyl, (C1-C6)alkoxy, hydroxy(C1-C3)alkyl, (C1-C6)alkoxy(C1-C3)alkyl, (C1-C3)alkoxy(C2-C3)alkoxy, hydroxy(C2-C3)alkoxy, amino(C2-C3)alkoxy, N—(C1-C3)alkylamino(C2-C3)alkoxy, N,N—(C1-C3)dialkylamino(C2-C3)alkoxy, trifluoromethyl or trifluoromethoxy radical,
R1 and R3, together and with Ar1, Ar2 and L1, can also form a tricycle and in this case R1 and R3 together represent a (C1-C3)alkylene group, with L1 representing an oxygen or sulfur atom and Ar2 a phenyl,
when Ar2 is a phenyl or a thiazole, L2 represents one of the groups below: 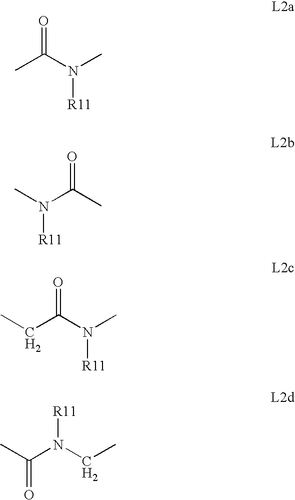
wherein:
R11 represents a hydrogen atom; a (C1-C6)alkyl radical, optionally mono or polyfluorinated, optionally substituted by a heterocycle such as tetrahydrofuran or tetrahydropyran; a (C3-C10)cycloalkyl radical; a hydroxy(C2-C6)alkyl group; (C1-C6)alkoxy(C2-C6)alkyl group; amino(C2-C6)alkyl group; N—(C1-C6)alkylamino(C2-C6)alkyl group; N,N—(C1-C6)dialkylamino(C2-C6)alkyl group; or a heterocycle which is tetrahydrofuran or tetrahydropyran;
for L2a, L2c and L2d, R11 can also, together with Ar2 which in this case represents a phenyl group, and with the nitrogen to which it is attached, form a heterocycle which is indoline; isoindoline; tetrahydroisoquinoleine; tetrahydroquinoleine; 3,4-dihydro-2H-benzo[1,4]oxazine; 6,7,8,9-tetrahydro-5-oxa-9-aza-benzocycloheptene or 1,2,3,5-tetrahydro-benzo[e][1,4]oxazepine;
or for L2b, R11 can also together with Ar3, which in this case represents a phenyl group, and with the nitrogen to which it is attached, form a heterocycle which is indoline; tetrahydroquinoleine; 3,4-dihydro-2H-benzo[1,4]oxazine; 6,7,8,9-tetrahydro-5-oxa-9-aza-benzocycloheptene or 1,2,3,5-tetrahydro-benzo[e][1,4]oxazepine;
additionally, for L2a, L2c and L2d, R11 can, together with Ar3 which in this case represents a phenyl group, and with the nitrogen to which it is attached, form a heterocycle which is 1,3-dihydro-indol-2-one; 2,3-dihydro-isoindol-1-one; 1,4-dihydro-2H-isoquinolin-3-one or 3,4-dihydro-2H-quinolin-1-one;
or for L2b, R11 can, together with Ar2 which in this case represents a phenyl group, and with the nitrogen to which it is attached, form a heterocycle which is 2,3-dihydro-isoindol-1-one or 3,dihydro-2H-isoquinolin-1-one;
or L2 represents a methyleneoxy or oxymethylene radical,
or L2 represents a simple bond with Ar2 representing a phenyl, indole, benzofuran, benzoxazole, benzimidazole, or 3H-quinazolinone group,
or L2 represents a simple bond, with Ar2 representing a phenyl group and Ar3 representing an indole, benzofuran, benzoxazole, benzimidazole, 2,3-dihydro-benzofuran or 3H-quinazolinone group,
Ar3 represents a heteroaryl, aryl or heterocyclic group which is phenyl, indole, benzofuran, benzoxazole, benzimidazole, 2,3-dihydro-benzofuran, or piperidine, Ar3 and Ar2 not being heteroaryl or heterocyclic groups simultaneously when L2 is a simple bond,
R5 and R6, the same or different, represent a hydrogen atom; a halogen atom; a hydroxyl or trifluoromethyl group; a (C1-C6)alkyl, (C1-C6)alkoxy, hydroxy(C1-C3)alkyl, (C1-C6)alkoxy(C1-C3)alkyl, (C1-C3)alkylcarbonyl, (C1-C3)alkoxy(C2-C3)alkoxy, hydroxy(C2-C3)alkoxy, amino(C2-C3)alkoxy, N—(C1-C3)alkylamino(C2-C3)alkoxy or N,N—(C1-C3)dialkylamino(C2-C3)alkoxy radical,
A represents a simple bond; an oxygen atom; a (C1-C3)alkylene, (C2-C3)alkylidene, (C1-C3)alkylenoxy or oxy(C1-C3)alkylene group,
Or A represents one of the groups described below: 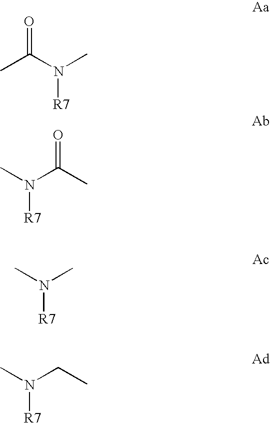
wherein:
R7 represents a hydrogen atom; a (C1-C6)alkyl or (C1-C6)alkylcarbonyl group;
additionally R7 can, together with L3 and the nitrogen atom to which R7 is attached, form a nitrogen-containing heterocycle which is piperidine, pyrrolidine, homopiperidine, pyrrolidin-2-one, piperidin-2-one or azepan-2-one;
R7 can optionally, together with Ar3 which in this case represents a phenyl group, and with the nitrogen to which it is attached, form a heterocycle which is indoline, tetrahydroquinoleine, 2,3-dihydro-isoindol-1-one or 3,4-dihydro-2H-isoquinolin-1-one,
L3 represents a (C1-C6)alkylene, (C3-C8)cycloalkylene, N—(C2-C6)alkyleneamino, (C2-C6)alkylidene, (C3-C8)cycloalkylidene, bicyclo or polycyclo(C6-C12)alkylene, bicyclo or polycyclo(C6-C12)alkylidene radical, L3 not being a methylene radical if it is directly bound both to an oxygen atom and to a nitrogen atom or to two nitrogen atoms, the afore-cited radicals optionally being substituted by one or more fluorine atoms, by one or more (C1-C3)alkyl, (C1-C3)alkoxy, hydroxy, hydroxy(C1-C3)alkyl or (C1-C3)alkoxy groups,
L3 can optionally, together with A and Ar3, form an oxygen-containing heterocycle which is 2,3-dihydrobenzofuran, benzofuran or chromane,
R8 and R9, the same or different, represent a hydrogen atom; a (C1-C6)alkyl group, optionally substituted by a phenyl radical, by a saturated oxygen- or nitrogen-containing heterocycle which is tetrahydropyran-3 or 4-yl, piperidin-3 or -4-yl, pyrrolidin-3-yl or morpholin-1-yl; a (C1-C6)alkoxy(C2-C6)alkyl group; a (C3-C8)cycloalkyl group; a (C3-C8)cycloalkyl(C1-C4)alkyl group; a saturated nitrogen- or oxygen-containing heterocycle which is tetrahydropyran-3 or 4-yl, piperidin-3 or 4-yl or pyrrolidin-3-yl; an amino, N—(C1-C6)alkylamino, N,N—(C1, C6)dialkylamino, amino(C2-C6)alkyl, N—(C1-C4)alkylamino(C2-C6)alkyl, N,N(C1-C4)dialkylamino(C2-C6)alkyl, N,N—(C1-C4)dialkylamino(C1-C6)alkylcarbonyl, tetrahydropyran-4-yl-amino(C2-C6)alkyl, hydroxy(C2-C6)alkyl, (C1-C4)alkoxy(C2-C6)alkyl, hydroxycarbonyl(C1-C3)alkyl, (C1-C6)alkoxycarbonyl(C1-C3)alkyl or (C1-C3)alkylcarbonyloxy(C2-C6)alkyl radical, the afore-cited groups optionally being substituted by one or more fluorine atoms,
R8 and R9, together and with the nitrogen atom to which they are attached, can form a nitrogen-containing mono- or polycyclic heterocycle which is aziridine, azetidine, pyrrolidine, piperidine, piperazine, homopiperazine, [1,5]diazocane, homopiperidine, morpholine, 2,7-diaza-spiro[4.4]nonane, octahydro-pyrrolo[3,4-c]pyrrole, or octahydro-pyrrolo[3,2-b]pyrrole, optionally substituted by one or more fluorine atoms, by one or more hydroxyl, hydroxy(C1-C6)alkyl, (C1-C6)alkyl, (C1-C6)alkoxy, amino(C1-C6)alkyl, N—(C1-C4)alkylamino(C1-C6)alkyl, N,N—(C1-C4)dialkylamino(C1-C6)alkyl, (C1-C4)alkoxy(C1-C6)alkyl, hydroxycarbonyl(C1-C3)alkyl, (C1-C6)alkoxycarbonyl(C1-C3)alkyl, (C1-C3)alkylcarbonyloxy(C1-C6)alkyl or mono or polyfluoro(C1-C6)alkyl radicals,
R8 and/or R9, together with L3 and the nitrogen atom to which they are attached can form a mono- or polycyclic nitrogen-containing heterocycle, saturated or unsaturated, which is pyrrolidine, piperidine, homopiperidine, 8-azabicyclo[3.2.1]octane, 2-aza-bicyclo[2.2.2]octane, 2-aza-bicyclo[2.2.1]heptane, 7-aza-bicyclo[2.2.1]heptane, 1,2,3,6-tetrahydro pyridine, optionally substituted by one or more fluorine atoms, by one or more hydroxyl, hydroxy(C1-C6)alkyl, (C1-C6)alkyl, (C1-C6)alkoxy, amino(C1-C6)alkyl, N—(C1-C4)alkylamino(C1-C6)alkyl, N,N—(C1-C4)dialkylamino(C1-C6)alkyl, (C1-C4)alkoxy(C1-C6)alkyl, hydroxycarbonyl(C1-C3)alkyl, (C1-C6)alkoxycarbonyl(C1-C3)alkyl, (C1-C3)alkylcarbonyloxy(C1-C6)alkyl or mono or polyfluoro(C1-C6)alkyl radicals; for the particular case in which L3 together with A and Ar3 forms an oxygen-containing heterocycle, and at the same time R8 and/or R9 together with L3 and with the nitrogen atom to which they are attached form a nitrogen-containing heterocycle, the whole forms a polycycle which is 1,2,3,4-tetrahydro-benzo[4,5]furo[3,2-c]pyridine or 1,2,3,4,4a,9b-hexahydro-benzo[4,5]furo[3,2-c]pyridine, or else a polycycle of the formula below: 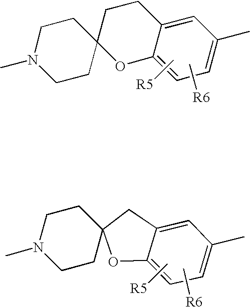
when A represents one of the groups Aa, Ab, Ac or Ad, R8 and/or R9 may optionally, together with R7, L3 and the nitrogen atom to which R8 and R9 are attached, form a mono or polycyclic nitrogen-containing heterocycle which is piperazine, homopiperazine, [1,5]diazocane, 2,7-diaza-spiro[4.4]nonane, octahydro-pyrrolo[3,4-c]pyrrole, octahydro-pyrrolo[3,2-b]pyrrole, piperazin-2-one, [1,4]diazepan-5- or -2-one, or [1,5]diazocan-2-one,
the nitrogen atom attached to R8 and R9 optionally being in quaternary ammonium form, which is then found in the following form: 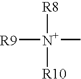
R8 and R9 being as defined above, and R10 represents a (C1-C6)alkyl group,
or one of its pharmaceutically acceptable salts, its solvates and hydrates, optical and geometric isomers, or a mixture thereof.

25. The compound of formula (I) according to claim 24, wherein at least one of the R8 and R9 groups is different from the hydrogen atom.

26. The compound of formula (I) according to claim 24, wherein R1 and R2 are simultaneously different from the hydrogen atom.

27. The compound according to claim 24, wherein Y represents an oxygen atom, Z represents an —NH— radical and X represents an N—(C1-C6)alkylamino group, optionally substituted by a (C1-C3)alkoxy(C1-C3)alkyl group.

28. The compound according to claim 24, wherein the compound has the following formula (I′):

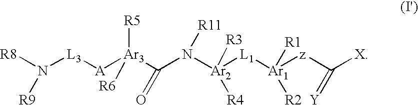

29. The compound according to claim 24, wherein A is an oxygen atom, Ar1 and Ar3 are phenyl radicals, Ar2 is a thiazole or phenyl, and X, Y, Z, L1, L3, R1 to R9 and R11 are as defined in claim 24 and the compound has the following formula (II):

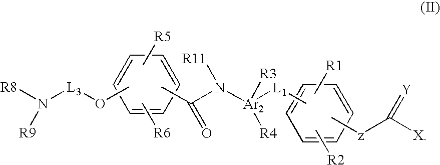

30. The compound according to claim 24, having the following formula (IIa), with Ar1 and Ar3 being 3- or 4-phenyl radicals:

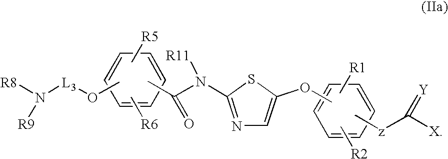

31. The compound according to claim 24, having the following formula (IIb), with Ar1, Ar2 and Ar3 being 3 or 4-phenyl radicals:

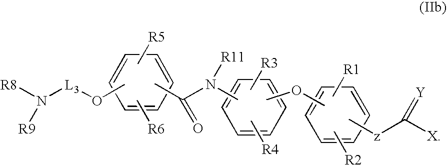

32. The compound according to claim 24, having the following formula (Ibc): 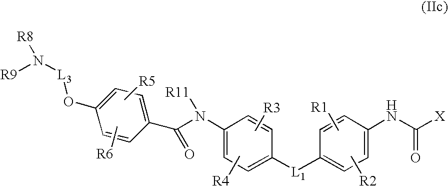
wherein:
X represents a (C1-C6)alkylamino group, optionally substituted by a (C1-C3)alkoxy(C1-C3)alkyl group, and/or
L1 is a sulfur atom or a methylene —CH₂— radical, and/or
R11 represents a hydrogen atom or a (C1-C6)alkyl radical, and/or
L3 is a (C2-C6)alkylene group, and/or
R8 and R9, together and with the nitrogen atom to which they are attached, form a nitrogen-containing heterocycle, which is piperidine,
or R9, together with L3 and with the nitrogen atom to which it is attached, forms a nitrogen-containing heterocycle, which is piperidine,
R1 to R6 are as defined in claim 1.

33. The compound according to claim 24, having the following formula (IIIa): 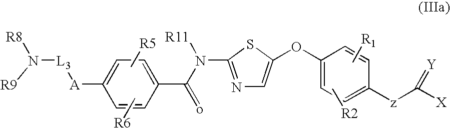
wherein the group: 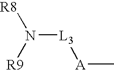
represents:

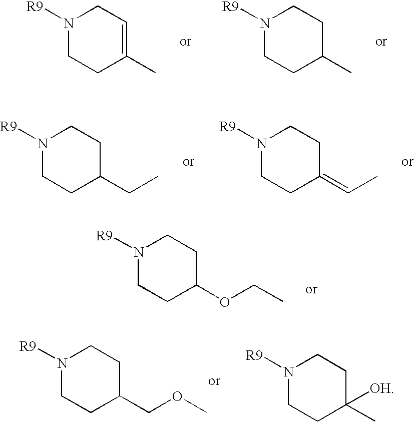

34. The compound according to claim 24, having the following formula (IIIb): 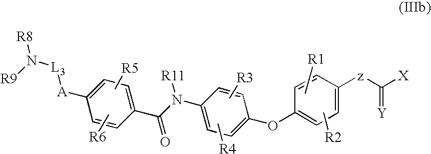
wherein the group: 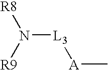
represents:

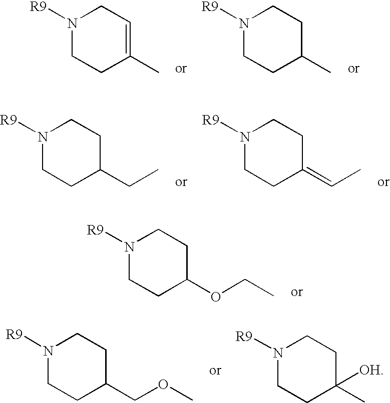

35. The compound according to claim 24, having the following formula (IIIc): 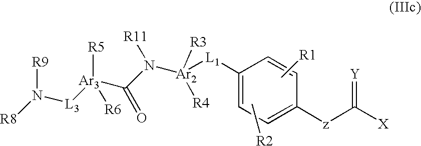
wherein:
Ar2 is a phenyl radical, or thiazole, and/or
Ar3 is an indole, benzimidazole or benzofuran group,
the group: 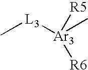
represents: 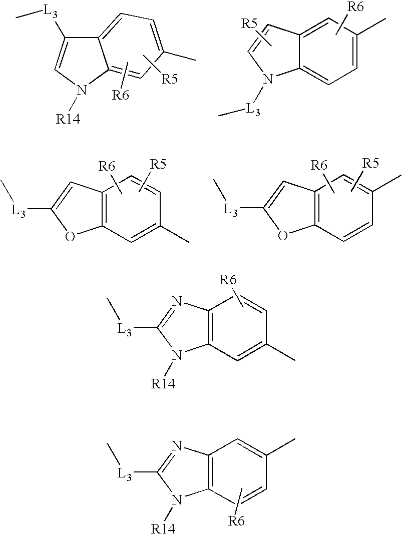
X, Y, Z, L1, L3, R1 to R11 are as defined in claim 1, and R14 is a hydrogen atom, a (C1-C6)alkyl or (C1-C3)alkoxy(C2-C6)alkyl radical.

36. The compound according to claim 24, having the following formula (IIId): 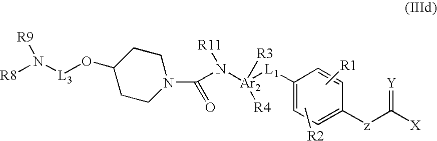
wherein:
A represents oxygen,
Ar2 is a phenyl radical, or thiazole,
Ar3 is a piperidine,
the group: 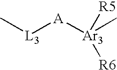
in formula (IIId) above represents: 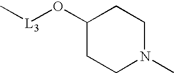
X, Y, Z, L1, L3, R1 to R4, R8, R9 and R11 are as defined in claim 24.

37. The compound according to claim 24, having the following formula (IVa): 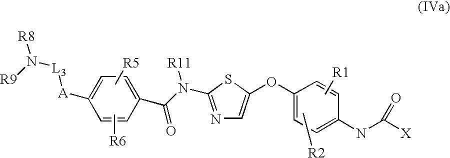
wherein:
A represents one of the groups below: 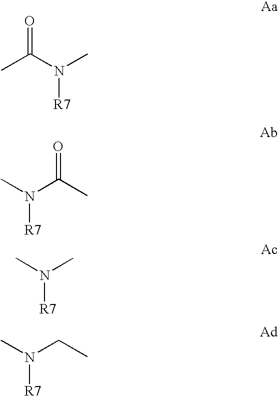
X, R1, R2, R5 to R8 and R11 are as defined in claim 24.

38. The compound according to claim 24, having the following formula (IVb): 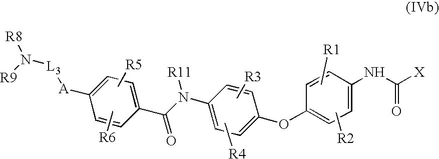
wherein:
A represents one of the groups below: 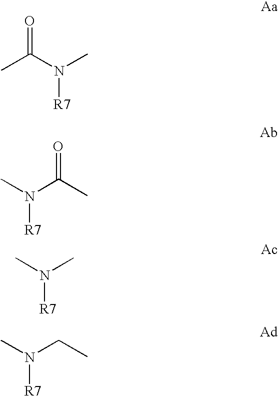
X, R1 to R8 and R11 are as defined in claim 24.

39. The compound according to claim 24, having the following formula (V): 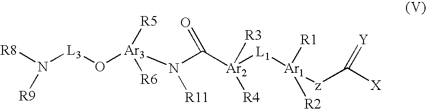
wherein Ar1, Ar2 and Ar3 are phenyl radicals; and X, Y, Z, L1, L3, R1 to R11 are as defined in claim 24.

40. The compound according to claim 24, having the following formula (VI): 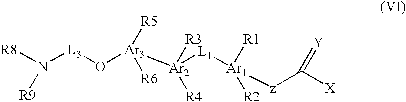
wherein Ar1 is a phenyl radical, Ar2 and Ar3 are heteroaryl, aryl or heterocyclic groups which is phenyl, indole, benzofuran, 2,3-dihydro-benzofuran, benzoxazole, or benzimidazole, Ar2 and Ar3 not being heteroaryl or heterocyclic groups simultaneously; and X, Y, Z, L1, L3 and R1 to R9 being as defined in claim 24.

41. The compound according to claim 24, which is selected from the group consisting of:
N-(5-{4-[3-(1-Ethyl-propyl)-ureido]-2-methoxymethyl-phenoxy}-thiazol-2-yl)-4-(1-isopropyl-piperidin-4-yloxy)-benzamide,
N-5-{4-[3-(4-Ethyl-propyl)-ureido]-2-methoxy-phenoxy}-thiazol-2-yl)-4-(1-isopropyl-piperidin-4-yloxy)-benzamide,
N-(5-{4-[3-(1-Ethyl-propyl)-ureido]-2-methylcarbamoylmethyl-phenoxy}-thiazol-2-yl)-4-(1-isopropyl-piperidin-4-yloxy)-benzamide,
N-{5-[4-(3-dimethylamino-ureido)-2-methylcarbamoylmethyl-phenoxy]-thiazol-2-yl}-4-(1-isopropyl-piperidin-4-yloxy)-benzamide,
N-(4-{4-[3-(1-Ethyl-propyl)-ureido]-2-methoxymethyl-phenoxy}-phenyl-4-(1-isopropyl-piperidin-4-yloxy)-benzamide,
N-(4-{4-[3-(1-Ethyl-propyl)-ureido]-2-methoxy-phenoxy}-3-methyl-phenyl)-4-(1-isopropyl-piperidin-4-yloxy)-benzamide,
N-5-{4-[3-(1-Ethyl-propyl)-ureido]-2-methoxymethyl-phenoxy}-thiazol-2-yl)-4-(1-isopropyl-piperidin-4-yloxy)-N-methyl-benzamide,
N-(4-{4-[3-(1-Ethyl-propyl)-ureido]-phenoxy}-3-methyl-phenyl)-4-(1-isopropyl-piperidin-4-yloxy)-benzamide,
N-(5-{4-[3-(1-Ethyl-propyl)-ureido]-2-methoxymethyl-phenoxy}-thiazol-2-yl)-4-(1-isopropyl-piperidin-3-ylmethoxy)-benzamide,
4-(1-Butyl-piperidin-4-yloxy-1N-5-{4-[3-(1-ethyl-propyl)-ureido]-2-methoxy-phenoxy}-thiazol-2-yl)-benzamide,
4-(1-Butyl-piperidin-4-yloxy)-N-5-{4-[3-(1-ethyl-propyl)-ureido]-2-methoxymethyl-phenoxy}-thiazol-2-yl)-benzamide,
4-(1-Butyl-piperidin-4-yloxy)-N-(5-{4-[3-(1-ethyl-propyl)-ureido]-2-fluoro-phenoxy}thiazol-2-yl)-benzamide,
4-(1-Butyl-piperidin-4-yloxy)-N-(5-{2-ethoxymethyl-4-[3-(1-ethyl-propyl)-ureido]-phenoxy}-thiazol-2-yl)-benzamide,
4-(1-Butyl-piperidin-4-yloxy)-N-(4-{2-ethoxy-4-[3-(1-ethyl-propyl)-ureido]-phenoxy}-phenyl)-benzamide,
4-(1-Butyl-piperidin-4-yloxy)-N-(4-{2-chloro-4-[3-(1-ethyl-propyl)-ureido]-phenoxy}-phenyl)-benzamide,
4-(1-Butyl-piperidin-4-yloxy)-N-{3-[4-(3-dimethylamino-ureido)-2-methoxy-phenoxy]-phenyl}-benzamide,
4-(1-Butyl-piperidin-4-yloxy)-N-(4-{4-[3-(1-ethyl-propyl)-ureido]-2-methoxy-phenoxy}-phenyl)-N-(2-hydroxy-ethyl)-benzamide,
4-(1-Butyl-piperidin-4-yloxy)-N-(4-{4-[3-(1-ethyl-propyl)-ureido]-2-methoxy-phenoxy}-phenyl)-N-(3,3,3-trifluoro-propyl)-benzamide,
4-(1-Butyl-piperidin-4-yloxy)-N-(4-{4-[3-(1-ethyl-propyl)-ureido]-2-methoxy-phenoxy}-phenyl)-N-3-methoxy-propyl)-benzamide,
4-(1-Butyl-piperidin-4-yloxy-N-(4-{4-[3-(1-ethyl-propyl)-ureido]-2-methoxy-phenoxy}-phenyl)-N-4,4,4-trifluoro-butyl)-benzamide,
4-(1-Butyl-piperidin-4-yloxy)-N-(4-{4-[3-(1-ethyl-propyl)-ureido]-2-isopropyl-phenoxy}phenyl)-benzamide,
4-(1-Butyl-piperidin-4-yloxy)-N-(4-{2-ethoxy-4-[3-(1-ethyl-propyl)-ureido]-phenoxy}-2-fluoro-phenyl)-3-methyl-benzamide,
4-(1-Butyl-piperidin-4-yloxy)-N-(4-{4-[3-(1-ethyl-propyl)-ureido]-2-methoxy-phenoxy}-phenyl)-3-methyl-benzamide,
4-(1-Butyl-piperidin-4-yloxy)-N-(4-{4-[3-(1-ethyl-propyl)-ureido]-2-methoxy-phenoxy}phenyl)-3-methoxy-benzamide,
4-(1-Butyl-piperidin-4-yloxy)-3-chloro-N-(4-{4-[3-(1-ethyl-propyl)-ureido]-2-methoxy-phenoxy}-phenyl)-benzamide,
N-(5-{4-[3-(1-Ethyl-propyl)-ureido]-2-methoxy-phenoxy}-thiazol-2-yl-4-(1-methyl-piperidin-4-yloxy)-benzamide,
N-(5-{4-[3-(1-Ethyl-propyl)-ureido]-2-methoxymethyl-phenoxyl}-thiazol-2-yl)-4-(1-isopropyl-pyrrolidin-3-yloxy)-benzamide,
N-(5-{4-[3-(1-Ethyl-propyl)-ureido]-2-methoxymethyl-phenoxy}-thiazol-2-yl)-3-(1-isopropyl-piperidin-4-yloxy)-benzamide,
N-(4-{3-[3-(1-Ethyl-propyl)-ureido]-phenoxy}-3-methyl-phenyl)-3-(1-isopropyl-piperidin-4-yloxy)-benzamide,
N-(4-{3-[3-(1-Ethyl-propyl)-ureido]-phenoxy}-3-methoxymethyl-phenyl)-3-(1-isopropyl-piperidin-4-yloxy)-benzamide,
N-(4-{5-[3-(1-Ethyl-propyl)-ureido]-2-methoxy-phenoxy}-phenyl)-3-(1-isopropyl-piperidin-4-yloxy)-benzamide,
4-(1-Benzyl-piperidin-4-yloxy)-N-(5-{4-[3-(1-ethyl-propyl)-ureido]-2-methoxy-phenoxy}-thiazol-2-yl)-benzamide,
N-(5-{4-[3-(1-Ethyl-propyl)-ureido]-2-methoxymethyl-phenoxy}-thiazol-2-yl)-4-(1-isopropyl-piperidin-3-yloxy)-benzamide,
N-(4-{3-[3-(1-Ethyl-propyl)-ureido]-phenoxy}-3-methyl-phenyl)-4-(1-isopropyl-piperidin-3-yloxy)-benzamide,
N-(4-{5-[3-(1-Ethyl-propyl)-ureido]-2-methoxy-phenoxy}-phenyl)-4-(1-isopropyl-piperidin-3-yloxy)-benzamide,
N-{3-[4-(3-dimethylamino-ureido)-2-methoxy-phenoxy]-phenyl}-4-(1-isopropyl-piperidin-3-yloxy)-benzamide,
N-(5-{4-[3-(1-Ethyl-propyl)-ureido]-2-methoxy-phenoxy}-thiazol-2-yl)-4-(1-isopropyl-piperidin-4-yloxymethyl)-benzamide,
N-(5-{4-[3-(1-Ethyl-propyl)-ureido]-2-methoxymethyl-phenoxy}-thiazol-2-yl)-3-(1-isopropyl-piperidin-3-yloxy)-benzamide,
N-(5-{4-[3-(1-Ethyl-propyl)-ureido]-2-methoxy-phenoxy}-thiazol-2-yl)-4-(1-isopropyl-piperidin-4-ylmethoxy)-benzamide,
N-(4-{2-Ethoxy-4-[3-(1-ethyl-propyl)-ureido]-phenoxy}-phenyl)-4-(8-methyl-8-aza-bicyclo[3.2.1]oct-(3-endo)-yloxy)-benzamide,
N-(4-{4-[3-(1-Ethyl-propyl)-ureido]-2-methoxy-phenoxy}-phenyl)-4-(8-methyl-8-aza-bicyclo[3.2.1]oct-(3-endo)-yloxy)-benzamide,
N-(4-{2-Ethoxy-4-[3-(1-ethyl-propyl)-ureido]-phenoxy}-phenyl)-N-ethyl-4-(8-methyl-8-aza-bicyclo[3.2.1]oct-(3-endo)-yloxy)-benzamide,
N-Ethyl-N-(4-{4-[3-(1-ethyl-propyl)-ureido]-2-methoxy-phenoxy}-phenyl)-4-(8-methyl-8-aza-bicyclo[3.2.1]oct-(3-endo)-yloxy)-benzamide,
N-(4-{4-[3-(1-Ethyl-propyl)-ureido]-2-methoxy-phenoxy}-phenyl)-3-methoxy-4-(8-methyl-8-aza-bicyclo[3.2.1]oct-(3-endo)-yloxy)-benzamide,
3-Chloro-N-(4-{4-[3-(1-ethyl-propyl)-ureido]-2-methoxy-phenoxy}-phenyl)-4-(8-methyl-8-aza-bicyclo[3.2.1]oct-(3-endo)-yloxy)-benzamide,
N-(4-{4-[3-(1-Ethyl-propyl)-ureido]-2-methoxy-phenoxy}-phenyl-(8-methyl-8-aza-bicyclo[3.2.1]oct-6-yloxy)-benzamide,
N-(4-{4-[3-(1-Ethyl-propyl)-ureido]-2-methyl-phenoxy}-phenyl)-4-(8-methyl-8-aza-bicyclo[3.2.1]oct-(3-endo)-yloxy)-benzamide,
N-(4-{4-[3-(1-Ethyl-propyl)-ureido]-phenoxy}-2-fluoro-phenyl)-4-(8-methyl-8-aza-bicyclo[3.2.1]oct-(3-endo)-yloxy)-benzamide,
N-(4-{4-[3-(1-Ethyl-propyl)-ureido]-2-methoxy-phenoxy}-phenyl)-2-fluoro-4-(8-methyl-8-aza-bicyclo[3.2.1]oct-(3-endo)-yloxy)-benzamide,
N-(4-{2-Chloro-4-[3-(1-ethyl-propyl)-ureido]-phenoxy}-phenyl)-N-ethyl-4-(8-methyl-8-aza-bicyclo[3.2.1]oct-(3-endo)-yloxy)-benzamide,
N-(4-{4-[3-(1-Ethyl-propyl)-ureido]-2-methoxy-phenoxy}-phenyl)-4-(2,2,6,6-Tetramethyl-piperidin-4-yloxy)-benzamide,
N-(4-{4-[3-Ethyl-propyl)-ureido]-2-methoxy-phenoxy}-phenyl)-4-(1,2,2,6,6-pentamethyl-piperidin-4-yloxy)-benzamide,
N-(4-{4-[3-(1-Ethyl-propyl)-ureido]-2-methoxy-phenoxy}-phenyl)-4-(2-methyl-2-aza-bicyclo[2.2.2]oct-(5-cis)-yloxy)-benzamide,
N-(5-{4-[3-(1-Ethyl-propyl)-ureido]-2-methoxymethyl-phenoxy}-thiazol-2-yl-4-(1-isobutyl-1,2,3,6-Tetrahydro-pyridin-4-yl)-benzamide,
N-(5-{4-[3-(1-Ethyl-propyl)-ureido]-2-methoxymethyl-phenoxy}-thiazol-2-yl)-4-(1-propyl-1,2,3,6-Tetrahydro-pyridin-4-yl)-benzamide,
4-(1-Butyl-1,2,3,6-Tetrahydro-pyridin-4-yl)-N-(5-{4-[3-(1-ethyl-propyl)-ureido]-2-methoxymethyl-phenoxy}-thiazol-2-yl)-benzamide,
N-(5-{4-[3-(1-Ethyl-propyl)-ureido]-2-methoxymethyl-phenoxy}-thiazol-2-yl)-4-[1-(3-methyl-butyl)-1,2,3,6-Tetrahydro-pyridin-4-yl]-benzamide,
N-(5-{4-[3-(1-Ethyl-propyl)-ureido]-2-methoxymethyl-phenoxy}-thiazol-2-yl-4-(1-isopropyl-piperidin-4-yl)-benzamide,
3-(4-Hydroxy-piperidin-1-ylmethyl)-1-isopropyl-1H-indole-6-carboxylic acid (5-{4-[3-(1-ethyl-propyl)-ureido]-2-methoxymethyl-phenoxy}-thiazol-2-yl)-amide,
2-[2-(4-Hydroxy-piperidin-1-yl)-ethyl]-benzofuran-6-carboxylic acid (5-{4-[3-(1-ethyl-propyl)-ureido]-2-methoxymethyl-phenoxy}-thiazol-2-yl)-amide,
N-(5-{4-[3-(1-Ethyl-propyl)-ureido]-2-methoxy-phenoxy}-thiazol-2-yl)-4-(3-piperidin-1-yl-propoxy)-benzamide,
N-(4-{4-[3-(1-Ethyl-propyl)-ureido]-2-methoxy-phenoxy}-phenyl)-4-(1-isopropyl-piperidin-4-yloxy)-benzamide,
4-(1-Butyl-piperidin-4-yloxy)-N-(4-{4-[3-(1-ethyl-propyl)-ureido]-2-methyl-phenoxy}-phenyl)-benzamide,
N-(4-{4-[3-(1-Ethyl-propyl)-ureido]-phenoxy}-3-methoxymethyl-phenyl-4-(1-isopropyl-piperidin-4-yloxy)-benzamide,
N-(4-{4-[3-(1-Ethyl-propyl)-ureido]-phenoxy}-3-methoxy-phenyl)-4-(1-isopropyl-piperidin-4-yloxy)-benzamide,
N-(5-{4-[3-(1-Ethyl-propyl)-ureido]-2-methylcarbamoylmethyl-phenoxy}-thiazol-2-yl)-4-(1-isobutyl-1,2,3,6-Tetrahydro-pyridin-4-yl)-benzamide,
N-(4-{5-[3-(1-Ethyl-propyl)-ureido]-2-methoxy-phenoxy}-phenyl)-4-(1-isopropyl-piperidin-4-yloxy)-benzamide,
{(4-cis-[4-(4-{4-[3-(1-Ethyl-propyl)-ureido]-2-methoxy-phenoxy}-phenylcarbamoyl)-phenoxy]-cyclohexyl}-trimethyl-ammonium,
N-(4-{4-[3-(1-Ethyl-propyl)-ureido]-phenoxy}-3-methyl-phenyl)-4-(3-piperidin-1-yl-propoxy)-benzamide,
N-(5-{4-[3-(1-Ethyl-propyl)-ureido]-2-methoxymethyl-phenoxy}-thiazol-2-yl)-4-(1-isopropyl-piperidin-4-ylmethyl)-benzamide,
N-(5-{4-[3-(1-Ethyl-propyl)-ureido]-2-methoxymethyl-phenoxy}-thiazol-2-yl)-4-(1-isopropyl-piperidin-4 ylidenemethyl)-benzamide,
4-[1-(2-Dimethylamino-Acetyl)-1,2,3,6-Tetrahydro-pyridin-4-yl]-N-(5-{4-[3-(1-ethyl-propyl)-ureido]-2-methoxymethyl-phenoxy}-thiazol-2-yl)-benzamide,
1-Isopropyl-2-(2-piperidin-1-yl-ethyl)-1H-benzoimidazole-5-carboxylic acid (5-{4-[3-(1-ethyl-propyl)-ureido]-2-methoxy-phenoxy}-thiazol-2-yl)-amide,
1-Isopropyl-2-(2-piperidin-1-yl-ethyl)-1H-benzoimidazole-5-carboxylic acid (4-{4-[3-(1-ethyl-propyl)-ureido]-phenoxy}-3-methyl-phenyl)-amide,
1-[3-(4-Hydroxy-piperidin-1-yl)-propyl]-1H-indole-5-carboxylic acid (5-{4-[3-(1-ethyl-propyl)-ureido]-2-methoxy-phenoxy}-thiazol-2-yl)-amide,
1-(2-Piperidin-1-yl-ethyl)-1H-indole-5-carboxylic acid (5-{4-[3-(1-ethyl-propyl)-ureido]-2-methoxy-phenoxy}-thiazol-2-yl)amide,
4-[1,4′]Bipiperidin-1′-yl-N-(5-{4-[3-(1-ethyl-propyl)-ureido]-2-methoxy-phenoxy}-thiazol-2-yl)-benzamide,
4-[Ethyl-(3-piperidin-1-yl-propionyl)-amino]-N-(5-{4-[3-(1-ethyl-propyl)-ureido]-2-methoxy-phenoxy}-thiazol-2-yl)-benzamide,
4-[Acetyl-(2-piperidin-1-yl-ethyl)-amino]-N-(5-{4-[3-(1-ethyl-propyl)-ureido]-2-methoxy-phenoxy}-thiazol-2-yl)-benzamide,
4-[Ethyl-(3-piperidin-1-yl-propyl)-amino]-N-(5-{4-[3-(1-ethyl-propyl)-ureido]-2-methoxy-phenoxy}-thiazol-2-yl)-benzamide,
N-(5-{4-[3-(1-Ethyl-propyl)-ureido]-2-methoxy-phenoxy}-thiazol-2-yl)-4-(3-piperidin-1-yl-propionylamino)-benzamide,
4-[Ethyl-(3-piperidin-1-yl-propionyl)-amino]-N-(5-{4-[3-(1-ethyl-propyl)-ureido]-phenoxy}-thiazol-2-yl)-benzamide,
4-[Acetyl-(2-piperidin-1-yl-ethyl)-amino]-N-(4-{4-[3-(1-ethyl-propyl)-ureido]-phenoxy}-3-methyl-phenyl)-benzamide,
4-(4-Ethyl-piperazine-1-carbonyl)-N-(4-{4-[3-(1-ethyl-propyl)-ureido]-phenoxy}-3-methyl-phenyl)-benzamide,
5-(3-Isopropyl-ureido)-2-(4-{[1-(2-piperidin-1-yl-ethyl)-1H-indole-5-carbonyl]-amino}-phenoxy)-methyl benzoate,
4-(1-Butyl-piperidin-4-yloxy)-N-(4-{2-ethoxy-4-[3-(1-ethyl-propyl)-ureido]-phenoxy}-phenyl-3-methoxy-benzamide,
4-(1-Butyl-piperidin-4-yloxy)-N-(4-{4-[3-(1-ethyl-propyl)-ureido]-phenoxy}-2-fluoro-phenyl)-3-methyl-benzamide,
N-(4-{4-[3-(1-Ethyl-propyl)-ureido]-2-methoxymethyl-phenoxy}-3-methyl-phenyl)-4-(1-isopropyl-piperidin-4-yloxy)-benzamide,
4-(1-Butyl-piperidin-4-yloxy)-N-ethyl-N-(4-{4-[3-(1-ethyl-propyl)-ureido]-2-methoxy-phenoxy}-phenyl)-benzamide,
N-(4-{4-[3-(1-Ethyl-propyl)-ureido]-2-methoxy-phenoxy}-phenyl)-4-(1-methyl-piperidin-4-yloxy)-benzamide,
4-(1-Butyl-piperidin-4-yloxy)-N-ethyl-N-(4-{4-[3-(1-ethyl-propyl)-ureido]-2-methoxy-phenoxy}-phenyl)-3-methoxy-benzamide,
4-(1-Butyl-piperidin-4-yloxy)-N-ethyl-N-(4-{4-[3-(1-ethyl-propyl)-ureido]-2-methoxy-phenoxy}-phenyl)-3-methyl-benzamide,
N-(5-{4-[3-(1-Ethyl-propyl)-ureido]-2-methoxymethyl-phenoxy}-thiazol-2-yl)-4-(1-isopropyl-1,2,3,6-Tetrahydro-pyridin-4-yl)-benzamide,
1-(3-Piperidin-1-yl-propyl)-1H-indole-5-carboxylic acid (4-{4-[3-(1-ethyl-propyl)-ureido]-2-methoxy-phenoxy}-3-methyl-phenyl)-amide,
1-(2-Piperidin-1-yl-ethyl)-1H-indole-5-carboxylic acid (4-{4-[3-(1-ethyl-propyl)-ureido]-phenoxy}-3-methyl-phenyl)-amide,
4-(1-Butyl-piperidin-4-yloxy)-N-(4-{4-[3-(1-ethyl-propyl)-ureido]-2-methoxy-phenoxy}-phenyl)-N-2,2,2-trifluoro-ethyl)-benzamide,
N-(4-{4-[3-(1-Ethyl-propyl)-ureido]-2-methoxy-phenoxy}-phenyl)-4-(8-methyl-8-aza-bicyclo[3.2.1]oct-(3-endo-yloxy)-N-(2,2,2-trifluoro-ethyl)-benzamide,
4-(1-Butyl-piperidin-4-yloxy)-N-(4-{4-[3-(1-ethyl-propyl)-ureido]-2-methoxy-phenoxy}-phenyl)-3,5-dimethyl-benzamide,
N-(4-{4-[3-(1-Ethyl-propyl)-ureido]-2-methoxy-phenoxy}-phenyl)-4-[1,(cis,cis-2,6)-trimethyl-piperidin-(cis-4)-yloxy]-benzamide,
2-Chloro-N-(4-{4-[3-(1-ethyl-propyl)-ureido]-2-methoxy-phenoxy}-phenyl)-4-(8-methyl-8-aza-bicyclo[3.2.1]oct-(3-endo)-yloxy)-benzamide,
N-(4-{4-[3-(1-Ethyl-propyl)-ureido]-2-methoxy-phenoxy}-phenyl)-4-[1,(cis,cis-2,6)-trimethyl-piperidin-(trans-4)-yloxy]-benzamide,
4-(1-Butyl-piperidin-4-yloxy)-3-chloro-N-ethyl-N-(4-{4-[3-(1-ethyl-propyl)-ureido]-2-methoxy-phenoxy}-phenyl)-benzamide,
4-(1-Butyl-piperidin-4-yloxy)-N-(4-{4-[3-(1-ethyl-propyl)-ureido]-2-methoxy-phenoxy}-phenyl)-benzamide,
4-(1-Butyl-piperidin-4-yloxy)-N-(4-{4-[3-(1-ethyl-propyl)-ureido]-2-propoxy-phenoxy}-phenyl)-3-methyl-benzamide,
4-(1-Butyl-piperidin-4-yloxy)-N-(4-{4-[3-(1-ethyl-propyl)-ureido]-2-methoxy-phenoxy}phenyl)-2-fluoro-benzamide,
4-(1-Butyl-piperidin-4-yloxy)-N-(4-{4-[3-(1-ethyl-propyl)-ureido]-phenoxy}phenyl)-benzamide,
1-(4-{1-[4-(1-Butyl-piperidin-4-yloxy)-benzoyl]-2,3-dihydro-1H-indol-5-yloxy}3-methoxy-phenyl-3-(1-ethyl-propyl)-urea,
4-(1-Butyl-piperidin-4-yloxy)-N-(4-{4-[3-(1-ethyl-propyl)-ureido]-2-methoxymethyl-phenoxy}-3-methyl-phenyl)-benzamide,
N-(4-{4-[3-(1-Ethyl-propyl)-ureido]-phenoxy}-phenyl)-4-(1-isopropyl-piperidin-4-yloxy)-benzamide,
4-(1-Butyl-piperidin-4-yloxy)-N-(4-{4-[3-(1-ethyl-propyl)-ureido]-2-fluoro-phenoxy}-phenyl)-benzamide,
1-(1-Ethyl-propyl)-3-(3-methoxy-4-{1-[4-(8-methyl-8-aza-bicyclo[3.2.1]oct-(3-endo)-yloxy)-benzoyl]-2,3-dihydro-1H-indol-5-yloxy}-phenyl)-urea,
N-(4-{4-[3-Ethyl-propyl)-ureido]-2-methoxy-phenoxy}-phenyl)-4-(8-methyl-8-aza-bicyclo[3.2.1]oct-(3-exo)-yloxy)-benzamide,
N-(4-{2-Ethyl-4-[3-(1-ethyl-propyl)-ureido]-phenoxy}-phenyl)-4-(8-methyl-8-aza-bicyclo[3.2.1]oct-(3-endo)-yloxy)-benzamide,
4-(1-Butyl-piperidin-4-yloxy)-N-{4-[4-(3-isopropyl-ureido)-phenoxy]-2-methoxy-phenyl}benzamide,
1-(4-{1-[4-(1-Butyl-piperidin-4-yloxy)-3-methyl-benzoyl]-2,3-dihydro-1H-indol-5-yloxy}-3-methoxy-phenyl)-3-(1-ethyl-propyl)-urea,
4-(1-Butyl-piperidin-4-yloxy)-N-(4-{2-ethyl-4-[3-(1-ethyl-propyl)-ureido]-phenoxy}-phenyl)-benzamide,
N-(4-{4-[3-(1-Ethyl-propyl)-ureido]-2-methoxy-phenoxy}-phenyl)-4-(8-isopropyl-piperidin-4-ylmethoxy)-benzamide,
N-(4-{4-[3-(1-Ethyl-propyl)-ureido]-2-trifluoromethyl-phenoxy}-phenyl)-4-(8-methyl-8-aza-bicyclo[3.2.1]oct-(3-endo)-yloxy)-benzamide,
4-(1-Butyl-piperidin-4-yloxy)-N-{4-[4-(3-dimethylamino-ureido)-phenoxy]-3-methoxymethyl-phenyl}-3-methyl-benzamide,
4-(1-Butyl-piperidin-4-yloxy)-N-(4-{4-[3-N,N-dimethyl-amino)-ureido]-phenoxy}-3-methoxymethyl-phenyl)-3-methoxy-benzamide,
4-(1-Butyl-piperidin-4-yloxy)-N-(4-{4-[3-(1-ethyl-propyl)-ureido]-phenoxy}-3-trifluoromethyl-phenyl)-benzamide,
4-(1-Butyl-piperidin-4-yloxy)-N-{4-[4-(3-isopropyl-ureido)-benzyl]-phenyl}-benzamide,
N-(4-{4-[3-(1-Ethyl-propyl)-ureido]-phenoxy}-phenyl)-4-(8-methyl-8-aza-bicyclo[3.2.1]oct-(3-endo)-yloxy)-benzamide,
N-(4-{2-Chloro-4-[3-(1-ethyl-propyl)-ureido]-phenoxy}-phenyl)-4-(8-methyl-8-aza-bicyclo[3.2.1]oct-(3-endo)-yloxy)-benzamide,
N-(4-{4-[3-(1-Ethyl-propyl)-ureido]-2-methoxymethyl-phenoxy}-phenyl)-4-(8-methyl-8-aza-bicyclo[3.2.1]oct-(3-endo)-yloxy)-benzamide,
N-(4-{4-[3-(1-Ethyl-propyl)-ureido]-2-fluoro-phenoxy}-phenyl)-4-(8-methyl-8-aza-bicyclo[3.2.1]oct-(3-endo)-yloxy)-benzamide,
4-(1-Butyl-piperidin-4-yloxy)-N-(4-{2-chloro-4-[3-(1-ethyl-propyl)-ureido]-phenoxy}-2-fluoro-phenyl)-benzamide,
4-(1-Butyl-piperidin-4-yloxy)-N-(4-{2-ethoxy-4-[3-(1-ethyl-propyl)-ureido]-phenoxy}-phenyl)-3-methyl-benzamide,
N-(4-{4-[3-(1-Ethyl-propyl)-ureido]-3-fluoro-phenoxy}-phenyl)-4-(8-methyl-8-aza-bicyclo[3.2.1]oct-(3-endo)-yloxy)-benzamide,
4-(1-Butyl-piperidin-4-yloxy)-N-(4-{4-[3-(1-ethyl-propyl)-ureido]-2-methoxy-phenoxy}phenyl)-3-fluoro-benzamide,
4-(1-Butyl-piperidin-4-yloxy)-N-(4-{4-[3-(1-ethyl-propyl)-ureido]-3-fluoro-phenoxy}-phenyl)-benzamide,
4-(1-Butyl-piperidin-4-yloxy)-N-{4-[4-(3-isopropyl-ureido)-2-methoxy-phenoxy]-phenyl}-benzamide,
4-(1-Butyl-piperidin-4-yloxy)-N-(4-{4-[3-(1-ethyl-propyl)-ureido]-phenoxy}-2-fluoro-phenyl)-benzamide,
4-(1-Butyl-piperidin-4-yloxy)-N-(4-{4-[3-(1-ethyl-propyl)-ureido]-2-methoxy-phenoxy}phenyl)-3-trifluoromethyl-benzamide,
4-(1-Butyl-piperidin-4-yloxy)-N-(4-{4-[3-(1-ethyl-propyl)-ureido]-phenoxy}-2-methoxy-phenyl)-3-methyl-benzamide,
4-(1-Butyl-piperidin-4-yloxy)-N-(4-{4-[3-(1-ethyl-propyl)-ureido]-phenoxy}phenyl)-3-methyl-benzamide,
N-(4-{4-[3-(1-Ethyl-propyl)-ureido]-phenoxy}-3-methyl-phenyl)-4-(8-methyl-8-aza-bicyclo[3.2.1]oct-(3-endo)-yloxy)-benzamide,
4-(1-Butyl-piperidin-4-yloxy)-N-{4-[4-(3-isopropyl-ureido)-phenoxy]-3,5-dimethyl-phenyl}-benzamide,
N-(4-{3-[3-(1-Ethyl-propyl)-ureido]-phenoxy}-3-methyl-phenyl)-4-(1-isopropyl-piperidin-4-yloxy)-benzamide,
4-(1-Butyl-piperidin-4-yloxy)-N-{4-[4-(3-isopropyl-ureido)-phenoxy]-2,5-dimethyl-phenyl}benzamide,
4-(1-Butyl-piperidin-4-yloxy)-N-{4-[4-(3-isopropyl-ureido)-3-methoxy-phenoxy]-phenyl}-benzamide,
4-(1-Isopropyl-1,2,3,6-Tetrahydro-pyridin-4-yl)-N-{4-[4-(3-isopropyl-ureido)-2-methoxy-phenoxy]-phenyl}-benzamide,
(1-Ethyl-propyl)-carbamate of 4-{4-[4-(1-isopropyl-piperidin-4-yloxy)-benzoylamino]-2-methyl-phenoxy}-phenyl,
N-(4-{4-[3-Ethyl-propyl)-ureido]-2-methoxy-phenoxy}-phenyl)-4-(1-methyl-1,2,3,6-Tetrahydro-pyridin-4-yl)-benzamide,
N-(4-{4-[3-(1-Ethyl-propyl)-ureido]-2-methoxy-phenoxy}-3-methyl-phenyl)-4-(1-ethyl-1,2,3,6-Tetrahydro-pyridin-4-yl)-benzamide,
4-(1-Butyl-1,2,3,6-Tetrahydro-pyridin-4-yl)-N-(4-{4-[3-(1-ethyl-propyl)-ureido]-2-methoxy-phenoxy}-3-methyl-phenyl)-benzamide,
4-(1-Isobutyl-1,2,3,6-Tetrahydro-pyridin-4-yl)-N-{4-[4-(3-isopropyl-ureido)-2-methoxy-phenoxy]-phenyl}-benzamide,
1-(2-Piperidin-1-yl-ethyl)-1H-indole-5-carboxylic acid (4-{4-[3-(1-ethyl-propyl)-ureido]-2-methylcarbamoyl-phenoxy}-phenyl)-amide,
4-[Acetyl-(3-piperidin-1-yl-propyl)-amino]-N-(4-{4-[3-(1-ethyl-propyl)-ureido]-phenoxy}-3-methyl-phenyl)-benzamide,
N-Ethyl-N-(4-{4-[3-(1-ethyl-propyl)-ureido]-2-methoxy-phenoxy}-phenyl)-3-fluoro-4-(8-methyl-8-aza-bicyclo[3.2.1]oct-(3-endo)-yloxy)-benzamide,
N-(4-{4-[3-(1-Ethyl-propyl)-ureido]-2-methoxy-phenoxy}-phenyl)-3-fluoro-4-(8-methyl-8-aza-bicyclo[3.2.1]oct-(3-endo)-yloxy)-benzamide,
N-(4-{4-[3-(1-Ethyl-propyl)-ureido]-2-methoxy-phenoxy}3-methyl-phenyl-4-(1-methyl-1,2,3,6-Tetrahydro-pyridin-4-yl)-benzamide,
4-(1-Butyl-piperidin-4-yloxy)-N-{4-[4-(3-isopropyl-ureido)-phenoxy]-phenyl}-benzamide,
4-[1-(2-Dimethylamino-Acetyl)-piperidin-4-yloxy]-N-(4-{4-[3-(1-ethyl-propyl)-ureido]-2-methoxy-phenoxy}-phenyl)-benzamide,
4-(1-Butyl-piperidin-4-yloxy-N-{4-[2-dimethylamino-Acetylamino)-2-methoxy-phenoxy]-phenyl}-benzamide,
4-(1-Butyl-piperidin-4-yloxy)-N-{4-[3-hydroxymethyl-4-(3-isopropyl-ureido)-phenoxy]-phenyl}-benzamide,
5-[3-(1-ethyl-propyl)-ureido]-N-methyl-2-{4-[4-(3-piperidin-1-yl-propoxy)-benzoylamino]-phenoxy}-benzamide,
4-[(4-cis)-Dimethylamino-cyclohexyloxy]-N-(4-{4-[3-(1-ethyl-propyl)-ureido]-2-methoxy-phenoxy}-phenyl)-benzamide,
5-[3-(1-Ethyl-propyl)-ureido]-2-(4-{4-[3-(4-hydroxy-piperidin-1-yl)-propoxy]-benzoylamino}-phenoxy)-N-methyl-benzamide,
4-(1-Butyl-piperidin-4-yloxy)-N-{4-[4-(3-isopropyl-ureido)-phenylsulfanyl]-phenyl}-benzamide,
N-(4-{4-[3-(1-Ethyl-propyl)-ureido]-3-fluoro-phenoxy}-phenyl)-4-(1-methyl-1,2,3,6-Tetrahydro-pyridin-4-yl)-benzamide,
N-(4-{4-[3-(1-Ethyl-propyl)-ureido]-2-methoxymethyl-phenoxy}-phenyl)-4-(1-isopropyl-1,2,3,6-Tetrahydro-pyridin-4-yl)-benzamide,
N-(4-{4-[3-Ethyl-propyl)-ureido]-2-methoxy-phenoxy}-3-methyl-phenyl)-4-(1-isopropyl-1,2,3,6-Tetrahydro-pyridin-4-yl)-benzamide,
N-(4-{4-[3-Ethyl-propyl)-ureido]-2-methoxy-phenoxy}-phenyl)-4-(1-isopropyl-1,2,3,6-Tetrahydro-pyridin-4-yl)-benzamide,
N-(4-{4-[3-(1-Ethyl-propyl)-ureido]-2-methoxy-phenoxy}-3-methyl-phenyl)-4-(1-propyl-1,2,3,6-Tetrahydro-pyridin-4-yl)-benzamide,
1-[3-(4-Hydroxy-piperidin-1-yl)propyl]-1H-indole-5-carboxylic acid (4-{4-[3-(1-ethyl-propyl)-ureido]-2-methylcarbamoyl-phenoxy}-phenyl)-amide,
1-(3-Piperidin-1-yl-propyl)-1H-indole-5-carboxylic acid (4-{4-[3-(1-ethyl-propyl)-ureido]-phenoxy}-3-methyl-phenyl)-amide,
3-Methyl-1-(2-piperidin-1-yl-ethyl)-1H-indole-5-carboxylic acid (4-{4-[3-(1-ethyl-propyl)-ureido]-phenoxy}-3-methyl-phenyl)-amide,
3-Acetyl-1-(2-piperidin-1-yl-ethyl)-1H-indole-5-carboxylic acid (4-{4-[3-(1-ethyl-propyl)-ureido]-phenoxy}-3-methyl-phenyl)-amide,
4-[Acetyl(3-piperidin-1-yl-propyl)-amino]-N-(5-{4-[3-(1-ethyl-propyl)-ureido]-2-methoxy-phenoxy}-thiazol-2-yl)-benzamide,
2-(4-{4-[Acetyl-(3-diethylamino-propyl)-amino]-benzoylamino}-phenoxy)-5-[3-(1-ethyl-propyl)-ureido]-N-methyl-benzamide,
4-[Ethyl-(3-piperidin-1-yl-propionyl)-amino]-N-(4-{4-[3-(1-ethyl-propyl)-ureido]-phenoxy}-3-methyl-phenyl)-benzamide,
4-[Ethyl-(3-piperidin-1-yl-propyl)-amino]-N-(4-{4-[3-(1-ethyl-propyl)-ureido]-phenoxy}-3-methyl-phenyl)-benzamide,
N-(4-{4-[3-(1-Ethyl-propyl)-ureido]-phenoxy}-3-methyl-phenyl)-4-(3-piperidin-1-yl-propionylamino)-benzamide,
1-(3-Piperidin-1-yl-propyl)-1H-indole-5-carboxylic acid (4-{4-[3-(1-ethyl-propyl)-ureido]-2-methylcarbamoyl-phenoxy}-phenyl)-amide,
4-(1-Benzyl-piperidin-4-yloxy)-N-(4-{4-[3-(1-ethyl-propyl)-ureido]-2-methoxy-phenoxy}-phenyl)-benzamide,
N-(4-{4-[3-(1-Ethyl-propyl)-ureido]-2-methoxy-phenoxy}-phenyl)-4-(piperidin-4-yloxy)-benzamide,
N-(4-{4-[3-(1-Ethyl-propyl)-ureido]-phenoxy}-3-methyl-phenyl)-piperidin-4-yloxy)-benzamide,
N-(4-{4-[3-(1-Ethyl-propyl)-ureido]-2-methoxy-phenoxy}-phenyl)-4-(1-propyl-piperidin-4-yloxy)-benzamide,
4-{4-[4-(4-{4-[3-(1-ethyl-propyl)-ureido]-2-methoxy-phenoxy}phenylcarbamoyl)-phenoxy]-piperidin-1-yl}butyl acetate,
4-[1-(3-Dimethylamino-propyl)-piperidin-4-yloxy]-N-(4-{4-[3-(1-ethyl-propyl)-ureido]-2-methoxy-phenoxy}-phenyl)-N-(2-methoxy-ethyl)-benzamide,
4-[1-(3-Dimethylamino-propyl)-piperidin-4-yloxy]-N-(4-{4-[3-(1-ethyl-propyl)-ureido]-2-methoxy-phenoxy}-phenyl)-N-isobutyl-benzamide,
4-(1-Butyl-piperidin-4-yloxy)-N-(4-{4-[3-(1-ethyl-propyl)-ureido]-2-methoxy-phenoxy}-3-methyl-phenyl)-benzamide,
4-(8-Butyl-8-aza-bicyclo[3.2.1]oct-(3-endo-yloxy)-N-(4-{4-[3-(1-ethyl-propyl)-ureido]-2-methoxy-phenoxy}-phenyl)-benzamide,
4-(8-Ethyl-8-aza-bicyclo[3.2.1]oct-(3-endo-yloxy)-N-(4-{4-[3-(1-ethyl-propyl)-ureido]-2-methoxy-phenoxy}-phenyl)-benzamide,
N-(4-{4-[3-(1-Ethyl-propyl)-ureido]-2-methoxy-phenoxy}-phenyl)-4-[8-(3-methoxy-propyl)-8-aza-bicyclo[3.2.1]oct-(3-endo)-yloxy]-benzamide,
N-(4-{4-[3-(1-Ethyl-propyl)-ureido]-2-methoxy-phenoxy}-phenyl)-4-[8-(2-methoxy-ethyl)-8-aza-bicyclo[3.2.1]oct-(3-endo)-yloxy]-benzamide,
N-(4-{4-[3-Ethyl-propyl)-ureido]-2-methoxy-phenoxy}-phenyl)-4-[1-(4,4,4-trifluoro-butyl)-piperidin-4-yloxy]-benzamide,
4-[1-(2-Diethylamino-ethyl)-piperidin-4-yloxy]-N-(4-{4-[3-(1-ethyl-propyl)-ureido]-2-methoxy-phenoxy}-3-methyl-phenyl)-benzamide,
N-(4-{4-[3-(1-Ethyl-propyl)-ureido]-2-methoxy-phenoxy}-phenyl)-4-[1-(4-fluoro-butyl)-piperidin-4-yloxy]-benzamide,
4-[1-(1-Ethyl-propyl)-piperidin-4-yloxy]-N-(4-{4-[3-(1-ethyl-propyl)-ureido]-phenoxy}-3-methyl-phenyl)-benzamide,
{4-[4-(4-{4-[3-(1-Ethyl-propyl)-ureido]-phenoxy}-3-methyl-phenylcarbamoyl)-phenoxy]-piperidin-1-yl}-ethyl acetate,
{4-[4-(4-{4-[3-(1-Ethyl-propyl)-ureido]-phenoxy}-3-methyl-phenylcarbamoyl)-phenoxy]-piperidin-1-yl}-acetic acid,
N-(4-{4-[3-(1-Ethyl-propyl)-ureido]-2-methoxy-phenoxy}-phenyl)-4-[1-(4-hydroxy-butyl)-piperidin-4-yloxy]-benzamide,
4-{(3-endo)-[4-(4-{4-[3-(1-ethyl-propyl)-ureido]-2-methoxy-phenoxy}phenylcarbamoyl)-phenoxy]-8-aza-bicyclo[3.2.1]oct-8-yl}-butyl acetate,
4-[8-(3-Dimethylamino-propyl)-8-aza-bicyclo[3.2.1]oct-(3-endo)-yloxy]-N-(4-{4-[3-(1-ethyl-propyl)-ureido]-2-methoxy-phenoxy}-phenyl)-benzamide,
4-[1-(3-Dimethylamino-propyl)-piperidin-4-yloxy]-N-(4-{4-[3-(1-ethyl-propyl)-ureido]-2-methoxy-phenoxy}-phenyl)-benzamide,
N-(4-{4-[3-(1-Ethyl-propyl)-ureido]-2-methoxy-phenoxy}-phenyl-4-(8-propyl-8-aza-bicyclo[3.2.1]oct-3-yloxy)-benzamide,
4-(1-sec-Butyl-piperidin-4-yloxy)-N-(4-{4-[3-(1-ethyl-propyl)-ureido]-2-methoxy-phenoxy}-phenyl)-benzamide,
4-(1-Butyl-piperidin-4-yloxy)-N-(4-{4-[3-(1-ethyl-propyl)-ureido]-2-methoxy-phenoxy}phenyl)-N-(2-methoxy-ethyl)-benzamide,
N-(4-{4-[3-(1-Ethyl-propyl)-ureido]-2-methoxy-phenoxy}-phenyl)-4-[8-(2-hydroxy-ethyl)-8-aza-bicyclo[3.2.1]oct-(3-endo)-yloxy]-benzamide,
N-(4-{4-[3-(1-Ethyl-propyl)-ureido]-2-methoxy-phenoxy}-phenyl)-4-[8-(4,4,4-trifluoro-butyl)-8-aza-bicyclo[3.2.1]oct-(3-endo)-yloxy]-benzamide,
N-(4-{4-[3-(1-Ethyl-propyl)-ureido]-2-methoxy-phenoxy}-phenyl)-4-[8-(3-hydroxy-propyl)-8-aza-bicyclo[3.2.1]oct-(3-endo)-yloxy]-benzamide,
N-(4-{4-[3-(1-Ethyl-propyl)-ureido]-2-methoxy-phenoxy}-phenyl)-4-(8-isopropyl-8-aza-bicyclo[3.2.1]oct-(3-endo)-yloxy)-benzamide,
4-(1-Cyclohexylmethyl-piperidin-4-yloxy)-N-(4-{4-[3-(1-ethyl-propyl)-ureido]-phenoxy}-3-methyl-phenyl)-benzamide,
4-[1-(2-Ethyl-butyl)-piperidin-4-yloxy]-N-(4-{4-[3-(1-ethyl-propyl)-ureido]-phenoxy}-3-methyl-phenyl)-benzamide,
N-(4-{4-[3-(1-Ethyl-propyl)-ureido]-phenoxy}-3-methyl-phenyl)-4-[1-(2-methoxy-ethyl)piperidin-4-yloxy]-benzamide,
N-(4-{4-[3-(1-Ethyl-propyl)-ureido]-phenoxy}-3-methyl-phenyl)-4-[1-(2-hydroxy-ethyl)-piperidin-4-yloxy]-benzamide,
N-(4-{4-[3-Ethyl-propyl)-ureido]-2-methoxy-phenoxy}-phenyl)-4-[8-(4-hydroxy-butyl)-8-aza-bicyclo[3.2.1]oct-(3-endo)-yloxy]-benzamide,
4-(8-Aza-bicyclo[3.2.1]oct-(3-endo)-yloxy)-N-(4-{4-[3-(1-ethyl-propyl)-ureido]-2-methoxy-phenoxy}-phenyl)-benzamide,
4-(8-Aza-bicyclo[3.2.1]oct-(3-endo)-yloxy)-N-(4-{4-[3-(1-ethyl-propyl)-ureido]-2-methoxy-phenoxy}-phenyl)-N-(2-methoxy-ethyl)-benzamide,
N-(4-{4-[3-(1-Ethyl-propyl)-ureido]-2-methoxy-phenoxy}-phenyl)-4-[1-(3-methoxy-propyl)-piperidin-4-yloxy]-benzamide,
N-(4-{4-[3-(1-Ethyl-propyl)-ureido]-2-methoxy-phenoxy}-phenyl)-4-[1-(2-hydroxy-ethyl)-piperidin-4-yloxy]-benzamide,
N-(4-{4-[3-(1-Ethyl-propyl)-ureido]-2-methoxy-phenoxy}-3-methyl-phenyl)-4-[1-(3-hydroxy-propyl)-piperidin-4-yloxy]-benzamide,
4-[1-(2-Ethoxy-ethyl)-piperidin-4-yloxy]-N-(4-{4-[3-(1-ethyl-propyl)-ureido]-2-methoxy-phenoxy}-phenyl)-benzamide,
N-(4-{[3-(1-Ethyl-propyl)-ureido]-2-methoxy-phenoxy}-phenyl)-4-[1-(2-methoxy-ethyl)-piperidin-4-yloxy]-benzamide,
N-(4-{4-[3-(1-Ethyl-propyl)-ureido]-2-methoxy-phenoxy}-3-methyl-phenyl)-4-[1-(3-methyl-butyl)-piperidin-4-yloxy]-benzamide,
4-(1-Butyl-piperidin-4-yloxy)-N-(4-{4-[3-(1-ethyl-propyl)-ureido]-2-methoxy-phenoxy}-phenyl)-N-isobutyl-benzamide,
4-(1-sec-Butyl-piperidin-4-yloxy)-N-(4-{4-[3-(1-ethyl-propyl)-ureido]-phenoxy}-3-methyl-phenyl)-benzamide,
N-(4-{4-[3-(1-Ethyl-propyl)-ureido]-2-methoxy-phenoxy}-phenyl)-4-[1-(3,3,3-trifluoro-propyl)-piperidin-4-yloxy]-benzamide,
N-(4-{4-[3-(1-Ethyl-propyl)-ureido]-2-methoxy-phenoxy}-phenyl)-4-[1-(3-hydroxy-propyl)-piperidin-4-yloxy]-benzamide,
N-(4-{4-[3-(1-Ethyl-propyl)-ureido]-2-methoxy-phenoxy}-phenyl)-4-[1-(3-methyl-butyl)-piperidin-4-yloxy]-benzamide,
4-[1-(2-Dimethylamino-ethyl)-piperidin-4-yloxy]-N-(4-{4-[3-(1-ethyl-propyl)-ureido]-2-methoxy-phenoxy}-phenyl)-benzamide,
4-(1-Butyl-piperidin-4-yloxy)-N-(4-{4-[3-(1-ethyl-propyl)-ureido]-2-methoxymethyl-phenoxy}-phenyl)-benzamide,
N-(4-{4-[3-(1-Ethyl-propyl)-ureido]-2-methoxy-phenoxy}-phenyl)-4-[1-(1-methyl-butyl)-piperidin-4-yloxy]-benzamide,
N-(4-{4-[3-(1-ethyl-propyl)-ureido]-2-methoxy-phenoxy}-phenyl)-4-[1-(Tetrahydro-pyran-4-yl)-piperidin-4-yloxy]-benzamide,
N-(4-{4-[3-(1-Ethyl-propyl)-ureido]-phenoxy}-3-methyl-phenyl)-4-[1-(1-hydroxymethyl-propyl)-piperidin-4-yloxy]-benzamide,
4-(1-Cyclobutyl-piperidin-4-yloxy)-N-(4-{4-[3-(1-ethyl-propyl)-ureido]-phenoxy}-3-methyl-phenyl)-benzamide,
N-(4-{4-[3-(1-Ethyl-propyl)-ureido]-phenoxy}-3-methyl-phenyl)-4-[1-(1-methyl-butyl)-piperidin-4-yloxy]-benzamide,
4-(1-Cyclopentyl-piperidin-4-yloxy)-N-(4-{4-[3-(1-ethyl-propyl)-ureido]-phenoxy}-3-methyl-phenyl)-benzamide,
N-(4-{4-[3-(1-Ethyl-propyl)-ureido]-phenoxy}-3-methyl-phenyl)-4-(1-propyl-piperidin-4-yloxy)-benzamide,
4-(1-Butyl-piperidin-4-yloxy)-N-(4-{4-[3-(1-ethyl-propyl)-ureido]-phenoxy}-3-methyl-phenyl)-benzamide,
N-(4-{4-[3-(1-Ethyl-propyl)-ureido]-phenoxy}-3-methyl-phenyl)-4-(1-methyl-piperidin-4-yloxy)-benzamide,
N-(4-{4-[3-(1-Ethyl-propyl)-ureido]-phenoxy}-3-methyl-phenyl)-4-[1-(3-methyl-butyl)-piperidin-4-yloxy]-benzamide,
4-(1-Ethyl-piperidin-4-yloxy)-N-(4-{4-[3-(1-ethyl-propyl)-ureido]-phenoxy}-3-methyl-phenyl)-benzamide,
N-(4-{4-[3-(1-Ethyl-propyl)-ureido]-phenoxy}-3-methyl-phenyl)-4-(1-isobutyl-piperidin-4-yloxy)-benzamide,
4-(1-Cyclopropyl-piperidin-4-yloxy)-N-(4-{4-[3-(1-ethyl-propyl)-ureido]-phenoxy}-3-methyl-phenyl)-benzamide,
4-(1-Butyl-piperidin-4-yloxy)-N-(4-{4-[3-(1-ethyl-propyl)-ureido]-2-methoxy-phenoxy}-2-fluoro-phenyl)-benzamide,
N-(4-{4-[3-(1-Ethyl-propyl)-ureido]-2-methoxy-phenoxy}-phenyl)-4-(8-methyl-8-aza-bicyclo[3.2.1]oct-(3-endo)-yloxy)-N-propyl-benzamide,
N-(4-{4-[3-(1-Ethyl-propyl)-ureido]-2-methoxy-phenoxy}2-fluoro-phenyl)-4-(8-methyl-8-aza-bicyclo[3.2.1]oct-(3-endo)-yloxy)-benzamide,
4-(1-Butyl-piperidin-4-yloxy)-N-(4-{4-[3-(1-ethyl-propyl)-ureido]-2-methoxy-phenoxy}-2-fluoro-phenyl)-3-methyl-benzamide,
4-(1-Butyl-piperidin-4-yloxy)-N-{8-[3-(1-ethyl-propyl)-ureido]-10,11-dihydro-dibenzo[b,f]oxepin-2-yl}benzamide,
N-(4-{4-[3-Ethyl-propyl)-ureido]-2-methoxy-phenoxy}-phenyl)-N-methyl-4-(8-methyl-8-aza-bicyclo[3.2.1]oct-(3-endo)-yloxy)-benzamide,
N-[4-(1-Butyl-piperidin-4-yloxy)-phenyl]-4-{4-[3-(1-ethyl-propyl)-ureido]-2-methoxy-phenoxy}-benzamide,
1-(4-{2-[4-(1-Butyl-piperidin-4-yloxy)-phenyl]-1-methyl-1H-benzoimidazol-5-yloxy}-3-methoxy-phenyl)-3-(1-ethyl-propyl)-urea,
1-(4-{2-[4-(1-Butyl-piperidin-4-yloxy)-phenyl]-1-propyl-1H-benzoimidazol-5-yloxy}-3-methoxy-phenyl)-3-(1-ethyl-propyl)-urea,
1-(4-{2-[4-(1-Butyl-piperidin-4-yloxy)-phenyl]-1-ethyl-1H-indol-5-yloxy}-3-methoxy-phenyl)-3-(1-ethyl-propyl)-urea,
1-(4-{4-[6-(1-Butyl-piperidin-4-yloxy)-benzooxazol-2-yl]-phenoxy}-3-methoxy-phenyl)-3-(1-ethyl-propyl)-urea,
1-(4-{4-[6-(1-Butyl-piperidin-4-yloxy)-benzooxazol-2-yl]-phenoxy}-phenyl)-3-(1-ethyl-propyl)-urea,
1-(1-Ethyl-propyl)-3-(3-methoxy-4-{4-[5-(1-methyl-piperidin-4-yloxy)-benzofuran-2-yl]-phenoxy}-phenyl)-urea,
4-(1-Butyl-piperidin-4-yloxy)-N-(4-{2-chloro-4-[3-(1-ethyl-propyl)-ureido]-phenoxy}-2-fluoro-phenyl)-3-methyl-benzamide,
4-(1-Butyl-piperidin-4-yloxy)-N-(4-{4-[3-(1-ethyl-propyl)-ureido]-phenoxy}phenyl)-N-(2-methoxy-ethyl)-3-methyl-benzamide,
4-(1-Butyl-piperidin-4-yloxy)-N-(4-{4-[3-(1-ethyl-propyl)-ureido]-2-methoxy-phenoxy}phenyl)-N-(2-methoxy-ethyl)-3-methyl-benzamide,
4-(1-Butyl-piperidin-4-yloxy)-N-(4-{4-[3-(1-ethyl-propyl)-ureido]-phenoxy}-2-fluoro-phenyl)-3-methoxy-benzamide,
4-(1-Butyl-piperidin-4-yloxy)-N-(4-{4-[3-(1-ethyl-propyl)-ureido]-3-fluoro-phenoxy}-phenyl)-3-methoxy-benzamide,
N-(4-{2-Ethoxy-4-[3-(1-ethyl-propyl)-ureido]-phenoxy}-phenyl)-3-methyl-4-(1-methyl-piperidin-4-yloxy)-benzamide,
N-(4-{4-[3-(1-Ethyl-propyl)-ureido]-3-fluoro-phenoxy}-phenyl)-3-methyl-4-(1-methyl-piperidin-4-yloxy)-benzamide,
N-(4-{4-[3-(1-Ethyl-propyl)-ureido]-phenoxy}-2-fluoro-phenyl)-3-methyl-4-(1-methyl-piperidin-4-yloxy)-benzamide,
N-(4-{2-Ethoxy-4-[3-(1-ethyl-propyl)-ureido]-phenoxy}-2-fluoro-phenyl)-3-methyl-4-(1-methyl-piperidin-4-yloxy)-benzamide,
4-(1-Butyl-piperidin-4-yloxy)-N-(4-{2-Ethoxy-4-[3-(1-ethyl-propyl)-ureido]-phenoxy}-2-fluoro-phenyl)-benzamide,
4-(1-Butyl-piperidin-4-yloxy)-N-(4-{2-Ethoxy-4-[3-(1-ethyl-propyl)-ureido]-phenoxy}-phenyl)-N-(2-methoxy-ethyl)-benzamide,
4-(1-Butyl-piperidin-4-yloxy)-N-(2-Ethoxy-ethyl)-N-(4-{4-[3-(1-ethyl-propyl)-ureido]-2-methoxy-phenoxy}-phenyl)-benzamide,
4-(1-Butyl-piperidin-1 yloxy)-N-(4-{4-[3-(1-ethyl-propyl)-ureido]-5-fluoro-2-methoxy-phenoxy}-phenyl)-benzamide,
4-(1-Butyl-piperidin-4-yloxy)-N-(4-{4-[3-(1-ethyl-propyl)-ureido]-2,5-difluoro-phenoxy}-phenyl)-benzamide,
1-(1-Methyl-piperidin-4-yl)-1H-indole-5-carboxylic acid (4-{4-[3-(1-ethyl-propyl)-ureido]-2-methoxy-phenoxy}-phenyl)-amide,
1-(1-Methyl-piperidin-4-yl)-1H-indole-5-carboxylic acid (4-{4-[3-(1-ethyl-propyl)-ureido]-phenoxy}-3-methoxymethyl-phenyl)-amide,
1-(4-(1-Butyl-piperidin-4-yl)-1H-indole-5-carboxylic acid (4-{4-[3-(1-ethyl-propyl)-ureido]-5-fluoro-2-methoxy-phenoxy}-phenyl)-amide,
1-(1-Butyl-piperidin-4-yl)-1H-indole-5-carboxylic acid (4-{2-ethoxy-4-[3-(1-ethyl-propyl)-ureido]-phenoxy}-2-fluoro-phenyl)-amide,
1-(1-Butyl-piperidin-4-yl)-1H-indole-5-carboxylic acid (4-{2-ethoxy-4-[3-(1-ethyl-propyl)-ureido]-phenoxy}-phenyl)-amide,
4-(1-Butyl-piperidin-4-yloxy)-N-(4-{4-[3-(1-ethyl-propyl)-ureido]-5-fluoro-2-methoxy-phenoxy}-phenyl)-2,5-difluoro-benzamide,
4-(1-Butyl-piperidin-4-yloxy)-N-(4-{4-[3-(1-ethyl-propyl)-ureido]-2,5-difluoro-phenoxy}-phenyl)-2,5-difluoro-benzamide,
4-(1-Butyl-piperidin-4-yloxy)-N-(4-{2-Ethoxy-4-[3-(1-ethyl-propyl)-ureido]-phenoxy}-phenyl)-2,5-difluoro-benzamide,
4-(1-Butyl-3-fluoro-piperidin-4-yloxy)-N-(4-{2-Ethoxy-4-[3-(1-ethyl-propyl)-ureido]-phenoxy}-phenyl)-benzamide,
4-(1-Dimethylamino-piperidin-4-yloxy)-N-(4-{2-Ethoxy-4-[3-(1-ethyl-propyl)-ureido]-phenoxy}-phenyl)-benzamide,
4-(1-Butyl-pyrrolidin-3-yloxy)-N-(4-{2-Ethoxy-4-[3-(1-ethyl-propyl)-ureido]-phenoxy}-phenyl)-benzamide,
4-[(1-Butyl-piperidin-4-yl)-methyl-amino]-N-(4-{4-[3-(1-ethyl-propyl)-ureido]-2-methoxy-phenoxy}-phenyl)-benzamide,
4-[(1-Butyl-piperidin-4-yl)-methyl-amino]-N-(4-{4-[3-(1-ethyl-propyl)-ureido]-5-fluoro-2-methoxy-phenoxy}-phenyl)-benzamide,
4-[(1-Butyl-piperidin-4-yl)-methyl-amino]-N-(4-{4-[3-(1-ethyl-propyl)-ureido]-3-fluoro-phenoxy}-phenyl)-benzamide,
4-(1-Butyl-piperidin-4-yloxy)-2-chloro-N-(4-{4-[3-(1-ethyl-propyl)-ureido]-2-methoxy-phenoxy}-phenyl)-benzamide,
1-(1-Butyl-piperidin-4-yl)-2,3-dihydro-1H-indole-5-carboxylic acid (4-{2-Ethoxy-4-[3-(1-ethyl-propyl)-ureido]-phenoxy}-phenyl)-amide,
4-(1-Butyl-piperidin-4-yloxy)-N-(4-{4-[3-(1-ethyl-propyl)-ureido]-2-methoxy-phenoxy}-phenyl)-2-methyl-benzamide,
N-(4-{2-Ethoxy-4-[3-(1-ethyl-propyl)-ureido]-phenoxy}-phenyl)-4-(4-methyl-[1,4]diazepan-1-yl)-benzamide,
N-(4-{2-Ethoxy-4-[3-(1-ethyl-propyl)-ureido]-phenoxy}-phenyl)-4-(4-(ethyl-piperazin-1-yl)-benzamide,
1-(1-Butyl-piperidin-4-yl)-1H-indole-5-carboxylic acid (4-{4-[3-(1-ethyl-propyl)-ureido]-phenoxy}-phenyl)-amide,
4-(4-Butyl-piperazin-1-yl)-N-(4-{2-Ethoxy-4-[3-(1-ethyl-propyl)-ureido]-phenoxy}-phenyl)-benzamide,
4-(4-Butyl-[1,4]diazepan-1-yl)-N-(4-{4-[3-(1-ethyl-propyl)-ureido]-5-fluoro-2-methoxy-phenoxy}-phenyl)-benzamide,
4-(4-Butyl-[1,4]diazepan-1-yl)-N-(4-{4-[3-(1-ethyl-propyl)-ureido]-2-methoxy-phenoxy}-phenyl)-benzamide,
4-(1-Butyl-piperidin-4-yloxy)-N-(4-{4-[3-(1-ethyl-propyl)-ureido]-3-fluoro-phenoxy}phenyl)-3-methyl-benzamide,
4-(1-Butyl-piperidin-4-yloxy)-N-(4-{2-(2-dimethylamino-ethoxy)-4-[3-(1-ethyl-propyl)-ureido]-phenoxy}-2-fluoro-phenyl)-benzamide,
1-(1-Butyl-piperidin-4-yl)-1H-indole-5-carboxylic acid (4-{4-[3-(1-ethyl-propyl)-ureido]-phenoxy}-3-methyl-phenyl)-amide,
4-(4-Butyl-[1,4]diazepan-1-yl)-N-(4-{2-Ethoxy-4-[3-(1-ethyl-propyl)-ureido]-phenoxy}-phenyl)-benzamide,
4-(1-Butyl-piperidin-4-yloxy)-N-(4-{4-[3-(1-ethyl-propyl)-ureido]-5-fluoro-2-methoxy-phenoxy}-phenyl)-2-fluoro-5-methyl-benzamide,
4-(1-Butyl-piperidin-4-yloxy)-N-(4-{2-Ethoxy-4-[3-(1-ethyl-propyl)-ureido]-phenoxy}-phenyl)-2-fluoro-5-methyl-benzamide,
N-(4-{2-Ethoxy-4-[3-(1-ethyl-propyl)-ureido]-phenoxy}-phenyl)-4-(4-ethyl-piperazin-1-ylmethyl)-benzamide,
1-(1-Butyl-piperidin-4-yl)-1H-indole-5-carboxylic acid (4-{4-[3-(1-ethyl-propyl)-ureido]-2,5-difluoro-phenoxy}-phenyl)-amide,
4-(1-Butyl-piperidin-4-yloxy)-N-(4-{4-[3-(1-ethyl-propyl)-ureido]-2,5-difluoro-phenoxy}phenyl)-3-methyl-benzamide,
2-Methyl-1,2,3,4-tetrahydro-benzo[4,5]furo[3,2-c]pyridine-8-carboxylic acid (4-{4-[3-(1-ethyl-propyl)-ureido]-2-methoxy-phenoxy}-phenyl)-amide,
4-(1-Butyl-piperidin-4-yloxy)-N-(4-{4-[3-(1-ethyl-propyl)-ureido]-3-fluoro-phenoxy}-3-methyl-phenyl)-benzamide,
4-(4-Butyl-piperazin-1-yl)-N-(4-{2-ethoxy-4-[3-(1-ethyl-propyl)-ureido]-phenoxy}-phenyl)-2-fluoro-5-methyl-benzamide,
4-(1-Butyl-piperidin-4-yloxy)-N-(4-{4-[3-(1-ethyl-propyl)-ureido]-2-methoxy-phenoxy}-phenyl)-N-tetrahydro-pyran-4-yl)-benzamide,
4-(1-Butyl-piperidin-4-yloxy)-N-(4-{4-[3-(1-ethyl-propyl)-ureido]-2-methoxy-phenoxy}-phenyl)-N-(2-methoxy-1-methyl-ethyl)-benzamide,
4-(1-Butyl-piperidin-4-yloxy)-N-(4-{4-[3-(1-ethyl-propyl)-ureido]-2-methoxy-phenoxy}-phenyl)-N-(2-methoxy-propyl)-benzamide,
4-(1-Butyl-piperidin-4-yloxy)-N-(4-{4-[3-(1-ethyl-propyl)-ureido]-2-methoxy-phenoxy}-phenyl)-N-tetrahydro-furan-3-yl)-benzamide,
4-(1-Butyl-piperidin-4-yloxy)-N-(4-{4-[3-(1-ethyl-propyl)-ureido]-2-methoxy-phenoxy}phenyl)-N-tetrahydro-furan-2-ylmethyl)-benzamide,
4-(1-Butyl-piperidin-4-yloxy)-N-ethyl-N-(4-{4-[3-(1-ethyl-propyl)-ureido]-5-fluoro-2-methoxy-phenoxy}-phenyl-3-methyl-benzamide,
4-(1-Butyl-piperidin-4-yloxy)-N-(4-{4-[3-(1-ethyl-propyl)-ureido]-5-fluoro-2-methoxy-phenoxy}-phenyl)-N-(2-methoxy-ethyl)-3-methyl-benzamide,
4-(1-Butyl-piperidin-4-yloxy)-N-(4-{2-ethoxy-4-[3-(1-ethyl-propyl)-ureido]-5-fluoro-phenoxy}-phenyl)-3-methyl-benzamide,
4-(1-Butyl-piperidin-4-yloxy)-N-{4-[5-fluoro-4-(3-isopropyl-ureido)-2-methoxy-phenoxy]-phenyl}3-methyl-benzamide,
4-(1-Butyl-piperidin-4-yloxy)-N-(4-{2-ethoxy-4-[3-(1-ethyl-propyl)-ureido]-5-fluoro-phenoxy}-phenyl)-2-fluoro-5-methyl-benzamide,
(1-Ethyl-propyl)-carbamate of 4-{4-[4-(1-butyl-piperidin-4-yloxy)-3-methyl-benzoylamino]-phenoxy}-3-methoxy-phenyl,
4-(1-Butyl-piperidin-4-yloxy)-N-(4-{5-fluoro-2-methoxy-4-[3-(1-methoxymethyl-propyl)-ureido]-phenoxy}-phenyl)-3-methyl-benzamide,
4-(1-Butyl-piperidin-4-yloxy)-N-(4-{4-[3-(1-ethyl-propyl)-ureido]-5-fluoro-2-methoxy-phenoxy}-phenyl)-3-methyl-benzamide,
N-{4-[5-Fluoro-4-(3-isopropyl-ureido)-2-methoxy-phenoxy]-phenyl}-4-[1-(3-methoxy-propyl)-piperidin-4-yloxy]-3-methyl-benzamide,
4-(4-Butyl-[1,4]diazepan-1-yl-N-(4-{2-ethoxy-4-[3-(1-ethyl-propyl)-ureido]-phenoxy}-phenyl)-2,5-difluoro-benzamide,
4-(1-Butyl-piperidin-4-yloxy)-N-(4-{4-[3-(1-ethyl-propyl)-ureido]-2-methoxy-phenoxy}-phenyl)-2,5-difluoro-benzamide,
4-[1-(2-Ethoxy-ethyl)-piperidin-4-yloxy]-N-(4-{4-[3-(1-ethyl-propyl)-ureido]-5-fluoro-2-methoxy-phenoxy}-phenyl)-3-methyl-benzamide,
N-(4-{4-[3-(1-Ethyl-propyl)-ureido]-5-fluoro-2-methoxy-phenoxy}-phenyl)-4-[1-(2-methoxy-ethyl)-piperidin-4-yloxy]-3-methyl-benzamide,
N-(4-{4-[3-(1-Ethyl-propyl)-ureido]-5-fluoro-2-methoxy-phenoxy}-phenyl)-4-[1-(3-methoxy-propyl)-piperidin-4-yloxy]-3-methyl-benzamide,
4-(1-Butyl-piperidin-4-ylamino)-N-(4-{2-ethoxy-4-[3-(1-ethyl-propyl)-ureido]-phenoxy}-phenyl)-benzamide,
N-(4-{2-Ethoxy-4-[3-(1-ethyl-propyl)-ureido]-phenoxy}-phenyl)-4-(1-ethyl-piperidin-4-ylamino)-benzamide,
N-(2-Dimethylamino-ethyl)-N-(4-{4-[3-(1-ethyl-propyl)-ureido]-2-methoxy-phenoxy}-phenyl)-4-(1-methyl-piperidin-4-yloxy)-benzamide,
N-(4-{4-[3-(1-Ethyl-propyl)-ureido]-2-methoxy-phenoxy}-phenyl)-4-{1-[3-(tetrahydro-pyran-4-ylamino)-propyl]-piperidin-4-yloxy}-benzamide,
4-[(1-Butyl-piperidin-4-yl)-methyl-amino]-N-(4-{2-ethoxy-4-[3-(1-ethyl-propyl)-ureido]-phenoxy}-phenyl)-benzamide,
4-(1-Butyl-piperidin-4-yloxy)-N-(4-{4-[3-(1-ethyl-propyl)-ureido]-3-fluoro-phenoxy}-3-methyl-phenyl)₃₋₂-hydroxy-ethyl)-benzamide,
N-(4-{4-[3-(1-Ethyl-propyl)-ureido]-2-methoxy-phenoxy}-phenyl)-N-(2-methoxy-ethyl)-4-(piperidin-4-yloxy)-benzamide,
N-(4-{4-[3-(1-Ethyl-propyl)-ureido]-2,5-difluoro-phenoxy}-phenyl)-4-piperidin-4-yloxy)-benzamide,
N-(4-{2-Ethoxy-4-[3-(1-ethyl-propyl)-ureido]-phenoxy}-phenyl)-4-(methyl-piperidin-4-yl-amino)-benzamide,
N-(4-{2-Ethoxy-4-[3-(1-ethyl-propyl)-ureido]-phenoxy}-phenyl)-4-[(1-ethyl-piperidin-4-yl)methyl-amino]-benzamide,
1-(4-{4-[4-(1-Butyl-piperidin-4-yloxy)-benzoyl]-3,4-dihydro-2H-benzo[1,4]oxazin-7-yloxy}phenyl)-3-(1-ethyl-propyl)-urea,
N-(4-{4-[3-(1-Ethyl-propyl)-ureido]-3-fluoro-phenoxy}-2-fluoro-phenyl)-4-(1-methyl-piperidin-4-yloxy)-benzamide,
4-(1-Butyl-piperidin-4-yloxy)-N-(4-{4-[3-(1-ethyl-propyl)-ureido]-3-fluoro-phenoxy}-2-fluoro-phenyl)-3-methyl-benzamide,
1-(1-Butyl-piperidin-4-yl)-1H-indole-5-carboxylic acid (4-{4-[3-(1-ethyl-propyl)-ureido]-3-fluoro-phenoxy}-2-fluoro-phenyl)-amide,
1-(4-{9-[4-(1-Butyl-piperidin-4-yloxy)-benzoyl]-6,7,8,9-tetrahydro-5-oxa-9-aza-benzocyclohepten-3-yloxy}-3-methoxy-phenyl)-3-(1-ethyl-propyl)-urea,
4-(1-Butyl-piperidin-4-yloxy)-piperidine-1-carboxylic acid (4-{2-ethoxy-4-[3-(1-ethyl-propyl)-ureido]-phenoxy}-phenyl)-amide, and
1-(4-{4-[4-(1-Butyl-piperidin-4-yloxy)-phenoxymethyl]-phenoxy}-3-methoxy-phenyl)-3-(1-ethyl-propyl)-urea,
and their pharmaceutically acceptable salts, their solvates and hydrates, their optical and geometric isomers and their mixtures.

42. The compound according to claim 24, which is selected from the group consisting of:
4-(1-Benzyl-piperidin-4-yloxy)-N-(4-{4-[3-(1-ethyl-propyl)-ureido]-5-fluoro-2-methoxy-phenoxy}-phenyl)-3-methyl-benzamide,
N-(4-{4-[3-(1-Ethyl-propyl)-ureido]-5-fluoro-2-methoxy-phenoxy}-phenyl)-3-methyl-4-(piperidin-4-yloxy)-benzamide,
4-(1-Benzyl-piperidin-4-ylamino)-N-(4-{2-ethoxy-4-[3-(1-ethyl-propyl)-ureido]-phenoxy}-phenyl)-benzamide,
N-(4-{2-Ethoxy-4-[3-(1-ethyl-propyl)-ureido]-phenoxy}-phenyl)-4-piperidin-4-ylamino)-benzamide,
N-(2-Dimethylamino-ethyl-N-(4-{4-[3-(1-ethyl-propyl)-ureido]-2-methoxy-phenoxy}-phenyl)-4-(piperidin-4-yloxy)-benzamide,
4-[(1-Benzyl-piperidin-4-yl)-methyl-amino]-N-(4-{2-ethoxy-4-[3-(1-ethyl-propyl)-ureido]-phenoxy}-phenyl)-benzamide,
N-(4-{4-[3-(1-Ethyl-propyl)-ureido]-3-fluoro-phenoxy}-3-methyl-phenyl)-3-(2-hydroxy-ethyl)-4-(piperidin-4-yloxy)-benzamide,
4-(1-Benzyl-Piperidin-4-yloxy)-N-(4-{4-[3-(1-ethyl-propyl)-ureido]-phenoxy}-3-methyl-phenyl)-benzamide,
N-(4-{4-[3-(1-Ethyl-propyl)-ureido]-2-methoxy-phenoxy}-3-methyl-phenyl)-4-(Piperidin-4-yloxy)-benzamide,
4-(1-Benzyl-Piperidin-4-yloxy)-N-(4-{4-[3-(1-ethyl-propyl)-ureido]-2-methoxy-phenoxy}-3-methyl-phenyl)-benzamide,
N-(4-{4-[3-(1-Ethyl-propyl)-ureido]-2-methoxy-phenoxy}-phenyl)-N-isobutyl-4-(Piperidin-4-yloxy)-benzamide,
N-(4-{4-[3-(1-Ethyl-propyl)-ureido]-2-methoxymethyl-phenoxy}-phenyl)-4-(Piperidin yloxy)-benzamide,
4-(1-Benzyl-Piperidin-4-yloxy)-N-(4-{4-[3-(1-ethyl-propyl)-ureido]-2-methoxymethyl-phenoxy}-phenyl)-benzamide,
and the pharmaceutically acceptable salts, solvates and hydrates, optical and geometric isomers and mixtures of these compounds.

43. A pharmaceutical composition comprising at least one compound according to claim 24, in a pharmaceutically acceptable support.

44. A method for the treatment of obesity, abnormal eating behaviour or to control food intake, or to treat excess fat, Type II diabetes or metabolic syndrome, hypertension, a vascular disease, Raynaud's disease, pheochromocytoma, angina, to combat coronary and cerebral vasospasm, to treat atherosclerosis, heart failure or ischaemia, anorexia, depression, anxiety, sexual behaviour disorders, to treat drug or alcohol addiction or dependence problems, to treat pain, inflammation, allergy, or a gastro-intestinal disorder, or to regulate the onset of puberty, comprising administering to a subject in need of such treatment an effective amount of at least one compound of formula (I) as defined in claim 24.

45. A method according to claim 46, wherein the abnormal eating behaviour is bulimia or wherein the gastrointestinal disorder is Irritable Bowel Syndrome (IBS), Inflammatory Bowel Disease (IBD) or Crohn's disease.

46. A method for antagonizing the neuropeptide Y1 receptor comprising treating cells overexpressing neuropeptide Y with an neuropeptide receptor antagonist amount of at least one compound of formula (I) as defined in claim 24.

47. A method for the treatment of a disease in which the activity of neuropeptide Y is abnormally high comprising administering to a subject in need of such treatment an effective neuropeptide Y receptor antagonist amount of at least one compound of formula (I) as defined in claim 24.
